(19) United States
(12) Patent Application Publication
(10) Pub. No.: US 2024/0167059 A1
(43) Pub. Date: May 23, 2024

(54) SYSTEMS AND METHODS FOR TARGETED INTEGRATION AND GENOME EDITING AND DETECTION THEREOF USING INTEGRATED PRIMING SITES

(71) Applicant: Editas Medicine, Inc., Cambridge, MA (US)

(72) Inventors: Cecilia Cotta-Ramusino, Cambridge, MA (US); Carrie M. Margulies, Waban, MA (US)

(21) Appl. No.: 18/520,878

(22) Filed: Nov. 28, 2023

Related U.S. Application Data
(63) Continuation of application No. 16/630,510, filed on Jan. 13, 2020, now Pat. No. 11,866,726.
(60) Provisional application No. 62/582,563, filed on Nov. 7, 2017.
(60) Provisional application No. 62/532,509, filed on Jul. 14, 2017.

(51) Int. Cl. C12N 15/90; C12N 5/0735; C12N 5/074; C12N 5/0789; C12N 9/22; C12N 15/10; C12N 15/113; C12N 15/86

(52) U.S. Cl. CPC C12N 15/902; C12N 5/0606; C12N 5/0647; C12N 5/0696; C12N 9/22; C12N 15/102; C12N 15/113; C12N 15/86; C12N 2310/20

(57) ABSTRACT

Disclosed herein are genome editing systems and related methods which allow for the detection and quantitative measurement of all possible on-target gene editing outcomes, including targeted integration. The compositions and methods described herein rely on the use of donor templates comprising a 5′ homology arm, a cargo, a one or more priming sites, a 3′ homology arm, and optionally stuffer sequence.

### RELATED APPLICATIONS

This application is a continuation application of U.S. patent application Ser. No. 16/630,510 filed on Jan. 13, 2020, which is a U.S. national stage application, filed under 35 U.S.C. § 371, of International Application No. PCT/US2018/042040, filed on Jul. 13, 2018, which claims the benefit of U.S. Provisional Patent Application No. 62/582,563, filed on Nov. 7, 2017, and of U.S. Provisional Patent Application No. 62/532,509, filed on Jul. 14, 2017. The entire contents of each of the foregoing applications are expressly incorporated herein by reference.

### SEQUENCE LISTING

The instant application contains a Sequence Listing which has been submitted electronically in XML format and is hereby incorporated by reference in its entirety. Said XML copy, created on Dec. 1, 2023, is named 126454-01904_SL.xml and is 366,426 bytes in size.

### FIELD

This disclosure relates to genome editing systems and methods and compositions for editing a target nucleic acid sequence, or modulating expression of a target nucleic acid sequence, and applications thereof.

### BACKGROUND

CRISPRs (Clustered Regularly Interspaced Short Palindromic Repeats) evolved in bacteria and archea as an adaptive immune system to defend against viral attack. Upon exposure to a virus, short segments of viral DNA are integrated into the CRISPR locus. RNA is transcribed from a portion of the CRISPR locus that includes the viral sequence. That RNA, which contains sequence complementary to the viral genome, mediates targeting of a Cas9 protein to a target sequence in the viral genome. The Cas9 protein, in turn, cleaves and thereby silences the viral target.

Recently, the CRISPR/Cas system has been adapted for genome editing in eukaryotic cells. The introduction of site-specific double strand breaks (DSBs) allows for target sequence alteration through endogenous DNA repair mechanisms, for example non-homologous end-joining (NHEJ) or homology-directed repair (HDR).

In addition, targeted integration of a nucleic acid (e.g., a transgene) may be achieved using the CRISPR/Cas system. Applications of targeted integration include the delivery of therapeutic transgene expression cargos to cleavage sites in the genome that are amenable to robust expression and tolerant to insertions. Moreover, when endogenous control of gene expression is required, targeted integration of a corrected gene/exon at the site corresponding to the endogenous gene/exon is an attractive strategy.

Targeted integration of the exogenous DNA cassette (e.g., transgene) with high-efficiency and accuracy is the ultimate goal. However, quantitative assessment of targeted integration is not straightforward. Currently, targeted integration is primarily detected and measured using two separate methods: flow cytometric analysis of transgene expression and digital droplet PCR (ddPCR). The limitation of flow cytometry is that the method relies on the detection of a phenotypic output which may not correlate with actual genomic integration. For example, the integrated nucleic acid may be epigenetically silenced which may lead to a lower perceived integration rate. The integrated nucleic acid may also integrate at an off-target location or an un-integrated donor template may be expressed by the cell, which may lead to an increased perceived integration rate. Thus, under these alternative scenarios, the resulting detection of integration frequency may be inaccurate.

ddPCR is an on-target quantitative assay that may be used to measure targeted integration. A donor template is often provided along with a DNA nuclease to induce a double- or single-stranded DNA break from which repair is initiated. Although ddPCR provides an accurate rate of targeted integration, it does not provide information about other types of on-target editing, such as insertions or deletions (indels). Indel measurements can be made via other methods, which often have biases. Multiple methods are sometimes combined to fully characterize on-target editing, but the results are usually not ideal and often inaccurate. Therefore, there remains a need in the art for methods to detect and quantitatively measure all possible on-target gene editing outcomes, including targeted integration.

### SUMMARY

The present disclosure provides genome editing systems and related methods which allow for the detection and quantitative measurement of on-target gene editing outcomes, including targeted integration. The compositions and methods described herein rely on the use of donor templates comprising a 5′ homology arm, a cargo, a one or more priming sites, a 3′ homology arm, and optionally stuffer sequence.

The compositions and methods described herein allow for the quantitative analysis of on-target gene editing outcomes, including targeted integration events, by embedding one or more primer binding sites (i.e., priming sites) into a donor template that are substantially identical to a priming site present at the targeted genomic DNA locus (i.e., the target nucleic acid). The priming sites are embedded into the donor template such that, when homologous recombination of the donor template with a target nucleic acid occurs, successful targeted integration of the donor template integrates the priming sites from the donor template into the target nucleic acid such that at least one amplicon can be generated in order to quantitatively determine the on-target editing outcomes.

In some embodiments, the target nucleic acid comprises a first priming site (P1) and a second priming site (P2), and the donor template comprises a cargo sequence, a first priming site (P1′), and a second priming site (P2′), wherein P2′ is located 5′ from the cargo sequence, wherein P1′ is located 3′ from the cargo sequence (i.e., A1--P2′--N--P1′--A2), wherein P1′ is substantially identical to P1, and wherein P2′ is substantially identical to P2. After accurate homology-driven targeted integration, three amplicons are produced using a single PCR reaction with two oligonucleotide primers (FIG. 1A). The first amplicon, Amplicon X, is generated from the primer binding sites originally present in the genomic DNA (P1 and P2), and may be sequenced to analyze on-target editing events that do not result in targeted integration (e.g., insertions, deletions, gene conversion). The remaining two amplicons are mapped to the 5′ and 3′ junctions after homology-driven targeted integration. The second amplicon, Amplicon Y, results from the amplification of the nucleic acid sequence between P1 and P2′ following a targeted integration event at the target nucleic acid, thereby amplifying the 5′ junction. The third amplicon, Amplicon Z, results from the amplification of the nucleic acid sequence between P1′ and P2 following a targeted integration event at the target nucleic acid, thereby amplifying the 3′ junction. Sequencing of these amplicons provides a quantitative assessment of targeted integration at the target nucleic acid, in addition to information about the fidelity of the targeted integration. To avoid any biases inherent to amplicon size, stuffer sequence may optionally be included in the donor template to keep all three expected amplicons the same length.

In one aspect, disclosed herein is an isolated nucleic acid for homologous recombination with a target nucleic acid having a cleavage site, wherein:
(a) a first strand of the target nucleic acid comprises, from 5′ to 3′, P1--H1--X--H2--P2, wherein
P1 is a first priming site;
H1 is a first homology arm;
X is the cleavage site;
H2 is a second homology arm; and
P2 is a second priming site; and
(b) a first strand of the isolated nucleic acid comprises, from 5′ to 3′, A1--P2′--N--A2, or A1--N--P1′--A2, wherein

A1 is a homology arm that is substantially identical to H1;
P2′ is a priming site that is substantially identical to P2;
N is a cargo;
P1′ is a priming site that is substantially identical to P1; and
A2 is a homology arm that is substantially identical to H2.

In one embodiment, the first strand of the isolated nucleic acid comprises, from 5′ to 3′, A1--P2′--N--P1′--A2. In one embodiment, the first strand of the isolated nucleic acid further comprises S1 or S2, wherein the first strand of the isolated nucleic acid comprises, from 5′ to 3′,
A1--S1--P2′--N--A2, or A1--N--P1′--S2--A2;
wherein S1 is a first stuffer, wherein S2 is a second stuffer, and wherein each of S1 and S2 comprise a random or heterologous sequence having a GC content of approximately 40%.

In one embodiment, the first stuffer has a sequence having less than 50% sequence identity to any nucleic acid sequence within 500 base pairs of the cleavage site, and wherein the second stuffer has a sequence having less than 50% sequence identity to any nucleic acid sequence within 500 base pairs of the cleavage site. In one embodiment, the first stuffer has a sequence comprising at least 10 nucleotides of a sequence set forth in Table 2, and wherein the second stuffer has a sequence comprising at least 10 nucleotides of a sequence set forth in Table 2. In one embodiment, the first stuffer has a sequence that is not the same as the sequence of the second stuffer.

In one embodiment, the first strand of the isolated nucleic acid comprises, from 5′ to 3′, A1--S1--P2′--N--P1′--S2--A2. In one embodiment, A1+S1 and A2+S2 have sequences that are of approximately equal length. In one embodiment, A1+S1 and A2+S2 have sequences that are of equal length. In one embodiment, A1+S1 and H1+X+H2 have sequences that are of approximately equal length. In one embodiment, A1+S1 and H1+X+H2 have sequences that are of equal length. In one embodiment, A2+S2 and H1+X+H2 have sequences that are of approximately equal length. In one embodiment, A2+S2 and H1+X+H2 have sequences that are of equal length.

In one embodiment, A1 has a sequence that is at least 40 nucleotides in length, and A2 has a sequence that is at least 40 nucleotides in length.

In one embodiment, A1 has a sequence that is identical to, or differs by no more than 1, 2, 3, 4, 5, 6, 7, 8, 9, 10, 15, 20, 25, or 30 nucleotides from a sequence of H1. In one embodiment, A2 has a sequence that is identical to, or differs by no more than 1, 2, 3, 4, 5, 6, 7, 8, 9, 10, 15, 20, 25, or 30 nucleotides from a sequence of H2.

In one embodiment, A1+S1 have a sequence that is at least 40 nucleotides in length, and A2+S2 have a sequence that is at least 40 nucleotides in length.

In one embodiment, N comprises an exon of a gene sequence, an intron of a gene sequence, a cDNA sequence, or a transcriptional regulatory element; a reverse complement of any of the foregoing or a portion of any of the foregoing. In one embodiment, N comprises a promoter sequence.

In one aspect, disclosed herein is a composition comprising an isolated nucleic acid disclosed herein and, optionally, a pharmaceutically acceptable carrier.

In one aspect, disclosed herein is a vector comprising an isolated nucleic acid disclosed herein. In one embodiment, the vector is a plasmid vector. In one embodiment, the vector is a viral vector. In one embodiment, the vector is an AAV vector, an adenoviral vector, a lentiviral vector, an integration-deficient lentiviral vector (IDLV), a retroviral vector, a HSV vector, a naked DNA vector, or a lipid nanoparticle.

In one aspect, disclosed herein is a genome editing system comprising an isolated nucleic acid disclosed herein. In one embodiment, the genome editing system further comprises a RNA-guided nuclease and at least one gRNA molecule.

In one aspect, disclosed herein is a method of altering a cell comprising contacting the cell with a genome editing system.

In one aspect, disclosed herein is a kit comprising a genome editing system.

In one aspect, disclosed herein is a nucleic acid, composition, vector, gene editing system, method or kit, for use in medicine.

In one aspect, disclosed herein is a method of altering a cell, comprising the steps of: forming, in a target nucleic acid of the cell, at least one single- or double-strand break at a cleavage site, wherein the target nucleic acid comprises a first strand comprising: a first homology arm 5′ to the cleavage site, a first priming site either within the first homology arm or 5′ to the first homology arm, a second homology arm 3′ to the cleavage site, and a second priming site either within the second homology arm or 3′ to the second homology arm, and recombining an exogenous oligonucleotide donor template with the target nucleic acid by homologous recombination to produce an altered nucleic acid, wherein a first strand of the exogenous oligonucleotide donor template comprises either: i) a cargo, a priming site that is substantially identical to the second priming site either within or 5′ to the cargo, a first donor homology arm 5′ to the cargo, and a second donor homology arm 3′ to the cargo; or ii) a cargo, a first donor homology arm 5′ to the cargo, a priming site that is substantially identical to the first priming site either within or 3′ to the cargo, and a second donor homology arm 3′ to the cargo, thereby altering the cell.

In one embodiment, the first strand of the exogenous oligonucleotide donor template comprises, from 5′ to 3′, the first donor homology arm, the priming site that is substantially identical to the second priming site, the cargo, the priming site that is substantially identical to the first priming site, and the second donor homology arm. In one embodiment, the first strand of the exogenous oligonucleotide donor template further comprises a first stuffer or a second stuffer, wherein the first stuffer and the second stuffer each comprise a random or heterologous sequence having a GC content of approximately 40%; and wherein the first strand of the exogenous oligonucleotide donor template comprises, from 5′ to 3′, i) the first donor homology arm, the first stuffer, the priming site that is substantially identical to the second priming site, and the second donor homology arm; or ii) the first donor homology arm, the cargo, the priming site that is substantially identical to the first priming site, the second stuffer, and the second donor homology arm.

In one embodiment, the first stuffer has a sequence having less than 50% sequence identity to any nucleic acid sequence within 500 base pairs of the cleavage site, and wherein the second stuffer has a sequence having less than 50% sequence identity to any nucleic acid sequence within 500 base pairs of the cleavage site. In one embodiment, the first stuffer has a sequence comprising at least 10 nucleotides of a sequence set forth in Table 2, and wherein the second stuffer has a sequence comprising at least 10 nucleotides of a sequence set forth in Table 2. In one embodiment, the first stuffer has a sequence that is not the same as the sequence of the second stuffer.

In one embodiment, the first strand of the exogenous oligonucleotide donor template comprises, from 5′ to 3′, the first donor homology arm, the first suffer, the priming site that is substantially identical to the second priming site, the cargo, the priming site that is substantially identical to the first priming site, the second stuffer, and the second donor homology arm.

In one embodiment, the altered nucleic acid comprises, from 5′ to 3′, the first priming site, the first donor homology arm, the priming site that is substantially identical to the second priming site, the cargo, the second donor homology arm, and the second priming site. In one embodiment, the altered nucleic acid comprises, from 5′ to 3′, the first priming site, the first donor homology arm, the cargo, the priming site that is substantially identical to the first priming site, the second donor homology arm, and the second priming site.

In one embodiment, the altered nucleic acid comprises, from 5′ to 3′, the first priming site, the first donor homology arm, the priming site that is substantially identical to the second priming site, the cargo, the priming site that is substantially identical to the first priming site, the second donor homology arm, and the second priming site.

In one embodiment, the altered nucleic acid comprises, from 5′ to 3′, the first priming site, the first donor homology arm, the first stuffer, the priming site that is substantially identical to the second priming site, the cargo, the priming site that is substantially identical to the first priming site, the second stuffer, the second donor homology arm, and the second priming site.

In one embodiment, the step of forming the at least one single- or double-strand break comprises contacting the cell with an RNA-guided nuclease. In one embodiment, the RNA-guided nuclease is a Class 2 Clustered Regularly Interspersed Repeat (CRISPR)-associated nuclease. In one embodiment, the RNA-guided nuclease is selected from the group consisting of wild-type Cas9, a Cas9 nickase, a wild-type Cpf1, and a Cpf1 nickase. In another embodiment, the step of forming the at least one single- or double-strand break comprises contacting the cell with a Zinc finger Nuclease (ZFN). In another embodiment, the step of forming the at least one single- or double-strand break comprises contacting the cell with a Transcription activator-like effector nuclease (TALEN).

In one embodiment, the step of contacting the RNA-guided nuclease with the cell comprises introducing into the cell a ribonucleoprotein (RNP) complex comprising the RNA-guided nuclease and a guide RNA (gRNA). In one embodiment, the step of recombining the exogenous oligonucleotide donor template into the nucleic acid by homologous recombination comprises introducing the exogenous oligonucleotide donor template into the cell.

In one embodiment, the step of introducing comprises electroporation of the cell in the presence of the RNP complex and/or the exogenous oligonucleotide donor template.

In another aspect, disclosed herein is a method of integrating an exogenous nucleic acid into a target nucleic acid in a cell, comprising (a) introducing the exogenous nucleic acid into the cell, wherein a first strand of the target nucleic acid comprises, from 5′ to 3′, P1--H1--X--H2--P2, wherein P1 is a first priming site; H1 is a first homology arm; X is a cleavage site; H2 is a second homology arm; and P2 is a second priming site; and wherein a first strand of the isolated nucleic acid comprises, from 5′ to 3′, A1--P2′--N--A2, or A1--N--P1′--A2, wherein A1 is a homology arm that is substantially identical to H1; P2′ is a priming site that is substantially identical to P2; N is a cargo; P1′ is a priming site that is substantially identical to P1; and A2 is a homology arm that is substantially identical to H2; and (b) introducing one or more nucleases into the cell, wherein the nucleases cleave the target nucleic acid at the cleavage site, such that the exogenous nucleic acid is integrated into the target nucleic acid.

In one embodiment, the first strand of the exogenous nucleic acid comprises, from 5′ to 3′, A1--P2′--N--P1′--A2.

In one embodiment, the first strand of the exogenous nucleic acid further comprises S1 or S2, wherein the first strand of the isolated nucleic acid comprises, from 5′ to 3′, A1--S1--P2′--N--A2, or A1--N--P1′--S2--A2; wherein S1 is a first stuffer, wherein S2 is a second stuffer. In one embodiment, the first strand of the isolated nucleic acid comprises, from 5′ to 3′, A1--S1--P2′--N--P1′--S2--A2.

In one embodiment, each of S1 and S2 comprise a random or heterologous sequence having a GC content of approximately 40%. Additional features of the stuffer sequences are described herein. For example, in one embodiment, the first stuffer has a sequence having less than 50% sequence identity to any nucleic acid sequence within 500 base pairs of the cleavage site, and wherein the second stuffer has a sequence having less than 50% sequence identity to any nucleic acid sequence within 500 base pairs of the cleavage site. In one embodiment, the first stuffer has a sequence comprising at least 10 nucleotides of a sequence set forth in Table 2, and wherein the second stuffer has a sequence comprising at least 10 nucleotides of a sequence set forth in Table 2.

In one embodiment, the first stuffer has a sequence that is not the same as the sequence of the second stuffer. In one embodiment, A1+S1 and A2+S2 have sequences that are of approximately equal length. In one embodiment, A1+S1 and A2+S2 have sequences that are of equal length. In one embodiment, A1+S1 and H1+X+H2 have sequences that are of approximately equal length. In one embodiment, A1+S1 and H1+X+H2 have sequences that are of equal length. In one embodiment, A2+S2 and H1+X+H2 have sequences that are of approximately equal length. In one embodiment. A2+S2 and H1+X+H2 have sequences that are of equal length.

The cargo sequence (N) can comprise any nucleic acid sequence to be integrated into the target nucleic acid. For example, in some embodiments, the cargo sequence can comprise an exon of a gene sequence, an intron of a gene sequence, a cDNA sequence, or a transcriptional regulatory element; a reverse complement of any of the foregoing or a portion of any of the foregoing. In one embodiment. N comprises a promoter sequence.

In one embodiment, following integration of the exogenous nucleic acid, the target nucleic acid comprises, from 5′ to 3′, the first priming site, the first donor homology arm, the priming site that is substantially identical to the second priming site, the cargo, the second donor homology arm, and the second priming site. In one embodiment, the altered nucleic acid comprises, from 5′ to 3′, the first priming site, the first donor homology arm, the cargo, the priming site that is substantially identical to the first priming site, the second donor homology arm, and the second priming site.

In another embodiment, following integration of the exogenous nucleic acid, the target nucleic acid comprises, from 5′ to 3′, the first priming site, the first donor homology arm, the priming site that is substantially identical to the second priming site, the cargo, the priming site that is substantially identical to the first priming site, the second donor homology arm, and the second priming site.

In another embodiment, following integration of the exogenous nucleic acid, the target nucleic acid comprises, from 5′ to 3′, the first priming site, the first donor homology arm, the first stuffer, the priming site that is substantially identical to the second priming site, the cargo, the priming site that is substantially identical to the first priming site, the second stuffer, the second donor homology arm, and the second priming site.

In one embodiment, the nuclease is an RNA-guided nuclease. In one embodiment, the RNA-guided nuclease is a Class 2 Clustered Regularly Interspersed Repeat (CRISPR)-associated nuclease. In one embodiment, the RNA-guided nuclease is selected from the group consisting of wild-type Cas9, a Cas9 nickase, a wild-type Cpf1, and a Cpf1 nickase. In one embodiment, the RNA-guided nuclease is provided as a ribonucleoprotein (RNP) complex comprising the RNA-guided nuclease and a guide RNA (gRNA). In another embodiment, the method further comprises introducing one or more gRNAs into the cell. In one embodiment, the nuclease is a Zinc finger Nuclease (ZFN). In another embodiment, the nuclease is a Transcription activator-like effector nuclease (TALEN).

In one embodiment, the exogenous nucleic acid is provided as naked nucleic acid, e.g., naked DNA, or naked RNA. In another embodiment, the exogenous nucleic acid is provided in a vector. The vector can be, for example, a plasmid vector. In other embodiments, the vector can be a viral vector, for example, an AAV vector, an adenoviral vector, a lentiviral vector, an IDLV vector, a retroviral vector, or a herpes simplex virus (HSV) vector. In other embodiments, the vector is a naked DNA vector. In other embodiments, the exogenous nucleic acid is provided in a lipid nanoparticle.

In one aspect, disclosed herein is a method of altering a target nucleic acid in a cell, wherein the target nucleic acid comprises a first strand comprising: a first homology arm 5′ to a cleavage site, a first priming site either within the first homology arm or 5′ to the first homology arm, a second homology arm 3′ to the cleavage site, and a second priming site either within the second homology arm or 3′ to the second homology arm, the method comprising: contacting the cell with (a) at least one gRNA molecule, (b) a RNA-guided nuclease molecule, and (c) an exogenous oligonucleotide donor template, wherein a first strand of the exogenous oligonucleotide donor template comprises either: i) a cargo, a priming site that is substantially identical to the second priming site either within or 5′ to the cargo, a first donor homology arm 5′ to the cargo, and a second donor homology arm 3′ to the cargo; or ii) a cargo, a first donor homology arm 5′ to the cargo, a priming site that is substantially identical to the first priming site, and a second donor homology arm 3′ to the cargo; wherein the gRNA molecule and the RNA-guided nuclease molecule interact with the target nucleic acid, resulting in a cleavage event at or near the cleavage site, and wherein the cleavage event is repaired by at least one DNA repair pathway to produce an altered nucleic acid, thereby altering the target nucleic acid in the cell.

In one embodiment, the method further comprises contacting the cell with (d) a second gRNA molecule, wherein the second gRNA molecule and the RNA-guided nuclease molecule interact with the target nucleic acid, resulting in a second cleavage event at or near the cleavage site, and wherein the second cleavage event is repaired by the at least one DNA repair pathway.

In one embodiment, the first strand of the exogenous oligonucleotide donor template comprises, from 5′ to 3′, the first donor homology arm, the priming site that is substantially identical to the second priming site, the cargo, the priming site that is substantially identical to the first priming site, and the second donor homology arm.

In one embodiment, the first strand of the exogenous oligonucleotide donor template further comprises a first stuffer or a second stuffer, wherein the first stuffer and the second stuffer each comprise a random or heterologous sequence having a GC content of approximately 40%; and wherein the first strand of the exogenous oligonucleotide donor template comprises, from 5′ to 3′, i) the first donor homology arm, the first stuffer, the priming site that is substantially identical to the second priming site, and the second donor homology arm; or ii) the first donor homology arm, the cargo, the priming site that is substantially identical to the first priming site, the second stuffer, and the second donor homology arm.

In one embodiment, the first stuffer has a sequence having less than 50% sequence identity to any nucleic acid sequence within 500 base pairs of the cleavage site, and wherein the second stuffer has a sequence having less than 50% sequence identity to any nucleic acid sequence within 500 base pairs of the cleavage site. In one embodiment, the first stuffer has a sequence comprising at least 10 nucleotides of a sequence set forth in Table 2, and wherein the second stuffer has a sequence comprising at least 10 nucleotides of a sequence set forth in Table 2. In one embodiment, the first stuffer has a sequence that is not the same as the sequence of the second stuffer.

In one embodiment, the first strand of the exogenous oligonucleotide donor template comprises, from 5′ to 3′, the first donor homology arm, the first suffer, the priming site that is substantially identical to the second priming site, the cargo, the priming site that is substantially identical to the first priming site, the second stuffer, and the second donor homology arm.

In one embodiment, the altered nucleic acid comprises, from 5′ to 3′, the first priming site, the first donor homology arm, the priming site that is substantially identical to the second priming site, the cargo, the second donor homology arm, and the second priming site. In one embodiment, the altered nucleic acid comprises, from 5′ to 3′, the first priming site, the first donor homology arm, the cargo, the priming site that is substantially identical to the first priming site, the second donor homology arm, and the second priming site.

In one embodiment, the altered nucleic acid comprises, from 5′ to 3′, the first priming site, the first donor homology arm, the priming site that is substantially identical to the second priming site, the cargo, the priming site that is substantially identical to the first priming site, the second donor homology arm, and the second priming site.

In one embodiment, the altered nucleic acid comprises, from 5′ to 3′, the first priming site, the first donor homology arm, the first stuffer, the priming site that is substantially identical to the second priming site, the cargo, the priming site that is substantially identical to the first priming site, the second stuffer, the second donor homology arm, and the second priming site.

In one embodiment, the cell is contacted first with the at least one gRNA molecule and the RNA-guided nuclease molecule, followed by contacting the cell with the exogenous oligonucleotide donor template. In one embodiment, the cell is contacted with the at least one gRNA molecule, the RNA-guided nuclease molecule, and the exogenous oligonucleotide donor template at the same time.

In one embodiment, the exogenous oligonucleotide donor template is present in a vector. In one embodiment, the vector is a viral vector. In another embodiment, the vector is a plasmid vector. In another embodiment, the vector is naked DNA. In one embodiment, the viral vector is an AAV vector, an adenoviral vector, a lentiviral vector, or an integration-deficient lentiviral vector (IDLV).

In one embodiment, the DNA repair pathway repairs the target nucleic acid to result in targeted integration of the exogenous oligonucleotide donor template. In one embodiment, the altered nucleic acid comprises a sequence comprising an indel as compared to a sequence of the target nucleic acid. In one embodiment, the cleavage event, or both the cleavage event and the second cleavage event, is/are repaired by gene correction.

In one embodiment, the first donor homology arm and the first stuffer consist of a sequence that is of approximately equal length to a sequence consisting of the second donor homology arm and the second stuffer. In one embodiment, the first donor homology arm and the first stuffer consist of a sequence that is of equal length to the sequence consisting of the second donor homology arm and the second stuffer.

In one embodiment, the first donor homology arm and the first stuffer consist of a sequence that is of approximately equal length to a sequence consisting of the first homology arm, the cleavage site, and the second homology arm. In one embodiment, the first donor homology arm and the first stuffer consist of a sequence that is of equal length to a sequence consisting of the first homology arm, the cleavage site, and the second homology arm.

In one embodiment, the second donor homology arm and the second stuffer consist of a sequence that is of approximately equal length to a sequence consisting of the first homology arm, the cleavage site, and the second homology arm. In one embodiment, the second donor homology arm and the second stuffer consist of a sequence that is of equal length to a sequence consisting of the first homology arm, the cleavage site, and the second homology arm.

In one embodiment, the first donor homology arm has a sequence that is at least 40 nucleotides in length, and wherein the second donor homology arm has a sequence that is at least 40 nucleotides in length. In one embodiment, the first donor homology arm has a sequence that is identical to, or differs by no more than 1, 2, 3, 4, 5, 6, 7, 8, 9, 10, 15, 20, 25, or 30 nucleotides from, a sequence of the first homology arm. In one embodiment, the second donor homology arm has a sequence that is identical to, or differs by no more than 1, 2, 3, 4, 5, 6, 7, 8, 9, 10, 15, 20, 25, or 30 nucleotides from, a sequence of the second homology arm.

In one embodiment, the first donor homology arm and the first stuffer consist of a sequence that is at least 40 nucleotides in length, and the second donor homology arm and the second stuffer consist of a sequence that is at least 40 nucleotides in length.

In one embodiment, the first suffer has a sequence that is different from a sequence of the second stuffer.

In one embodiment, the first priming site, the priming site that is substantially identical to the first priming site, the second priming site, and the priming site that is substantially identical to the second priming site are each less than 60 base pairs in length.

In one embodiment, the method further comprises amplifying the target nucleic acid, or a portion of the target nucleic acid, prior to the forming step or the contacting step.

In one embodiment, the method further comprises amplifying the altered nucleic acid using a first primer which binds to the first priming site and/or the priming site that is substantially identical to the first priming site, and a second primer which binds to the second priming site and/or the priming site that is substantially identical to the second priming site.

In one embodiment, the altered nucleic acid comprises a sequence that is different than a sequence of the target nucleic acid.

In one embodiment, the gRNA molecule is a gRNA nucleic acid, and wherein the RNA-guided nuclease molecule is a RNA-guided nuclease protein. In one embodiment, the gRNA molecule is a gRNA nucleic acid, and wherein the RNA-guided nuclease molecule is a RNA-guided nuclease nucleic acid. In one embodiment, the cell is contacted with the gRNA molecule and the RNA-guided nuclease molecule as a pre-formed complex. In one embodiment, the RNA-guided nuclease is selected from the group consisting of wild-type Cas9, a Cas9 nickase, a wild-type Cpf1, and a Cpf1 nickase.

In one embodiment, the target nucleic acid comprises an exon of a gene, an intron of a gene, a cDNA sequence, a transcriptional regulatory element; a reverse complement of any of the foregoing; or a portion of any of the foregoing.

In one embodiment, the cell is a eukaryotic cell. In one embodiment, the eukaryotic cell is a human cell. In one embodiment, the cell is a peripheral blood cell. In one embodiment, the cell is a CD34+ cell. In one embodiment, the eukaryotic cell is an immune cell, an epithelial cell, an endothelial cell, a neuron, or a stem cell. In one embodiment, the eukaryotic cell is an immune cell. In an exemplary embodiment, the immune cell is a T cell, a B cell, a natural killer cell, a monocyte, a macrophage cell, or a dendritic cell. In one embodiment, the eukaryotic is a T cell, e.g., a helper T cell, a regulatory T cell (Treg), or a Natural Killer T cell (NK T cell). In another embodiment, the eukaryotic cell is a stem cell. In exemplary embodiments, the stem cell is an embryonic stem cell, an induced pluripotent stem cell (iPSC), or a hematopoietic stem/progenitor cell (HSPC).

In one embodiment, the cell is from a subject suffering from a disease or disorder. In one embodiment, the disease or disorder is a blood disease, an immune disease, a neurological disease, a cancer, an infectious disease, a genetic disease, a disorder caused by aberrant mtDNA, a metabolic disease, a disorder caused by aberrant cell cycle, a disorder caused by aberrant angiogenesis, a disorder cause by aberrant DNA damage repair, or a pain disorder.

In one embodiment, the cell is from a subject having at least one mutation at the cleavage site.

In one embodiment, the method further comprises isolating the cell from the subject prior to contacting the forming step or the contacting step.

In one embodiment, the method further comprises introducing the cell into a subject after the recombining step or after the cleavage event is repaired by the at least one DNA repair pathway.

In one embodiment, the forming step and the recombining step, or the contacting step, is performed in vitro. In one embodiment, the forming step and the recombining step, or the contacting step, is performed ex vivo. In one embodiment, the forming step and the recombining step, or the contacting step, is performed in vivo.

In another embodiment, the cell is a plant cell. In one embodiment, the plant is monocotyledonous, for example, maize, wheat, rice, corn, or Setaria. In another embodiment, the plant is dicotyledonous, for example, potato, soybean, tomato, tobacco, or Arabidopsis. In one aspect, disclosed herein is a method for determining the outcome of a gene editing event at a cleavage site in a target nucleic acid in a cell using an exogenous donor template, wherein the target nucleic acid comprises a first strand comprising: a first homology arm 5′ to a cleavage site, a first priming site either within the first homology arm or 5′ to the first homology arm, a second homology arm 3′ to the cleavage site, and a second priming site either within the second homology arm or 3′ to the second homology arm, and wherein a first strand of the exogenous donor template comprises i) a cargo, a priming site that is substantially identical to the second priming site either within or 5′ to the cargo, a first donor homology arm 5′ to the cargo, and a second donor homology arm 3′ to the cargo; or ii) a cargo, a first donor homology arm 5′ to the cargo, a priming site that is substantially identical to the first priming site 3′ to the cargo, and a second donor homology arm 3′ to the cargo, the method comprising: i) forming at least one single- or double-strand break at or near the cleavage site in the target nucleic acid; ii) recombining the exogenous oligonucleotide donor template with the target nucleic acid via homologous recombination to produce an altered nucleic acid; and iii) amplifying the altered nucleic acid using a first primer which binds to the first priming site and/or the priming site that is substantially identical to the first priming site; and/or a second primer which binds to the second priming site and/or the priming site that is substantially identical to the second priming site; thereby determining the outcome of the gene editing event in the cell.

In one embodiment, the step of forming the at least one single- or double-strand break comprises contacting the cell with an RNA-guided nuclease. In one embodiment, the RNA-guided nuclease is a Class 2 Clustered Regularly Interspersed Repeat (CRISPR)-associated nuclease. In one embodiment, the RNA-guided nuclease is selected from the group consisting of wild-type Cas9, a Cas9 nickase, a wild-type Cpf1, and a Cpf1 nickase. In another embodiment, the step of forming the at least one single- or double-strand break comprises contacting the cell with a Zinc finger Nuclease (ZFN). In another embodiment, the step of forming the at least one single- or double-strand break comprises contacting the cell with a Transcription activator-like effector nuclease (TALEN).

In one embodiment, the step of contacting the RNA-guided nuclease with the cell comprises introducing into the cell a ribonucleoprotein (RNP) complex comprising the RNA-guided nuclease and at least one guide RNA (gRNA). In one embodiment, the step of recombining the exogenous oligonucleotide donor template into the nucleic acid via homologous recombination comprises introducing the exogenous oligonucleotide donor template into the cell. In one embodiment, the step of introducing comprises electroporation of the cell in the presence of the RNP complex and/or the exogenous oligonucleotide donor template.

In one embodiment, the first strand of the exogenous oligonucleotide donor template comprises, from 5′ to 3′, the first donor homology arm, the priming site that is substantially identical to the second priming site, the cargo, the priming site that is substantially identical to the first priming site, and the second donor homology arm.

In one embodiment, the first strand of the exogenous oligonucleotide donor template further comprises a first stuffer and/or a second stuffer, wherein the first stuffer and the second stuffer each comprise a random or heterologous sequence having a GC content of approximately 40%; and wherein the exogenous oligonucleotide donor template comprises, from 5′ to 3′, i) the first donor homology arm, the first stuffer, the priming site that is substantially identical to the second priming site, and the second donor homology arm; or ii) the first donor homology arm, the cargo, the priming site that is substantially identical to the first priming site, the second stuffer, and the second donor homology arm.

In one embodiment, the first stuffer has a sequence having less than 50% sequence identity to any nucleic acid sequence within 500 base pairs of the cleavage site, and wherein the second stuffer has a sequence having less than 50% sequence identity to any nucleic acid sequence within 500 base pairs of the cleavage site. In one embodiment, the first stuffer has a sequence comprising at least 10 nucleotides of a sequence set forth in Table 2, and wherein the second stuffer has a sequence comprising at least 10 nucleotides of a sequence set forth in Table 2. In one embodiment, the first stuffer has a sequence that is not the same as the sequence of the second stuffer.

In one embodiment, the first strand of the exogenous oligonucleotide donor template comprises, from 5′ to 3′, the first donor homology arm, the first suffer, the priming site that is substantially identical to the second priming site, the cargo, the priming site that is substantially identical to the first priming site, the second stuffer, and the second donor homology arm.

In one embodiment, the altered nucleic acid comprises, from 5′ to 3′, the first priming site, the first donor homology arm, the priming site that is substantially identical to the second priming site, the cargo, the second donor homology arm, and the second priming site. In one embodiment, the altered nucleic acid comprises, from 5′ to 3′, the first priming site, the first donor homology arm, the cargo, the priming site that is substantially identical to the first priming site, the second donor homology arm, and the second priming site.

In one embodiment, the altered nucleic acid comprises, from 5′ to 3′, the first priming site, the first donor homology arm, the priming site that is substantially identical to the second priming site, the cargo, the priming site that is substantially identical to the first priming site, the second donor homology arm, and the second priming site.

In one embodiment, the altered nucleic acid comprises, from 5′ to 3′, the first priming site, the first donor homology arm, the first stuffer, the priming site that is substantially identical to the second priming site, the cargo, the priming site that is substantially identical to the first priming site, the second stuffer, the second donor homology arm, and the second priming site.

In one embodiment, when the altered nucleic acid comprises a non-targeted integration genome editing event at the cleavage site, amplifying the altered nucleic acid using the first primer and the second primer produces a first amplicon, wherein the first amplicon has a sequence that comprises an indel as compared to a sequence of the target nucleic acid.

In one embodiment, when the altered nucleic acid comprises a targeted integration genome editing event at the cleavage site, amplifying the altered nucleic acid using the first primer and the second primer produces a first amplicon, wherein the first amplicon has a sequence that is substantially identical to a sequence consisting of either i) the first donor homology arm and the first stuffer, or ii) the second stuffer and the second donor homology arm.

In one embodiment, when the altered nucleic acid comprises a targeted integration genome editing event at the cleavage site, amplifying the altered nucleic acid using the first primer and the second primer produces a first amplicon and a second amplicon, wherein the first amplicon has a sequence that is substantially identical to a sequence consisting of the first donor homology arm and the first stuffer, and wherein the second amplicon has a sequence that is substantially identical to a sequence consisting of the second stuffer and the second homology arm.

In one embodiment, the cell is a population of cells, and when the altered nucleic acid in all cells within the population of cells comprises a non-targeted integration genome editing event at the cleavage site, amplifying the altered nucleic acid using the first primer and the second primer produces a first amplicon, wherein the first amplicon has a sequence that comprises an indel as compared to a sequence of the target nucleic acid.

In one embodiment, the cell is a population of cells, and when the altered nucleic acid in all the cells within the population of cells comprises a targeted integration genome editing event at the cleavage site, amplifying the altered nucleic acid using the first primer and the second primer produces a first amplicon, wherein the first amplicon has a sequence that is substantially identical to a sequence consisting of either i) the first donor homology arm and the first stuffer, or ii) the second stuffer and the second donor homology arm.

In one embodiment, the cell is a population of cells, and when the altered nucleic acid in a first cell within the population of cells comprises a non-targeted integration genome editing event at the cleavage site, amplifying the altered nucleic acid using the first primer and the second primer produces a first amplicon, wherein the first amplicon has a sequence that comprises an indel as compared to a sequence of the target nucleic acid; and when the altered nucleic acid in a second cell within the population of cells comprises a targeted integration genome editing event at the cleavage site, amplifying the altered nucleic acid in the second cell using the first primer and the second primer produces a second amplicon, wherein the second amplicon has a sequence that is substantially identical to a sequence consisting of either i) the first donor homology arm and the first stuffer, or ii) the second stuffer and the second donor homology arm.

In one embodiment, the cell is a population of cells, when the altered nucleic acid in a first cell within the population of cells comprises a non-targeted integration genome editing event at the cleavage site, amplifying the altered nucleic acid using the first primer and the second primer produces a first amplicon, wherein the first amplicon has a sequence that comprises an indel as compared to a sequence of the target nucleic acid; and when the altered nucleic acid in a second cell within the population of cells comprises a targeted integration genome editing event at the cleavage site, amplifying the altered nucleic acid in the second cell using the first primer and the second primer produces a second amplicon and a third amplicon, wherein the second amplicon has a sequence that is substantially identical to a sequence consisting of the first donor homology arm and the first stuffer, and wherein the third amplicon has a sequence that is substantially identical to a sequence consisting of the second stuffer and the second donor homology arm.

In one embodiment, frequency of targeted integration versus non-targeted integration in the population of cells can be measured by: i) the ratio of ((an average of the second amplicon plus the third amplicon)/(first amplicon plus (the average of the second amplicon plus the third amplicon)); ii) the ratio of (the second amplicon/(the first amplicon plus the second amplicon)); or iii) the ratio of (the third amplicon/(the first amplicon plus the third amplicon)).

In one aspect, disclosed herein is a cell, or a population of cells, altered by a method disclosed herein.

In another aspect, disclosed herein is a cell, or a population of cells, comprising a donor template disclosed herein.

In one aspect, the invention provides a cell comprising an altered nucleic acid, wherein a first strand of the altered nucleic acid comprises, from 5′ to 3′, a first priming site, a first donor homology arm, a cargo, a priming site that is substantially identical to the first priming site, a second donor homology arm, and a second priming site. In another aspect, the invention provides a cell comprising an altered nucleic acid, wherein a first strand of the altered nucleic acid comprises, from 5′ to 3′, a first priming site, a first donor homology arm, a priming site that is substantially identical to a second priming site, a cargo, a second donor homology arm, and the second priming site. In another aspect, the invention provides a cell comprising an altered nucleic acid, wherein a first strand of the altered nucleic acid comprises, from 5′ to 3′, a first priming site, a first donor homology arm, a priming site that is substantially identical to a second priming site, a cargo, a priming site that is substantially identical to the first priming site, a second donor homology arm, and the second priming site.

In one embodiment, the altered nucleic acid further comprises a first stuffer and/or a second stuffer. For example, the altered nucleic acid can comprise, from 5′ to 3′, (i) a first priming site, a first donor homology arm, a cargo, a priming site that is substantially identical to the first priming site, a first stuffer, a second donor homology arm, and a second priming site; (ii) a first priming site, a first donor homology arm, a first stuffer, a priming site that is substantially identical to a second priming site, a cargo, a second donor homology arm, and the second priming site; or (iii) a first priming site, a first donor homology arm, a first stuffer, a priming site that is substantially identical to a second priming site, a cargo, a priming site that is substantially identical to the first priming site, a second stuffer, a second donor homology arm, and the second priming site.

In an exemplary embodiment, the cell is derived from a cell comprising an unaltered nucleic acid, wherein a first strand of the unaltered nucleic acid comprises, from 5′ to 3′, the first priming site, a first homology arm substantially identical to the first donor homology arm, a cleavage site, a second homology arm substantially identical to the second donor homology arm, and the second priming site.

In one embodiment, the cell is a eukaryotic cell. In one embodiment, the eukaryotic cell is a human cell. In one embodiment, the cell is a peripheral blood cell. In one embodiment, the cell is a CD34+ cell. In one embodiment, the eukaryotic cell is an immune cell, an epithelial cell, an endothelial cell, a neuron, or a stem cell. In one embodiment, the eukaryotic cell is an immune cell. In an exemplary embodiment, the immune cell is a T cell, a B cell, a natural killer cell, a monocyte, a macrophage cell, or a dendritic cell. In another embodiment, the eukaryotic cell is a stem cell. In exemplary embodiments, the stem cell is an embryonic stem cell, an induced pluripotent stem cell (iPSC), or a hematopoietic stem/progenitor cell (HSPC).

In one embodiment, the cell is from a subject suffering from a disease or disorder. In one embodiment, the disease or disorder is a blood disease, an immune disease, a neurological disease, a cancer, an infectious disease, a genetic disease, a disorder caused by aberrant mtDNA, a metabolic disease, a disorder caused by aberrant cell cycle, a disorder caused by aberrant angiogenesis, a disorder cause by aberrant DNA damage repair, or a pain disorder.

In another embodiment, the cell is a plant cell. In one embodiment, the plant is monocotyledonous, for example, maize, wheat, rice, corn, or Setaria. In another embodiment, the plant is dicotyledonous, for example, potato, soybean, tomato, tobacco, or Arabidopsis.

### BRIEF DESCRIPTION OF THE DRAWINGS

The accompanying drawings are intended to provide illustrative, and schematic rather than comprehensive, examples of certain aspects and embodiments of the present disclosure. The drawings are not intended to be limiting or binding to any particular theory or model, and are not necessarily to scale. Without limiting the foregoing, nucleic acids and polypeptides may be depicted as linear sequences, or as schematic two- or three-dimensional structures; these depictions are intended to be illustrative rather than limiting or binding to any particular model or theory regarding their structure.

FIG. 1A is a schematic representation of an unedited genomic DNA targeting site, an exemplary DNA donor template for targeted integration, potential insertion outcomes (i.e., non-targeted integration at the cleavage site or targeted integration at the cleavage site) and three potential PCR amplicons resulting from use of a primer pair targeting the P1 priming site and the P2 primer site (Amplicon X), a primer pair targeting the PI primer site and the P2′ priming site (Amplicon Y), or a primer pair targeting the P1′ primer site and the P2 primer site (Amplicon Z). The depicted exemplary DNA donor template contains integrated primer sites (P1′ and P2′) and stuffer sequences (S1 and S2). A1/A2: donor homology arms, S1/S2: donor stuffer sequences, P1/P2: genomic primer sites, P1′/P2′: integrated primer sites, H1/H2: genomic homology arms, N: cargo, X: cleavage site.

FIG. 1B is a schematic representation of an unedited genomic DNA targeting site, an exemplary DNA donor template for targeted integration, potential insertion outcomes (i.e., non-targeted integration at the cleavage site or targeted integration at the cleavage site), and two potential PCR amplicons resulting from the use of a primer pair targeting the PI primer site and the P2 primer site (Amplicon X), or a primer pair targeting the P1′ primer site and the P2 primer site (Amplicon Y). The exemplary DNA donor template contains an integrated primer site (P1′) and a stuffer sequence (S2). A1/A2: donor homology arms, S1/S2: donor stuffer sequences, P1/P2: genomic primer sites, P1′: integrated primer sites, H1/H2: genomic homology arms, N: cargo, X: cleavage site.

FIG. 1C is a schematic representation of an unedited genomic DNA targeting site, an exemplary DNA donor template for targeted integration, potential insertion outcomes (i.e., non-targeted integration at the cleavage site or targeted integration at the cleavage site), and two potential PCR amplicons resulting from the use of a primer pair targeting the PI primer site and the P2 primer site (Amplicon X), or a primer pair targeting the P1 primer site and the P2′ primer site (Amplicon Y). The exemplary DNA donor template contains an integrated primer site (P2′) and a stuffer sequence (S1). A1/A2: donor homology arms, S1/S2: donor stuffer sequences, P1/P2: genomic primer sites, P2′: integrated primer sites, H1/H2: genomic homology arms, N: cargo, X: cleavage site.

FIG. 2A depicts exemplary DNA donor templates comprising either long homology arms (“500 bp HA”), short homology arms (“177 bp HA”), or no homology arms (“No HA”) used for targeted integration experiments in primary CD4+ T-cells using wild-type S. pyogenes ribonucleoprotein targeted to the HBB locus. FIG. 2B, FIG. 2C, and FIG. 2D depict that DNA donor templates with either long homology arms and short homology arms have similar targeted integration efficiency in CD4+ T-cells as measured using GFP expression and ddPCR (5′ and 3′ junctions). FIG. 2B shows the GFP fluorescence of CD4+ T-cells contacted with wild-type S. pyogenes ribonucleoprotein and one of the DNA donor templates depicted in FIG. 2A at different multiplicities of infection (MOI). FIG. 2C shows the integration frequency in CD4+ T cells contacted with wild-type S. pyogenes ribonucleoprotein (RNP) and one of the DNA donor templates depicted in FIG. 2A at different multiplicities of infection (MOI), as determined using ddPCR amplifying the 5′ integration junction. FIG. 2D shows the integration frequency in CD4+ T cells contacted with wild-type S. pyogenes ribonucleoprotein (RNP) and one of the DNA donor templates depicted in FIG. 2A at different multiplicities of infection (MOI), as determined using ddPCR amplifying the 3′ integration junction

FIG. 3 depicts the quantitative assessment of on-target editing events from sequencing at HBB locus as determined using Sanger sequencing.

FIG. 4 depicts the experimental schematic for evaluation of HDR and targeted integration in CD34+ cells.

FIG. 5A depicts the on-target integration as detected by ddPCR analysis of the 5′ vector-genomic DNA junctions on day 7 in gDNA from CD34⁺ cells that were untreated (-) or treated with RNP+AAV6+/− homology arms (HA). FIG. 5B depicts the on-target integration as detected by ddPCR analysis of the 3′ vector-genomic DNA junctions on day 7 in gDNA from CD34⁺ cells that were untreated (-) or treated with RNP+AAV6+/− homology arms (HA). FIG. 5C depicts the percentage of GFP⁺ cells detected on day 7 in the live CD34⁺ cell fraction which shows that the integrated transgene is expressed from a genomic context.

FIG. 6 depicts the DNA sequencing results for the cells treated with RNP+AAV6+/−HA with % gene modification comprised of HDR (targeted integration events and gene conversion) and NHEJ (Insertions, Deletions, Insertions from AAV6 donor).

FIG. 7 depicts the kinetics of CD34+ cell viability up to 7 days after treatment with electroporation alone (EP control), or electroporation with RNP or RNP+AAV6. Viability was measured by AOPI.

FIG. 8 depicts flow cytometry results which show GFP expression in erythroid and myeloid progeny of edited cells. The boxed gate calls out the events that were positive for erythroid (CD235) or myeloid (CD33) surface antigen (quadrant gates). GFP+ events were scored within the myeloid and erythroid cell populations (boxed gates).

FIG. 9A, FIG. 9B, and FIG. 9C provide a comparison of various methods for targeted integration assessment. FIG. 9A depicts the design of an exemplary donor template for testing targeted integration efficiency at the TRAC locus. The donor contains a 5′ homology arm, a stuffer sequence, a first priming site, a P2A self-cleaving peptide, a GFP sequence, a polyA sequence, a second priming site, a stuffer sequence, and a 3′ homology arm. FIG. 9B depicts the targeted integration efficiency of donor templates configured as shown in FIG. 9A at three cut sites in the TRAC locus (TRAC1, TRAC4, and TRAC13). Targeted integration was assessed by flow cytometry measuring GFP expression, ddPCR, and sequencing of amplification products generated from the integrated priming sites. FIG. 9C provides a complete picture of all on-target editing events at the TRAC1, TRAC4, and TRAC13 cut sites. Sequencing of amplification products generated as depicted in FIG. 1A allows for the accurate quantification of insertions, deletions, and targeted integration events.

FIG. 10A, and FIG. 10B provide a comparison of various methods for targeted integration assessment, using donor templates that lack a stuffer sequence. FIG. 10A depicts the design of exemplary donor templates for testing targeted integration efficiency at the TRAC locus, with and without the use of stuffer sequences. The top donor template depicted in FIG. 10A contains a 5′ homology arm, a stuffer sequence, a first priming site, a P2A self-cleaving peptide, a GFP sequence, a polyA sequence, a second priming site, a stuffer sequence, and a 3′ homology arm. The bottom donor template depicted in FIG. 10A contains a 5′ homology arm, a first priming site, a P2A self-cleaving peptide, a GFP sequence, a polyA sequence, a second priming site, and a 3′ homology arm. FIG. 10B depicts the targeted integration efficiency of donor templates configured as in FIG. 10A at three cut sites in the TRAC locus (TRAC1, TRAC4, and TRAC13). Targeted integration was assessed by flow cytometry measuring GFP expression, UDITAS™, and sequencing of amplification products generated from the integrated priming sites.

FIG. 11 depicts the editing outcome and targeted integration frequency in CD4+ T cells at the TRAC4, TRAC1, and TRAC13 locus, following exposure to AAV donors targeting each cut site.

FIG. 12 depicts the editing outcome and targeted integration frequency in CD4+ T cells at the TRAC4 locus, following exposure to AAV donors containing short or long homology arms.

FIG. 13 depicts representative flow cytometry plots of targeted integration and knock out with donors targeting the TRAC4 cut site. GFP expression (indicative of targeted integration) is shown in the Y axis, expression of T cell receptor (TCR) is shown on the X axis. Q1, +GFP/−TCR; Q2, +GFP/+TCR; Q3, −GFP/+TCR; Q4, −GFP/−TCR.

FIG. 14 graphically depicts the UDITAS™ method for detecting and measuring gene editing events.

### DETAILED DESCRIPTION

### Definitions and Abbreviations

Unless otherwise specified, each of the following terms has the meaning associated with it in this section.

The indefinite articles “a” and “an” refer to at least one of the associated noun, and are used interchangeably with the terms “at least one” and “one or more.” For example, “a module” means at least one module, or one or more modules.

The conjunctions “or” and “and/or” are used interchangeably as non-exclusive disjunctions.

“Alt-HDR,” “alternative homology-directed repair,” or “alternative HDR” are used interchangeably to refer to the process of repairing DNA damage using a homologous nucleic acid (e.g., an endogenous homologous sequence, e.g., a sister chromatid, or an exogenous nucleic acid, e.g., a template nucleic acid). Alt-HDR is distinct from canonical HDR in that the process utilizes different pathways from canonical HDR, and can be inhibited by the canonical HDR mediators, RAD51 and BRCA2. Alt-HDR is also distinguished by the involvement of a single-stranded or nicked homologous nucleic acid template, whereas canonical HDR generally involves a double-stranded homologous template.

Unless indicated otherwise, the term “HDR” as used herein encompasses both canonical HDR and alt-HDR.

As used herein, the terms “approximately” or “about” in reference to a number are generally taken to include numbers that fall within a range of 5%, 10%, 15%, or 20% in either direction (greater than or less than) of the number unless otherwise stated or otherwise evident from the context (except where such number would be less than 0% or exceed 100% of a possible value).

The term “approximately equal” is used herein to describe parameters that are identical, nearly identical, and/or functionally identical. The term may be used, for example, in reference to the comparative lengths of portions of a donor template, e.g., the length of A1+S1 of a donor template are “approximately equal” to A2+S2 of the same donor template, such that upon integration of a portion of the donor template into a target nucleic acid, and subsequent amplification using PCR primers, amplicons of similar size having similar amplification efficiencies may be produced, thereby facilitating the quantitation of editing outcomes based on the relative quantities of these amplicons using a single PCR reaction.

The term “approximately equal,” as used herein in reference to the length of two nucleic acid sequences, may also refer to nucleic acid sequences that differ in length, e.g., by less that 25 nucleotides or base pairs. For example, a first sequence is approximately equal in length to a second sequence when the first and second sequences differ in length by less than 25, less than 24, less than 23, less than 22, less than 21, less than 20, less than 19, less than 18, less than 17, less than 16, less than 15, less than 14, less than 13, less than 12, less than 11, less than 10, less than 9, less than 8, less than 7, less than 6, less than 5, less than 4, less than 3, less than 2, or less than 1 nucleotides or base pairs in length. The term “equal” as used herein in reference to the length of two nucleic acid sequences refers to nucleic acid sequences that are the same number of nucleotides or base pairs in length. For example, a first sequence is equal in length to a second sequence when they both consist of, e.g., 25 nucleotides or 25 base pairs.

“Canonical HDR,” “canonical homology-directed repair” or “cHDR” refer to the process of repairing DNA damage using a homologous nucleic acid (e.g., an endogenous homologous sequence, e.g., a sister chromatid, or an exogenous nucleic acid, e.g., a template nucleic acid). Canonical HDR typically acts when there has been significant resection at the double strand break, forming at least one single-stranded portion of DNA. In a normal cell, cHDR typically involves a series of steps such as recognition of the break, stabilization of the break, resection, stabilization of single-stranded DNA, formation of a DNA crossover intermediate, resolution of the crossover intermediate, and ligation. The process requires RAD51 and BRCA2, and the homologous nucleic acid is typically double-stranded.

As used herein, the term “cleavage event” refers to a break in a nucleic acid molecule. A cleavage event may be a single-strand cleavage event, or a double-strand cleavage event. A single-strand cleavage event may result in a 5′ overhang or a 3′ overhang. A double-stranded cleavage event may result in blunt ends, two 5′ overhangs, or two 3′ overhangs.

The term “cleavage site,” as used herein in reference to a site on a target nucleic acid sequence, refers to a target position between two nucleotide residues of the target nucleic acid where a double-stranded break occurs, or alternatively, to a target position within a span of several nucleotide residues of the target nucleic acid wherein two single stranded breaks occur, as mediated by a RNA-guided nuclease-dependent process. A cleavage site may be the target position for, e.g., a blunt double stranded break. Alternatively, a cleavage site may be a target site within a span of several nucleotide residues of the target nucleic acid for, e.g., two single strand breaks or nicks which form a double strand break and which are separated by, e.g., about 10 base pairs. The double strand break(s) or the closer of the two single strand nicks in a pair will ideally be within 0-500 bp of a target position (e.g., no more than 450, 400, 350, 300, 250, 200, 150, 100, 50, or 25 bp from the target position). When dual nickases are used, the two nicks in a pair are within 25-55 bp of each other (e.g., between 25 to 50, 25 to 45, 25 to 40, 25 to 35, 25 to 30, 50 to 55, 45 to 55, 40 to 55, 35 to 55, 30 to 55, 30 to 50, 35 to 50, 40 to 50, 45 to 50, 35 to 45, or 40 to 45 bp) and no more than 100 bp away from each other (e.g., no more than 90, 80, 70, 60, 50, 40, 30, 20, or 10 bp).

“Domain” is used to describe a segment of a protein or nucleic acid. Unless otherwise indicated, a domain is not required to have any specific functional property.

As used herein, the term “endogenous” gene, “endogenous” nucleic acid, or “endogenous” homologous region refers to a native gene, nucleic acid, or region of a gene, which is in its natural location in the genome, e.g., chromosome or plasmid, of a cell. In contrast, the term “exogenous” gene or “exogenous” nucleic acid refers to a gene, nucleic acid, or region of a gene which is not native within a cell, but which is introduced into the cell during the methods of the invention. An exogenous gene or exogenous nucleic acid may be homologous to, or identical to, an endogenous gene or an endogenous nucleic acid.

“Gene conversion” refers to the alteration of a DNA sequence by incorporation of an endogenous homologous sequence (e.g., a homologous sequence within a gene array). “Gene correction” refers to the alteration of a DNA sequence by incorporation of an exogenous homologous sequence, such as an exogenous single- or double-stranded donor template DNA. Gene conversion and gene correction are products of the repair of DNA double-strand breaks by HDR pathways such as those described below.

The terms “homology” or “identity,” as used interchangeably herein, refer to sequence identity between two amino acid sequences or two nucleic acid sequences, with identity being a more strict comparison. The phrases “percent identity or homology” and “% identity or homology” refer to the percentage of sequence identity found in a comparison of two or more amino acid sequences or nucleic acid sequences. Two or more sequences can be anywhere from 0-100% identical, or any value there between. Identity can be determined by comparing a position in each sequence that can be aligned for purposes of comparison to a reference sequence. When a position in the compared sequence is occupied by the same nucleotide base or amino acid, then the molecules are identical at that position. A degree of identity of amino acid sequences is a function of the number of identical amino acids at positions shared by the amino acid sequences. A degree of identity between nucleic acid sequences is a function of the number of identical or matching nucleotides at positions shared by the nucleic acid sequences. A degree of homology of amino acid sequences is a function of the number of amino acids at positions shared by the polypeptide sequences.

Calculations of homology or sequence identity between two sequences (the terms are used interchangeably herein) are performed as follows. The sequences are aligned for optimal comparison purposes (e.g., gaps can be introduced in one or both of a first and a second amino acid or nucleic acid sequence for optimal alignment and non-homologous sequences can be disregarded for comparison purposes). The optimal alignment is determined as the best score using the GAP program in the GCG software package with a Blossum 62 scoring matrix with a gap penalty of 12, a gap extend penalty of 4, and a frame shift gap penalty of 5. The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position. The percent identity between the two sequences is a function of the number of identical positions shared by the sequences.

An “indel” is an insertion and/or deletion in a nucleic acid sequence. An indel may be the product of the repair of a DNA double strand break, such as a double strand break formed by a genome editing system of the present disclosure. An indel is most commonly formed when a break is repaired by an “error prone” repair pathway such as the NHEJ pathway described below.

Indels, gene conversion, gene correction, and other genome editing outcomes are typically assessed by sequencing (most commonly by “next-gen” or “sequencing-by-synthesis” methods, though Sanger sequencing may still be used) and are quantified by the relative frequency of numerical changes (e.g., ±1, ±2 or more bases) at a site of interest among all sequencing reads. DNA samples for sequencing may be prepared by a variety of methods known in the art, and may involve the amplification of sites of interest by polymerase chain reaction (PCR), the capture of DNA ends generated by double strand breaks, as in the GUIDEseq process described in Tsai et al. (Nat. Biotechnol. 34(5): 483 (2016), incorporated by reference herein) or by other means well known in the art. Genome editing outcomes may also be assessed by in situ hybridization methods such as the FiberComb™ system commercialized by Genomic Vision (Bagneux, France), and by any other suitable methods known in the art.

The term “isolated gene” or “isolated nucleic acid” is a gene or nucleic acid that by virtue of its origin or source of derivation is not associated with naturally associated components that accompany it in its native state; is substantially free of other proteins from the same species; is expressed by a cell from a different species; or does not occur in nature. Thus, a nucleic acid that is chemically synthesized or synthesized in a cellular system different from the cell from which it naturally originates will be “isolated” from its naturally associated components. A nucleic acid may also be rendered substantially free of naturally associated components by isolation, using protein purification techniques well known in the art.

Conventional IUPAC notation is used in nucleotide sequences presented herein, as shown in Table 1, below (see also Cornish-Bowden A, Nucleic Acids Res. 1985 May 10; 13(9):3021-30, incorporated by reference herein). It should be noted, however, that “T” denotes “Thymine or Uracil” in those instances where a sequence may be encoded by either DNA or RNA, for example in gRNA targeting domains.

TABLE 1
IUPAC nucleic acid notation

| Character | Base |
|---|---|
| A | Adenine |
| T | Thymine or Uracil |
| G | Guanine |
| C | Cytosine |
| U | Uracil |
| K | G or T/U |
| M | A or C |
| R | A or G |
| Y | C or T/U |
| S | C or G |
| W | A or T/U |
| B | C, G or T/U |
| V | A, C or G |
| H | A, C or T/U |
| D | A, G or T/U |
| N | A, C, G or T/U |

A “kit” refers to any collection of two or more components that together constitute a functional unit that can be employed for a specific purpose. By way of illustration (and not limitation), one kit according to this disclosure can include a guide RNA complexed or able to complex with an RNA-guided nuclease, and accompanied by (e.g., suspended in, or suspendable in) a pharmaceutically acceptable carrier. The kit can be used to introduce the complex into, for example, a cell or a subject, for the purpose of causing a desired genomic alteration in such cell or subject. The components of a kit can be packaged together, or they may be separately packaged. Kits according to this disclosure also optionally include directions for use (DFU) that describe the use of the kit e.g., according to a method of this disclosure. The DFU can be physically packaged with the kit, or it can be made available to a user of the kit, for instance by electronic means.

“Non-homologous end joining” or “NHEJ” refers to ligation mediated repair and/or non-template mediated repair including canonical NHEJ (cNHEJ) and alternative NHEJ (altNHEJ), which in turn includes microhomology-mediated end joining (MMEJ), single-strand annealing (SSA), and synthesis-dependent microhomology-mediated end joining (SD-MMEJ).

The terms “polynucleotide”, “nucleotide sequence”, “nucleic acid”, “nucleic acid molecule”, “nucleic acid sequence”, and “oligonucleotide” refer to a series of nucleotide bases (also called “nucleotides”) in DNA and RNA, and mean any chain of two or more nucleotides. These terms refer to compositions that can be chimeric mixtures or derivatives or modified versions thereof, single-stranded or double-stranded. These terms also refer to compositions that can be modified at the base moiety, sugar moiety, or phosphate backbone, for example, to improve stability of the molecule, its hybridization parameters, etc. A nucleotide sequence typically carries genetic information, including, but not limited to, the information used by cellular machinery to make proteins and enzymes. These terms include double- or single-stranded genomic DNA, RNA, any synthetic and genetically manipulated polynucleotide, and both sense and antisense polynucleotides. These terms also include nucleic acids containing modified bases.

“Prevent,” “preventing,” and “prevention” refer to the prevention of a disease in a mammal, e.g., in a human, including (a) avoiding or precluding the disease; (b) affecting the predisposition toward the disease; or (c) preventing or delaying the onset of at least one symptom of the disease.

The terms “protein,” “peptide” and “polypeptide” are used interchangeably to refer to a sequential chain of amino acids linked together via peptide bonds. The terms include individual proteins, groups or complexes of proteins that associate together, as well as fragments or portions, variants, derivatives and analogs of such proteins. Peptide sequences are presented herein using conventional notation, beginning with the amino or N-terminus on the left, and proceeding to the carboxyl or C-terminus on the right. Standard one-letter or three-letter abbreviations can be used.

“Replacement” or “replaced,” when used with reference to a modification of a molecule (e.g., a nucleic acid or protein), does not require a process limitation but merely indicates that the replacement entity is present.

“Subject” means a human or non-human animal. A human subject can be any age (e.g., an infant, child, young adult, or adult), and may suffer from a disease, or may be in need of alteration of a gene. Alternatively, the subject may be an animal, which term includes, but is not limited to, mammals, birds, fish, reptiles, amphibians, and more particularly non-human primates, rodents (such as mice, rats, hamsters, etc.), rabbits, guinea pigs, dogs, cats, and so on. In certain embodiments of this disclosure, the subject is livestock, e.g., a cow, a horse, a sheep, or a goat. In certain embodiments, the subject is poultry.

As used herein, the term “substantially” refers to the qualitative condition of exhibiting total or near-total extent or degree of a characteristic or property of interest. One of ordinary skill in the biological arts will understand that biological and chemical phenomena rarely, if ever, go to completion and/or proceed to completeness or achieve or avoid an absolute result. The term “substantially” is therefore used herein to capture the potential lack of completeness inherent in many biological and chemical phenomena.

The term “substantially identical” as used herein in reference to two nucleic acid sequences refers, in some embodiments, to a sequence identity of at least 95% between across an at least 20 nucleotide contiguous stretch of each of the two nucleic acid sequences. For example, a first nucleic acid sequence is substantially identical to a second nucleic acid sequence when the first nucleic acid sequence has at least 95%, at least 96%, at least 97%, at least 98%, at least 99%, or 100% identity across an at least 20 nucleotide contiguous stretch of the second nucleic acid sequence. In some embodiments, a first nucleic acid sequence is substantially identical to a second nucleic acid sequence when the first nucleic acid sequence has at least 95% identity across the entire length of the second nucleic acid sequence. In some embodiments, the term “substantially identical” is used in the context of priming sites, and refers to the ability of the priming sites to support priming with the same PCR primer during an amplification reaction. In some embodiments, the term “substantially identical” is used to describe the relationship between the homology arm of a donor template and the homology arm of a target nucleic acid, and refers to a nucleic acid identity between the homology arms that allows for the efficient recombination of the donor template at the target nucleic acid with tolerance for some degree of polymorphism, e.g., to eliminate PAM or protospacer sequences in the recombined locus.

“Target position” as used herein, refers to a site on a target nucleic acid (e.g., the chromosome) that is modified by a RNA-guided nuclease-dependent process. In an embodiment, a target position can be a site between two nucleotides, e.g., adjacent nucleotides, on the target nucleic acid. The target position may comprise one or more nucleotides that are altered, e.g., corrected, by a template nucleic acid. In an embodiment, the target position is within a “target sequence” (e.g., the sequence to which the gRNA binds). In an embodiment, a target position is upstream or downstream of a target sequence (e.g., the sequence to which the gRNA binds).

“Treat,” “treating,” and “treatment” mean the treatment of a disease in a subject (e.g., a human subject), including one or more of inhibiting the disease, i.e., arresting or preventing its development or progression; relieving the disease, i.e., causing regression of the disease state; relieving one or more symptoms of the disease; and curing the disease.

### Overview

The present disclosure concerns genome editing systems comprising a donor template specifically designed to allow for the quantitative assessment of gene editing events that occur upon resolution of a cleavage event at a cleavage site of a target nucleic acid in a cell. The donor template of the genome editing systems described herein is a DNA oligodeoxynucleotides (ODNs), which can be single-stranded (ssODNs) or double-stranded (dsODNs), and can be used to facilitate HDR-based repair of a double-stranded break. The donor template is particularly useful for introducing alterations into a target DNA sequence, inserting a new sequence into the target sequence, or replacing the target sequence altogether. The disclosure provides donor templates comprising a cargo, one or two homology arms and one or more priming sites. The priming site(s) of the donor templates are spatially arranged in such a manner such that the frequency of integration of a portion of the donor template into the target nucleic acid may be readily assessed and quantified.

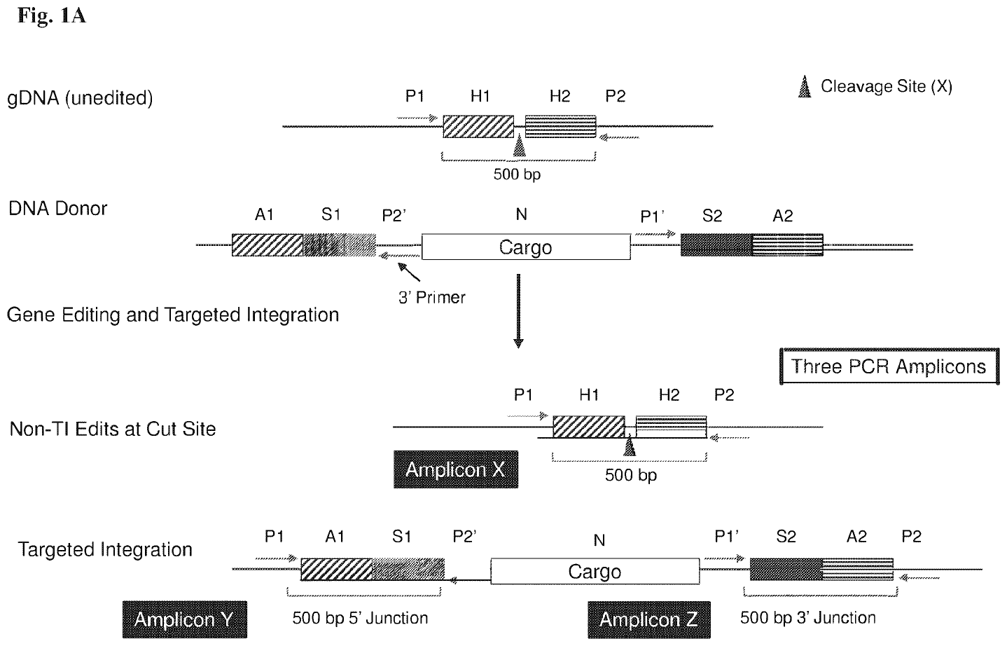

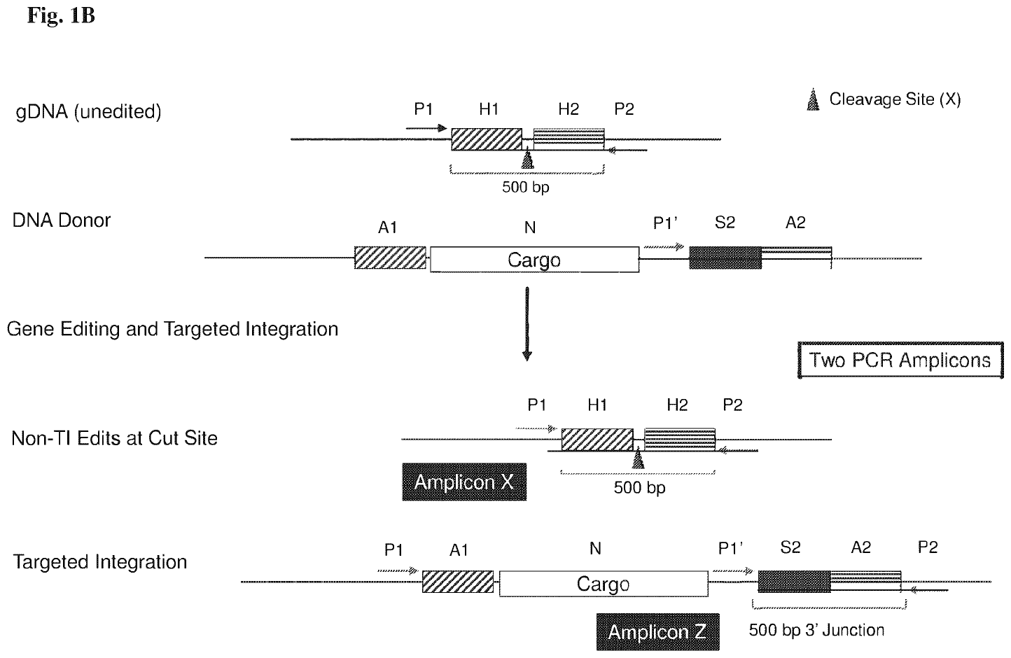

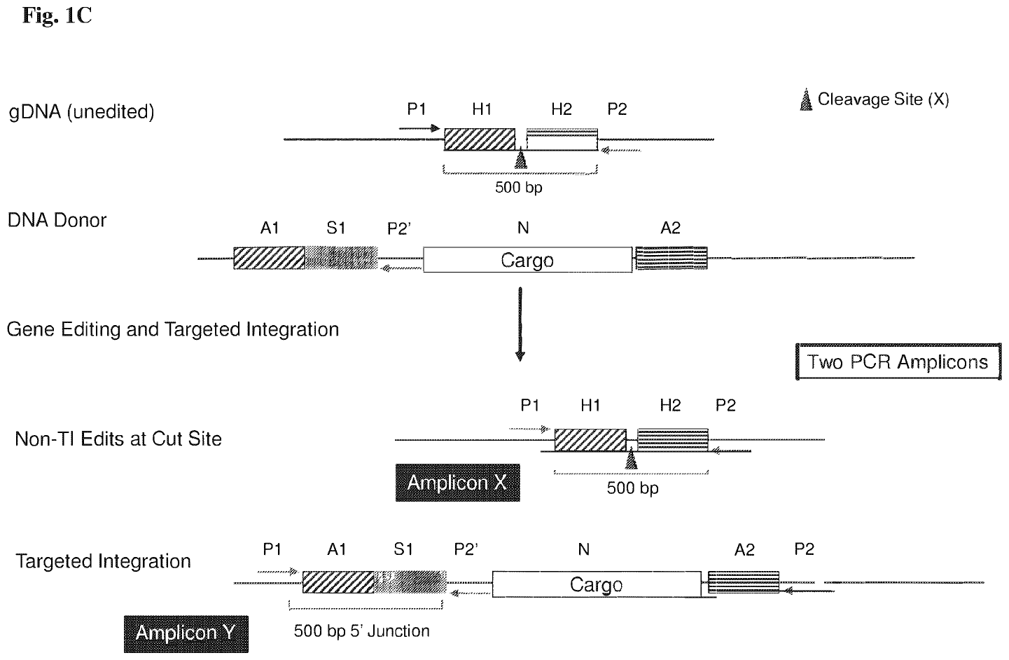

FIGS. 1A, 1B and 1C are diagrams illustrating representative donor templates and the potential targeted integration outcomes resulting from the use of these donor templates. The use of the exemplary donor templates described herein results in the targeted integration of at least one priming site in the targeted nucleic acid which may be used to generate an amplicon that can be sequenced to determine the frequency of targeted integration of a cargo (e.g., a transgene) to the targeted nucleic acid in the target cell.

For example, FIG. 1A illustrates an exemplary donor template comprising from 5′ to 3′, a first homology arm (A1), a first stuffer sequence (S1), a second priming site (P2′), a cargo, a first priming site, a second stuffer sequence, and a second homology arm. The first homology arm (A1) of the donor template is substantially identical to the first homology arm of the target nucleic acid, while the second homology arm (A2) of the donor template is substantially identical to the second homology arm of the target nucleic acid. The donor template is designed such that the second priming site (P2′) is substantially identical to the first priming site of the target nucleic acid (P1), and such that the first priming site (P1′) is substantially identical to the second priming site of the target nucleic acid (P2). Upon resolution of a target nucleic acid cleavage event using a nuclease described herein, a single primer pair set can be used to amplify the nucleic acid sequence surrounding the cleavage site of the target nucleic acid (i.e., the nucleic acid present between P1 and P2, between P1 and P2′, and between P1′ and P2). Advantageously, the size of the amplicons (illustrated as Amplicon X, Y and Z) resulting from resolution of a cleavage event without targeted integration or with targeted integration is approximately the same. The amplicons may then be assessed—for instance by sequencing, or hybridization to a probe sequence—to determine the frequency of targeted integration.

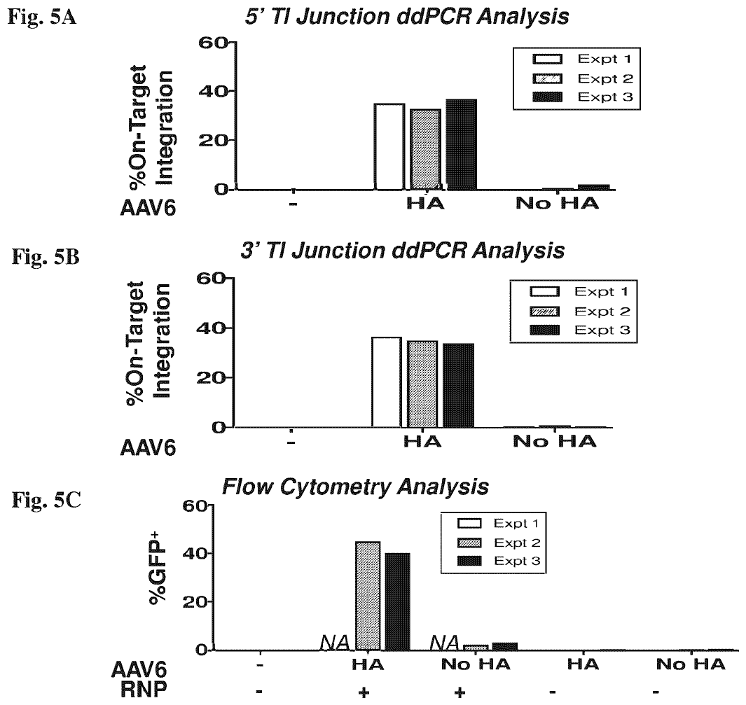

Alternatively, FIGS. 1B and 1C illustrate exemplary donor templates comprising a single priming site that is located either 3′ (FIG. 1B) or 5′ (FIG. 1C) from the cargo nucleic acid sequence. Again, upon resolution of a target nucleic acid cleavage event using a nuclease described herein, these exemplary donor templates are designed such that a single primer pair set can be used to amplify the nucleic acid sequence surrounding the cleavage site of the target nucleic acid, such that two amplicons of approximately the same size are obtained. When the priming site of the donor template is located 3′ from the cargo nucleic acid, amplicons corresponding to a non-targeted integration event, or an amplicon corresponding to the 5′ junction of the targeted integration site may be amplified. When the priming site of the donor template is located 5′ from the cargo nucleic acid, amplicons corresponding to a non-targeted integration event, or an amplicon corresponding to the 3′ junction of the targeted integration site may be amplified. These amplicons may be sequenced to determine the frequency of targeted integration.

### Donor Template

Donor templates according to this disclosure may be implemented in any suitable way, including without limitation single stranded or double stranded DNA, linear or circular, naked or comprised within a vector, and/or associated, covalently or non-covalently (e.g., by direct hybridization or splint hybridization) with a guide RNA. In some embodiments, the donor template is a ssODN. Where a linear ssODN is used, it can be configured to (i) anneal to a nicked strand of the target nucleic acid, (ii) anneal to the intact strand of the target nucleic acid, (iii) anneal to the plus strand of the target nucleic acid, and/or (iv) anneal to the minus strand of the target nucleic acid. An ssODN may have any suitable length, e.g., about, or no more than 150-200 nucleotides (e.g., 150, 160, 170, 180, 190, or 200 nucleotides). In other embodiments, the donor template is a dsODN. In one embodiment, the donor template comprises a first strand. In another embodiment, a donor template comprises a first strand and a second strand. In some embodiments, a donor template is an exogenous oligonucleotide, e.g., an oligonucleotide that is not naturally present in a cell.

It should be noted that a donor template can also be comprised within a nucleic acid vector, such as a viral genome or circular double-stranded DNA, e.g., a plasmid. In some embodiments, the donor template can be a doggy-bone shaped DNA (see, e.g., U.S. Pat. No. 9,499,847). Nucleic acid vectors comprising donor templates can include other coding or non-coding elements. For example, a donor template nucleic acid can be delivered as part of a viral genome (e.g., in an AAV or lentiviral genome) that includes certain genomic backbone elements (e.g., inverted terminal repeats, in the case of an AAV genome) and optionally includes additional sequences coding for a gRNA and/or an RNA-guided nuclease. In certain embodiments, the donor template can be adjacent to, or flanked by, target sites recognized by one or more gRNAs, to facilitate the formation of free DSBs on one or both ends of the donor template that can participate in repair of corresponding SSBs or DSBs formed in cellular DNA using the same gRNAs. Exemplary nucleic acid vectors suitable for use as donor templates are described in Cotta-Ramusino.

A. Homology Arms

Whether single-stranded or double-stranded, donor templates generally include one or more regions that are homologous to regions of DNA, e.g., a target nucleic acid, within or near (e.g., flanking or adjoining) a target sequence to be cleaved, e.g., the cleavage site. These homologous regions are referred to here as “homology arms,” and are illustrated schematically below:
[5′ homology arm]-[replacement sequence]---[3′ homology arm].

The homology arms of the donor templates described herein may be of any suitable length, provided such length is sufficient to allow efficient resolution of a cleavage site on a targeted nucleic acid by a DNA repair process requiring a donor template. In some embodiments, where amplification by, e.g., PCR, of the homology arm is desired, the homology arm is of a length such that the amplification may be performed. In some embodiments, where sequencing of the homology arm is desired, the homology arm is of a length such that the sequencing may be performed. In some embodiments, where quantitative assessment of amplicons is desired, the homology arms are of such a length such that a similar number of amplifications of each amplicon is achieved, e.g., by having similar G/C content, amplification temperatures, etc. In some embodiments, the homology arm is double-stranded. In some embodiments, the double stranded homology arm is single stranded.

In some embodiments, the 5′ homology arm is between 50 to 250 nucleotides in length. In some embodiments, the 5′ homology arm is between 50-2000 nucleotides in length. In some embodiments, the 5′ homology arm is between 50-1500 nucleotides in length. In some embodiments, the 5′ homology arm is between 50-1000 nucleotides in length. In some embodiments, the 5′ homology arm is between 50-500 nucleotides in length. In some embodiments, the 5′ homology arm is between 150 to 250 nucleotides in length. In some embodiments, the 5′ homology arm is 2000 nucleotides or less in length. In some embodiments, the 5′ homology arm is 1500 nucleotides or less in length. In some embodiments, the 5′ homology arm is 1000 nucleotides or less in length. In some embodiments, the 5′ homology arm is 700 nucleotides or less in length. In some embodiments, the 5′ homology arm is 650 nucleotides or less in length. In some embodiments, the 5′ homology arm is 600 nucleotides or less in length. In some embodiments, the 5′ homology arm is 550 nucleotides or less in length. In some embodiments, the 5′ homology arm is 500 nucleotides or less in length. In some embodiments, the 5′ homology arm is 400 nucleotides or less in length. In some embodiments, the 5′ homology arm is 300 nucleotides or less in length. In some embodiments, the 5′ homology arm is 250 nucleotides or less in length. In some embodiments, the 5′ homology arm is 200 nucleotides or less in length. In some embodiments, the 5′ homology arm is 150 nucleotides or less in length. In some embodiments, the 5′ homology arm is less than 100 nucleotides in length. In some embodiments, the 5′ homology arm is 50 nucleotides in length or less. In some embodiments, the 5′ homology arm is 250, 200, 190, 180, 170, 160, 150, 140, 130, 120, 110, 100, 90, 80, 70, 60, 50, 49, 48, 47, 46, 45, 44, 43, 42, 41, 40, 39, 38, 37, 36, 35, 34, 33, 32, 31, 30, 29, 28, 27, 26, 25, 24, 23, 22, 21, or 20 nucleotides in length. In some embodiments, the 5′ homology arm is at least 20 nucleotides in length. In some embodiments, the 5′ homology arm is at least 40 nucleotides in length. In some embodiments, the 5′ homology arm is at least 50 nucleotides in length. In some embodiments, the 5′ homology arm is at least 70 nucleotides in length. In some embodiments, the 5′ homology arm is at least 100 nucleotides in length. In some embodiments, the 5′ homology arm is at least 200 nucleotides in length. In some embodiments, the 5′ homology arm is at least 300 nucleotides in length. In some embodiments, the 5′ homology arm is at least 400 nucleotides in length. In some embodiments, the 5′ homology arm is at least 500 nucleotides in length. In some embodiments, the 5′ homology arm is at least 600 nucleotides in length. In some embodiments, the 5′ homology arm is at least 700 nucleotides in length. In some embodiments, the 5′ homology arm is at least 1000 nucleotides in length. In some embodiments, the 5′ homology arm is at least 1500 nucleotides in length. In some embodiments, the 5′ homology arm is at least 2000 nucleotides in length. In some embodiments, the 5′ homology arm is about 20 nucleotides in length. In some embodiments, the 5′ homology arm is about 40 nucleotides in length. In some embodiments, the 5′ homology arm is 250 nucleotides in length or less. In some embodiments, the 5′ homology arm is about 100 nucleotides in length. In some embodiments, the 5′ homology arm is about 200 nucleotides in length.

In some embodiments, the 3′ homology arm is between 50 to 250 nucleotides in length. In some embodiments, the 3′ homology arm is between 50-2000 nucleotides in length. In some embodiments, the 3′ homology arm is between 50-1500 nucleotides in length. In some embodiments, the 3′ homology arm is between 50-1000 nucleotides in length. In some embodiments, the 3′ homology arm is between 50-500 nucleotides in length. In some embodiments, the 3′ homology arm is between 150 to 250 nucleotides in length. In some embodiments, the 3′ homology arm is 2000 nucleotides or less in length. In some embodiments, the 3′ homology arm is 1500 nucleotides or less in length. In some embodiments, the 3′ homology arm is 1000 nucleotides or less in length. In some embodiments, the 3′ homology arm is 700 nucleotides or less in length. In some embodiments, the 3′ homology arm is 650 nucleotides or less in length. In some embodiments, the 3′ homology arm is 600 nucleotides or less in length. In some embodiments, the 3′ homology arm is 550 nucleotides or less in length. In some embodiments, the 3′ homology arm is 500 nucleotides or less in length. In some embodiments, the 3′ homology arm is 400 nucleotides or less in length. In some embodiments, the 3′ homology arm is 300 nucleotides or less in length. In some embodiments, the 3′ homology arm is 200 nucleotides in length or less. In some embodiments, the 3′ homology arm is 150 nucleotides in length or less. In some embodiments, the 3′ homology arm is 100 nucleotides in length or less. In some embodiments, the 3′ homology arm is 50 nucleotides in length or less. In some embodiments, the 3′ homology arm is 250, 200, 190, 180, 170, 160, 150, 140, 130, 120, 110, 100, 90, 80, 70, 60, 50, 49, 48, 47, 46, 45, 44, 43, 42, 41, 40, 39, 38, 37, 36, 35, 34, 33, 32, 31, 30, 29, 28, 27, 26, 25, 24, 23, 22, 21, or 20 nucleotides in length. In some embodiments, the 3′ homology arm is at least 20 nucleotides in length. In some embodiments, the 3′ homology arm is at least 40 nucleotides in length. In some embodiments, the 3′ homology arm is at least 50 nucleotides in length. In some embodiments, the 3′ homology arm is at least 70 nucleotides in length. In some embodiments, the 3′ homology arm is at least 100 nucleotides in length. In some embodiments, the 3′ homology arm is at least 200 nucleotides in length. In some embodiments, the 3′ homology arm is at least 300 nucleotides in length. In some embodiments, the 3′ homology arm is at least 400 nucleotides in length. In some embodiments, the 3′ homology arm is at least 500 nucleotides in length. In some embodiments, the 3′ homology arm is at least 600 nucleotides in length. In some embodiments, the 3′ homology arm is at least 700 nucleotides in length. In some embodiments, the 3′ homology arm is at least 1000 nucleotides in length. In some embodiments, the 3′ homology arm is at least 1500 nucleotides in length. In some embodiments, the 3′ homology arm is at least 2000 nucleotides in length. In some embodiments, the 3′ homology arm is about 20 nucleotides in length. In some embodiments, the 3′ homology arm is about 40 nucleotides in length. In some embodiments, the 3′ homology arm is 250 nucleotides in length or less. In some embodiments, the 3′ homology arm is about 100 nucleotides in length. In some embodiments, the 3′ homology arm is about 200 nucleotides in length.

In some embodiments, the 5′ homology arm is between 50 to 250 basepairs in length. In some embodiments, the 5′ homology arm is between 50-2000 basepairs in length. In some embodiments, the 5′ homology arm is between 50-1500 basepairs in length. In some embodiments, the 5′ homology arm is between 50-1000 basepairs in length. In some embodiments, the 5′ homology arm is between 50-500 basepairs in length. In some embodiments, the 5′ homology arm is between 150 basepairs to 250 basepairs in length. In some embodiments, the 5′ homology arm is 2000 basepairs or less in length. In some embodiments, the 5′ homology arm is 1500 basepairs or less in length. In some embodiments, the 5′ homology arm is 1000 basepairs or less in length. In some embodiments, the 5′ homology arm is 700 basepairs or less in length. In some embodiments, the 5′ homology arm is 650 basepairs or less in length. In some embodiments, the 5′ homology arm is 600 basepairs or less in length. In some embodiments, the 5′ homology arm is 550 basepairs or less in length. In some embodiments, the 5′ homology arm is 500 basepairs or less in length. In some embodiments, the 5′ homology arm is 400 basepairs or less in length. In some embodiments, the 5′ homology arm is 300 basepairs or less in length. In some embodiments, the 5′ homology arm is 250 basepairs or less in length. In some embodiments, the 5′ homology arm is 200 basepairs or less in length. In some embodiments, the 5′ homology arm is 150 basepairs or less in length. In some embodiments, the 5′ homology arm is less than 100 basepairs in length. In some embodiments, the 5′ homology arm is 50 basepairs in length or less. In some embodiments, the 5′ homology arm is 250, 200, 190, 180, 170, 160, 150, 140, 130, 120, 110, 100, 90, 80, 70, 60, 50, 49, 48, 47, 46, 45, 44, 43, 42, 41, 40, 39, 38, 37, 36, 35, 34, 33, 32, 31, 30, 29, 28, 27, 26, 25, 24, 23, 22, 21, or 20 basepairs in length. In some embodiments, the 5′ homology arm is at least 20 basepairs in length. In some embodiments, the 5′ homology arm is at least 40 basepairs in length. In some embodiments, the 5′ homology arm is at least 50 basepairs in length. In some embodiments, the 5′ homology arm is at least 70 basepairs in length. In some embodiments, the 5′ homology arm is at least 100 basepairs in length. In some embodiments, the 5′ homology arm is at least 200 basepairs in length. In some embodiments, the 5′ homology arm is at least 300 basepairs in length. In some embodiments, the 5′ homology arm is at least 400 basepairs in length. In some embodiments, the 5′ homology arm is at least 500 basepairs in length. In some embodiments, the 5′ homology arm is at least 600 basepairs in length. In some embodiments, the 5′ homology arm is at least 700 basepairs in length. In some embodiments, the 5′ homology arm is at least 1000 basepairs in length. In some embodiments, the 5′ homology arm is at least 1500 basepairs in length. In some embodiments, the 5′ homology arm is at least 2000 basepairs in length. In some embodiments, the 5′ homology arm is about 20 basepairs in length. In some embodiments, the 5′ homology arm is about 40 basepairs in length. In some embodiments, the 5′ homology arm is 250 basepairs in length or less. In some embodiments, the 5′ homology arm is about 100 basepairs in length. In some embodiments, the 5′ homology arm is about 200 basepairs in length.

In some embodiments, the 3′ homology arm is between 50 to 250 basepairs in length. In some embodiments, the 3′ homology arm is between 50-2000 basepairs in length. In some embodiments, the 3′ homology arm is between 50-1500 basepairs in length. In some embodiments, the 3′ homology arm is between 50-1000 basepairs in length. In some embodiments, the 3′ homology arm is between 50-500 basepairs in length. In some embodiments, the 3′ homology arm is between 150 basepairs to 250 basepairs in length. In some embodiments, the 3′ homology arm is 2000 basepairs or less in length. In some embodiments, the 3′ homology arm is 1500 basepairs or less in length. In some embodiments, the 3′ homology arm is 1000 basepairs or less in length. In some embodiments, the 3′ homology arm is 700 basepairs or less in length. In some embodiments, the 3′ homology arm is 650 basepairs or less in length. In some embodiments, the 3′ homology arm is 600 basepairs or less in length. In some embodiments, the 3′ homology arm is 550 basepairs or less in length. In some embodiments, the 3′ homology arm is 500 basepairs or less in length. In some embodiments, the 3′ homology arm is 400 basepairs or less in length. In some embodiments, the 3′ homology arm is 300 basepairs or less in length. In some embodiments, the 3′ homology arm is 250 basepairs or less in length. In some embodiments, the 3′ homology arm is 200 basepairs or less in length. In some embodiments, the 3′ homology arm is 150 basepairs or less in length. In some embodiments, the 3′ homology arm is less than 100 basepairs in length. In some embodiments, the 3′ homology arm is 50 basepairs in length or less. In some embodiments, the 3′ homology arm is 250, 200, 190, 180, 170, 160, 150, 140, 130, 120, 110, 100, 90, 80, 70, 60, 50, 49, 48, 47, 46, 45, 44, 43, 42, 41, 40, 39, 38, 37, 36, 35, 34, 33, 32, 31, 30, 29, 28, 27, 26, 25, 24, 23, 22, 21, or 20 basepairs in length. In some embodiments, the 3′ homology arm is at least 20 basepairs in length. In some embodiments, the 3′ homology arm is at least 40 basepairs in length. In some embodiments, the 3′ homology arm is at least 50 basepairs in length. In some embodiments, the 3′ homology arm is at least 70 basepairs in length. In some embodiments, the 3′ homology arm is at least 100 basepairs in length. In some embodiments, the 3′ homology arm is at least 200 basepairs in length. In some embodiments, the 3′ homology arm is at least 300 basepairs in length. In some embodiments, the 3′ homology arm is at least 400 basepairs in length. In some embodiments, the 3′ homology arm is at least 500 basepairs in length. In some embodiments, the 3′ homology arm is at least 600 basepairs in length. In some embodiments, the 3′ homology arm is at least 700 basepairs in length. In some embodiments, the 3′ homology arm is at least 1000 basepairs in length. In some embodiments, the 3′ homology arm is at least 1500 basepairs in length. In some embodiments, the 3′ homology arm is at least 2000 basepairs in length. In some embodiments, the 3′ homology arm is about 20 basepairs in length. In some embodiments, the 3′ homology arm is about 40 basepairs in length. In some embodiments, the 3′ homology arm is 250 basepairs in length or less. In some embodiments, the 3′ homology arm is about 100 basepairs in length. In some embodiments, the 3′ homology arm is about 200 basepairs in length. In some embodiments, the 3′ homology arm is 250 basepairs in length or less. In some embodiments, the 3′ homology arm is 200 basepairs in length or less. In some embodiments, the 3′ homology arm is 150 basepairs in length or less. In some embodiments, the 3′ homology arm is 100 basepairs in length or less. In some embodiments, the 3′ homology arm is 50 basepairs in length or less. In some embodiments, the 3′ homology arm is 250, 200, 190, 180, 170, 160, 150, 140, 130, 120, 110, 100, 90, 80, 70, 60, 50, 49, 48, 47, 46, 45, 44, 43, 42, 41, 40, 39, 38, 37, 36, 35, 34, 33, 32, 31, 30, 29, 28, 27, 26, 25, 24, 23, 22, 21, or 20 basepairs in length. In some embodiments, the 3′ homology arm is 40 basepairs in length.

The 5′ and 3′ homology arms can be of the same length or can differ in length. In some embodiments, the 5′ and 3′ homology arms are amplified to allow for the quantitative assessment of gene editing events, such as targeted integration, at a target nucleic acid. In some embodiments, the quantitative assessment of the gene editing events may rely on the amplification of both the 5′ junction and 3′ junction at the site of targeted integration by amplifying the whole or a part of the homology arm using a single pair of PCR primers in a single amplification reaction. Accordingly, although the length of the 5′ and 3′ homology arms may differ, the length of each homology arm should be capable of amplification (e.g., using PCR), as desired. Moreover, when amplification of both the 5′ and the difference in lengths of the 5′ and 3′ homology arms in a single PCR reaction is desired, the length difference between the 5′ and 3′ homology arms should allow for PCR amplification using a single pair of PCR primers.

In some embodiments, the length of the 5′ and 3′ homology arms does not differ by more than 75 nucleotides. Thus, in some embodiments, when the 5′ and 3′ homology arms differ in length, the length difference between the homology arms is less than 70, 60, 50, 40, 30, 20, 10, 9, 8, 7, 6, 5, 4, 3, 2, 1 nucleotides or base pairs. In some embodiments, the 5′ and 3′ homology arms differ in length by at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, or 75 nucleotides. In some embodiments, the length difference between the 5′ and 3′ homology arms is less than 70, 60, 50, 40, 30, 20, 10, 9, 8, 7, 6, 5, 4, 3, 2, 1 base pairs. In some embodiments, the 5′ and 3′ homology arms differ in length by at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, or 75 base pairs.

Donor templates of the disclosure are designed to facilitate homologous recombination with a target nucleic acid having a cleavage site, wherein the target nucleic acid comprises, from 5′ to 3′,
P1--H1--X--H2--P2,
wherein P1 is a first priming site; H1 is a first homology arm; X is the cleavage site; H2 is a second homology arm; and P2 is a second priming site; and wherein the donor template comprises, from 5′ to 3′,
A1--P2′--N--A2, or A1--N--P1′--A2,
wherein A1 is a homology arm that is substantially identical to H1; P2′ is a priming site that is substantially identical to P2; N is a cargo; P1′ is a priming site that is substantially identical to P1; and A2 is a homology arm that is substantially identical to H2. In one embodiment, the target nucleic acid is double stranded. In one embodiment, the target nucleic acid comprises a first strand and a second strand. In another embodiment, the target nucleic acid is single stranded. In one embodiment, the target nucleic acid comprises a first strand.

In some embodiments, the donor template comprises, from 5′ to 3′,
A1--P2′--N--A2.

In some embodiments, the donor template comprises, from 5′ to 3′,
A1--P2′--N--P1′--A2.

In some embodiments, the target nucleic acid comprises, from 5′ to 3′,
P1--H1--X--H2--P2,
wherein P1 is a first priming site; H1 is a first homology arm; X is the cleavage site; H2 is a second homology arm; and P2 is a second priming site; and the first strand of the donor template comprises, from 5′ to 3′,
A1--P2′--N--A2, or A1--N--P1′--A2,
wherein A1 is a homology arm that is substantially identical to H1; P2′ is a priming site that is substantially identical to P2; N is a cargo; P1′ is a priming site that is substantially identical to P1; and A2 is a homology arm that is substantially identical to H2.

In some embodiments, a first strand of the donor template comprises, from 5′ to 3′,
A1--P2′--N--P1′--A2.

In some embodiments, a first strand of the donor template comprises, from 5′ to 3′,
A1--N--P1′--A2.

In some embodiments, A1 is 700 basepairs or less in length. In some embodiments, A1 is 650 basepairs or less in length. In some embodiments, A1 is 600 basepairs or less in length. In some embodiments, A1 is 550 basepairs or less in length. In some embodiments, A1 is 500 basepairs or less in length. In some embodiments, A1 is 400 basepairs or less in length. In some embodiments, A1 is 300 basepairs or less in length. In some embodiments, A1 is less than 250 base pairs in length. In some embodiments, A1 is less than 200 base pairs in length. In some embodiments, A1 is less than 150 base pairs in length. In some embodiments, A1 is less than 100 base pairs in length. In some embodiments, A1 is less than 50 base pairs in length. In some embodiments, the A1 is 250, 200, 190, 180, 170, 160, 150, 140, 130, 120, 110, 100, 90, 80, 70, 60, 50, 49, 48, 47, 46, 45, 44, 43, 42, 41, 40, 39, 38, 37, 36, 35, 34, 33, 32, 31, 30, 29, 28, 27, 26, 25, 24, 23, 22, 21, or 20 base pairs in length. In some embodiments, A1 is 40 base pairs in length. In some embodiments, A1 is 30 base pairs in length. In some embodiments, A1 is 20 base pairs in length.

In some embodiments, A2 is 700 basepairs or less in length. In some embodiments, A2 is 650 basepairs or less in length. In some embodiments, A2 is 600 basepairs or less in length. In some embodiments, A2 is 550 basepairs or less in length. In some embodiments, A2 is 500 basepairs or less in length. In some embodiments, A2 is 400 basepairs or less in length. In some embodiments, A2 is 300 basepairs or less in length. In some embodiments, A2 is less than 250 base pairs in length. In some embodiments, A2 is less than 200 base pairs in length. In some embodiments, A2 is less than 150 base pairs in length. In some embodiments, A2 is less than 100 base pairs in length. In some embodiments, A2 is less than 50 base pairs in length. In some embodiments, A2 is 250, 200, 190, 180, 170, 160, 150, 140, 130, 120, 110, 100, 90, 80, 70, 60, 50, 49, 48, 47, 46, 45, 44, 43, 42, 41, 40, 39, 38, 37, 36, 35, 34, 33, 32, 31, 30, 29, 28, 27, 26, 25, 24, 23, 22, 21, or 20 base pairs in length. In some embodiments, A2 is 40 base pairs in length. In some embodiments, A2 is 30 base pairs in length. In some embodiments, A2 is 20 base pairs in length.

In some embodiments, A1 is 700 nucleotides or less in length. In some embodiments, A1 is 650 nucleotides or less in length. In some embodiments, A1 is 600 nucleotides or less in length. In some embodiments, A1 is 550 nucleotides or less in length. In some embodiments, A1 is 500 nucleotides or less in length. In some embodiments, A1 is 400 nucleotides or less in length. In some embodiments, A1 is 300 nucleotides or less in length. In some embodiments, A1 is less than 250 nucleotides in length. In some embodiments, A1 is less than 200 nucleotides in length. In some embodiments, A1 is less than 150 nucleotides in length. In some embodiments, A1 is less than 100 nucleotides in length. In some embodiments, A1 is less than 50 nucleotides in length. In some embodiments, the A1 is 250, 200, 190, 180, 170, 160, 150, 140, 130, 120, 110, 100, 90, 80, 70, 60, 50, 49, 48, 47, 46, 45, 44, 43, 42, 41, 40, 39, 38, 37, 36, 35, 34, 33, 32, 31, 30, 29, 28, 27, 26, 25, 24, 23, 22, 21, or 20 nucleotides in length. In some embodiments, A1 is at least 40 nucleotides in length. In some embodiments, A1 is at least 30 nucleotides in length. In some embodiments, A1 is at least 20 nucleotides in length.

In some embodiments, A2 is 700 nucleotides or less in length. In some embodiments, A2 is 650 basepairs or less in length. In some embodiments, A2 is 600 nucleotides or less in length. In some embodiments, A2 is 550 nucleotides or less in length. In some embodiments, A2 is 500 nucleotides or less in length. In some embodiments, A2 is 400 nucleotides or less in length. In some embodiments, A2 is 300 nucleotides or less in length. In some embodiments, A2 is less than 250 nucleotides in length. In some embodiments, A2 is less than 200 nucleotides in length. In some embodiments, A2 is less than 150 nucleotides in length. In some embodiments, A2 is less than 100 nucleotides in length. In some embodiments, A2 is less than 50 nucleotides in length. In some embodiments, A2 is 250, 200, 190, 180, 170, 160, 150, 140, 130, 120, 110, 100, 90, 80, 70, 60, 50, 49, 48, 47, 46, 45, 44, 43, 42, 41, 40, 39, 38, 37, 36, 35, 34, 33, 32, 31, 30, 29, 28, 27, 26, 25, 24, 23, 22, 21, or 20 nucleotides in length. In some embodiments, A2 is at least 40 nucleotides in length. In some embodiments, A2 is at least 30 nucleotides in length. In some embodiments, A2 is at least 20 nucleotides in length.

In some embodiments, the nucleic acid sequence of A1 is substantially identical to the nucleic acid sequence of H1. In some embodiments A1 has a sequence that is identical to, or differs by no more than 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 nucleotides from H1. In some embodiments A1 has a sequence that is identical to, or differs by no more than 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 base pairs from H1.

In some embodiments, the nucleic acid sequence of A2 is substantially identical to the nucleic acid sequence of H2. In some embodiments A2 has a sequence that is identical to, or differs by no more than 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 nucleotides from H2. In some embodiments A2 has a sequence that is identical to, or differs by no more than 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 base pairs from H2.

Whatever format is used, a donor template can be designed to avoid undesirable sequences. In certain embodiments, one or both homology arms can be shortened to avoid overlap with certain sequence repeat elements, e.g., Alu repeats, LINE elements, etc.

B. Priming Sites

The donor templates described herein comprise at least one priming site having a sequence that is substantially similar to, or identical to, the sequence of a priming site within the target nucleic acid, but is in a different spatial order or orientation relative to a homology sequence/homology arm in the donor template. When the donor template is homologously recombined with the target nucleic acid, the priming site(s) are advantageously incorporated into the target nucleic acid, thereby allowing for the amplification of a portion of the altered nucleic acid sequence that results from the recombination event. In some embodiments, the donor template comprises at least one priming site. In some embodiments, the donor template comprises a first and a second priming site. In some embodiments, the donor template comprises three or more priming sites.

In some embodiments, the donor template comprises a priming site P1′, that is substantially similar or identical to a priming site, P1, within the target nucleic acid, wherein upon integration of the donor template at the target nucleic acid, P1′, is incorporated downstream from P1. In some embodiments, the donor template comprises a first priming site, P1′, and a second priming site, P2′; wherein, P1′, is substantially similar or identical to a first priming site, P1, within the target nucleic acid; wherein P2′ is substantially similar or identical to second priming site, P2, within the target nucleic acid; and wherein P1 and P2 are not substantially similar or identical. In some embodiments, the donor template comprises a first priming site, P1′, and a second priming site, P2′; wherein, P1′, is substantially similar or identical to a first priming site. P1, within the target nucleic acid; wherein P2′ is substantially similar or identical to second priming site. P2, within the target nucleic acid; wherein P2 is located downstream from P1 on the target nucleic acid; wherein P1 and P2 are not substantially similar or identical; and wherein upon integration of the donor template at the target nucleic acid, P1′, is incorporated downstream from P1. P2′ is incorporated upstream from P2, and P2′ is incorporated upstream from P1.

In some embodiments, the target nucleic acid comprises a first priming site (P1) and a second priming site (P2). The first priming site in the target nucleic acid may be within the first homology arm. Alternatively, the first priming site in the target nucleic acid may be 5′ and adjacent to the first homology arm. The second priming site in the target nucleic acid may be within the second homology arm. Alternatively, the second priming site in the target nucleic acid may be 3′ and adjacent to the second homology arm.

The donor template may comprise a cargo sequence, a first priming site (P1′), and a second priming site (P2′), wherein P2′ is located 5′ from the cargo sequence, wherein P1′ is located 3′ from the cargo sequence (i.e., A1--P2′--N--P1′--A2), wherein P1′ is substantially identical to PI, and wherein P2′ is substantially identical to P2. In this scenario, a primer pair comprising an oligonucleotide targeting P1′ and P1 and an oligonucleotide comprising P2′ and P2 may be used to amplify the targeted locus, thereby generation three amplicons of similar size which may be sequenced to determine whether targeted integration has occurred. The first amplicon, Amplicon X, results from the amplification of the nucleic acid sequence between P1 and P2 as a result of non-targeted integration at the target nucleic acid. The second amplicon, Amplicon Y, results from the amplification of the nucleic acid sequence between P1 and P2′ following a targeted integration event at the target nucleic acid, thereby amplifying the 5′ junction. The third amplicon, Amplicon Z, results from the amplification of the nucleic acid sequence between P1′ and P2 following a targeted integration event at the target nucleic acid, thereby amplifying the 3′ junction. In other embodiments, P1′ may be identical to P1. Moreover, P2′ may be identical to P2.

In some embodiments, the donor template comprises a cargo and a priming site (P1′), wherein P1′ is located 3′ from the cargo nucleic acid sequence (i.e., A1--N--P1′--A2) and P1′ is substantially identical to P1. In this scenario, a primer pair comprising an oligonucleotide targeting P1′ and P1 and an oligonucleotide targeting P2 may be used to amplify the targeted locus, thereby generation two amplicons of similar size which may be sequenced to determine whether targeted integration has occurred. The first amplicon, Amplicon X, results from the amplification of the nucleic acid sequence between P1 and P2 as a result of non-targeted integration at the target nucleic acid. The second amplicon, Amplicon Z, results from the amplification of the nucleic acid sequence between P1′ and P2 following a targeted integration event at the target nucleic acid, thereby amplifying the 3′ junction. In other embodiments, P1′ may be identical to P1. Moreover, P2′ may be identical to P2.

In some embodiments, the target nucleic acid comprises a first priming site (P1) and a second priming site (P2), and the donor template comprises a priming site P2′, wherein P2′ is located 5′ from the cargo nucleic acid sequence (i.e., A1--P2′--N--A2), and P2′ is substantially identical to P2. In this scenario, a primer pair comprising an oligonucleotide targeting P2′ and P2 and an oligonucleotide targeting PI may be used to amplify the targeted locus, thereby generation two amplicons of similar size which may be sequenced to determine whether targeted integration has occurred. The first amplicon, Amplicon X, results from the amplification of the nucleic acid sequence between P1 and P2 as a result of non-targeted integration at the target nucleic acid. The second amplicon, Amplicon Y, results from the amplification of the nucleic acid sequence between P1 and P2′ following a targeted integration event at the target nucleic acid, thereby amplifying the 5′ junction. In other embodiments, P1′ may be identical to P1. Moreover, P2′ may be identical to P2.

A priming site of the donor template may be of any length that allows for the quantitative assessment of gene editing events at a target nucleic acid by amplification and/or sequencing of a portion of the target nucleic acid. For example, in some embodiments, the target nucleic acid comprises a first priming site (P1) and the donor template comprises a priming site (P1′). In these embodiments, the length of the P1′ priming site and the P1 primer site is such that a single primer can specifically anneal to both priming sites (for example, in some embodiments, the length of the P1′ priming site and the P1 priming site is such that both have the same or very similar GC content).

In some embodiments, the priming site of the donor template is 60 nucleotides in length. In some embodiments, the priming site of the donor template is less than 60 nucleotides in length. In some embodiments, the priming site of the donor template is less than 50 nucleotides in length. In some embodiments, the priming site of the donor template is less than 40 nucleotides in length. In some embodiments, the priming site of the donor template is less than 30 nucleotides in length. In some embodiments the priming site of the donor template is 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59 or 60 nucleotides in length. In some embodiments, the priming site of the donor template is 60 base pairs in length. In some embodiments, the priming site of the donor template is less than 60 base pairs in length. In some embodiments, the priming site of the donor template is less than 50 base pairs in length. In some embodiments, the priming site of the donor template is less than 40 base pairs in length. In some embodiments, the priming site of the donor template is less than 30 base pairs in length. In some embodiments the priming site of the donor template is 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59 or 60 base pairs in length.

In some embodiments, upon resolution of the cleavage event at the cleavage site in the target nucleic acid and homologous recombination of the donor template with the target nucleic acid, the distance between the first priming site of the target nucleic acid (P1) and now integrated P2′ priming site is 600 base pairs or less. In some embodiments, upon resolution of the cleavage event and homologous recombination of the donor template with the target nucleic acid, the distance between the first priming site of the target nucleic acid (P1) and now integrated P2′ priming site is 550, 500, 450, 400, 350, 300, 250, 200, 150 base pairs or less. In some embodiments, upon resolution of the cleavage event at the target nucleic acid and homologous recombination of the donor template with the target nucleic acid, the distance between the first priming site of the target nucleic acid (P1) and now integrated P2′ priming site is 600 nucleotides or less. In some embodiments, upon resolution of the cleavage event at the target nucleic acid and homologous recombination of the donor template with the target nucleic acid, the distance between the first priming site of the target nucleic acid (P1) and now integrated P2′ priming site is 550, 500, 450, 400, 350, 300, 250, 200, 150 nucleotides or less.

In some embodiments, the target nucleic acid comprises a second priming site (P2) and the donor template comprises a priming site (P2′) that is substantially identical to P2. In some embodiments, upon resolution of the cleavage event at the target nucleic acid and homologous recombination of the donor template with the target nucleic acid, the distance between the second priming site of the target nucleic acid (P2) and now integrated P1′ priming site is 600 base pairs or less. In some embodiments, upon resolution of the cleavage event at the target nucleic acid and homologous recombination of the donor template with the target nucleic acid, the distance between the second priming site of the target nucleic acid (P2) and now integrated P1′ priming site is 550, 500, 450, 400, 350, 300, 250, 200, 150 base pairs or less. In some embodiments, upon resolution of the cleavage event at the target nucleic acid and homologous recombination of the donor template with the target nucleic acid, the distance between the second priming site of the target nucleic acid (P2) and now integrated P1′ priming site is 600 nucleotides or less. In some embodiments, upon resolution of the cleavage event at the target nucleic acid and homologous recombination of the donor template with the target nucleic acid, the distance between the second priming site of the target nucleic acid (P2) and now integrated P1′ priming site is 550, 500, 450, 400, 350, 300, 250, 200, 150 nucleotides or less.

In some embodiments, the nucleic acid sequence of P2′ is comprised within the nucleic acid sequence of A1. In some embodiments, the nucleic acid sequence of P2′ is immediately adjacent to the nucleic acid sequence of A1. In some embodiments, the nucleic acid sequence of P2′ is immediately adjacent to the nucleic acid sequence of N. In some embodiments, the nucleic acid sequence of P2′ is comprised within the nucleic acid sequence of N.

In some embodiments, the nucleic acid sequence of P1′ is comprised within the nucleic acid sequence of A2. In some embodiments, the nucleic acid sequence of P1′ is immediately adjacent to the nucleic acid sequence of A2. In some embodiments, the nucleic acid sequence of P1′ is immediately adjacent to the nucleic acid sequence of N. In some embodiments, the nucleic acid sequence of P1′ is comprised within the nucleic acid sequence of N.

In some embodiments, the nucleic acid sequence of P2′ is comprised within the nucleic acid sequence of S1. In some embodiments, the nucleic acid sequence of P2′ is immediately adjacent to the nucleic acid sequence of S1. In some embodiments, the nucleic acid sequence of P1′ is comprised within the nucleic acid sequence of S2. In some embodiments, the nucleic acid sequence of P1′ is immediately adjacent to the nucleic acid sequence of S2.

C. Cargo

The donor template of the gene editing systems described herein comprises a cargo (N). The cargo may be of any length necessary in order to achieve the desired outcome. For example, a cargo sequence may be less than 2500 base pairs or less than 2500 nucleotides in length. In other embodiments, the cargo sequence may be 12 kb or less. In other embodiments, the cargo sequence may be 10 kb or less. In other embodiments, the cargo sequence may be 7 kb or less. In other embodiments, the cargo sequence may be 5 kb or less. In other embodiments, the cargo sequence may be 4 kb or less. In other embodiments, the cargo sequence may be 3 kb or less. In other embodiments, the cargo sequence may be 2 kb or less. In other embodiments, the cargo sequence may be 1 kb or less. In one embodiment, the cargo can be between about 5-10 kb in length. In another embodiment, the cargo can be between about 1-5 kb in length. In another embodiment, the cargo can be between about 0-1 kb in length. For example, in exemplary embodiments, the cargo can be about 1000, 900, 800, 700, 600, 500, 400, 300, 200, or 100 base pairs or nucleotides in length. In other exemplary embodiments, the cargo can be about 100, 90, 80, 70, 60, 50, 40, 30, 20, 10, 9, 8, 7, 6, 5, 4, 3, 2, 1 or 0 base pairs or nucleotides in length. Those of skill in the art will readily ascertain that when the donor template is delivered using a delivery vehicle (e.g., a viral delivery vehicle such as an adeno-associated virus (AAV), adenovirus, lentivirus, integration-deficient lentivirus (IDLV), or herpes simplex virus (HSV) delivery vehicle) with size limitations, the size of the donor template, including cargo, should not exceed the size limitation of the delivery system.

In some embodiments, the cargo comprises a replacement sequence. In some embodiments, the cargo comprises an exon of a gene sequence. In some embodiments, the cargo comprises an intron of a gene sequence. In some embodiments, the cargo comprises a cDNA sequence. In some embodiments, the cargo comprises a transcriptional regulatory element. In some embodiments, the cargo comprises a reverse complement of a replacement sequence, an exon of a gene sequence, an intron of a gene sequence, a cDNA sequence or a transcriptional regulatory element. In some embodiments, the cargo comprises a portion of a replacement sequence, an exon of a gene sequence, an intron of a gene sequence, a cDNA sequence or a transcriptional regulatory element. In some embodiments, the cargo is a transgene sequence. In some embodiments, the cargo introduces a deletion into a target nucleic acid. In some embodiments, the cargo comprises an exogenous sequence. In other embodiments, the cargo comprises an endogenous sequence.

Replacement sequences in donor templates have been described elsewhere, including in Cotta-Ramusino et al. A replacement sequence can be any suitable length (including zero nucleotides, where the desired repair outcome is a deletion), and typically includes one, two, three or more sequence modifications relative to the naturally-occurring sequence within a cell in which editing is desired. One common sequence modification involves the alteration of the naturally-occurring sequence to repair a mutation that is related to a disease or condition of which treatment is desired. Another common sequence modification involves the alteration of one or more sequences that are complementary to, or code for, the PAM sequence of the RNA-guided nuclease or the targeting domain of the gRNA(s) being used to generate an SSB or DSB, to reduce or eliminate repeated cleavage of the target site after the replacement sequence has been incorporated into the target site.

Specific cargo can be selected for a given application based on the cell type to be edited, the target nucleic acid, and the effect to be achieved.

For example, it may be desirable, in some embodiments, to “knock in” a desired gene sequence at a selected chromosomal locus in a target cell. In such cases, the cargo can comprise the desired gene sequence. In some embodiments, the gene sequence encodes a desired protein, e.g., an exogenous protein, an orthologous protein, or an endogenous protein, or a combination thereof.

In embodiments, the cargo can contain a wild-type sequence, or a sequence comprising one or more modifications with respect to a wild-type sequence. For example, in embodiments in which it is desirable to correct a mutation in a target gene in a cell, the cargo can be designed to restore the wild-type sequence to the target protein.

It may also be desirable, in other embodiments, to “knock out” a gene sequence at a selected chromosomal locus in the target cell. In such cases, the cargo can be designed to integrate at site that disrupts expression of the target gene sequence, for example, at a coding region of the target gene sequence, or at an expression control region for the target gene sequence, e.g., a promoter or enhancer of the target gene sequence. In other embodiments, the cargo can be designed to disrupt the target gene sequence. For example, in some embodiments, the cargo can introduce a deletion, insertion, stop codon, or frameshift mutation into the target nucleic acid.

In some embodiments, the donor is designed to delete all or a portion of the target nucleic acid sequence. In such embodiments, the homology arms of the donor can be designed to flank the desired deletion site. In some embodiments, the donor does not contain a cargo sequence between the homology arms, resulting in a deletion of the portion of the target nucleic acid positioned between the homology arms following targeted integration of the donor. In other embodiments, the donor contains a cargo sequence homologous to the target nucleic acid in which one or more nucleotides of the target nucleic acid sequence are absent from the cargo. Following targeted integration of the donor, the target nucleic acid will comprise a deletion at the residues absent from the cargo sequence. The size of the deletion can be selected based on the size of the target nucleic acid and the desired effect. In one embodiment, the donor is designed to introduce a deletion of 1-2000 nucleotides in the target nucleic acid following targeted integration. In other embodiments, the donor is designed to introduce a deletion of 1-1000 nucleotides in the target nucleic acid following targeted integration. In other embodiments, the donor is designed to introduce a deletion of 1-500 nucleotides in the target nucleic acid following targeted integration. In other embodiments, the donor is designed to introduce a deletion of 1-100 nucleotides in the target nucleic acid following targeted integration. In exemplary embodiments, the donor is designed to introduce a deletion of about 2000, 1500, 1000, 900, 800, 700, 600, 500, 400, 300, 200, 100, 90, 80, 70, 60, 50, 40, 30, 20, 15, 10, 9, 8, 7, 6, 5, 4, 3, 2, or 1 nucleotides in the target nucleic acid following targeted integration. In other embodiments, the donor is designed to introduce a deletion of more than 2000 nucleotides from the target nucleic acid following targeted integration, for example, a deletion of about 2000, 3000, 4000, 5000, 6000, 7000, 8000, 9000, 10,000 nucleotides or more.

In some embodiments, the cargo can comprise a promoter sequence. In other embodiments, the cargo is designed to integrate at a site that is under the control of a promoter endogenous to the target cell.

In one embodiment, a cargo encoding an exogenous or orthologous protein or polypeptide can be integrated into a chromosomal sequence encoding a protein, such that the chromosomal sequence is inactivated, but the exogenous sequence is expressed. In other embodiments, the cargo sequence may be integrated into a chromosomal sequence without altering expression of a chromosomal sequence. This can be achieved by integrating the cargo at a “safe harbor” locus, such as the Rosa26 locus, HPRT locus, or AAV locus.

In some embodiments, the cargo encodes a protein related to a disease or disorder. In one embodiment, the cargo can encode a wild-type form of a protein, or is designed to restore expression of a wild-type form of a protein, where the protein is deficient in a subject afflicted with a disease or disorder. In other embodiments, the cargo encodes a protein related to a disease or disorder, where the protein encoded by the cargo comprises at least one modification, such that the altered version of the protein protects against the development of the disease or disorder. In other embodiments, the cargo encodes a protein comprising at least one modification, such that the altered version of the protein causes or potentiates a disease or disorder.

In some embodiments, the cargo can be used to insert a gene from one species into the genome of a different species. For example, “humanized” animal models and/or “humanized” animal cells can be generated through targeted integration of human genes into the genome of a non-human animal species, e.g., mouse, rat, or non-human primate species. In one embodiment, such humanized animal models and animal cells contain an integrated sequence encoding one or more human proteins.

In another embodiment, the cargo encodes a protein that confers a benefit on plant species, including crops such as grains, fruits, or vegetables. For example, the cargo can encode a protein that allows plants to be cultivated at higher temperatures, have a prolonged shelf life following harvest, or conver disease resistance. In some embodiments, the cargo can encode a protein that confers resistance to diseases or pests (see, e.g., Jones et al. (1994) Science 266:789 (cloning of the tomato Cf-9 gene for resistance to Cladosporium fulvum); Martin et al. (1993) Science 262:1432; Mindrinos et al. (1994) Cell 78:1089 (RSP2 gene for resistance to Pseudomonas syringae); PCT International Patent Publication No. WO 96/30517 (resistance to soybean cyst nematode)). In other embodiments, the cargo can encode a protein that encodes resistance to an herbicide, as described in US2013/0326645A1, the entire contents of which are incorporated herein by reference. In another embodiment, the cargo encodes a protein that confers a value-added trait to a plant cell, for example and without limitation: modified fatty acid metabolism, decreased phytate content, and modified carbohydrate composition effected, e.g., by transforming plants with a gene encoding an enzyme that alters the branching pattern of starch (See, e.g., Shiroza et al. (1988) J. Bacteol. 170:810 (nucleotide sequence of Streptococcus mutant fructosyltransferase gene); Steinmetz et al. (1985) Mol. Gen. Genet. 20:220 (levansucrase gene); Pen et al. (1992) Bio/Technology 10:292 (α-amylase); Elliot et al. (1993) Plant Molec. Biol. 21:515 (nucleotide sequences of tomato invertase genes); Sogaard et al. (1993) J. Biol. Chem. 268:22480 (barley α-amylase gene); and Fisher et al. (1993) Plant Physiol. 102:1045 (maize endosperm starch branching enzyme II)). Other exemplary cargo useful for targeted integration in plant cells are described in US2013/0326645A1, the entire contents of which are incorporated herein by reference.

Additional cargo can be selected by the skilled artisan for a given application based on the cell type to be edited, the target nucleic acid, and the effect to be achieved.

D. Stuffers

In some embodiments, the donor template may optionally comprise one or more stuffer sequences. Generally, a stuffer sequence is a heterologous or random nucleic acid sequence that has been selected to (a) facilitate (or to not inhibit) the targeted integration of a donor template of the present disclosure into a target site and the subsequent amplification of an amplicon comprising the stuffer sequence according to certain methods of this disclosure, but (b) to avoid driving integration of the donor template into another site. The stuffer sequence may be positioned, for instance, between a homology arm A1 and a primer site P2′ to adjust the size of the amplicon that will be generated when the donor template sequence is integrated into the target site. Such size adjustments may be employed, as one example, to balance the size of the amplicons produced by integrated and non-integrated target sites and, consequently to balance the efficiencies with which each amplicon is produced in a single PCR reaction; this in turn may facilitate the quantitative assessment of the rate of targeted integration based on the relative abundance of the two amplicons in a reaction mixture.

To facilitate targeted integration and amplification, the stuffer sequence may be selected to minimize the formation of secondary structures which may interfere with the resolution of the cleavage site by the DNA repair machinery (e.g., via homologous recombination) or which may interfere with amplification. In some embodiments, the donor template comprises, from 5′ to 3′,
A1--S1--P2′--N--A2, or
A1--N--P1′--S2--A2;

wherein S1 is a first stuffer sequence and S2 is a second stuffer sequence.

In some embodiments, the donor template comprises from 5′ to 3′,
A1--S1--P2′--N--P1′--S2--A2,
wherein S1 is a first stuffer sequence and S2 is a second stuffer sequence.

In some embodiments, the stuffer sequence comprises about the same guanine-cytosine content (“GC content”) as the genome of the cell as a whole. In some embodiments, the stuffer sequences comprises about the same GC content as the targeted locus. For example, when the target cell is a human cell, the stuffer sequence comprises about 40% GC content. In some embodiments, a stuffer sequence may be designed by generating random nucleic acid sequence sequences comprising the desired GC content. For example, to generate a stuffer sequence comprising 40% GC content, nucleic acid sequences having the following distribution of nucleotides may be designed: A=30%, T=30%, G=20%, C=20%. Methods for determining the GC content of the genome or the GC content of the target locus are known to those of skill in the art. Thus, in some embodiments, the stuffer sequence comprises 20%, 25%, 30%, 35%, 40%, 45%, 50%, 55% 60%, 65%, 70%, or 75% GC content. Exemplary 2.0 kilobase stuffer sequences having 40±5% GC content are provided in Table 2.

TABLE 2
Exemplary 2.0 Kilobase Stuffer Sequences Having 40 ± 5% GC Content

| SEQ | | |
|---|---|---|
| ID | GC | |
| NO. | Content | Stuffer Sequence |
| 1 | 38.40% | TCAATAGCCCAGTCGGTTTTGTTAGATACATTTTATCGAATCTGTAAAGATATTTT |
| | | ATAATAAGATAATATCAGCGCCTAGCTGCGGAATTCCACTCAGAGAATACCTCTCC |
| | | TGAATATCAGCCTTAGTGGCGTTATACGATATTTCACACTCTCAAAATCCCGAGTC |
| | | AGACTATACCCGCGCATGTTTAGTAAAGGTTGATTCTGAGATCTCGAGTCCAAAAA |
| | | AGATACCCACTACTTTAAAGATTTGCATTCAGTTGTTCCATCGGCCTGGGTAGTAA |
| | | AGGGGGTATGCTCGCTCCGAGTCGATGGAACTGTAAATGTTAGCCCTGATACGCGG |
| | | AACATATCAGTAACAATCTTTACCTAATATGGAGTGGGATTAAGCTTCATAGAGGA |
| | | TATGAAACGCTCGTAGTATGGCTTCCTACATAAGTAGAATTATTAGCAACTAAGAT |
| | | ATTACCACTGCCCAATAAAAGAGATTCCACTTAGATTCATAGGTAGTCCCAACAAT |
| | | CATGTCTGAATACTAAATTGATCAATTGGACTATGTCAAAATTATTTTGAAGAAGT |
| | | AATCATCAACTTAGGCGCTTTTTAGTGTTAAGAGCGCGTTATTGCCAACCGGGCTA |
| | | AACCTGTGTAACTCTTCAATATTGTATATAATTATAGGCAGAATAAGCTATGAGTG |
| | | CATTATGAGATAAACATAGATTTTTGTCCACTCGAAATATTTGAATTTCTTGATCC |
| | | TGGGCTAGTTCAGCCATAAGTTTTCACTAATAGTTAGGACTACCAATTACACTACA |
| | | TTCAGTTGCTGAAATTCACATCACTGCCGCAATATTTATGAAGCTATTATTGCATT |
| | | AAGACTTAGGAGATAAATACGAAGTTGATATATTTTTCAGAATCAGCGAAAAGACC |
| | | CCCTATTGACATTACGAATTCGAGTTTAACGAGCACATAAATCAAACACTACGAGG |
| | | TTACCAAGATTGTATCTTACATTAATGCTATCCAGCCAGCCGTCATGTTTAACTGG |
| | | ATAGTCATAATTAATATCCAATGATCGTTTCACGTAGCTGCATATCGAGGAAGTTG |
| | | TATAATTGAAAACCCACACATTAGAATGCATGGTGCATCGCTAGGGTTTATCTTAT |
| | | CTTGCTCGTGCCAAGAGTGTAGAAAGCCACATATTGATACGGAAGCTGCCTAGGAG |
| | | GTTGGTATATGTTGATTGTGCTCACCATCTCCCTTCCTAATCTCCTAGTGTTAAGT |
| | | CCAATCAGTGGGCTGGCTCTGGTTAAAAGTAATATACACGCTAGATCTCTCTACTA |
| | | TAATACAGGCTAAGCCTACGCGCTTTCAATGCACTGATTACCAACTTAGCTACGGC |
| | | CAGCCCCATTTAATGAATTATCTCAGATGAATTCAGACATTATTCTCTACAAGGAC |
| | | ACTTTAGAGTGTCCTGCGGAGGCATAATTATTATCTAAGATGGGGTAAGTCCGATG |
| | | GAAGACACAGATACATCGGACTATTCCTATTAGCCGAGAGTCAACCGTTAGAACTC |
| | | GGAAAAAGACATCGAAGCCGGTAACCTACGCACTATAAATTTCCGCAGAGACATAT |
| | | GTAAAGTTTTATTAGAACTGGTATCTTGATTACGATTCTTAACTCTCATACGCCGG |
| | | TCCGGAATTTGTGACTCGAGAAAATGTAATGACATGCTCCAATTGATTTCAAAATT |
| | | AGATTTAAGGTCAGCGAACTATGTTTATTCAACCGTTTACAACGCTATTATGCGCG |
| | | ATGGATGGGGCCTTGTATCTAGAAACCGAATAATAACATACCTGTTAAATGGCAAA |
| | | CTTAGATTATTGCGATTAATTCTCACTTCAGAGGGTTATCGTGCCGAATTCCTGAC |
| | | TTTGGAATAATAAAGTTGATATTGAGGTGCAATATCAACTACACTGGTTTAACCTT |
| | | TAAACACATGGAGTCAAGTTTTCGCTATGCCAGCCGGTTATGCAGCTAGGATTAAT |
| | | ATTAGAGCTCTTTTCTAATTCGTCCTAATAATCTCTTCAC |
| 2 | 38.90% | AAAACGTACTACGTCCACTAATATAGTGCTCAGGGCCTTTAAAGTTATGAACAGGA |
| | | ATACGGCGATGACGATAGAGATGTACAACTCAGTGCGAACCCCAGTGTATGTACAA |
| | | AAAGTTACTAATTCACTTTACTGTTTTGAGGATGTACCTGCCAAAAAGATTCAGAT |
| | | TATCAAAGTCAGATCTTTATATGACGGAACGCGCAAAGGATCCTATTAGGATGCGC |
| | | CTCAAAAAGCCATCTAAAAAGTTCATGTATTGAGCTTATTAGTAAAGGTATCAACA |
| | | AAAATGATTCCACCTTATATAAATAAGCTTGATCCCATTAATTGAATAATAAAGAC |
| | | CGAGTAATCACTTTTATGCATGTAACAAAAATCCCGTTTGCGGCTATGCTACAACG |
| | | GTCATCCCATAGAATATTATCATCGTACAAGCCCAAGACCCGATGCTCAACATTAG |
| | | AGCCAAATAACGTGCACACTCCTAATATGAGATGACTGCCGCTTTTAACACCAGAT |
| | | CTGTTAGTTAGGCCACGCACTTCCAAGTTTATCTAGAGTGCATGTCTTTATATATG |
| | | TTGGTCCCCTGTAATGACTTATAATATTTCCTTCGACTGTGTTGAACATCTGTAAC |
| | | AATAAAGACTAAAGCTCTGGGTATATAAGGTTGCAGTGGTACCTTATTAGGTCCAT |
| | | TATCGCAGAATACTGCGGATGGACAATCTTGCCAATTTAATTGACTATCTATTAGT |
| | | TTGCACAATATAACGATTCGTCTTGGACAAATTTGGCGAGTGAGCCCCTTACTCGC |
| | | TCAAAATGTTACAATTGCCGAGCTCGGAGTTGAATGATTAGTTACATATTATAGAA |
| | | CACAATGCAGATGTAGTTAGACAAGATGTGTTGATGAATGTCAAGTCTGACTGGAG |
| | | TAAAGGAACAAGAGCACCCACCTACGTATATTGCGCATTTTAAATGTAGCCTCGAC |
| | | TCTAACACGTGCGACGTGAGTCATAATTGTGCATGTTATTAGATCTATGGAATGTT |
| | | GTTTTTTTAATTATCAAACGTACGTCAAACCGCCAAACTCCGTGTGCCATAGAGTA |
| | | TACTCCTGAAGTTCGAAATTAGGCCATAAAGTCTTTCTTGCTGGTTGTGAAATGAA |
| | | GGGGTGTTTCATAATTTAACTTTGACTGCTTCTGTTGGGACGACGTACCCGTTCGT |
| | | TTGTTTGTCCTACTATTTAGTATCTTAAAACAGTCCATTTACCGTTAATGTTCTTA |
| | | ACCCTTAAAGATACAAACTTAGCTCTGTAATCAACTTCAAGACGTCTTTGACAGAA |
| | | CGTCTAAGACCCAGATCTGTGTTAGCCAACTCGTATTCAATTTCGTACCGGTGGAC |
| | | TTCGGCCCCTCACACTGCCATTAGTTGATGCTGAACTTTGTATTTGCTGGGTAGGA |
| | | TATATAACGATTTTGCAGATGTGTGTGCTAAGTATATTGTCTTAGTGACGGTCCAG |
| | | CATATAAAACACCTACACAAGAAGGTTATTCTTAATGGTTGATTGAATATTATTAA |
| | | ATTGTTGCTTTTACTTTTTCCTCCTACAAATTGTCATGAGCTCAAATTTGTTGACC |
| | | TAAGGTATTAATATTGTATCCTACACGGATTGTGAACGGTAGGGTCGTAACAATCG |
| | | TACTTTACGGCTTAAAAATTGTAAGCACCTTGCCAGGTAGATGAAAACTTAAAGGA |
| | | TAGAAGTATAGTAACTCACATGCTTGCGGCAGCATCGTAGGGCAGAGGTGTGATCT |
| | | TGGTGATTGAAATTAAGGGGTAGGATGATCGGCCGCATATATCGGCTACTAGGATT |
| | | AGATAGATGCAACGCTTTACTTTAATCAAGTGACGTCCGTATAAGTAAGACATCTA |
| | | ATGGCTGTATTTTTGTATACAAGTATAAGGAACCGGGGAGTCTTTATAGCGACGCG |
| | | TAATTATATATTCCAAATCAGTTAAGTGGCGTCGGTTACGAAACTAAAGAGAGTGT |
| | | TCAAGACGCAATGAAGAATCGTGAGCGTAATTGTTCGCGC |
| 3 | 39.30% | AACCCTCGTGTCCGGTAAAACACGCTTCGAATACAAAAGATTATATAGGTACGGAA |
| | | GGCTGGGAATCTTTCTTCGATGGAACTGAGATTATATTCCACTGTAACCTTATTAT |
| | | GACTATAGATTTCCAACATACGGATAGATTAATACCGACTGTAGATTCCATACTTG |
| | | AACTATGAAGCCGTACGAGTACCCATACTATAACTAAGACTATGACACGTGTGAAT |
| | | TCGTGTTTATCATAGTGCAAACTCTTGCTATTCCACATGGGAGTTTAGAACTCAGC |
| | | TGTTCCTATACAATTAGCACTACAAACCCACTAATATGGATAGCATGATACCATCT |
| | | GAGGAGGATTTGGTGTTACCATGTTGTAATCTAAGAAGTTTCACAAAATCAACGTT |
| | | AGATAAACGGCAATATACGCGCACTAATAATGAACCCCAAGATATCAGTTGAAAAA |
| | | TTTTCGATCTCCTCTTTAAATTAACAAATATTGCAGAGTAAGTACCGAAATTGTGA |
| | | CACAAGTGCCGTTTGCCCGTCTTTTTCACAGCCTATAAAGTTCAGATCTATATGGG |
| | | CTCCCACTTAACCTTCAGATAGATAACAAGTTACTGGAAGTGATTCTATCATAATA |
| | | CAATCAACTATAACACATCCAATGATATATCTCGAGAAAGTCGTAGTCTAGAGCTC |
| | | CTTCTATTATCCGGTCTTACCTAAATAGTTATATTTAGTTGCCCATTTAAAATTGG |
| | | ATAGGAGGAGGGGTGCTCATGATTTAAAAACCAACTGTGCATGCGGTTCTTTGATG |
| | | TGGATCCACCTTGCAAAGCGCTAAAGATAAAAGTAGTCACTACAGGAATTCAACTT |
| | | CCGTCGTTGTCAGCTGGCGCGGGAACCCATCTTGTGTAAAAAACTGTATAACCAGA |
| | | CACGTGGACTCGACCGAGAAACAGTCAGAACCTGTCACAAGAAATAATCTTGATTA |
| | | AAGGCTTTCACGGCAAACGGACCTCTTCCCTGCTGAAGTGTACGATTGAATATCCA |
| | | CATCGAAGGTCAATTACCCTCATCTTTTACATGGTCATAAGACAATAATCTCCTAT |
| | | TTGGATTAAAATCCGCGCACGAAAGATAAGAGTGGAATCGATTGCATTATCGAGTT |
| | | TTTAAGCCCCATACCCGACAGATGTGTAAAAAGTGTAGTGGTAATGGCGTCACCAA |
| | | GACCTATGCTTCTCATAATAATAGGACGTATGCCCTAGCTACTGCTAACGGTCGCT |
| | | CTTACAATACTAGCTAAAAGAAACAAATTTGAAAAGTTATGTAGGAAGTCATTGGC |
| | | GGTGAAAAAGTGAGAAAAAAGGTCCCCGGAGACTGTGCTTTCATGTTATCAAAGTA |
| | | CATGCCGAGTGAAGAGTTTGTTTTGATCAACTTTTATTATCTGGAGTCATTATACG |
| | | ATATTGCCATGGTTCCTTGGCTGTCCAACCAGGGGTCTTTTACACCAGATAATCTT |
| | | CTACTACACTACACCTCAGGTACGATTCTTTCGTTATCAATCGACTACAAGATTAT |
| | | AGTGTCTCTAAGGCGTGATGTAGGTTTTCCCTCAATGACAAAGACTTTACAGCAAT |
| | | CCGGTTCAATACGAGAATTAAGTGTGCGAGTAACAGCAAAGTAAAATCTAACAGAA |
| | | AGGAGACTCAGAAAACAACCTATTGAGGACTGTAATATCAACTCAGCATTATTGTT |
| | | TACTTTAAAATCTAATAATCGTTTCGAGGATATGAGCACGGTATCCTAACATCAAG |
| | | ACAAATACCACATCATCTAAATACAACTGGTTGCAATGAGTCGAATCGCGAACAAA |
| | | TAAAGCAACTATAAGCACGATAAACCACTGTTATGGGAATGATAAACAGTCTTATG |
| | | ACGTGGTCTATCTGTCGTAGGTGGTAAAGCCTTCTGAAGATCACTATCCAGTTCTG |
| | | GCCTCAAGAACCATTTAGACAGCCTTTTCTAAACATGATCGTTGCTATAAGGACCG |
| | | GGGACACCTAGACAAACTCACGGAAGGGATAACTTACATC |
| 4 | 38.90% | ACTGCTTATATAGGAGGTACAAACAGATACAATCCTTAGTTAACTAGAGAGAATGC |
| | | TTTTTTTCGACCGACACGCTTATAACTTCACTGGGCATGGTCACCATATTTAGGTA |
| | | AAACAAACTGCTGCGCTATATGTCGTACACATCCTGAGTGTACCAATATGTAGGTG |
| | | GAAGGCAAGTTCAATGAGACGTCAGTTACCAAGCAAATTTACATTCTAGCAGTTAT |
| | | AAATGTATTATGACGCAGTTCTTGTGGTGAGCGATCATTTACATTAAAACTTTATT |
| | | CAAGAGCGTATATTAGCATATATTTTCCGGAGAGTGCACTACGGGCCGAAATTTAG |
| | | GCTGGAACTCCGCAAATTGGTTACGACCCTGTATACATAGTTCTTATTATTAAGTA |
| | | AAATGTGTGAATAAAACCTACACGACGCGTCATATACGTAAAAGTTTATCTCTTGT |
| | | AGTAATCAACTAAATTAACTTACTACTATCTGGTCGTCCGTATGACCCTGTGAGCA |
| | | GATTATTTTCGACTCGACATCTATGAATTCTACGGCACGAAAAGTTGGTAACTTGT |
| | | ACTGGGTTAAACAATGTGTATTCGGGAGTCTGCGGAAGAACGTTTTTAATGTAACT |
| | | TCCTTTGCAAACCAAAATTTGGTCTATTCAAACTGACACTAGCGTAATCTATACCG |
| | | CATGAGATCCTGACATGATCCTATATCTATGCGCATAGGTACTCGCACCAATAAGT |
| | | GGGTCGTAGAATTTCACGTAACTCAATGTTGTCTCCTTTCATTTTTTGTTAATTCG |
| | | AGAAAACTACAAAAATAGTTAGTAAAATGCTCAAGGAGTCAGGTGCTACCTGTGGA |
| | | ATACATCTATGTCCAATGGAACTTGCTCCCTCGGATGTGCGATTTCGTTGTTCAGT |
| | | TGGGCCTTTAAGGAATACAGCAACTCCAACTCTTTGATTTTAGGTAAGTATTTGAT |
| | | TCGCGGAAAGTACAGTGTATAATCTGTTATTTGCCAAGACGTCATCGAAATCGAGT |
| | | GTATCGAGATCAGACCATCGCGCTATCGCAAGATATGAAGAGCATAGACAGATCAC |
| | | GATGCCAATCAGTGTCGATGGTGCGAAGACGCAGCCCCTGTGATCAAATCGTCCGT |
| | | TTCTCGATTTACTAGCGGAAAACAAAAACGAAGCGGTGAATACCCTGCGAGCTAAT |
| | | GTCTTTACCCGGTTATACGAGCTGATAACTCGGAAAATGCTAATATCGAGGCTGCG |
| | | CACTTAAAAAAATACTTTAATAATATTAATAAGCATAGCTGTATCATAACTTAAAA |
| | | TTCTACTGTATGATTTAGAATCTAACAGTGTTAACGATCTACAGACCGCACTAAGA |
| | | TGAAGACGGACTAATCTCCTCCCTAATTTTCCTTGTTGATTAGCAAAGGGAGATCC |
| | | TTTTGTTATTTGAGGTTTACGAGAAAGATGTAAGAGTCGAAATAATTACGTAAACC |
| | | TCATAGTCGTCACCTAGAGCAACTATAACATGAACCACTCGCCTTGGTTAAATATA |
| | | AAATAACTTCTTCTCTGTAACATTGTTGCACACAAGCGAGCGACAAAATTTCACAA |
| | | CATTTGTTGCGTAGATAATATTACTGCATCATTTTTGCGTCAGAGTGAATGTCACT |
| | | TATATAACTAGGAAAAATTAGTAGGATAGCTCTTGCGGTTGAGAGTAATGTCGACT |
| | | GAATCGACCGCCATAGATGGTAGAGGGAGTGATTCAAATAGATTAATGTATGCGCT |
| | | CCATCTATAAGGACGGACAAGGATCAATGTTCCCTTATACTTAGCTAACAGGACCC |
| | | TCTCCGAAGGTCTGATAATGCACTCATATAAGCATCGATGCGTCCTGAGTAGAAAA |
| | | ATCTTTACAAACTTTTAATAGATAAGTTATCTTGGAGGTGCTATCTATTCAAATCT |
| | | CTGAACAGATCTGCGGCATGATAATGTCTTTGTACCGGTGTGAATAATGTGAGTCA |
| | | GACGTCTGTGCGAAGTGGGAACCGAAATCTTTTAATCATT |
| 5 | 40.90% | GATTCGGTCGCGTTCCATAATCGAACCCTTAAGCCCATCTTCCAGCTGTTAACGTT |
| | | ATGTACCATCTTACCTCAATGTCAGCGATCTATGAGGTTCATGTTTTTGGTGGATT |
| | | AAAAAACTTCTTTATAGTGGTTTAGACAGAACGTTTAGCGCTGCGCTCGAAGTGTC |
| | | TTATCTAACGGAGGACTAAAATTACCTGGTCACTCCTTAGACTTTTCGTAGTACTT |
| | | AATTGCCGGACATCCGTTGGGCTACACCAGCAAGAACACAAAGTGGTATGTGTGAA |
| | | GCTAGACTGACCTCATGATTCGTACTACATTATAAGAATCAAGCTTCCCGGATTTG |
| | | TGTTCTGAGATATTACCACGTACATTTTTAAGGGGGTTCTTGACATCGTAACGCTA |
| | | AGGCTGATTAAAGAGGAGGGTGCTATGCAGAGTTTATTGGTGTTTCATCAATGTAT |
| | | CACACAAAATTAGCTACTATAGGAAGTAGCTTTGGTGCGAGCAGGGGGCGGTATGG |
| | | TTAAGAAAGCTATGGTAAGAAAGGCCCAGGTGATACTACGTGTAAGGTTGTGAAGA |
| | | GCCACAAGAGCCAAGTTTTGATATTCGACTTCCTCCGAATCTACAGCTTATCGAGG |
| | | GTTAAACGTTACGCATATTACGAGATTACATGATAGCTTCTCAGTTCTAGCACATT |
| | | TATGAGACCCTTTGAATGGTGTCAATAAATAGGAGGTCCCCATATGACAAGTAGAA |
| | | TACTAACTATAAGAGATTTGTAACGCTGGATACCATTTGCAGAGGATTGGCCCAAA |
| | | GAATGATTGCCCAACGCTTATATTGTCAGACCTTGCATTAGAAGAATAACGCAGAA |
| | | TACGACTGCAGTTTGATATAATTTTGGCTCTGGGTTGCCTTAGTATCATTACTAAT |
| | | AGACTTGTGGTCTATATCCATTTGTTTAATGGAATAGACTGGGTAAAACACACCTC |
| | | TTCCAGGCTGTAGTTCTTCATGTTGTAAGGATCCGTCATGGCGTGCAAACTAGGGG |
| | | AGGTATTTTTTGCTAATTGCGGTAACGGCTCCAGTTGGGATATCGTCAATATGTGC |
| | | CACTCGGCCCTTTCTCTGAGACGCTAAGATTTCCGTAAGGTATAGCGATAAGAGTC |
| | | TCTAATGCCAGAGGAATTGTTACCGCGAGCAAGATTCATGTCTATATATAAAATAT |
| | | CATCCACTTTGAATTACTGGTTGGAATCATCGTTCGCGTTATAACAAAAAACCTTT |
| | | TAATTATGTTACCACAGATCTCGAAGTCCCTTTTGAGGCAGAAGTTTAAATATAAG |
| | | CTCTAATTGTCGCATCTAACGGGTATATCGTCTCAACGGTAGGTCAAAAACATTTG |
| | | TTAACTTCAGACTGTACATTCGCATTTAACTCGCCATGTAAACCGCAATACATCTC |
| | | GTGCCTATCTCTCCTAGTAACGTATTATCGCTGGGTGAAAGCGCAACTAAGTAATA |
| | | AGTGAATGTCATTCACAATACCTAACTCTATCCGACGCGTAAGAGCGACCCAGCAG |
| | | TTTAATGACATGATAAATCAAATTCTATGCAAGGCAGTACTTGCTTTGTGGACGAT |
| | | AGCGATTTTCCACCGTATTGCGAAGTCAGTTATGCTGAAATTTTATTCCATTCGCA |
| | | TAACACCAAGGCTTACTCTTAGGAAAAAATGTAATACCGATTTTGGTATGAAGTAT |
| | | GTTACAGTACAGAATGAAATGCCCGGCGGCGTGGTCAAACTGTTTCCTGAGGTTCA |
| | | TATAGGGAAAGGTCATCCCTCAGAATTGGCCCCGTAATCGCAAAGCCTACGGGAGC |
| | | TTTCTTAAGTCCAACCGGTAAAGCCAAATCTCAATTCATATGAGGAAATGTTTGAC |
| | | CGATAAAGAATAGATTGTCGAACTAACAGTCACAGAGAAAATACGAGTAGCATCAC |
| | | CTAAACAAAGCAGGTAATAAAATAGACTAATGGAGATCATCGTATCGGCTTATGAC |
| | | CTGCGTCCATTTAAAGGCAATGAATACATTACCGACTAGA |
| 6 | 40.80% | AGTTATGAGGTTCACTTCTCATATAACACTATCAACAATGATCATCTCTTGCGAAA |
| | | CAAGCGCCCTACACAGCTTCAATGGAACCAAGAGCCATAATGAGGTAAGGGACGGC |
| | | TAGTTACTAATAAAGGAATCGATTTTACAAACACTAAATGAAAAACTTGCGCTGGT |
| | | TGCAATGCTATAAAAAAATGAAATGCAAACCAGTGAAGATCCCGATCAACCGTTCG |
| | | CTGATTTTTATTGATGCTGTACGTTGTGTTAGTTTAATGATATATAGGCCATCTCC |
| | | AGGTTACTTAGGACGCCAAAATTACTATTTTGAAGCTCAACCGTGGTATAATAGCT |
| | | ACAATAATTAATTGATGCCTGCAGGTCGTATCTCGAACGATTGTACGCATTACCTA |
| | | TGATATGAACAGAATCTGTATCCCATACTTAAAATCTTGACCTTGTAAAGATTTCG |
| | | CATACGCATTAAGAAATTTCGTTCTACCCGCACGGATTGTCCAAGTATATCTGGCC |
| | | ATTCACAGAAGTTACTAATCTTCATCTCTAAGTTTAAGGCCGACAAAGGGTCCAAA |
| | | ACCTGCGTAGGTTACAACGCAGCTTACACTCAGTGACTAACCAACGCTCAGTAGGG |
| | | TAACTGGACTTGTTCTCGCTATTCAGCTGGTACTGTAATGATCAACTTAGAACGGC |
| | | CCTATGGCTAAGCAAGGAGTACGCAATGTTTTAGAATACGTGTTTGCTCACACAGG |
| | | TAGTAGTTTAATATACCCCCTGACAAGATATGTTAACATAGATGAAGTTTGGTATT |
| | | ACTTATAGCCAGACTATTCTTCAACATATACACTGGGTTTTAGGAGTGTGCAATTT |
| | | ATAAGGACAGTTATATTCCTACAATCGTTGTATGATCCTTTTGGGTTTGGTAGAAC |
| | | TACGTTTGGGCCGCGCCTTTGGTCAACCACGGACTTTCTGTCTAGATGCCAATTCC |
| | | TACAAGCTTAGTCCTATCAATTTAGTAGAGAACAAATTTTGTCATCACTGAATTGT |
| | | CGTCTTACTATCGGATCATTCTCCGCTAATTATAGGATTATTAGTAACGCGTATAT |
| | | AGGAGCGATTAATGACTCATCAATGAATAGCATCACTAGGTGTATTATATGAACCT |
| | | CTCTCTATTCTATTAACTGCCCACTGTGGGTAATTTGAGTTATACCTGACCGGTCC |
| | | CTCGGATCCTTAATCCTTTGATGTCGATAGGTAACTGAAGTGTAAGATCCTGATAT |
| | | ATGAAGCCGGTAAGGAGACGGAGATTTTATATTAGTGTTCTTGGATACTGTGCTAG |
| | | AAGGTTCTACTCTAACTCAAACAGGTTATAAAGTAGGAAGGAAAAAGTTGATAGTG |
| | | GTAAACTAATTATGAGTTGGCTTGCTTATTCCAAGTTAGCGAGGTTTTCATGACGT |
| | | AAGTCTGATAAGGTTTGCTGGAAGCTGAAAAGTTTTACAAAAACGTTGTTTTAGAA |
| | | TGGTTTGTCCCCGAAAATCGAACCTGGCATAGCCCTCAGGAGACGAACAAGCCCAG |
| | | GCAAACCGGGGGTTTCTCGCTTATTGCTATAATCACCTCTAGTGTTGTAGAAGCAA |
| | | TTACGGTGGGGAGGCGTCAATGTGGCCTGAGTTCCGTTGAGGACTTTTCACGTGTA |
| | | GGACCCATTAATAGAGGAGATATATGTCTTTCAGCTGCGGAATTCATAATAGTGGA |
| | | AAGAAGAAAAGGGATTACTAGATTAATATTACTCATCCCAGACTTAAGTTGAAAGC |
| | | TACATCTTCACACCCAGGAAACCGGACCGCCTTTGTTCAGGTCTAAGTAGTCTGGA |
| | | ACAGAACCGTATCAACTGCCCCAATTCATAGGTGTTAGCGTGACAGCGATCGCGGA |
| | | TTTTTAGTCCAGACTGGCTGGGCCATCCGCTTCAATAAGTTAGAGGACTACATACA |
| | | ACGATGGACCCAATTGGCAATAGTCGTGGTAAACTTCGAAGGGGCGGTGTAAGATT |
| | | CAAGCTGTAGTCGTGATGAAGGAGATCATCGTATAAACAG |
| 7 | 39.70% | ATACATCTAGACTACTAAGAGGGATTATCCCAGCGCAGTCCCACCCAAACATCAAT |
| | | CTGTCCCTTTGTTCTAATATATCTCTGGTCGCGAATGAGTAAACGGGGCTAAAGGT |
| | | CCATTATTTTTATGTAGGAGCATGTTGCTTATTATGGCATAGCAGTCGCCATCCCC |
| | | CTGTCACTCGATCTAGATACATCTCACATTGATTGGAAACTTCTACAAAACGTTAG |
| | | TACTTAAGATGAGTGATTTAGTGCATTTCTCGTTTTCACAAACTTTGCTAAACAAA |
| | | CGTATTGAGTGGCGCGTTTTTTGATTTGTCGCATAACCGTTTACTCCCTGTTCGAA |
| | | GGAAATCGATCTCCTTATAAATAATGAGTACATTATACAGCTAGCATAATCTGCGT |
| | | GTGGCAAAAGTGAACGTTTAATCTACAATTGATGGAAAAATAGCCCGTTAGTCCTT |
| | | TTAAAGACGTCTTGGAAAAATATTGAGACAACCTTCGTCCAAAATATGTCAAAGCT |
| | | TCGTCACATCTTTTCACCTATTACTAACTCCGTAGTTCAACTGACTTTAGAGGGCA |
| | | AGTTTTGAGACAATATCTTAGGGCTGACTAATAAGACGGTTATATTTCAAGAAGGA |
| | | AAGATCTTAAGAGTCAAAAAAACGTCAGGGCTATCGTTACGATATTGGTATGAACA |
| | | GTAATGATATATTTTGCAGATCTTAATATAACGACATTCGAACACAATAGCGTCAG |
| | | ACAAAGGTTACCACTCCTCTATAATTACTGCAGCTTCAATTGATGAGCGTCATTTA |
| | | ATTTTGGCCGGACATTTACATCGTGAGCTGGCAGCACGCTCAGCTTTATTGTTCTT |
| | | GCCAGAACATTACGAATAGCCGTTCAATGCCAATTAGTATGATAAAAGTAGTGAGT |
| | | GTAAAACATGGCCTGGGTTTAAAGAATGAGTAACTATTATTTTGTAGGAATAACTG |
| | | ATTCCCTTGAGTTCTATCTTAAGTTGTACAGAATCACACTCCTACAGCGAATAAGC |
| | | AACGACATAGAATCCGTTATTTCGTATGTCTCGGCGGGACATGTATAAGTAGCATA |
| | | CGTTATATCGGTTGTCGCACGAACCGCCTTCATTCCAAAGGCGCTTACAAATCTGC |
| | | AGTAAAAAGCTTAGCATTTACTATAGAGTATCGGCGTTGACCGTTAAGCCCGTCCC |
| | | GTCCATTCAATCACTCAATTGATCATCTTTTGGCAATAGTCGTCATATGAGAAAAT |
| | | AGCTCTGTCGTTGTTATTATTGGCTAGAGTATAAGCTGTTAAACTACAGAATGACG |
| | | TTTTGTGGAAAGTGGACGTAAGATCCTTGTTCGCGAAGACTCGCACGGTGGGGAAC |
| | | AATTCCTGGGAATATTTGATCTACGTACGGTTATTCTGCATGTGATTACAATATTT |
| | | CCAACGCAGTCCTTTTGACATTATATGAAACCAGACCCGATGCATATGTTTTCTGA |
| | | CTGGTGGTTTGAGTCAGAGTCAACAAAAGTATCAGTCTTTCGTTACTAAATCTTCC |
| | | TAAGTAAATGGTGGGCGACCATTCCTTGTAACCTGTTCTGTTATAGGTACTATTCC |
| | | AGCCTGGAAATCGTGGAACACATCGATCTAGTTGTCTATCTATAAGAGAACACTCG |
| | | GTTCCAAATATGTAATCCGCACGTAAGAGAGGAGTCTCGTACATGATATATAACGT |
| | | TGGGTACATTTCTTAGACATTCCGGTGATACATAATGTACAAGTCACATGATTACA |
| | | CCAGCTGGTAGATAGAATACCTGAGACTGGGTCCTAGATGATTATAACAAGTGTTA |
| | | CATGGACGCTCTCGTTTTGTTGTTGGCTTAACACCAGGGCTTGCTCCATGTTCTCA |
| | | TGTCGTTATTACTGAATTATCTTCCATTATGATCCTGGACGGATGAACGAAGCAGA |
| | | AGATAACAAAGATGACTGAATGCCGGAAAAGGAATTAGGCCCTGATATATCGCGCT |
| | | TCTTTATGCATGTTTACGCTGTACCAATAAACGCAAGAGG |
| 8 | 40.80% | GTACCCGTATATCGTCACTTCATTTGAAGCTATTATTAATGTAAAATCCTTCCGTC |
| | | ACACACTCTTTTCAAAAAGGGAAGTCTAAATTAACATTCAGATGAAAAGCGCTGAC |
| | | CCACATGGGAATATCCTTTCTACGCTATCAGCCGAAAAGCTCCAGCGATTAGCTAA |
| | | ATATCTAAGCCTCCAGAACAGAGTTATTATATATTGGTTCGAATATGCTAATATTA |
| | | CAGTAGAAAGTAAGGTACCGGCACTTTTAACGCCGAAGTCGACCGGTGTAGCTGTG |
| | | AAAATATATTTAGTACACGTAATATTAATTGGAAATTGATGAGATCGAATCTTCAG |
| | | GAGAATCTGACGAGCATTACTAATCGCGCGTGACGGGAACGTTAATATACAAGCGT |
| | | CTATTCTAGGTTATAATAAACTCCTATCTGGCAAGTTGAATGGTTTTTTCAAAACT |
| | | TTAACGTTCTGGCTATACAAAGCTAGTTGCTTTAACTTATCGCATACTATGATCCT |
| | | TCCCATCAATCAATCTCAGTGACTATAAACGCAAGTGACACAATTGTCTGCGTTCC |
| | | ACATTTCTAAATCTCTTATCGCTCATTCCCTCTACACAAAGTTCGATTACCAAACG |
| | | CGGGTCTACACACAAGCTTACAAGGATTACAATATCCAATTTTTTGTTATCAAAGG |
| | | CGAACTCAACGAATTTAATCGTTGGTCATTGGTATGGAATGGCGATTATAAGAAAA |
| | | CTCTTTTAGTCATAGTAGCTCGAGATGAAGTGAACCGGGCCAGTCGGTAGTTTCAC |
| | | TATCGCGCAGTAGTCACGATCAGTTCTTAGAATCTATCTCCTAATCAAGTCCAACA |
| | | AGCAATCCGAAATGTTGCTTTCTATAAAGGGTATGTGTACCTGCCAATATTAAACT |
| | | TGATTCACTCAATAGTGATTTTAAATATGTCCATATTTATGCAAGAATCATTGACA |
| | | TTAGTAAATTCAGCCGTGCATTTGACACAATAAAGGTAGATTTAGACTGCATATTT |
| | | CCCGCATATTTATTATTGTCAACGCACAAAGTTGATGGACCGACCACGATCGCATC |
| | | GAAGACCGTCTAAACGACGATATTCTTCGGAGATCCATATTTGTTTTCAATTACCG |
| | | ACCATTGTTCATCAAGTGTAGTTCAGTCGGAAATTTTTCGTGTGCTTTTTAAAATA |
| | | CCAAATCTGAGGAAAAAGCTCGCTAGATGTTGAGTCAATCCGTAAGAATATGCCCC |
| | | AGGAGACATATGTAAGTCACAGCCGTAGACTCTCGGTTACCCCACGATATGTTCCA |
| | | TATGCAACGTTTGTTGAGTAATATGCAGTTCAGTCGGGCGTATTATCAACAGACAG |
| | | ACTGGCACAGTAAATTTTATCATCGGGTTTAAAATATCTAGATACCTCAGTTTCAA |
| | | GGGGGAGTTGAACTTTAACACGAGATCAAACTACATACACAAGATTATCAGTGGGT |
| | | ACGCTGAGACTTATCCTTAGCCTGGAGAGAGTCCAGCTACAGGAACTGCTAGTACT |
| | | TAGCGTGCGACCTCAAATCGAGAGAACTAATTACCCTGATCGACAGATCGGGCAAG |
| | | TTAAGCAAACGCGGCTCGCGTGTAGAACCATAACAATTGGAGATGCTCCTGCTTAA |
| | | GAGATTATAGAACCGCAACCCATCAATCGTCAGTTACCCGAGGGCTCACGCACGCG |
| | | GTGATGGAAGTTAGTTCCTTTGTACGCACGAGCTGCAATACGTGGTGATTATAATC |
| | | GGCGCACACTAAAGGGGTGGATACAATAGTAGAAGCATATACGTCGCATAGGCGTA |
| | | CGCGGGCGAAAATTTTAATCGTTAACGTGGCACTAACAGCGTTTTGTCTCCCCACT |
| | | CGTGGGTTGCGGTGCATCGCACATATTCCCACAACACCTCTTAATGCTTTATTATT |
| | | TGTATTAATGGCGCGAATCTGCCTGATATTAGTATTCGCACTAGTGGGTAACGAAA |
| | | TCTTAGTCGCTGGCTACTGCAGAACTAATTGCGTTGCGAT |
| 9 | 40.80% | ACTAGCTACAGATCTGTAATAGAAAAATGCAGATGCTTGTTCTGCGTCGACTCGCT |
| | | CATCAACATCCTGTCTCACAAGTTATGCATCCTGTGCATTTTATTGAAGCTTTGAT |
| | | GGGGATTAGATCGTGTATGGAAATGTTTATTCGCCTGGATAAGATCTGTCGGCTTA |
| | | TTCGTGGCCAATAATAGGTCAATTTGCGGAAACATAAAGACTCGCATACCAATACT |
| | | CGCTTATCCTGAGGTTAAATTTAGTGTATGTAGACGAACAACAGTATTTAGTAGTA |
| | | TGACGTTCCCCCGTATTGCCAGAACTCCTGAATATTTGGATATGAGGTATGACTAC |
| | | GAAAAAAATACTACGTTGCTCATAACCATTGGTGCAGGGATACCGAACTCATTGTT |
| | | AAGGGACGCCACAGTCCAGTCTCTTTTCGTTCAGAGCGTGTTTTTCAAAGTGCTTG |
| | | TATTAGTGTGGACAGAGTTTACTGATCTCTCCGCACTTGGACTGATTGTGATCCCG |
| | | ATCATCTCTTTTCATAATTGTAACACGCTTTCATAGTACACTTCTGTACATTGAAG |
| | | AGTGCTTGCAGCCGGACAGTCCTATAGAATTTGGCGTTTGTTCGGCCAATGTGTGC |
| | | ATTTTAACTTTAGGCGCCATCTCTTGAGATTACTCCTTTGAAAAATTTTGGCGGAG |
| | | GTTAACTCTGGTCTTTAACATAGGCGTGCTTAACACGAGCTTTACGGTCAGGTACA |
| | | GGTAACAAAACAGGTCTAAATTTATTTAAGCAGCTTCTGATACTTTCCAAGGGTCA |
| | | CAGTTGGGGAGCCTTCCGAGGTATGACAATCAGTTTTCAAAAGGTGTAGAATATCA |
| | | TATATTCTATCTAGGCCAGAGCATTCTAAGCTGTTAAAAGAGTGCTATGCTCAGAA |
| | | GTTGACTGTTCTAATCGAAAATCGGACATAGATAACCCGCATACCACAAGTCCCGT |
| | | TGTAACGTACCCATCGTTTTTGATTCTATGTCTTTGCTAATGATTGGCGATTGAGA |
| | | CATCCTACTTCTGTAGCTTGGCTGTTATGCGATCCAAAATGGTATCCAGTGGTGGA |
| | | TGTCCGCCGCAAACTGAAACTCCCTATCAGTTCTTTGAAATTAATTTGCGGGCTAT |
| | | CCGACTCATTCTTTAGGAATTAACAGAAGAACACGCGTCTGTACCAAGGTTCTTCT |
| | | TTGTTATATCACATAACAATGAATCACGTTCTATGATGAATCCAGGTATAGAAGTT |
| | | GTAGGTAAGCACTTGTATAAGGGGGCGCTCCTCTCAGATTGATTCATTATTTACTA |
| | | AAAAAGGAGCGTGTTATTACTTCTAACAACTCCTCGCCATTATATATTATTTAACT |
| | | ACCATTCCCACTAGAAATGGATATCGTGTTCTAAGACCCTAATTGTGCTCATTAAA |
| | | CTAACTACCGCACCAACCGCCTTGAATCACCGGACCACACTAGTTAAGCTGCCGAT |
| | | ACCCAATATGGTATTTTAGTGTATACCGGATATGACCTTATTTACGAATGGATTGA |
| | | GCTCACCCCATAGATCAGTACCAGCGTTATTATGAAAATCTTGTTATTTTAACAGA |
| | | GAGACATGCTTGGTCATTACTACGAATTTGAGTTTACGTTATACAAGGCGATCCAA |
| | | ACGGACAATAGCGCGATACGAGATTATAGTACCAATAGCACGAATCAGTTTTAGCG |
| | | ATCTCGTCCGATCTGTCAAGCCGAATGACTCTGAAACGTTAGTATCTGAAACGTTT |
| | | CATTCAGCCTAAGATATGTATAGTATCATTATACCGTGTGGGTAGAACAATCAAAT |
| | | GCAGATAAAGCTATTTAATGCACTTCACATAACCTCTCCGTTGGAAATCCATGTAT |
| | | TCTCTAATCAATTGAATTGTACCTTAGAAAGCACAGGGGGACACCTGAAGACCTCC |
| | | CATCTCTTAAGGTTACCGGCACGTGAAACTTCAAAAGTCAGACAATCAAACGGCAA |
| | | CGTGAATGTCTTCGGAAGTGGTGGTATGCACATCGCGTCA |
| 10 | 41.70% | TTAATAGAAGTAATAAGTGCTATTGGACTAAAATCGCGTCAATTAGCTATAGAACA |
| | | GCTCTGTGACGAACTATCAATGGGGCATTCGTTCACTAGTGGATACCGTACAAGCT |
| | | CGCCGTGATCGTGCGTCAAGGATAGTGCCAGAGCGCCGCGCTATATGTGTAACGAC |
| | | GCATAAGTAGATGTTTATGTTATTGGGCAAAGTCATTCTTATCCATAATAAGCGCT |
| | | GCCGATAAAGATTCATCAGAGATATTGAGATTCTCCATACTTGACTAATCTCTGAG |
| | | TAATTAAAATATATTTCTAATCGGATAAGTTAGGGATCACCGAACCCAATGAACTT |
| | | AGTTTAATGTGTTCTCGCGAATATCCCCATGATATAAAGATCCGAATACCTCAGCT |
| | | CCGTGCGTGCTCGTGCAGTCGTGCGTTTTCTATGAATCAACCATCAGTAACGAGTA |
| | | GCGGTAACTACTTCTCGAGTTTAACCAAAGCCTATGTATACTAGCGTGCAATCACG |
| | | TGCGGAAGGTCCGACCTACAGCAGCATTTTCGTTCGAAAAACGAAAACTAATGTGC |
| | | ACTATGTTGAATGGGCATTCAGGCCTTAACTTCTAACGTTAAACTAGATTTGCGAT |
| | | TATTAGGTATGAGATCGACCAGGTCGCCACAGATAATTAAAGATAGCCCTAGCAAA |
| | | GTGATAAGGTCCGGATGTTAGAACTTGCAAGAGTGTGTAAGATTATTTACTCTCGG |
| | | TGCGTCGACAGGCGAAACCCATAACTTTTATCGGTCAAGATTACGACCTTCAGCTA |
| | | GTATCTTGAGATTTGAAAGGGCCTAAAAGCAATTTAGTGTACTTGTGTAACATAAC |
| | | CTTAATTATTGATGGTTCTATCGACTCCCAGCGGTAATAATCTTGTAATATTGTCG |
| | | GATTTAGTTGAAGGGCAGGTTGACATACCGAACAATAGCTAGTATCAATGTATAAC |
| | | TAGCAGGCATCTAATTTCGTAAACACTCCTGACACTTGTCGTGTCTAAGCATGTTA |
| | | GGACAAAAGACCAGTTTTTTTAAACCTGACTGTACCGGCAACGCCACAGATTTTAT |
| | | GTCTCGCATACGTACGAACTGAATTTGAGGGGGCTCAGGTTTGGACTTACACCGCA |
| | | CGTGACTATACTGAGATCGAGGCTCCATTAACGGCAACATAAGACTAGCACTGTAT |
| | | GATCTGAAGCCAGGCTCTGGTGAAATTGCGGGTAGTTAACGACATTTATCGACGAA |
| | | CCCTTGATAAAAAGTGATTATGTTGTATCTGCGTGATATATTCTTTTCGTGTTCAG |
| | | TCTCTAGAACTTCGTGCGTAATAAAGATTATAGAGGAACGGTTAACCTCATTACAA |
| | | GACGGAGACCGTTCATAGACGCCGATGGATTACAGGGTCTACTATAGCTACCTAGA |
| | | ACACTGGTGAACATAGGGATAACATACAATTAACAATATTCCGAGCCAAATTATGT |
| | | CTTGAGTCTTGGTTGTTATCTATATCGTTATTATGTTAGAAACTAATAAATGCGAT |
| | | AAGAACTAGATTTTACAGTAGATCCAAATACCGGAATCTATCGGGACGATTGATTA |
| | | AGACTTACTCAAACCTAACTTTAGCCCGATTTTGCAATTAGAGATACGTCGATTTC |
| | | GAGACAAGAGTAGCGTCCCCATGGCAAATATCCACGGACAGATAATGACACGTGAG |
| | | GGATGGCAAGAGTAGTTGCTCAGGATGTAGGCGTTGATGGTCTGGCGCTAATGTCG |
| | | TGGCTACCTGTTGAGTCTCGCGTAATGACTAGTAGTGTTCGAACGTATGACCAAGT |
| | | TCCTTCCTAGTGTTACCACTTTGACACATACCCAGGGGTTTGCCGCATGTCGCTAC |
| | | TATAGTATAGGTGCTGCTATGAAGCTTCTGAATCAGCGGCTAACAAGTACCTAAGA |
| | | AAATTGGACATCTTTTGGATGACAGTGCACAGGAGCCTATACTGAATTATCGGTGA |
| | | TCGATGCTTCATGTAATCAAAACCAGCGCGTACACACTTT |
| 11 | 39.10% | TACTCTTAATTCATTACATATTGTGCGGTCGAATTCAGGGAGCCGATAATGCGGTT |
| | | ACAATAATTCCTATACTTAAATATACAAAGATTTAAAATTTCAAAAAATGGTTACC |
| | | AGCATCGTTAGTGCGTATACATCAAGAGGCACGTGCCCCGGAGACAGCAAGTAAGC |
| | | TCTTTAAACATGCTTTGACATACGATTTTTAATAAAACATGAGCATTTGAATAAAA |
| | | ACGACTTCCTCATACTGTAAACATCACGCATGCACATTAGACAATAATCCAGTAAC |
| | | GAAACGGCTTCAGTCGTAATCGCCCATATAGTTGGCTACAGAATGTTGGATAGAGA |
| | | ACTTAAGTACGCTAAGGCGGCGTATTTTCTTAATATTTAGGGGTATTGCCGCAGTC |
| | | ATTACAGATAACCGCCTATGCGGCCATGCCAGGATTATAGATAACTTTTTAACATT |
| | | AGCCGCAGAGGTGGGACTAGCACGTAATATCAGCACATAACGTGTCAGTCAGCATA |
| | | TTACGGAATAATCCTATCGTTATCAGATCTCCCCTGTCATATCACAACATGTTTCG |
| | | ATGTTCCAAAACCGGGAACATTTTGGATCGGTTAAATGATTGTACATCATTTGTTG |
| | | CAGACCTTAGGAACATCCATCATCCGCCGCCCTTCATCTCTCAAAGTTATCGCTTG |
| | | TAAATGTATCACAACTAGTATGGTGTAAAATATAGTACCCGATAGACTCGATTTAG |
| | | GCTGTGAGGTTAGTAACTCTAACTTGTGCTTTCGACACAGATCCTCGTTTCATGCA |
| | | AATTTAATTTTGCTGGCTAGATATATCAATCGTTCGATTATTCAGAGTTTTGGTGA |
| | | GGAGCCCCCTCAGATGGGAGCATTTTCACTACTTTAAAGAATAACGTATTTTTCGC |
| | | CCTGTCCCTTAGTGACTTAAAAAGAATGGGGGCTAGTGCTTAGAGCTGGTAGGGCT |
| | | TTTTGGTTCTATCTGTTAAGCGAATAAGCTGTCACCTAAGCAAATTAATGCTTTCA |
| | | TTGTACCCCGGAACTTTAAATCTATGAACAATCGCAACAAATTGTCCAAAGGCAAC |
| | | AATACGACACAGTTAGAGGCCATCGGCGCAGGTACACTCTATCCACGCCTATCAGA |
| | | ATGTCACCTGGTTAATGGTCAATTTAGGTGGCTGGAGGCACATGTGAAGCAATATG |
| | | GTCTAGGGAAAGATATCGGTTTACTTAGATTTTATAGTTCCGGATCCAACTTAAAT |
| | | AATATAGGTATTAAAGAGCAGTATCAAGAGGGTTTCTTCCCAAGGAATCTTGCGAT |
| | | TTTCATACACAGCTTTAACAAATTTCACTAGACGCACCTTCATTTTGTCGTCTCGT |
| | | TGTATATGAGTCCGGGGTAAGAATTTTTTACCGTATTTAACATGATCAACGGGTAC |
| | | TAAAGCAATGTCATTTCTAAACACAGTAGGTAAAGGACACGTCATCTTATTTTAAA |
| | | GAATGTCAGAAATCAGGGAGACTAGATCGATATTACGTGTTTTTTGAGTCAAAGAC |
| | | GGCCGTAAAATAATCAAGCAGTCTTTCTACCTGTACTTGTCGCTACCTAGAATCTT |
| | | TAATTTATCCATGTCAAGGAGGATGCCCATCTGAAACAATACCTGTTGCTAGATCG |
| | | TCTAACAACGGCATCTTGTCGTCCATGCGGGGTTGTTCTTGTACGTATCAGCGTCG |
| | | GTTATATGTAAAAATAATGTTTTACTACTATGCCATCTGTCCCGTATTCTTAAGCA |
| | | TGACTAATATTAAAAGCCGCCTATATATCGAGAACGACTACCATTGGAATTTAAAA |
| | | TTGCTTCCAAGCTATGATGATGTGACCTCTCACATTGTGGTAGTATAAACTATGGT |
| | | TAGCCACGACTCGTTCGGACAAGTAGTAATATCTGTTGGTAATAGTCGGGTTACCG |
| | | CGAAATATTTGAAATTGATATTAAGAAGCAATGATTTGTACATAAGTATACCTGTA |
| | | ATGAATTCCTGCGTTAGCAGCTTAGTATCCATTATTAGAG |
| 12 | 40.90% | GGCCCTATAGATTTTAACCTAAGCTCTAGCTTGTGTGTGCTCAGAGTACTGCTCAT |
| | | AAATATGCTCGATAAAGGAGGTAAGGCATATCGTAATTTGGAAGATAATACCACAC |
| | | TTATTGGTAACACGTTGGAATCACATATTAATTATGAGCCAGCCTTGGCATTCGAG |
| | | CAGGGATATGTGGGAGTATCAGTTGAGTTTGGCTCCTTGCTACTGCCCTCTGATGC |
| | | TCTGCTTGCTCTAGCTTAGGTCATTAATGATAAAAAAGAGCCAGAGTGTGGGCTAA |
| | | ACAGGCAACGGTACCGTTGTAGAGCGAGGTATTGCTATCGGGAGACGTCGGGTCAA |
| | | AGTGGGATTCATGCAGTAAGTTTGCCAAAGGGTCTGCTTAAAGAGACCGATTCCGG |
| | | AAGGCTATATGCCATAGCAAGGTATGCACTGCATTGAGCTGAAAACTCTTGAGCAT |
| | | AGTATTTACTAAATAAAGAATCTGATATCTTCTAGCGTGTTCACTGGACTATTATT |
| | | TAGATGGTCGCCAACAACAAGCGTGCGAATCATATAGACCCAACCCAGGGTGGTAT |
| | | TGAATTCTATATTAAAATGTCTCGCCCTTATAACTCTCTAGGTTTCCATAGTACAA |
| | | ACCTAGGTGTCGTCAACTGCATGCACTGCTTTTTGTATCGGTAATGTTGATCGACC |
| | | CGATGGGCTTTTTTTAATAAAGGTCTTGTTTAGTTGATCATACTACCAATTTTGGT |
| | | GGTCGATGGCTCAATGACCAATGGAATCTTTATAGTAAAAGAGCCCTTGGCACCAA |
| | | CGAATCATGGAATTTAGGACGATGTCTCATTTACCATATTTTGCATTCAGACTATG |
| | | ACTTTCAATAATAGAATATCATCGTCAAACACCGTGGATATGGCATCGACAAGTGT |
| | | TGGGATGCCCACTGAATAACGTCTCTTCGTCATCTTTAGGGCGGCTATCCATTAAG |
| | | GAGGATTTTATTTTTATAGCAGTCTTAGTCCGAGGCATTGGCGCCAAACATCGGCT |
| | | CAACACTAGACACGTCTTTAATGGAAAGTATCTAGTGTTACTGCGGTACGGAAAGC |
| | | AAGTTCAGTACTTTTATCCAATCTAAGTATCACCCAGCTTATATTTAAAAGCTAGG |
| | | TAATAGGGAAGTTACTAATAACTCATGCGCGTGTAGTGTAGTCTTGCTGTCGCTTA |
| | | AAGCAACTGAATGAATGTACGGCTGACAAAGGCTTACCCAAGAAAACTCTCTTGTA |
| | | CGCTACAAGAAACCTGTAACAAGAGAAAAATATTTTAGCCCACGTATAGTGAGGCC |
| | | AAACTTGATGCCCGTAAAAGCAAACAAGTAATATTCAGCAGAATTTGCGGTCATTC |
| | | AAGTGTTTAGGTACGTAACTTTTACAGAATTAGCTGTTGATTAGGTAATACTAAAT |
| | | CAAAATGTCGTAATACCGAAGCAGAAGTATATGATCTAATTTGTCGCCTCGCTTCA |
| | | TGCTACGAATGTTACTTCGTTTATTACAGCTGCAAACTTGCAGTGACTTGCATTTG |
| | | ATAGGATTCTTCCTAGGGAACCATACTGGGCCGCGGACAGGGAGTCAGGAACTCAT |
| | | AACGGATGAAGATGTAATCTCTATAGGGGTGAATAACAGGATTGAAGATAGTAATC |
| | | TAAGTACTCTCATCTCGTGGACGACTTTAAGCGCACTGACAGCGACTCGCGATTCG |
| | | ACGAACACCCGTGATCGATTTACACGTTCATTCTGAAAGATATACAGGTAATAATT |
| | | CTAAAAGATAATTGAGTACCAATATATAGGTTTTATGATCTTAGGCGCATGTCACT |
| | | GACGAGAGAAAAGATAGTCTTGCCGCCTCTAAGTGTTCTATTTCTGGACGTGCCTG |
| | | GGCATTAAGGGCGACGTTGACTTTTATACACATTTCATGTCCACTAACAATTTTAT |
| | | ATCACGTAGCAGGACATAAAGGGAGGACTCTATAAAAAGTTTCGCTATATACGTAC |
| | | AGTACGTTCAAAATCTCCAGAGGAAAGCTTGTAAAAAAAG |
| 13 | 40.40% | CGCTCGACACGAGTATAACAAATATCGATAGATGCTATAGTGATAAGGTATAAGTA |
| | | AAATAGTACTGCGAATACAAATAGCTTGGAGAAATACGTTCATCCTTTAACTTCAA |
| | | AAATTTTTGGACCTCAGGCACGTTGTCATTATTACTGGCAGGTGATACCACCCAAA |
| | | AATCGTACCCGCAATATATCTTCGGTAATTCTTGCCAAGTTGGGATTTTACATACT |
| | | TAGTATTAATAGTGGGATCAGCTTCGATCGAAGACCATAACTCAGTATGTGTATTC |
| | | CTCATACAAGATTTCTGAAGGACGAAGGCTCATCAATGCTGAGGTGTTATCAGGTC |
| | | AATAACAAGCCGCATTAACGCCGTAACCCTAATGCCATAATTCTTTGACGAAATGC |
| | | CAAATAGTTTCATCAGGAATCACATTATTTGGATAAGGAAGCACAACAAACGCTTT |
| | | AATCTATACCCCTAGAATTAAGAGGACAGCATGATAGGCTTTGCAATGAACCAGTC |
| | | TCCTAAGCGTACCACCACTCCGGAGCCTTATGGCGCGCCGGTATTATGGCGATGCA |
| | | CTGCCTGGGCGAAACTCGAGTGAATCATTTTTCCCGATATACACAGCAGTACGCCG |
| | | ACGGTCTGGTAAAAAAAACGTTATAGGCTTTGACCGCATGGTGATCGTGGTTAAGT |
| | | GCCTTTACCTAGAGTGCTGCTAGATGTAACACAATTGATCTGACAGTTTACGACCT |
| | | TGTAATCCAAGAACCATATAGATGACCCGCTGAGTTAGTAAGATAATGCACGCTCC |
| | | GGGGCTAAATCTAGTGCGGTTCATGAATACCGAATCAACTACGGTTATTGGCTGCG |
| | | GTAGAATATTTAGTTGTGTTAAATATACTCTAAGATGAACATGTATCACTATAATC |
| | | ACTCACCCCCTCTGCGTTCATAAGTAAGTGGCTAGTGTGATAGTAACTTGTATCAG |
| | | CGACCACTACTATATGTGGAAGCTTTTGAATGAGAATCTCCGCACATGATGATGTA |
| | | TTGATACAATTCTTTTGTTCGAAAAAGCTTCGGTGTTTTTTAGGACAGGAGATTAA |
| | | CGCTTTAGAGTCATACATATATGTCAAGAAACCGGGGAAAAAATGCCAGCCCAGAG |
| | | TGTTCTAAACGATAGGTTGTTCAGTTTTTAATAACCCGCGACGCGTCAAGTAACGT |
| | | CACGGGTCAGCTACGATTACCAATTTGCTATAAACTTTCCCCCGACGAGCCAAATC |
| | | CCTCAAAGCTGCCAGATAAAAGGATAGCAACCTGTACTCCCCGTCAAATCTAATGC |
| | | ATTCTTGTTTTTTAAGTCTCGTGTAACATGCGTTGGCTAATCTTCTCTACCGGGTC |
| | | CAGTGCCCTTTCAGCTTATGCCTCACCTTTGATTAGTAATGGACATCAGCTTTTAG |
| | | TCACATCGGAGTGCCAATTATACCGTTATATCTTTCTCTGATGCAGACCGACCTGT |
| | | CGTGTACCGATTCATCCTAGGGTAACTAGCCGTGGCAAAATATCTTTATCGTGTTG |
| | | TCAGGACTTGGTTGTTATATACTCTAGCCCGTAGATTTAAAATAAATTAAGTGTAG |
| | | ATCGTCCAAATATCTAAAGCAATCGCAGTTTTTATCACATCATGTGTTAAAATGCG |
| | | ATCAAAAGAAAAATACTGTTATTTCGAGAGTCAAGGCTGTGAGGAAATATGATGAA |
| | | GACTGCCATCCTGGTGGACTGGCGGCCCCAACGTTGAAGTTTCTATTTGATCGGTT |
| | | ATTAAAGGATACTCGAGAACAACATCGAAGGAATAAACTTTTATAGAAAGTCTCCG |
| | | AAATGAATAACTTAAGATATAAATTTATCGCGCGATAGTTCTGGTGGATGATAGCT |
| | | TTATTCCTCTTAATGCAGTATAGCTATTGCACCTATTAATTTGTATAATAACGTAT |
| | | CATGTTAGACGGTCAGCATGATATTCCGGATAGTGGAAGCAAATTACGACATCTAA |
| | | ATATGTCGCTAGTATTTGAGTCATTATAGCTTCGAGGCTT |
| 14 | 42.10% | CTCTAACGTGCATTTCTTCGTCGCCTTTGTAAGACCCCACAAAAACATGACGCTTT |
| | | AGGGATATGGTCCAAGACTCCGAATTGAAAGTATGCTGGTATGATATGGGACGTTT |
| | | TTGAAACCCCCCTCTCACGCGGGTAATTGGGTTTTTAGTTAGTGTATCATAGTAGG |
| | | TATATCTACGAACTACGTCTGACTGAGAGAGACTTTGTGCCTCTCAACCGCTATGG |
| | | TGTCAGCGACTGATATTGGAGTTATTTACCCGTCGTTATACGTGGGTAATCTTTAC |
| | | TACGGTTCAAGGTAACTAATCTAGTGTAGGTAGAATGCTGAAGAATTACCCGTTGG |
| | | ACCCGGTAGTCCGTCCGCTCCACGCATGGAATGCATGAGTAACGTCTAGGTGAATA |
| | | TCCGGAGTGCATAACTTTTTGGTATCTAGTCCGCTACTGGATGCAGAATGACATAT |
| | | TTTTTTCGAGTGCTTACTATTACTCTTCTCAAACAGAACGATCATTATGTTGCTTA |
| | | AATTCACGCTATGTTCTCGATGTAAAACAATTTTCGTAGAGAAAGATGCGTAAAAC |
| | | GCAGAGTTAGCATATAAAAAGTACAATCAAGCCCGAAGCACTCACAAGAAACATAG |
| | | GGGCTAAATGTTACCGTCCAAGTGAGTAGGATTTAATATCAAGCCGGGCTTATTGG |
| | | GTACAGTACGTGGACGGACTACGACGCATGTGTGTTATAGAATGAAGTGCCTACAA |
| | | CTGAAGCACAATTACTAAAGGAATGTACCTGGGTTTACACTAAGCATCCCATCCTC |
| | | TTCGCGGTTCAGCCTGATGTAAACGTAAATCTCGTCTTCCCATTATTAAGACGCCT |
| | | CGATCTACGATAGGTGATACGTGTACATCGGTGGACCATGTGTTTTGATATTCAAC |
| | | GATGTAAGTATGGTTCCCTGCAGTGAACCCCTCTTCAAGTCGTCGATGTACCTGCA |
| | | AGTGTACAATCGGAAGACCATGGGTCCATATGTAAAAATAAGTTAGGGGTCTTTTG |
| | | GTCTGTGTTGGTTATAATCGATATTGCCAAAATATTATGGACAGTTAGTTCGAATT |
| | | TTGTGTATGGTAGCCGTCGAAAAGGGTGGACGTTAAGTATATCCATCCCAGCGGCT |
| | | GGGAGATATGTAGACCGACGAGTGTTAAGTTATTCCACTTACTTTAGGACGAAATC |
| | | AATACGATTATTTTACATCGGAGGACATGACAACAAAAAACTACTCGGTTTCGACA |
| | | GGTGGAAGATGTCGCTGCGCACCAGTAGAGCTTAGGAGAGCGACGGTACTCATTTG |
| | | CAGCATGGGTACGTAATCACGTTAGTAAATAAGTAAGTATGCCTTCTCTTATGTCA |
| | | TTTTATAAGCTATAATGGTGTTGTGCCAACTTAAAGATTGACACATGATATGCTAC |
| | | CAGATAAGCCTCGAGTCGCCTATATTTTGCTACTAAACCTGATTAACTAGAGAATA |
| | | GGTATAATCCCTGGTAACCAGTAATTTTAATACTATGTTGCCACTTGATGTAGACC |
| | | TGGCTGTGGTTACTAAGGTGCTTTGAAACCATTGACCACCCGTTTCTGCTCGGGTT |
| | | GTGCATCTAACGTAAATATTCAGAGATAACGTGGCTCTGCTATTATTTTTATATTG |
| | | CCTGCTGACATATCATCATCCTTGAATGGCCAGCAACAGTTCTTGATCGGCAGAGG |
| | | CCCCATGAACTAGGGTAATATAGCAGATTAACTATCGGTTAACTGTATTAAACTTG |
| | | TGTAATACTTATATTGACTAATTGGGATTGCCTTTGTCGTTATCTCGTTTATCTTG |
| | | AAAACGGTGATGTTTTTAGAGGCGATAGTATTGAATAGCTCGAATGATCACCAGCC |
| | | ATCAAGAATGTAGCTAACTCCGAAACTCCTTGACGAGAGCTCAAGCGAATACTAGG |
| | | TCGGCGCTGCTATCCGCAGAGTTCAGGGTTCTACCCGGGGTATAAAATCCCATTGA |
| | | TCATTCAGATATTATGGACTTGGCGTTTATGCGACGAGTC |
| 15 | 39.60% | AAGAAGCAGCTAGTGCTACTTCGGAATAGTTGTCGTTTAAGTCCGTTCAAACATGA |
| | | CGCTCTAGTCATTTTGAAACCTAAACCAGTAATAATAGACTGACTCAGAATGATTA |
| | | TACTGCTATCTCTAGTTTAAGGAGATCCAGCGAAATAACTTGGTGAACTATGCCGA |
| | | GATACTATAAAAAGATCAAGGACGGGTCGCTCACGGTTTTGGTTTATTTTACTACT |
| | | TCTTCGTGGCTGTATTAGTCGATGCAAGTTCTAATAAATAGCAAACGTTTTAAGTG |
| | | GGATTAGTACATATTGATGGACGTCCACCACGTCAAATCTCGCAGCGTCATAGAAG |
| | | GAGCTATAACCATTCACTGCGACTACGACATGTGTTTGGGTAGTGCCAACTACCCG |
| | | CTTCCGCGTCCCTGCCGTTCTGTACACTTATAAAATTGATATTTTAATCAGTGGAT |
| | | GTGCTGATACGGGGCACTGAGATGATGAATAGTATTAGGCTGTAGTACCTTATGTA |
| | | CGCAAGAAATTTTAGAGTAAAGATTAGTCTGTGGGTAAGGAAAAAGCTAAGTTATG |
| | | ATTATCCATGGCCATGGCATCTACAAGCTGATGAACGTACCAACATTATCTAATTT |
| | | AAGAACTTAACTTGTCTTATCCTCTCTTAAAGTCTTAATTTGCACTATTAAGCTTA |
| | | GGGAAGTCGCAACCAAACTCGTGTAGTATTGAGATAAATTATTAAACTTTCTTAGT |
| | | ATCTACTGATATCCGTATCAAGTATGCTTATAAATTCTTGTTCTGCCTGACAGGCT |
| | | AGTGAATCCTGCACCCGGGACGATTGCAGGTGTATACAGGCCOTCACGCTAGCAAT |
| | | CAATACCAATACGAAATAAGGGCTAACATTTTTCGTAACAGATTAGAAGCAGTCCC |
| | | GTTCAGAACTTACCACTGCACCAACGGAGGTACTGAATTCGGACTCATAGAATCCT |
| | | CGAGTAGTAAGACCGTAGAAGAGACAGTGCATATTAATGTCATAGATCAATTTATA |
| | | TTTTATATGGTTGCCCATTTCATGATACCCCTTTAAATTTATAACTTAGAAAAGGA |
| | | GCCGCACTAATAATGAGCGGCATGCTGTAAAAAAGTAGGCCAAAACGCAAGATAAG |
| | | GTACCTTTGTTGTCCAATCAAATTAATTGATTTATTCTTCGATCGATCGACCGTCA |
| | | TAGTTGAAGTAACTATTTAGTTACGGCAGATACAGCGTATCAATTCATTCGGTGAC |
| | | TTTGCTTAGATAACTGCTCGATAATCCGGAATTATCATCGTTCAAAGTCCTTCCCT |
| | | TACTAAGGCTCTTGGATTCAGATGATCGGTCATCCCTAACAAACAGCCCACTGCCA |
| | | TGCTGCTATGGTGACATTCGTTACTACATTGATTTCTGCAGACCTTCATCCATAAT |
| | | ACGATGGTAACGTCTCGCTTACTATGCACGGTGTGCCCCTGCCTATATCTTCACGA |
| | | TATACCAAGTGGAGAACCGTAGGCATGTAGTCATTCAGGTGGCCACTCTCCTTCAC |
| | | ATTATGTTTAGAGGTCATGAATAACCCTAATCGTGTGACCTCAAACAGCATCGTAT |
| | | TCCGAATAAGTAACAAGTAGGGGTGTTTCAAGTTGCATGACACAATAGGATATGAT |
| | | TCTCAACCAAACTTGGCAATAAACGCATAGGTTTAGCAGTACTAACAAGCCATTAT |
| | | GTTTAATATAGAGCATGGCTTACTCTGTCATGTTCAAGGTGGCTAAACCCAACGCG |
| | | TTAATACACTCATCGGTTACAGTGTTTTTAGAAGAGCAATTGATATCTCTTCAGGT |
| | | GATACCTGGTTCATTATCCTAATTCAGTTGGTTCAGGAAGCCTTATAACTACCAAT |
| | | TCGATATTTTTAAGCATATAGATTAGGTGATACCACACCGTAGGAAATTGTGCAGA |
| | | ATTTGGTGTCTAGAAATTTAACATTAAGTGATCAGAAAATTCTCTGTGTTAAACGA |
| | | CTGTTGCGAATCTGTGTCTTTCAACCTCAAGTACGATCTC |
| 16 | 40.10% | TAACAACCTGTAACTGTCAACTAATGACCTCCTTACCAAAATTGAGGGTAGTTGGT |
| | | TCAAAGAGAATGCAGCATGACGCAGAGCTTGTAGTCACATCGTTCTTCTAGTACGC |
| | | AGAGTGTAGAGTTAAGATTATTAAACTCAGAGCACGTTGTGGACAAACCAATACCA |
| | | GTCCATTCAATTACATGGTATCTAACAGTATCGTACAACTTTAATATGGTCTAGGG |
| | | CTAGTGAAGTGTACCAACTACTTGATACGCAGTAAATAATTTCATCCTATCTTTAC |
| | | GTCGCCATCGAAAAGCAAAGTTATGGCGCGTGGAAATTCAGATGAACCATAACCAA |
| | | ACAGATAAATTGGCAGCAGTTTTTTGTAGACATTTATATAAGAAGAGCTCGAGGCG |
| | | TAGGTTAATTCTATACAACGCTATGATAGTCAAGTTCTACTTGACCAACTACGCTG |
| | | GGAATGTTTATTAAATTCAACTGGGGGCAAACTAGCATATACTGTCTGAGTGTCCT |
| | | TCGATGGTTCTATACAAACGGGGTGTCGAGGTACTAGTGGAATGGAGAAACTACCG |
| | | ACAAACGCATATCTTATCTTCTACTCGGGATTTATGAAATTTTTTGCGTATACTAT |
| | | TCCTGTGAGCAATGTTCAACAGCGTAGTGAGCCTCATAACGTCACATCAATTGTTT |
| | | CACGTCTGTGGCTATCGAGTATTCCTTAACTTAACTAGAGTATAGACATTAGAGTC |
| | | TAATTCTATGCAAGTTAGATAACTACTACTACTGTCGTACTTCATTCAGTTCCTGC |
| | | TCGTACTCGGCGACGCTATAACCGGCCTAGTTTGTGCGTCGCCAGATAACTGTTCC |
| | | TTTTAAACGTATAAAAAGTACGAAAGATTAACCCAGCGGAAGTTGGGCCCCATAAA |
| | | TGTCATATAGGGACTCAGACTACTGTTAAAAACTCCTAGTATACATTGTAGATAAT |
| | | CAACTAAAGTTGGACTATCAAGAATCAAACTGTAATCAGGTCACAGAACAAATGGA |
| | | CTAATAGAGCTATCTAATCATCATACAGATTTATACCCAGTGGAAACAAAACTTTA |
| | | CCCCTTGAGGATTTACTGGAGTTGTGTCAAGTTAGAAATCGGTCAACATAAATTAG |
| | | AAAATGCCTTGGAACGCTGTATAACTGATCACATATAGCTGTGCCTAATGCTTCAA |
| | | TCGTCAATGCTGACCACAATCTACCTGACTTGGAAATCCGCTACACCCATATCCAT |
| | | ATACTTAAAGAATCCGTACTTTATATCCTATTCACCGATGTCCGATGTGGCGCTAT |
| | | GTGTGTCTAGTAGTATATCAGTTCAAGGCGAGAATGAAGAAGAATACAGGGTCTCT |
| | | TTAGAGCACTGTGTCACTGTTTCTTAGGCCAGTTAATTCTAGAAATCAAATAAATG |
| | | AATAACTCGCGACGGCTCAAAAGAAATCTATGGTTTACGCATAAGCTGTAGGTACT |
| | | TCTAAGCTTGATTTGCTTCCGGGGGATCCTAATCTAAATGTGAAGGGGCAGATTTA |
| | | GATCTCTGCTCATTGAGTGGGAGGTTGGACATTGAACATAGAACTACCTTCCCTGC |
| | | GTGCTGTAAGATTATGAGAATCTATGCTCGGTCGTTGTCTAAAAATCAGACTACAA |
| | | GGGTAAGAATAATAACAGACCGAAATAGATGTCTCCTTCAAGATAGTCAGTTTGCG |
| | | CAAGTCTGGCAGGAACGTTAAGTAATCCTGAGTTATAATAGCGCCCTTTTAAGCTT |
| | | TCCTGGCGAAAACCGAACCAAGCCCCCGTAACACAATGTCACTATCCGTACGAAAG |
| | | TTAGTGTAATAACGACTGTACCTATTATAAGCACATTTGGTTGGCTATCTTCTCCC |
| | | TAGATTCCTGGCGGAAAAGAAGCATGTCTACGTTCGATAGGACTCATTTTTGAGGA |
| | | AAACTATTATAACGGCTATAACGCGCGATTAATCCCTGTCGGTCCATCATTCACGT |
| | | GAGTGTAAAATTGTGATTAGTACTTAAACGGGTTCGTGGA |
| 17 | 40.70% | TTGACGATTTATATAGCTACTACTTAGCCTTACTACATATTCCGGCGTGCCGGTAG |
| | | ATATGACTAAGTTAATACTTACAGACATTCAATATTAGGATTTCGGTGACCTCGAT |
| | | CTCTCTTGATTGAATAAAAAATGGATATTAATGCGTCGATAGTTGTGATAAGTTAT |
| | | GTATGATGTCCTGAGGGACATATGATAATCTTCTAATAGTTACCTTAAACCGAATT |
| | | GTGTTTATGATGAAAAATATAGGTGAAGTTAGCACCTATCACCAGACTTTGGGATA |
| | | GTTAGTCCGTACCAAGCAGCAGTTCAACTGACAGGAACGTCAATTCTGTCTCTCAT |
| | | TACTTTGGCCATGGATTGAAAATCGACTTCAGTCTGACTCACAACAGTTATAGAAG |
| | | GATTTTGGCTCACCACTCTTCGAAATAGGTCATTTAATGCGTACTGCTTTTTTTGA |
| | | CGGCCCTTTATTCATTCTATTGAGGGAATCCCTAACTTTAGCCACACGCAAACTGG |
| | | TTTATATGGATACTCTCAAGATTGTTTACATATCCAGAAGCTTATACTTCCTCAAT |
| | | GTGATGCACACAAGGTGGGATCATCTTGTTTCTACAATGCAGAATGAATTAAAAAT |
| | | CGCCCTTCCTGGCACATCTTGCTGTACGGCTACAGAGTAAAATTAGCTCGTTATTT |
| | | ATGAGTGTTTACACAACCCAAATCTAAGTCGAATGTACTTTAAACTTGGCGTGGAT |
| | | TCATAGACATGCAATCAGTGTTAAATTGTCACTCAAACACGTGCCTGACTTCAGAC |
| | | AAATTCATGGATTCAAGCTGCTAATATTCACAATAGACGAGATAGGGGCGTAGCTT |
| | | TTTCTGTACGATGGGGGAATATACGAGCATTTCTATGAACCAAAACAGGCAAAATG |
| | | AGCAAATACCTTGTGCATCATATAGTTTCCATCAACTGGAGAAAGCCTCTTGATCG |
| | | GCTACAACTTTTCAAGTCCTTGCGGCGTTGGCCCTGAAGTACTATAGCCTTTTGTT |
| | | CTCACTAATCTAGCCAATCACTTGTTGACTATTCTTGCCTCACCCATAGAGTGGTA |
| | | ATGGAATTCCAAAAACCTATTCCCGAGTTTAACCCGTATTGTTTGAGAGGAGTTCC |
| | | TAGTGTCTTCATTAAATTGCACATGGACTCTACGGAAATTACTTTTTATTAAATCA |
| | | TAGAATCTCTGTCATCAGTCCATGCGTCCTCAGTCAATAACGGTCGCCGTGTCTAC |
| | | GGAAAGGTTCATTCTATGCCTGTAAAGTACATCTAACACAATTTAGTGTGGGTCTT |
| | | CTACTACAGTTCACCCGGGAAACGTTTTATGTACGAGTGTTGGTAAAGCGTCCTCA |
| | | TCAAGTCGATCCATTGTAAGGAATCGACTATATACTCCAGCTTAACTAGGACCCCG |
| | | TTACATCTTAATGGTAGGTCTAAGAGGTGATAAGACTGGAACCTACATCATGAGTT |
| | | GAGTGAGCAATGAGAGCCAGCAAATGGTGGGAAGACTAGACCAACACAGGATCTCA |
| | | TGCTTCCTGTAGCAGTGCAACTCAGTTCGCTGCGAAAATAATTAACATATCCCCTA |
| | | TTGGCAAAACCCTGCATACGTATTTAGCAAATATCTGTAGGGGTCGTCCAATAGCA |
| | | GTGCCGTTTTATAAATTGGGTTGATACATAACACTGAATCAAGTGAAATCGAACGG |
| | | TGGTAAAATGGCTTGAAAGGGGAAGTTGTTTAACATTCGCTAGCGACACATGTTGC |
| | | ATGGTTAGGGTTGCTATTTCGCCTCATTCTCGTTACGACATTCTCAACCAGTAGCC |
| | | CACCAACCCAATTAAGGTCACGCACGAACCTATCATCCACTTACCTCTTACAACAT |
| | | AAAATAGTCAATACACCTTCCTCAATTAGCCTTAATCAAATAAAGCTAGTTATTTT |
| | | TGTCTCCTGGGGATCAGGGCGCTTACTTCGTACTCGCTTCCCCCGCTAGGAAGGCC |
| | | ACTGGTTCCCGAAGAAACGTGAATAATTGCACATGCTTTA |
| 18 | 39.50% | AGTATGGAAGGTGCCTCGGTAATTACGGAAAGAGCTTATCTGCCGGAAACTTTTAT |
| | | TTTGTTTCATCAAAAGGTTATACGATAATACCGCATCTACCTTTTCGTATCAAAAT |
| | | TGGTCCACAAATCCAACTTATTGTCATCTTGAATCACACATTCATCTTTCCGTCTA |
| | | ATGAAGGAGCGTCATTACTTGTTGTATGAAACGCAAATTCTCTACACTAGTAAGTG |
| | | AGACATTAACTACAGCCTATTAAATAATTCAGGTAGACTGATGAGTAATATTTCTT |
| | | CTATATATATGTGATACTCACTCTCTACTGAGTTGACTAGTGGACTCTTTGTTCTT |
| | | GTACACACACAACAGAGAAATGCCTAGAACAAAGTCAAAGAAAGCGCCTAGATGAC |
| | | TTTGTAAATTGCACCAGATCTGAAGTCGAGTCGTGAATAGAACTTTGCATAAGACT |
| | | CTAGGACTTCCGATGGCGTATTATACTTAGGAAACCAAGCCGGTAGTAAGAATCGA |
| | | GGATAATACTCTGGGAAGTCTTCCGTATTTGCGTCAACAACCAGCTTCTGGATCAA |
| | | GCATTTCTTAACTAGATTAAGCTTCCTCTTTCGTTTTAAAGCGTTTTACTTCAGCA |
| | | ATTGTAATCCCTACATTTGTATTAGCCGAATAGAACGATGCTCCTACAACACCAGG |
| | | CCGACCTCATGTTACGATGGCCGAGACCATAACTCTTCGATGAATCATTAGTGGAA |
| | | GAGTTATCTACTGACGGCATGATCCTGGGACATGAAATTGGAAAGCATTTGCACAC |
| | | GTTAATTCGCCTTTTACTTCAACGCTCGGACCCGGTATAAGATAAAATTAGACCGT |
| | | TATCTTCGTAGATCGTAATACGTATCATCTCGTATATGCCGCTTGTATTCAACGGT |
| | | TTCCTTTTTAGACTGGAGCGATCTACGCTGGCTTGGTTTAAGGACTATGCTAGGGT |
| | | TTGTACGTAATCCCTTTAATAATTAACGACCGAGCTGACAAACTGAATAAGTACAG |
| | | CATCAACAGGACGGTTCGATTGACAGCTGGAAACCTATTAGGCATCTTGGCCCTTA |
| | | GCATAAGTCCCAGTATTATTTGTTCCTCCAGTAAAAATCTCCCCGGAATTAGAGCA |
| | | GCGGTGAAATTTATGGACTTGACCTTTTTGGTTTAGTCGTAGAGGGACAAATATCA |
| | | TCTCATCTGAACGCTCATCACCAGTTAGTTCATCCAAATTCAATTAGGAGGCGTCA |
| | | TATTGTCGGGCGTCTGTAACGGAGCCAGATCTAGAAGTTCATTGCTATAAAGAATT |
| | | AGTGTGCTTGGCACATCACCTAATCAAATTTTGGGAAGCAGCATAGCTATTCAGGT |
| | | GTTGGTCAACCAGATAAAGTCTATGAAGAAAAAAACCTGTGTTAGTTCTGCGTATT |
| | | AGTATTGTAGTATAATGTACGACATCCCGAAAGTTAAATTCAGGTCGCAGAGTCCC |
| | | TAGTCCACCGTTCTAACTCACAAATCGATGTTCGGACATAGCTATTTAACAGTCCA |
| | | TATTTACCTTAAGTGTTTCGACTTATGTATGCTAGTTAGGTGTGTGGCTCGCCTTC |
| | | CCACTGTTAGACCACATCTAGACGGACATCGTTAATAATATCTGATATACACAAAA |
| | | ACGTTTACCATAGAAAACACTATATTCATGGACACTTTATCATATTCCTCGCCCAT |
| | | CCTCACGACCCAGATAATAGGGAGTTGTAGTTTTTCTAAACGGTTTTAATATGCAG |
| | | GTCCATAAAGCATGCAGTACATTACTGTTTAAAACTTTAATTCAGATATATCCTGG |
| | | AGAAGAAAATCTCGATTGGTTAATCACTTCATTGTTAAATTCGATTTCGCTATACG |
| | | TTTCTGTACTAGGAAATTTTTCATATTAGGCACGCGGTGTTGGTTCCGTAACACTA |
| | | TTAATTTCCTCCCGGTTCGATCATGGCTTGCGGTAAGTCCTCAATTTAACATAATT |
| | | GAGATACCGAAATCAACCCAGCGTCGCAGTATTTTGAGTT |
| 19 | 39.60% | GGTTAAATGCATGCTCACGCCTCGCCAGGTTGTTAAACCATGTACTTACTCAATTT |
| | | GACAACTGATGTCCACTCTCCACCTCGCGCGATGCTACTTTCTTAATACTAACGCC |
| | | ACCTTGTCAAACACCTAGATCGTTCTAAGTGTAGCACCAGACAGAGTAGACACCGT |
| | | AAAAGGTGAAAAGGGGATTAATTTCTCCTCCTTTTGCACAAAAAAGTTAAGGGGTA |
| | | GGCCGGAGGAAGGTTAACGCGAAGCACCTGCGTAATCGGTTTCGTGCTATATCGGA |
| | | GATATACCGTAATGACTCGTCGACGAAAGTCGAAGGCTTTAAGCTCCATGCCCCAT |
| | | GTTGGTGCGTTAGGACTTTGGTAAAGTGGTAAAATTTAGATCTCTTTGTGTCCTTT |
| | | ATATCAAGTTAGTGTGAATGCTGAGTTTTCTCATTTTTTAATGTAAGTGATTAATA |
| | | TGAAGATGTGTAGTCTAATTTGGAGAACCAACTTAAAACGGAATAGGATCGGTGTA |
| | | TCAATGCATATGAAACCGGTAAATTTAGTCCTGTTGACCTGAAACTGATGGGAACA |
| | | AACCCTCAAACGCTATCGCAACACCGTCCTAGGTTCCATGCACTATTAACCTGTTA |
| | | TTGCCCGTGCGGAGATCTGGTTTTTATTGTTTTATACTCTAGATATATTAGCGGTT |
| | | ATGTTTTTCTGTTAATTTAAGATGCATAGTCTACTTTGACCTCCGGCAACGTGATT |
| | | TGTAGAAAATATTTCCCACACACACTATATGTGCTACTCAGGTTACCCATAGTTTA |
| | | TGTAATAAGTATCACTTTAAACCCTCCACCCGCCCATACAATAGAAGCCCTTCAAT |
| | | TATACGAGGAGGTATTGACCTGACTAGTTTACCAAAGCCAAAGATACCTGGACAAG |
| | | TTGGACAAATACTAAAGGACTACTGTAGCATAGTGTTTGCGGGCCAGTATACGCTT |
| | | ATTTAAACGATACTACTGATAAGAAACACTGGGGTCAACGTGCTTTCATCACCTGT |
| | | CCATTACTCCAACAGTCCCAATTTTTTAAAGAAGGAATTTTCGGGACAGTGAACGC |
| | | GGAATCGCTAATAATATTCAGATAGATAGCTCGACACAATATAACTAATCAGACAA |
| | | AAACTATTCAAAACTTTCTCCTAGGTAGTGCGCGGCTCTTTTACGTGGGGTTTATT |
| | | CACCTGCGAATTATCCTGATGCCCAGGAGCAAACTCATTATAATACCACCAGGTGA |
| | | CAGCCTACAAGTTTCATGGCATGGCTGCAACCTGCACACGAACGCTTATGCAGCAT |
| | | GTGCTCTTGAGTTATACCAGCTACTTGATTCGATATATGGTTTTTGTGAAGAATTT |
| | | GATACCATTGACACGGGATGTTGCAAATATTTAATAAGTCCATGCATACTAATACC |
| | | AACGCCAGAGATAGATTGTCAGTAGAACTCTTGAAGTCAATATGGACCGAGTGACT |
| | | TGGGTGGTTTATCCCACTGTTAGAAAGTTATCGTAAAATAAATTCTTGGTCAAATC |
| | | TAATCCTTATAAACACTCTGTTATTACTCTGCTTCGAATATGTTGTTATTGACCAT |
| | | GCTGATAACTACATCCTTTATGTTAATTCAAGGCATTCTCTGAAAGTCAACAATTA |
| | | ACTTCATATCAGACATTTGACCTATTCCTCACTTTTCTATAACATGACAATCACGG |
| | | TGATTAAAAACATGACGCGTATCGGCAGCAAACCACTGTACTGATATGTAAGAGCG |
| | | CCCGTCGCATAGATATTTTAGACTCTGTCCAAATCACTCTACGCCAACTTGAGGTC |
| | | AGAATGCATACCGTGGTAAGCTGAATAGTTCTTATACACTTTCTAATTTACCCAGA |
| | | TGACGATTTTTTGTTATATGAATGACGATCTTGGCATTATACTGCCAAGACTGCAA |
| | | TCAAATCCTAAATTCATAATTTAGTAAGTCAATAGCAGATCTGAATCCCATAAATG |
| | | AATTCTATCGAAGTACCTACACTATGTCACGTAGAACAAG |
| 20 | 39.60% | CTAGGTAAATTCTTAGGTAGCCGAGTTGAACTATTAATAAGTCTCGTCTGTGAGTA |
| | | TGTCTTCCGTTAGGTATTTTCATATAGCTTCATGTGCCTGTAAAGACAGAAGTATA |
| | | ATTGGATACATCAGACTTTTTATCCCTTTTACAGTCTAGAAAGACCTACTTGAAAC |
| | | ATGTTTCTTAATGGGTAACGTAGTGAATTATGCTCGTTTTTCCTTTGGTAGAATGA |
| | | TATTTATCTCCATATGCTCTGAGTTGGATAATTTGTAAAGAATTATACACGTTAAT |
| | | TCAACCTCTTTATCAATGAACTACGCGGGCTTGATCAGAGTAAACTCACAATAGTA |
| | | TCTTGATCTTCACAATCTGATGGATATTGATGCGAGTTATACGACCTGTGGCATAT |
| | | CAACAATGAAGTGAAGTGTCTGTCCTTATGATTCGAAACAAAATAAGTGTCCTTGC |
| | | TAGCTACACCCACACCGCGGTGTGCATCCCATAAAGGCTCAGGTATAGTCTTGTCA |
| | | TAAGCGCTACACTGCCATTCGTTTAGAATCATTGTTTAGCAATCTCAAAAGTAATA |
| | | ACATCCGACTTTCGAATAGGTTCAGTTTCCTGATCTACTGGAGCCTATATATATGC |
| | | ACAGACGAATCTCGTACATGGCATAAGCAAGTCATGAGAAGAGGCTGTACCACGTA |
| | | AATATAAGCCTCTGATTACGCTGAAGCTTAATAATCATCACCCATCTACGAATCCG |
| | | ATTGAGGGCATAGGCTTTCATGTCTTTTTCGCTGTAGGTCTATGCGATTGTGAGAC |
| | | TATTGAGTTTTCCACAATATGGTGGTAGGTACTGAGTAGGGTACATTTCACTGTCC |
| | | TATTGCGCTGTCGTATGTCTATCCGCCGTTGCCGTCGTCGATGTTATACCATTTGA |
| | | CTAACAGTGTTATGAGTCACTCCCTTGGATGCGATGTACCTTCTGTTGTGAGGGAT |
| | | GTAAGTTGCAGTTAAGCACTATTAGCGAATAACGCTAGGATTCTGGAAGAAGAAAA |
| | | CACAGGGTCGCTTCAGGTCTCGAGAATCTTACGGTTAGAAAATTTGGATCTGAATA |
| | | AAGAGATGTCTAGCCAGTGTGGGGGTTGAATAAGCTAAATGTCTGCAATGTGTATG |
| | | CTTCTGCACAGATATTAACAAATCCGCCATATTTAGGCACATTTGGTAATGGCTGA |
| | | CAATCGGATCTCAAGAATTCTATACTGAGTTATCGGACTACAACTAAAAAGATGCT |
| | | ATATAAAATTGTCATAATTCATGAAAAGCCAGTAGGCCGACCATCATCGCTCTAAG |
| | | TTGAGTTGTTTGACGCGAGGCAACATTACGTGCATGGACGATATACACGTTACTAG |
| | | TTGTATGGTATTTCGGCTAAGTTTCCTAGCTAATTTCATTAAAAGCTGCGCATTGG |
| | | TGTTTTTCAGCCTATATACTGACGTAGTAAACTTACATACTTAATTATACTAGGTA |
| | | ATGATATAGAAAATGGCTGTACATCCTTTCTGAAATGCTTCCATGCAATGGTGCTA |
| | | CAAGTCTTAGATTTACATTATAATCGGAAAAACATCAACAGTATGATTACCTAGGA |
| | | GGAGCTAGCATATCCAGAAAGTAGAATAGCAGAAGCCACCAACAGACTGGGTGAGA |
| | | GTGACGTTATGACGGATGGATCATACCCCATCTTAGGAGGGTCAGGTCATTTCTCA |
| | | ATCATATGTTTCCAGATGCGATGCAAAGACAAGGCCCAGAAATTTCAATTGTAGGC |
| | | CAATCGTCCGGTCGTATTAATCTCAACCAAGTAAATAAAAAGCATGTGGGCTGGGC |
| | | GCAGTCAAAGTCGCTTTTCTTGGTCCTTACTAATCTGAAGAATATACAGTAAACAG |
| | | AGGATAGTGGGGCTAGTTCAGAGTAATAGGCAACAAACCCTTTCATGCATTACTGT |
| | | AGAATTTGATACTATTGCGTGTATCGCTTTTAACTTTATAAAGAGTCGATACAGCG |
| | | CAGGCTCATAATGTTTGGAGTCTGTCTAATAAACATCTAA |
| 21 | 40.50% | TTTATCTATTTCATATATTGCTAGATAAAGTTGACTGACTTATTACGATTATTGTC |
| | | CCAGACAGCCGAGCTGGGCCGTGCGTCAATGCACGGGCTCAGCCTCCTAATGTTAG |
| | | CATTTGTTACTCTTGGAACATTTGGATATAGTTGATTTTTTGATAGTGCAAAGGTT |
| | | CTCGGTCATCCGGTATAACGATACTCCCTACCCTAGACATTCAATCGGTGCGATGG |
| | | TAAGTCCGTTTCGCACTGAAAGCCTGTAGAGTCTATTTGATGTTTACTTAATGCGA |
| | | TTTGACTCAAATGTAGGTTAGGAGTCCGTTCGCATCCTATGCAGTGATAAACTATC |
| | | TAGTGTGTTTAAAAAGACGCAACCACTACCAATCAGACCAGCAAATTTACATCAAT |
| | | TTATGTCAAAACGCCCTTACTTCGTCTAAATATAGATATATCACCACATCAAGCCT |
| | | GCACTTCTCACACTATGTTCTATGTCATGTCGTTGTACCGAACAATTGATATTTAA |
| | | CCGGAGTTGAAGATCAGCTAAAGAGAGAAGTTATATAACCAACAAATACAGCCCAC |
| | | CCATCAATGATCGTGAAAACAAACTGTACTTAACAGTTCAAGAACAGTCACCATTT |
| | | CTCGACGTACAAAAGATTCTTCCATTATGGTTCGATACAAATTGTTCAAACGCCTG |
| | | TCTATAGCAGGGCTCCGCCATATTTCGAGCATACTAAATCATTGGGTGGTCAAACA |
| | | GTCTCACAAACAGGTCTGTTGCGATTCATACGAGACGACCATACTTAGGCGTTGAA |
| | | ATGTCGTTGCATTTAAGTAACAAATACTATAGACCGCTGGTAGTCGCCATATAACT |
| | | CTGGCTCCAGATTATACATGACCTGTTTAGAAAGGCAATGGGAAGAGGGCAAAACC |
| | | CCAAGATTGTTCCTAATAGTTGTAGATAAATGGATGATATCTGCATCATCACTGTT |
| | | TAGAGAATCCCGCTTTCCTTTATTCGGTTATACTCACCGTTTCTCGGCGGGTTGAG |
| | | ACATGCATAACTTCTATCTATCGTTGAGAATTATCAACTTCAATTCCCGAGACTGT |
| | | CATTATCTATAGTTGAGGAACCTTCGTCGCTGCTATTGAATAGTAAGAACCCCTCT |
| | | AGTCCAGCTGATGCTTGTGGTAACTGCACTAGTAATTCATCTGCCATCCGTGCTTA |
| | | ATTGGGCATGCTTTGTTGCATCCCACTCCCGAACTTGAAGGTTGGAACTCTCGTTT |
| | | TGCCAGCACAGTTAACAGGGAGTAAGACCTATTGGTGTGACATAACAGTTAGGTAA |
| | | ATCCATCTAAACACGTGTGTTTACTAATATTCAGTCGGTGGACTAACAGACAGGAG |
| | | CTTACCCATCCGTGGATGTTTTCTTAAGGGTGTCGTTAGAATGAATAGTACATGTA |
| | | TAGTACTGTCCGAGGTGTAGATAGAATAAATGTGACCGTGATCTCAGATTTATGGT |
| | | TCAAACGTTCTAATTTTCCGAGGAGTAGTACATGTTGGTACCTTTTCACATTATGG |
| | | TGCTAATTAGGCATGTATAATATCATATCATAGCTTTGCCCATACTGACTATACTA |
| | | AAATTGCTATTTTGGAAAGTTTATAAGGCCGTTTCTCATTGTATCTAAGACCTAAG |
| | | CTTCGCGTCAAGAAATACCCTTACAATCGGCCTATTTAAAATTATTCATTTGTCTA |
| | | GGGCGCGATGATCCTTTCCGAATATTTTATCGATTACTACTTATGGATACCCGTTA |
| | | GACGCTTATCCTCCTACTACACCGTACTAATTACGTACTTTTTTCGAAGTACGATC |
| | | TGATTAGTGTCGACCACCTTGCCCTTAAATCTGATCGCTCCCACCAGTACGCAGGA |
| | | CACACGTAACGGTTTCGATACCCAGCGAGATCAGCCTTACCAGTGCTTGTGTGGTA |
| | | TAACCACACTATTTCAATGCACAATGACAAGAGTACTATGTTAATTCACATGCCTA |
| | | TCTAGTTCAATTACGTTCAGACTCATAAAATGCCATTGCT |
| 22 | 42.00% | TCGAAATTGGATCGACGGAGCTAATACGCAAATTATTTGTTTGTGATTTCTATCGC |
| | | GCTTCAAAACCTACAAAAAATAACAGCCTTTGGTGTAATTCGTCGTGGCCATAAAT |
| | | ATGGCTTATTCTATATATCCGAGGCCCAGGCCATAACAAATTCTCCAAGATTTACT |
| | | AAATTAGTACGGCCTTCATTCCGACGGGAAGTTTAAACTCAAGCCATGGAGTCCGG |
| | | TAGTCTTTCAACTTTGTCGTATGACGGTATGCTACATGCCCCCAATCCGCTATTGA |
| | | ACAATGGCAAACACTACAGCAGTTAGCCAGAGAATTACGCTCTTTCACTTTCCTAG |
| | | AAGTACACAAGTCCTGAACCTACCAACTGACTGTACACACCCTCTATGGTACTTTT |
| | | GCTGTTTAGTTGCCGAATGATGCATCATGTCTGATTTTTCGGGCTAGCCTTAGCTG |
| | | AGTGTCAGCTTCACCCTGATAAGACAGGAGTCAGAAACGGAATTTCATTAATACCG |
| | | CCTAAGGCGAAAGAGAGGCTGTCATGTAAGCCGGCAGGTTTCCCCCTTACGGGGCC |
| | | CACACTCTCCCCTCGCTATGAAATGACACTTCACAAACAGTCGCTACTCAGGATTT |
| | | ATTCCAAGTTCCAACGATGTTGAGTACATTGAGAATGTATTATATTAAGCTAATAG |
| | | GCAGTTTTCTCCAACTATCGATTATTCGGCTGATATAGCCCCCATCCTGAGACGTT |
| | | ATTACGTCACTGAGGATGATCTATTCACACAACACTTGGGTTACCATAGTTCGGAA |
| | | TGCGATCTAACGTCTCACAATGGTTTTTGGTGGAAGTATAGTCTTATTCCCCGGGC |
| | | TATCGCAAGCACCCAGGAGTAGTTTCGTTGGTGTCATGCTTATCCCTACGACCCAC |
| | | CAGAGTGTCCAATCAATTTACACCTAAACTGGAACCTAATATATTAATCAAACTTT |
| | | AAATCTCTATATATTCAGACTACTTTACTCACTTTGATGTTAGATGCGTAACAAGC |
| | | ATATAAACCCGTTTGTGATCGTACTCAATCGCACCCTTCTCGTTATTGATTGATCC |
| | | TTGCGCGAGGTAACCTGGGTAATCTCTAAGTTATCGATGCACCGTATCAACATTCA |
| | | TGATCGAAAAAAGTTTAGTGAGAAGGAGTTAATGGATCGTTCCGACTAAACTAATG |
| | | GAATTATGTATGGGATGTATTTCGTTTGAGCCAATTAACTAGGAACTAACTCATAC |
| | | ATCTTGCAATAGTGGTAGCGTAAAATGGTTGAACGTAGTTGAAATAGTAGGGATAC |
| | | GACATGTCCCCTAAGCCTCACCCTTGGTAGTTCTCGTAAGCGGACAACGCGTTATC |
| | | ATCACGCTTTGGAGTGTACTAGTTTATGTCTACTGCGTTCGCTGACAATAAGAACA |
| | | GCAATATCCCAATTCTCAGTACTGACGTAGGACCATTAGCGCTATAAAAAAAGTAG |
| | | CGTGAACTGTCATTTATTAAGCATTCCATTTTATCCAGTGTCCGCTAGGCGGCTAA |
| | | ATTATACAAACAGAACGGTGTTCTTATACTGTTACTACCTCCACAAGTGGGATTTA |
| | | CGAACGCAGAAAGAGATAAGCTCACTCTCGCTATGTGCACCGATGAGTCATACAGA |
| | | GGTCATCAGTAAAGGAACTCAATCTAGAGTTACAGTCCAGCAATCCAATCCGGATG |
| | | CCAACAGGCGTAACGATTATATTCAACCACTAAGCCGCATAAAGTATCGATGATTA |
| | | GCGGGGGAATACCTCCTAAACAGTTTGACCGGAACGTCTACAATACTTTGCCGGTT |
| | | ATCAATGAAATATGCGGGGACGAACCATGCATCGTTACTCAGCCTTTGGTGTACGC |
| | | CAGTAGGAGTACTACTTGTTCTTCTTACACGACACGTAGCTACTTCTATGTATAGT |
| | | AATGTAGTTGACTATAGAATGACGAATAGAGAAGGGAACCAGAGCTCACTTATTCC |
| | | GTCAACTCGATTTATCATGTTGTTAAAAAAGATAAAATGT |
| 23 | 40.00% | GCTACTATTTTAGATATGCATCAAAGAAAAACAAGGACATCTCCTGTATACGTATA |
| | | GGTAATAAGAAGAGGATCCAACGGAAAAAGCCACCGGTGGAGATAATAACTATTGT |
| | | TAGCAAGTCCAGTTTTCTGTCAGGGGCAACGTTAAGATAGAGGCCAGGGTAATTAT |
| | | TTAACTACTAGCTGCACTTCGACTTCATTTTCTGAGCTCTGTAAATACCAATGGAG |
| | | CGAGTAGCTACGGTTAAACAGATATCGGCTGGATGTCGGTGGTAGGAAAATGTGCC |
| | | TGTTGCGGCTGATAAGCATTAACTTACCTAAACATAGATTGTTGGTTTTCCTAAGG |
| | | TTTTATAAGAACGTATATAAAGATTTCTTAAATGACAAGCTTAGCCTGCATAGGCT |
| | | ACATGTGAGTGTGGATGGCTTCGACAGTGATCCCGCAGTGGACCAGATTCCATTAC |
| | | CTGAATGAAAACGTTCAATTAAACCACTTACCGTATCACTCTGTCCTTGTAGCCCT |
| | | GTAAAATGAGACTTGCGGATACCAAATTAGCCAAATTATTCATCTAACTATAATAC |
| | | TTCTTCCATGAAACATTAATACGGCCACCGGGAAGCCACCGATTCTGTCGCCTTAT |
| | | ATTTTTTGCTCTATGTCTTTCTTTTAGTCCGACAACTAATGTGAACAAATTTCGAC |
| | | CTAACAAAATAGAGACAAATAACCCTATATTAATACAACGCTACGAAGATCTTCAA |
| | | TAGGATTGGTCCGATTATAGACCAATTATACTTTTACATAATATGTACAAAACATC |
| | | TCGGCATTCGATGGCATTGGCGTGGATATTCGATTGTAAAAGCAATGGATTTTTCT |
| | | TGCGCTGAAAATGATGATCGCCCTCGATCATCTGTATAGCACGGGTCGAAGTTTCA |
| | | GAAATGATAGTTGCTCAATTTGGTTCACTTCGAATTTACGCTGATGTCCCAAGCGA |
| | | CATGTCCCCGATCAACATGGTTGTTGGATATCAAAAAGCTGATAAAAAATGTGAAA |
| | | GGACACGCCTCCAACGCGTAACTGTTTCACCTACTTCCATTTCGAGGAACTGGGTC |
| | | GATTTAACGACATCAAAGTTGTTTGCTCAGACAGTCTTCCTATGAAAATGAAAAGT |
| | | GATCTAGGAGTAGAACCCGATGGCTATTAATAAACACACTCTTACTAAATAATTTG |
| | | GCGAGCATCAGAGCGTAGGTACTCGGAACCTGATTGCCGTTCCGCTTTCTATACAC |
| | | TGTGAATAACAAAGTCATTGAGGTGACAACCTTGCCGCGTGCACGGTCTAAAGCAT |
| | | GAAATTTTAAAGCAACAATCAAATCTCTAACGGCCTATCTCAAGTTACGCAGCTGG |
| | | CGGTAGGTGGGTTTTCGCACTGACTCTTTAACCAAGCTGCTGCTAAAATACTCTTA |
| | | CCTCACTGTTGATATAATGGTCGCGATTACAGATAATCCCGCACATCTGTCAAATA |
| | | GAAGATCCAGTAAAGAGTCCAAATCAGAGAGACCCAATAAAGTAACCAAGGCATTA |
| | | CCGTTTCACGAGGTGGACTTTCATGAAAGCATAAGTATGGCGTATAATATAATGTT |
| | | ATTTGGAAAAAAGATCTCCACAACCTGTTTTACCGCTGAAAAACCTAAATACCGTA |
| | | CCAGACGAACCACTTGATAGTCGAATGCGCCATTGAAGGAAACATTCTCCGTTAAT |
| | | CTGATTTTAAGCTCATCAGGCTTTTATCTTTGCGTTATCTACATTTGACGATTACC |
| | | AAGGATCAATTACGTGATTGGACTATACTTAATATCAATGTACGAAATCGTCTACG |
| | | ATACTACAAGGTAACCACTGATAATTCCTCATTGCTCTATGTTCACACTGACCTTG |
| | | CTAATCGACGTGGACTTGCGTCCTTGTCTAGCTTATAATAGTGAGATTTAATGACA |
| | | ATGCTGGTATAATACCGTGCAACTACACGCATAGAAATTACTCAGCGCTCGAGAAA |
| | | AGTAGATTACTTCGCTCCTTCGGAGTTTTGCGTATTTTCA |
| 24 | 41.00% | CCTCATTTGCCCTTTTATATTTACCCGAGTTAGTTCACGAATGTGCCATAATTCTG |
| | | GTCGCAGCAAACTGCGGTGTTTAGAAATAATCTTCCGTTATTCGTTTATCAAGACC |
| | | TCGTTGTTTAGTAGTTCTAGCTGAATGCGGTCTATTAAGTTGGAGAAGATCTGGGT |
| | | TCATTACATTAGAACCCAAACTAATTATTAAGTTCTGCTCATTAGCATTAGGTAGA |
| | | ATCTATTCTTGTCCGGCGCTGTTGCTACTGGGTTTAGTCTAAGTAGTACTTTAACT |
| | | GTTCCTAAGGGATGCTGCAAAATGAGATATACTCCTCCGATAATGATCAATTTGGA |
| | | TTTTGGGCAGCGGTAAATGTTTTATAGTGTGAATTGTGTTACTAAATTTCATGACG |
| | | TAAGCTGACCTTCTAACCGTCGTGCTTGGAGGATTTACGCGGCGCCAAAAAGAAAT |
| | | ATACTAGTCCCAATCGCACTAGGATTTGTTTAAAAAAAGACGGAAAACCTGCAACC |
| | | AAAGGTGTCTTGTACTGACTCTATCTGCAAAATTTGGATGTTCTAGCTCCGTTTAT |
| | | GGTCGCTACATGGAAACGCTATTGGTTAAAGATTCACTATAGGCCAGTTCAAGTTT |
| | | CCCGAAAAATCGTGACGGACGTTATACTCTAACATTGATAAGAACCATGTATCAAG |
| | | CGATCCGCAATATAGGGAAACACGGCGAAGATCAAATTTATAGATGGGAGGAAGCA |
| | | CACACAATATGAGTATTAGTGTGCTGAAATCAGCAGCGTAAAGTGCTTCTGTTCCA |
| | | CCTATACTTTTACGAGTCTCGTAATAGCGTATTACCATGTAAGATGCATTAAAGCT |
| | | ATAACTTTATGGCAAAAAAGGTAATTTATTCGCTCATTACTATTATTTGTCGTTTT |
| | | GCATAAATAAAGTGTTGTTACTTCAGGAAGCTTTAATTCTCTGTCTGCCTTAACCC |
| | | GAATTCTACGCGATCTCCGTATAGCAGATGAGAACCGGTGACACGAGACCCGCACT |
| | | CGCAAGTCGTTTCTTGAGGCTAACGACAAAATGAAGCCATCAGCGAAATCTCATCC |
| | | GTTAGGCTACCCAAAGTTAAGACTTTCCCTGTATCCCGCTAATGCGTCAATTGGTA |
| | | GACGTATCGGGATTAGATATTCAAGACCAAGTCAGGTAGAGTTGGCGCTAGTTGAA |
| | | CATGGACCTGGCCTTACAAACAAGAAGACCACGAGAGCCCTAGTACAGGAATTTAT |
| | | CGGAAAAAATAAGAAAATTAAAATCCCCGATCTGTGTGGTGCTCAAATAAGGCAAG |
| | | GGCGCTTAGCCTCACAGTCGTTACTAAGTCAAGGTTCTAAAAGCACGTGTTTTAGC |
| | | TTGATGGATCATGACTTCGCTACGGTCACTACTCCACCGTGTTTCTGGAGGTATGC |
| | | AAGGGAAAATCGAGGGATGTGCTCAAATCTGTGGCAACCGGAGCACCATTCTAGGT |
| | | AACTTCCATTAACTTTTGATTTAGAGTATATGGTTAAGCTATTAAACGTTTCCTAA |
| | | GGACAAGTGGGATAGTGATATACTTTTTTCGGCGACATCAATCCAGGATTATCCGC |
| | | TAACAGATCGCCTAGCGCTACCGCATATGATGATATCCTTAGGAAGAGATCCACCC |
| | | CGGCCAAGAAACTCCACACTCAATAGGCGGTGACCTATTTGTGAGTTATGCAGATG |
| | | TGTTTCAAGACTCAACGCCGACAAAGTTCACCACCAGAGAGTGTAAGGCTTATCAA |
| | | ATTTCTGATTTTATCGACTTATAAATTTGACACGTCTAACAGATTCGGCCTTTGAT |
| | | TGTAAACATCGCCGCTATGATATTTTCGTGATCCTTTGGGATACGAGATGCATCAG |
| | | TACTGGCCCCGAATATTTCCATTTTAATTACTGTGTAATGCTTAGGTTCACAATCA |
| | | ACAAGTAGTTCGTGAAAATGTTACTATAATATCCACACAAAGATTTACGCACTCTA |
| | | ATGGTGGACGTTGGACCTCTGTTAACCCGCTTTCGTTATT |
| 25 | 40.00% | ACTAAAGTCCTGGAGAGTATGCTTGGCCTCGTGCGGTAACATTTGAACAGCATGCT |
| | | AGGTGCTAGTAGACCCTTTCTTGACAGCGGAATTTGCTGTTATTCAAACCACCTGT |
| | | CAGGCCAATTCTGGAGCGCAACCCACAGTGATAGAAGATAGTCGTTACAATCAAAT |
| | | CCCACAACTTGAGACTAGCCCTCAGACTGCAACAGTACACAGTTATGCTGTGGAGA |
| | | CAAATAAAATACGTTATGTATTGGTCATTAGATTTGGCTTTCTTATACGTCGTGTA |
| | | GTAATGCTTGTGATCGGTTGCCGACATGGTTACGAATAGCTGTTTATTAATTTAAA |
| | | ATTCAATTCTGTCGATTTAGAGGATGGATAATATCCGCTATGTAGACATGAGTGAG |
| | | TTCCTTATCCTTCAATTCCCTTTTTTCTGTTATTTTGGATCTACGAATGAGGTATT |
| | | AAGTTCGTAGCACTCGTCCGTTTCGTGGAATGACTTATTCGAGATGGCTTGATAAG |
| | | GAATTGTACCTCAAAGGTTTCATTGTTAAGAAGATGAATTTTCACGCCCATGGCAT |
| | | AAGCATATGATTACGTCCACTAGGTCATAGACACATGATAACTCGTCGCTCAAAAT |
| | | AATCGAAAGAACGTCTATCGGCCAAATTATTACTTTGATCCCAAAGGAGAAATCAT |
| | | ATTGGGGCGCGGGACTTCATGTGTATTACCATCCAGCAAGCATTTGATAAAAGTAA |
| | | CTCCTATATTATTATGAATAGCGGTAAGTTTCTTTGACCAACCTGACAATAACACC |
| | | AAGTGACTCACTGAGCCCGTTATCTACTAGGTATTCGCGAATACCGTAAAAGCTTG |
| | | ATGCAGGTGACAATGAGAATTATCATTAGCGTACTGTATGCTCAACCTAGCCTCCT |
| | | TGCAAGATTTCGTTCTATCTATTTTGTATTCATTTCTTTCCGCGACATGCATTCTT |
| | | TTGCTAGATCCTGGGTCCTGCAATCATTTATAAGCACGCAACTTAGCTTAAAAGTG |
| | | TGGAGACGAGACGTACAATCACTACTTCCCATCACTTCTTCTCTTATAAGCGTACC |
| | | GAAAGACCTCGTATTTTATTAAACAATAACGTGCAGTTGGCCTAACATAATTCGAT |
| | | GTCTTTCAGTGTTCTAGGAAAGGTGCGGTGTGTCTAGCAAGCATGTCAGCCCTACA |
| | | GATTCTTAACATACCTATGTGTCTAAATCGAGTATACTATAATGATGTACCATAAG |
| | | CCCTTGCCAAAGGATCATATTCGGACTAGTTATTGCCTTCTGGATGGGGTACTTAG |
| | | ACTAACATTTTAAACCTCTTGCGATACGACCTGGTGCTAATACACTATTCCTTCTT |
| | | TTCTCACGCGAACTTTCAGTATCGTACAAAAGTATGGGATTTAAACCTTTTGAAGT |
| | | TTGGTCGTGATTATTTGTTTTTAGGGCCTCCTCGACGCCTCAAATAGGGATTTCTT |
| | | CAGCACTACATATTTTGAGCCGTATGCGAACCCTTCTTAGGACCGCGGTAGTTTGT |
| | | TCACGAGCACGTTGGCCACACCCCAATTATCCAGAAAGCCGGACTTAAGACATATT |
| | | GAGTTTGTTAGTGCATAAATAGGGTCGCATATTGATCTGCGACTCGAGTAAATGTC |
| | | GTACTGGTGATATATTCTCCCGTTTTCGAAGGCCCCAATCAATTACTAATTACCCT |
| | | ATTTACGAATGTCGAGAGATGTTCAAACGAAACATGAGGGCGCATCCCAACGCCCA |
| | | TTTTGAAACTTGATTGTTGTATAATTCTTAATTTTTGTAGATTCAGCGTTCTTGAC |
| | | ACATTTTAAAGACGTCAGTTCACCGTACCTACCCCTTCGGTTACGCGAAAAAGATT |
| | | AGGTTAACGATTTCTATCGTTCGTTGGTTGTTATTTCTGCAGTACATTAATTTTAT |
| | | AACTTGATATATCAAATCTGTTTTTGATTAATGTTTGAAAGCAAATCGTAACACCA |
| | | AGGAATGCAAATAATCATACGTGGCGGACCAGCTACTATA |
| 26 | 40.10% | TACATCCCCATCAGTCAAGACGATTCGTTAACAAATATCGCTGACTGGGAGAATCC |
| | | CAGCATGTCTTGGCTGGCTAAATAGAAGCTACTATGTTACGCACTTCCATTTTGAA |
| | | TTACAGGCGACAACATTACCAGACTTAGTTAATTATTAAACAAGATCACTTTGCGA |
| | | CAGTCCTCTGAGGATCAGTTAGAGTGCAATCACTTAAGTAATACAAAAATACAGAA |
| | | GGATTCTCTGGCGAACAGGTTTATTAGCGCATGGCCAAATTTCTAATCAACCCCTT |
| | | TAGTTAGTACCCATTTCTAGCCAATATCAAATGTACTCCAAGCCGGCGTATAGTTG |
| | | TCAGTGTGTGATTTAACGAATAGGATCCCCCCCCATAACAAATACTAATAAGAGTG |
| | | GAGCAATTATAGTTTAGATCGTAAAGGTTTAAATAAATAAACGTCAAGCACAATTA |
| | | TGGACTCGTATGGGGACAAATTGAGCCTACTAGCAGTTCTAGCGAAATAAGTTGAC |
| | | CTAACCAGTCCATGGACTGCCGGTTCGTTGAAGTCGGTCCAACGGATTGCAGATCA |
| | | TTGCTAGGCAGTTGGTAGATAAATTTCTAGTACTTATAGTCACGTAATTGTCAAAA |
| | | GTCCTACGAGCGTGGTCACCGTATTACTACGACCTCCATAGTTTTCTACCGTGCAT |
| | | TCTGAAAGAAATATGGCTGGAGTGTCCTAGCTCATGATAGAAAACGCCTACACTTA |
| | | GCCAATCAGACATTAATGCGGTAACGGATCAAGCATTACAGGGCGGATTGGTCGCA |
| | | TATCATTGCACGGAAAGCGTTGCCTTAAGTTCGGTACATTCCACTTTCAACTTCAT |
| | | ATTGACTCAAATAGTGGGACAGTGATTTACGCGGAGTTTTAATCTAAAAATTCTTG |
| | | AGTTTATGATAGAACAGATCTAAATTACGGTTTTTATATGTAGTGGTATTAATAAT |
| | | GTTCATAACCCTAGATATTTCCGAGATTAGCACTCGTTCGGCGCATTGCCGGTATA |
| | | GAACAATATGTGAAGAAATTTGCACCTAAGAAGTTGATATTCTCCTCTACATGCGT |
| | | ATAATATATAGTACCATAAGTGGATCATTATTAAAATAAATCTGAGTGGGTGGACT |
| | | TATCTTCTGTCACCCTAACTGGATCAGCAGTGGGCTAGTAGCCATTAAGGAACAAC |
| | | CACTTGGCCCGAAACTATTTGAAAAGTGATAAATACATACACGATTTACTACATAA |
| | | CCACTCCTCTTGTTGATAGGCATGCCCAAGGATTCGTATGGGCGATTTTCCATAAA |
| | | CCTACAGGGTGATTCGCGCATATAAATAACACCAAAGCAGTCAGGCTTTTTGTATG |
| | | AAGTGTAGCTTCCCTAACAGTATGATAGTTGTGTAGAGTCGCTTCTGAACTGGCTG |
| | | ACCCTAGTTATAATTAGTTCGGCGGAGGATGGGCCGCGAGACAAAGTATACTCGAA |
| | | CCTTAGGGCCGCATTCCAAAGGTTATTTAGATAAAAGTACGCAAACCCGCACATGA |
| | | GTTGAAATAATGAAGTACAATGTTATTTATTGTGCGTGGTAATAGTCTCGTGACTG |
| | | AAAATTTTTACCTTTAGGGTTCTCTATCCGGAGGAGCGTCATGAGCTCAAATACAA |
| | | AATCGGAGCATTGACTCAATTACTACTTTATGACAAATTCTACGTCTAAGCGATTT |
| | | TTCTAAATCGCCGTGATCAACAAACTAGATCTACACCAGTGATGCATGCTCACGGC |
| | | GAATGTCCTGAAGTCAGATCTAATTCTTAAGGGTTGGATTAGCTGGCTATAGCAAG |
| | | CCATATTAATATGATTAGTCGTGTATGGTTTACGCTACCTCTCCATAGATATTTCT |
| | | AACTTACATTTGTAAATGTTTCCAAGCATACCGTCAGTATAAATACCCAATGATGT |
| | | GGCTCTCCTTCAAGTGTTTAGATAATAGCTATTTCCATAAGGTGCCTCCCCTATCC |
| | | GCTCATCCTCGGGTTTCATATGTTGTAAGTGGCACTTAGA |
| 27 | 39.50% | TTGTTTCTTGGAGGGTTACTTACGATTATTCAATGTCAAGCTGGTACCAAATAATA |
| | | TGTTAACATCGACAACCTTGCTGATTCTTTAACTGTACGATTTACTCAATCCTTAC |
| | | AACAGTCTTTCCCCCCGATGCTTCCGATAATCCGGATGGAATGTAAAAGCTTTAAT |
| | | TTAGCCATAATGGAGCTACTCTGCAACAGTAAGGCAAAATTTTCTTAAATGGAGGC |
| | | CAGGCAAGATTTGTCCCCGCCAGAATAGCCTACTCCACAATATTCTCTTTAAATAT |
| | | TCGCCATGCTATCTCACGCATCCATGAACAGGTTATGAAAGCGTAGAGTCAAACGT |
| | | ACACTTTAGGTTAGGTGCCTTGTGGGGATTTCACGCCACAAAGTAGAGTAGAAGCA |
| | | GTGTATCAAACTATGTGTAAAAGTAATTTCATATAGTAATAGCCACCAAGAATGCG |
| | | AACATAGGTGTCGGCCTGAAGATCTAAAATTATACTTATTAACAATCATGTGAGTA |
| | | GGTTGGATTTTAACACGTTCATAAGTATCGATCGCTTCGCTTAAATAGAATAAAGT |
| | | ACACATCATGTGACGACGCGCTTCGATTATTGTGCTGCGTTAAGAGTAGTAGGATA |
| | | ATTTTTGATAGACCTGTCTATAACACGGTATTTAATCCGAAGTTCACTATACAATC |
| | | ATAATAGGATATCGTGTTCTGTCTCGATGATCTATTCGTCGCTTCGGGTGCAATAT |
| | | AGGATTCCTATATGAAACTCACTTCCCTGAGCATTGGGATTTCTTGATAGCTAGAT |
| | | CGCGTTAGAGTCGGGCGGTGTATAGTCTCGGATACAAGAACATAAGAGTAATTATG |
| | | TGGAACCTTTTCATGTGATTGTGCTAACTGTGTGATATTCGCAATAATTCCTACAT |
| | | CTTAGTTTTTAGACTGGACTTTTTTTTCCCAAGCTCTAAGCATACATTATTCGCTG |
| | | CGTATGTCACTGACCTAGAGGAATAAGTGTTCTGCTGTCAAAACTAACTCTCTCTA |
| | | GCAGCCTTTTTGACCATATTATCAATTACGCGCCATCCCATAATAACTTCAAAATT |
| | | TGCAACCATCGGAATTAGAAATCCCGACGTAATCAAGACGAATCTTCGCCGATTAT |
| | | CGAGCTTACATAATCGAAGGTGCATTTCTGAACCTTGGCTACGCTAACCCTCTAGT |
| | | CGGGGCAAGATGACTTGGTTATCTGGTTAACTAGGAACTCCTAGCCTCATATTGTA |
| | | TCAATCTGATCTAATACAGCGTCTACCAATTATTTGATTAGGTTTGCTTGCCCTCA |
| | | TAGCATCGCAGCGAGTATCTCACAATGTGTATGGGTATTCTTCTAGTTACGAGTTT |
| | | AGACGGAGAATAAGCCGCTTGTGGTTAACCTCTGTAAATACCTCTAGTTGAATAAG |
| | | TGTGCAACCCAATTCACATTCGTCATGTTAACAAATCGGCAATCTTTCCACTAATG |
| | | AGAAAAAACAAATCATTAATATATGTGAAAGTAATTATTGTGTCCTCATAACGGTA |
| | | AAGACTTACGAGTAGGTAACAATCTCAACTTCACCAATTACCACCTAGATTCCAGC |
| | | ACCGCCAACGTAATCAGTGTTCCGTGCGTCTTACACAAGAGAACTCCTTAAGCGGC |
| | | TAGCGTATACTTTTAAGAGCAGTGGGTATGTGGCCCGGGGCATCTATTGTTTACCG |
| | | TAATATAAGCGCACTAGTCTATTTTTACACTAAATATCATTCCATATCCGGTTCTT |
| | | TCAGTAACAAAAGTAAACACAGTGTTTTGGAAGCAGTGTATCAAGAATTGTGAACT |
| | | TCTTTCACCGGCGCAGGGATCCACTGTCTAGAGAGAATCTTAATTCTATCAACCGA |
| | | CCCTCCATGTCTTATAGATTGTGTCAACGGAGCACCTAACCGTATCCTTAAAAATT |
| | | TAGAGGAAATAGAACTCTCATTCTTCAGCCTGTTAAGCCAATTAAATCGAAACCGT |
| | | TGCTATTAGGTGTAACGGTAGATGTGATAAAAGGGTCACA |
| 28 | 40.60% | AGGACGAGCTCTAGGGGTGCCCCTGCTGTTGTTGGTTATTTAAAAGCCGCGATGAA |
| | | GAGAACGCTAGGGGGAAAAAACGATTTGCCTAGAATAGTGGATCGGCGTTTTGATG |
| | | TAAGTGTAATTGGGTAGAAGGACTTGTTTTACATTTGCGAAATCTTGCTCGGGGAC |
| | | GTTATAATATGGCCTTGAAATGGATGATGACAATATAGTTTTAATGTTATTATAAT |
| | | TAGAGTATCGTATTATTAAAAAGGATGTCCACTGTGGATCCAAGTTAAGCATTAGG |
| | | CGCGTTGAAGAGATTGTACCGCCCGAACCAATGCAATTGACATGCCTAACTAGCAA |
| | | GACAAACGTGTTAAGACTAAAGTCCCTCCTATCAACGTACACCTCATACGCTTGAC |
| | | TAGGTAGAATACTAAAATACTCTCGTAATGAATACCTATTATCTAAGTGACTGCTG |
| | | CGTTCTTTTAGGTGGTGAACTGGCTCCGGAAAGTGTGCTAATAGTCTATATGTCCG |
| | | CGCCTGCCACGTAACCACGAGGCGGATCAGCTAGAAACATAAAGCCGTTTGAGCAA |
| | | TAAGTGACTATACTTAACGGTCTGTAAATTCGCGCTTCAATACCTCTTACTCTCTG |
| | | CGTTCTATCCCGTCTTTTTATAAATTCAACTATACGCTCCATTGGTTATCGCCATA |
| | | TGAGTCCTTATCTACTTAAACTGGCTACCAATTCCTTGCTCTAAGCTAATGAAAGT |
| | | CCATTCGCAGGATTACAACATCAATGCTAACTTTCTCTTGCATACAGTATATCGTC |
| | | TAATAAATGTATAGGCTCCCGGAGGTCGGAACAGCAGTACTCCCGGCCACGTATCC |
| | | CGAATACAAACCTTATTAGTAAAGGAAACACTAGTGAGAGCGTACGGGGATTACTC |
| | | GAAATATCGCAGGAAGGTGGTTAATATGCCAAGGAAATACGAATAATTCTCTCCGC |
| | | ATTCCGAAACTGTTAGCACATAGACAAGACAAAGAGTTTACTGACACATCTTTTGA |
| | | CAACCCGCACTCTACAACGACCTACTCTTTATACAAGTACGGATTATTGTAACGCT |
| | | CCAGCCTAGAGAGAGTAACCCGGAGTTATATGGAGTCGCTTGAGGAGAAATATTAA |
| | | AGCTGAATTCTGTTACGACTAGTAACATTACCAGCCGAGGTCTGAATAACGTGCCT |
| | | ATGGCGATCAGGACAATACGAGAGAATTTCTTCTACCACACTATGTGCAGCAGCTC |
| | | ACTCAAGAGTCCTATGTAGACTGTTTAACCAGTAAGGATTGTTGTGCGGAAGTGTA |
| | | ATATGGTCGAGAAATACCGCTAATATGGATAAGTTAATTGAACTTCGGACGTCACA |
| | | TTCTCCTATAATGAGGATCTATTCAAATCGTTTTGAAGTAACCTCCTCATTTGAGT |
| | | AAACTAGGCTTGCCTGGAGATGGGGCCCCCAACTGTAATGTGTTATGTTTAGTTTG |
| | | AACTCAGTTGGCTCAAAGTATCCCGCAGTACTAATATTAAATCTTGTTATTGTACA |
| | | GCTGGCGAAGAAAGTTAAGAAATGTGACTCCTATACTATTACTGGATTTACAAAGT |
| | | AAGCGTCTTTGACATTAATTATGGTATTGACAAATCAAATGAGAGACAGTAAGATG |
| | | ATGACATTCGCTCATATTGTATGGCTCGTTGACTGATGCAAATAGTACCAAACCCT |
| | | TTTTTTAGAATTCCAGATGAGGAATTAGATTTTTCAGTCAATAGTTACTTGTTATG |
| | | CCACGTAGGCTTATGTCCCCTAAATCGCATATAATAAGATAGAGTGCGAATGCGTG |
| | | CACGTGTACACTAATCAGGGCAAACTAAACATTTAACCTTTGGAGAAATTCCGTGG |
| | | CGCTGAACTTAGTGATGATATATGATTAAGGGATCCGTTTTGTTTTCGATAATCTA |
| | | AGAACTGACGAAGGCACTAATATCGGAGTTACACAGGAAATAGAATGTCGCAAGAT |
| | | GTGCCTTAGGAGTCAGAAATCAACGAGTGTTGATCCCACA |
| 29 | 39.00% | ACAACGACTTTCGAAGGTGGCTGAAGAAAACCACATGATAAAATCGCGAGTATGGT |
| | | AAAATTAGCTACCTGAGTATATTTAATCGAGGTTATATCTTTTGTGAGTCGGACAC |
| | | AAATTCTATATTTGACGGAGCATAGGGCAGACGGACATATAAAATTATAAACAGTC |
| | | TGTACGGCGGGGCCTCCAATTGGATTCCCGCGATCATATCAGTCAGTTGGGAACCA |
| | | TAAATTGCGAAACTCAGTACTATGCTTCAATGCCCCTTTCTAACACGTTTATCGCT |
| | | TCAACCTAACGGTATTTGCACTCCGACTATCGTCTTATGCCTCACAATCAGATGTA |
| | | ATAATGCGGGATTTATAAAGATTTTGAACCATTGGACAACTGACGGCTTCTCATCT |
| | | CACCTTGACGAGAGTATTTCCTATTAACCTGAATTTCGCTAAATACTTATCTTTAT |
| | | CGCCAATAATTCCTTTATGATACACAGGGCTTCTCCAATTCATCCACGCAGAAACT |
| | | GCCCAAATGAGGAGAATAAAAAACTTTATAATTAAATGAATTTTATAGCCTATGCG |
| | | TATCCCCCTACTTCAAATCTGTGCAGTGATGATAAACTATTGTAATGAAGATCATT |
| | | TAATTCGCGAGATTAAACAGATTCATGTTCTAATGCGATTATTCTGGTGTGATATC |
| | | GTGCATGGATAATAGAAAGCTGATCCATTTAGAAACCAAGCTTATGCCTATCCGCA |
| | | CCTTTAACACACGCATAGATTAGCGCTCTGCGCGAATCCTGCGCGTTGCAACTGTA |
| | | CTGATACAATGCGCACCAAAACAACTTATACTCTAGCAATGTACACACATATTGCG |
| | | AGCCAATCTGTTCAGTTTCCCTTTGATATTTCAGGATAATCAGATGGACGCCAAAT |
| | | AGATTACTCTTATACTGAGGAAAATATGAAGTTCAGGTTCAGCGTTACACGCAAAT |
| | | CAGCGATTAGGTCTGCCTAATATGATTTACGTAAATAAATCTACCAACTAGAAATC |
| | | CGGATATTTTACAATAATCATGGCAACGGGTATGACCACTGGGTTCGATCCATATA |
| | | CCTGATGGGCTCGGCAAAAGTCTGTAAGAATTCTCTACATCCCGATCGATGCTTCT |
| | | TTATTTATTTTACTTCATAAACTCGTATTTAAGCTATGCATTGCCAACAGGGCTTA |
| | | AATAAGAAAAAGTGTTGCACACAGAAGTTGCTATGCCGCAATGGAAAGAGTACTTT |
| | | CATGAAAATACGTAGATATTTAGGAGCTTTCATTTAGTAGGTCATCTGGTTGACCA |
| | | TATACTAATCGGATACTTGCGAATTATTGTCCTTTCAGCAGTGAATCCTGAGACTG |
| | | ATAAGCCAGCAGGCGGGAATCGTATTAGTAAAATTTAAGGACATCTGAGTACGGGC |
| | | GAAATCTACAACACGACGAAATCATCAATCTATTATGACATAAGTATTGGACAGTA |
| | | CGTCTGACTGGGAAACATAGCTTTATGTTGGATATGTACATTAGTGCAAATCTGTG |
| | | TTACGTGTTAAATCATCGCGTTCTAGAACTCTTAATCACATAGCGAGCTACCTTGG |
| | | CGAACACTCGTTACTGTTCTCGTTTTGCTATCATGTCCTAAAAGCGGCAAAAGTTA |
| | | TTACTGCAGGACCGAAAAATATGAAAAACTTATTTTTTCATGGGACTACACAAATC |
| | | GAGTTGAGCCTTTAAGCGGTTCTATGTTACTTGAGTATCTTGAACTTGGAGGGGGG |
| | | TTATAATGATAATAGCAATACATAGGTTATGATAAACTGTCCTGTTTTAGATACAC |
| | | GGGAGCCTTAGTAGGCTTATTTTAATAGTGTAGTTGTTGATATGAATAATATAGAA |
| | | AGGCCATGGAGGAGAAGTGCTATGTTAAGAGGGCAGTCGCGGTCACGTGTGCCATT |
| | | GACGCTCACTTATATGCTGCGTTTTCGCAGTGTCTCAAAGATTAAATTAGCCATAT |
| | | GGTGTCTATTGTTTTCGTAAACGCCTAGCATGCGTTCGTC |
| 30 | 38.90% | CTTGTGCGTCGAAATCGAAACTCAAATAGTATGTACGCTGAAAATAATAAAGCCTA |
| | | GCTAACAATCCATCCGCGTTTAGATCGTAATTCACATTTTACCGATAAAAAGTTAA |
| | | GTACAACATTGGAATTGTTATTACTTAGCCAGCCAATAACGCGTCCTAATTACCAA |
| | | AAAAAACAGACTCTGAATCATGGTAGATTAATTGGGTATCGATAACATTATCCAAA |
| | | TTCAGGGGGCCATTCGCTTAAGAAAAGAGATGTTAACGTACTCCAGCGATCTGCGG |
| | | TGTTCTGACTGTAAAAATACGCATACATTTCACCCATAGCAGAAGACGTAGGACGT |
| | | CTTTTCTACCAGGTGTCTGTATTACATACCCCATGCATATCTAAAAGGATTCTGGA |
| | | CGTATTTTGATTTTTACCAGTTGAGATAGTGTCAAATTCTGACTTTCAAATGACAA |
| | | TCGCAAAAATGTATGCGAAGGCTGATGATCTTGTAATCAATACTGGTGCTAGTCAC |
| | | ATACTGTTGTAGATACGCCAGATTTACACTATACACAGTGAACAAGGTCATGTCAA |
| | | TAACAACTATTTTTGTTTATAATCACTAACCCTGCATATGAGGGTCTTGATCCAAG |
| | | TTCGAATGGTTGAGAATTCCGAGTTTATTGGTAAGGGAAGATGTATCAAATATAAT |
| | | CCTTGCTTACTTCCCAACAGTCACAAGAAGCAGAGTTAACGACTGATTACGGCTGG |
| | | ACCAATAAATATTGAAACATCGCAATAAAACTTGAAGAAATTTGACTACAAAGTTT |
| | | AAGTGTATACAGTAGATCGGTTAGGGTATACTCAATTAGGGCGGAACCCGCATTCC |
| | | TGTCGATAAGCTAGTAGTAGGTGGTTTTCAGGTTGGTATCAACCATCAATATTCGA |
| | | CATACATTAATCCAGTGAATAGGGGCGTCCGGATTTTGTAAAGCATTAACCTTCTG |
| | | TATAAATACTGCCAATCATATGGCTTGAGTAACCGTTTTTGTCAGTGGAATCGTCC |
| | | CCTCGCTAGAAGCATCTGTACGATATCTAATGGCTGTAGTTGCCTTAAATCGGAAA |
| | | GGTAAGTCGGAACCTGGGCTCTCATTCGAATAAGACCAATCCTAAACGGCGAATTC |
| | | CTTTATCTTGTTAACTGCTGTGTCAAGTCCTCTTATCGAAAATTCTTACATGTTTA |
| | | CTCTTGCGATTAACTATGGTGAACTAATCCCAACAATGACTGTTCGTAATAGATGT |
| | | GTTTGTAAAATTAGTATTTTGGTGACATCTCTAGTCATTTCATGCCTTCATAGATC |
| | | ATCGGTATTTCGCAATAATCTGCTCATACTATGTACAGAAATACCACTACCTTCTG |
| | | ACACCCTTGCTAGCACTCTGGAACTAAATAACTCATAGACGAAAATACAATGCAAA |
| | | GCTCATCTTCTTTTGAATATTGAGCGAAGTAGATTGTTGACGTTAAGAAATGAGTA |
| | | GTTTCATTCGAGAACATCCGTAATCAACTACAATTATAATCTCACAAGATCGGTCT |
| | | ATTAAATCGCTCATACTCCTAGGACTAGAACCAACGATCGAATTTGTGCTTTGGGC |
| | | TTAGGTAAAGACGTATAATCCTACCTAGAAGTTATCCATTTATCCACTTGATAACA |
| | | TATGTCTATTCCCCAATCATAATAAGACGTAGAAGAAAACGACTCTCACAACGACA |
| | | GTATGCCCTAATATGCGATGGCGACTGAAAATCTTACGGCGCCCGCCTCAATCACG |
| | | TTCACGTGACCCAGCACATTAGATCCAGGACTGACTCAAGATCATTACTCGGCGAT |
| | | CAACGCACTATCCTCAATTGGCTATGTGCGAACTCCTCGTATAGGATAAGGATATT |
| | | CCGGTCTCCGTATACGCTAGGCTCAGTAACGCGTCTTACTCTGGGTCAAGGGTTTA |
| | | AAGATCATAGCGGTATCATACAAAAAATCATATGGCCTACTTTGTCGTTTTAAGCG |
| | | AAGATCAACGACGTAATAGCTAACTTAATGAGCAAGATTT |
| 31 | 40.20% | TCGATAGGACAGATAAGTGACCGCTTGTTGAGTCTTATATGTATTGGACTTAACAT |
| | | CGAGCAACAGTCTGTAACATATGTCACTACGTGATTGAAGGCCGTCGTCAGTAATT |
| | | AAGGATAAGGCGGTAAGACATAAGATACCGTACAAGGATATTTATCGTTATCTCAA |
| | | GGTCAAATCTAACTATAGGTAACAATTACCTTCTACTAGTAGGGGAATTCCGTTGG |
| | | ATAGCTAGTAAAAGATTGCTTCAACTAATCCAACAAAGTATTACATCAAAACAGAT |
| | | TGGTTATCAAGATTGGAGCTTCAGAACTAGAGTGGTGAGCAAAGCACTCTCATGCC |
| | | TTTTGTAAGAACCGGGAATGAACCGCAAGAATCACTTGACAAAGGTATTGGGTGGT |
| | | TATGTTGCCGGGAAGCTACGATTATATCCAATAGGCTACGGTCGTTGTACAACCGG |
| | | TTGTCTATCTGGTACTTGGTTGATGACCTAGGTGCGAGCCATTCTGCCAAATTTAT |
| | | ATGGAGATTAAGAGTGGTCTTTGCCTGATGAAAGGGCCAACTGCCGAAGTACTTTG |
| | | GAGCAGTGTTGACTGCAGCTCCAAACATCTTGTATTTTAATATTTCGGAATAGACA |
| | | TCTATCGTTAGTGAGGAAAGAATTTGATCCCGCGCTATTTTCCCGACATTCTCAAC |
| | | ACTTGGATTACTTAACTCATAGAATTTTCTACCTATTATATTATAACAAAAAGGTC |
| | | AGTATTGGTCCTGACGTATCTGATTCACGTATTACGGGGCGGGGTGGAAAAACTTG |
| | | GTTTCCTAGAGCCTTAGACGAGCGTTAATATACAACAAACTAGTTTCACATAATAT |
| | | TACGTATGGAGTAGACTCAAACAATGGATCGCGGCGACGTGGATGGTATTATCGCA |
| | | TGATGCAATTCTAACGATGAATTTGTGTCCGCGCTGTTGTCGTTTTAACAACGATT |
| | | TTGAGGTTATGATAGTTATAATCATTAGAACATGTCCGAAATTCAAGTGGTTCACC |
| | | TTAGCTTTGTCAATTTTGTCACACTTCAGGGAGGGTCCAGGAGGAACTGCAATCGT |
| | | CAGTCTGAATCGTTCGAGCAGTAGAAATGACCTAATTTGCTCGTGACGTACTGACG |
| | | ATACCAAATCAATGATTGAGTTCGAGGATCTGATGTTTGGAGCTTGCGTTGGACGA |
| | | TCTGATACTCAAAAGTCGACACTCAACATTTTTTGCCACGACAGATATTCTCCAGA |
| | | CTTAAGAAATCCTTGCTGAATATCAAACATGCAGCTTAGATTAGTTATTATGTAAA |
| | | TTGTGAGATACTATGCTAACTCGATAGTGAGGTGTTGGTCTGACACCGTGAATTAA |
| | | TAGGTCGTCCTTAACAAGTACCACTTAGATTCCTCGCTTTTGAGTCTTTGACGCCT |
| | | TTGGCCGGATGCATGTATAAATCCTTTTCAAAAGGCTGTTCATTCCCATCCAAGTT |
| | | CTGTAATAGGTCTATCTTTACTTCTGGTAACAAGAGGGAGTTGGGTTACGACGAGT |
| | | AATTGTTGTAGCAAGGATAAACTGCTATTTTTGATTAACAGCCTCACATATAATAC |
| | | GGGCAGCCAAGTCAGCCTGCCGGCAAATTTAGCAGTGTTTCTGCTCGCCAATGTCT |
| | | CGAGACTCCTAGCTCTCTCGTCCATTGCTGACTAGAACTAGCCAATTCGGCGAGCA |
| | | TTAGAGTGCTAAAAAAATCGGTACAGGAGCCTAAGGGTATCCGGGCAGAAGCAAGT |
| | | GGTGCCAAAGACAGTTAGTTTATGAGCTTACGTCCAATGATAGAATTTGCAAACGG |
| | | TATGGTTACCTTCTTTTCTGTATCTTCTCAATGTAATATGTTAATGAACACATTGT |
| | | TAATGTGGTTTCATATAGTAAAGTAGAAAACTAGCCGACAACCAAAGTAAGAGGAG |
| | | CAGTTTTAGAATCAAATACACCAACTTAAAAATTTGCATCTATGTTTTTGACAATT |
| | | GACATACGACATAATAAAAGTAGGATAGTTGTAGATCGTC |
| 32 | 39.90% | ACAACAATCCAGAATTAAAGAGTCAATGATTAAAGTCTCTATAATTCTTGGTGGTT |
| | | AAGGTGCAACTTTTGTCAAGCCAATGCTTCTCTAGCTTACGAAAGGAACTAGTATT |
| | | ACAATTTGTTACCGCATATACTAATGATCAAACATTGTACAGGTACGGTTAATAGG |
| | | CGCACTAGTAACACCGTCAATTATTATCCTCGTCCGACCTGAGAAAGGATGATAGA |
| | | TCGTGCATAGAGGGACTTGTGGAACGAAGAACATTTCCTACGCAGCTACAAAAGAT |
| | | ATATTGCACCAGGGACGTCACACTAAAGATGTATACTACAGCATTGTTTCTCATAA |
| | | CCTCTAGGTAGGTCTGTAGATTCAGCGTATATCGACTACCTACATCTCGTCTGATA |
| | | TTCATCTATCGCCTTAAAATTGTGTAAAATAATCTGAGGTCATCAATGGTTTTGTT |
| | | TTTACATTATGTAAGGTCCGTAATGGTAACTTGTGAACCGACATAGTTCCCCGTCG |
| | | CTTAGGTGTGCAGATAATTAGATCCAATGGATCAATTCTCGGAGATAGTCTTCTAC |
| | | GGCATTCTATCTGTACACGTATTGGTACGGGGGTCGTAGGCAGGGAGACATCTACA |
| | | AAAGTTAGCGGTTGCTGAATTATTAATATACAGCTTTACGCTTATACGGTTGACTA |
| | | CAAAAAAATTACAAGATTCTTCATGAGATTGTACCTGTCAACTTAATTCGTATCAA |
| | | AAATTCTAAAGTGCGCATCTAACTTCATACAACGGAGAAAAGTACATATAAGTAGG |
| | | GTGTGAACGCAGATAACGTTCAAAATGATTTAAACTATGATTGAGATGTCCAAGTT |
| | | AAGGACGGTAGGGTTGCTACCGTGGACTATAAACCCTAATGCCTAAATCTTTATAT |
| | | TCGGGAATTGTTTCGGGTTAGGGGGAATACGCACGAGGCTAACACAATATGCATAG |
| | | TGCGTATCATTAGCGTATGGAGGACGAAAAGAGATATACCCAATTATAGCCTGAAT |
| | | GTCTTAATCAGACCCTTATCGTCATCTCATTTTTGACTACAATCGGTAATAACTAC |
| | | TCGGGTTTACTAGATCCTAACGGGATGACTCATAATAGAACGAATAGTGTAAAAGC |
| | | AACCTACGCGTAAGACCTTCCCGGTCATGAGGATGTCATCCTATGCAAGCGTTCCT |
| | | CCCGCGAACGCCACGTGATCTCTCGATTCCATTCTATAGGATTCATTAAAGCTCTA |
| | | CTATTACCCCAATTGCTGGGTGTTCTAAGATCTATAATGTTATTGTCCAGATTAAG |
| | | TTCTCCTGCACTACTCGCGATTGTGTCTTTCGCCCGCTTGTCCCCCCGTAATTGGA |
| | | TCGGGCCTTCGCGTTCTGCTAATATTTGTTACGTCACGTCGGATAACCCCTACTTG |
| | | TGCAACATCCTGACGAATGTTGTAAAAAGTTTTTCTTTGGAAATTTGTACAGTTAA |
| | | AAGACAAGATAATATGATTGGATGGCAAGTGACTGTAAAGTTCTATCCAGTGTTTC |
| | | GTATACGATTAATGAAACTAAACGAGAAACTTTGCTGACCTCCACCCAAGATAGCC |
| | | TTCACTCTTTCACTAACTCCACGGTGAATTTTTTTTAGTAATTTTCATAAAGGCAA |
| | | AGACTAAGTTTACCTAGTAACGCCAATCCCCCCACCATAGTACACTGTGATTCGAA |
| | | AAAAGGATATTTTTGAGCTTCTATGCTTTAGGGATATTTAGTTTAACGGAAAGCAC |
| | | CGTCAGCTTGGAATATTAAACACGCACATGATTTATGGACCCATAGTTGACATCAA |
| | | GGTCTTTGATACCGACGGTTTTCGTATTTTCCAGTGAAAGCCGAAGCTTTACAAAG |
| | | GAGAGAGTAATTGAGCAAATTTCTCACTGCATGTCACAGGGACTGATAAATTAGTC |
| | | CAAAAACTTTATTACGTTTGACCTTAGAGGTACCCTAATGCGGCTTATTATTTGGA |
| | | GGCCAGACTATTGCGCGTAACAGGCTGTTTGAGCATCGGT |
| 33 | 38.20% | CTCCTCGAGCTTATAGAAAAGTCAACGAATGTGTAGAACCAAGAAAGTGACCAGCT |
| | | ATCAAATAAATAACAAGTGAGAGGTACAGCGTATCTAATAGGCGAAAGTCTAGCTC |
| | | CAGGTATCGGTGAAGTCTAACTATGAATTAAACGCATTGCGTAGCTACATGGTTTT |
| | | ACACGCACCATTAACAGGCGCATAACTACTGCCTGAATCGCTCTGATATTAAAGTC |
| | | AAAGGAAGCTAAAGACTTGCTATATCGTTGCATGGTGTTAAGTAAATACGACTCGA |
| | | GTATTTTAAAAAATCCTCTGAATCGACCAACTATTTATTCGTTCATTCTCTGTCAT |
| | | TGAGTAGCGCTAATCAATGTAGTATTTGGATCAATAACCCTCTGGGTTAGGCGACT |
| | | ACATGAGTACCCTTGGAAAAACTCTGGTCGAGCAAAACAAGACACATGGGGTTAAA |
| | | TAAAGTCTATACAGTTTATAATTATGCAAATTTGACGAATTTTGTACAGAATTTTA |
| | | TCTATAATCTTACGGGGGTATACATATGACAGCTTTCCGGTGTTACAATACTCCTT |
| | | GTGCTTTGTACACTTGGCGGAAAATTCACCACAATGTATGGGGTTCCGCGCAAGCT |
| | | CTCTTTTTCGGTAATCTGGGATTCCTTTTTTGTGCCCTTTTACATAACAAGACGAA |
| | | TTGGTCTCCTTTTTACTCAGAAAGAATTATAATACTTTTCTTACTTGTCCGTTTCC |
| | | CCTCATCTTTTTTTACCTCCAAATCCGATTCATCGCCTTAAGTCCAGTGTCTTCCA |
| | | ATGTAGTGGTTTAACGCGAGCTACATAACCATCCCGGATGTATACGATTCTACAGC |
| | | GTCTTGAAAATATTATGTTTAGGTTTCGGGTGAAACGCACCTAGAAATTATAGCAA |
| | | TAATAATCTTAAATCTCCTCATCATAATAGATAGGTTATTGATAGGCGACATGAAA |
| | | CCCAGCGGATTCACCTATCACCAATCAAACCACAGTTCCTTTTGATGCAGTCATTC |
| | | CTACAGGCATCCTATTAACAAACAAGCGTGTGCCGATGAAGAATTCGTATCTGTTA |
| | | AGCATCCGACGGCACATGTGCAAGAGTCGATCTCCTGATACCAATTTTAGTACTTC |
| | | TCCTCTGATTAAAACAACTTCCAAAGTTCCAACAGATGGAGTATAGATAATCAAGT |
| | | TTCCAGAATTAATCAGTAATTTGACAAGTGGAAGCGCTAGAGGACTATTCCCGGTA |
| | | ATACTATAACAAGTAATAGTGACCTTGTGTATAAATAGACGTTGATAGATATATAT |
| | | ACACTTCTTGATAGCTGAGGTAGACGTTGATACAACCCGCAAGTGAGTCCATTACC |
| | | TTAGGCCCTACGAACATGCTCAAACCCTTTTATGCTTTCCCAGACTCAAAATCAAT |
| | | ACGTAGATATATTGTAACCGTATAGAAAAGAGCTTCTGTTGGATACAGTGGTATAA |
| | | CAGCTCATGTTCAAGGTTTATACGGTATGACAAATGTGATTTTCTTTTATGTGAGA |
| | | TAACCGAACCAATTTCGAAAGATTACTACTAGTTGAAATACCAATTTTAAAGGTAT |
| | | CCTTTCCATTAGACCCCTTATATTATTCTACTGTATTAGCAAATTTTAGAAAGTTC |
| | | GTGTGGTACTCAAATCCGATGAAACTATTCACCGTGACCATTAAATAAGTTTGATG |
| | | ATCACCGAGAATTCACACCTCGTAAATAACACCTATCTTAATAGAATTCGTGCGCA |
| | | GCTCTAAGAGAGAGCATCTTCCAAAACGAAGAGCTGTTTACAATTGCTGCCACGTC |
| | | TTTGATATACACTCTTTTATTGTCCAATCCGATGTTTCACAATAGGATCCATGGTT |
| | | CCGGTTACTTCCTAGCTAAAAGGGTTTGCCCACGCGGTGAGGGAAGTCTGTCGGTA |
| | | TATTAGACGTAGTGTTCACGAATAAGTAAGATTTTTAATTTGGAATGGTTTGCAAC |
| | | AATTACATAAGGATAAGTAAACGCGCCGTATAATGCTCTA |
| 34 | 40.00% | ATCTCATAGATAACTCTATGAGGAGTTAACGCCTAGAAATTTTGGTCTGCATGGTA |
| | | CAGTTACATATCGTATGAATTCGTCTAACATTTGAACGGACCACACCATCTGATCC |
| | | GCACTCAATGGACAGTAGGCATTCGGTTACACTTTCGTCTGGAAGAACAGTCCGAA |
| | | TATGAAAATATGCTTAGATGATTCCAAGTTAATTTCGTCTATAAATAAGTAGCTTT |
| | | TGCTCTATAAAGATAACCTCCTACAGTCGTAACAGAGCTCATATACGATAAGAAGA |
| | | GTATACTTTTAGTTTTTCGCACATTTAGCCATTCAATCGAGAACATAGACGCCTCG |
| | | AGCCGAATTGCTTAGCACATTTTCCTAATAAATGTATTCGAATATCCAAAATGAAC |
| | | TTGCATGACTCCGTAGCACGCACTAGATTTAGTGTGCCTAAAGATTAATATCCCAA |
| | | GGTTGGGCTAGAACTAAAAACGCTGTTGCCAATAGGTTAGATTGTAAACTGGCCCT |
| | | TAACAAGCTGATTATCAGGTGCTTTGGATACTTAGCACATACTTAACACATCGGCG |
| | | TGAATAAGTGGGAAAATGTGCACAAACTCATTAGAAATTCTGTGATTGGGTCTTTA |
| | | CGTTATGTTAAAGTTGGTATTGCTTATAATAACTTATTCTCGCAGCGTACTCGAGA |
| | | ACGTTTGAATTCGTGAGAGCCCTTAAATCAACGACCCCCGGCGTTTAGAAACGGCA |
| | | ATCCATATACCTGTCATAAATTATCTTAGAATTATTATTATACCCTAGCCTTAGCC |
| | | ATTTTGTTTACCAGAACACGGATGGATCTAGTTACGATTCATATAAAGTGAGAGAG |
| | | GCTAGTGTTGTAAGGGAGTGAGAGAGCTTGCATCTTACGAGCTCTTAGCTCCTCTT |
| | | ATCAAAATATCATTTGGGCCCAACAACGCGTAAGTCAGATGATCTATTAGCAGTTT |
| | | GGATATGTTCAAGAAGTCCTCCAGCGGGTTTGCGAGATTCTCTGTATCGTTGACTT |
| | | GTGACATATGATTTGTATTCCAAGACGGTCAGTTGCAATCTTGCCTGAACTAGTTG |
| | | GATTATCAGCCACCCCAGGCTGTTGCATCTAATTAAGTTTTCCTATCTGTAAAACC |
| | | TTTCACTTAGCAATGGCTTAATGCTCTTACCGATCAGCTGGAAGCCGGTAGTACTG |
| | | TCACTTGGTTTTCTTAACCTATCAAAACGGAAACAAGCCGTATTTTTGATGGTAGC |
| | | ACTTCAAATGGTGGGCAACCGACTAAAGAACGTCACTCTTTAAATTCTCATAAGTT |
| | | AAAATCGGATGTCGAGTCAATATTTTGTCGGGCCATGGGAAAGAGAGCAGTATGCT |
| | | ACCTTCTTAATCTCTACCTTACTTTAGACAAGCATACGTCAACAACTGTGACTCTT |
| | | CAAGGACGGGTATTCCCTGACTCAATGCTTTGGAAGAACATTTAACTGGGTTCCAT |
| | | TATAGTGGTCGGACTCTTTATGCTTATGTCGCACCAGGTCCATCTATCGAATTCCT |
| | | GTATTCTATAAACACCGGCTGCACTCTAAGAAAGATCGAGCTTCTGATTCCAAAAG |
| | | TCTATAAATGATCAGTTAGCCTAGCGCCGACACATTGCTCCGTTAGAAGCTTGACG |
| | | TTTGTTATTATGAGGGATCACAGATTACCGTGTGTCGATTGGTGGCTCACTTATCT |
| | | ATGAGCCAGTTTCGTTATGGTCATACCTTTAATTAAGGGAACATCGTGCTAAAATT |
| | | TTTAGAATGGGGTACTGTCTAGACTGTCTCGAGGATTCATGCCGATGAAGACCTGA |
| | | AATTTGAATCGGAACTTTTGTGGCACCGCCGTATCGCAAAATGAGAAAAAGATATC |
| | | GTTAACCCCTTATAAACCGCAACTAACTAAGTCAAAATAAGTCGACGTGACTTAAG |
| | | ATACTGATTAAGAAATGGTATCACGGCTCTTTTGCAATACCATTACCAAAATTGCC |
| | | AATGAAACTGTTTTGGCCTATCTTAAGCCACGAATAATAT |
| 35 | 40.40% | ATTCTTAAAGTCGATTCGGTGTCATAATAGGGTTATCTAACATATGTACAAACGCC |
| | | CTATAAAGTTATTATCGGACTGGTGCATAAGTAACAGTTCGCTATAAAGTTAAATG |
| | | CTATCAAGAGAAATAAGGCATACTGTGATGAAAACGAGGTCGTACAGAAACACCTG |
| | | CAGGAATTAATCTGCCGTATCATACAAGGAATATCGTTGGAGTCAAGATGACTGCC |
| | | CATTTGCAGTTGTCATCTTAACTGATGATGGTTTCTTGCTTGATAGCACCCGCCTC |
| | | AGTAAAAACAGATGGAACACTCCAATGCTAGCCAACTGAAATTTAACGTTAGTACC |
| | | AAAGGCATCCAAGCAGTCCCCTGGCTAAGTTGGAGTGTGGCATCGATATAAAATAG |
| | | TTAAAAAAACGGTCTGATGTTTCATGCAGTCGCAACCACGCATACGGTTCCGGTTC |
| | | GCAACGATTGATGTGGCGGTCTCAGTATTTTACAAGTTTTAACATGTCGGCAGCCG |
| | | CTAGGTAGATACCTGCACCCTGTGGTTTCGTATATAGGGAATTTCGGTGCTTTAAG |
| | | ATAAGGATTACTCATAGGGGATATTACTCGATTGCCTCGAAAAATGCGATGAGTCT |
| | | CTATATTCAACGGTCTATTACAGGCTTTCTATTTTCTCGGGACGCCTAGGAGTTGA |
| | | ATGATGCACATCATTAAGCTACTTATGCGGTCTTCCATACCATTCCAATGTCGTCG |
| | | AAAGAGGATGCAGTGACAACTCAGGATACTAATAATTCCTTGAGAACTGTCTATTT |
| | | CAAGCCTATTCTAACATAATTAGTTGCTAGCCATATAAGAAAATATCATCAAACAG |
| | | ATAGGGTTGATAACAGAGGGTGCTGCCCGTATAGTGAACATCGTAACCGGGTTTCA |
| | | CATCCTAGATTGGTGGCCTCCTACTATGTAAGATGTAGTTATACTGAATGTGGTGT |
| | | TGTGATCAAGACGTAGGAAAATTTATCAGATATGCCAACTAGTATCATCCTGAGTT |
| | | ATAAAGGGGGTAATTTCGGACAAAGGTGTTGTTTCAAAAGGTTCAAGCCGACGTAC |
| | | CCGCACATCAACTTATCTTGTAATGATTCAAGGTTTATGTAGCTTGATCACCAAGC |
| | | AACCCAAGCGAGCTGTACCAGATACGATTATGTTAATAAAGGTTTGGCGTACTAGA |
| | | CTTAACGCTAAGGTTTCGTAATGTAACGCCTGCATTCACGTCAATAATAGCTCAGT |
| | | ATGTGAGAAGTCCGATGCTGTTAATTCTAATAACGCTCCCACTTGAAGGAGAAAGC |
| | | GGGAGTAGGTGCGTTTGTTCAGAAACCACTTAAGCGGTTTGTTTGTACGTACAAAA |
| | | TTTGCTTTTAGATGTATAGTTGTATACATAACCATCGTCCGAAAGTAACCTTCATA |
| | | TGAAACTCAAAGGCATTAGTTGGGAAGCAGTATGTGGCGTTTGTGACACATCGGGA |
| | | TTATAAAATTCCAATATATATTCTAAGTAGCAGTTAAATGAACTCCACTATGGTTA |
| | | AATACTTGTACCTATCGTTATTCGCAATTGTGCCACTTTTACATAGATTGTGAACC |
| | | GGTATATCGCGTGGTCAAGACCAGGCTTCAAAGCTGTAGAGAACTGTTTATTCTTT |
| | | GAGTGACATAGTATCGAGACTTGTATAAACATGGATGGTACACAACGTTGGAAAAG |
| | | CCGAAAGCCAATAAGATATTTAAGCATTATGCTTTTATGTCAACACTGACTTTCTA |
| | | AACCACACACCTTAAATCAGTAGAACAGCATTTTGAAGGAGTGGCTAAACCATGTT |
| | | GCGTGCAATTCTCCGGGCTCGTAAAAACGTGTCGTGCTAAAGGCTCTAAATCTCGC |
| | | AGTAAAGGAGGCCCTCCAAACTAACTTAACTCATTTTGACGAACTCAAGTAGCTTC |
| | | TATTAAATTCGTCCGAATACCATGAAGAACGGGATTCGCATACTGCGTTCGCCGTA |
| | | GTGGAGCTCGTTACAAATCAAATGGATCGATAAACAAACG |
| 36 | 42.30% | TTAGTATAGTTAAGATAATGCGTCGCTAAACAACATAAAGATTCTTTACCGATGAG |
| | | TTCTCGCTGGTATTCGCTTTTTTAGTCTTACTCGCTCAAGTTATCTTGAGAGATGT |
| | | GGAACTGAACCACTTGAGGTAGCCCCATCAATTATAAGGAAATTGAAATAGGATCG |
| | | AAATATTCTGAACTATTTCCATCTAGTCTACTGAAATTAACATTGACACCTTTCAC |
| | | AAACGAATGGCAAAAAAGGACGGATCCATCCCCACAGACAACTTCGTTTATTTCAG |
| | | CACATTTGTCCCTGGACAACAGCCGTATGTGGTTCGACATACTACCTGATAGTGAG |
| | | CGGTTATCGAAATGTCCTTGACTAGCTACTAAGAGGCTTTATACAATATTCCTACA |
| | | CACATAGACCCAGTAGATATGAGTTCTAGTTGGAGATTTTTCAACACAATTACGCC |
| | | ACGAGGTCCGACAACGTATCCTCCACAGTTAGGAACATTTATTACAAGGAGGTTAG |
| | | CTCCGTGCTACAGCAACACGAATTACTCCACCGTGTTGAGCAGGTAAACGAGGGCA |
| | | AAATACACCCCAAAGCGTAACTGCATACGACTTTCCGCTCGAAGATTGTTAAAACA |
| | | AGACTGCAATTTCTGTGGCAAAAGACACTAAAGATGACAGTACAGCACCCATGGAG |
| | | AGTTTGTACCCGGTTCGACCTAAGTATCTGTTGTCCAGAATCGTGAAATTTGAAGT |
| | | GGCCTAAAAGCTGAGACGAGTATAGTAGGGTGGAGGTTTCCTATATGTTGGTCGGT |
| | | CAGTAAATATTTAAACCACGGGAGTTAAACTTATCTTAAATGTATCTATACATTAG |
| | | TATATAGGCTGAGATTCGATATATATAGACGCCACCCCGAGAAATAGAAAGATAGT |
| | | GATTCAAATTCCTAACAGTTCGGAGTGGTATACGCATTTCTGAGTAATTTGGCGTA |
| | | CAAAGTTTGAGTAGAGCACAGAGTTGATAACTAGAGCAATGTCTGAGAGTGGATTA |
| | | ACTTGGTGTGCTCTGCTAGAAATCCCCAGTGATGATCTCTCATAAAAAGTGACTGC |
| | | AAGACTAGGATACAATTTATTATCGAAGTATCAAGATCGTGGGTTCCTTTTTTCCT |
| | | GGTCAAAGATGAATCTGTCTTACTTAACGAAACACAGGAACTTTTCTTGCATAGGC |
| | | ACCGATCTTGCTATGTATTGAAGCTACTTCAAAGGACCTATCAGCGGGTGTACACA |
| | | ATGTCGGAACATGCATAAATGGCAGAAGGCGATGAGTCATTTCGCACACCAACAGG |
| | | CCGACGAGCGTAGGAGCGACTCAGAACACTACCAACTATAGCATAACGATAAACGG |
| | | AGAACGTCCATGCCGTTATGTGACCATTCGGTTCGGAGTCGTGGGTTACCGACCAC |
| | | GATAGAACATGGCACACTGCTTTCTCACTTCCCCAATAAGAAACACCCTGGACGTA |
| | | TACCTCGATTGGATCTGGAGACAGTACTCGGATCCACACCTAAGTAGTACCTCACT |
| | | GTGGGCGATGGCCAAGACGCGAGGTTGACTATCTGCGTGGTGGAAAAGGCCGACAG |
| | | ATCTTTATCAATTGTAGTGAGCTGATGAGTCCTTTATCCGTTATAAGCTACTTTTA |
| | | TTGGGTAATAGATGGTGCTCTTACTCCTTCGAGTTAATATATAGAAATCACCGCAA |
| | | AGTTAAACGCAACATGAGTGGTTTGGATTAACAACTTCTGGAATCATTATAACCTT |
| | | AGGAGCGTTCTAGTGATGCTGAAATTGAGACAGTAAAAAGTGCCCATGATGTAGGA |
| | | AAGTCACTATAAAGTGAATCTCTTGTCCTTAAACATAAAGCGCGGTAAACACTCAC |
| | | GTTAAGATGGTTGTGGCCACAACATGACTCTTGTGGTTCTTGACGTGTTAACGCGG |
| | | TGGCACTAGCAGGGATGATACAAGTTGATGCTTACCCATATGATTATTGTTCCCCG |
| | | GAGCCACCACTAAGCCACTAAATGAAGATTTTTGCGGCGA |
| 37 | 38.20% | GATGTTCTGAAGTTCCTTAGCGTACAAACACAAAACGTGCATTGGAAAATGGAGAG |
| | | GGAACCCTCTATGTCTGATGATTTTTTCGGTTGAGCTAATTCCAGTGCAATCGACA |
| | | ATAAGGGCATGTCCGAAATTCGCTTTTTAATGGTAGTAGGTCCGGCATCATTATGT |
| | | TGTCGGCCTAAATACCATAATCATTGCTCAACCTTCAACTCTTTGCTGGAACAATT |
| | | AGTACTTTTCGTTTGCGCTTAACCATGCGTATAATGTAATAAAAGCACCAGTTTAT |
| | | AGATATCGGAAAATTTAGAGTTCATGCCATAGTTTGAACCGACGGTAGGTACCTAT |
| | | AACGTCTTTTGATTTCCGCAACCTATGTATTGTAAGCAGTTGTCCTAAGGAGTATT |
| | | TTCACTGTCTAAGTGGTAACCAGCGGCGAGAACATAGTCGGCGGAACGGTTCTGAT |
| | | TTCGACTAGCATCGGCGACATTGCCTTGTCAATCTCCATAATGATATAAACATGGT |
| | | CTTTTAACTCTCACAACCTAAATTATTAACAGGTCGATACTTCTCTGGCGAGGTTG |
| | | TTTTAAAACTTCCACTCCGGATAGGAATTTCATTGAAAATATAAAAGGTTGATGTG |
| | | TCAATCGAAGTCTAAAAAGAATGAAGATTAGTGTCGCCTAGGACATCTATTTGTTT |
| | | TAAAGTGCAAGGAACGTGTTCACGTAGAATTGTGAAATTGGATACATGTTTAGTGT |
| | | CATGCATTGTTTATGGGATTGACTATAACTTAGATAGAGAACTAGTTACCCTTATT |
| | | ACTTTGCAGTATATGAACGACTGATTGTCAAGACTGAGCCTAAATTAAAGTAATCA |
| | | GCACATTTTGGATATGGATAGGAGCTCAGTTTCTGGTTTCACTCTCATCGACTTCT |
| | | TTGTCCAAATACGGCAATCACGTAATGCATAAAAATTCAAACATAATGTGATGAAA |
| | | GAACATATCACCCGTCTAAAAAATTAAATATATACTATAGTGCTGCAATACATCCT |
| | | TAAATTGTCCTATATTGGTAAGTCAAACGATACAACCTGCATTCTTGGGGGATAAC |
| | | TGATGTTTACTGGACGGCGGAAATACTTTAATTTATAGGCTACTCCAGTGCATAGT |
| | | AAGAATCATAATTTGGTAGCGCCTAGTAAAAAGAAATCCTCAAAAACTAAACGCTA |
| | | TTCTGATCGCTATCATCAAGAAATGAATTGTAAGTGAGGGCTGTATTCTAACTCAT |
| | | CCTAGCAGGATTTATTGCCTGCATCATCGACATTCTGTTCGAAGCGGTGATCCCCA |
| | | TTTGGACAAATTCAAGGTTTGGATTATCTAGCGCCCTTGGAGTCTCTTTACGTGTT |
| | | TAGGTGTTCCTGTAGGAAAATCATCTTATTGTCGCGAATAGAAGGTACAAAAAGAC |
| | | CTCAAAGTTACCATATGCACCATGGAGATGAAACGGTAAAAGTAACTGGGACCAAA |
| | | GCTGTCCTTCCGGGATTCATTATTACCATAATCATTAGGCATCAATAATATTCTGT |
| | | GCGATATGTTGCTCGGCTTATTAACCTCAATGAAACAATATGACCGCATATCGCTA |
| | | CAGTAAATCTACGACGTTTTTACTGATTGATTGAATCGCACTTTTTAATAATTGTA |
| | | TGCCCCGATACATAAAATGTCATAATCGAGAAGCATATAGTAGTATTGTAGTATCC |
| | | TCAGGATCGGTTGGTAGCTTTAATACGTGTAAATTTTTCTCGTAATTATCGAGAGT |
| | | GTGGAGACGTCCGTGTACTGGATTCGTAAGAATTCAATACCCTGATGTCCGTCCGA |
| | | GTAGATCGATAAAGTAAGTAGGGATATTCAGATATTTAATGTATTTCCTGTACACT |
| | | GTGACATCTCTGCAACGAGATTGTTATACTGGCGGCGCGTAGGAAAAATTCAACCA |
| | | GTCTGTTTGCAGGGATAGTTAAAATTCATTAGAGACCAGAGCAAATAATGAGCATC |
| | | CGAAATGTATCCAAAGCGATATACGCGCTTACAAACTCTG |
| 38 | 39.70% | TTGATGTGCGAATATAACATTGATCATCAGAGGCAAGGTGATAGGTATTAAAACGT |
| | | TAGCGTCCACGCTCCTGGTTCTATAAAACTTCTTTAGATGCTGCTAAGTCCATTGA |
| | | TTTACTGTTTTATAGATACGAGAGTAAATATAGTTTAAATTTTTTAAGTTTGAAAT |
| | | ACGTGTAGCTATCGTTGCGCTAAGGAGAGTTGTCTATGTACTAGTGATTTCAGTCG |
| | | GAAATAGCAGAAACATGAACCTATCACATGACTGTCGAATGGAAAATTTGGAGTCT |
| | | GGAACATTCAGTATGAGATATACATTAATCCATGACTCAGAGGAATTGACCCACTA |
| | | ATGTTATTCTTAGTTGCAATTCCAGGTATGTCTAGAATTTGCAATCGGTTAGCCGT |
| | | TGTGTACTTCGTATCAATTTTCAAACAGAATACAAAACCACGCTAGTTAGCCGAAA |
| | | TTACTCCTAATTGTCGTCACTATGTAAGAGATTTAGAAAAAATAGTATTTGGTACT |
| | | ACTAAGATAATCGCTGTCCACTATAAACTTGTAGGTAGTTAGTCGAGTGTTCTGCA |
| | | AGGGTACATTCATGGAATTCGCGAGCAACGTTCGCTTCTCCCCAAATATTGATATA |
| | | AAGACGATCCATTCTATGTATTTTCGCACTAGTAAAATACCTATCTACTCGACTTA |
| | | CGCTATAGCTCAGGGATCTATTTGTAGGCATCCACAGCTCAGACGAAATAATAGAT |
| | | TTACGAACTGATAGCGGCCCTCCATGCCTGCTAATCATGTTCATACATCCAAACAA |
| | | ATCGTTTTGTTGGTAGACAACAACATAGCGATAATTTCAACTGGTTGAAATGGTTG |
| | | TATAGCTGAATATAAACGATCCCAAAAAATTCAAGATGGTGGCTGCACCGGAACGA |
| | | CGTTAATAGCGTGAGGAGGTGTTAAAAGCAACAAAATCACACCCGCCGTCTTCTAG |
| | | GGTAAGCGGGTGCCAGCCGGGTCTACTGGATAAGTAGATATTTAGCAAAGAACCTC |
| | | AGTTATCCATTTTCTGGTTACGTGCACAATTAGTTTTGCATCTGCCGGCTTTTGTC |
| | | TCTGGCACTTGACAAACCTAGCAAAACTCAACTGAGGGGTTAACACGCTCTAAGAT |
| | | TCCTCTTACTAGATGAGGTATTCATCTGCGTATCTGATTCTACGTTATAGGCTTTT |
| | | TCTCTCGAATACTAATGTCTGGACTGATCAATAAGAATTGGCTAATTGCGGAAGTC |
| | | AAAATAGAACCAATTATATTCATACTTCTATTATTAGTTCTAGGATGATTTTCCCG |
| | | ACCATCGGTAGTAGGAGGAGGTGATGTAACTCAGTAGTATTATGCTGAGTGATTGC |
| | | ACCTCTGATTCTATTAATATGGGGGGATGCTGCTTGCCTCGTGGGTTAGTGTCCGG |
| | | ATGAAAACCCCCCTAACCTATTCACGTATAGTATCCCAGTCAATTGAGTCAGTGAC |
| | | CTTAATCCTAACAAAAAATACAGAATGCTGTGAATGACCTCGTTCTTCTTATTGTG |
| | | CACGATCTGATTCGAAAATGAACGGTATAGAGTCTGAGCATCACGATATAAGAGAT |
| | | TCATTCTGTATTATTTACGAAAGGCGTAGCACCATTCGATCAGCGAGCAGAACCAC |
| | | GGGGCAGTATTGAATTTCCGTTTTTCCGATTTCAAAACGGCTAGAAATGGCTGCTG |
| | | GATGATAGATGCCCAACTCACACGGTTGAACTTGCTTATCAATTGTGCGGTTCATA |
| | | TCAGACATAGCAGTCTGCTTGGAAGATATTGAGTAACTTCAGCATTCAAACGCGCA |
| | | AAGCTATTGAGTTGCCCCTGATGCTGTCTATCGTGTATTAAGTGATCGTGGGAATT |
| | | AGACATACAACTTTACCTCTTCTAGCTTGTTTATAGAGCCTCACCGAGGTATAAAT |
| | | CATTAATTACCCAGGAGACCGGTTTTGCTATTACCTTGTAATGTTCAAAAAAGAAG |
| | | TGGAACACAGTGAAAGCCTCATTTCTCAAGCAAGTGAGTA |
| 39 | 40.40% | TGTAGACATTTGTCTTCAATCTAACCTCTTTCTCACGAAATAAGGGCTTGTATTGT |
| | | TCCTTCGTTTGTTTACCGCACAGAAACAGCTTCACTTAACATACATTGTAAGTGTG |
| | | TATTTCTCGGGGTACGTAACATAACGAAACTTAAAGCAATCAGACATACAGTGCCA |
| | | TTCCCTACGGTACTGTCTCAGTATGTTAATACTACTCATTTGCAAAAGGATGTACG |
| | | CACTTCATACTACAGCTGCTGACGGTGTATATCAAACAATTATATTAACGCTCGTA |
| | | GGATAGTTCACGTCCGCCATATCTTTGATTTAGGCTTCAAAATTCAGAATAATACG |
| | | AAATAGTCTGTCTACTAGGCCAAAGTCACTTAAGGGCTAAGAGTGTAATGAGTAAT |
| | | CAAAATAATAATCGTTGAGTCGTCAATTGGAGCATCAGTTATGGCATTAAAACATC |
| | | TAGTGGGTCGAAAGGATCAGGAAATTATGTATGGGTGAGAGTCGCTGCTACGGTAT |
| | | CGCTTTTGGATTGAGGGCTACTACACTCAGTACCCACAGTGTGTGTATTAATAAGA |
| | | ATCGCAATATGCGTCCTTTTAAGTTTTAAGGTACCCTACCTTTCATATCTAGTGGA |
| | | AATCATTTACGCCTATGCGACAAATTAGAGACTTTTATTTGTAAAACATTGGATGT |
| | | TGGAATGACCCTAGATGCATGTTAAATAGCACGTTCATTAGTGGTACACGCCTATC |
| | | ACTAACGCTATGGAAAAATAGAAGAAGCCAGAACAAGTAAACCTATGGTGACAAAT |
| | | AATTACATAAGGAAATCCCTCATAATTAGAATACCATAAAACGTTAGTTGTACTAT |
| | | CCGTAATCTACCTTCTAGCGTGGAATAGTTGAGTGTATTCTAGTCACGCCCCGTTC |
| | | CATAACGATACATGTAAAATTTACAGCGACGTTTAGGAACCCTACAAGGGGAGCAG |
| | | CAGCGAGGATAGCTGACTAGCCTTACAATAAGCACCCATACTTATGATTGACATGA |
| | | TGGTCATGCGGCGTTACCACTCCGCTAGCGTTACTTCTTTCGTCTTGTACCGGTTT |
| | | GGCAATGCGATGCAGCCCAGGTACCGTAGAGAAAGTAGCGATGTGTGAGGTCGAGT |
| | | ACTTTGTCAGAAAGCAAGTCGGATTGCGGTCCCATTTACCGCGACGTGCATTTGTA |
| | | CAGTATGACCGTTTTTTACCACTTACTGATGAGGCCAGACTAATAAACGATATTTG |
| | | GTCACAGGACAATATTACGGCCAATTATGAAATAACTGACTGGCCTATTGAATGAC |
| | | TAGGAATGTCAAGTCCAGACTCTAGCTATTTGGGAGGTTTATATGTTTGGACCGAC |
| | | TTGTGGGAGTTTGACACTACGAGTAACAAGATTATCCCTTTTTATGCTGCGCTAGT |
| | | TGACATGGATTGACGAGGTTATTAATATCCATGACTAACTCATCACAGCTTCCCGA |
| | | GCCGAGACGGATTATTTTAATCTCGTTGATCGATATATTAGGTGACGTGAGAAGAA |
| | | GATGTGTCGTAATCAGTAATAGTTAGGATCAAGAGGTTAAAAGAAGCGCCTTCTTC |
| | | ACAGATTCTCAGTATCTACCAGCACAGAGTTCTCAGTTTCTAACGTGTTCCGTATG |
| | | GATTTGCGCCACTTTCTGAATAAGTCTTATGAGATATACTTACCTGGTCCAGATGT |
| | | AGCAGCGAGTTAAGATTATAACTGCGGTTTAGCACGCAGCGTTTAAATACAAATAC |
| | | TCTTGACTGTTATAACGTTCAGGATTAGGAACAGGTTCCTCACGGATATAGAACCC |
| | | AATTCACGTGCATGAGGTATTCTATCTTAGGGGGAGGAACTGCGCTGGAGCTTGAA |
| | | ACTGACCCTCTAGGCGCTTGCTTTCACTGAGATCTATTCAAACTGACGTTTAGTAA |
| | | GAAATCATAAGACTTATCTACGCCGCCTTATAATTTATGTTATTAAAACATGATCA |
| | | TGCGATCAATTAGGTAAATTTCTTTGTGCCTTGCAATATG |
| 40 | 38.60% | CGAATATTTATTTTTCCTACGCACCTACACTATCGTGAAGTTCATGGTATCAATTA |
| | | TATGTCACTAGAGCCACAAATACGTACTTAAATCATTTACCTCGACTGAAGGTTGT |
| | | AGGCTTGGACATACTCTTGCCACCATTGTAACAAAGGTAGATCGGTTGGACCCGAA |
| | | ATTTGGTACTTTTAATCTAGAATCAGCAATATCCTACGGAAAGGCCCAAGAGATGT |
| | | CTCAATGGATGAGAGTGTAATTACCTAATTTCAGAAAAGAGAGTTTAACACAAATA |
| | | AGAACAGACGAATATCAATAAAGTGCACGTCGGGCCTAAATGAGCCCACAGCCTGG |
| | | ATAGATTAAGTGCGATACGTCGCTACCAACGAACAAAAGTATTTGGTATTATGACA |
| | | TCGGCTCCGACGGTATAGGATAGGAATAACTCCCAAACAATATAATCTTGGATACG |
| | | ATTAAGTTTGAGTTTGATTGATCCCATCAAACATTTGTTGGTATAAAGTTAATGTG |
| | | TGATCCAGTTAGAATTATATGAACATAGTGTTGTCACGATTTTGAGACGACCGTTA |
| | | AACATTATACTGCGGTGGCATAGCAAGTTCATCTCCTGACATTAGTCAGCATTTAA |
| | | TAGTAAGCAGGAGTACTATTAACACGCTCCTATAATCGGTTGCCTGTTGGGGATAA |
| | | TCAGAACATGAAAAACTCCATATTAGAAAATTACATAATATAGATCACGTGTATGA |
| | | AACCTAATACCGCGAATATAATTACATTATGATTGCAATACATAGGGTAGACTCCT |
| | | AGTTAACGTAAACCAAATAACCGACTCGAGAAACACAGGACTAACAATTATAATTT |
| | | ATAAACTAAGAGTGCTATACTAGTTACTGCCTGATACCTATGTTTATTTGCAAGTC |
| | | AAAAGTTTCAAATAGCCCTTGGCAAGCTACATGATGGGTGATTGGAGGTGGGACTA |
| | | GGAGTTCCGTCCTTAGTCTGAATAAAGAACATGATGTGCACCGATTTGTCGTCTAC |
| | | TCGGACGTTGTGGCAAGAATAAAAGTGAGGTATAGTACCGCTAGCCGCAGAGATAC |
| | | TGCCTTCATATGCGCCGATACTCTATTGTTCATAAACAGCAATGAGGCAGAGCACA |
| | | TAATCTTAATTATTAATTTAGTTAACGGCTTCCCAATTTAGCAATGAATAAATTTT |
| | | TTGAGGTGCATCTGTGATTAATTCACCCAGAAACGCTTTCGCGAATTACCTGTCAC |
| | | TATAGATCCTTAATGAATTATCTTCGTCGTCGGAACAATTATCGGACTTTATTTTG |
| | | CCTGTTTTATGTATCGAGTTAAATAACGGGAATCATAATTTTATATTACATCTGTT |
| | | TTGTATAGCGGATCTCAGTAGGTTACATCACTGTCGTCGGATTCAACAGCAACAAC |
| | | ACCGTTAATGAATATAGCTACACTGCATGAGTCCCAACAGCACTGGTCCACTAGAA |
| | | ATATATAATTATACGAATACTTTGCTATGTTCATGACCTGTCAAAGGAGAAATCTA |
| | | GTAAAGACCCACGGATATCGAAGAACATTGTAGTTCTGACTCGGTTTGAATGTCCG |
| | | GTAACTGCAGGTTCCCGTTATACTGAGCGGTCCGAAAATGGCAGTCTAAGTCCCCC |
| | | TACATGACGATTGCTATTTATTAGGTCTCAGAATATAACATTAGACACAAGAGCAC |
| | | AATAGTCGGAGTATGCGTTATCGAGACCGTATATGAGTCAATCGAACGTAGATCGA |
| | | TCATAGCTAACTAGGTGGTGTATCACTGACGACTTGACGATGTTTTATCGCTGATT |
| | | AGTTTATGATCTTGTAAAGATTGGATGCTACATATTATGGTAATTTTGCTACTTCC |
| | | CCCAACTATACCAAATGACTCACTGTTTATCAAAGGTGACTGGATAGGCGCTAGGT |
| | | ATATCCCGGTGCGCAATTATTGCCCTGGCGAGCCGAACATCTCGAATATGTAAAGA |
| | | CGAATACTCCCTAATTACCTTTTCGAGGTAACAATGAATA |
| 41 | 42.20% | ATCGAGTTGGTTTCTACGAGTAGCTGGCAAGCGCACATAGAACACACATTGCATGT |
| | | GAGTGGAGCGATTGCGAGACGAAACAACCTTCCAAAAGCCCAACGATTACAGTGCT |
| | | AGTTATCTATGGGAACTTATTCCCTTAGGGCCAAAGTCCCTAGGTTATTCTATACG |
| | | ACTCACACCGAAGAGGCTGTAAATTAACCCGAATATAGATGATTAGTCCTTTGTTT |
| | | GTCTTAGGGATGGCACCATAATAAAATTGTCAAATTAGGGTACAGGACTAGTTCGA |
| | | TTTCTTCTATCCGTCGTCCTAGGTTTATATGTGGCCGTCACCACTGTATCACATGC |
| | | CAGCTAGCAACAGTATGATGTATAGCGGCAAATCATTCGTCGGGGGCATGCAGAAC |
| | | GTCAGTTAACTTTAAAGATGAGACTACGTTTTGGTCACAATACAATGACTTAGACT |
| | | CATCTCTTAACTCAGACAATCACTTTTATACTTAGTGCAATGTGTCACAGCCACTT |
| | | AATGGCCTAGCTAATCCTTATAGTCGGTAGCTAGCGAGTTATAGAATCTTGTTGTG |
| | | GATAATCCTGCTCAACCTTGCCTGGAAGTCTAAGACCAGTACTAGAAGTTAGGCGT |
| | | CGGAGTCTGTGATGCTAAAGTTGTTCGGCCAACTAATTAGGGGTGTACCTCCTTGT |
| | | CTAATCCTCTTAGAAATTATTCGAGAAGGGTACAGTACCCCTCACAAAGAGAATCT |
| | | AAGTTACCGTCTGAAGTCTGAGTGATCCGTTTTGAGGTAAACAGCTGTTATACATA |
| | | CTTACAGCTTAGTCTACATGACCTACTAAGCGCTTCGTGCTCCTTACCGTCCCAGA |
| | | ATACCCATGGCTCGCGTCTCCTGCCGTACAATACGTAGATTTAATACTCGTAATGT |
| | | TTACAAAAAATGGCTCAGCGAATATGAATACGATATACAGTACCATATTTATGGAT |
| | | ACAAAATTTGTGGCATCCGCCTAATAGGGCTTTCCTCAGGGCTTACTCCACATACT |
| | | GTTCAACCTTCTAGGTTCAGTAAAAGTGGAGACCACGATGCAGTGTCCTTCTTAAT |
| | | CTGGCCTTATTTGTCGATCCCTTATCTCGCTAAGATTAGTCACACGACAAAGAGGT |
| | | CGTTAATGACGTATCTAGCCACAATCGACAGTCTTCTGGCGAAGATATCTACAAGA |
| | | GTCGTTGATTCGTCACTTTTAGCCTTGTAAAATTGCCCTTTGAATAGGTGACACCC |
| | | GAATGGATTGGTACTTTCGTAATTAACCGAGACTTTGGAGAATTGTCTCCGGCGTT |
| | | TCATGTGGCGAAGAATAGAGGTGACTTTGATGGCACCAGAATCTCACTGACAATTG |
| | | CTATAGACCTAATATCGGATATTTCTGCAACTTCCTAATCGAAAAAATTTCTACAA |
| | | ACCAGTCGCAGCCTTGAGTATTCGCCCTTGACATAGATTCACAAGATTGAGTCGCA |
| | | AATGGTCCTATGATAATGGATGTGTTATTGCTGGAACTTTATCATGATGCAAAGAG |
| | | GTTATAATATTTTGTGTTAGTAGCACACTTAATGCACGCAGAATCCTTAATCAATC |
| | | ATTAGCTGCTAATGAGAATCAACCGACCGTGTTGGTGTTACTGGAATTATATTCAG |
| | | TATCGCTCTGATCTTAAGGCCCTCAGCACCTGAGGTCTAACGAAAATTTTTTTAAG |
| | | CCCATTCTCGCAAGGCCACAACCATCAGTCTCTCGAGAACGACATTGGACCTCATA |
| | | TCCAAGCCTCCGGTTATTCACCGATGTATTTCTTCGAGTATCTAAAATCTGCCAAT |
| | | ACGATTCAAGAGAAGTTAGTATGCGGGATCATGTAGCGTACCTTTATATGAATAAA |
| | | ACATACCTGGTAGATGGAAACTTGGTGACCCGGGAGTACGTCATTCTGGTACTGAT |
| | | ACTTGAGGGTGAACATGGTGCGTGATTCCAGTATAGCGGTGAACCTACGACAATAT |
| | | GTGCATGGCATTGCTTATTTGGTGTATCGTTTTTTGAGAA |
| 42 | 37.60% | TAACTATATGGTGTCTGTTTACTACGATTGCATTAAGATTTCTAGCAATCTTCTCC |
| | | AGTAACTGCACTTCCCCATATTGTAGAAGCGACTTATGGAGCTAATCTTTCACTTG |
| | | GTTTAATGCTAACTGGGATTTGAGCACGTAAAACTTAACTCGGACCACTTTGTTGA |
| | | CATAATTCCGCTGCTTATATACCCATATTCATGTCTACGATTATAAAGTTCTTCGT |
| | | ATTTGGCTAAGCGTCTCTACCTAGGCTCAAGCCTTTTTAGCCAATCTGAACGCTAA |
| | | ACGGGTGCTAGCCTAGTGATTATTTAATGACGATTTGAGTTCATGGACGAAATTAC |
| | | ATTATTACTGTCTAACCGGACAACGGGCACGTCACAATAAGAAGGGTACAGTTGGG |
| | | ATCGCAGTTTATTCATGCTGTATGCCAATTCTACTACCTCTCGTCATCTTAATTCA |
| | | TATATAGCTGAAGGGCTAGCAAGTAGTGGATGACTATAATCGGGATTTAGAAGAGT |
| | | TTTTTCCTCGAACATTAGCCTTATGTGTCTATTTTGTTAAAATTGACATGCTAAAC |
| | | GATAGCTATTAGCTGGAGGAATAACATAATGTTGTAAAAGGTAACCAGCTCATCAC |
| | | TTCAGGAATCTTACTTCCTACGATGGCTGTCTTTTAGTCGACGTAAAGAAACCCAA |
| | | CCAAGGAATACTTAGACAGACAGGAGATCATCCTACAAAGATAGTCGATCTTTTAT |
| | | TTAGTCCAACGCTTACCAATGAATAGGGCTGTCTGAGACTCAAAATATTGGACCAT |
| | | GGGTTTCGCAAAGCGCAAACGGAGAACTATGATTTCTTGTTGTGGCAGCGTATGGT |
| | | CCCCACGGGTGACTGTACAATCACGGAGACTTTTATCATATAACGATAGTACATTT |
| | | ATCTGGATACCGGATCCTTCATTTCTCGGAACTCTATACTTACTTTAATTTAATGG |
| | | CCCGAAATCTATTATCCTTAAATTACACCGCCGTGGACTCGGAATGAAGATGAGTC |
| | | CGCAAGGCATACTGTTAGATCGGCTGAGATATTGCCTAGTGCAATCGATCTTTTGA |
| | | TGGTATTTGTGTACATTCTAATTCGAGGCGAAACTGTCAATAAACTAATGGGAAAA |
| | | GCAAGCATATCACGAGAAATATTCTAGGGGATAACATTACGTTTTCGGAACACAAC |
| | | AGGTTCGACATAAATCTTTTATCATATTATTTGCTTACAATTATTTAGGGCTTCCG |
| | | CCCATACTCAGTAGTTCAAATGATGCAAAGGATGTGGTGTCTAGTAGATCTCTTAA |
| | | ATTTCTATCGAATGGCGTAGTTACATTGCAGTTATTTTTACATGGCAAAATGATCA |
| | | AATTTGTACGCAATAGCAGTAACATATTCTCTGTAGTCTATATCTTTATGATTGGA |
| | | GACTGTTAAAAGCTGATATGACTAATCAAGAAAATATCGAAATTTGATCTACGACT |
| | | TAACATTTTAACTAAGCAGACATCATAACGTTTATTCTTCAACGGGCCGTTACTGC |
| | | TAAACATTAATCTAACGTAAATCGGAACTCTGCAGAGTGCCCGTCTCTTATTTTGT |
| | | CTGAATTTTAGAATTTACAAGGAGATGCTCAAGCCGAGTTAGAAGAAGAGAAATAT |
| | | AATGAATCCACCGAGTGTATGTTTATACATAAAGAACTATCTTTAGGCGACGTGCT |
| | | AGATCCCACTATGTTCATGTGTAACGCATTTATTGGTGGAACTCTCGCAAAATCTT |
| | | ACATTATTTCGCCATTACGTCTATACAAAAGCTAGATCCGTGAAGGGTCATAACCT |
| | | CCTTTAAAGGCATGAAAGAGGTTATCTAACTTATGATTCTATAACATCGTCACTGG |
| | | TGGAGTAAAAACATCTGTGATAAATACTTGTGATACTCTCTAACATCCCTGTAATA |
| | | TGATGATCATAACGCTTGCACCTATTAACTTAAAAGAAAGTTGTCTTATGGTGATT |
| | | CTTAAATAAAAGTGCCTGAGCCACCTTGTGTAATTTTTAA |
| 43 | 40.20% | CACAATAGTATAGGGACGTCTATTATTGAAAATTATACCATGTGGACATATTCTGG |
| | | ATTTGAATTTATTTTTTACGAACTTACTCGTCTCTTTGTCGAACTGATCGAACCAT |
| | | GATAGGCGGTCCATACGTGTAGTGTGTGCTAGAAGCATCTGTACTTGTATTGAAAG |
| | | GAACAAAGTCAACCATGCTGTTCACCAATTTGATACGAAGGAATGTCCTATCTAAC |
| | | CGGGCTTATTTTACAGGCTAAGTAGGTGAATAATGACAGGAAAAATTCGAATAAAT |
| | | CAGAAGAGTTTTAAGTAAGGCTCACTGGTCGAACGGTGATAATACTGGCGGCAAGT |
| | | TCTATGTAGCTTATTAGATAACTCTTCGGGTGAGAGAAAGAGCTTATAAATGTGGC |
| | | GCTGAAATCCGATGCCAGCTGTAGCCGAGTCGCGTCATCTCCTAACGGATCAGTTA |
| | | ACATTATGCTTACTGGACGTAAAGTGGCTTGTCTAGCTCTCATGCGCCTTGTAAAG |
| | | CTTTTTCTCACTGTGTTCGATTATAGTGCTCTCAGCCTACCGTTGCAAACAATGAC |
| | | TAGCGACTGAGATGACAACACGCCACACATATCGAGTGGTACCGTATTGGGAGGGT |
| | | AGTGGAGAGACCACCCGATATGGATAACACGTACAAGATGTGGTTAAAGAGCCAAT |
| | | CACAAATTGAGCGGCGATCGTGTCGACAATTTTTCATTGTGTAAGCATGCATGTAT |
| | | ACTAGAAATAGAGTAATACTTAGCATATACGATTAACTCTTGGTGAGATGAGATTC |
| | | TAGCTTTAAAAGAGGGGATACCGATAGAGTAATACATGTTCTTTTGAGCAAATGGG |
| | | TTGTTCGCCCTGATCCATGATAACGACTATTTCATAGCTCTAATTTAGATGCTTGA |
| | | CCCAGTGTAAAGATCCGTTTTAACTAACTTAGATGATAATGAGAAATAAAGTAATT |
| | | GACTACTTAGTACACTTTAAATCCTCCAGTCGATGTGTATTGTCGCTATATCGCAA |
| | | CCCGATGTTCACATACAGGGTCCTGACTTTGGGTATACCTTAGTACGTAACAATCT |
| | | CACTCACAATCAATCCAAGCGCGGTTACTATGTTACGACGGGGAAGCAATACACAG |
| | | CTAGGCGTGCAGTACTGCTCTTAGCTCTCCGAAATCTGATCTAGATGCCCAAATAA |
| | | TTTTGTTTCCAAAGCTAGCGAGGTTTTACGACCAGTCATGACAGATTCTGCAGTTG |
| | | AAGCATGTCACAGGTAAGCAAAAGCGTGGAACGGATGGAGCGAGTAATCAATAGAA |
| | | CTTACTTTACGAGCGGTGTTACAAAATTGGGTATAATGCACTAGCCGACATCGATG |
| | | GTGTAGTGAATTGGACTGGCACCCTCAAGGCCTCGCCCAACTCAGTCTCGCTAGTT |
| | | TGCTACCTGCATCCTATGAAGCTGTTTTTAAAAATATCGATTTCTAGCGGTAGTTA |
| | | AACTATTAGGAAGGGCTAAAACAAAGTTAATTATACTTATGTGAACTTACAATTTA |
| | | TATATTAGAAAGTGAGTAAGCATATCTGAACAAGCATCATCGTAATGAGGTCGGTT |
| | | CGAAGTATAAACTTAAGTTAACGACATCTTCCAATACCATCGAAGTCTACTAAGTA |
| | | AGTTAGGTGCTTAATGATCATTCATAGTGTAGCAAGTCCCCGCAACTAGATAAAGT |
| | | CAACGACTTAGGAGTTTAGATAGAATTGTGTACCACTAGCTCGCTACAATTGGTTT |
| | | GTCTAGACTTAATCCCTTACCTGTTGAGACCGACTCTATTTCGGTAAAAATCGGCA |
| | | AAATACGGTAACATTGTCTGCAGTCTGAACACAGACTAGCTTATATACATGGATCA |
| | | ACCATCAGGTGTGACTATGTTTTATTATATGAACTGTTACCATGGCGCCTACGACA |
| | | ATAGTATATTTCCATTTCGGTTACCAGTTTTTGTCTACTTTATCCATTAAGTGATA |
| | | TATATACATGTGTCCAACGTTATATGGACAGCGTTGTGCA |
| 44 | 41.90% | TAAAAGAACGGACATGGCGCACAAAATGACTATGAGGCGGTTACTTCTGATGATCA |
| | | CACCCTAGTTCTTACTCAGGCTATTGTACACCCTGCCCTCTCAATATACCCGGAAA |
| | | TATGCATTTATACGGCAATCGATCTTGAATCCCAGTTCGAGTCTTTACAAATTCCA |
| | | TCGTTTACTACGCAACGTCATGCTAAATAACACCTTCCCATATATGTAGCGTGGGC |
| | | GGGACTATTAGAGTCACTTTGTGCTAAACAGCCGGTAAGTATAATAGTTTACTCCG |
| | | GAAGGTGTCAATATGTTTAGCGACTGTATTTTGGTACTTTATCCCTAAACTTAGCT |
| | | AATTTACACATATAGCAGCTGGAGGAGCAAGGTATCATTTAATCTTGCTTAAGACC |
| | | CTAGTTTGTACCCCTGTCGCACACTAAACCCAAAATTGCGACATTGAGCCACTTAG |
| | | GCCACATTCGTTAATCTGGTAGTTACAGCACAATGGCTATAATATACAGATACGTC |
| | | TAGAAAAAAGTTATTTAATGCATAGCTTGCATAATCGATTCTTTAAAACAGGGTGG |
| | | GGAGCTACGTATCTAGGATTTTATTCTACGTCATGATAACGAATCTTCCTGAACGT |
| | | ACTAGATGGCGACTATCGGAGAATGATTTAGAACGCCGGGTGTGTCTTGATGATAT |
| | | AACAATAAGTACCACGAAAAGAATGTAAATAACTTGATATCGACTGTCACAATTTG |
| | | TTTGTATCATTGTTCGTATCATTATGCTCCTGCTCGTGTCGCAATTCCCCTTTCAC |
| | | CTTTTGGTTCTTTATACACAATCATATTATAGACTTATACGGAATATTGGTTGTAA |
| | | CTTAGAGTAATACCGATTGAACCCACATGTCGCTGACTGCGACGCTACGGCATCTT |
| | | AAGCCGATATATCGTCGTGACGTAACTAGGAGTCCGTAAGCGAAGAGTAGCATAGC |
| | | GATGATCGTTTCAGACTCGGAGTATTAGAGTTACCATGCTAGCCACATAGAACGGC |
| | | CTTCCGTAACCGGTGGCACTCGTTCGCAGTGGGAAGCCCAAGTTAGAATAAATTGC |
| | | TAAATCTGATTCTCCCGTCTGGACTTCGATCTTCGAGCTAGAGTGCCACTACGGGC |
| | | ACTAACACATTCAACGAGTTTCGTCGGGTGGCTCGACTATCGGCACGAGTGTTGCT |
| | | CTACGAGAATACCTGCCTTCCTTACTGCGATTTCTCTTTACGCTCTTCCACTGGTG |
| | | CCAAGTGGCTGTATATTACTGGTCGAGTAGGGCTCGCTGATTGTCGTGATTCAAAA |
| | | ACGCAACTCTAAAATCCATACCTTTGTTGAATACCTTTATTCTCGTTATCATAGAG |
| | | GTGTTCGGGCCCTCACTATCGATGGCAGATATAGCTTCTCCGCTCGTACTTTCATA |
| | | TAGATGTTCCCCAACAGCTTTAAAGTTAGAATGATCCACTTTCAGGGCATCCAGTA |
| | | ACTCGAGCAATTATGTATGTAACCGATCTTTCGATGATAGGGGATAGTACACCTTA |
| | | ACCCTTGTCCCCGGTGAATTGCGGCGACACCATGCGGTAGGCGTATGTACGGTGTG |
| | | CCCTTAATTAACATCGCTACTGTACTACACGGTTAGGTCGTTTGAAAAGGCAGCCA |
| | | TGAATGTTAAGATCTTATTTTAAAATTGATCATTTACATTTAGCTGCTTTGGGGGT |
| | | AAATCTACTGATCCAGGTATTAATCTCTTTTGTATAATGTACCAATTGTAGTAGGT |
| | | TCTCTATGTTCTTAAGTTTCATTGTCGATAATAAACTAATCGGCAAAGGAAGAAAA |
| | | CTCAATAACTTGTATTGTACCAAAAAAGCGGGGGCTATAGTTAGATCGGTGACTCA |
| | | CTTTCTTCGATATAAGGGAAACCCACCGTATAACGACGGTGATCTTAAGCCTTCTC |
| | | CCAGGTTAACGTATAGCCTACAAATGAATGCATTCAAAATGTCGTAAGCCTTTTAC |
| | | CTGGAAAGCACAAACGATAGCGCATTTCCTTAAAGTACCT |
| 45 | 38.90% | ACTTGCACAGAAATGACAAAGACGTCGATTCACGATAAGGCATTCCAATAAGTATA |
| | | ACATAATCGTGTTTCGGGGCGCACAAAATAGATACCCAAAAGAGTGTCCTTTCCAC |
| | | TCGACAGTAGAGCTCATAGTTCCGTGAGATTCTTGCCTCGTAACTAGTAGACTGTC |
| | | TATCGCAAGAATATCACACCCAATATTTAACAACGCTCTGACGTAGTAGTGGCTAC |
| | | TTGTGCGAATAATCTAGTTTCTCATATTTGCGATTCAACTTACGGCTAAACGGCCT |
| | | CATAGTTTTTCCCTATTTTGAACATAAGTCGCTGTTAAGCAGAGTGATACTTCCCT |
| | | TATTTAAGTGTAAGATGTTAAACACTAAGCTAGAACACAGTAAGCCCCCGTATCTT |
| | | AGACGTAATAGCCCTGTTAGATTAAAGGATTGCGATCGACATACCAACAGATGACA |
| | | TTAAAGCAAGTATAGCTTCAATTCCCGCCACGGTAAACACCTATCACGATACAAAG |
| | | GATAGACTTACCGAGTACCGTAGTTAGTAACCTCTAAGCTAGTAAATCAAAGTTTT |
| | | CGCTAGTTATTCATAAGAACAAAATTACAAAATGCGTATTTACAACTCATTTACAG |
| | | TGATGAGACCGATTCTAATCCAATCGGTGTTAGTTTTGCTTATCTGAAAATACTGT |
| | | TAGAAATGACGTGGCTGTTAATCAATGTATAACGTGCATGCGCTGAATATCAATCA |
| | | TCAGTATCGAGGAGTTGGCATACGCGGGGGCTGTTGTTAAAAATTGATCCGAATCA |
| | | TCTGGTTTACTCCACTAATGGATTAAGCCTCCTCAAGGCAGCTGATGTGAAACCCA |
| | | AAGATGTCAATTTGATTTCGGTAATTAATTGAAATCCCTGTCCTGAGCAGACTATA |
| | | AACAGATAACCGTATGGAAATCTGATTCCTTAGACGTTTTCAAATCTATTCAAGTA |
| | | AATTTTTACGGGAATCTTAAACGATATCGTTCCGTGAAGTAATTCAAAAAACGGTC |
| | | TTGATCTTATAATTCACGTTTGATACTAATTTAGTCCTCCGCTCCCTAATGATTTT |
| | | TTACGAAATGGTCCAGTTTATTGTTTTTAAAACTCTTTGGAAAATTCGTGTATGAG |
| | | GATGATAAATTGTTCGATCAACGTTTGTATACTTAGATCTCAAGCAAGAACTGTCA |
| | | GCGACCTGTCGTTAGGTAGTTTGTTGCCTGCCACCTCGCGACCTTAGGAAAGGAAG |
| | | GTAATCTATTCCTTAATACGTACTATGTACAAGAGATGCAAGAAAAGGGCAACATG |
| | | AGAACGGTTAGTCTCTTTGACCCTCTTACTGGTTAGTGAATATTTTTACCAGCTGC |
| | | TACGATGCAGGATATCTGGCCCTTTGACTGTTCCATGGACACGAGCCCGAAGGATA |
| | | TTTATTTAATCGAGAGCTGTATTTAGTATCTTCATAGGACTTGAAATCGGATACCG |
| | | CTGTAATTGTGGAACCTCATGAGACCTCCTAACAAAACAAGTATCGACCTGCCCTA |
| | | TCTCCGACATTTACTCAACTCTACCCCCAGGTTGACAATTTAGGATGGTGTCTATG |
| | | GGAAATATGATTCGTAACGTGCTGCCTCAAGAATAGGTTATGAAAATATATATATA |
| | | AAATTCTATGATAGTTCCTTCGTCTCACTCAATACTAAGTCGTTAAGCCAACTAGC |
| | | TCGGGCGGGCTATTAGTTGCCATATGAGGATCCATGAATCAAACAAATAATGCAAT |
| | | TCTGCTAAAAAGTGTGTATATAGAGCGTACACACAAGAAACAAAACTGACCGATCC |
| | | GACTTAACCATTTCAATATAATGCTGCACCCTTGTCCTCAATAGCTTGCAGGGGGC |
| | | AATTACGTTTGGAGTCTGGTTGTGGTAATACTCGACTGTCCTCGGCGATATAGAAT |
| | | AATTATAGAGTGTATTATAGCACAAATTATTAATAGATTCCATAGCCTGGCGTTAC |
| | | ATGAATATTCTCAGTTAAAGCATTTGAACGATCAAGTGGT |
| 46 | 40.60% | AGGAACAATGTTAATATCAAGTCGGGTCCAAAAAGATGTGTAAAGTTTGCGAACCG |
| | | TTGCGATCTGTTTCTGTATCGTCTTACACTGTCAGGGCACTAGGACTCACTACGAC |
| | | TCATATGTACATTGTTTAGCTCACTCCGAGACGCTTAGTGAATCGTTAATAGGTTG |
| | | ATTTGTTATTGAAGCTGTCTGACTTATTATCTTCTTAAACGACTTTTTACGTATTG |
| | | GGAGTCATAGGCGTTTTACAGATATCCGCGTCAGTCCACGACGTGGTGCTCTATCG |
| | | GATAGGTACAATCAACAAGAATGATTATTGCTCATCTTAATTTACTATGTGCGCCG |
| | | TTTCACCCCAAATTCGCTCAAGCTCAGACCATTGAGGGCGGAATAGGATTGAGGGG |
| | | TAGTGAGGCGCTGCTGTATTAGGCAACCCCGGTGGTTCATTTGAAAAAACAATCGC |
| | | GGAAACAACTCTAGGCCTAAGGGGAACAATCGCTTTGACTATGAGCTTCTATACCT |
| | | TTGAATATACACTTTGCGTGGAGCTTGGCGCGACTCCTTTTGAGGTAATGCGATCC |
| | | TACCCATTTTGGGTTCCCTCTTAATTATATTATCGGCTTTTGTCACCATGATCTCA |
| | | TAATACTGATAAGTTACCCCTGATGTTACGACCCCGCAGCCGTTAGATATTTTATT |
| | | TAGGAGGACCTACCCAAGGCCTATGATCCTTTCTCTATATCACGAGGATTACAGAC |
| | | AAGAGATGTGTAATCCGCCCAAGTTACTCTACTCAAGGTTGCGCATATTAGGGGAG |
| | | GGCGTTTGACAGTTGCAGTATGCCATCTTGGAAGGCAACAATAAACGGTACACAAC |
| | | TTTACAAATATTCCATAATTGTTTCTACTTTTCATTCATTCATTATGTATCCCTCT |
| | | ATACTTATAAAACATGTACGACATGTCCTGTAGAGCGGGACCTGTTCCCGCTCATG |
| | | ACAGACGAGTTATTTGTCTCCGACGTATCATCCATCTTTAAATATTGAATAGCAGC |
| | | AGCATCAAGTGTGGATAAGTGCAAGCACTATTAAATCCGCGTGAACTTTCATATGA |
| | | CATGAGAATCGGACTGTCTGTTATCGTAAATAAACCCGAGATAATGTTAAAACTAT |
| | | TCTAATGACTTCATGAAGCAGGATCATCTAAAGTTATCACAAGAGGTGGTCTTGAG |
| | | TCTTGCAAACTTCAGAAAACATTTACAAACGATTCAAATTAGCCTAAACCACTTAC |
| | | TTAACCACTCATATTCCACAAGTTACGGTTCTTTAGAATATTAAGGTGTAATGACC |
| | | CATCGAGCCTTATAGCTCGAATCAAGATTAAAAGAATATTCTAAATGACCATACCG |
| | | GTTACATGTGTGGGCGGAGTCAAAAGTTTTTCTGACTATTAGGTGCACAAAGGTGT |
| | | TCAGAACTTAACCAAACTCTTAGCACATTTGATTAGCTAGTCAGATTAAGGTCTCC |
| | | ACTTTCTTTTCTGTGGTAGTTCGGTAAATTGATGGGCATTAACAAACTTAAGGTTG |
| | | ATTACAATGGGGGGTTATCGGATGGTTATTGTAATTGACCCGTCCATAGATTTGCT |
| | | TAAAAATCGCATTTTGAATACATATCCTAACTTCCAAGCATTACACAGCGCTGCAC |
| | | TATAGAGCTAGGATGACTGTACAACCTCGGATTATAGCTTCTACGTAAGGCGTGGC |
| | | CGTGGCTGGTATAATAGTGGGGTGGAGGGAGAATTGACAAAAAAAGTTTATCATTT |
| | | AAATATTAGTAATGGGGTTGTCGTTCTAGGACCGTATTTCGCGTACTAAGTCACAT |
| | | ACCCTTATATATTTTCCACAGCAAGTCTATCATTGCAAGCTGTTAACTTCATTCCG |
| | | GCGGCTGCTGAACCAGTATCAGTTGGTCCACAGAAGCTAAAGTTAGCAAAGTAATA |
| | | CACGCCAACCTACTTATATATGTATATCGTATAGCTTAATTGAGATGTCGTAGCCA |
| | | TTACATGCTGAGCCTTATTTTTGACCGAGACCAGGTACAC |
| 47 | 39.40% | TTGGACGTCGAAATTATTTTTGATATACGTGTAATGATAGACTAAAGGCAAAAAGA |
| | | AGGAGTATAAGTCTAAGTTCGAAGAGGCGGATTTGGTTATACGTCCTGCACCTCTT |
| | | GCCAGACATTCTTTTAATTCTTGTGACCTGGACTTGAAGTTCCTTTTTGCGACCAT |
| | | TTGTGGGTTTAGTACGAAACCCCCATAAGCAGTTAGCATTAAACCATCAGGTTTGA |
| | | CTCGCCACATTCGCTATCGCAAATGCTACTAATTCATCTTAATCTGACCCCCCCGG |
| | | GAAGGAAGCCATTTAATAGATAATCTGAGTCGTTCCAGAGATGTACTTCTCAGATA |
| | | AACCGTGAACACTATTACGACATATGCTGAATAACCAGTATGTATGGCTGTTGTCG |
| | | ACTCTCATTCCTATAGTGGAGAGAACTGATACATACATATTCCCTACACGGATGTT |
| | | AAAGAGTCGCAGGACCTGGTGAGGCACTGGATCAACAAGTTGCCAAACTGAGTGCC |
| | | AGTGGAGCTAATCACACCTTCGGCTCTGCGTTACATGCGTTAGTGAAGGTCCTTGA |
| | | GGTGTGCCAGCAAAGATTGTTAACATATAATCTAAGGGATTATATGGTGTATATGG |
| | | GACTGAAAACCTAGAGGTCTGTGGGGAAAGACCGTACAGTCCCTGACCATCACAAT |
| | | AAAAAATAGCCAAAATAGCGTGCCATTCTAAAATTTTAATTTTTAATCAATCGCGA |
| | | CTCCTTTGGTTTCATGCTAGTTGATTCTATTTAAGAATCCAAGTGAGTTTTAATCT |
| | | TAACCCTAATGATTTAAGGTTCCAGTAAGCAAATAAACGACTCGCCGTAAAGCGAA |
| | | ATTGATCGATACGTTTCTTGCTTTATTTTTGGGTACAGCAATCCTTCGAAATGTTG |
| | | GCTTCGTAATTCCCTCCAGTAACTTAAATCAGTTAATTTGCATTGTAAGAAAACAG |
| | | CAAGTGAATCATGTCGCCGCTTCAGTAACTTACTGCAAAATGAAAGCCTAATAAAT |
| | | AGTTACCCATCTATCTAAGTATAAACGACTTTTGCTTATGTCCACCCATGCTAGGC |
| | | TGTGAATCCTCTTACGTATAACGTGCTTTGCGTGTACTTTCGAACTTTCTAAGTAT |
| | | CAATCGCAAATCGAAGTAACTTACCACCGCTCGTAGGAATTGCATGTTAAAAAGGG |
| | | TTAACTCCCTTCGCTTTGTCGTTTCCCAACCTGATGAAGGAAGGTGAAATACAACA |
| | | TATGGAATGATATATATCACAAATACACACGACTCTGGACCAGTGCAAAGTAGTTA |
| | | TAAACTCAAAACGCCCCCGACATACATTAATTCTACTTCGAAAAATATGTTGCCCT |
| | | AACGAAATGGTTTGCCTAACAGCGGCAAAAGATATGTCGACTCGATTGTATTTAAA |
| | | TCGATTATTAAGATTGGGATGAGGGCCACGTAGCCGAAACTGCAACATACCGAAAT |
| | | GGGCGTTACAATGCATTAATTATAATTTATTGGCGCTCAGCCTTAATTAACAATCT |
| | | AGGCGTGCTCATACTGTGTACTTTAAAGCACCATTTACATGTCATAACAGATTATT |
| | | GATGTTACGTAAAATTCATAGTATACAGTATCACCTCGATCAAATTCATATGTTTT |
| | | TATTTTAAACAAGAGTACTCCTGTGTCGTTCTGAATTACTATTAGTCAGGTGCGTT |
| | | AAGCTCTGCAGAACGATACCGACTATCTGTGCATCTACCTGATTCGAAAATGAAGG |
| | | CGATTGGGACTCTCCACTAGTTCTGAGTTGTCCTCCTCGATTTACAAAAGATAACT |
| | | TCAGCTGGATGTTTATCGAACGCACAAATCTTAACAATGGTTTAAGTAGCCGAATC |
| | | AGATTCGCCATTCAAATCTTTGCTCTAGTTTCATCAGTCCGAGTTACTCTCAAAAT |
| | | AACAACCTAACTCGTCTTGCCTACACTGGTTCTGGGTTTTATATTTAGAGACATAA |
| | | TCACGAAACTTCATGCACTATAGAAGGCACCATGCTGTTC |
| 48 | 41.40% | TGAGCTTCGCTTTTTCCAGAGTCGCTGACTAAAGTGAAGTGTCTAGTCGTTGTCCA |
| | | TGCGATATCGGGGTCCATCAACTAGAATTCATTTACGGTACGCGTTGTCATGCCTT |
| | | ATATTTAGCAATAAGACTAACGGAAGCTCCTCTGGAGGGAAAGTAAGAACGTCCCC |
| | | CCGGGAACATACCTAAAATAAAGGTGCATGAACCATCACGGAGTGGAGACGCAAAA |
| | | GATCAATTAGTACAAATCAGCAGGAGACATGCAAAGACCGCGCCCCTTTCTTTTTA |
| | | TACCATCTTAATAGCCTTTACTGATCGTGTATGTTTTCATCGTGCACCTAATTATG |
| | | GAAATTCTATGAAGCTTTTGCTCCTAATCGTTTAGTAATGCTCTCGGATGCCACGT |
| | | TATCTTACTGAGAAGCCCGTGACCAAAGCATGGTGACAATAGAACCAATATATATG |
| | | AAAATACCGGGTTCGTCTGAAGACTGTGTAGTAACAAAGGTATTCTTGTGAATTCA |
| | | CGTTTTTAATCTCATCTACTATCGGATATGACAACAAACTCTGATTAGGGTAATAT |
| | | AAAATTTACCGTTCGGCCTAATTAAAGGACAACCGGTATGTAAAACAGCAACATCA |
| | | CCTAGCACGAAATTTACCTATGAGTGTGGAATTCGTTAGCGCTGTCGACGTGCATA |
| | | ACCTACGGGTTGTTGCATACGGGTCAGTGGGATAATGTTGACTCGGTCCTTAGTAA |
| | | AGACTAGCTCTTCTTATTCTTGCGCTTGTAACTGACAAGTCGAGTTCACGTGGGCG |
| | | CAGTAAAGTCGGGAAGACGGTAATCGCAAAAGTTCGGTAAAACTAACAGTTTTTAA |
| | | CGAGTCCGTAAGTTCAAGGGCCTAAATAGCTGGAGGATTTTAACGTCTAAACATTC |
| | | GGGACACAGTGTATGACCCGCATAAAAGGTTCAAAGAAATAATACTTAGAGCCGTC |
| | | GTTCGGATCTTATATGTTTGAATGAACCCTTAATCACCCTATAACATGAAGCTACG |
| | | ACACATTAATCAGATCAAAACCTACTTAGAGCTCGTCCGATACTACAACTTGAAAT |
| | | CTTCCACCAAAACTAAAGGGTCCATTATGTCAAAATACCATTTCTATTTATATTTT |
| | | AACCATCAATTCGCCTATACCCCTAATCAGCATTAATCTCGCTTAAAGATGGTAGA |
| | | GTTAAATACAACGCAGAGCTTTTATACTACCAGTGATGGATCACAGGATTGCGTTT |
| | | CAAAAGGTGATAGCAATTACCAATGACCTTTGACAGTAATGTTACATCCTAACCGG |
| | | ATTATTTGGAATACCCTCTATTTGCTTTCTGTTTAGCCGACGCCTGTAATTGTCTA |
| | | CCTGCGTGCGTTGTGATGCCGGTCCGCTCGATTTAAGCACTCCGATATCTCATGTA |
| | | GGTGTGGACTTTGGACAAGGGGAAATAACTCTCAATGACAATCGTACTGCTTATGT |
| | | TAGGCAATGCTGGCATATGCAACTCTGAGGCTAACTAAGTTAGTCTTGTCCGTGAT |
| | | CTCAGAACAGTAACTATTTAGTTGCTTGCGAGTATATTTCGGTAGAGACGTATCTT |
| | | CTACTAAACACGGTTAAATATTTTTTGGTTATCTCTCGCCCGGTCTAGTAGTGCCA |
| | | TAACGTTTACGAGGTCATATAACTGTCATACATTGCAAGGCGCTTTATCTCAATTG |
| | | TGAACAAGTAATTATAGCCATGATACAATTTTTGGACGGAACTTGTTTTATCTAAA |
| | | TCGAAAGAACCTACATTGCCTCGGCATAGACCTCGGAAGCAGCTAGTTCACTAGCT |
| | | GCTTCATGATGGTCCAAGCTTGTGAAAGATTCACATAAAATCAACCTCCGTGGGAG |
| | | TCTCCGATGGACGAAGCTGTGTGACTGGATATTATCTCATGATTGCGTCACCCTTA |
| | | ACATGTGTGAGGTAGAGCTAACTATAGAAATACCAGTCGAGTTAGCGACATAATGC |
| | | GAATTGATCCGCCTGTCAATTCCTCCTTATACGCGCCGTT |
| 49 | 40.00% | ATTGTCCATTCTTGTATTTGAATCACTCCCTAATGAACCAAACTCTCTAAGCCCAT |
| | | TCTTGTAGTATTTAACACACATGACAACGGTCCAATTTTCATGTATAGTCGGAGTA |
| | | ACGCGATATACTGAATCTTCTGACTTATCAGACATATAAGATGTAAAAACAGCGGA |
| | | TCAAAAGTGTTCTCTGCTGGGTGAAAAATGACAATTAAGCGTGGTATTATCTCTGT |
| | | AAATAACACAGGGATTTATATGTAAGGATCGCGCCCTCATACATTCATTAATTCTC |
| | | ACTCAGACTTCCCTCCTTCGGGCTACGTTAGATTGAAATGAAAATAACATGTTGTA |
| | | ATCATTAAATAGTACATACTGAGTTTTTAAAGTCGAATACTACAAAAAATATCATA |
| | | CTTTTTTTACCAGTTCAGTATTGGAGTCGACACATGATCTAACATAACAGAAGACA |
| | | TAGCGATGGGGATTATCGACCTTTTTATGGGTAGTAACAGGTGGTTGCCGGATGCA |
| | | CTAGCATGATCAGGTCTCCTACTCACACAGTCCTTCTGACTGTTAGGTTGTCTTTG |
| | | CTTATAAAAATACTCGGATTATTGCGCCACAATTATTTGATCAACGAGCTTCTTGG |
| | | AGAGAATAAAAATATTACACTTCGGATAGATAATACAGGTTAGGTTCTCCTATGAA |
| | | TTTGAAGATCCCATGTTCGTTACCGTCCAAGAGCCACGGCTTGCTTGCTCGAAATT |
| | | AAAGTGGGCATTCGCGCGGGATGGGAAGTACCCTCAGTCTTGACAATTCCCATCGT |
| | | CAATATTAGAACGGTGGATTCGCCATCACCAGGAAACGTATTGCTGATGATGATTT |
| | | CAATACTGAAGTCGTACACTTCTCACCCGGAAACGTTAAAAGGACGATAATGACTT |
| | | AATTGAGATCATCGAGGTACGAGCCCATGCCTTAGGTCGCTTCGTAGGGGTCCTCC |
| | | TTAAAGGAGACTGTTTCTTACATGATTTGTTACTTCGTTGAAAATAAATCATGGAT |
| | | CGACGTCACCAATTACTGGGGTACCTGAGTATATAGCGTAGAACGTGAAAGTGATT |
| | | ACACCTGTATAGGAAATGATGAGCTCGGGGAACCATAATGAATTATAGTGTAAAGA |
| | | TAAAAAACTTGCCCCGTGCCACGAGAAGGAATGTAGCAGACAATCATGGGGACATT |
| | | GTAACTTACCCAGACTTTAATTTCGTTTTCACTATACCACTCAATTATGATGTGAC |
| | | ATTCTGGAATTGATAGCGTATGTTGCAGCCTTCTAAACTCAACACTGAGCTCCTTA |
| | | AGGGTTATTATGGTTATATTTGAGACTATAATATAATCCGAGTTCGGTCGAAGTGA |
| | | GTAATCTTTGGAGGGTTTAGGGGGGCAGAATTCACTATAAGCAGCAGAGATTTTCT |
| | | TAGAAAGAGCCGGGTCCCGTTCCAATAAGCCCTACCGGACGTTTATAATCATTGGT |
| | | GCATCAGTGAGGCCTTCTGTTCATCTTCTATTCTGCTGTACCCTTCTTGCACCAAC |
| | | GCGTTGGATCCTTGTATCGAGTCACTGCCAGGTTTGTGGATTTTTTGCAGCCCACC |
| | | CTACGTTATATCTTAACAATCGGATAATTAAACCAAGCTATCGAATGCTATGAGCT |
| | | ACCACAGATTATCATCGATTGTTTTCCCTATCATTACGATCCCTGACGGACTACTT |
| | | AGTATGTCCTTTTCTTAATATTCGTTAAGAACTGGAGTACAGGCTGATTACACAAC |
| | | CAGTAGGATTAGGATTAAATAGAGAAATGTATCCGGAAAAGCGGAGTTACTGTTTG |
| | | GGTCTTTAACCGCGAATCGCGGTTTTTTTTCTAATATGCAGTGATCCTTTATTTGG |
| | | TTACTGTACATCTGCTGAACACGCTATGTGGATCTCCCACAGTTGCAAGTGCAAAA |
| | | TATTAATAAATTAATCACAATACAGTACAGCTAGATTTCATACTAAATGCTGATTT |
| | | TTGACCGCACCCTCGAGAGTAATTCAATGACGGCCATGTA |
| 50 | 38.90% | AATCAGAATGAGCAGATGTAAAACATATTTATGTAAGCAGGTTATCCCGTATGGCA |
| | | CTCGTTGCTCTAAGTAGATGTTTTTGTCTCGGGTAACTTATGTCCCCATCCTCAGA |
| | | GTGTATTTACTTTTATTTAACCCGACGGTGAGAACATACAACGGGTCAACAAGACA |
| | | ATACGACCATTATACTGCTAAACTCTCTTCCTCAGGTGCTATATGAGTTACGACAC |
| | | AATTTTTGATGTTAAAGTCGACCCTAGCTGCTAACTGAACTTCTGGGACTTAAAAC |
| | | TACCAGAAAGGATGAAGAATTAGTTTGGTCAATAACTATATACGAAACGCCCTGAA |
| | | GGAAGTCGTATTAAATTTGGAGTGCATAAGACATGGTGAGCGAAAACTAACACCTA |
| | | CCTCTTAGATACAGATTACTTTTAGTTATCTTCTGGTCTATCGTTGATCATTCTAA |
| | | GTTTATTCAGCACTAGAGACTTTTGGAATACGACTGCCAAAGCTAGTATAGGATTA |
| | | TCTAAAGATCATTATTATTAACGGATAATGCGAAATTTGCTAGATCGTATATACTA |
| | | TTAATGCAGCAACTTAACTAAAGATATATTTACAGTGGGGCTTATGCAACCGGTGA |
| | | GCCCTCGGTTCTTTATGATTCGTCAAGTAAAGTTGCACAACGTTCACGATTTAATC |
| | | TTATTCTTTGATCTTGGGCTGATGTATCCTCATTATTTATGATAGAAAATTGATTG |
| | | GTGCATTTGATTCGCCCGATACTAGACCCACAGCTGTTGTTCGATCCCGTATACAA |
| | | TGAGAGCATGTTCAGATCAACAGTAGGTGTAACATCTTATGTTCCGAGCCTTCTAG |
| | | TAACCAACGAACACCTGGCAAATGAATTTGCCATCTTTCCGCTGTACGAATAGGGG |
| | | TAATGTGCCCTTGATTTAAAATGTTATCGATAGGGGAACTACAGATACTGAGAACT |
| | | CCTGAAACGACGTTAACAAACCTCCTGCAAAACTTGCACTCTTTGAACGAGGTTGC |
| | | CTAGTTTCCAGAAGTAGGTTCTTGTCACTTGAATTTCGATGGAATTCTCCTTATCT |
| | | ATCCAGTGACGAGGAAGAAGAAATGGGTTTTTACAAGGACTAAGTGTTTAGACAGA |
| | | AAAACTAATCTTTCAGTAAAGGTGAGAAGTGATTTTGCAGAGGGAGATTGTGTTAC |
| | | GAGGATAGTACTGACGTTTATATGAGAAATAGTTATCGATAATGTGCGTGTCTTTA |
| | | CCAAGGGACTGACCAACTGATGTGGAAATTTAACTCTTCATGATCACATAATTTCA |
| | | ATACGTTAACAGTTAGAAGCGGTGATCTTTACAAAGTAGACAATGAGTTATTGTCC |
| | | CATAGCAATGCCTAATGTCGAGCGTGCTTCAAACAATTGAATGGCGTTATTTTTTG |
| | | ATCCTTAGGAAACAAAAACCAGCAACGTAACTTATTCTTGTATCTTCATGTAATCA |
| | | CATTACCGGTATAGAGATGGTTTTACATATACGCACGTTACTTTGAGATAGCGAAG |
| | | CATACGAATATACACGATACAATGTCAGAAGGATAAAATCACTATGGCCTCACTCG |
| | | GTGCATTTGATTTCAAAGGCTTAATGTAGCTCTGTTCGCACTCGTGGATATAGTTG |
| | | GAGCCAGATAGACTAGGAAGATGTTTGTTTAGATAGTATCCTCGTTCGTGCATAAT |
| | | ATCCTTGAGATAGTATAGGTCGAATCTCCACAGCAGCAAGATTCTCCGTGAGCATT |
| | | GCCACTCTTTCAGTAGTAAGCCTAAGTAATTCATTAAGCGTAATTAGAGACTTATT |
| | | TTCCATATCTGCGCGTCGAGTTTCTTCTGCAGCCCTAGTTAGGAGACATACGGGAC |
| | | GCTTGCGTTTTTATCGTAGATTCACTTAGTACAGGGAAGATAAACATGAGAGGAAA |
| | | TCCGACACCTAACAATACTTTCAAACTGAGGGGCTGGATTGTACTTACCTTCACAT |
| | | CATCGAAGTCAATTCTTCACCTTCACAAGCTCTTTCTTCG |
| 51 | 43.30% | ATTTACACCCATGCCGAACATAAATAAACAAACACAAAAGGATGAGAGGAATAATG |
| | | GGTTAACTAAGGGGAGTCGAATCGTATTGATACTTATGAATGGCTATGTTACACTC |
| | | AGGTTGTACTGGATTTCGTTTGCGCTACAGCTTAGACCTTTCGCTAAAGATACACG |
| | | CCGCAGTGTCTGAAACAGACGCACATTTAAACCGCTGGGCTGTTAACGCTCATTCT |
| | | CGCTGAACTAGTCTGTCATTTATCAGTGACATCAGCTTATCTCCAATCCTCATAAG |
| | | ACCGTCGACAGGAACCCTCAATTCCACTCGTAACAGTCCCACGCTGGGTTGCGTAG |
| | | TCTGTTGTAAGAATTCATTCATGGTTGAAATGGGGCTGATGACTATGAGGCGGCAT |
| | | CTATTGGTATGGTTTAGTAGACGATCAGAGGAAGTCTGTATAGTCAGGGCTCAATA |
| | | TGTATCCACGTAGTAATGTTGCCTGCTACCGACACGATTTAGACAACGTCAGCGTA |
| | | ATTACGAACACGACCTCGGTTCCACGTGTCATCGTCTAGATGGTCCCTTTGTTCGT |
| | | AGGCCTCCAAGACCTCAGTAATATCTAATTCGAGCTTCAAGTTTGCTAGACGTTGA |
| | | CTTGACGTAGCAGATAAATCGCACTGTAATGGAATGATACCTGAATCCCGTTAACT |
| | | TCCAGCATGGCACATACGATTTTTAAATTACGCTTAAGATAAAGAAGCAGTGCGGT |
| | | CTAATCCAAAGTGCACAAGCATATCAAAACTCAGGTCTGGTTTGTACGATTATTTG |
| | | GAGCAGATTTTCAAGATAGTTATGCCAATCTCTCCATAACCATATACAGTGACGGG |
| | | GACCCTCTATGATACGTCATCTCCGGGACCTACTTTGACGCTGGAGTCTTACAGAT |
| | | GGTGGGACCATTTGTGCTTAAGCTACTTTTAGTGCGGTAGGAGCCCTCCACAATAT |
| | | GATTCAAACCTAAAGAAGCTAGGAGCCCTCTCGACCCTGGTACTTGGCATTGGCTT |
| | | AAATTTCACGTATACGCCATAGCAGATTAGTTTAATCTCCGATTTTCAAAATACTA |
| | | GATAGGGAGAGTTCTATACCACATTAACTCGCCCCGATGGGAGAACGCACAAGAGT |
| | | TAGTTTTCGACGCCGCGTAAAACAATTCAACATGGCCCTCGAGTCTGCTACTGTAG |
| | | TGCATGAAAGCTTTCCTAGTTGGGCTAGTAGCCCAAGATTCTGGAAAAATTCAAGT |
| | | TAGTCGACAGATGTTTCCGCCTTACGAGTAATTTAAAGAGGTTACCCCGAGACCGC |
| | | AAAGAGTTTAGTGCATCTTATGTGCATTGTGTTGTTCGTCAGGGGGCTTTGCACCT |
| | | AAACGGTCTTACGTACAAGCTCAGTTCGTGGATACATGAAAGTCTTGGAGTCAAGA |
| | | CCTACAAATCGACGCGATTCTAAGTCTAATGTATCCTTACTTCGGGCGTATTGTGA |
| | | TAGTATCATAACGGTTAAGACAGTTTAGGATAAACCGCAGAGACAAAAAATCTCGT |
| | | TCGTGTAACTGAGTATATAGTGTACACTTGTGCCCGCAAATGCATATTATTGATCG |
| | | AGTAATTTAACGTGTGCCTCCTTGGTAGAGGGTTTCCCTAACATACTCCTTTTCCT |
| | | GATTACCTCAGTCTCCTGCTTCAACCGGTCTCCATAAGTGAGAGGTTGTGTGTACC |
| | | GCACTTTAGAAGAGTAGAGGTTTGGCAAATTTTGGGAGCATTAGACTAGTCGAATT |
| | | TCATACTTCTTAGTCGTCTGGGAGAACGTAAGACCTGATTAAACGCATGATACACG |
| | | AAGTCATTCAGTTCTTCAGTTAAGAGGTTGCATCAAATAGCACTAGCTTAAATGTA |
| | | AATCGTCTTAAGTCCAACTATTATGCGGCACTTGATCACCATTTCACTCACCTCAT |
| | | CACTACGCTTGATAGTATGATCTCATCGTGATGGTACCCAGTTGAGATCAGCGAGG |
| | | ATCTCCTCATAAATTTACACATTGTTAAAAGGTCCCGCGC |
| 52 | 41.50% | TAGATCTGCTTTGTGAATGCCGAATTTCAGATTGACTGTCCGCGCGCTAGCTCATT |
| | | ATGACCCGGCAGTTGAAATCGTATAGGGTTGGACCCAACTACTAACGGAACTCAAC |
| | | CACTCGCCCTGTACGAGATCACAGGGAACGTCGGCTAAGGAGGTTATGGTGGCCTT |
| | | ACCTTAGCACTATATAAAGTGCGTTCGAAACCTCAGTGATTCCCCGATAGTATGAT |
| | | TTTTAAGTTCTAAGATTAAATTTGATACATCAGTTGGTCCTAGAGTTAGTGCTACT |
| | | AAGCTTAAATCAACCAAAATTTTACCCGTTCTATTCAGAAGGAAACTATAGTGGTA |
| | | GCAAGTGTGACAGTAGGTATAGACTTAAATAGTTACGGCGAAATAGAAAGATTACG |
| | | ACGTTCAGCCTTGTGTATCGAATTTGTGACTTTAGAGGCACACAGAGTAATGGACC |
| | | TATCATCTACGTCCTGTCAGAGTATCATGTGCATGATTCGACAGAAATCTCAATAA |
| | | TAACCCAAATCGGGCTCTCTTGCATTGAATAATTCATCATCAACATGAGGTAATAG |
| | | CAAAATGCCTTTACTTCAGTTGATTAGGGTGATGGCCGATCACCTATGTATTTGAA |
| | | CATATATTGTATATCCGGTCGGAATATGGCATCCTTAGCCGTCGTGCGCCGGCTTT |
| | | CGGAATTTGATCTGTCTCTGTTTAGACGCGTAACCTCAATTCGCCGCAAACTAGAT |
| | | CACTATTCTAATAATCTCACTAGGAATCTATTCGACATGCGATCTTTGATTATAGG |
| | | ATTCAGAATCTAAGAAATTGCTACGATGGGGTGTCATAGCGATGTCTATTTGAGTT |
| | | TCTATAGTGAATTGGCCATTTGTTTTGGCATCATAGATCGCTGACACAATCATTGT |
| | | GTCTTTCATCGATCTGGAGTACAGTTAGAAGAGAAGCGAGGGCTGGTAACATGCTT |
| | | ATAGATTCTTATACTTACTACCTTAGGGTACACTAACAATATTTGACATTATAGGT |
| | | CGACCAAAAAGATTTCTCTATCAGGTTTAGAGACAAAGTCGTCGACATATTTCTGT |
| | | TTGAACTCTTGAGGATGCACGAAAGTGTCTATCGGGGTATCAGTGAGAAGGCGTGG |
| | | CAAGCATTCTCTAGGTGAATTCCACCCTTTTTAGTCCTCGTTAGTACCCCGTAGAC |
| | | CGCGGAACATCGAGAAGTTATTCGTAAACGTGTCTATCTGTTCTATGTTAGGAGTA |
| | | GGTCATTGAACAAATTGAGCTTTCAAATAGATTCTAGAATGTAGCGCGTAAGTATG |
| | | TCCCGATAGCGGTTTTCAGTGTATTAGTTGCATCTAATGTAATTGAGATGAAGAAA |
| | | ACCTTGGTCGAAGAGACATGCCTAAAGAAGAAGGCTAAGTGAAGGCCTTTATATCA |
| | | CGTGGTTCATAGCCCATTATATAAAAATTTATATTGGAGATGTCCCATTGGTATTG |
| | | ATAGATGGTTGGTAGCTGTCAGCAGTGCGCCCTAGGTAAACCAGAAGACTCCTTAA |
| | | CAGATCGGTATAATTATTCGAGGTTTCCGGCTCTAGCATTCAGACATGGAAGGTTC |
| | | TTTCTAAGCGGATATATTGCTCGAAGCCCGTGAACCTTTAGAATCAACCTTTATTA |
| | | TCTCTAACCATCTTTTTTACGTTTCACCTTTAACTTACGCGAATCGATTCACGACT |
| | | GCCGAAGTACAAACGATGACTCAGTGTTGGTTTTCGCTACAACATTGAGCTCAGCT |
| | | CTATAGCGCGGACTACAAGTTCTGCGTAGATTTTGCCAAAAAAAGTTGCGGGTAGC |
| | | CTTATTCATTTAACGTATGACTGGGAGGCGCTCAAATCTCTCACTGCACCTATTCG |
| | | CAGACGCAAATTATGGCGTCGACCCCAAACTTTCAGGTAAATAGCTCACAAGATTG |
| | | ACCATTGGCAAGTTTGAACTAGTGTCGTAACGTCCTGAACAAATGTTTTTCTAGCC |
| | | GCTCCTGCTAACCTTATGGACATTTTCCTCTTCACCCCTG |
| 53 | 39.40% | AAACTACAGAAGAACCCAAAGGCTACTCACTCCCTTTGCTGTGTTCAGCTCGCTGG |
| | | CTCGTCAAGATAACGGACTCATGTCTGTGGGCAAAGCAATTTATTACAGCTATACC |
| | | TTTGTGGAAAAGTCTCCTTGTAAAATTGTTAGCAATATTGTTTCGAGTTATATCGA |
| | | ATTTAAGGTTTATTGTTATTCGTGACCATAAGGAGCTAACATGATGCGGTTTAATG |
| | | CGTATGGAAAAGCGATAGTGTTTTTAGTGAGGGAATGTAGAAGACCTCGTTTCAAC |
| | | CCTTACCATACCCGAGGGTGTCTTAATCTGTTATTAAATAAAGAGCAGCAAAATAA |
| | | AAAAAAAATGCAGTGTCTATCAAATTCCCAAATTTGGCTACGTCGTTCACTACCAA |
| | | TTTTCAAAATAATAAGAAGAAGTATATGGATCCAGTCTGATTGTCTTTCCGATCAG |
| | | CAATATAAAGCACCAACGTCTTATAAGAGCTAAATAGTGATGATTCCATGCAGTAT |
| | | AATTCAATTCCCCTAAAGCTACTGTCGATAAACTTCATATAACATATGTACTTGGA |
| | | CCGTTTGGTTTGGACTTGACAGGCTTTAAGCAGTCTGCATCATGAGCCTCCTTCTA |
| | | GATGTGCAAGCATTCCCCAGAGGCGGTTCGCTTCAGCGTGGTAAGGAATGATCTCT |
| | | GGGTCGGAGGTAGTGCAGAATGACCACTTATCCTATCTAGTGGTTTACTTTATCTA |
| | | AAACAACAGGGGACTAGATCTTATTATACGGCCAAAACTGAAATGAAGATCATCTC |
| | | ATGAATATTCTCTTAACATGAGAAATTTCCGTTGTCAATTTTTAAATGGATTAATG |
| | | TCATAAAATCTGGGATATGGCGAGCTTAACACAATGCCCCTAGTTTACGTTAAGAA |
| | | ACATTTGATACATCAACAAAACGTAGGATCCGCCCCGGTTTTTTGGAATCCACTTC |
| | | TAGAAGCAGGAGCGGGTCGCTGTATTTAAGTCATAAAGGACGTCGTTTTACGAACA |
| | | AGACCGTGTATGAATCTGGACTGTTACAACGGCCCATCCCCACCACTAGTTATACT |
| | | AGTCACCGAATAATCTGAACTATTTTACTAGAAAGTCTAGAAATTCATCCTTTGAC |
| | | ATAAATGGATTGGAATTAAAAAAAGAATTTCAAATATAATCATATAAAAGTGGATG |
| | | CACCAGAGCTCATGCGACGTCATTCTACGAGCGATTTATAGCTTATACCAATAAAC |
| | | CCCGCGTGTATTAACGGTCCAGTCAAAAATACTATGATACCGAACAAGGTTTATCG |
| | | ACTTGTCCCGTTGAAATCCTAGATGAAGTTTATAACCAAATGGCGCCCCTTTAGTG |
| | | ACGCTGTAAACGCAGATTTATCAAACAGGAAACATTTCTGATTAACCAGAAGTATG |
| | | CGTAGTGAAGGTATATCGCGCAGTAACATTCAGGTGCTTCGGGGATTCAAAAACGT |
| | | GTTGCTGGTATAGCTCGCCTGTTTTATCGAATGTAGTCTCAAAATCTAGCCGAGTT |
| | | TATCAACTGGTCGACGCTGGAAGTCTGCACTTGAACATCGTTCACATGTAAGCCAG |
| | | AGATAATGGCCTCAGCATCGTCTTATTGCTAATCTCACGCTGCTTTGTCGCGACGT |
| | | ACTCTCTGCATTACCAAATGGGATTAGTTTAATTTCGTTCTCTGGGTGACCTTGTG |
| | | CACGCTATGTGGGTTTGTATTAGTTGATTAAAGAGTCCCTTTGAAGATGGCTTCAC |
| | | TCACCACATGACTACACTTCCTATCGAGGTAAGGAAACGTTTTCTTGTGCAAACAC |
| | | CCCAGACTTACCAAGTTTAAAGTTTTGTATAATATTAAGAATTTATCTAACACTGA |
| | | GACACCATACACAGCTTCCGTACCCTATTGGTCCACAATATAAGACGTTAGATATT |
| | | GCCAATAAATGCTTCATTCGGTTTTTTGTTAGACAATTGGAAAATCTTATACATAA |
| | | CATATAAACGTTTCGCATCCCTGGTTCCTTCCGATAGGTC |
| 54 | 40.50% | TCGTTTTATCACGTTTTAACATTGAATCTTTAGTGCAACCAAGAGCCACTTCTCCT |
| | | GGGTTATAATCATCATCTATTTAGCATACCAACGCGTTTGGCTGCCTCGGTTTGTA |
| | | TATAGTCGTAAAAGCCTCCGGTTTATGAGGTGATGGAAATTAGTTGGATACTTGAA |
| | | TAGATAATATCCCATGCGGTATTCACCCACTGAATCACATCGCCTGATGATCCTTG |
| | | CTGTTTGCGGGAGAGCTCTTCTAATGATTTTTGCAAATGCTGTGCATCCCTAATAG |
| | | TCTTTTACAGGGCAAAGTACAGGGATTGACAGCCCCCGAATGTCTACAGCCGACAA |
| | | ACCGAAAGTCTTCTACCCCGAGGTAGCTGAAGGTGCATAGACGTAGACATGTTGAC |
| | | TAATCTCATCTTGTCTACTATCTTGTACACAAAATCAAAATTACAATTATATGGAA |
| | | GGCATGGGATGAGTGATCGTTAATTAGACAGGGGCGTCTTTGGCAATGCATTCTCT |
| | | TATGATAAAAGGTTGACCAGATTACTGCTCATGACTTAGTGTCCACCGGCCCAACA |
| | | ATTAATAATTAAGAGACTCAACCGACATACGTTAATACCCAATAATGCCCCAATAC |
| | | CCAGACTTTTACAGGGTTATTCGTGAACATGAGTCCCTCGACATCTTCCCAGATTT |
| | | TAATCCCCATATTACTAGTTTGTAACAGATTGGTTATGGGACTGATTAGAACAGGG |
| | | AATTTCAGCTGGAAATCACTACTAACTTATTGCTAGTTTGCCGATCTAAGAAGAGT |
| | | CTTTGCTAATTGATTTTAAAGAGATATTCTGAACACGTCAATATCCAAATTTTATC |
| | | CGCACCATTCTGACGTAATGACGCCTAGAGAACGAGTTGGTGGCAGTCTATCGCTT |
| | | CTGTTTATTTTAACCTTCAAAATATGATAAGGCCCCAGTTATAAACTATTTTTTAC |
| | | GGCAACTTCGGATTAAGTGTTCTATACGCCAAAACTATTGATTTACTTAACATTTC |
| | | ATCCCGAGAAGCTCCGTCTTATCAAGTACGAGATGATCCCCTATTAGAAAAACCAC |
| | | GGCTAGTATCAACGACATGCGTTACACACACGCCTCAGTGGGGGCCGTCACACATA |
| | | GTTCAAATATTGATACTGCTCGTCTCGATATGTGTTCAATGTCGGCAATCAAGCAG |
| | | TGTCGGAACTGAACCCGCACTACGGGCTCGTAAACGACCCAAAATCCCCTAATCAA |
| | | TCATTGTAGTAATGGTAGCAACTTGTATGTCCTGTCAACGCAACACCCTCCTGGTG |
| | | AATTATTCTATTAGAACTACTAAAAAATAAACCCGAGGTCCAGCTCTATCGTACAC |
| | | GACACGAAAACGTATCAAGGTACAGTTCGATAGCCGTACTTATTATGGTGACTAGC |
| | | GCCATATACAAGGTCATAAGGGACCTTGTTAGCGGTGTGTTCACTTCATCGTCAGC |
| | | GACTCGTTCGACTGTCATTTCAATGAAATCTTTAATGAGTTTAATAGAGTAGGAAG |
| | | GGACAGTAAGATATTTTATGAATAATGTCGTACGTAGGATTTTTTTCAAATGATGA |
| | | CTATCACAGTACGGCATACGGAAAATTCAGTAGGGAATTAGATCAAGTGTAAAATT |
| | | ACTGGTATACTAGCGTATACCTAGTACGATGATAATTAACAATCACCCCCAGCATG |
| | | ATGTGAGAATAGTAAAGTATCCATATTTACAACTAAAAAGCTCGGAAGCTGAAATC |
| | | CCAAACCGCTTGAACAGCTCTCGAATAATACCGGTGTTTATCATCGGAAGGACAGC |
| | | GCCTCAGGATTTTCGGCAAATCATAGCTCTTATCTTCGATCTAAGCGTTTGATGAA |
| | | TATTAGAATCGGACTGAGATATAAAGAATAGTGATATATGTCGGAAAACGACGATG |
| | | TCATTTTAGACTATGATCTTAAGACGGAGAAAGCTACCATCATAACACCGACTTGT |
| | | CCTGCCATTGTATTACTGGCTTTCCATCGTGAGGGATAGC |
| 55 | 42.10% | ATTATGATCCCAGGCTTCGTTGAGTCTAATAGCTATCCGACTAATCAACTTCTCAG |
| | | GCATGTCTCGACTCCGATCCTGGTGGCCTTAAATTTCTTAGGTGCACGGAATTGTG |
| | | TGTACCTGGTATGTAGAGACTATAACGACTCACTTCTTGCCAATTAGGATTCAAAA |
| | | CTCCCTACTTGAGCAACGTGTTCCCCCGCATTATCCATATCACAACAGTTGAATTT |
| | | TTCTAACGTCTTCTCCTCAAACCGGAGGGAAGTGTGAATGTACTGTTGTCCGGCCA |
| | | TGCCTGAGGTATTTTGATTCTAGTTAGTAATTACATTAGGAACTCACTTCGTCAAC |
| | | TCAAACACGTTGACAAATGTGCAGTTGGGTAATACATGCCGTGCAAAGCATGTATG |
| | | ACCGTGGTCTACTAGATGGCTTCGCGATTTACTGTTTTGCGATATAGGCGTCGGAA |
| | | TAAACTTCAGCAGGTGCGGATGCTGATCTGGCGCCGTCATTTATAAAGATATGGCT |
| | | ACGACTTAGCTCGTGAGATCGAGACAAAATCAAGATCTTATCGTCTTCCACAAAAA |
| | | GTACCCTCAATCGGATATTCGGACCGTAAAAAAGAGCATGGCGCTTGATTATCGTA |
| | | GCTAGCGCCCAAGGAACAATTGTATTATTCAGATTAAACCCCGGATTGGACCTATT |
| | | TTCATCCTAGTAGAAACGGTGACGACGCGACTTCCGAAAACTCCAGGAACAGTGCG |
| | | GTCTACCCAGGTTGTAGTAGATGCCCGTTTTCTCAGGGCAACCAGGGCATCATACG |
| | | TTAACTTAATCGGTTTTAACCGCGAAGTTCGATACGGACTGATTTAATAATAAACG |
| | | CGAACAACCTAGTAATATCATAAATTGCGGCGTGTACTTCAGAAATGGTAACTAAA |
| | | TGTCAGACTTCTTGAAAAGGAACAAGCGCGCTTTCTCAAGTTTGTTGAGTCTCATC |
| | | ATAATGGGGGAACTCCGTACATGGTCCGATGGACTCGATATCCGAAGGCGATAATA |
| | | ATTATCCCCGTGTTCTACGCTATTTACGAACTATTAATAATGATCGGTCATGTCGG |
| | | TGGTTTATTCCATTCCTTTATCTCCGATAAGTACGTTACCATGGGATTACGCAACA |
| | | GCTAGATTTTCAAATGATCGGGTCGAATCCGGCCTAAACGAAACGTCGCTAGCGAT |
| | | TGAGAACGGATGTACAGATCTCTCGAATACATGAGATGCGCGTAATCATAGTGTAC |
| | | GATAGAACCTCATGTTATCAACAGGTGCTATCTTAGTAAAATACATAGTCATATTC |
| | | TTTACACGCGTAAAGATTCTTTGAGCCAGCGAACATGGAAATGGGCGTTGGTGTGT |
| | | TTCTCCCCGGCTTTCGTAATAGTCGCCACCATCCGCTTGGGTGCTGATTCGATCAG |
| | | TTCTAACCAAGGAGCCTGACAGTCTTCGATTTTTGTGTATTCCTGTAGAATATGGC |
| | | ACCATAATTCAGCGGGAAAAAATTGTCAACTCAGCAGTGTCTATTAAGAGATTACT |
| | | CTCGCTTTTGGACTGGTACAGCCTTTACCTAGTAATATAGACGGACAAAAATTTTG |
| | | TGAGTCAGACGGCATATCCTGAAAACAAATACAAGTGTAGTCTACGTTTTAGAATA |
| | | GACTGAGTGGCGTCGGTAGAAGTTACTGCTCGAGTTATTGTAAAATTCTTGCCAAG |
| | | AACGAAGTTACTCCATATGGAAAAGATGACTCAATCGAGTCTTACTAGATTATTTC |
| | | CGAAGTCTTAAACGTTTAGACCTAACTTAGTCGAAAGTTGAGCTCCAGAAGTCATC |
| | | TCTCCCAGTTTATCAATAGTGGGTGGAACAAATTCATCGGCTGTTGACCTTATTGC |
| | | ATCCACCTCGTTGGAGTTATCTTGCCATGTATCCTCAAGTGTTCCGACCTGGAAGT |
| | | ATGTAGAAACCCCTTTGAAATATCTATCACAAAGCAATATCTTATATTATCTTCGT |
| | | AGTTTTTAGAATTATATCTATTTAAGGGCACAAAGTCTAG |
| 56 | 41.70% | TTAACAATAAATGATTAGGTTGTGCTTGCCTCCTAATTTTGTTTAAAAAGTTGTTC |
| | | TTCTGCTGACTAGTTTGATTCTACTCATTTCTGTAGTACCGGTTCGGCGTACTTTT |
| | | TTTAGAGGAAAATACTAATGTGCGGAGGAGGGCTTAAGAAAACTGCAGATCACTGG |
| | | ATGAGCAGGAAAACCGAAGGACGTGCACGAAAATCGGACTTGCTGTTGTGACTATA |
| | | CGCAGGCTAGAATCAATACCGTCGGTGCTCGTGCCTCAGCCGTATCAGATATGATT |
| | | CTTGAGCGATGTTATCGTTGGATCAAATAGTTCTTTTCGTGGAAAGGTATGGTTAG |
| | | ATATCCGGGGCCTCTTAATATTGGTTTCGACTAGATCTGACAGAGTCGGGTCAAAG |
| | | CTAACGCTGTCGCTAATGATGACAGTGTCAATCTGGTTAAGTATACTCTGGAGTTA |
| | | TTAGTCGATCTCTCTCAGTGTTTCTTAAGGTGTTCTCAGCTGGCCGGGTTGTGCGC |
| | | TTGTGAGGGAGCGATAGCAGTTTGTGCTCGGTCTACGCAGTAGATCGTTCACAACT |
| | | TAGTCAGACCAATTTATATTCCTATGCCTAAGAAATAGTAGATCATCTAAATGTAG |
| | | TTGCCGATCAACTCAAAAATCATGAGCAGTGATAAACGCTAGTACGGAGCTAGCAT |
| | | ATGCGCCTGCCGATAGATTGCATAGAACCACAGAATCTCTAAATTTCTGGCACTGA |
| | | CTTTACCTTACTTGTCTACTGATCATTTAGTTCTAAGGCGGGTCCCAGCATATACT |
| | | GAGTAAAGGAAATTGCAACGGTCCAACAAAGAATCAATAAGTAAATAGAACTCATC |
| | | AATCTCCATGGTTTTTTACCCTGTGGTATGAGAGCTTCGAGACAGTACAAATACAT |
| | | TCTACGAGTGCATTTATTAAACACACGGACCCTATACAAATTAATAGCATCACTAG |
| | | CTCGAAACCTATTACAGCCTGAACGTTTCGAACGCACTTCGGTATACAGTGTACTC |
| | | GCGCGCGTGTTGAACCGAAGGTGCTAGCCGAATTAGTTGGATTCGTATATATGTGG |
| | | GATCCCGATTTCCAAGTCCTTGCTGGTTTAACACACGGATATTAGTTGCTATTATT |
| | | AGCGTGTTTGAAAACCATGTCAGAGTTAACGACCGGCTAAAAAGCCGACTTATAAA |
| | | AAGCCGAGTGGTTTGGCAACCTTCTACTGGTCTTGGAATTAACTTCTGAATAAATA |
| | | CAAACATGAAAAGAGTGAACTGCTAGACTGCACCTGTGGAATGATCCATAACAGTT |
| | | AAATTACTCCGCCGAGTCCATTTTGCTGACGGTGGATTATCCTAACTGAAGAGCGT |
| | | ACAGCGATTCTGTCCAACCGTTGAAATCAGTAATTTTCTATACCTACTATCGTTTG |
| | | ACCAAACTCAGGGAAGCATACCTAAATATCATCAAGGCGAGAAACTTTTAGACCCA |
| | | TAGTTGTATTATAGTCTAATTTCAATGCACATTCTGTTCAGGCACAGACTGATATT |
| | | GAAAGAGGCCCGCGACTTTGAAGGTGGGCTAAATTTATGCAATAATGGCACACCAA |
| | | TCAACACAGTCTAGAACTTACCAAACCAAGCCTAGATTCACCTATCTATTTTTGAT |
| | | CCGACTGTATAACGTATTGTAATACCTCAAGACATAAGACACTCATAACAATTTAA |
| | | CTTTCTCTTATTAGGAGGCTCCTCTATGGGATTCGTCGTCGAGTTAAATGATTTGA |
| | | GGTTTTATGTGGACTCCGAGCACGCCCGGTAAGAATTTCTAGGACTTAGGATACAA |
| | | TGCAACTCAGTGGAGTATGTTCCCCCGTGTGATCTATATGATAGCTGAGTACGACA |
| | | ATAGGCATGCGATTCAGACTATCCGCTTTTAATTACCAATGAATGTCACGACGGAG |
| | | AACGTTATGAAAGGTTTTCTCTAGCACGCCCTATCGCTCTTATATGCGAAATACAT |
| | | TCCTGCTTGTGAATGGCCGGGATTGCTTACACATTAGCCT |
| 57 | 38.40% | CTTAAGATTTCAGCTAGAATGGTTCTGGCGCGCCTAAGAAACTAGGTTAAGTCTTC |
| | | TTTTGCGCGTTAAATAAAAATTTTGTCGGTAGTTCTTAAATGGTGCACGAAGTTGA |
| | | CTGCATATATATATGAAGCACCTAAGAGCTCTATCCCCCCTTAAATGTCAAGATTG |
| | | GCTAATATACCACCCCATACACATGATTAACCCGGTTACCTTCGACAGGTTTGGAT |
| | | CTTTAAATACAATTAGTTGATCTTCGCTCTGGCAGAGCTCGGGTTCGTTCGTAGTG |
| | | TATAAAATATCTCTACTTGCAATTATCGTTTAACCCCTGCAAGAGCGTCTATTGGT |
| | | CTTGCTGTTTTCTTACAGTTGTATGCTCGCCATGTATAGGCAGGTAAACAGACTTT |
| | | GACAAGGGTGGGCGAGTCGCGTAGAACCTTTCCATGAAGGCATTTATTTTTGATTA |
| | | TCTCTGATACCTGGGTGTGTATAATTGGATGCAACGTCGCTTGCTAAGACATTCGA |
| | | GCTCGAAATTCTAGGATTTTGTCTATACCCTTTAGAATCTTCACTTCTATAAATGA |
| | | CTAAAAACATGGGAAATGACAAATTAGCAAGCGGCGCTTTTTTGAATCAATCACTA |
| | | GATATATTTCTAAAACTTAGCAATGCTTTCATGAAAACCACTAATTTTAATTACAT |
| | | ATTTGTAAATAACCCGCATCAAACGCAAGTTGATGTCGCATCATATATATCTCCAT |
| | | AGTCATTTCTATTCAACTGGCATGTTCGGTTAATCAAACAAACCTGACAACATTAT |
| | | TGGTCTCATCAAAATTTGCTCTATTGGCATCCAGAAGATTGAATTTTGAGTGACCA |
| | | GTAATATTACCCTCTGGGACTACTTGTATCTTTTGTAAAAGACGTATAATTGTAGG |
| | | GAAAATTTGAAGTTGTAAACTAGAACAATGAAATAAATCACAAGCCTCTTAAATTT |
| | | CCGAGTGTGTTTAATAGCTGTCCGAAGAATAAATATCCAGGGAGGATCTGATCTCT |
| | | AAAAAGGAAACTTTCCTAGGTGCAATTCATGGGACAATAGTCTTTACCATCATTTG |
| | | GATCGGAATCTTTAAAGATTTAACGTAAAACTGTAGATGGGTGAAGCAACCACTGG |
| | | TGTCAGGATTGTTGTAATAACCTACAATACGAAAACACATGGAAATATTTTTTTCA |
| | | CGAGCTATACACGTAGTTATACGTATGAAAACAAACAGGACTCAAATAATCTATAG |
| | | AGGAATTTATAGGTTCTTCGTGAACGTTTCGAGAGCATAGACATGATTACAGGCTG |
| | | CAGATGATTGCTCTAGGGACACTGGATACGTCTGTCTCAGTATATTAAGAGGCATT |
| | | AACTTATAGAGCTGGTTTGAGTTCCTCATGAGAGAGAATATATATTTGCACAATGA |
| | | TACTCAAAAACTTACCGCTCTGCACAATCCGCACATCGCGATCATACGCGCCGTTA |
| | | AAGTTATCATCCAATATACTCATAAATGGTGTAACCTAGCTCCTACCACAAACTGA |
| | | GTACCGGGATCGCTATCCACATCGCTGAAACAATGGGAAAAGAAAGGTTTCCTTCG |
| | | AGTCACGCACTGACTAGATCTACAATACTTATGCTCTAGAACGCGTGATATTTCTA |
| | | TGTAAAGTAAAGCATGCTACTAAGGTACATCTAATTTTACGAAACCGTATACTACT |
| | | ACTCGCCATTGGTATACTTTAGACTTTGTAAGTAAAAAACGAGTAGGGCCTCAAGG |
| | | ACATAGTCACTGCTTATACAGCGAAACGAAGCTGCTAACAAAGCTCAGACCGGTAT |
| | | TGCTGTTAGTATATTCTTGTTAGAAGCGTACATCGGTTGGGCCGTATGGTCCGATT |
| | | ACCTTAAGAATAGTTGACTAGGATCGTCTCTAAGGTCGTACTTACCCACCTAGCAG |
| | | CTGATATCTTCGATGCCTATATCTGTATAGGTAGAGATTCATTCTCAGCGCATTGC |
| | | CGCGGTAGATCCTATGTAGATTATTTAGCATAGTTAATTA |
| 58 | 39.10% | GAACCTTGGGTCCTTATCCTGAAATAAAAAGAAAGTGCACGTCTCCGTAATATATG |
| | | GATGTCTCAGTGATATCCACGATTACATCAAGCTGAGTTATTTTTAATGATAGTTG |
| | | ACTGTATTGCCTAAAACGTATCTGTAGTAATGAATACATAAAGGTACTGGTGATTG |
| | | AGAAGTTCTCATTAAACGTTAAAATCCGCATCATCTGTAAAAGGTGGGTAATTGCA |
| | | CTATAGAGGGTAGACCACGCCTGTAGCCCGCTTAGAACAATTCTTGTACTATCATT |
| | | TTTAAGTCCTTCAATGTCTATCATAAGTATTGGACATTGCACGAGAAAACACGGGA |
| | | CAAAATGCTCGTCGTTTGAGACTATGGATCGCTATTCGGGTCGAGCAATCTGAAAC |
| | | AGATATTGTCATGTTTGGAAGGTGAGCCCATTAGTAGTAAGCGCTTTATACCACTA |
| | | TTCAGGAGTAATAATTTAAGGAGTGTAACAGTATGATGTCTACCGGTACACGGGAG |
| | | ATTGTAATACAGTAGTAGCTCCTTATGGCTTGGGAATAAATTACAAACTGAACGCT |
| | | TTCTTTAGAGCTCTAGTGTCCTGATTTATGGGTAAGGCGTATTATCTGCAAGTCTC |
| | | AGTTCGGGATAGGTATTCCGTCATCTAATATTACCTCTAGGGTGTATACTACCATC |
| | | CTTTGCAGACTATAAATACTATCTATCGTCGGCACTGATAGATGGAGGATTCCTTG |
| | | CAAGACCTGATATCTCCGTCTCCATGTCTAGTTTATAGATTTGCCTTACAAGTTCA |
| | | TTTATGCATGTGTAATAGAATGATTTATATGAACCGTCATAGTTCCATTTTAGCAT |
| | | CCGAGCGTGTGTCCTCTCTCGTAATTAGGCGTACGTCGAATCATTTTGCTTTCACT |
| | | GTAAATAGGCAAAGCAAAATGTAGCAAAGGAAGGAATGAAATGATCATTCTCATGC |
| | | TACATGTGTCCTTATACATAAAAATATATATACTTGATTAATTGCACATGAATCAC |
| | | TTACATTCGATTATCATAATACATCCCCCACTCGGATTGCTCCACGACCAGATGGT |
| | | TAAAAAGTTGAATCTGTGCTTTGATTTTTAAGTGAGCACTCACGTAGTATGAAACC |
| | | GCTAGCTCAGGTTTTTTTTGGGGATCGTTCAGTATTCACGAAAGAAGAATGCGGCG |
| | | GGGTGGTTCCACACCATATCAACTAGTGTTTATAGTTGCTTATATAACGGCAACCG |
| | | GCTAGTAAATGGTAACTTAACAGTAAAATGTCTAGGATTAGTAAACATATATTATG |
| | | GAGGCGTTAAGGCTGTACGCCTTGATAGTACACACCTTTTTACAATCACAATCCTA |
| | | GGTTGATCTAAAACCGTTGACGTCAAGTCCATTATAAAATCTTAATCGCCTGATTT |
| | | CCCTGTCCTAAAATGAAGAGATTAAAGAAGTGAAATATATCCCTAAGCCAGAAGTG |
| | | GGAGAATACCATTTGGATATATGCGAGCTTCTGCCAAATCTTAGAGATTTCTGGAC |
| | | TTTTCAATTATCCAATATGAGGCTTGAGGATTACCAACTCTGGACTACATGACAGT |
| | | TCCACAGAAACTATTTAGTTAGACGCAGAGCCAATTAGAACCTCGACAATTAGGTA |
| | | AAGTAAAGTTTACAATACTGTTAAGTCGCGTAAAAAAGGTTGATTCAACTATGACG |
| | | GGTATAGAGGAGGAAATAGAGGCTCTCGTTAGCTGTGTCGTTGGACATAGTAACTT |
| | | TTTACAAAGAATGTTAGAGCTGTTGAATATTTACGCTTATACAAAGTATCTGCTGT |
| | | ATCACGACGGATTTTATCCATGCAGGGCAGTAATCCATCAGGCTTTTGGAGAGGAC |
| | | AGCCTTGGGAAGGATATCGTCACGAGGCGTTTCGCACTCAGACACCCGAAAAAATT |
| | | ACGAGGAAATGATAATCGTAACGTGGCGCCTAGCGCTGGATAATTACCATAATTTA |
| | | ACAGAGGCCACAACAGGTTTTCACCCTTCAATGAGTGTAA |
| 59 | 41.00% | GATTCTGTACAATTGTTTCAAAATATAGCTTAACACATTTGATGGAATAATAAGGG |
| | | TTCCAACTAGATATAGTTAGTTAGGAGTTACGGGAGTGGTGCTCGGGTACACCGAA |
| | | GCGTTTATGTCTAAGCTCTCTTCTGAGGGGGCTCAGACAGCTGGTACAATAATTCA |
| | | TCCGAGCCGCGGTGAATGCGGCATCAGGCCCCTTCTATACTTATAAAAGAGCATAT |
| | | CTAATTTATTGGCATATTCCTGCAGGCTACATAAAGTCACTCGGTCGAGGCATCCC |
| | | TATTCGGGCTAAATTTCAACACGTCTGGTTTGAATAGCGACTGTTTTTTACAGATG |
| | | GCTTGGATAACCAATCAACCTTCAAGAAGCACAGTTCTTATGTTAGGAACCGTATG |
| | | CAACCGTAGACTCCTATTTTCACTTGCGTGAGCATTCAACGAAATTGGGAAGACAG |
| | | ATGGACTTACATTAACGTATCGGACTACGATCGTAATATCCGTGATGTGAGTATTA |
| | | TAGTATACAAGAGTGAGGAGATGGAAATCATGACGGTTATCCCACGTAGCAGCACA |
| | | CGCAGATGCAGACCAGACAGATACGAATAAACTTTTTTGTACGGTTGCCCGGTAAA |
| | | CTAGCCTGGGATCCCGCGAACAAATGTTAGAATAAAAACGCGAGAGACTTGCTTTA |
| | | GTAGCTTTTCATCAGGATTCCTTGCAAAAAGTTAACACAAAGTAAGCGTGTTGTTA |
| | | GTAATGTAATGTTTGTGAGGTAACACTGTGGGTTAAGTAGTACTAATGATCTTTCT |
| | | TTGCTGTTTGACTTTCAAAATGCGTGGAGTTCAGTGGTGGCAAAGATTGTTTAAGT |
| | | CTTACGTATTGGTAGTACTCGTTAAGCTTGAAAGTTTCGATTATCTCTTTTTATTC |
| | | CGATCTGAAATGAGCTTGTTCTATCCGAAGCTGAGGTAGTCCACTTAGACCGATCT |
| | | ATCGCTAACGAGAATAATACTTATTATTTAAATCCTTTCTCATGCCAATAGAGGAG |
| | | ACTGTCATGGTAACCGGTATGCTTGTGTTCATATTAATTCTAAGATTTGCTACAGG |
| | | ATTAAGTCTAGTTCAAGTCCTATTCCAAATACCACAATCTCTAAGGCCTCACACGC |
| | | CTTAACAGAAAGGGGATTATACGCGTCGGTTGTTCGTTATGCCTTATAGTACTCAA |
| | | CCCATAAATAGATCGCACATAAGAGTATGAATCGGTTGATGAAAAAGTACATAACT |
| | | CACTACAGTGCCGGATGAGAGATTCCCGTGAATTAACTAGTGGCTACAAAACGTAA |
| | | CGTGCGAAGAGCAAAGGTGGCCGCGATATTACCTTTACTTTCGGTGCCTTAGTAAA |
| | | AGAGGATAATGGCAAAATGAACGTCCTGGGCAATCAGACCAGAGGGAATATGCTTA |
| | | GCTATTGGCTTTGTAATTGTTGTAGTTTTTAATGGTTCTAAATATCAACAAATACC |
| | | ATCATGATAGTTACCGATCAGATGAGCTTGAGCCGTTGAAAAGAATGCAAATACAA |
| | | AATCTTGTTCATTAATCCGATGCAACGTGCCGGCTTGAAATTCATTTTCGAAGTAG |
| | | TGCGTCCCCGCGTATAGACGCTACAGTAGCTCCGAAGGTCTATTGTTAGAACAACA |
| | | TTTTAGAAACGGGCCTAATAGGAGTTCCTCGGGAAAAAGAGGAAGGGACAAGTTGA |
| | | TTGTCTATTAAGATAGATGATCCTATTATAGCGATGTCAATACTACGCCCAGTGAC |
| | | ACCATCAAAATAGACTGGAAATGATGGTACGATTGGATGAGAAGATCATTAGCTGC |
| | | CTTTACCTTCGACGACTTCGTCGTAGTGAGGGTTCTGACCAATGTCCATAGCAGTT |
| | | GAAAGCGCGACATTACTCGAACAACGCTGTGGTCACTCTTTAATGATTCGTATAAT |
| | | GAATCTTCCTCTGCAACAGTTGGACAGAAAAGTGGCTTCTTGCTTAGGACCTAGCT |
| | | AGACTTTGTTGCCTTTCTATGTAATACGTACGCAAATTCC |
| 60 | 41.40% | CAGTAGATGAGGATAAGCCCAAGTATCGATTCCAGGAAGCCGCCATATGGAGATAT |
| | | AGAGGTATCTCTGGCTTCGCGAACTCACAAAGGAGTGTCTCGATGGACCTCCATAG |
| | | GTAACAAAGATCAAGGCCCCTTACCAACTCATGTTCTATAAACTGACATCTATGCA |
| | | ATAAAGTTAACACCAGAAGGTGGGTCAGACCACAAACCACAACCCCGCTCAATTTT |
| | | AGAACAAAGTCTACTAAGAGGTGCGAATCAAGCCGAAAACGGGAGTTTATTGTCCA |
| | | TATGATGCTGGATCGGATTATTGTATTATAATAGCCTAAGATCGTGTCTCCGATCC |
| | | AAATGCGTGTACGCATCAATCCTGAGAGATCCGGGATGGTTGCTGGGGTTAATAAC |
| | | TTCTCCTTTATATCCGGATGACTGCTAATTCCTCAAATGCAATCATTCTGGAATTA |
| | | TGAGGCCTATTAAACGAATTTAACAGTACCTAGTCGGTAGAAACAATTCTACCCCG |
| | | CATCCTTAAGTCTACTTTCAGAGCTACTGGCGCCTTTGACGCATAGGTAAAACCGG |
| | | CGACTAGAGGAATGTCGTATCAAGATAAGCCCTAATTTACTTATGCTAGCCTGTGT |
| | | TCGATAAATAAGATGTCTGAATTGAATTCGCGCAGAAACCAGTGCTGCCACGGTGA |
| | | AGAGTGATCGGGGCGGCTATCAACTACGCGGTGAACTACCCCAAAACATTTAGGAC |
| | | ATGCGAATATATCAAAGAGAAATCAATTCCATTAGTTCGAAGATGAGCACGATCGT |
| | | TACTAACTGCAGACAAAGAAGGCACTATTGATAGAACCGATTGACAACCCGAACGT |
| | | GTACCGGAGTTTGGATCAGATCTTGAGACTGCGCTTAAAAGCAAGAACCCATCACA |
| | | AAAAGGCAATAGCATTAGGAGGAATCGCGCACAAGTACAATAACTTTTTCCGTATT |
| | | TTAATAATATTAATTGTCCTTCTCACCACGAGGCCGTTTCCTTCGTGGAACCAGTC |
| | | GTCCTACTTTCTCTCCGTAATTTCATTTTATTTAGAATAAAGGTATATACGGACGA |
| | | CTATCGTTCGGAACAACTAATAACAGTGCTTGGAGGTGAATAGAAGTAAGTTGAAC |
| | | TGAGCTAAAGTGAACAACTACAATTCGTAGCCCTGATTTCATTGTCATTTTTTTTC |
| | | TGACTCAACACCCCAAAGATCGCGCAAAGAATAAGGCCATAGCTCAAACCCGAAAA |
| | | AATCTTCTAAGGCCTGATAACTTAGTTATTATATGAACACCGGTAATCCCTGCATG |
| | | CAGCATATATGAAATAAAATGCCGTCGTTTTCATTGTTTCGTATAAGTAGGGAACG |
| | | AGGTCCATGTGCTATTTTGCTCTTTTATGTGTGCCCAAGGGGTACTGGAATGTCGA |
| | | GTAATACTCAGTCCTTCAATGCTCATCTTGTGACCAAATTCATTGGGGAACTCCAT |
| | | TGGGAAAGGAATCTGTGAGAGTGAATCCAGACTAGGATCTACCCACATTGTAGTCT |
| | | GAATTTTACCTTCTAGAAAGTACCGCTCAAGTTGACTATATTTTACACAATGTGGG |
| | | CTGATGGCTGGTCTCCGGTTGAGGAAGGATCAATCATACTCATCATGCATACATGA |
| | | AGATATACTAGTATGATTAACAATAGGTTTTCAAAACAGACACTCGACTTATTGAG |
| | | CACCCTATTGGCTAAGCAACTGCATCTGCACTAGCAATGGATCTTAAGGCATCATA |
| | | TAACCGGTTAGGTACTTTCTTGTTAGGTAGAACAACACGGTTGATCAGGCCAATCG |
| | | CTACTGAAGTAATGAAATCAATAAACACTGAGTCTTATGAAGTACTATTACAATCT |
| | | CCTAGGGTCGTATCAGACCTTTGTTATGTTTTAAGGACAATGCGGGATCTCTCATC |
| | | CAAAAAGCGAAATTGATACCAGGCATTGGTAGTCAAGATTACCGAATTATTTTACG |
| | | TAGGTCATTATATGCCTGCAATTTTGGCGCTTTACGCTCA |
| 61 | 38.90% | GTTTAATCTCCTTGACTAACAGGAGTCTCTTGCCAACGGATGTACGTAACCGTATG |
| | | TTAAGACATTATGAAGAGTTAATATTACATGCAACCATTCGATTTGCCATAAATGT |
| | | ACCGAACGCCGTTATATTTACTTACTGGATGAAAGATTCAAGAATCAATATAAGTT |
| | | AAAATCTTAAAAAGATCAATCATACGTATAAAGTCTATTTGCTATTAGAGACGACT |
| | | GTCTGATTTGATGATGCAGCGCGTTGTTATAAACCTCATAAATAAGAGGCGGTGGC |
| | | TTTCTTACTATTAGCACAAGTCTCACTGAGTAGTAGAATAACTCTTACTCTATATG |
| | | TTTCATCAGGTACGACCCCACGTGGCAAAATTACATTTTGCACACGAGGCACATTA |
| | | AGACCGAAGAGAACATTTGGCCGAGAGGTATGTCAAAGCCGGCTTAATGATATCGA |
| | | CACAACTCATAAATGGTGAAAGTTATAACCAGGTAATCTTATGGGATTCTGTGGAG |
| | | TAAAGCCCATTGGACTTCGGAATAAATAAGCAAGCTAATCAGTTATAATAGCATAT |
| | | ATGTTAATACCAAGCGTGGAATGAGCACATTTTGGCAGTTTAACACTAAGCTTGAT |
| | | AAAACTCGTAGAGTAGCGATTGGACACTACAAGACGCGTGTTTCGCTAGAGACGAA |
| | | CCACCTTGTGCCAACAGATTACTCTGAAGCTCGCCTATTTGTGGAAGTAAATATTA |
| | | CGTAACGGTTATAGCATTGTTAACGATGATTTTGTCGAGTAACGGTATGAATTTAT |
| | | GAAAAACGTCAAACAAGCGTGATCAGTTTCGCATGATCGAATTGAGTTTTTGCCCG |
| | | CGCAGGGTTCGCGTCAAAACACCTTAGAGTAAATACTTAAGAGGAATCGCTACGTC |
| | | TATTTGTAAAAGTCCGAGTACCCACCTTGGAATCCCCATTTTTTTTTTTCCAGTCA |
| | | GCTCAACGGTTGAATCCACGTGTCCGAAGAAGCTCTGAGCAAACTATGGTGTCGCC |
| | | GTTCTAAGCCCATTTCAAACGTTATGGAGCGTTGTGCCTCTTTGTTGGCACTTGTT |
| | | ATTCACCGCGGCGAAGTAACGCGCTCGTCAAGCGAATCATTTTATGCCTACTCGGG |
| | | CTATAGTTAACGGAGTTAAAATGCTTCAAGTGTAGGTCGACAAAAGATCAGGAATT |
| | | CGAGATAAACTCTCCATGTGAAATAGCAAGTTTACGTCCTCGTTTTTGATTATAGA |
| | | CTAAGATTACGAATTCTTTAGCGCTGGCTCATTTGAATCCAAAACCGTAGAATAAG |
| | | AACCCCAGACTTATGTCCTCGAAATTATCAGGTAAGAGAACAAATAATTCACGAGT |
| | | ACTGACAGTATAAGCGCTTATGTGAGACGACCACGTAACTACAATTTATAAACTTG |
| | | ACCGTTATTATGTAGTATTTAGTGGCTCATAAAACCAGCTTAGCTTAGATCTGTGA |
| | | GACTGACCAGCTGACCCACAAGACTTTTACATTGAAGTTGCAGCTATATGGAAACG |
| | | TACTTTATAATTTCTTAATGTAAGAATAAATTTGCTGTATCGCTTTGTTCGTTTGA |
| | | ACTCTTTTCTATGTAAAAGGCTGACTAACCCAGGAAGAGGGGAGCATATTTTACAA |
| | | ATTAGTAAGCGCTCTCTCATTCATTTAATGATCACCTTATACCGACTTCAGCCTAT |
| | | GGAAGATCTTGCGCTGTTGCGTACCTACAGCGGGTAAACGGATGTGTTAAACACGA |
| | | TAGTAATAGTAAGTTTCCGTTAGGCTGTAGTTTATAACAGTAACATAAGTGCTAAC |
| | | GAGATCAACACAATTCAAGTTGCGAAAGCAAGAAAATCTTGCTACATATATCTTAG |
| | | ATAAGTATGAAAACATAGATTGCGTTTTTACAAAAAGTACGAAAACATTATATTCT |
| | | CAAGCTCACGCTCCATGAACATGCCATGGATGCGAGAGCTACTTAATATTATCCGG |
| | | TAATTATTAAAGTAACTACCGGTTGCGCACAACGGCTTAA |
| 62 | 42.00% | GACTCTTCTTCTCAGTCCACGTTTGAAAATCAGACAACTACATATTCAATGGAAGC |
| | | GCTGAGTCGGAGTGGCTTTCCGATTGACTGCAGGTGTCTGGCGATAGATTATTAAA |
| | | ATAACCGAGGACCTCATCTGTGATTACTTATGTTAACACGTCGTTACAAGCAAAAT |
| | | GTACAGATCGTGTGTGGGTTAGGGGTTCACTAGAATCGGTGGGGCAAATTTGCCGC |
| | | AACCGATATCGTATCTGTCGCCATTTAGTGGGAGCTGGGCGTGCTATCAGAATTTA |
| | | TTTAAACGGTTTGGGGACAAAAGAGGACCTTATACTGGTAGTATACCTTCTTTAGT |
| | | CTTTGCTCCGATTGAATACACCGGAACCTAATTTGTAAAGAGGCCCAGATGTTGGA |
| | | CAGAGTGGTTATGAGTGCAGGTTTATAGTTCAAGCATCAGAATAGTATTAAGATAA |
| | | AACTGAGGGCTTTCAGGCCTTGATTTAAATGTGAGAGTATTGTCAGGCCATTTGGA |
| | | AATATCATAAAATCCTTTGTGCCAGATAGTTATGAAGCTGCTTAGATCCACTTGCC |
| | | TTCATTTGAGTCTGCTGACTGCCAATTAGAGTCCTCCTCGGTACGTATGAATAGAA |
| | | AACTTCAAATACGATTCTCCCCAATTTGCTCTGTGCAGCCTTGCCGATAGTCCTTT |
| | | ATGTCATACACTAGGTGTGAGCTCCAAGGGTCTTGGTTCCAGCCCCGCAATTCAGA |
| | | TAAACATAAGCCCCAGTAGCGGAGGAGATTTTGAATACCAAACTAACTTTATAACC |
| | | CGCGCATGGCCAGTGCCATAGCGAATGCGCGGGGAGAAGTCATTTTAGAAGCCTAT |
| | | CAGGCGATCCCGGATCATTACCCTCGTATAATAAATAGCCTTAGCTGCAAGTTCGT |
| | | GTCGCCGCCAACGTATTCGGTATCAGACTCTGATGTCCTTTAATAGTGATTATGAC |
| | | GACTGTCATAAACTTTGTAGTAGTGTATATTATCGATTGCGTTTTATTCATCTTGA |
| | | TGATGGGATACATCTGCACTTTTGAGCTAATCTAAGATCAAATATCTATTTTCACG |
| | | ATCCCGCTACTACGGCTCGAGAAAGTTACTTTACCGGACCGGGCTTAACACAAGAC |
| | | TTACGACGTCCTGGATAGAATTTTAGGGGTTTCTAAATTGATCCGGTTTGAGAACT |
| | | TCTTACTTATATTCCAGTTTCGAGGACTAGGCATTTCTTCATTAAGACCGAGGCAT |
| | | GGGTTATTTTTATATTGTGATGCAAATCGGTTTGCCCCGCCGGAGAGACTACATGC |
| | | CAGTTGGTAACGTGACAAGGCATGTGCAACGTTCTTTAGTGTCGCTACGGGATTCT |
| | | GAAGTCTACTGCTTACCTGATTATACCACGGTTCAACTTCGGTTACAAAGGATATT |
| | | CGCTATTGCACGGGATGGAAATTTATTCATGTCCCAAAAAACAAACTCGACAAAGG |
| | | TGCCCACATGCGGCCTCATTTTACAGTGCACTTATGAGCTATTGCGAGCTCCCTCC |
| | | AAATATTGGTGGGACAGTTAATAAAAACGATCTGATAAAAATAGTAGGTATCGAGA |
| | | CCTAAGATTGGAATGATCACATTCGCGTGTTATAAGATTGGAGATGTTCTAACTTG |
| | | GATGAAAATGTTAGTTACAATAACCATATCCTGGTTCGAAGAGTATTGAGATGGAC |
| | | TTTCGACATTATAATATGATTTCAGAAAGGTCGCACATGACTGATCCTTTCCTCTG |
| | | CAGGTGGTCCTGTCATCGGGTATGTTTTTTTCCTCTAGATAAATGGATATTGTAAG |
| | | CAAATAGTAATTCCTGCATGCTGGATACCATACATGATGTGACCGCCATAAGCTAA |
| | | CCAGCTTCTAAAAAAATACACTCCTTGCTAGTATGGTGATTAGTTACGGTGCATGA |
| | | AAATAGTAGGAACGCTGATTCTCGTTCATTTTGTGTGCGTTCCACGACGAATTTCT |
| | | GTTCAAAGTCCTGCAGATCTTATTGAGACCTTTACAGCAC |
| 63 | 41.20% | TCGTAGGCTAATAGAAACAGAATTATCAATTCCTTATTTAATACATCACTGGACTG |
| | | AGTCATTCTCTCAGAGCAAAAGGTAATCGCTTCATTAAGGTATTGTCTATCCTGTA |
| | | AGAACACCCACGCCGTGGATATATCTCAACATGTAATTAGGGGGTACATGCAGTGT |
| | | CGCAAAATTCAAGCGCGAACTGGGGCATTTCTAGTTATGCTAGCTAATCTACTCTT |
| | | GTAAAGGAGCTTTCGACTAAAAACTGCCACTATAATCTGATTCAATGGTGGTAATA |
| | | AGCGGTAATCTTTAACCGTGTTTTTGCTGTCCGACTTAGTGAATTGATACGTTTAT |
| | | AGGGAAAAAATAGGTCGCTCAATATACCTTAAAGATAATATCACCGGCATGCGCCT |
| | | ATGAGGTATCGATCCTGTGTCTATGAGGTAAAAAACGAGACTAAAGTTTGACTGTA |
| | | TTAATAATTATGAAAGGGAACCTTGTAGTCAAAAGATTAAGAGCAAACCCGTCTTT |
| | | CAATGACAAGACATACATTGGATGCCTCGAAATTGATTATTAAGTAACCAGAACCA |
| | | ATGATTATACTAAGAGCTTATTCCTTTCTCCGCAGACTCTTAAGAAACAAGGACAA |
| | | CTGCCCCTGAGCAACCAGCCTGCTGATACGTCCAAACAACCCGTTATCATTAGCCT |
| | | GTATTGAGCTAAAAGCACGTTTATTACTTACATGGCAAGTATTATTTATTATGTGG |
| | | CTCGTATAGGTCGGGTATAGAAATGTTGCACATTACAAGAAAGTTCAATCATAAAG |
| | | CGAATCGTTTATGTTAGCAGACTTTATCTACAGTTAACACGAGGCTAGCGAGATGT |
| | | GCTACTTTTCAAGTGTTTGGAATGCATCCGAGGTCACTATAGGCAATTCTTTACCG |
| | | CGATCAATTCGTATTTGAAACGCCCGGCTAGCCTCCCATAGATTCCCAGTCAAAGG |
| | | AATCAAGGCTGCGCCATTCTGTGATTTACTCCCTCTTTGGACAACCAACGTACTAG |
| | | CCTGCAGGATACGATGCCAACATTAATTTTTATAACCGTGAGATCAACGCGGTCAA |
| | | GGAAAAAGTTAGGCATAATATCGCGGACACCCTGGCGTGAACGATTAACATCTGCG |
| | | GGATATGAACATTTCTCGATTTACTTTAATGATACTTGGCTTCATAATAAACATAA |
| | | TACATCCCCCTGAGGTTGATAAACGTTAGAAACTTAGGCGAGTCCATAAGCGCTTT |
| | | AAAGGATCTTTTATCACACACGCGAAACATTACCATTCGATAAAACTCTTATCACT |
| | | CATCCCGAAATGCCAGTTTCGCACATGCAAAAATAAGCCTTCGAGATTGGTCACGC |
| | | CCGATCAGTCGTCTTTCGCTACCTAACCTATGATAAAATAGTTCTTAGGAGTCAGG |
| | | CAATTGACTTGCCTGTGTCTCTTTGGAGGCTTCCAAGTTCGGATTTAAGGGTATAT |
| | | GCCTGTTGTAGTCGGACAAATAGATAGGATAAGCGCTTTCCAGGCGGACTACACTA |
| | | TTAGTAACTATCAGCGAATATAAATGTACTCGGCAGCTTAAGCGTAGACTTAGTAC |
| | | TCGCAGGACCTCTTGCTCGTTCTAGCATATATCCTGGTCGTTTTTAACATTTTAAG |
| | | CTCGAAAAAGTTGTCGGAAGATGACTCCATTAGATGGACGATTAACGAACAAAGGT |
| | | CTGTGAATGACATACACATCTGATCAGTATTGGCCGCATTCGCAGGATAGTACATC |
| | | GCGGGGCAGACGTATTAAATCAACCTCTCCACACCCGGGTTTCGTTTTGCCATTGT |
| | | TGCCCTCGACAGCAGCGTTTCATTAATAGGAGGCTTTATAATACGTCCAGAAGGTG |
| | | TCAGAGGCCTACGAGCTCACGAACGTATCCTCATAAACTTATTGTGTCACCAGTCA |
| | | AGTCGTATTTTATCTCCTAAAACGACTTACCCACACCTTATGGAGGCTTAGCGATC |
| | | GTGTATATATGCTTCTTATTATAGTGCACCCTGGGTTCTA |
| 64 | 41.20% | ATTGGGCATTTCGTCGGACACTAAATGAACATTAAAGGATTGATCTTAGAGTGCTA |
| | | TATTGAATCACTCAGCCCAGTCCTTCGGACTTCCTTGTATTTCACTGGGCGTATAC |
| | | TACATTCTCAAAATAATTTTGCGAGTCAATTAAACTAGATACCACCTATGGGGGGT |
| | | TTCGTCTTGGTTTCAAATTAGATGGTAGTAAGTTTACGTGAACACCGTTGAGACGT |
| | | AGACGGCTTTTATGGGTTGTCTGTGTTAGACTCATTGAGCTGCTCATCCGAATTAT |
| | | TCATTCAGTACTATTTAGCACTTGGACATCCCTGCTAGAGCTCTGCGAAATGCGGT |
| | | ATTAGGTCTGGGGTGACCTCCAGCTCAATTAATTTACACCGGTAGTAACCAAAGGT |
| | | TAGTTAAACTCACGAAAATGATACTCACTGTTTTGTGTATCCTTAGTTATATGTCG |
| | | GCGGATTCAACCTTCGGATAATAAGTAAATGGTCTCAGATCGTAGCTGCAAAAAAT |
| | | CGTAAAGCAACTGTTGTTAAGATTGGCTACTCCTAACAAATTCCGCCTCCCTCAAG |
| | | CAGGACACTTCGGAATACAATCCGGAAATATGGCGTGAACCCTCTATGATCGACTG |
| | | ATTCCAATCACGGTTCAGTCCACTCTATCTAATTAACTTATCGGGTAGATACTAGA |
| | | AACTCACTCAAACCGTATTCGTGAAATAATTATTCGGAGTCAGTAAGCAAAGCCCA |
| | | GTGTGTATTTTACACTTAATTGGCTCTCTGTCAACTTCTTGCAAATTAATCCATTA |
| | | CTTGATAATAATATATCGCGTTCAATGGCAAGAAATCCACCGCAGAATCGCAAATG |
| | | GACTCCCTCTCATCTAGGTTAAAGCAAAAATGTTGAGATTCCACCTAAAAGTGGAT |
| | | ATAGAAGACAAAATTATTTGTACCAACAGTAAACAGGGACGGAAGGTGCCTCTCAG |
| | | GTAGTTACTGAATACCTGTTAGACGGGTTCTGCCCGGCTTCTATGACTTGAGATTA |
| | | TGTGGTTCTACAGTATATCATCCGTCTAGGAGTGAACCTAATGAAAAATACTCTAG |
| | | GTTGGTACGTATTCATTCACATAAACGGATGCGATGAGTTGGCGGGTTGGAAGTTC |
| | | TGTTAATGTCGTAAGTACTTATAGGCTGACAAGAGGTAACTGTCATACGAAAGGAT |
| | | TCGGTCTCGACGGCCGAACTCTAAAAGGTCTCCTTTTCCGGAGAACACAAGACTCT |
| | | TCTGCTTCTGACCGTATTTGGATAGATCCATCGGCGGTACCTTTGTTTGTTGGATC |
| | | GTAACATCTCTTTTGATCCTACTATGTGCCAACTCAGTTAGTTCGCGCTGAATTAA |
| | | GATTCAAGATCCTGTTCATATCTTTTATAAAACATGTGGATGTCTTAAAACTCATC |
| | | TCTTCAAACGCCATTGCTCGTTTCTGGAGTGTTACGGGTTCGGAGTAGAGTGGTAT |
| | | TGGATGTCAATATGTGAATTTATCCACTCTGACATACACAACGAGTCCGAGAATTT |
| | | TAGATCGTGCCTCCAAACAGCGCTCAAATCTTACAAATATTAATGTAGAGCCATGG |
| | | CCCCATGCAGAGATGTTACATTCGCATGGATCAATCTAAGTTTGTACAAAAGAAAG |
| | | GCACTTCTTAATCTGAACTTCATATCGTGTTTCCCTAGCGATTACTATGATTCTAG |
| | | TGTAGCGTTAGTTGCTTATGCTCTTTATACACTCGAGGTATCATGTACCAACAACC |
| | | TAGCGAAACTGATACTGAGAGGTTGCAGATAGTCTTCGACGATTTAGCTACTGTCA |
| | | TTTAACATTCCTGCCTAAAATAGCTTCCGTCCACTCACGTACTGGATCTCATTCTC |
| | | CGCGAGCCTTATAGAGACTGGATTACGTATATTCAATAATAATCTACTCTAGACCA |
| | | CCGACCTCATCCCTTGTTTATTGATAGTGGTGTCCCTAGCTGACCAGTCTTGTTGG |
| | | GAAGAAGCATGTAACATTCCTATTAGCGCCAACAACGCGT |
| 65 | 40.70% | AGAGAACGTGTCACGTACTAAGTGCAAAAGAGGCTGGGTTTTTTTTGTTAGCTTAA |
| | | AACACCAATAGACACAAATCCATGGAGATTTAAATGCAATTATTAATCTTGATCGA |
| | | ATTGTCTTTTAGCCGACAACCTGTTGGTCCCGACAATAAATTTAACGATTGTTTTT |
| | | ATCCTAAGATCAACCGTTGACGAACAAATTAGGCGAAAGTTATATTAGTAGCCAGA |
| | | CGCGTTTGGAAACAGGCAAAAACTGCTAGAATACCCGTAGAAACCTACTGGAATAA |
| | | ATGAACCGATACGTTACCGTCTCAGGAACTACTTAGGTTTGATAGACAGTGGAATG |
| | | CCATATGTCTTTTAGCGTAACAACCCTAAAACCTTATTATTGGAAATTTACCAGGT |
| | | AGGATGTCATGTAACACGCCAATCCAATTCATGTCACAAAGTGATTAGGTATACTA |
| | | GCATTTATAACTTGGGTAAGTGCATCTCATGTAAGTACCGATGGGCGTACCTCTTC |
| | | GATGTATTAACCAGCACCCACTTCATACAAGTTCATCGGTAAGTGGTTTACAAGAA |
| | | ACATCATAAATAGAAATAACACCTCTTCAGTGATAAGCGGAACCCCGTGCCACTTG |
| | | AAACAATCTCTCGCAGATGACCCTTGGAACAGGGCTGACAGTTTGAAGTGACAGGG |
| | | TGAAGTCATTCCTTTACAATTTAAGCCGGGAAATTTATCAACACTAAACGTAAAAT |
| | | AAAATTGGCGTACTGCCTGGACATTGGTCGCAATGTAATCTTCTTTGTTCTCGTAA |
| | | ACCAAACAATAATATTTTGAATCGTATTATATTGCACAGGTAAGCCACTGCAATTA |
| | | AATTAGAGCCCATCACTTCCCGGGCTAATTGAGACTAAGTCAAATTATCCTTTCAG |
| | | ACTTCTTTAACCTAAACATGAAGAGGGTTTTGGAATTGTTAAAGACATTCCATGGG |
| | | GTACTGACGTAGTACCAGCCAGAGTTCGATTCTTACAATTCACACGTATAGGTAGA |
| | | GGGTCCCACAGCTACATATCCTATCCTGAGCCGAATTCTCGCCATTGTTAGCTTTA |
| | | AATATTTCGAGCCAGACCTGTGGAATTTAGTGAGTTGAAGACTATGGGAGCCATAC |
| | | CGAAGTTGCTAATAAAATTGTTTCTAATTACTCTTCGTACATCAGAGGCACGCCAT |
| | | GTGTGTGATTAATTCATCTTGTTTCCCGTACAAGCAATAGCAATATTGCTCGCATC |
| | | ACGTCCACCAAGTAATTATTGTATAGTTACTTTGAACTATATCTCTGTAGCATTTC |
| | | GAGTGGTGCTCAGAGGCGCGGATCTTGCCTGTCGGGGATTGTGAAAGTTGGTCAGA |
| | | AAGTTACAACGGTATGGTATTTTAGAAATCGCGAACCTGATTGCGTCCTAACGCGA |
| | | TGTTATTAGTATTCAACGGTTGGTCAGAGTTATATACCCCTAGAGAGGCCTATGGA |
| | | GATAGACAGTCTCGCGTATCTCATCATAACTCTTGATCAATCTAGTCAAGTAGTTC |
| | | ACGGGACTAGCCGTACACAATAAGGAACCTAAGTGCAAAACCACTCTTTAGATAAG |
| | | GATCCTGCGCCATGCTTTGAGCCGCAGCATTCTCTCGATGAGTCCAGCGTGGTTTG |
| | | CAACACTTAGTACATAAGATAGTTAAATACAGAGCGGTCCTATTTTGAAAAAGAAA |
| | | TCCTATGGACCGCACCAGCCGGAGGTTACCTAAGACTTCGGACGAACATCCTTGTT |
| | | TAAATGTATGACTGGATGACTGATTTTCAACAGAGCGAGGTCCAAGAAAAACTACA |
| | | AGCCACTTATTAAAGACATGAGTAAGGACGAGTTATTGAAACTAAGACATACGTGG |
| | | GATAGCTAGGTGGCATAATACAAGCAGATAACCCCGTACGATTCAAACGATCTTAA |
| | | CAAGTATTTTATTACAAACGGGCCTGGTTTTAAGAGAAAAACGTGCAGTACCCTCA |
| | | ATATGAGTAATAAGGGAAGTGACAGGGAGCACTCGGCGAT |
| 66 | 40.50% | AGGGCTTGCATATCCACAAAAATGAATTTATCTAGGTTCAATTACGTGTTATCCAC |
| | | TCCAGCGAAAACTTGACACTAGGATTATTGTCTTTTGTCGACACGTTAATACAGCA |
| | | ACGTCCAAGAGATCTCTTGCTTTGGCTTGAACTTGCAATATTCACGGGTTGTTTCC |
| | | ATTCTTACCTCGACTGGCTAGCTGAATGACCTTTCACCTGGGTTACGATGTACGCG |
| | | GGGCACTGTGGCATTAAACGAAGTCATTATCTGCACCAACCCTTGATAACAAAATA |
| | | AATATGGTCTGCGACACCTTGTGCTGGGAGACAAAAATCTTCTGTAATTGGTTCTG |
| | | TACGACAGGATTAGTTCCTCTTTATTTCTTACCATGTTTCCTCTTCCAGCATTAAG |
| | | ATGGTAAATTGAATGTATAGTGCGCGATACGGAGCACGTGTCAGTTGTCGCTCGGT |
| | | CGTCGCGATTATTGCTTGGAGGATCCTAATAAAGCTAAATGAGTGGAGTAGTAGTA |
| | | TGCGTGTGTGCCGGCCGTAATATCTCATTCACGTGCATCATAGCGCATATATTCGA |
| | | CACTTGTAATCCCGTCTTTCGAAGAATCTAGGTTAAATGGATACTACTTTTTACAC |
| | | ACGCATCCTGCCTCTCGGCGGGAAATATGTTATTAGAAACTTCTGAAGTTGTCTGG |
| | | ATTAAAGTACTCATCATGGCTAAAACACTCTATTTTTGGTGTGAATATAGCTCTAT |
| | | TTACTTCTATCGAGGCCTCGTTCTAGAGGTTATTAGTGACAGTCCGTCCGTAAATT |
| | | TTCCTGTATACTCGTCTTCCTTATTAGGGTTGAGGTGTACTGCATGTCTTATGCTA |
| | | TACAATCAGCGTACGATCAAGACTGTAATATGTGTATACGACCACATTATGAATGA |
| | | GGGTAAGGTGCGATAGTCAGTAGCTGCTTGCTATTATCCTTAAATCGAATAATGCA |
| | | GCGCTTCAACAATAGATCATATGTATTTCAAGCAACAATTAGGGGATTCAACTAGA |
| | | GATGCTAATGTAGGTTTGTGAATATTTTGGTCGTACATTGGTAGGGCATCTGATTG |
| | | CATGTATACAGTCATAATTCAGAGCGACGCTCTTTTTAACCTTGGGAAAGGCCGTG |
| | | AACGAATGCGATTAGGCCAATCTAGCGCATATAGTTAATTATTTTACTCTTTATCT |
| | | CTTGAGCAACAGCGGCAAGGAAACCTGGGAGTTGCTAGACACCGAGTAGAAATCCC |
| | | TTACTTCGCCAGCGGATCGATCTGTACTACATGCATCTTCTACTAATGGTTGAAAG |
| | | TGAAGCTAGTACTTATTTGCATGGTGCACCCATTCTTACAACCAGGTTGTTCTAAT |
| | | GTCTTTTCATCAATTCTTAGCGGAGTGGGCATAATGAAAGTATAAGAATGGAAGTG |
| | | TTCTATTTTGCAACCGGAGACCACATGAAAGGATCGACACAGAGATGCAAACAGTG |
| | | CATACATTCGATGTGGCATAGACCAACTCTTGTACGATTTAATGTGATCTCTGTCA |
| | | CAATTCGTTTAGGTGTCTATGGTAAAACCTCAGCCACAACATGTATAGTCTTACAG |
| | | GCATGGCTATCGTGATTTAACCGTGAATAACTTGTCGGTAACAGAAACTCTGGCAC |
| | | AGGTGAGCGTAATCAAATCAACTTCAGTAATGAGGACTTCTAAGATAGTTCCGAAT |
| | | CTGTTCACAGTATTAGCACGGTGATTGAGTTCTCTTCTAATATTCCTATCTTTACA |
| | | TTGCGTACTGTCACAGAATGCTGTTGCCTCTATGATTTTACAACGGCAATCTAAAT |
| | | CGTCGTATCATATGTTCAGAATATTAAATAGCTCAACTCCGTGTTGAGTCCTAAGA |
| | | TAAAGATAGAAACATTGACTATAAAATCTATCCATTGTAAACCAGACTAATCATGC |
| | | AAGCACAAATTAGAGGGCAGACCGCGGCCATTGGAATCATTTATATCTTTATCGTT |
| | | TAATTCACAAGAATGGCTAAATGCCGGATTTTGACCGGGC |
| 67 | 39.10% | TTACATAACGACTCCGTCGAAGCCGTCCCGGACATCGAGTCTGACACTTACAACCC |
| | | TGAGAGCCGCTTCCCTATATGTCTATAGATTGCGAGTGTATGCCACTGTCATTGCA |
| | | GATTTAGGGTCACCCCAAAAACACGAGTATTATTAGAGACTACGAATCATTTAGCA |
| | | AACAATTTCGCGAAGCCCTAATTGAAAAGGCAACCGATTCACCCCTGGATAGATAA |
| | | GCTAAAATAGTGTTATGCGGAGCAATGTTCTCATTTGGACCCATACACTCTATTCC |
| | | TTCTGAATGACCTTCGAAATACGAATAAGAACATGGCGTTCCCAATCATCCATATA |
| | | CCCGTTCAGGCTGAGTAGCCAACATTTCGTATTCAAAGATACAGTTGACAAGCTGA |
| | | CATTCATTGATGACTTAGGGGCTAACATATCAGGCCTTTTCTTAATGTTTAAATAC |
| | | TTGCCTATTATGTGGCCATGAGGAGTGCGATGATACCAATGTTATTGGAGTATCGT |
| | | TAAAAAAATTCGGTAGTGTTATAATTACGAACTATAGCTTACGGGTCATCTATTTT |
| | | AACATAGTGAGGGCTTCTTCACACTTCCAGTCGTCGGTCTGCATGAAACAAAAATG |
| | | AGTTACATTTAGAGGAATGCGGGGTAGGCACAACTAAACACAAGGATTAAATTCGT |
| | | CGCGACAGGAGTACACTAAACGTAATTAAAAAGCTACCAGGCGAAACTTCTATTTA |
| | | CGGGCAATTACGAATCCTATGACACTTCAAGGACCTCTCATTCTAAAATAGAGACA |
| | | GCCTCCACTCGAGCTCCGATTGAGCTCTGCTCTCTTCCAAACAAGAACCTCCGTGC |
| | | GAGCAGCATATAGCGAGCATTCTTCGGAAGGACCTATATAGATCGGTCAGTTGGGA |
| | | AATCTTACAAAACGTCGAGCATATATTATTTGCCGTCCGCAACCTATGCACAGGGG |
| | | CCTTTAAATCAGTTTATTTAAAAAATCTAATTTCAAACAGTCTTGCAATAGGTTAG |
| | | GTGGGTATAGAGTATCAAAAATACGTGACTAAAAACAACAGAAGTTGATAAACAAC |
| | | AGTGATTTTCGGGATTTATGCTACACCTTAGCGAGAAACTTCTGTTAACATTGTCT |
| | | ATGCTTTGAAACTATGTAAAGGAATTCGTGATATGGTATACCTAATAGGCCCATAC |
| | | CATTAAACTGAATCATAGTGGACGAGAAGCTTTATCGCCCTCTAATGCGTAGTGAC |
| | | GAATGAAAATCAGACAACCATTATAGAAGTCCGAGTCAGCCACGGATGTTCGGAAT |
| | | TGCTATATATACGCATGACTTGCCAAAGTTGTGGTTTACTGTATATTTCGTATTCC |
| | | ACAATTACATATAGCTAAATCTACGATCGCGGCGCGGTATAAGATTTCAAACTCGG |
| | | TAAACTTGAATGATTTAAATCATCCAATTGTTTTATGGATCGTGGCCTGGAGTTTG |
| | | GCAATTAATTAAAGGATATTTAGCTGAATGTGTAAAATAATTTTTAACCCAAATGT |
| | | GTCTATAATATGTGCTCGGATAAAGCTCAGGCATAACCACAGATCTACGCGACCTT |
| | | GTGATCGTCCTTGTATGTGTATATAGAGCAACTACCAACAGTTGTTCAGACGCAAT |
| | | CAAACGATAGCTTTACGATAGGATGTTCATTTATTACCAAGTACTATTATTCACTC |
| | | TATAGGGTTATTATATCCTCTACTACTCCGGGGTGCGCAACTTTCCTTACGCCATT |
| | | ATTAACGGAATGAGCGGTAAGCGGCACCTTCTATATCATCGTCATAAGAGTGAGAT |
| | | GTAATGTTACTATGCCTTATGCTTGCCATGGTAAGCCGAAAATAAGAAGATCACAA |
| | | AATAGCACCATCTTTTCCATAGATTCTCATAAACATTGATGTTTGAGCAAAATAAC |
| | | AGCTATTACAATGATGTAAATTATTATAAATGTCTAATCATAAGCCAGTAATTTCG |
| | | TTAAGCAATCTAGAGAAGTATCTTAAGAGCGTTAAGAACC |
| 68 | 41.30% | CCTCACTGAGACCAATTATGACTTTTCTCTTGCAATTACACAATAGTGCGTTAAGT |
| | | ACTGAAAACCATCCTCAAGGCTAAATGTTATAAGATTTTTCATACGAGTGGCGAAA |
| | | ACCAAGTCAAACTGGTTAAATGATGTCTACTACAAGTTTGGGCTTGGCTGACAAAT |
| | | TTTTCTATGAGCTACTGTAATAATGCGTCTTCATACGAACGCACTCTGCCCATAAA |
| | | TAGGCGATGGACCTAATACGTCAAGCCCATCTTCAAATAGTTTTTCTTGTAAATTT |
| | | TTGTCTTGACAGACATGATACGTTAACGTTGTCTTTGACCATTATATCTTCGCGAT |
| | | AGGGTCGAGTTCGTATTTATTAAATTGATGAAATTGCGACACATATCACGTGACTT |
| | | AATCCCGAAAAATTAGAGTTCTTGCGCTTGTCATAGGCATGAAAAGCTCCCCTCAT |
| | | AATACGTTTGACCTTTAACGTATGTCTTTAACATATGTTCCTGGTAACCAGGATTT |
| | | AAAGTCATGGTCAGCCTTCGAAAAATGTGAGAAGATCGCGAATACATCACGAACTC |
| | | TCTCAGGCAAACATCTCATCCACCATTTATATAGTAGATGCGCTACCCACTGTTAA |
| | | CCTGTTTGAGATGTCGATTTAAACGTTAGAAGGTGGTTCCATCGCTGGATTGCAAC |
| | | CTTTACTTAAGGTCGATGATACGTACAATCGCTTTACTTTAAGCTAAGTTATTGGC |
| | | ATACTACTGAAATTCACTTCCTGGCAGACTTGCGTTGCTCTCGCAATCCCGCAGTC |
| | | CTTTATGATGTCTAGGCGTTTTACAAATCGACAGTCATTGTATTAAAGTCATTGGA |
| | | TTGTACGGTGTAAGTCGACAGGGAACGTGTTGAGTTAATAGTAAAAGGTTCAGATT |
| | | CTTGCAAGCGCGCTTTTCTATCGCCTGGTTTATCAAACTCATGGTGATTATATATT |
| | | TTGCAATTCATCAGCCCTCATATGTTGGTAAGACTCGGATTGGGTCGACGCCAGAC |
| | | TAACGTCATAAATGTTAGAATTATTAAAGACGCAATTGTTTATGATACTCACTAAT |
| | | GGGTCGTTAGATACTTATTGTTTTAAGGCACCAGCCTCCATTTGTCCGAGTCCAGG |
| | | CCCGAGCTTGGGCGCAAAACTTTTAGTATCTAACTGTGAGTGACAACCTTTAGAGT |
| | | TCTCTCGTATAGAAGGTCCGACGTCAGAGTATCATAACCTACTGGAATTGGCCGGG |
| | | TTCGCGTGCACTCTCACTTCCTGCCAGAACGCAATTAAGCATGCTGGTAGTCTCGA |
| | | CCCGGTACCTCACTCTATCAAATGAAACTATAGTATACCTATCGATCTTAAGATGT |
| | | GGGTTCTAGCTGTGACTGCCCGAAGAAATAGTATTTCAACGACCCGATCGTCTAGG |
| | | AGCGTTGTGGGAGGGTTCAATGCTCTCGTATCGATTCCCAAGACGTTGTGGACATA |
| | | CTAGCTGGCGAATAATACTATGTGTAGTGAAGTTTGCGGTAATCTGCGTAGTGGCT |
| | | AATTAAGAAACACCGAGCCGTGTCTTTTGCAAACTCATCGAGGCGTTGACTAAAAT |
| | | GTCTAACGGTTAGGGCGATATTTTATTTTTACCCGCGGTTTATTATCTATGAGTAC |
| | | TCCCCATTCCCATATAGCGTGCATAGTTTACTTTTCCATATGTTATTAGCAGGCTG |
| | | TCCGCCCAAACGTTGCGCTAGCCACCGTTAGATCACAGTCATATTATCATAACGAT |
| | | TACCAGGTTATAGTTTCACTGACTAAGGAGCCCATAAATGTTCATTTTCACTAGAC |
| | | ATGCTATGGGTTTGGCCCGACCAAGATTGATAAACTGCGGTAATGGCGATATGATT |
| | | AAACGATTAAACTTTTAACTACCATGGGGAGACAAGACTTCTTAACTAGTCGGTAT |
| | | GGATTGCTGCTTGTAAAGCTAAACAAGCTGAATGTAAGAACAGGCTGGCCGGTTCA |
| | | TAACACTATCACGAGTGGCTGACAGAGTTTTACTTATAGT |
| 69 | 40.30% | ATTCGCATTGTTTGAGTAGCCGAGCACTAGTGGGATCATTTACCTTCTCGCGGAAG |
| | | AGTTACAAAAGTACTGAGGAAATATGTGAATTGTTATAGCTTTTAGGAAAGTAAAC |
| | | ATGAAACAAGGTAGAACAGATGACGACGTGATACAATTATTTACACAACTGGAAAA |
| | | TTCCGTCAAAGTTTTAAAGTATATTCCTTGAGTCCTATTATTGAATATTCGAAAGG |
| | | TAGTCACCTGAGTTGTCCCGTAATAATTACATAAGTATCCGTATGGCAACAAATAT |
| | | CTCCTAGATCCGGGCCGCGGATAGTTTTCGCTAAAGTATCTAAATCGAACTTCTTA |
| | | GCATACGATTACTAGACTATCACCTTGAGTAGTCTATATCTCTGCGAGTGTAAAAT |
| | | GCACACGCCGTTAAATCGCCTAAATGCCTTTCCGTGGCCATTATATGCCCCACTTG |
| | | CTTTCAATTCATTCCATAAACTATGATCATGGACCCGGTTGCGAGATGTTACAGAT |
| | | AAAGTCGAAACTTTCAAGAGCAGCTGACGACAGGTAAAATTACGATGCACTGCGGT |
| | | GTAAGGAAATAATCTCCAGGTTGCAATAGACATTTAAATTGTAGAGGAATAGAGTT |
| | | ACGCAAACCAAGCCCAAGGATCTACCGAACCCCTCTACCTTATACAAACTCGTCAG |
| | | CCGAAATATACCAAATAGCACGTTGCCTAGAGGTTTACATTAATCATTTTACACGA |
| | | TCCCTTTACTATTAATATATCGATTCCGATCTAAAAGGCGTTTCAAGGATAGCAAT |
| | | AGTCCTATCAAAATCATTCAGTTACTGGCAATCCAACCAATTCGCTGTACACGACG |
| | | GGGTGAGGTCGTAAAATATTATATGTCATAGATGCACTGTTTGCGACCATGTCTAG |
| | | CATTTTTCAATAGCTCCACCCACGCGTTGGCGACCCATTGTTATTCAAAAATGGGC |
| | | CGCATGAAGAGTTAATTCGTCTTGTTCTGACATAAGTGTTGACCATCAGACAATAG |
| | | ACGTATACCGCTGGTTACCTCTAATCGAAGATCCAGAGCTCCTTATGCAACGTATA |
| | | GTAAACCTGGCTCGGAAAGGGGTTACTCTTATTTTTAGCACCTACATTCGGGATCA |
| | | AATCATATGCACTTTCAAGATGGTGCTCACTATAACACAATAACTTGGGTTTCCAG |
| | | TTAGGATGAGGAATCCGCCAGGTTACTCTATGAAGTCAAGCTCTTCCGTAGTTTAG |
| | | GCGACGCTTGACCCGCGTTCCTCACAAGTAACGCGACAGATTGGAGCAATAGCGAC |
| | | TGCTTCACCATATAGGGACTTACATACAGATCGAATGATTTGCAGCTTTAACAACC |
| | | CATAACGATCTGCACTAGATGCGATGAGATCTCTGTAAAACGAAACTTGGAATTAC |
| | | CCAGAGCAGTTCTAATTAAGCTTTTTCGATAATATTACACAGCAACTAAATGAGCA |
| | | CGTATGCTCAAGTGTCGCAAAATCCTTATTGTATAGGAATAGGTCGTTGTCACAAC |
| | | ATAGGTCTGTCACCAAACTCAGACATTATAGTACTTTACGGAGCATGTTTAGACAT |
| | | AATCTGCACAATGCTGATTAGTCTCAGTGTGGTCAAATTCTTTAACGTCTCTGTTC |
| | | CAATCAAAGTGAGCAGACTGATTGCATCACAACTCCATCACTTAACCAATTATTAA |
| | | TAGTCCACACAATTCATTCACTCTTCACTGTTCAGCACTCAGTCATGCTCTGGATA |
| | | TTCCATATTTCCCCGCCACATATACTGAGTTTGGTCACTCATATGTTCGCTAAAAT |
| | | CGATTTTTAAGCCATTCTTGCCTATTAACGACGGTCCTAATCGTTTCCCTTCACCA |
| | | TGGATATACGGTACGGGCCCTATTATCTGCGTTACGCAATGTCAATAAAAGATATT |
| | | CTAAGAAGAAAAAAAGATAAGTTGCGTAAGCGTGCTGCAAGAGACACTCTCTCTTC |
| | | GCAGTAAACTAATTTTTCCTTTAAGAATACAAAGCGAACA |
| 70 | 39.10% | GGATTAGATTGTGCCATAACGCAACAGGTAAAATTATTAGACCAGCAAAAGAATCC |
| | | TAACGTATACAATTTTATCGTACATAACCCGTGAATCTTATTAAACCCAGCCAGGC |
| | | CGCCTTACTTTGCTCCAAGTAGGAGCATAATGCATAGAAGTTTCAGTATCCTGTCT |
| | | AAAGCTATTAAGTCGAAATGAGACAAAAGTGACGAGTTATTAACGATCAGAAACTA |
| | | GTCTAAAGGGAACCCTCCTGCGGCCATTTCTTGAGGACTTACGTGCACCATATCAT |
| | | GAGGTCCTACTGTGGGAAAGGAAATCCTCAGTTTACATGATTTGAAATACTGTAGT |
| | | GACCTGTCAATTTACTGATTTCTATGCATAAAATGACAATCTCACCGAGTACGCAT |
| | | AAATCAGCGCAGATCTCATATATTCATAATAATCTCCGGGACGTTATTAAATTAAT |
| | | TTTTTTCTAGACAGATATTCAGAAGTCCGACGTTATACAAGTGCCCAGTAACATGT |
| | | TCTGAGCAAATAGATTGTCGACAGCCCCAATTAACCACCTACTAGTCTTTAGGCAC |
| | | TGTGTGAATGAAGCTATTAAGTACTAGACATAATGTCATTGCTGGCTCTAGCTGAA |
| | | GAGTATACCTAGCTTTTTTTCCAGATTTTTGAGTACGGGATCTGTTCTTGTTGAAC |
| | | AAATAATCTGGATGGCGCCATACAGGCGTCGCCTGGAGCGTCAAGCTCACATACCC |
| | | TATCGTCAAAGTATGTTCCGTCAAAGGTGTCTCAGCACTTAAATACTTAAACAATC |
| | | CGAGTTTCGAGTTCTAAATGGTTGCACAATATGCCTGGTAGATTGATATAATCTTG |
| | | AAGCAACGATGGATGAACAAAAATTATTGATACTTACTTTTACCCACACAAACCGT |
| | | CTGAGTGTCTTTTTAAGAGGGTTACGAATATATAAAAGCGGATCACGATATTCCAC |
| | | CGGGAATAGCGCAATTAGTCATATGGAACATGGTGTGAAACCACAACTATGAAATC |
| | | TATCCGTACACCAACCAAGAGACCTAAAAGTTTTACATAATCCGTTTGCTTTCGTA |
| | | TTGCCCTCTATCTAATGAAAACCCATTGACAATTATAAAGAACAAAGGTTATCACA |
| | | CGCTGCGTATTTAGAGAAGAGAGGACATGTGGGATCAATGTGGTCGCAAAAATTAT |
| | | CACTTTAATCAACACCGATTCTAAGAAGAAATAAACGTCGTATTCAAGGGTACTGT |
| | | ATAGGTACGTTAAGCGTTGTCGTACACTCAGCGATTTAACTAACAGCCGGGAGAAT |
| | | GCATAATTATGATAAAGTGAATCCACTTAGCGTCTCGAATAGAGGCTATTTCGCTT |
| | | GCAATCAAATGCTTAAGAGTATCCTAACCAATTTTAGACAAATATCAGTATGTTTA |
| | | TCGATTAAGCTGGACAATTCCTCTACACAGATGTTTAAGCGAACTAGCATTTTCAT |
| | | CCTCCCGACTCATAGGAGTCCTTCGTTGCACAGTAGATAGTCAGCGTGTGTTCTCT |
| | | TCTCCAATTGATATGCTGAAAAACTATAGGTTACCCGTTTCGGTCGGATAAAGAAT |
| | | TTGACTTAATTTTCTTGCCGATAGTAGGTATACTGTAAGGCAGCCAATATAACCGT |
| | | TAGAGCTTGATTAGTATGATATTCGCTCCTTTTAATGTATCTACATCTAGCTCTGG |
| | | AAAACCCGGTGTAGAAGTAATGTATTAAGTCTGCGAAGCGGGAATCTGCTTGTGAC |
| | | AAAGATTCTGTCGCCCGCAAACGTCAAGTAATAAATCGCAGATACGGTCAGAAATT |
| | | CCTTCTGCATTTCAAGATTAGTAATCTATTCGATTCCAAACATCCTGCTCCTAACA |
| | | GAATGCGCACGGGACCTAATGAACTTTTCATATACGTTTCATCAAGCAGTAGTGTT |
| | | CGGAAACGAGACATAACAGGGTACATGTGCATCAACCTTTAAAAACCAATCTCTAT |
| | | TTGGTATAGTCGTATTCGAAATCCAGTAGTGAGGTGAAAA |
| 71 | 38.70% | AATTGGAGCCAACCATAAATTGGATGGTAGTTCCAAAATTTTATAACCTATTCTAG |
| | | TGTCTGCAAGTATTTAGGAGATAGGTGAATTACACGTCGTACACATAAATATGATA |
| | | ATGCGATCAAGAGTGAATGGGGTCTATAGTAATATGATGTAAAACTTAAGGATATT |
| | | GTGGACTGATTTAACGTTACGTAGTCCTGACAAGAGTTTAGATGCCAGGTCGTAGA |
| | | AGTTGTGTATCCCCCTATTCTCCCAATGGTAGATACCGTGATAAAAGATAAATTCC |
| | | TGTTAAGGAAGTCGAGGATGTTCTGTGGAGTGCAGAGTTCTACATGTGATGAGATA |
| | | ACCTAAGAGAAAAAGTAATTTATAGATTGCCCCCGTTAGGAGCTACACCCGACTAT |
| | | TTGTTTCGTTAAGATATTTGTTCGTACCATGCTGTTATAACGACACTCCCTCGAAT |
| | | CTTATTTTATGGCAATTAAAGATGTTACAGGTGGCGTTGGCAATTCTGGTAAACTC |
| | | CGCACTTTACAAATTGTTGTTTGCAACTCTCTCATATTGTATGCAATCGACCCCAA |
| | | ACCCTCATCCTCGACCCTATGAATGAAGGTTTTCTGTGCCAAAAGCCATTTTACTC |
| | | AAAAATTAGCTTTTAATTTGGGGAGCTTAATAGCGAATTCCAGAATCGTTTCATGG |
| | | GGATTAGGAGATATATTATAGGAGTCCACCAATAGTCTATTGACTTAGTGGTTTTG |
| | | GCTCATGCACGGTGGACAAAACTTCAGGCGTGTTATCTAATTACAACCCGTATTCA |
| | | TACATATCAGGGGTGTTGATTTCAGAGAATAGATTAGGAAACTACGAGCAATACCA |
| | | ATTTTGAAGATATGGTCTACTAGTAGCTCACTTACTCAACATTGCTACTTTATTCG |
| | | AAGGCCCATATTGAGGAATACTGTCTTGTTGAGTAAAACGATACCCGTAACTTTAA |
| | | ACTATAAAGGCATACCAGAAAAAGTGTCACCGCAGGAAAATATAAGAACGTCCATC |
| | | AATATATGATGCAAACTAGAGAAAGAGCTTGATAAATTATCAAACTAGCACTTCTG |
| | | GGAATACTCCGTGGTTGCAAGGTTACAGGGTTCAGTCAAAGAGTTATTAAATCGAT |
| | | TGATATACTTATTCAAGTGATTGATTCTATATAGCTACGCATATCTGCTGACTTTT |
| | | TCGAAACGTTGCCTGGTTGTCCAGAGCATGTTTTGGACGAGAAATTTCGCGCAGAT |
| | | ATCATGATTACGATTGGCAACTAAGGATGACTAGCGTAATGAGAACCTGGCTAATT |
| | | TTGTGTTTCTTATTCAAATTGTATAACTAGGTAAGGAACGACTCGTTCAGAATGAG |
| | | TTCTAATCATAATCTTCTAAAATACTGACAGAAATAATAATATATATTATGACTAT |
| | | TCAGAAAACCTATAAAAAGCACTCCGTAGAAGCTCTTCAATCTTAGAATCCTCACC |
| | | TAGGAACCTGAAGATTATTGTATTGACTTATTTTGTAGTTATTAAAGAAATCCAAC |
| | | GACGGGGACGACTGCTTGTATGTAATATTTCCGTTCCACAAGCCGGGAGTAATAAT |
| | | AAGCAACCGTAGAGGAGCAATGGGTTTTTATCTCACGCACAGGATGTCGGAGTAGC |
| | | GAGCCGTCTGAGTATGTTATCACCAAAGATATATGTAATATGGTTAATCAGCTGAT |
| | | TTAAAGAGAACTTCATCCCAACCTCGACCGACGATCCGATTACTGTTTATCGTCAT |
| | | ACCTTACGAGATGTCAGGTCCTCGCACAAACCGCCACAAATTCCTTGTCACTGCAA |
| | | GAATAAGTTTGTCCGCAAACTGTCTACGCGCTAGGTCGTTGTATGTATTGATGAGC |
| | | CCTATCCTTATGACACTCGGACTGCTAGCCTTCTGAGATTTACGACAGGCAGTCTA |
| | | GTATTAAACCCTTACTACTTTTTGCTGTATATTGCATTGCAAGTTCCAACAAGTTA |
| | | ATGAAACACAAACCGTGATCGCCTCACCCCACAAAAGGCT |
| 72 | 38.80% | GTAAGGGTCGAACCTCTGATCATATTCGATTACTAATAACTCCAGATATATAGAAT |
| | | TGAGAAAGGCAAATGTATTTTAAACAGCAAGAAACTGTTTCAATTCGGCTTATCTG |
| | | ATGTACATTTAATAAATAGAATGAAGATCGAGTATTAGAACTGATATGAAAGTTCG |
| | | TAACATCAGGACGATTAGAGTTTATGCATGCTAACAGGAACTGACCTGCTGACATT |
| | | ATATCATACAATTTCCTGCGTCCCGCTTATGGATGGCGTCAATAGGCTAGTAACCT |
| | | AATTGCAGCTTAGAATAAGGAGAACCAAGTAACGACAACAAAATGAAAAGCAATAG |
| | | ATGGCGGACTGCGCTTTAATTGCATTGAAATACTCTGGGCTTCAAGTGTTAGTTCA |
| | | TTAAAGCTGTCTCGCGATACACAAACGCTGCGAAGTGGTTCCGGAGTAAATGTGAC |
| | | CAATGTTAGACAGTGGGCCCGCCATGAATGTGAAGTTAGTTACTAGGAAGAGTATT |
| | | CTCAGTTTGGTGTTTACTAGAGGTGTGCTTGGCGTTTATCTGGGATAATAATTGTA |
| | | ACTCAATTCTATTCTTTTTCGTTTTTTCTGCTCATATCGAAGTTTTGCTCGCCTCA |
| | | ATCAACGTTGTTTGTATAGCACTTAGGATCACTCTGCGCATAGGGAATGCTTAAAT |
| | | CAGGGAGTTCATCGGTGTCCATCCTGCAGGGACATGAAAGCTGTCATACACGGACT |
| | | CGTACCGGTCTGACAATCCGCTTTGCCTCATAGCAACTATTGAGCCGCATTCGCGT |
| | | GGAGCTGAACTATCAGAATGGCTAGAAAGGATAAACCTGTGGTGGGTCCACGAGAT |
| | | TGGTCTTCTTATGTTAATATTAGCTCACAAAGTCCAGAGTTAGTATCCATCTCTTC |
| | | CAGTCACATGGAATTTTACTAATTATTGTGGTATCATTATTATAAAAATGACATTA |
| | | TCTAGCATGACTCCCTACCACTAGTGCAGAGCTACTATGTACATAACTCGCTGTTT |
| | | ATGCGATACTCCAACAAGTAGATACGGTAATTTCGATATAGGATGAAAAAACCTTC |
| | | ATAACAGCTTAAGTTTAACTTCGAGGGTCCGTGTAATCGGACAACGCACATACGAA |
| | | GTGGCACGACCTTTCATTTGGGCTCCCTTTTGCAGGCTAGTAAACCTAGTATACAT |
| | | GAAAGCCGTCTTGCTTGTGCCTACGGCTTATTTCGTTGAACGTACGTCTAATAGTG |
| | | CCAAGGAACGAACACACGGCTAGATCATAATATTACTCCAGGTGATGGTTTCGGTA |
| | | TTTGCAAAGTAAAGATAAGTTATCTGATTCACAACAATCGAGAATTTGTCCTGTTT |
| | | GAACGCCGAAATATTATCTTACTATTGCTTTACTCAGATACCTCCAATAAATTATA |
| | | AAATGGCTTGTTTGAATGTGTATCGAAACCGAAAGCTATATCTTTTGACCGAATTA |
| | | ACCAAATGCTACGCGTTTGCTGTTTATTATGTCCATCATCGCTTTAGGTTAAGCTT |
| | | AATAGGTTAGGGAAAACTACCAGCATTCACATAATATCCTATCTAGGAAGTTAAAT |
| | | TCACCCATGTATACTATACTACTTAGTCTACAATATTTCTGCTTTATTCTTTATTT |
| | | CCATTATCAAAGTATTTCGGCTCTTAAATGGGGCAATTACGAAAGATATGATTCTA |
| | | GCTCATGCTCAATTGAGATGAATTTATGACTTTAATGGGGTGTACCATTTAATAAT |
| | | GCAGCGCTAACATAACGTGCGACGCTAATATCATTTACTAATAGATTTTCATTCAC |
| | | TATAATAATTAATAATCTTCTGGCCCCATGGCACAGGCAATTTTAAATCCGTACCC |
| | | GTCAGCCCTAAAATGCCAAGATTAGTGAATCTGGTGTCATACAGGACTAACAGGTG |
| | | CAAAAACCGGTTGCGTCATCAAAACGCAGGATTTACTCAGGATCTTAAGAAATCTA |
| | | AATTTTCGCAGAATCGCTCATCGCCAAAATTTTAGGCGTC |
| 73 | 42.80% | CACGTGGTTTTCAGCGGTTAACGCAATCTGCATTATTGGTAGAATTTTACACTTAA |
| | | CAAAATATCACCACGCGGACAACTGATTTAGCAAATGCCGTCCGTGACGCGGGACC |
| | | CGCAGCACATTATTAGACATAGTACATCAGCCTGTAACCGATCAGTCATCACATAT |
| | | CCCGGAAAGATTTCAATCCAGTTGTAATCAACGCGTAAAGTTATATAATCACTTCA |
| | | ATCACCTTACTAACTTCAGAATGGCAGCCTAAAAATCTGATGCTACGAACCGCATG |
| | | GTGTTGAATAAATTCAATAGAATGGAGCTCCTGGATATTTCACGACGCCGGGACAG |
| | | AAATAGTGTTATAGAGAAGAAGGCATGCCGTTTTACTCGATTCGTAAGTAGTTTGA |
| | | CGAAGCAAAAACTTGGGGAAGAACTTATGAGTTAGCCACGACAACTACCGGGAGGA |
| | | TTTGCTTTTCTTCCTCCATGCCAATCTTGGAGGGAGTACCTCAATCACACGATGAA |
| | | TCAGCCTTAATGGGCGCCCAAAACATTCTTGGTGCCAGAAAAGCGGATGCTTCCTC |
| | | GAATGTGTAATCAGAAAAGTGGTAGATGAATCTCCGGCTCCATCATGGATAGAGCT |
| | | GCAGGTATTGGTGCAGCAGGAACGAAGGTTCTACCAGTAAGTAAAGTTTGACGTTA |
| | | GTTACGAGTCTAGAAGGCCCAAAGGGCAACCAAAACGTCGGCACCATAACATCTAC |
| | | AGGTGGTAGGCTAATGTAAAAGTGGTTATAATTGCTAGGCAGAAATAAGGCCGTTC |
| | | ATTGGGCATGTGTACACTCCATTGATGGAGCTTAATTCCTCTCAAAATAATTACAT |
| | | TCTGTTAACAAGAAATAACTTATTGGTCGATCTACGAGCTAGCAATAAATAATCAT |
| | | GACCAAAGAGCTGTGCTGTGATCAGAAGTTATGACGCTTATACAGAGAGCATTGTA |
| | | AAGGGCAGGCCGAAGCAAATTCACAGAGTACCTGAAGCGAACAAAGGAAGAGACTT |
| | | CTTTATAATTTACATCGCTTGGCAATTAAAGAAGCGAAACACAGTTGCTCGAATCA |
| | | CATCCTTACGTGTCGTCGACAATATCATAAGCATTACTAGTTTAGAGAGGTGAGAT |
| | | ATCGGTAGTAGGTATTAGAACATTCTAATACCTAAAGCTCATTACTATTAGCACCT |
| | | TTCCTCACCTTATTTGGATTTCCCGCACGCCGTTCGCACCGAGCTAAGTGCAATAA |
| | | GCCATGGCGATGACTTAGATGTCACATTGCCCCATGAATTCACCCCAGTGAGTTGA |
| | | GACGATTTGAAGTTTAATACGTCGTTCGTGGACAGCTTGAATGTTTCACACGTGGT |
| | | AAGTTGCATATGAACATATAGGAGGGGCCACAAAGCTTATGCGTGAAGCAAATATG |
| | | ATTCCTCCCTCGATCCGTTAATTAGAGTTGCTGAAGGGCATAAACTTTAGCGAGTT |
| | | TGTATTAACATAGTCATATGAAGTAACAGAGACCCGTCATAACGCTTGAAAACCTG |
| | | AACTCAGAATGCGCTTTGTGTACCATAGGCATATACCCCACATTACGGAGATGATA |
| | | ATCGACAAATGCTCCAAGAAGTAGACCTCTAGCCATCATCACGTGTCTCTACTGTA |
| | | TTCTCCGAAGTTCCGGAGGCCAGTTCTTAAGTAGGCACAGAACACACGATGGATTT |
| | | CCTAGGGACGTACGTATGTTCGACTTCTCGTCAGTAATCGCGACAGAAATGGGAAG |
| | | GTGAGCTTAACCTAACCCACATTTTTGTCATGGGACTCTGTGAATGGTGTTTCTTA |
| | | TGAAGCTATCACGGTGTAAAGATATCTAGACACGCTATGTGCTACTCCGATAACCC |
| | | TACGTTTAGGTTTACGAGATTGGAGAAATATACTTTATTAATTCTTCCCTGGAATC |
| | | GTACCAACAAGTTCCAAAATGGCTCTGCGGTCTGTCAAAATATGAAGGGCTCAACT |
| | | TGACAGGACGACTGACCGGAAATGATTTAAGTGAACCTCC |
| 74 | 38.00% | ATAATTATCGACATAGATGTGCTTCACTCGATTTGACAGCTGGATAGTAAGAATTA |
| | | GTGTATAACCCAATACGTATGCTAATACAAACCCTGGACTGATTTGAATGTAATCC |
| | | TATTCATAATATTTTAGCTACCGTAAATGTATTCTGCAATTGAATTTCGTGTGAAT |
| | | GTAAAAGGTTTAGAAGTTTCCTAAGTTATCGGGTGACGTTTTTAATGGGTCTTACC |
| | | GTAGATTCAGACAATCTTTTGGAAACCAACTGAAGAAGGAAATCACACGACCTGGC |
| | | GGATAAGGGTTTGTAATTCGCGTTAAAAAACTGACGTTTGCTATAAGAGACGTTAA |
| | | TGTAAATGTAACGCTTTAAATTCTCTGTGCGAGAGTTTTTTAAATGAGATCAAGGA |
| | | TTGTTAATTTCAGGAAGCTCCGTTATTGGATTTTGCCTTCTCATTCGTCACTATCC |
| | | CTCTCCGATCAATCCGATTGAGTCCTAGTGTAGAAAGTTCACATAGAAAGCAGTTT |
| | | TCCGATTAGTCTAGCGGGGTACTAAGTGAACACTAGTCAGTTGGTGATATACTATA |
| | | GCTAGGCTGTGATAATGTTAATCGGTTTGTGCCTACTGGAATGCTTAATTTCATCT |
| | | TGAGGACTTGCGCTAGGAATCGGTATGTCTTCGTTAAGTCCAAAGTGCCTTTTCGA |
| | | CAGATGTTGGATTGATGCACTCCTCCGAAAAGGAATCAAATTGGGTTTATAAATTT |
| | | TGTCTTTGTGACACCTGCCGAATTTAGATCTCACCATTATCCACAATAACCCTATT |
| | | ATCTTTACCTACTTCCGTCGGAGCTTGATTATGAATATTGGCAGAATTATGTAATA |
| | | GTCATTAATATGTTGAATAAAGATATCAATACATTCAGACAATTGAATTAATCCTG |
| | | CGTAAAAACCTACTTAGGACGAGTTGCTGGTATTTGTTTTTATAATGGTAGACATG |
| | | AGGGACATATTACGAACCTCTGTAAGCCTGTTCTGATGTGGCCGGCGATCACGTTA |
| | | CCTGATGAGATTTATAGATCTCAAGTCGGATGTCCTCTTTAATAAACTGAAAAATT |
| | | GACGACTAAGTGGGCTAATTATGCCATCAGAAATAAGCTAACCAAACCTCTAAAGT |
| | | CGACCCTGTAGTATAACTGGCAGTGCTAGATATCACAGGGTGTTTGTCTACTGAAA |
| | | TTTCGGCATTCTGGTCACACTTATTGCCGATAGGTTCTAGTAGCTAGTTTATCTAG |
| | | ACTCCAATTGAAAGCTTACTTCGGCCTATCAGGTTGAATGATAGACGGTCTGTCTT |
| | | AAGAAACTACAGGACATATACTGCATCGAATGCGTTTAAATCCTAACGCAGAAGGG |
| | | TTGTTATCTGATCATCAGTAAGCACCAATCTGCATGATTACAGACGTACCAACAAC |
| | | TGAATACATCCTGCCTCCTGAGAACTAGAACCTATTGTATTGCGGATGAGGGTAAG |
| | | ATAGGTAGAAACCTGCTGCCAACTTATCGATAATAATTATGAACCATGCGTGGGTG |
| | | TTGATATAGACTTAATATGACCTCCTGTCTGGTTCATATACCAGTTTTCAATGCTT |
| | | AAGAGAACTAGCTTGTACGGAGTTTTTTTAATACAAGTGCTAAATTAACAATTGTT |
| | | CAAAAACAGTTTATAGTAGTAAGGTATTGTACCAATCGTATAGCAATAAATCATAC |
| | | CTGTGTTTACTCCATACTTTCTTGATTATCGGGCACGAGAAGAGGACAACTCCCAA |
| | | ACATCAATGTAGCCATAGTGAATGAAAAAAGTCGGTTATGAATCGTTAGCTAAATC |
| | | GTTTGCTCCAATTAACAAAACTATAACCTAAACTGGTGAACACATAGATAAATGCC |
| | | AACTCGTTATCGTGTTATGCTATAGATCCGAATTTGGTGGTTCTCCGAGTCTGTAT |
| | | CGTTTTTAATCGAGATCTTACCTTATTCCTAACCACATTTCGTAAGCCTATTGAAA |
| | | CGGGTATTGCCGGTTCGCCCATCTGGTAGTACGTAAACGA |
| 75 | 38.20% | GAGGTTAGTGATCAAGCGCATTAGCTTTTTACTGCGGAACGCATACAGGATATTTA |
| | | CGCTTAAAAAGGTGGATTTCGTATTTATTAAGTATTCTCTTTACTGAATTATTGTC |
| | | CATCAGTAATCGCTGGCTTTATGAACTATCAACATTCGGTGTTGTGTTAAGTTATT |
| | | AATGACACATGCTCGACGTTCCCCAATTCCCGTGCGTGATATATTATCATATGACC |
| | | ATTAAATGATTAAAGGGGCATAATATTTTGAAATAACACTATTAATTTGAAACTTT |
| | | TGTCCTTTTCGCACTACATGTTGGTAACATCGCACGCACTAAATACTGACATATCG |
| | | TGCACCATGCTTTCTAATAGCACTCCGTTCCAGTCCATAGCTGAGACTGTCTTTTC |
| | | GGACAACACAATAGATAAGAGTCTATCTCTCATCAAAACTGTAAGAAAAGCTCTAC |
| | | CATAATTGGGGCCGAAACGTAATACGATTATTATGATATCGCTCCTGCCGAGGTCA |
| | | AACACCATAGCACTCAAAAATGGTATCCAATTTAGAGGGGCTATGAGTAGTTAAAA |
| | | AATAGGAATTAAGGTGGCAACAGGACAGAAGTCAATAGGTTCCCTTGAAGGCTAGA |
| | | TTAACAGAACTGTAATGTGACTGCCTGTAAGCGCACTGGAGACATCAAGTATTGTA |
| | | CGAGTATAATTGCACTTTGGAGGTACAACATCGCACTCGACTCTTTCATCGATATT |
| | | TTTTCGTGGGTGAACTTGAGTTAAAGTTGATGGTCCCATTCACAACGAGCGGTTTT |
| | | CGCGATGTAAACGCCGGCCAAAGACAACCTAACGCCGAATTATTCTACTTCATATG |
| | | CCTAAGTAAGCCCGTTCTTTGGAGAAGTCTCATCCTCTATTATTATACATAGTTAT |
| | | CATATTAGTCTAGTCGCCAAAGTGTGGTTTCTAATTGATAAATATAATAAGTTAAA |
| | | AAATGAGAGCTCAAAGTTTTTCCTTACCGTGCCGCACAAGTAAGTAGTCTCAAAAG |
| | | GACCGCGTAGGGAGGGAAAATTTAATGAGTTCTAATATAATATGCAGGCTTGTGAA |
| | | AGCTGACATTGACTACTCTGGACTGGTCGGATAGTTGCTAGACATACCTATTGTGA |
| | | CAAACTGACCCATTATCGAGTCTAGTAGAACCGGTCCGTACAATTACACATTCTTC |
| | | GTAAACTAGTTCTATAAAGACTAAAAAAATCTATATCACTTGGAGAATTATGGAAG |
| | | ATGAGTCAACTCCGAAGTGTGGTCAAAAATATTACAGATTGTATCAAATCGAATAG |
| | | GCCGTAAACAAGGGGTATACGTTCACAGTACAAAATAAATCAAAGCCTTCAATTAT |
| | | ATCGAGAGATTATTACACTACCGCTGCTCTTGACTAGTCAAACGTACCTCTCATTG |
| | | ACAACATTCAGCATGATTATTGCTCCATGTCAAAGACTCCGTGTTCCCATTAGTTT |
| | | TAAAGGCATAATTTATCTCTTTTCCTCTTGGATAACGAGAGATAATTAGACAATGC |
| | | TAGTTTCACCAAGCCCGACTCGATAAGTGGCGGTTTTAGCCTACCCAATCGCCTAA |
| | | ATATATCAAAAATGACTTGTACGCGATAATACTGCTCGGGTAGTTAACGGCCAAGT |
| | | ACACGCTCACAGAACAACGGTTGTACCGCTTATCTAATTAGGGAATGTACGGCTCT |
| | | CTCACTAATATGCGATTAATCTATTTTGATTTTTATGCAGAGCATCCTAAGTGAAA |
| | | CTCTAGATGCCGCCAATTTTTGTTTATCATTTCGCCAACCGTGAATTCCAAGATGG |
| | | CCCGCCAAAGGGCGTATAAATCGAGTATTTACGAAGTAATAAGTTAATTCTAAAAT |
| | | TCTTTAAATATGAAGACAAACAATGAATTGATTATGATTTCCAGATATTTACTTTG |
| | | GTACCGGATTAAACCCATTTGAACGTCATTCGATATCAAAGTCCGCTAATAAGGGT |
| | | TTCAATTACAATTCTTCAGGAGAACACATCGGTAACCTTC |
| 76 | 38.30% | GCGCAAACCAGCAAATTAGGTTTGACCTTCAACAACTGTAACTCGATCTGCAGACG |
| | | AGTGAGTAACAACAGCTACTGGTACAATTTTTTTGTACCGCAGCATTCAGGTATTA |
| | | CCCCTTCACGCTCAGTACAGAGGTATCGGGCATCCGTATAAAAAATTGACTTCTTT |
| | | TTACGATAGTCCAATAGACCGTTAGCTTCTACTTCATAGTACTAATAATAACCTAA |
| | | TGCAATAGTCTGGATAACATTCACGGGACACTGATACTAGAATCAACTACGCTGAT |
| | | GAGCATGTCCAGACTGACAATCGGTCGACATGAGAAGGAATAGAAAAAATCCTACC |
| | | CTGTTAATTCTGGTCATGTTTGCTGGTCTCTTTCCTACTCGGTGCTTCTCAAATGC |
| | | CACATATTCGAGCATAATACCTAGTTATAGGCATAAACTTATTGTTGCTGCCCATG |
| | | TTGAGCATTTTTTATATTTAGGCCTTTTACGAATTTCTGTTTCTATTACTAAAGAT |
| | | GTCAGAGTAATACCACCTTCAGACAGAATCACATGATTAAAACTATAGAATCGGCG |
| | | GTACAAAGATGTATCTCACCTATAGAGTATGCTGATAAAATCATAGACCCTAGACA |
| | | TACTATTCTTATCGCCCCTTAGAAATTATTGTAGGGGTTGCGATTACAACGCATAC |
| | | GGTATTTGCTATATGAGCACTCATGGCTTATGTGTACAATTTATTGATATATATAT |
| | | TTAGAGCTCCGGATCGGGTTACAGAATCACTTCACGACCCAGCAAATGCTAATGAT |
| | | TTAAGCGTAGTATATTGGCTTTGTGTCCAGTTTTCACTACGGGTTCCTTTCTATGT |
| | | CCTGATAATCTGTACAACCGACATACCCTGAATTCATGCCGCATATGTCGTGTTAA |
| | | CAGTGATCTAGGGTCCAGTGATAGGGTCATTTTCGTATCGTCGCATCTGTATCGAT |
| | | TGGAAAAGAATTATACAGTCCGATTATCACTTAGAACTACACGAGGGGACCTCTTA |
| | | TCTGCCCTACCTATTGGAGTTAAAGTTCTAACTGCTCAATCTCAAGACGGCCGAAG |
| | | ATGGTTTTAAAATGACGGTCCACACATTTACAGACAAATTGGAATGCTTAGATATA |
| | | TCCTACTGTTGATTTTTGTCCAAAATTAGAGGCGATGTAACCCCACTGAAAGATTG |
| | | AGCAGTACAGTAATTCTAACTTGAAAAAATAAATTTTTGGGTATGCTCAATCTTTA |
| | | AGGTGACCTACTAACAATATCCTAGATCCCATACGGTAGTTCGACAGAGATCCAAT |
| | | ACATTCTAATCGAACATTAGTAAGTTAAATAATATAGAGCTACATTTCTAAGTAAA |
| | | TCGATGCTTGAAGATATTGGTAGTTCGCAGAATTTGCATCCATCACAAACACTAGT |
| | | CTTTACGTTTGCCAATTGCTAGGTAGAGTAGATTACGAGTCAATCAGAAGACCAAA |
| | | TTTTTTGACCCATAGGATACAACACGTAGTCATGACAATCGCATATCGCTAGTATG |
| | | TTAGATCTAAGAAAATAGTCTACTTAACCGGGTCATACATCTCAGCTATTAACGAT |
| | | ATTATGTTGCCTTATGTTAGACACGTCAATAAGTAGAGCATGCATTTCTGCCTCAA |
| | | ATAACAAATTTGTTAATATGCAATGAATACCTGAGTTGAATGAACCCAAACTAAAC |
| | | TCAGGGTCCTTCCATAGCGAGAGCGCTAGGCTAACATGAGATTCTGACGTCTTCGT |
| | | GAGTTGACAGGATCTTGCCAACAAATTACATATTTGAATAGGCATGTACGATCCAT |
| | | TATACTATGAGTGCCAGAGAAAACTCTGCTGGCCGACCGTTTTACGGGGGGAAAGT |
| | | CAAATATGTAGTAAGTACGAATTTTCCTGGGAGACTATAGTTGCTGAACGTTCTTA |
| | | TTCTCATTTTCTTGAAGTTAAGGATGGTAAAACATACTATACCTATGTAGATATTC |
| | | TTTGGTAGTATAACTATTATAGTAGCGTAGACGTTATGTG |
| 77 | 39.50% | GCCTAAAGACCTCTATATTTTAAGCTAGCATAAAGGCAGGAGACGTTCTAACATCG |
| | | CACCGAGTTCGACTATGAAGAGAGGTATTATCAACCCTGTCTCCCAGTTCACACCG |
| | | GTTGCATTATCATGACGTTTTTGATTTGTTTTTTTTGAGTAACGGGTTCATTGTAC |
| | | GTTCGATAGAGTACTCGATAAACGACTCATTCCACGCAAGCCTATTTTGTAACTTA |
| | | TAACTAGACATTAGTCTATGGCTACTTTCACACCCGAACTTACGAACAACGAGTAT |
| | | TTTTTTTTTGGCAAAAACGTAACGTTCGTATGTGGCCTAAGTCATTAAAAGACAAA |
| | | TATTGAAGAAAAACCCATGATTTAATACCGATAGGACATTACAAGGGTCATTAGAG |
| | | ATAACAAATAAATTAGGCTTCTTCCAAGAGTTATCCGACTAGTTGTGCTCCAGATC |
| | | TGCGATACTGATCGAATTTATACCTCATTAGACATTCGTAGTCATTGGTGTTGGAC |
| | | TTGAAGTTCTGTACAATCCTCGGTGATCACTCTTGGACAACCTGCTGATAAAACAT |
| | | GTCTATCGTCAGTCCAGTTTGTATAATAAACTAATGAGACAATATACAAAACAATC |
| | | CGTGGCACTACATGTTGTATACCAACATAAATTCTGAAGACCTATGATTCTTGTGG |
| | | CCGAATAGTCAACAGATTTTACGATCACTAATAACCATATATCTGTTACTTGTCTT |
| | | CTCAGATAGGAGCGGACTAGAAATACTCACTTATGTTATTCTTACGTTACTGTGCC |
| | | AGACGAGAGGTTTTTGCAGACTCTATGGTTTGCCGGATCTTGCTAGGAAAAGGGTA |
| | | ACTGGTGCCTGATTGCATGAACTATGTGGTATGACTATAGATGAAGCATCCGTCAC |
| | | TGAGCTCTTCGAAGTCTTTTATGAGACAAGAATATTCTTTGATAGAATCATCTATG |
| | | TCTCAATTTAATCAAGGGAACGGTTGGGTACTAAATCGAGTTATCATGAGGTCCTA |
| | | TCGGAATGCATTGTATTTGAGCAATATCTATAACTGTAGGTACTATGGCGGATATT |
| | | TATTTTCCTTGCTGCGACTTCATGTAGCAAGTCGGCAATTCCCCGCGGTTTTACAT |
| | | TTTCTGCTTCGAGGTATTAAGGCCCTAAAGTTGTATATATTATAAATTAAAGATCT |
| | | GGATTATTAACTCAGTGCAGAGGGCGTAATCTGACGTGGCGACATGTAGATGAAGC |
| | | TTGCCCAAAAGATATGAGATCTTAATATCTATAAGAAGTATGCCTACTGTTAATTT |
| | | TGGGGAGAAATGCTACCCCGGACAATTATGCGATTGTCAAGCGAATATCTTGATTT |
| | | TATCCTTGGAATAGGTATATTACTTCGGTTACACCAGATATGAACCTATCTATTAC |
| | | TTCATATTTTACTCAGGCTTGGTCGGGACCTGTGTTACTTTAAAGGCATTAAAACA |
| | | TACAGCGTCGACAATCCTCCTAATCAATATCCTCAGAAGGAATTTACTCGCAATAG |
| | | CGAACTGAGTTTTTTGCCTGTACAACGGTCGTGCCTACTCAATCATTGCCGCATAC |
| | | TAATCTCTATCATATTGCCTTTACGGGGCGACCAAGGAGGAATCCTATCTAATCCC |
| | | AGGGCACCTGGAACACCTGCGGAACATGCTTCAATAATAACATCGTATAAGTCTAT |
| | | GTCTGCGCTTGTGACGTCATAGTACTTCTTCTAGTGATATATTACGCCGTTGGATT |
| | | GGGATCACGTTTAGAACGACACTGTGAACTTCTATATGTACTCTTTTCTCACGATA |
| | | TGCCGTCGAGTTTTTTATCGATAATAGGCAGTGTTGGAGCGGGACGTGTCATTAGT |
| | | AATAAGTTTTTCCTATCAATTTCCTGCGATACTTGACTCCTTTGGGGCAAACATAG |
| | | ACGACGGTTGGAGTCAAGGTGAACCAAAATAGAAGTACCTGGGTAAATGCTTCATA |
| | | GGCACTTGGACAAGACATTAAGTCGACACACTATGCCTTT |
| 78 | 38.10% | AATGTTCGGTCCCGGGTAAGCTATCATTCTATAAAAGTCCCACCCCGCTTATTTAA |
| | | GATTCACAGCGCCGCAATGACGCGGAACAGGGTTGTCTATGATGACCTAACTACGG |
| | | CACTTTAGGTATCATATATTGAGTTGAGCGAATGGATCTGCTAGGCTTCCCGTCTA |
| | | TCGGATGCTTTAATGCAGGTTAATGGCCCGATTGAAGTTTATAGTATATATATACA |
| | | CTGTGATGGTGTAACTACGTTACTTCGTTACTGATCAATTTTCAAATTATCTCATT |
| | | TGTTAGGCTACAACTAGGACTAAAGCTCAAGTAACCGATGCGAAGAGGCCGAGATG |
| | | GTATAATCAACGGGGGTGTAATCTAATATACGAATCATGCTAGGAGAGCAGCTTAT |
| | | CGTCAAAACTCTGTTGGCCAGATTCTAATTACTCTTTATTGTATCTTTTTTCATGT |
| | | AGATTAACCGTGAAGACAGTAGTTCATGTACGTTAGTCAATTATTGAGAACATTAG |
| | | CTTGAATGGACGCGTGCTCAAATAATACCCCAGTAATCTAAACCATATTGTTAATC |
| | | TTTTACAAGACCCACCAATGACCTAATGAGTTCACCTCCACATACCTGTCATTAGG |
| | | TGACCTTATTTCCACATTTGTATTAAATACTAATAACTGACCATATTGTGCTGTGG |
| | | TTCTGTACACTTGTATACCTGTTCGGCTAATACTAGTCAGTGATTTCATAGCGAAT |
| | | ATAACATTTGACAAGACTGTAGCAACAAGTTTTTGGTATAGGGTTTGTTAAAGCAT |
| | | ACCGCGCAGGACGACCGTCTCTTACATTAATTTACTCGTTTTAATCTATAATTATC |
| | | CATATAATCAACTAGTCCTGAGCCAAATCTTCAATTTCCCCCGCGTTTGAGATTGC |
| | | TTGATGAGGCGAAATAAGAGGCGAACGGAACTCCAAAAAAGAGCGATCTTTTATCA |
| | | CGTCCCTCCATAACGCTTTATAAGTCATTAGTCGGCATCGTTACAAATTAATGATA |
| | | GACCAGAAAGTACACAGACGTGTCTTTTATCCTGTAACGACCCTAATTCGGCACCG |
| | | TCTACTAAATGCTTTGCCGTACGCTCTGATGATTCTATCCAGCGATTACGTATATG |
| | | TTCCGGGGTAACTACCTAAATCTAATGCGGCCATAGGCCCATACTGATCCGCCGAT |
| | | TTCGCGCACTGCTTTACTTATATACATCAGTACTACTCGGGCAACCGGTAAATAAT |
| | | TTACAATAGAAGTTTAAGTGCAGTTACATGCTTAAGATATCGAGAGAACTTGTGAA |
| | | ATACGTACACTAGGATTTTCTCAAATTCGTGACATTACAAGGTCTGGTTTCGCGAT |
| | | TCTCTTGGACTGATATAATATGATTGAAAAATGTAGTAGATATGATCCTGGATAAC |
| | | ATTTTTAAACAAGTCTTGGGTGAGCTCGGTACCTTAAATCCGATCATAGAATACAA |
| | | CATGGCACCTACATTCATATTAAATAGTCTATTACATGATAAGACTCCTTCATGTC |
| | | TGAAACATTGGTTAGACAATTCGCGGTTTCAGTGGGTAGCGTGTTCTATTGACTTC |
| | | GAAATGAGAAAGTGTTTCGGCGCGTACGGTATATCTTCCCCCATGATTATACATAA |
| | | CATCCTTCTAAAAATCGCGCCACTGCAGGGTCCTCTTTTCTTATATATTATTGAGG |
| | | ATTTGGACCGATCAAACTTAATATTAAATATGATTCTACATACAAAGGTAATGATG |
| | | GCAATCTACTTGCGGGCTCGACTCGTAGTCTGTTCAATGAAAAATACATTTCTCAA |
| | | GAAATAATCTTCGAGCTATTTCACTCTGTAGTTAAAGTTTCAATCTTGTTACATAC |
| | | TGCTTATACAAATTTAATTTAAAAGCATGTGTCAATTTAAGGCTAAATGCTCAGTG |
| | | TAAATTGTATTGGTAAACTCCCTAAGACTAATGAATAACTTGATAATGTGGATAGA |
| | | TTAAATCCGTGCAAGCCTATCCTAAAATCAATTTGAAGTG |
| 79 | 41.00% | TACAAATTGTCCACGGGCGTGAAAACAAGCCCATTCTTCTTCAATTGCAAGATTTG |
| | | CGATACTTAAACCTTACTGATTTAATAATCGATTCAAAACGCAAGAGTCATGAACA |
| | | GAACGAGACCCCGCCATATTTAAATGCACATTCGTGCAGCGATGGGTATATTGAGG |
| | | CTGTGAGAGGCTCAATTAAACATTTTACCAGGAGATGGGCAAAATAATGCGTGGGG |
| | | ATCGCGGGACTATAATCTAATCAGTCATACTCTAAAGTGAGCTTCGTGATATCTTG |
| | | AGGATAAAAAAGGGCCTAAGCGCACAGGGTTATTGAGTTCCAGCTAATGATGCTCG |
| | | ATAATAATCGGCCGTAACTTCAATGCGAAGAGAATATACGATTCTGAACAGTTACA |
| | | GATAAGGCCTATTAGGCGCGAAAATAGTCGTCTAAAAGAGGAGAACTGCTGGTCGA |
| | | GAATGAGTGGGGGTTATTCTAACAAAGGTAGCTAGGTGTGGTTATAAACGAGAAGG |
| | | ACTACACCCAATTGATCTCGATAATAGGGCGGGATTGTTTATTGACAGTAGTGAGG |
| | | TGTTCTAATAACAGAAATTTAGTTAAGGTGCGTATTCTTGGAGTAGAGCACAAAAC |
| | | CCGCTAATGAGCATTGTATGAATCCGCGACAAAAGAGCAAAGATCACAGCAACGAA |
| | | AGTCTAATTGAAATAGTCCTCGATTATGCCGGTGAGTTGAAAAAAGTTGTACGTTC |
| | | GTTTATGCCGTTCTAGATAATTTACACATCACATTCCTCACGTAACTACATGATTT |
| | | ACCTACTATCACTTCCAATCACCAACTCGGATTTAGGAATACTGTAACTTATTTCC |
| | | GATTATCCGATTGAGACCTAAGCAGAAAAACATAAGATGCCCATCCGAATTGTGAT |
| | | GTGGATACCAGTTGTGATAATTCGTCGGATTGAACTCAGCCTGCTTACCGCTTTTG |
| | | ATCGCAGTCGCCGCGGGTAGATGTAGTTAGCCTCACCGGCTGGATACATATCTCCA |
| | | GGAAATCGCGGAGTATCAATCTCTAGAGTAAATCCCCTGCCTTCCGTTGATCGTCT |
| | | TGCTCACCTAAATGTCTGAACTAGGCTGAGAACACAACCATACTCCGGCCACGTAG |
| | | ACGATGCTGAATATTACGCAGCTATACTCAAAGTTAAACTCTTCTCAGTGATTTAT |
| | | GATGTAGCTTAGTGATCTTTACAGATTTGGTATCGATTGGGAATCCAGTTTAAAAC |
| | | TGAAACGACATATAGAAATATGTACCAATCTACCAGCGCAAACCGAGTCGAAGTCA |
| | | TATTATACGGTAAATCACCATCGTGTGATATATTGCAATTTGAACTGATTTTTAAT |
| | | CCCTAGCTTAAATACTTCATTGATTTCTCGCCTTTAATTCTCTGAACGTTACAATT |
| | | TTTCTGCCCAACGGTCCTCCTCTAGAATACCTCGAGAGCCGACACAAATACAGTTA |
| | | GAGAATTTTTGGTGATTTGTGCGACTTATTAGAACCACGGGGTCATGACCTTAGCC |
| | | CGAATAGGTAGTATCCGGATATCTGAAACTCCAGGCAGTAATAATACATTGCCGGA |
| | | ACGACAATCGGATCTAGTGAATGCGACATAGACGGTAATATGTTAAGCACCTCATA |
| | | GATGATTACTATCAGGAAATATCAATTTAAAGCTGCGATGAAAGGGTCAGGACCCA |
| | | GCCCTTTCAAGTCTACGTAACTCCACTAGCCACATTGTCTAAGGGTGCCAATCATA |
| | | GATCATGCATCAACACCGGCGATACGCTTGTTCAGGCATTCATATCTTATAGTTAT |
| | | AAAATTTGTTTATCGTGTGCAGGGGTCGATTTTTCTCACTTTCGGCAACCAGGAAA |
| | | AGTAGTAATTACTATATAAAATGAAGGCGAATTTCGGATTACTCTGCAAAAAATCA |
| | | TTAGAATACACATCTAGGATCCGGAGGTATCTGCCTCCATGAAGTTAACTCCATTG |
| | | TGGATATGATGCGAGTAACATATTTAGGTCCGAAGAAAGG |
| 80 | 39.30% | ATCATCTACCTAAGACAGAGCTGACCGTATCCATTGTCAATAGAACAGCAACGATT |
| | | TTTTCCATCGCTGGAAGAGTGATGCGCACTAGTTCATTTCGGACAAGTAACTTGGA |
| | | CGCGATACAAGATACAATCGATGTCACAGCCTCTTTAGTACATACCATGGAATTAT |
| | | GAATCGACTAAAAACGCAGACGTATAATTCAGCTGATCGAATGATTTCGATTATAT |
| | | ACCGAAGTCAGTGACGAGAACCTTCACTTTGCGGGATACCGAACTCTGTCACAAGA |
| | | AATAAGTATAGGTTAGAATCCAGAGAAAACATTGAATATTATGTTTTTTCGCACCA |
| | | AAATAATCCAACGATGTTACGCTTAGTTAGTGGATATCATGACTTCACTAAACACT |
| | | TGGATTGTTATCTAAAGTTTTTATCTTCCTGGCTGCGACATTGTTTATTTAAGACG |
| | | TAGTTAAAAAAGTCGACCACGGAGGAGGAATTACATCGTCGCTGATGAGCCCATTT |
| | | TCGCTAAATGCAGTCGACTACGAAGAGTTTTTCGCGTATCGTCAACATAAGTTGAT |
| | | CTTTTTAGATAACAAACAAAACTCTTCGCATCGACGTAAAACATTTTTCATAGGCG |
| | | CTTTTTACACCGAAGAATCTCAGCTTCAGAATTGTACGATGTCTTGTCACAGATAT |
| | | CCTTTAAACAAATAACTAATAGCGTTGATTGTTTGACATCTACTCCTTATTGTTAT |
| | | GAATGTATACCATATTGTTATATGCTATTAAATCCCACATATTGCGGTTCGCACTA |
| | | AAATGAACATCTATATAACTTGACTGTTACTTGAATTAGTTATGGTCCAGCTAATT |
| | | TTTCATTCTAGGCATTTAATCCTTTATGTTCCATAGTTTCCTTCGACGCCTTGAAC |
| | | GATGGGTGCGAGTCCGACGGACTAACATTTATAAACACATTTGTGGGTTTGGGTTT |
| | | GCTACAGATATCTGGACGCAGGATGTTTAGAGTAACATCTGTTGTCATTTGGCTAG |
| | | CAAAATTTGAGTTACCTGATAGACCTTCCTCATTCCCTTAATATTAAACTGTCTTT |
| | | CTCGAATACCGTTCGCACAGGGTCCAGGAAATGTGATGTTATGACGGCGTGCAATG |
| | | GTTAGTCCTTATGCAGGAGTTTCTCCGCACCCATCAATGCCATTATTTTACAGTCA |
| | | AAAAAACATAAACTTGTATGACGAATGCAGACCTTTGAACTTTTGTTAACCTACTT |
| | | TTGTAAAACCAGCGAACCCTAACAGTTATGTAACGAGATCCGTTAACCAAAAGCGG |
| | | TTATCCGAGGATAAGCTTCCTACGACGTCACATTTGTCATCTTCCTTACCGGTATG |
| | | AATTGTATGCAGGTCCCTATTCGAAATGTGGTTATAACTGATGGGTATCAGCAGGT |
| | | TATTTATAACGCGTACTTTATCCTTGTAGGTTAGTTGCTCAGTACGCCCAAATCAA |
| | | AGAGGAGGCCGAGGTGCAGGAAGGACCTGACTGACAATCGTAACTAAATTATCCAA |
| | | CAGGATTGTTAATTGACAATGTTTACACTGACTATGGCAAAAATTGTCTCCCAAAC |
| | | GGCTGCGGACAGCGTTCTTTTTATCGATCTGAGGTAGCACTTGCATATGGATATAG |
| | | CAATAAGAAATAGGGAGATACCAGCGAAGAACGGAGTAGATGCCTGTGACGTGTGC |
| | | CGACCTGACATTGATTATCGAGCATGCGGATTAAAATTCAACAACTATTCCCGTGA |
| | | AGAGTGCCAGCCTGTAGTCAATTATTGTGGATATTATCTAAGTTCAGATCATACCT |
| | | CTCGTCGGTGAAAACAGATAGAGGCCAAAGGGCAAATCTATTGAATGATTGACAAT |
| | | TTGATCATATACGTGTCTAAGAATTAATTGTAACGGATGCGAATTCGTTAATCTTC |
| | | CTGGGGTACTCTTCTCCACGTCACGAGAGATAACAACAACATCAGGCTTCTGATAA |
| | | ATAGCGTAACAACGTATTATCAAATGCATCCTGTCTGTAT |
| 81 | 38.10% | TTAATGACCCCTGCCTTACTGCATAAATCTCCTAATTGTGTAATCACTCCTCACTC |
| | | AGATAACGCTTTACGTATGGATTACCAAGTAAGTGAAATCACTATACAAGAGATTG |
| | | CCTAATTTTGCTAAGTTAGCGTTGTTCGTGTTTTATAATTTTATTGTGAGTCTTTC |
| | | ACCGAAGTAGAAGGAAGTAAACTCGCAGTTTCTTATAACCACTTCTAGGCGATGTA |
| | | GACGACATAGAAAATGGGGTAAGGAACTCATAATTTTTAAGTCAATGATACAGCCT |
| | | TAAAAGATAAAAATTAGATTACCGTTTAATGAGGGTACGTGACCATTAACAGTAAG |
| | | AAAGCCTGCAAGCATGGGACAGGTGCTATTGCAGAGCTCATAAACGAAATGTCGCT |
| | | TGGGCGTCCTGCACCAGATACTTAGTGGCGGATGTCAATAGCGAGGACGAATCATT |
| | | GGATGAATATTAGCTAGTGGATACGGAAAAACGTGACTACGATTGCGGCATCGAGT |
| | | TCTTAACCCTCTCATGGAGGCATCTCTCGACCTTACACAGTGAGAGTGCATTTTGT |
| | | TCGCCAGTCTACTATGACACATTAAGGCTCAAACACGCTCTGCTTATTCATTTGGC |
| | | CTTGGGGTTCTAGATCACACTACAATTGCCCTTTGCAAGAAAAACAAATGTCATTG |
| | | AAAAATTAACTGCTGTCTTATAAACCTAAACTACCAGATACTGTAATTGGTTTTAG |
| | | GTTTGAGCATCCACCAACACCAATAGCCAAGATTGTTAAACTCTAATAACTGTCTA |
| | | ATACACGTGCATATTCATAGTGAATCAGTGCGGTTCATTTTCTGAAGAGCTCCAAT |
| | | CTGAACGATACAAGGCGTCCTGCGCGTGGATTAAAAACAACTTAAGCGTTACGCAG |
| | | AGCAGTATTCCATTTTATAATATACCGTTTGCCGCAGGAGGTTATATTGTAGAAGA |
| | | TTAGTTCATTTTGTGGGGGATTTACAGGCCAATATTTACCAAATTTTACGAGGTAG |
| | | TTGAACCTAGTGTTACTTCGTGAGGCTCGAACGGTCTTCCCGCTCCAACTGTACCT |
| | | TTAGATGGGGGCTTCTTTGGATGTAACGAAGTACCGGCTTAATATGAGACGTTTGT |
| | | ACGCGAGGCATTCTTATTTAACCCATACTTAATCAATTCAAAATTTATCTTGGTGA |
| | | GTAGCACTGGAGAATTTGGTATCCATAGCGGACCGATAGAAAGATTGTTATACCAA |
| | | AATTCATGAATGACGCTTAGTATTTTCTAGTTTGATAACATGGTTAAGACTACATT |
| | | CTATCCGAATTCTTATTAAAATTGAAATGACGCATTGCATGCTGTGATTCCAAAAC |
| | | CATGCCGACAGGAGGTCTTCTTAAAAATTCAGCGTGAGGTTACTACACCTTCAAAA |
| | | GTGCATAATTGGTGGACAACTAAAGGATAATTGGGTAAGATCTTTCTACATTCCAT |
| | | TAAAAAATTCTAACAAACCCTATCTCATGTTAAGTACTTATGTTGCCTCTTACTAC |
| | | ATTGACCCTACACTCAGATATGATAAATTGATGTTTAACCTAACTATTTAAAAGCT |
| | | CAATACCTTCCTTTTTACGCGCAATAAAAGGTTAGGCACTTTTAATGTGAAATTTC |
| | | AGCGAAATATTCGATCTTGATATAACTAAGTTTACAGTTCCTATTACTACTCATTA |
| | | TAATAGAATGTATGGGCTATGAATAATAAATGGACCCTTAGAAGGATAAATGCATT |
| | | GATTCGATGCTAGAGTAAACTGATGGCTCAGACAGAATCATGCCCATGGGGAAACA |
| | | TAACACCTAATCAGCATCAACTAAAAGTCACATGTACGAGAGCAGAATCAAATACA |
| | | AATCAATTATATAACGTGAACGTAGAATCCGGACCAGGGACGTTTCTACTCTGACT |
| | | ATATTACCGCCAGCTGCTATAGTAATCGCGTATGGAGCATGTATTTGCTGACTAAT |
| | | GCTAAAGTACAACATTACTGTGTAATTTAAAATGCTACCT |
| 82 | 40.40% | TGTACTTGTCTTCTTGTTTGTCACATACGGACCCTAAATGACCTTGTCTAGTTATC |
| | | CGATACACCTTGCTTAAGTAGCCTCCCCTAGGGGGAACTTATTACGGAATAACAGT |
| | | TTTACAGTATTAATCAAACTCTTATCCACGTTTTCCTGTGATCACAACGTATTGTT |
| | | TCCCTTGATTTGTTGAGAATCTCTATTGAGCCTTTTATCTATTAGAGTCTCCGTCG |
| | | CACATAATCCCGGTGCGTTGAACAGATACTGGCTAGACTCCTTACTTTTCTATCAG |
| | | TTGAACGGAGGATACGAGCTTCAAAATAATGATTTGTTTGTAGATGTCAGAGCATC |
| | | GTCGTGAGAGGAACCCGGATAGGGGGAATAACAGGTAGCGTTGCGGTTGCCTGACT |
| | | AAAACCCAGGACTCAAGTTTCATTATTAACATTATTTGCATGAATGACAGTGTCGC |
| | | AGATCTGGTATAATGACCAACGATCGTTTAGTAGATAAATTCCAATCTAACAAACA |
| | | CTAACCAGTATCTCAGCCCACATTGCATCTTGTTTTAGCAATCCTGCAGATATCAG |
| | | AACCCTCCTGCAGTGAATTGACTAGTGCACGACGGTAACATATCTCTTTAATAGCG |
| | | CACCGTCCTCAACGTAGATGTTACGTCTGGGGTTATATTGGGCCGGAATGTCCTGG |
| | | GCTTGGACTAATGAAGGCAAAGGCTATAAATGTGCTTATTATTTACTTCTGCGTAC |
| | | TTATTTGGAGAATGTCATATTAAAGATGTCGCGGTGGTCGGATTAATTGAATAATG |
| | | TGCGACTTGGATGCACCTCAATCTTCATTGTTTTGAAAAGTCTGGAGACGTGCAAT |
| | | TACACTCTATATGTCTTTGTATTAATCGTTATAAGCTCTAAAGGAGATAGCAAGCT |
| | | CGGGCAAATGGTAGATTAATGCTTCAAGAAAATACAAGCCTGGGGATTCACATTCC |
| | | GAATATACAACTAATGACGCTCTCATTCTCTTGCAAGTATAGTAATCGGCCCGCTA |
| | | CTCTATGGGGAGTATGGCATCAGGAGAGAGTATCATTGACATTCGAAGTTTGCATA |
| | | CTGAGCAATAAGCGGGTAATGCTTCAAAACAAAGTGCACTCACTTAATGTCGGACA |
| | | TTGTTTATAAGTGTTAGCGCTCAATTTTCCGCAATCACGCTCGAGCACTAATAGTT |
| | | GGAGTTCGCTTTAGTTTGATAATAACAAATATGACTTTGTCGCGAGATTGCCTATT |
| | | TGCATCCAGGACTATCGAACGCAACAAACTCGTGAAGAGGCCGCATTTTAACTGCA |
| | | GGATAGTAAGATCTAATTATGAAATACATAGTCCAGAAAATCATTCGAGACTACTT |
| | | AACAAATAGTTTCAGAGGTTCTAGACTTTCTCAAATGTATGTAGTTCGTGAATATG |
| | | TAGTTATACTCAATTACGACTTTGATTTTTATTTACCGCCTAAGAAACTTGATTGA |
| | | AATAATCTAGAAGCCTCAATCCTGCTCCATCACAAACATAATATACTGAAAGCTAG |
| | | AGGGCGTTACCACAGTGGTACGTCTAGATTCCAAAGCGTGCTAGGAGATTAGTGGT |
| | | CGAAACGCAGGTTCCGCGAGCAGTATCACCCTACAAAGTAGCTGGTTACAGTCAAC |
| | | ACCTAGCAGCAATTTCTTCACTTTTGTTACGATACGTCCGTGGCATGATCGTCGTT |
| | | GCCTAATTCTACGACTTAAAGATACCGAAAAAAGCAAAATCTAGAACCATGATAGA |
| | | GCTACAAAATCCCTCTACCCGTTCGTACGTGCTTCCTAATCAGATCAACTATGTGA |
| | | GCGACATAGTTTTAGCTAGTACTTGAGCGGGAGTTTTGTTCTCGTCTCTGAATATA |
| | | TAAAGTGTTTAATGAAGTGCTATGAGGGCCACTCATCTTTAGCATACTAAATCATC |
| | | AGACATAAAGGTCACCCGAAATAATCAAGCAGAAGACTAACAGAACATGCTAAGAG |
| | | AGGTCTTTCAACTACGCACTTGATAGATAACCGTTAGCTC |
| 83 | 40.40% | TCACGACGAGTGAGGTCTGAGACCGTCATCAAAGATCGTAACACTTTTTACCGGGC |
| | | TGCCATAACGTAAGATGCATGACTGCAAGAAAGTTCACGGTGGTAATTTCAATGAG |
| | | TCATTGTCATTCCCTGAAGGACGTATAATACTATGTTACGTAGATTATTAGGGATC |
| | | CTTATGCGTTGAGGAGATATCTTGCCTTGAGTGAAAGAAACTCATCTGTTTAGAAA |
| | | CATACCAAATATGTCAGACACGGTCGGCTTTGATAAGAGTCCCTAACTAATTGGCT |
| | | GCACATTACGATTCGCCGAAAATATATGTTGGGAGTAGTGTACACGATTTTAGACA |
| | | AATTCCCGAGATGATGACCGTGACATGTACAATCGCACTAAAAATCCCCGGTATTA |
| | | GACTTTGAAGTGGTTTTGGTATGTGATCTTAAGCATATTCACTATACTAGCATAAC |
| | | AATGGTGGTTGCTTTTGGACGCAAGTTCTGAGTATATGACTATGAAGCGGAATCGA |
| | | TTAATTATGTCTTCCAATAAAGCTTAGAAGTATGGTTCGTGAACAGCTTCCAGTAT |
| | | AATTTAGAGAGGCCGACAATATATATAGGGTTTTATTTACTATTGGCCAAGAACAT |
| | | CCTCAGTCGATCTAAACTTCTTCCAAAGCACTAATTCTATCGCAAAATGGTATTAT |
| | | AACAACACTAATCTTGGAGTCAACTCATATACGCGCGTGTAGAGTCATGTAATACT |
| | | CAGCGGCTAACTACATGTATTATGTCAAGTCTTCCTTGCTATGAATACTGGTATTC |
| | | CTTTGTGGATTAAAACGGTACCGTCATGTAATTTTGAGATAAAGATCTAGGACGGG |
| | | GAAGAAAATAGTAATACGGTATGTATGCGTTGAGTTGGGTCTGGATATTCAGTCAA |
| | | CTATGGGTAACTGAGGACTTTGACGCTGCATCCCCTGCTGGTGCGTAGTCCTAAAA |
| | | AAAATTCTCTGGGACAATATGTCTTCACAAGATCCTTGTGAGAATCCCGCTTCCGG |
| | | TCCGGCTGGGCCATATAGACTCCTATTACTTTCAAACTTCGCACAGAATCTTAAAT |
| | | ATGAGATTGTAAGGAAACTATCAGATCTGCTCTAGACACCGACGGAGGAGCTCCCG |
| | | GAACGTTCCAAAGCTTTTTTTTCTAAGTGTTGCACTTGGCCGGTCGTACACGCAGA |
| | | GCGGTAGATAACCCAAATACAGTTCTTCTCTATGTCTACGCCCATTATGGGACGCG |
| | | TGGAGTCTCTGTGACGTTGACGGTTTATAGGTTAAGTATGCTTACGGATGAATATT |
| | | AATGAATCGTCGTAGTTATTGAAGACGGCCGATGTAGTATGCACCGTCAGCCGATT |
| | | CCAAACTAGTATCTTGCTCCTGAGTTACTCTGTTAGATTCCTGTCAGTTTATCCAT |
| | | TTTAGTGTAGAAATATCCTTGAATGGTTGTACCATGGCTCCTAGAACTAGACAAGA |
| | | TAAAATGTTATACCGTCTGGTGAACATTTAACCTCGTACTTATCCGGACTAATGGT |
| | | AATTGTCGACCGCCTCCTGAAAACTCGCATTGGTGTCGAAAAAAGCAATGAGCGCG |
| | | TATTTTTATGGAGATAGGTGCATGTATTAGTCTGTATTCTTAGATGCTCTGTCGAT |
| | | AACATGATGTAATGCGAATTGATTAGAACAATCTGAGAGGCTGAAATTGATTGCCT |
| | | GCCCAAACACGATACGGTTCGATAGCTAGCTGCCGATGCGCTTCGATATTAAACGT |
| | | AGGCAAAGACTTCCATTCTGTTGGTGGTAATCCTATCGATTCCTTAATGAACCCAC |
| | | GACATTGGATATTGATATCGTGCTTAGATATTTGCCACCATATGATGTATATAATT |
| | | AAAATACATATGCTTAAGGCGATAGTATTTACTCCCTGTACGCGCAGTTACCGTTG |
| | | GCATGTAACAATTTAATGGCCCAATGAAGCGACTACGAACCATATAATTTGCTACA |
| | | ATAGTACTATTAACATGCTATGAATTTATGCAAAAAAAAA |
| 84 | 38.80% | GAGTTGATTTTCCGCATTTCATGGAAATATAATAGGGTAACGTTTAGTTACGGAAC |
| | | GTATTCTTTTGAAAACTCTACTTAGTGTCGCAACTAAACTTCTCTGTTTTAGTACA |
| | | GTCAGGATTAGAGACTACTAAGAAATTCCTGATCTGCTCGCTACTGCCACACTTTA |
| | | CGCAGGAGGCTTGTTTTCGCAGTAACCGGTGAGTTAAGGTCCAACAGGGTCAGATG |
| | | TCCCTTTTGTCACCACGAATCACTGGCTCATTAGAAATTGATAGATTTGTTAAAAC |
| | | GAACCTCTATGTCAACAAATGCTTGGAACGTCATTATGACAGTGTTTTGATGTCAG |
| | | TTTATCCAGAAGGGCGAGAGGGTCATGGCGCGGTCAATTAGAGGTTCGCATATTAG |
| | | TACTTAGGTATTGTCAGATCACCGGAGTTTGGAAACCCTGCTTGTGTGATACCTAC |
| | | AACTTAACTTGGCCCAACATGAGAACGTTCCATGCTTCTGGTATCCGTGTTTAAGC |
| | | TCTCAGTGGAGAAATTCTTAAAATGATATTCGTAACTAAAGGCATGAAACAAAATG |
| | | TGAGGATCGGTTATAATGGACACAGTCCTGACCCCTTCGATTGACCTAAAATATTG |
| | | AAACTACATTCAAGTAGCGAGAATTTTTTAATTGTTCCTAAAGTTTTATTATTAGA |
| | | TAAGTGGTCGATGTGTAGGAAATAAGAGATGATAAGAAAACCAGACGTTATTTAAA |
| | | GGGAAATGTCCACCAGTGCCCCAGCGTTATAACATGATAGCCAAGAATTTGGTTAT |
| | | ACGCAAAGTTCGATTGCGTGCTCGGTTACTGGAGATCAAATTAATGGAGCTTCAAT |
| | | AATAGTACTAAATCATGTTTTCAATTTCTTAGCACATCCCCACTAATAGTTTGTCT |
| | | CAGATATTATATGATATAGTTGATCGACCCTGTTATACGCCTAAAACCAATTCTCT |
| | | TTCGCTACCCGAGAGTGAAAACATATTCAAAGTTGTCAGCCTCGACGTTTAATCTT |
| | | CGTAATAATTTGTCGGTAACAGATTAAATACGGAAGACAAATATTATTATCTTCAA |
| | | CTGTCCAAATTCTCCGTCTCCATTTGAGACTTACTCATACTTCAGTGACCTTGGCA |
| | | CTATAGCTGATGTTTGGAGAGAATTAAACCGAGATACTTATAATAATGAGAGCTAA |
| | | TGAAATGGTAGTTCGTATATGCGGTTATAGACTGTAAGAACTATCCAACAGACTCT |
| | | GCCGCACTCTCAGATTTCATCTTAGGCTAGGTTATAATGTATGGGACGGCTCGGAT |
| | | ATTCTATTGAATTTAACAATTTCGTCCAACAACCCTTGGTAACTGAGTTTCCCGAT |
| | | TACATGACGATCCAGCTTACCGTAACCATAGAACTTGGCAATCCTCTCCTTAAGGC |
| | | GCATGACTAGATCATCAATCGCACTTCTTCAATCAAGTTCTCTATCTGGCGCGGAC |
| | | ATACTGTTTTACGTCTCGTTTCATTGTAAAAACCCTTCTGTGTAATAAGAACACGC |
| | | GACTTTGATGGTTGCGATCCCTACGTAACGTGCACTTAACTACATATACTTGGTGA |
| | | GATTGTGCTCCATATTGAAAGTCGATGTTAATCAAGACGGAGTTGTGATTAATAAA |
| | | ATGGCATAATACACCTGTGTTTTTCCTATATAATCCAGAGAGGAAAATAACTGTTT |
| | | TCCGACCAAGTTTGTACTAGATTTATGATTTTCCGAATATGCATCTGCGTGAGTGT |
| | | GTACGTCTGTGTGCATACGTCATTCAGAAAGATCTTCCGTATGTGAGACCTTTTGG |
| | | ATCAGTTGTTCATTTTTGTACCTGCCTACTTTAGACCAGGTTCTAAAAGGCTCATT |
| | | TAACACATGATTATTATAGATCATATAACCATTACTCCTAATCAAATTTGTGCCAT |
| | | CGTTGCAACCGAAATCGTCTAGCAAGATGATCATCGAGCAATACCGACCCTTTATA |
| | | TAGGCTCAACCCTATATTCAGAGGAAAATCACGGTTTGTC |
| 85 | 38.90% | GTCCATCATTGACTCTGTTTTCTCGAGGAACTCTGCAAACCAGATAAGAGATTATT |
| | | AGCATATATGTACCTAGAAGGACATATTATCGTGGACATCCCGGGTGTTTGCTATT |
| | | TGAGATTTATTGATTGTTTTTTGGTAAAAGATCTGATTTACATGGCATTATAGCCG |
| | | AGGCTCATGTTTACATTAGCATAGTAGGCTGGACTAGTTGCGAGAGATTTTGTTAC |
| | | CCGGGATCAATTGCCATTACATCAAATCACGTGAAACGCTTTTCCAATACATGCAT |
| | | ATCCCAGCCGATACTTAGTACGAGATGATAGTTGTACGACGGATATATAATTACGT |
| | | CTATACGTTATAAATTGTCACCTGTCACCACTTTCTGAATTAAAAGCTGAGGGACG |
| | | AGCCGTATTAATACTAAGAGCGTAAGAGCCTCCTAGGGTTATATAACTTCCGCACT |
| | | CAGCTATTATTATTGAACCTGCGTACAAGTATCTACTTATTCAAGTTACTACGTAT |
| | | GAATTAGTAAGCATCTTGTTTTACTTATGACCGCAATTTCATACGTTGCATGATAA |
| | | GACAAGTTCAAGCACAATAACTACGGCAGTAGGAATTGTGGCTCGACAAGAGAGAG |
| | | CTGTTTTCGCCGTTCTGGGGATGAGCATATTTAAAGTTGTTTAACACATCCTTTAA |
| | | CGATAACAAAAGACATACACAGGATGAGGTATTTCTGTCAAGAGAATTGGTAGTTT |
| | | GTGTTAAGAAGATCCCTGACCGTCCTTAGATGGAAGAATTAACGTCCATAGCTGGA |
| | | GGTGTTGTCTTTATTCACGGAAGCATAAGAGACTCGTAGTACAGAATAAGACGGTC |
| | | TCAGGGTATCCACCAGGATCAACGCCAGAAAGTGGGCAACAGATCGGAAGTGGAAT |
| | | TCGGAACAAACTTCATATGTGAAAGAAAAGCTTTGATACGACTTCCATGCCTTGGT |
| | | GATAGGTCAAATTTAGCTATTAGAAACTGCAATGGGAGATGTTCGTGCATGGGAAG |
| | | TAAATGTATCGACCATAATCGCTCTGCGGGCTAGAGCTTGCGGACAGTTAGCGGTT |
| | | CTTTAGACGGGCTGAACCCTATCGAGAACCGATACAGCAATGTAGTCCATTACGAC |
| | | ATATGTGCTTCCTCGACTTTACTGGAGAACCTTAAGACGCGATGGATTATTTAACT |
| | | AAATTTCCAGTTATCTGAACTGGCATAATTTACAACAAACCTAAACATTTTCCATA |
| | | GAAACTCGTTATGAGCATTTCATGCAGTGCGTCCACTGTGATATCTGTAATGGTAA |
| | | TCGGTCCTCATGCGATACGGCTCGGTAGTTTGTCTTGCGACTTAAGGCAATGATGT |
| | | GTGGCATGCTGTCCAGAAGCAGATAGATCAGGGTCAAGTATTGCCCGCCCATTTAA |
| | | TTACTAAAGAGAATAATGCACATAATAATCTCTATTGTTAATGATATAATTATTCT |
| | | AGTGATTTATATCTTTATAAGGTAAGCGATTTCAACAAATTAAATTAAACGCCATA |
| | | AATTTCTAGCAATTTAGATACTGTATGGGACTATTAGGGACTCCATAATTAACGTA |
| | | TGACATACTACACTAATAACTAAACTCTATTTGACAGTTGCATTGCTTAAACACCC |
| | | TTGTGTGTTAAACCATACAACCTTATGTCTGGCTATATTTGTACTTCAGGACCGGG |
| | | ATTCATGATAAGTGCTTAGGAACCTAGACGATGAATCAAGATCAACGTCTTATTTA |
| | | TAAAACGTTGACACAATATTAATCCTACAAGATCTAACTTTACCATTAAACAGAAC |
| | | TTGCTAATCCCTAATGACCAACAGACTTCTGGCAACGAGAAAAAAATAATCATAAT |
| | | TTGTGCGGTACACTTTAGCATTAATTTCTAGGATTCAGCTAGCTGGGCCTAGGGAA |
| | | CACGAGCTTTACGTGGCGTCGTCCGAATCGTTAGAGAAACATTGTGAGATACTCGA |
| | | TATTTTTATCGGTAGAATCCTCCCTCATTCTTACAATGTA |
| 86 | 38.70% | CTCAACAGCATTCTATAGCCACTAATCTTATCTCACAGGCGCATTGCTGCCATACC |
| | | GTTAGAGGGTTTATGAGTGTGGTGCCAAATTTAATTTCCAGCTATTGCTGAGAAGT |
| | | CATATAAGTTTAAGTGCCTCTATTCATGAATCTACGAAGACTACGCCGTCTGCGCA |
| | | CTGGCTTTGCCGTCCCACTTAATTTAACGTTAATATGCAGGTCCGGGTTAATTCAT |
| | | GAAATTTATACGAGGGGGTAGATTGTCGCATTATACGCTCACCTACAAATCTGCCT |
| | | ATCAGCACAGCCATTATGACTAGATTTACCGGGGAATTTTCATATACACAAACCAC |
| | | ACTCATTTTCCCACTTATAGGATTGAGTCTCAGATCACACTTGTGCTGCTTGCTGC |
| | | AAATCCTTTTATCATTGTTCATGGTTACTTGTTTAACTAATATCATTCATTTAAGA |
| | | TAGGGTATCTTTATACCTTGAGGCCAAGTTTTTTCACAGAATACTGAACATCGAAA |
| | | CCTTTACTTCAAATAGATCAGGTAAGATTGTTTTTCATTTAAAGCGATTCGCTCAT |
| | | ACAGCTTTCTGTTAATAGTGATATGGATTGGAAACTAAATTACCGAGATATATCGT |
| | | CATCGTCGGCAAGCAGCTGCTTTATACTAGGATACAGAAGACGGCCGTTTCCAGTA |
| | | AAAAAACCGCCGATTCGATCTTCGATTATTACCTTTTTACTTGCGGCACCAAATGT |
| | | AGCTGAATTATGTTATGAGCTATGCGTAGTATACCCCCTTTGTCCTAGTGCTAGGC |
| | | TCTATCATTTTATGAAATTTAACTCTTGCTCCAGGATACGTCGGATGTACTTTTAA |
| | | CAAAATCTACTGAGAGGACAGGATTGACCACGTAATAGTAGAACTGATAGGCGGGA |
| | | TGATAGGATCATGGGCAGTATTGCTGATTTTAGACCTTGGAGATAGCTGCTTAATG |
| | | AGCTCCTCGACCTCACACTTACTGCAAGGTCAAGATAAGAAAATCTCCTAAAGATC |
| | | AAACCATTCCAAATTCGTGTTTACATAAATTTTACTATTATACATCGTAATGTTAA |
| | | GTGATTTAGCTACTGTGTGTCTAGGATCCAGGATAGTCGTCTAAGAAGCCGACCAA |
| | | CGTGCTAAATAGGATTTGAACAGCGTTATAGTTTAGTTTATAAGGTTGTCTATTTT |
| | | ATCAGTTACTGCACGACACATATACTCTCAGAGAATAGGGTATCACGGTATACATC |
| | | GCTATCATATTGACTAACGATTGTTCACGGCTTATATTTTCACGAGCATTCCAATG |
| | | TGGTAACCATTCGCAATCATCTGGGCTCTCAGTTGTTAATGTAGAATTTAACCAGG |
| | | TTCCGTATTAGTCGAAATCGATGCTCTATGACCTCAACCTTCCTCTTGTCATGATA |
| | | GGGTGACTAAAGAAGTTTCCGATACGCGACGTGAAGTCCGATTATTATCCAGATGG |
| | | TAAAGTGAAGCTTAAAACATAAGAGATCATTCTCTCTGATGAGACATAATGATATC |
| | | ATTTCAAAGTTCTGTTAATAATACAACTGCTAGTCAACGGAATCCTTTCCATCTAA |
| | | AGGCGAACACTAACTAATTTGAATGAGAAAGATAACACTAAAACCGCCAACCTAGT |
| | | AGTTACTTGAGCTAACACATATATTACTTAAGTAGCTTTATCTCTGGTCTAAGTCG |
| | | GAGGTCACAATGACTTGGACTTCTTTTAGTTTTTCGAGTACAACTAGACAATGACC |
| | | TCCCGACGTAGCATATAGAAAGTTAGAACATAGGATTACCGAGTGGTAATAGCCCA |
| | | ATCAAATTATGGTGCGAAAAGATAGTACTGTACTCATTACTTCCGGTATGGGACAA |
| | | AGCCGATCTATTTGTCGGAGCACGTTAATTTTATGACCGGCTACCCTACGTTTACT |
| | | GAGTCTAAAAATTTGTAAATACAAAAATTTTTCCCGCGCTAAGTTAACCATAACTC |
| | | TCAAGTTATACGGGGTAATGGATCTTAAGTTCCCGGAAAA |
| 87 | 39.70% | GTAAGACTGATTAAGAAATTACATAGGGACCTGGAACCGGTATCAGATTTCAAATT |
| | | TTGGATAATAAACCGCCAGGTGTTAACCCATCAACATCTAGTATTGGCGTAGTGAG |
| | | ATCTCTTGCATTTCAGACATCCTGGGACGGCAGGAGTTTCTATCCATTTTCCGCAA |
| | | GTGTTATGCTCCAATTGACAGATATGTCGCCGAGGAACACCAATCTGGAGAATATT |
| | | TAGTCGAGAGGCACAACTGGTGTTATAATCTTAGTGTTATCAAGATGACCTTTTGG |
| | | AGTCCTTTGGATACATGAACCCATACAAATTATCAGCGCTCTACTCTTCTGTAACA |
| | | CCTCGGAAATACACTGAAACAGATGTCAGAGATAACCATGAGTGGTGATTGCAATC |
| | | GGTGACCATGTTCGTAGATCAGTCCTACGAGCGTCCATATGGCGACGAGGGAACTC |
| | | CACCTTTCGAGCAATCATATTGGATTGAGCAAATGGTCATTCAAAAATATACTGTT |
| | | CACTCTGCCAATATAAAAATAGCACTCGTTTTTTCTATTAGGACGATACTAAGTGG |
| | | GCACTTTATCCCTAAATAACTTTCACAAACCCGATTATAGATCCCCCGTATCCAAC |
| | | TGGTAGAAGGCGGCTCGGATCTATCAAGCATTTGCCGAATTTTGCGTGAAATTTTT |
| | | CCACTGACTGCTAAGCATAAACCGATGAAGCCAATCTTGAATGGGTTATCTTGAAA |
| | | ATATTTTGCTAGATTTCATAGAAACTTTGATTAACTATATACGATATACTTATGAA |
| | | TAACGCGAATTACATATATAGACATGTTCTACGTTCCCTGACCTTGCGTCAACAAA |
| | | AATCGGTTATGTCTTAATCAGAATTGTATTATAATACATACGTAGCCGTTTTTTAA |
| | | CTACTGCTTATAAGAGAATATTTCTATACTTACTACACAGATGTTTGGACTATAAA |
| | | TAGAATGACATGGGGGCAGGGGAATATGTATAAATGCCTGTGTGATCTCCAACTGC |
| | | GCATTTTGCCGATGATATGTAGATAATACTTTGAGTCTTGGACGGCCAACGCGCAC |
| | | AGACTACACACTACTATAGACAATGGATGATTTCAGACGCAATAAAATGCTAAAAT |
| | | CCTACCGATTGTCATATTTTTAAGTCTATACCTCACCGTATATTGAATTCATGTCG |
| | | TATCCGAGCGATTTTCGATTTGCCCTGAGACCATAGATAAAACTCACTGAGCTCTA |
| | | ACGTAAGATTCAATTCAATCAATTATAAGAGCAAAAGTGTAACCCGTCGAAGTTAT |
| | | TAAGCTGAAATAGTCGCAAAAACTGTCAGGTATTGCTGTCCAAGTTAGCGGGGCGC |
| | | CATGAGAATGTGAATGACACGGCTCCTTGATATCACAGCGTCAATGTTTAGGTGGA |
| | | TTAGAGCAGAGATATAACGAATGCTCATCCGATATGACGTATAAACAAATGAGTAA |
| | | TGTTAACACTTTTATACTCCGGTACCTCAGTATTCCAGATCTGACGTCCGTGGACA |
| | | CAGTCCTCAATTACGCTGTTATTGTATGGACTACCCATCGCTGCTTGACACGATCT |
| | | TGAATTTATATAGCTACGAATGCAGAGGTTTTGCACCGCTTGGCACTACCGAGTAT |
| | | AAGGATTATGTCAGTCGAGGCCTGAAGCGGGGACTGTGAAAAGCACTCCACACACA |
| | | ACAGCCAATGTAGAGCCTTCGTGTTTGAAATTCTAGGTTTTCAACATAGTTTTTTG |
| | | GCTGCTATTCTATTAACTACTAGCTTTACTTGTAATCTTCGGCTAAAGTAGGAATG |
| | | TATTAATTCGCTCACCGAATATCGCCCATCCTTGACCACGATGTCCCGTCAATTTG |
| | | TAAAAGGCATCTAGTATTCATCACGGTATGGTATCCCTTAAGTTGTGTATGGCTAC |
| | | AAAAAAGTAATGGAATCTAACTAATTCCATCATGCGCGATTCATGAGCTCGTGTCT |
| | | GTATGAAAGAATATACCATTCAATAGACACAACAATGATT |
| 88 | 39.50% | CAAGCTAGTCTAAACTAACAACAGCAGGAGGGCGAGAACGTTGGCCACAAGACATT |
| | | AGGCGTTCTGTTTATCAAGCATCGACGTCTAATAATTTTAATACTAAAATTCGTCA |
| | | CTATCTAGTTGTTCACCATGGATTTTTATGTAGGCGATATCAATTCAGTAAGGTAA |
| | | CCCTAGTTCTCTGGGCTCATGTATGAAATCGGGAAGAAAGATATGAATGAAAAGAA |
| | | CCTAACTACTGAAGGGTAGTCGACGAGAGGCAGCTAATAGGCAACCTTTGTCCCTT |
| | | CGGACGGACTGGTTGCTGAAATTAATTTACATAAATTAATGAAACATCCCCAACGC |
| | | CACCTTACCCATAGGGCGTCTCACGCTATACGGTCTATTTTAATGCCTAAGAATTT |
| | | ACGATGAGCCTATAAATACCTTAGTTGTGAACGAAACGCAGCACACGACAATCGTA |
| | | CAACCTCACTTTTAATGTTATATACGGGCGCGGCTTGGTAAATGCCGTAGCTCTAG |
| | | TAACATAATGCATCCTCACCATACCAGCAAAGCTAAAAATCTTCAAATATTCGTAT |
| | | AAAACTAACCAGTTTAACGTGTATGAGGCGGTCTTTTTACCAGTTTGGGAGCATAT |
| | | TGCACGTACTATCTTCTTTTTAGCAGACCTGGGATCTGAGAACTTCCCCTGGGTAG |
| | | TCTTACGATTATAGTTAGCCTAATAGATTATTTGTTCGTTAGGAAGAATTCATATA |
| | | TACTAGGTTATCCTTCAGGTTGAAAATTAAGGACGTTACAGATTTTTCACAATTAT |
| | | ACCGACTACCATAAGTGGGAGCGCGAATAGCATTTGAGTATTTGGATCAAGCATCT |
| | | GCTGGGTTACACGTATTAATTAGACCCTTGCCGAGATCTAGGGAAACAAAATCCAG |
| | | ACCCGCAGTACGTGGGTGGTATGACGCTTCTTAGGATAGGAGCGCAAGTCCATAGA |
| | | CCTTTATATTACTACGTTTACCTGATCTAAATAATCTGATAGAAAATTAACCAGGA |
| | | GTCCCATTAAGGTATTCAACCACGGAACAGAGTATAATCTGGTTGATAAAGTCGTT |
| | | TTGATCTGTTAAAGATTTGTTAAACTAAACGAGACTTCTTTGGGTAACATCATACA |
| | | AGTCTGATAAAGGATGATGCAGGGACTAGTCTAAAATGAGGGAGTCTTTGGGTATC |
| | | CACCAAATAATTTCAGGAGTTAAGAGCACTTCCAACGATGCAGTCCTTTGGCCTTC |
| | | TCGTGCGACAAGGCAAGAAAAGTTTATAACTCTACAGCTTGTGTAACTCGAAAGCT |
| | | GACCTACTATATAATGTTATTGGAAATCAAACTCAGGGTTATCTTCAAACAGTTTG |
| | | TTATTGGCTAGACAGCTATTACCTTTAATTGGTCCTTAATCTTGCCTATGGACATG |
| | | CTCCACACATTAAACATACTTAATGGCATGCAATTATAGATTGTCCCGTTCATTCA |
| | | CTATAGCTTCATAATGGTTGGGGTAGTACACGCAAAGTCTACTTATATGGGCAACG |
| | | CGCCGGCCCGTCTTTCCTGTTAAGTTACGGGAGGTCGCTAATTACTATTTTACTGG |
| | | GAATGCGCAATCAAATCTTGATTGAGACCAACGCCAGGCCCGAACTATTCTTATTG |
| | | TTCCAGAGTCTTTACTTGAATGCATAGTATCGGGATGGGGTGATGCCGGCCACCGG |
| | | ATCACCATGGATATACGTCAGTTGGCCCACGTGTTAATTAATGTCATATTGTTATG |
| | | GGCTAATACATTACTGTATTGTTTAAATACAATTCGTCATGCATTATCAGTACTGT |
| | | GTAATTTATATAAGCGTTCATCATTGAACGTGTATTTTGTTGGTGCGTACTGAGTT |
| | | AGATATTGGAGAAATTCCCTAACCAAGGAACAATGACTGGACTTGTTAGCGATGTA |
| | | AGAGTAATGCAAAAGTTAATGAGACTGATATTGGAAACAGTATTGTTTAGGCTAGT |
| | | CTAGAAATAAACTGCTCATAAAGAATCTTGCAGTTAATAT |
| 89 | 39.60% | TTCACTATTAAGTACACCTAGTCAGACGTGAAAGTTAGTTCTTTTCACGTCTCATA |
| | | TAGTGCTATTTTCGACCACGTCTTGCAATCGTGATAGACAGAGCTGTCATTAACAA |
| | | GATCAAGTTATAAAATTGTACGGGTTGTACCTGCTTATAGTTATATGTTGAAATTG |
| | | CAAGGCCGCGTTGTGACCGGTTTGACGGAATCTGAAGGGATTAGAGGAGTTTATAT |
| | | TTAATTTCTTTCATGTAGAGATAGAACCCAATAACCTCTCGCTACATAGAACTAAC |
| | | GTTTTCGCAGTGATTTACCTTGTGAAGTGCACAGTACACTTCACTGCCTTTTACTC |
| | | GCATATTGATACAGTAGCCAAAAGTATCATTATTAGTGCATAACCTTCACCTATTC |
| | | CAACGGTTTTACGCATTCTGCGTACGTTCGATTGAAATAGAACAAATATAACTATA |
| | | ATTGGTACCCATGATGTAACATTTTACCTCAGTAATATGTCGAAGATAGGCTAAGT |
| | | CCCCAGCTAGCGTAACTAGCTAAGCCTTGATGCGTATTCCTTAATCTTGTTTAACG |
| | | TCTCTGCTTACGCTAGTTTTTAGTAGAGCATAAGATAGCAATTTCAGGATGGAACG |
| | | AGTTATAGAACAGACCACTCCTACAGTGAGTAGGGTCACATGTATTGTCCGACACT |
| | | GTTTATTCAATTCCAATCTTTTAAGTGCGAATATAATAAGAAGCACCCTTTCAAAC |
| | | AATTGTTATAATACGTTTTCATGACACCAACGATGTCGACTATGATGTGCTTCTCT |
| | | TTTGGTTAGACATCTTTGCATTTCGACGACTCCTTTTCATTGAGCAGGTTTTAGTT |
| | | AGCTAAGTGTTTCCTACATTGTAGCGCATTAGTCTAATAGAGAGTGAGCATTAGTC |
| | | ACAATATAGTCCAATGGATCTGAGAAGCCTTATGAGGCGTGCTTAGGGAACAATTG |
| | | CAGTTTAGGCAGAAAGAGTTACCCTTTAAGGGTGGTATTCTTATCTCATATCTATC |
| | | TTATTGGTGCAAAGTTTGTCTTTGAACGACAGAGTAACTCCATTCGCAGCCTTGCT |
| | | AAAAGTGGAGAGACGCAAAAGTGGAGGCACAGGTCGTTTCTTTTAGTCGTATATCC |
| | | AGTTTATGAGCTTCACATTTAAGATCAAATCCCTTCTCGAAATAAAAAGGATTCCC |
| | | ACTTTAAATAGGCGATTGATTGTGCGCACTATTTATTCGTAATCTATACGTAAAGA |
| | | AACTGAACGCCACAGCCTAATACATGCTAGTATTTCATACATGTGAGCCGAAGACA |
| | | CGCACTTCCTTTTTGATGCGAGAATTTAGGGCGACCAAGTCTGGTAACATTCTGTC |
| | | CTAGTTGCCGAGTAACATAGATATAAGCCTTAGCAGGGCGCGGCTATACCTTGGTA |
| | | GTAAGACGGGTGTTTGAGTAATATTAGTAGCTTAATTAACAGCGGTCAATCGCCAA |
| | | ACGGAATTGTAACTGGAATGTCGTATAATCCCATTTATATCTCAGCACATAAATCA |
| | | AAATGGCTGTGAGATTTAAAGAGGTTAGTAATTGTTCAGAAATCCGAAATCCTCAT |
| | | AACCAAATAAAATTCGCATATGCATACTTGATCGGCGGAGCGATGAAAGAATTACA |
| | | CTTTTAGTATCCAATTATAAACATCATTTGCGGCCTACTTTTCCCAGTAAATCAAT |
| | | ACGTGGAGAACTGGCTCGTACTCTGCTCTACACTTATTGAATGAGTTAGCCAATGT |
| | | AGAGCTGGATACTAAGCTCTAGAAGTTACTCCAGAACAATTACCACGTTAATAACT |
| | | TCTATTATTCAGAGTCGTAACAGCCCTCAAGTCCTCTCTTGTTCGCCTGTCAGCAA |
| | | TCTCCTACGGACCTACCCTGCCAGGTAGTTGCTGTCTAAGCCACTATTAGAGTTGC |
| | | TAGATTTGTTAATTATAATGCTTCGCCATAGTCATCCACGGTCAGGGCGGTACCTC |
| | | GCAGCTTGTGTAAGGGATCCCTCGAGTAACTCTTGATGAT |
| 90 | 39.60% | CGTAGTATTTTGTGAGCTAGATGGAGTACTCCGATTCAAGGTATTATGAACGATAG |
| | | ATACCGTGGCTATATCATAGGATTGCTACACTGTAGGTTCCAGACCTTAGCGAAGC |
| | | GGATACCTTCCGTTCGGTTATCTGTTAAAAACTTTACATCTTCATGATAAAGTGTG |
| | | CCTACCTTTGTATCACTGATGTACTTCCCTACAATAGATACTCTTTAAGACCTGAG |
| | | TACGCCGAAAGAATCTGTTCGATCTAGCAACGACAAAACAGTTATCAGCATATCCG |
| | | TATATTGTGGTGTAGCGTCTTCGTGTACTAATTTAGATTTCTGCATCTGTCTAGTT |
| | | ACGTGTAGGGCCTATGACGGTCCCTTGCTTTTCCCGGGAAATATCAATTGCAGTTG |
| | | TGAAAATTGTTTATAGGAAAACACAAATCTAAATAAATTACTCCAAGGATCTTCTC |
| | | CCAGATGACTATTCTTAGATAATGAGAAAGGGAGACTCGATTAAGTAATATTGTCG |
| | | AGCACCACAATCTGCCTATATTCTAACTTAGTAATAATTAATTAATTATGAGTCAA |
| | | CCAAAGGGTCGTTTAGCTGATTCATATACATACTATATTTGATCACCACCTACGAG |
| | | CAGTTGGCATAATTTCCTTGTTGACTAGTTTTGACCCACGTGATTCCCCTAAATTT |
| | | TTTGTGCTCTATGACCGACAACCACAGTGTAATGTCTCAGGTAAAAATGAGTACAT |
| | | ACTACTTTTCCAGATTGCATAAGTTATAGACTTCGGTATTTTCCAAATATTATTGC |
| | | ATTGTACTACAAAACTAACGGGTATGAGTAGACACAAACGATCACGGGTTTCACTT |
| | | ATGAATAACGTTGTAACGATAAGTGCGCCTCGCCTGCACCGCATCACTAACGCCTT |
| | | TTTCGAGGTAATACCACGTTCCGAAGAATCTATTTAGTTCCTCGAATAAAACATTA |
| | | TTGATAAGTAGTGAATCACCAGCCTCCCAAAAATACCAGAAGAGAGAAACAGGTCT |
| | | TTCAATTGCTGGTACTATTTGATATCCTTTACACGTTTTCTATTCTCCAGTGTAAG |
| | | TCTCGTTATGCAAGTTTGTCAATATCAGAACAATATGATATACAACACCTCGCAAG |
| | | CTGCTAGCAGTTAGATGCGATCCGATGATGATCGATAAAAACTTATGTACTGGACC |
| | | TGCTGGTTTAGCCTTTAAGAATAAGTTGATTCTTGACATACAGCTCGGGCGATAGG |
| | | ATTGAAGAGTAAAAGCGATGTAAACCAGGTCTGTGTTCGATGCAGAGCAAGTTCCT |
| | | GCATCGGATTTTTCGGATATGCAGCTTAGATGGTTACTCAAATCCAATTCCGGGCT |
| | | GTTGTCTGTACAATTTGGGAGGTTGACATTGCCACCTGGGCAAATGTTGTCCGAGA |
| | | ATTCGCCCGATGAGAGAAGGGACTTGGTGGAGTCACAAGAATAGGCGATTTCGCCC |
| | | CAAATTTAATATCCAAAAGAAGGCGTTCTACTAACCGTAACGTTAGACATATTCGT |
| | | ACAGTGAAGTTCGCACTATGTGTGCATTACTCAAGTATCTGTTGTATAGGATACCT |
| | | TAGTGGTTCAGTATTAAACACGATTCTTTTATCTTGTATGTTGTAATAGCGATCGT |
| | | TACTTATCAACAGAGTTAAACCATGGTACAAGTGCACAAGTCATTAAGCATCTAGA |
| | | CTGCACTACATCGCTTCTATATTCACCATATGACGTTACAATCTCCCAAAGTAAGT |
| | | ATGTGACAACTTCTCCGGCCAGCTACATCCGGTAGAATTGTGTTAACTAACAGTGT |
| | | AATTATACTCCATCATACGATTTAACCGGTTGAATGACTAAAACTTAAGTAGTTCT |
| | | CGCATGGGTCTCCGCCTCACTGGTAATATGTGACCGCTCTATTGAATTCGAGACCA |
| | | GGATCAATTACATCCTCACCGGGTAAAGAGTAGATCAGGATTTTTAAGTGAGTAAC |
| | | CTGGCGATGAATACAAGGTTGTACTGCAGTTTTACCCTGA |
| 91 | 39.20% | GATTTAAATGGTAATTAAAATCGAAGGTTTTAAAAGGTGAGAATTTTTTTATAAAA |
| | | TGCAATCTGTTACGCCCCTAATATTCGGTTTCATGATTTGCTTAATATTGTATCAA |
| | | CACAAGCATATTGTTAAACAGTCTCTGTACTTTCTTGATGACCAATAATGAACAGA |
| | | TGAAGTCTTCATATATTGAACTTCAATTGAATGCGTGCATGCCATTATTCGTCATC |
| | | GAGAATTAGGAAGAAAACAATTGCAGCCTTCTAGCGCCAATTGCGATTAGTAAGCT |
| | | TCGCCCTCACGTACTAAATTATATTAGACTGATCGGAGACATTAACAAGCTGCTTA |
| | | TTCCGTCTTGAAGACCGTATTTCTTACTGTTACGGTGTCCTTAGGCGTCATATATC |
| | | AACTAATATAAACCGGTACTTTATTCATAATAGCCGATATTCAGTGATTGTTTGCC |
| | | ATAGGCTACTTTCTTTCCCAAATCCCCGGTATCGCTATCCTATGATTTCTGCGTCA |
| | | GGGGTTAATTACGGCGACACCAGCCTAACCCAAGATCAGACTAGGATAATATTTCA |
| | | CTGGCAATACTCATCGATTAATTCAACTAGTATCTATTTTTTCACACTCCGCAAAA |
| | | AAGGGCAAAACAAAGTCGTCAAGCCGGGAATAAGGGTTATTCTTGCAGTCTTCGTA |
| | | ATAAAATTTGAACTCAGTTATTGCGAATTTACTCGTATAAAGCTTCTATTATCATT |
| | | CTCTGATTACTCAAAAACGCTCCATGAGGGTAGTAGCACATAAGTAGAATTGCTCA |
| | | TAGTGGCTTCTTTCTCTCAATCCCTTTGATACTCATTTTTATATTACTTACATGTA |
| | | ACGATTGTTGAAGGCCAGCAAACCATATAAGTGGACAGAACAGGGAACAAGAGAAA |
| | | ATAATACAGAAAGTAGTAACTAGTCAAGAAAGTCTAGATGAATCTATAAGTTGTAC |
| | | CTATCGAACTATGATCGTAGCATTTTCAGTCTACTTGAGGGAGAGGCTGTAAGGAA |
| | | TTTTAGCGGCCAGATATATATCGCTGGAACCAAGTTATCGCATGGAAACTTGATCA |
| | | CGTACAGAATGTGATGTACGCGCAAATTAGATCTGAAATCCCTCTGTCCTCATTTT |
| | | TTAATTAATACAATTAATATCAAAGGCCTTCTTTTCTGAATGTTATTAGACGGAAC |
| | | ACGGAACTGCGATTCATCATCCTAACTACACAACACGAACTGACCAGATTTGCGTG |
| | | TAATCGTCACGTGCCGTTGCTTACTCTAGTAAACCCCGGCGCAAGGGCGAATTGTG |
| | | AAAAAATGAGTCAATTCGCTACAGTGGCAAAAAACGAGCTCCTGGACGACACAACC |
| | | TCGTATAGCAAGGCGTAGCTCAATGCGCCAGATATTCAGGTATTGTAGCCCATGAC |
| | | AACAAGAAATAAAGCTATAGTAGGCATCATTATCGTTTCGTCCGGCAGCTTTTTTC |
| | | TGACTTCCACCTCATTGCGTCTTATGTCATTACTGCGTAGGGTCACCTATATGAGT |
| | | CTTCATCCCTGGGACACTGAAGGGAGTACGCCAGTATTTCATCTATGAATAAACCT |
| | | CGATTACTCCTTTATGAGAACAATACTTACACTCGACGGGGTCTTGTGGTAGTGAT |
| | | CTTAAGATTATCTACCATTTGTTCACCCTTGAAAAAAGAGACTTACCTCTCGACTT |
| | | TTTTCTATACTGGGCCCCGACCGCTGACATGCAGAATATTGAGGAGATGCAGATTG |
| | | ATATTTACAAAAATTAAAGCAGATACTCAACGCATATTCTATGAAAATCAGGGACA |
| | | CCCAGGGTGGTGCTTTAGGATGATTTACATGAAACTTTAAAAGGACCGGGATAAAC |
| | | TGGCCGCCGGTCTTTCACTGCCACAGGGATCTTATTCATTCGGATATATTATTGCC |
| | | ACTCAAGATAAATTCTGTTAGTAAGTGTTAAAGTGTATCATTATTGCCCATTCTTC |
| | | AGACTCGAGAACTTCGAAGGCAAATGCTGGACGTGTGTAC |
| 92 | 38.70% | AGATCCACGGCCCTGAAATCGCCATCGCTGTTCTTCTTTGATGAATAATGCAAGGG |
| | | CTGAGTTCATCAGTGTATTCGAATGCTACTATATTTCAGTATTGTGAGTATCACAG |
| | | CTGTAATCTTCGGAAATACAAGGATGTTTGTCGACCTCGCTAACACTAGATTATTT |
| | | TGGCCCGTTACTATTTATATTTTTATGACTTCAAAATGCGCTTCAAGATTGTAACT |
| | | CTGGTTGATATAGGATGCAGGGACCGGCTCAGGGCCGCTCTGCACTACATTAATAC |
| | | CTCAGGGATCTCTATTTCGTTAGAGCACACGACTTAGTGACTAGAATAGCTTTAAA |
| | | TGTAAAACTTCATCATATATTCCTCCTGGCTAAGCCTTAATTTCATTCTTGGGGCT |
| | | GTTGCCAAGACTGCTCAAGAGTTAGTTTTTCTTTCTCCTTGTAGTACCCGTTCTCC |
| | | TAAGTGCAAATAATCTATACACACTTCATATTGGGTATACCATTCTTGGTTTATTG |
| | | TCACCTGTTATGTATTTTGCATCAAAATAATCATCGATGTATACGTTAACCCAGGA |
| | | GACAATCGACCGGCTAATTCCGGGAACGTAGATGTATGTAAAGTAACATGTATTTC |
| | | AATTTCTTCTGAAGTATGAGATTTCAGTTGCACAAAAGGTACTCAGCATGTCTTAT |
| | | CATCCATAGGGCCGCAATTATAGAGGATCTTGAGTGGAGGGTCCATACGAGGCCTT |
| | | AGGAAGCCGGCTTATCTCAGCGAAGGTTATCGAGATGCTAAATTTACGGATAAAGA |
| | | TCCGTTACTCTTCTTTAGAACTACCGTTCCAACTCGAACATAGAATCGGCTCCGAA |
| | | TTCTTGGGTACCTTGCAGAACTGAAAAATAGATATCTCGGTATCTAAAGGCAGAAA |
| | | TAGTTTTCGCTCTGGATTGGTTTCTAAAGTGAATCTCAAGTTCTAGGTAAGCATTC |
| | | AAGTCCATTGGGGACCATTAGGGGTTAATACGCACTGACGTCGGTCTTTCGATTGA |
| | | TAAATACTTAACCTCGTTAGCAGTGAGGGTCAACAATCATTAATCTCCAGCTATAG |
| | | AGCGGGTTAGCCAGATTTTATATCGGCGTCATTCCTTTTATCTTTGAAATTTAGGC |
| | | CAAAAAGAAGGGAACTGGTTCTATTCGCGAATTGAACCGCATTTATGGTAATAGAT |
| | | CTGACCACGTGCTACTGCTCACTTACAATAGCTAGTTTTCGGCTCAAACTTTGTAT |
| | | AAGGCTCACTAGGCATATAACGAGTTAAAACTTTTCACATGATACGTGACTAGCTT |
| | | CGCCCGACATACTATATATAAGGTCTACCGTTGCGGGAAAAGATGAAGATGATATT |
| | | ATCAAGTCTTTGACTAATAAATTAACTTATGCTTACAAATTTCCAAAATAGATATT |
| | | CCAGTCGTCTATCCTTCTATTACAGAGAAAGGCAGACTTAATCCGTTCATTATATA |
| | | ATTTATTTAGATGTTAGTCTTTCTGGTGGGTCGATTGTTAGTCTTTACATAGAACT |
| | | CCTTTAATGTTCATAAGTTTCCATCAGTAGAAAGTGAGCTTATGGGTTATTCACCT |
| | | TTGATATTAAAAGATTTACTACTGCTATAATCTACCTAGCTCAGCTGAGAGGCAAG |
| | | AGGATCACATGTTATTGTTATAATGCTTTGATTGGTAAACTATAGTGTCAAGGCAA |
| | | TTCGAGTGTCGCCAAGTTACGTCGATTAGATCGATCATTAAAATCTAATAATGTTT |
| | | AGAGTTTGTTAGAGTAATGGTGTTGATCGGCACATAAGAGTCAGAACGCGGGAGTA |
| | | TTGATATTTTGCCGAATTGCAAATTTATCAACATCGGTTCTACGTATCGTTGATGT |
| | | CCTAAGGCCTTAGTTACGTAGCTTACATTTAATGCGCATAGGGTTGAAGCGTGTGT |
| | | TAATCGCTCTTTCAAATAAGTGTTAGGAAATATACGAAGTAACGAATATCAGCCTA |
| | | ATTCCAGCGACTAAAATGAAACAAGAGCATCCGGTGGTAG |
| 93 | 39.50% | TTGATAGTGTGATTAATTAGCTGGTCATTATCGGTATCGTTGACAACAGTAGGATG |
| | | ATGGCGATTGTCTGCAGATTTCGTCCATTAATATAAGTAATACTTGTTATGATGTC |
| | | CAACTTAGATATATTGGAGTTTTATTGCTCTATTTCCTGTACCCTTGTGACGAGTA |
| | | ACTGCTCCGTGATATAGGCAAGTTAAGTGTGTCGCAATATGGCAGTAGGCTGAATA |
| | | CCACACATACTGTCTTTCTAAATAACACTAGGCGACTACCTTTAACTTCATCTAAG |
| | | GACGTTATTTCACACTAAGCACTCCGTCCCGAGAACAGGGTCTATTGAGGCTACTG |
| | | ATTGCGTAAAGTAGTTGGACACGCATGGGTTCTAGATCCTCATCTCTGGTTTCTCA |
| | | ACATATTGAGTTATACTTTCTGTTAGTTGTTAAGCCGGGCGATCAAAGCATTTCTA |
| | | CTTCAGAAATGGAGGACTGTAGTTATATACTACATTCTGAAGCGGTACCATTAATG |
| | | CTTTCCGCATTGATGAATATCTATATTTACAGTTTGGTGAACACAATTAGGAGAGT |
| | | CGGACTGCGCAAACAGAATATTTAGTTACTTATAGTTAATATACACCTATACACGG |
| | | TAGAAGGTCAGTTCATATAGACTTCTGGGTGTGTACTTCATCAGAAGTCTCCTGTC |
| | | TGTTTAGCCAATCGCCACCTTCTCAGTCCCGTGGGAGTACCACTCGAATAGATCGT |
| | | TGTTTTCGTTGTTGATAAACGGACCCCGTCTTATTTTCGTTACCATTTAATACGAT |
| | | ATCATATAATTGAAATATTAGGAAACGGCATTTCAAATACGAACGATTTGAACTTC |
| | | ACCTACCTTTTGACATTTATATTACAATTTTATAGGGCAAAACGTAATGCACCTAA |
| | | ATTTACTGCACTTCAGATCTACCAATTGATTTGTCACACCAGCTATTTAACGAACA |
| | | ATATGACTAAATATTAGCTGGTATGCAATCTGAAAAGTCAACATGGTATTTCTGCT |
| | | TACACCGGTAGGGTTAATGGAAGTTCTGCGCCCATTCGAATTTTAGAACTGAACAA |
| | | TAATTCATGAAAATTTACGTTAGCAGTACCTTTTTGTCTTACTAGTTGTTGCAGAA |
| | | ATTTAAACATTACTTGGTAGCCTGCTGTGTATATAAAAGAGCGATCTCCGATAAGT |
| | | TGTTAATCTGTTGCTACCTAAGCGCTTACTGTGTGCCTTGGCTCGCGTATATGCCC |
| | | AGGTCAACATTTATTTGTCGCTCGACTCGAAATAATCTATATCATAAGATGGGAAC |
| | | GAGTATGCTCCATGAGGGAGCCGGACTAGGCATTCAATTTTGTTTGAGTCTTTAGT |
| | | AACCATACCTATTCATGCGTAGTTAACTTCGTAGTAAAGCAGCGTTTATACATAAA |
| | | CACCAAAAAATGTCCTAGGGGCATACCAAGAATCTAAGAAACAGCGCAGTAGTTCG |
| | | TTCGGTTTGGCAACCATACGAAAGTATCATTGCACACGACGCATACAGCATCCTAG |
| | | GAGTTTACTATGTCTTCGTTTTTTTGTAGGCCCCACACACATTAAATTCGATTTAT |
| | | TACACTCAGAGTACCTGTCCGCCAATTCACGTGAGTACCTTCGCGCAGCAGATAAT |
| | | ACATTGCTATGCGTTCAGACCATTGTAAGAAAACAGATCATGACTCTAGAAAAAGT |
| | | GGCCTTAGATCAATAAATGTTAAATCCGGTTCTCTCTAACCTCGCCGTACACAGTT |
| | | AAAATCAACGCGCATACATAAACATTGATCTTATGGGGGCTCACATAGTGAGACAA |
| | | TAGTAGTACCCAGTGTTATACCTAATCTAATATATAGGCTAAAAGGTAGATTAATT |
| | | GTCTGATCATAGATCTCAACCGATCATGGATAGCTGGGAATACGTTATAAAGGTAG |
| | | GTCTACGACCCGCGAAATCTCGAGGAACCACAACAGAAACCATTGTCTGTACGAGC |
| | | GACAGCGTATGTACTCCGTGGCTGGTCTACCTCGGTAATG |
| 94 | 39.40% | GGGTAGTTTTTTCTCCAAGGATCCCCTTAACTAGGGTGAAGATTGGGATTAAACCT |
| | | AAGATAAAGATATAACGGTCACTGGCGACAAGCTTACAAATTTGCGCTTTACAACA |
| | | GACCAAGGCGAAAGTAATCTTGGCCCTACTAAACCAAGGGAAATCAGTAGTAGTGT |
| | | TCTCCAAATAGGCAAGGCTAATATCTATACTGTCCCTGCATGATGTGTTAAGCCAT |
| | | AGGCGTGTAATGTTATTCCTTTTCCTAACCAGCTTTTAATGTATCCTTGTGTAGGA |
| | | AGAACTGCGAAGTTATGTTACTCCGAAGCCAACCAACATGTGTCCTCTTGGCACCA |
| | | TGATTCGAAGGTGATATTATAAGTTATTCGACCGTGAAGATTACATATTACTGGAT |
| | | GGTGTATAAATAGACCATACGTTCATTGAAGCGTGACTGAAGCCGACAACGGCTTA |
| | | CGTAATGATTCAAAATCGGTAATAAGGATAACGGTTATATATAGTAGAATTCGAGA |
| | | TGGAAAAACCAACTTGCTAATGACAATATTAAGGGTATATCACACTGTGGTTTGTA |
| | | AAGTAGTCACCTATTCGTGATGCCGTGTACTTCAACTTATAGTAAAAAGTATTGTT |
| | | TTCTAACCAGCGGTAACCTGTTGCAAAAAACCACGTTTAACCGATTGATAGCTTGT |
| | | GGTAAAGTGGCATAGAGTATACTTCCTCCATCTGTAGTACTTAATAGGTGTTCCAG |
| | | TTGCAGTATAAACCTTTCTTCGAGTATCATCACTAAGACCATTAGACATAGGATAT |
| | | ATACAATAAGAGCTGGAACTTGAATCTTCTAATGACAGACTTTACTAATTATAGTT |
| | | CAAGCGCAGTTTAACTATAAATACAATTGTCAATTCATCATATGGTAGGCAAGATT |
| | | CCTTTAGCCTGGCGTACAGTGGCCCGGAGGCCTTGACCAAAACATGGTTCTGTTAT |
| | | ATCACGAGATGGATTGACTATGCTCGTGAATCTGGAGAGGCACTAACTTGGTAACG |
| | | CCCGTACTCTACCGCAGCGGGACAGGTGATAGACTGTCTATGTAAATCGTCATCAA |
| | | TCTATATTTCAATACAACTATAAATCCAGACAAGTATCCTTGAGATAATAGTTAAT |
| | | CTATCCTAACTAATAAGAAGAAAAGAGACGATACGGTAGTAGATTAAGCTTTCGCG |
| | | GAAACAAGAGGAATCTACAGAAAACACCCTAAATAAGCTATTCCATGCCGCCTTTG |
| | | CTATGAACGAAGTACGGAAGCATGATGCTTATCAACGTCAGGAACCTAGCTCAAAT |
| | | CAAGGTCTTACCAGTGACGATAACATGGGTGCGGATGGTTATTTGTGGAGAGGCGT |
| | | AATACAATGTACTTGTTTTCAGGATATCAATTTAATTTCACTTAGAATACGAGACG |
| | | GCCGACAACTTTAACGAATACATTTGCATCCCACATTAATACCTGAGTGCCGCTCA |
| | | TATCGTCCTAGCACAATTTTTAACAGAAGTTTTGGTGGTGAGTAGAACAACAACAT |
| | | GTAGTCATCTTAAGCGTATGAAATCTGGCTCTCAAATTCATGTTTAATAGTGTTTA |
| | | ATCTTTTATGTATAAATCGTTTTTATGGTTTAGACGAAGCACTCAAAAATATAGAC |
| | | TGATGCCTATGACCTGTGCTATCTTTATTTTCCAGGGCAAAGATGATCTTTCCGAG |
| | | TCCATATCTTGAATGACTTCCCGCCTGAACCAATACCTGGTCGGAAGGAGGACTCA |
| | | TTAATAAACATGCATAAATGGCAGATCTGAACTGGACGGCTGACTTATCTCACAAT |
| | | GTGTTCTAAAGTCCACACCGTTTCTGTACCAATGAAAGGACGAATTATACATGCAT |
| | | TGGTTTGGTTAAAACCAATACTTGGTAACGATCTGGACCGGGCGGTTAGAATGATG |
| | | AATTAATGCGCCGTATGTGGAATGAAGTCCTGTTAAAATGCAAAAGGTGGCTCTTC |
| | | GAGAGTTGTTGGGTTGAATGAGAGAAACGCCACCTTCACA |
| 95 | 40.00% | TAGTATCTAGTTTCAGGTGTGCACAGAATAGTTATCCTCCTTTGTCTGTGGCTATT |
| | | TGGAGAACGTATTAGAGGAAGCATATGGCAAAATGGCCTGTACACGATAGATGGTA |
| | | TCATGTTTGGAGGACGCTAGGCATTTCGCCCTAAACACCGCAACGATACCTAAAGA |
| | | GCTCGTCAATGGGCTTGCCGATTAAATACGCAAGTTTTAGTCAGTCCAGACCACAT |
| | | TTACCGGTAATTATGCACAGACAAGATATTATGCTGGTTTATAGCCCATATTTGTC |
| | | TCCCCCTAAAGTGAGCTCTGATATTTGGTTAGGTCGAGTAGTACAGTTTGCTATCT |
| | | ATGGATACGATGTAATTGTGCTTGAGATACGTGCATCACGAACATTGCTAAGCGGA |
| | | TTCGCAATGTTCGTGATGCATGGAGTAGTCTAAGCAATCCAACAAGCGCCTGAATA |
| | | TAATTTTGTCACAAGTAAACCTTCATATTGTCTAACATACAGAGCTGTTTTACCCC |
| | | CTCATGATCTAAATCTTTCGCTTCTTCCCAAACTGCACGCCCTATTCGCCTGTTAG |
| | | CGCATTCAACCCTAATACAGCTGTTGTGGGGATACTCTGATTGAAACAAAGTTCTC |
| | | TATGGAAGCTTCATCATTAGGCCATACGAAATAGAATCCCCTGTTGTCCAGGTGCT |
| | | TCTCGACTGCGTTGCGGTTCTTATTTTGGCTTTGCTAATAGGAACTTCTCTCTTCG |
| | | AGCTCGGTCGAACGCCAGTTCGTCAACTATACCGCCTTCTTTTTGCGCAAGGTCAT |
| | | CGAAACTGAGGTCCATCCTGGGACAAGAGATCAGTTAAGCCTACACTTGTGTGAGA |
| | | CTCCGCAGAAAATCGGGACCAAAGCGTTAGGGCTTCCCAATTATGAGGATCTATGG |
| | | TGTCATTGAAATTGATAATCCTTATAGGGCCATTTTTATCCCTGACCTGAATTCTA |
| | | TTTGGTGAATAAAGTATTGGTCGCCTTTCGAGGGATACTACTATGTTATGGACCTA |
| | | ATGGATGACCATCTGGAACATTAGCAACAGCAACTCTAATCTTATTTTATCATCTT |
| | | CAGTGTAATATATCGTACATTTTAGGCTTTCCTTTATGTTAAATTGTTATTATGAA |
| | | AGAGGTGTATTATAAGCTAGTTAAGCGCGTTAAAACACAAGTGGTCTGCTGTCATT |
| | | CATATACCAAAGAAGGTCTTGATGGACAATGTCTTCACAAGACCATGCATAGATTC |
| | | TAAATCGATATGACACCTAACAAATGCGGGCTAATATTCGATTTCTGACTCCCACA |
| | | CTGTGAGCACGTTTATTGCGGAGACTTTTAAGCGAGATACTCTTACTCCCCATTGC |
| | | CATATATGTAAAATGGACTTCCAATTCTGCATATTTCAGTACATCCGGACTGCGTT |
| | | ATAAGCATTGTCGTGGATGCATCACCATCCCATAGTTCCACTTCTTTTTTTTAGTT |
| | | CAGATCCAAACTACACTATAGGGTGACTTATTGTCGATCAAAATTATTATATGTAA |
| | | GTAATAGATCATACATCAACACCGAGGTCTTTGTCCAATAGAAATAGTATGTCCTG |
| | | GAGTTTTATCAAATACCTGCCATGTGCAAGTTCACAGAATAGGACGCTTCTACAGA |
| | | ATTCATAAAATCCCACATCCTTAGCGTAAGTTGTCAGATGAATTAATTATATTTTT |
| | | GATACGGCCCCAGTTATTCTCGAAGTCCACTCTTAAAAAAAGTTATTGTACGAACT |
| | | TGCATAAATCGATAACCTGTTACCAACATGCCCCGGCATAAATCAACAACGTGGTT |
| | | CGGATACGACAATATCAATCAATCCGAAATTCAAAATAGAATATTCAACTTGACTT |
| | | AATCGCAGTTCATTCGTGAATAGACACATATTAGCTCTCGCGCGCTTTCTTATCTT |
| | | CACAGCTTCTTCTCGATACCTGAATAAGTACGGGACCATTTATGTTCATAAGCATT |
| | | CAGTGAAACTGCAGTCTAAATACTATTGGCATATACTTAT |
| 96 | 40.20% | GATATGCCATCTATCGAGGCCTGTTAGCTTAGGACATTACATGACAGTGAGACCTA |
| | | GATATATAGTTGCATGAGTAGATGTAACCGAAGGTACTCAGGGACAGAACTGACGG |
| | | ATTGACGTTTTTCAGTATCGTAAAAGTTTGAGATCCAACAATGAAAGCTTGATGCG |
| | | CCAGATGATGGAAATGCGCAAACTGTCGTGTGATAACACGGGAATTGGTGCTAAGC |
| | | TGGAATGGTCTAATTCAAGTTCCAATCCATATCCATCTATGTGCGAGGAATTTGTA |
| | | ACGGTAATTATATTGCCTTACAATTATTATCAACCAACACACTTGAACGATGTAAT |
| | | TGGGGGTATATACCAATAATAGTACTGCCAACTACTGTTTTTTGCAAGAATTAATC |
| | | GTAGTCCGAATTAAAAGAAAAGACGGTGTACGCAACCCAAGTAATTAAACGAATAA |
| | | TCATACGGTCGATATGCTCATTCGATAAAACGCGAGATCTTTAAGTTCTCTCACCG |
| | | GGGTAATGCATAATTGCCTTAATTGGAAATTGCTTTAGGTGAGAGTCAGTAAACCA |
| | | TTGGTGAGATGTGGTTATACTGCACCTCACGCAAATTAATATTCTAACTTTAACCT |
| | | GAATTATGGGTTCCCCTCATCGGGAAGTATATCTAGTGCCAACCTATCACAGTTGC |
| | | GCACATATGTTTAGAAATGGTTAGTCGGTCAGGGGAACTCACGTAAGCGGTAGTAG |
| | | TAGAATTTAATTTATGGTCTCCTAAAGCATCGACATAGTACACTGCGACCATTCTA |
| | | ACACATACTAAACTTTGAACTTACTGATATCTTTTATGTTTGACTTCCTTGCTACG |
| | | CAAGTCCAGGCCCAGACAGCTGAGTTGTCCTTACACGAGCTATTTGCTGATCATAT |
| | | GGTTTAATCGGCACGCGAATTGCAAGTTTGATTTAAGGTGAGCGCATACTTGAATA |
| | | CAGCCAGGGAGCTCCCTACTCAGCGATCGTCTTCAGAGATTTCACGAAAATATAAG |
| | | CATTCCCATCAGAAATTCTAATTAAACCTTACCGGAGGTGGGGATTACTCGCAGAG |
| | | TTAAATAATGAGCCCACATTATGCGTTTGCTTCTGGAGATTATGGGTGGTTTTTCC |
| | | CGTACCGCCTAATATAGTATGCTTCGACTCAGCAACTTCACTCTAAACCCTAGAGA |
| | | GCCTCTGTATGTACGCGCGTGGATGAAATCAAGAATGGTTGGAGTCAATGACTGGG |
| | | GCACAAGTGTAATCTGGTTCGATTAATACATGGCACTAGGTGCTACGAGGACGAGT |
| | | GAATGCAATATATGAGTCCTTGCTAATAAGCATCGAAGATACTCTCCGGTACTCCT |
| | | TCATATTCGACTAATCGGTGCACTCAACTTTAGGGGGGCTCCTTATTATAAAATAC |
| | | ATATAGGGTTTGTTTAAATGATTTGTTCTATTAATACGGGCAAAATTAATGCAATG |
| | | TTCACCTAGGCACGTTGGTACTCGCCGCCAAACATTGGCATTAATGGGGATACTTA |
| | | GAAACAACATAACATGAAAAATATCTAGGAACGCCAACATATACGCCGTGACCGTC |
| | | TGTCTTAATAGACTCTTTTTGTTTAAAGGGTACTGAGTGATTAACTAATGCTTTCC |
| | | AATCCTTTCCGTTAGAAGGCTATTACTACAAGTGTTTCCCACGTGCCGTTAAAAAT |
| | | AGAATTATCTTTGTGGGTTTACGAGCGCGTACTGAAAACAGGTTTCTTGGATGGGA |
| | | TAATATTATAGATAGCAATAAAGTAAACTGGAAAACAGTATTGGATAGCATGTGAT |
| | | GGACCTTGACCCCCTTGTGGCATAAGATAATCTCAGCGTTTCGTTACACTTACATT |
| | | CACTGTTAATGTCTATAGGCAAGTTACTATTTGGAGTATTTCAAAGTGAACGGAAG |
| | | AAATAGAAGTGCTAACAAACTCCGTCATAGTAGGATCATATCTCCAGAGCGACCTC |
| | | ATACATGCTAAAAACCTAGTAGACTTCGTACTATGGATTT |
| 97 | 40.60% | AAGACACTTTACCACATAAGTAAACCGTTGACATTATCGTGGCGGAGAGATACTGC |
| | | TTGTACTGGGACACTCAGTATTTTGTGGAATATTGTACCTAGCGCCTCGTTCCGTG |
| | | AAAGTGTGGCATGGATTTTCATAATTTTATGCTGTCCTCATTGCCTACAATTAATC |
| | | CAGTAAGCACTAGAGAAATATCTGCTCCTATGCTGAGATTAGCCTTATGAGGTCTT |
| | | TATATCTTTCTGTAAAGGCCATTGTTCTTTTGATCCTGGAGTCTCTGAATTTTGAT |
| | | TTGTCCCTCAAAGCCTTATGTGTACCCGGTCCCGGAGCATGAAGACGTATATCTTG |
| | | AAGTAATCCGAAAGTATTTAGGTGTCGTTGTCCAGTAGTAATCCCGGTTATGGGTT |
| | | ATAATTAAGTGTTAACATCCGAGCTTGGTCTGTATAATAGTGTGTTTGAATAGTAA |
| | | ATATCAGGACTCTACAGGGACCTATTCTACTTCGGGTTGTGTATCTTCCTTGGAAT |
| | | AACTTTTGCTACGCAAAAAAGCTATAACAAGGTCTGGAGACGGATGTGATTTAGTA |
| | | GGGCAAATAGATTTAGGTCTTCGATAGTACAGAATACTATGCTACAACCAATCTCT |
| | | TCATGGCTTTATCAATACAATGTTCTTCCTTAACTCAGACGGGAGCAATTATAGTT |
| | | AGCTGAAGGTTGCCTCACAATATGTGTCAGAGCTAGCGAAAAGCTCCTACCAATAT |
| | | ACATCAGATAAGGAGTTCATACATCTGTGGCCGATCAAGCAAGCAAGGCCGTCCGG |
| | | TTCACGACCTGGGTAGTCTGAGTTTGGAGGAGAAGCCATCGCCTCTCGCATTCTAC |
| | | TAGAGAAAGATTTCACACTTACTGACAGAGCTACACTGGTACGACGAATCTACAAA |
| | | ACTAAGCAAAGTCCTAGGGTGAGCAATGCATGGTAACTAGTACGATTGATCAGTGC |
| | | GTGGTATACTATCCGGATAGTCCAGACGTCAAGACCTAATCATCGTACGTAATTAA |
| | | ATAATAATGCATTCAACTCTTCGGATACGATATATACTTATATGCATTAACTATAC |
| | | TTTCTCATGCATTGTATCTAACAAAATCTGTACGGCAGAATTAATTACTAAAGTCT |
| | | TAATGATTCGAATATTAATATCAATTTTATTACGAAACAACCAAACTGACAACGTA |
| | | GAGAGGCAACTACCCAGAGTCGCCAAGAATACTGTTTACGAATTGTAGAAAAGATG |
| | | TAAGAATGTTCGGATGTCGGATTACTTAATTGCGAACGTTTGTCAAGTCGTTGCAG |
| | | GATACCCTCATCTCCTCTTCCTAGTGAATTATCTGAAAGTACTATTATACAATCTA |
| | | AATCGGATACATTCGTTTGTAACACCACATGGTTGGCTCAGCTGACCATTTACGCG |
| | | CGATATTCTGTGCTATCCGAAGGCGTAAAAGGAATTCAAGTCAGTCTCCTCTTCGT |
| | | TATGTAGAAAGGGAGGACTCCTCCGCCGTATATTCAGCTGGCTTTAACTAGGAACA |
| | | TAGTTGCAGTTCAAACAGTAGAAAATCCTGGAAGACATTTCTTGATAGTCTATCTC |
| | | AGAAAAAGGGGGGTGACGTTCATGTTTACTAAGACTTGAAATGTGGCTCCGTATCT |
| | | GCACAACCAGGTTTGGGCGGATGCCGGCCGCCATGTAACACTGAACCTCGCAAGAA |
| | | ATGCACAATTGAACAAATGAATACTCACATCTTATCGCTTAATGTTAAATTCAAGG |
| | | CGAGACTGGCTCGAATTATTGGAGCCTATGAAGATGTATATTAATGCCAAGGCACC |
| | | GCACATAGTAAAGACTATACTAACCAAGTGTGATATTCAATCGATCGTTGTGGGGA |
| | | ATCAGGTACAGTTAGTGGCGAACAGCTTTGACATCCGTTTAACTTTGGCAGCACCA |
| | | CAAACCCTTTGCGTACGTTTTTGTGTTATAACCAAGTTATGTTGCAACCTACTTTG |
| | | ACCTCTTATTTCTTTGCCGCAAGACTGAATGTCGTATTAT |
| 98 | 41.50% | GAGCAACCTACGGATATACTATCGATTCTGGACATGGTAAGTGTGTTGCGTGGTTA |
| | | ATAAAAAGATTTCGTGGTCGGGGGTAGATATACCTGTAAGGTTTCCAACAGACCGC |
| | | TTTGTAGAAAGAGACTTAGTCCCTTTGCAAAATGAGGGGACCGACTAAGAAAGCGT |
| | | TGAATTCAGGTAATACTTTTTGACGTTACCATAGTTGTTGCAGTCCCGGAGTTAAA |
| | | CAGAGACACATCGTGGCGGAGTCCGTAGTATCGCATGCGTGGATTTATTGTTGTAA |
| | | TCAGATGTTCAATATGGCGTCAATATACAAATAAACAGGTCAGATGGAGTTAGCCT |
| | | TACTTAAAAAACGAAAACAATGTATGCCCTAAGCAAAAAAACTAGATAAGGACGAT |
| | | CACCACAGTTTTAAGAGATCTATATGCCCCTTTGACATCCTTATTCTGACAATGGG |
| | | CAGATCCAACTACAAGATGTCGTACCGCTAACACTTGACTAACTAACGTCAAGTAA |
| | | AAAGTTCGTTAGTCATATTATCAAGTATGGACTTATTCATCGACAGGTTGTAATTA |
| | | GCCCTCCCCTAGATTAGCTGGGCTGAACCCCTATTCCTACGCTCCCTTGTCACATG |
| | | TATTCTCTACCTCAATAGGCCGGAAACTCGCAAGCCCAAGTATAGCGTACGGATTA |
| | | AATTCGCGCAATCGCTCTTGACCATGTTAAATGCTTGCGCGTAACATCGAAAAGGA |
| | | GGCAAGACATTTCAGAAGTAACATATCAGTTGACGGCTTACGGTGCTGAGGTTTAA |
| | | AATCCGACTGATTGCTATCCTATCGCTGAGGAATGACTAACCTTGCAAATCCAAGT |
| | | CTAGAACTGTCCTAGTTCTGTACCATGCCCAGCGTTCGGATGTCAGTACGTGTATG |
| | | CAGCATTTAGGAGGTGATGTCTCCCAGTCGGTCAATAAGCTTTGCTTACCTCACGG |
| | | ATAACTAAGTTCATCTCCAGTGTACGAAGATTCTCTAGCACTAACTATTCATTGTA |
| | | ACTAATTGGTATCCGACTTTAAGCCATAGTGTGGCATGACGTAAGTTATGTCAGTT |
| | | CTTTGGAACTTTTTGCGCAGCTGTGTTGACGAAACACAGGTTGCAGGTTGGTCTAG |
| | | GTAAGGGATGCACTCACTGCGATGTGATCCTTTAATGGCCATTTAAATCTATCTCG |
| | | AGTATAGCGTGTATACTTACTATGAAGCAAATTAGTATACATATAACAATGAATAT |
| | | ACACATAGTGGGAGGTTGCCATTCATCCATGTAGGCATGTAATATGGCACCTCCTC |
| | | TTTGGATACAGAGGCCCATGCCTCCGAATCACATATTTACTTAAACAGTTAACGGA |
| | | ATTCAGGTATCCCGTTTCATTATTCGAAACGTCTCTGGGGTTACCTTACTTACGTT |
| | | ATCTGCATGAGAATAGAGTCCATCGGCGTTTCTAACAATCAATCATGCTTGCAATT |
| | | CAGCGAGTGTAGAGGAATTGTAAGAACGCCGGATGCTCCCTTTACCTTATCCGCAC |
| | | AGGCCCCTACGATTGAACTATTGAAAGTTTTATTACAAATCTCATATATGGGGGAG |
| | | CAGTTAAAGTTCTGCATAAGAAGGACCTAGGATAATGCCATAAAAGGTTGATATGG |
| | | AAATACTATTGGAATAAGAAAGTATATGGTGTCTATAATGGATATATCAGTAAACG |
| | | AAGGCATTTCTTACACTTTGATTTCATTAACTGTAATCTCTATTTGTGTTGGCGAA |
| | | TCCGGTAAACAGAGGTTTATAACTGGTTTACCTTAGTCGAGTGTCTTAGATATACA |
| | | TGTCGATTCAGATCAATCCTACTCATCCCAAACGCACATGTCACGATACGTACTTT |
| | | ATACAGTAAGAGGCACAATGTGGGTGCCCTCTCTCGTCCGACTTATTGCGGACGGA |
| | | GAAATAGTTAGTACGGACTGTCACAAGTCTGTAACCACTAAAGATCGGGCAGCTCA |
| | | GACATTATTGAAGGTAGGCCAAAGTATCATTAATGCTTTG |
| 99 | 39.90% | ATTAATAAATGTCTAACGGTCTAGAAATGCACCTAATTTGCTACTGCTGAACTCCT |
| | | GATTACTCCTCCTCGTTTATACTTGTTCATTAAGAATTTTTTCCGTCTAGATTAAG |
| | | TACACGGTAATACACACGATTAAATACACCGCCACAGATCTTCGCTATCAATATTA |
| | | CATTTTGTTCACTCATTACGATAAGCGTGGCTTGGCTGAGTTCTAGACTTATCGTG |
| | | TTAACGTCAATGAAAACTTATGGATTTGAAGCTACGATGCTAATCTAACTTTACCT |
| | | TAAGCAAGAAAGACCTTCGTTAATAGGACCCTTAAAGCCTGTGATGTCGGTTAAAC |
| | | GGTTCTAGTTTGATAGTGACGTTAGGGACTCGGTATACATCTTAGCCGAACTGTCT |
| | | AAATTACTTTAGAGAAACTTTTCCCTGGGGGAGGCACGTTCCGTTTATGGACCTCA |
| | | TTTGAGACTCAATATGTACAACTAATAGTGTGATTAGATCCTGATTCCCATACGTA |
| | | TCGGCTCGCCCTTAATCAATACAGATCCGTGCTATGTCCATACTGCGATTCCAAAG |
| | | GTTGTCTAACAAGACAAACTTGAGAGAGGCTTCACAAAGCAACCCAGCACCCTTGT |
| | | CCTCTTTTTTAGGGGTACGCTGACATCTGGATGCATTAAGAAATACGTATCTAGAA |
| | | GGATCGCGATAAGTCGCACAAGTTTACCACCTTATATTCTGCAGGCTGCTATTGGA |
| | | GGTAATACGTGCTCGCACACGCCCAAGTGAGGCATTCTTACAAGACTTACCTTACA |
| | | GCCTATTAATAACGTCGAATTTTGCGCAGCAACCAATTCCAGGGCAAACTATAAGC |
| | | CTTATTGAGGTTAATAGGGCGCAATATATTTACGATAGAAGGTAAATCTATAATAC |
| | | TGTCACTTGTCAATGATGATGGTCTAACTAATTGATTCCCATGCAAGTGGCGAACC |
| | | AGGCTTACTTTAGTTTAATAGCGATCAAGTATACTAAGCACACACTGAATGTATCA |
| | | CATAAGATACGTAAAATAAATCAACTCATTAAATCAAAGACAGATTCACAAATGTT |
| | | TCGTGTTTTAACAGATCTGAATATAAACTCTGCTGATGTGATCGTAGGACGTAAGA |
| | | AGGTATAGTTGAAGAATAGCGTGAATATCTGATCTCTGTTAGCAAATACATCACGA |
| | | TTATCACCAGGTTTACCACAACAATAAGATTGTGACTGACACTACTTTCTATATGA |
| | | ATGTATTCTCATGAGGATGCGTAAGACGTATAGGATCATACTGAATTATAACTCCA |
| | | TATTAGGGTCTATATCACATACATCTCCAAGTTAAAAAGTCTATTGGCGATTCCAC |
| | | ACAACTCGCGCTAGTAGTACATTTTACCGGTACCGGTACAGTCTAAGTTATTGATC |
| | | TAGGTTCAACTTCTAAAATACTGAAGTCTCAGGTATATAGAATTTATACTACTCGC |
| | | GGGACGTAAAGCCCCTCTGTGGTTAGCGTCGCAGCGTCGAGTAAATTCCTTATAGA |
| | | GCCTAAACCTTGATAATTTCGACGTACCGTTATAACGCAATTAATAGACTTCTCAT |
| | | TTTCCTGCCGAGTCGGGTCTGGTATAGTCTAGGACGGGGGTAGATATGATCGTCGT |
| | | CTTCTCTAATCTAATTTAATCTATAACCACAGCGTACAAGTAAGGTATGTAAGATA |
| | | CAGAGATAAATTAGAGATTTGTGTTACTCCGCATGTTGAACTAAACCCAAAGGTTC |
| | | ACGCCGTATGCCTTTCAAGTTCCTCCGCTCAAAAGGCTCCGGGTGTCCCCTACCCG |
| | | ATATGGCGGAAATCGTTAATTCTCATAACGACCAACCTTACCTTGGACACACCTAA |
| | | GCACTAAGTCGGTAAATGGAGTACACAATGTGGGAGTTGTGTTTAACATAATGAGG |
| | | CTCGTTCAGACTATGTTCGAGGCGTATAACGATTTGTGACAGATTCCTCATCAACT |
| | | CGGGTCAGATTTATAGCAATGGTAAATTCCCTATATCCTA |
| 100 | 39.60% | TATGGTGTGGCACATATGAATAAAACAAGGAGAAGCAGCCGACAATACTTAGAACG |
| | | TGTCAGAACAATCAAGATGTCTGAAACGTTCAACAATCGAGTTATTCCGGGCTAAT |
| | | TTATTCCCATCCTTATATACAGAGCCGCACAATACCAAGTAACGTGCTTTGGGCCA |
| | | CGAACTCACTCTAGTCTTCCGGACCCTCCGGTACTACTCGGTATGGTGGATATTCA |
| | | TGAGAATGGTTTTAGTCTTAAAAAAATGTGAACAAGAAAACATTTACGTCCAAGAA |
| | | AGCGGTATTTTGTTTGGGTCTAGGAAACAATCAGTCGTGGACCTGGGCGAGATCGG |
| | | CTGTTTTCGACCGATTTTATGCTAAGCAGAAGGAAGTGACCGAGGTTGTGTTTAGA |
| | | TCCAGTAAAAGTCGTCATACCCGAGGAGATTTCTGTGGTGCCTAGTGACTAGCGAT |
| | | CCCGTGCAGCAGTTCAAATGCGCTGGATAGTTCGCTCCTGCACCACTAGTTCACAC |
| | | CAGAAGTATGTCTTTTAAGAGACTGTCTAAGAAATATAGTCTCTAAACGTGACTAT |
| | | CGTTCACTCCCTGTACAAATCTAGGACTAACGGGTATAGATTAAACGTATTAGAAT |
| | | TTCGGAGCATTAGAATTTTGTTGTTCTAAGTTAGGATGATTTCAAGTGTCCATGTA |
| | | AATTGAGGTCAATATAGGACGATCTACATCCGAGATAGGCCAAGTACGATTCTGTG |
| | | TTACATTTTGCGTTCGCACAAGCTAGGACGAGGGTATGAGCATTTTGTGCTAACCG |
| | | AATGAGATGCAGCTTATTGTATCCTTACCCGCAACATAGGGCATGAAGGCGTGGTT |
| | | CGAGAATCGCGCGAGATAAATACATGTTTCGATTTATGTCAACCACTGCAATGGTT |
| | | TATAAATGTTATTCAAGCATCGATTCAATAACCTCTGGATGTAGTAATATCTGCGG |
| | | GTGTGTAAGTGCGATATCCTAAGTCGGGAGATTTAACAATACCTTGGGATGCTCCG |
| | | GACAATTTTCGACGTACGCAATTATGAACATGCATTGATTGACTAAACTTAAGAAA |
| | | CATAATCAGTGTATAGTATTGTAACAATGGATTCTGAGTGTCTAATGTTTTCTCGC |
| | | TCCATGTTATAACACATAATTATACTTATAATACCATCCCATCTTTAAGTACAAAA |
| | | CCTTGTTGCGCTGCTTTATGGAGACTATTGAGCCCAACGGGTTGAGTGGTTATTAC |
| | | TATTTGAAGTAAAAGCAGTATCTACTCAGATTCCTAGAGGTAAATATGAACTTGTT |
| | | TTCTATCTGGTTATCTATTTTTAGTTTTATGGATATGGACGAAGTTAAAAGTTATA |
| | | GACCTGACATTCTTCTCCCATAGGTATAGAAGTGGAGTTAAACAAGTTCTTAGTGG |
| | | GGGAAATGACGTACAGACTACTATCTTGATGATAGCTTTTCGATCAAACAAGAGTT |
| | | TCAACCGCTGTAAAGGTTTATATGCGATGTAGTGTGGTACGATAACGTACTTTGCC |
| | | GATCATTCACTGATTCCATTAGGTACGACACTCTCAGTTACAAAGCGGTACTAACC |
| | | TAGCAAAAAGTGAATATCGCCCTACAAACTATTACTGGAGTGCGGTGGCAGCTTTG |
| | | GCGAAAATTGGCCGAACTCTTTGCTGTTTATATGGTAACTATTCTCACTATGCTAC |
| | | TGATTGGAAAAAGATATTTGCCAACTAATAGTCGTAATGTTAGTATTGATAGGGAT |
| | | AATAGGCATTTAAAGTTCCCTGAAACATACGGTAAATAAGATCTCTTTTAACAACA |
| | | CCAGGGGTGGCTCACTGGGGTAGCAAATACTTAACGATCCCTTTTTCATCAAGTGA |
| | | GTTATCTGCTTTGGATTCTTACAACTAGATGTTATAAAGAAAGAAGCTGCGCAGTT |
| | | TGCATGACTAAAATTTATATGAAGTAGTAGTTATTAGTACTATCTCTTAGTAGGCT |
| | | AGAATGTAAACCTGCAGACATCATGGAATGCACATACCCG |
| 101 | 38.40% | TCAATAGCCCAGTCGGTTTTGTTAGATACATTTTATCGAATCTGTAAAGATATTTT |
| | | ATAATAAGATAATATCAGCGCCTAGCTGCGGAATTCCACTCAGAGAATACCTCTCC |
| | | TGAATATCAGCCTTAGTGGCGTTATACGATATTTCACACTCTCAAAATCCCGAGTC |
| | | AGACTATACCCGCGCATGTTTAGTAAAGGTTGATTCTGAGATCTCGAGTCCAAAAA |
| | | AGATACCCACTACTTTAAAGATTTGCATTCAGTTGTTCCATCGGCCTGGGTAGTAA |
| | | AGGGGGTATGCTCGCTCCGAGTCGATGGAACTGTAAATGTTAGCCCTGATACGCGG |
| | | AACATATCAGTAACAATCTTTACCTAATATGGAGTGGGATTAAGCTTCATAGAGGA |
| | | TATGAAACGCTCGTAGTATGGCTTCCTACATAAGTAGAATTATTAGCAACTAAGAT |
| | | ATTACCACTGCCCAATAAAAGAGATTCCACTTAGATTCATAGGTAGTCCCAACAAT |
| | | CATGTCTGAATACTAAATTGATCAATTGGACTATGTCAAAATTATTTTGAAGAAGT |
| | | AATCATCAACTTAGGCGCTTTTTAGTGTTAAGAGCGCGTTATTGCCAACCGGGCTA |
| | | AACCTGTGTAACTCTTCAATATTGTATATAATTATAGGCAGAATAAGCTATGAGTG |
| | | CATTATGAGATAAACATAGATTTTTGTCCACTCGAAATATTTGAATTTCTTGATCC |
| | | TGGGCTAGTTCAGCCATAAGTTTTCACTAATAGTTAGGACTACCAATTACACTACA |
| | | TTCAGTTGCTGAAATTCACATCACTGCCGCAATATTTATGAAGCTATTATTGCATT |
| | | AAGACTTAGGAGATAAATACGAAGTTGATATATTTTTCAGAATCAGCGAAAAGACC |
| | | CCCTATTGACATTACGAATTCGAGTTTAACGAGCACATAAATCAAACACTACGAGG |
| | | TTACCAAGATTGTATCTTACATTAATGCTATCCAGCCAGCCGTCATGTTTAACTGG |
| | | ATAGTCATAATTAATATCCAATGATCGTTTCACGTAGCTGCATATCGAGGAAGTTG |
| | | TATAATTGAAAACCCACACATTAGAATGCATGGTGCATCGCTAGGGTTTATCTTAT |
| | | CTTGCTCGTGCCAAGAGTGTAGAAAGCCACATATTGATACGGAAGCTGCCTAGGAG |
| | | GTTGGTATATGTTGATTGTGCTCACCATCTCCCTTCCTAATCTCCTAGTGTTAAGT |
| | | CCAATCAGTGGGCTGGCTCTGGTTAAAAGTAATATACACGCTAGATCTCTCTACTA |
| | | TAATACAGGCTAAGCCTACGCGCTTTCAATGCACTGATTACCAACTTAGCTACGGC |
| | | CAGCCCCATTTAATGAATTATCTCAGATGAATTCAGACATTATTCTCTACAAGGAC |
| | | ACTTTAGAGTGTCCTGCGGAGGCATAATTATTATCTAAGATGGGGTAAGTCCGATG |
| | | GAAGACACAGATACATCGGACTATTCCTATTAGCCGAGAGTCAACCGTTAGAACTC |
| | | GGAAAAAGACATCGAAGCCGGTAACCTACGCACTATAAATTTCCGCAGAGACATAT |
| | | GTAAAGTTTTATTAGAACTGGTATCTTGATTACGATTCTTAACTCTCATACGCCGG |
| | | TCCGGAATTTGTGACTCGAGAAAATGTAATGACATGCTCCAATTGATTTCAAAATT |
| | | AGATTTAAGGTCAGCGAACTATGTTTATTCAACCGTTTACAACGCTATTATGCGCG |
| | | ATGGATGGGGCCTTGTATCTAGAAACCGAATAATAACATACCTGTTAAATGGCAAA |
| | | CTTAGATTATTGCGATTAATTCTCACTTCAGAGGGTTATCGTGCCGAATTCCTGAC |
| | | TTTGGAATAATAAAGTTGATATTGAGGTGCAATATCAACTACACTGGTTTAACCTT |
| | | TAAACACATGGAGTCAAGTTTTCGCTATGCCAGCCGGTTATGCAGCTAGGATTAAT |
| | | ATTAGAGCTCTTTTCTAATTCGTCCTAATAATCTCTTCAC |

In one embodiment, the first stuffer has a sequence comprising at least 5, at least 10, at least 15, at least 20, at least 25, at least 30, at least 35, at least 40, at least 45, at least 50, at least 55, at least 60, at least 65, at least 70, at least 75, at least 80, at least 85, at least 90, at least 95, at least 100, at least 105, at least 110, at least 115, at least 120, at least 125, at least 130, at least 135, at least 140, at least 145, at least 150, at least 155, at least 160, at least 165, at least 170, at least 175, at least 180, at least 185, at least 190, at least 195, at least 200, at least 205, at least 210, at least 215, at least 220, at least 225, at least 230, at least 235, at least 240, at least 245, at least 250, at least 275, at least 300, at least 325, at least 350, at least 375, at least 400, at least 425, at least 450, at least 475, or at least 500 nucleotides of a sequence set forth in Table 2. In another embodiment, the second stuffer has a sequence comprising at least 5, at least 10, at least 15, at least 20, at least 25, at least 30, at least 35, at least 40, at least 45, at least 50, at least 55, at least 60, at least 65, at least 70, at least 75, at least 80, at least 85, at least 90, at least 95, at least 100, at least 105, at least 110, at least 115, at least 120, at least 125, at least 130, at least 135, at least 140, at least 145, at least 150, at least 155, at least 160, at least 165, at least 170, at least 175, at least 180, at least 185, at least 190, at least 195, at least 200, at least 205, at least 210, at least 215, at least 220, at least 225, at least 230, at least 235, at least 240, at least 245, at least 250, at least 275, at least 300, at least 325, at least 350, at least 375, at least 400, at least 425, at least 450, at least 475, or at least 500 nucleotides of a sequence set forth in Table 2.

It is preferable that the stuffer sequence not interfere with the resolution of the cleavage site at the target nucleic acid. Thus, the stuffer sequence should have minimal sequence identity to the nucleic acid sequence at the cleavage site of the target nucleic acid. In some embodiments, the stuffer sequence is less than 80%, 70%, 60%, 55%, 50%, 45%, 40%, 35%, 30%, 25%, 20%, or 10% identical to any nucleic acid sequence within 500, 450, 400, 350, 300, 250, 200, 150, 100, 50 nucleotides from the cleavage site of the target nucleic acid. In some embodiments, the stuffer sequence is less than 80%, 70%, 60%, 55%, 50%, 45%, 40%, 35%, 30%, 25%, 20%, or 10% identical to any nucleic acid sequence within 500, 450, 400, 350, 300, 250, 200, 150, 100, 50 base pairs from the cleavage site of the target nucleic acid.

In order to avoid off-target molecular recombination events, it is preferable that the stuffer sequence have minimal homology to a nucleic acid sequence in the genome of the target cell. In some embodiments, the stuffer sequence has minimal sequence identity to a nucleic acid in the genome of the target cell. In some embodiments, the stuffer sequence is less than 80%, 70%, 60%, 55%, 50%, 45%, 40%, 35%, 30%, 25%, 20%, or 10% identical to any nucleic acid sequence of the same length (as measured in base pairs or nucleotides) in the genome of the target cell. In some embodiments, a 20 base pair stretch of the stuffer sequence is less than 80%, 70%, 60%, 55%, 50%, 45%, 40%, 35%, 30%, 25%, 20%, or 10% identical to any at least 20 base pair stretch of nucleic acid of the target cell genome. In some embodiments, a 20 nucleotide stretch of the stuffer sequence is less than 60%, 55%, 50%, 45%, 40%, 35%, 30%, 25%, 20%, or 10% identical to any at least 20 nucleotide stretch of nucleic acid of the target cell genome.

In some embodiments, the stuffer sequence has minimal sequence identity to a nucleic acid sequence in the donor template (e.g., the nucleic acid sequence of the cargo, or the nucleic acid sequence of a priming site present in the donor template). In some embodiments, the stuffer sequence is less than 80%, 70%, 60%, 55%, 50%, 45%, 40%, 35%, 30%, 25%, 20%, or 10% identical to any nucleic acid sequence of the same length (as measured in base pairs or nucleotides) in the donor template. In some embodiments, a 20 base pair stretch of the stuffer sequence is less than 80%, 70%, 60%, 55%, 50%, 45%, 40%, 35%, 30%, 25%, 20%, or 10% identical to any 20 base pair stretch of nucleic acid of the donor template. In some embodiments, a 20 nucleotide stretch of the stuffer sequence is less than 80%, 70%, 60%, 55%, 50%, 45%, 40%, 35%, 30%, 25%, 20%, or 10% identical to any 20 nucleotide stretch of nucleic acid of the donor template.

In some embodiments, the length of the first homology arm and its adjacent stuffer sequence (i.e., A1+S1) is approximately equal to the length of the second homology arm and its adjacent stuffer sequence (i.e., A2+S2). For example, in some embodiments the length of A1+S1 is the same as the length of A2+S2 (as determined in base pairs or nucleotides). In some embodiments, the length of A1+S1 differs from the length of A2+S2 by 25 nucleotides or less. In some embodiments, the length of A1+S1 differs from the length of A2+S2 by 24, 23, 22, 21, 20, 19 18, 17, 16, 15, 14, 13, 12, 11, 10, 9, 8, 7, 6, 5, 4, 3, or 2 nucleotides or less. In some embodiments, the length of A1+S1 differs from the length of A2+S2 by 25 base pairs or less. In some embodiments, the length of A1+S1 differs from the length of A2+S2 by 24, 23, 22, 21, 20, 19 18, 17, 16, 15, 14, 13, 12, 11, 10, 9, 8, 7, 6, 5, 4, 3, or 2 base pairs or less.

In some embodiments, the length of A1+H1 is 250 base pairs or less. In some embodiments, the length of A1+H1 is 200 base pairs or less. In some embodiments, the length of A1+H1 is 150 base pairs or less. In some embodiments, the length of A1+H1 is 100 base pairs or less. In some embodiments, the length of A1+H1 is 50 base pairs or less. In some embodiments, the length of A1+H1 is 250, 200, 190, 180, 170, 160, 150, 140, 130, 120, 110, 100, 90, 80, 70, 60, 50, 49, 48, 47, 46, 45, 44, 43, 42, 41, 40, 39, 38, 37, 36, 35, 34, 33, 32, 31, 30, 29, 28, 27, 26, 25, 24, 23, 22, 21, or 20 base pairs. In some embodiments, the length of A1+H1 is 40 base pairs. In some embodiments, the length of A2+H2 is 250 base pairs or less. In some embodiments, the length of A2+H2 is 200 base pairs or less. In some embodiments, the length of A2+H2 is 150 base pairs or less. In some embodiments, the length of A2+H2 is 100 base pairs or less. In some embodiments, the length of A2+H2 is 50 base pairs or less. In some embodiments, the length of A2+H2 is 250, 200, 190, 180, 170, 160, 150, 140, 130, 120, 110, 100, 90, 80, 70, 60, 50, 49, 48, 47, 46, 45, 44, 43, 42, 41, 40, 39, 38, 37, 36, 35, 34, 33, 32, 31, 30, 29, 28, 27, 26, 25, 24, 23, 22, 21, or 20 base pairs. In some embodiments, the length of A2+H2 is 40 base pairs.

In some embodiments, the length of A1+S1 is the same as the length of H1+X+H2 (as determined in nucleotides or base pairs). In some embodiments, the length of A1+S1 differs from the length of H1+X+H2 by less than 25 nucleotides. In some embodiments, the length of A1+S1 differs from the length of H1+X+H2 by 24, 23, 22, 21, 20, 19 18, 17, 16, 15, 14, 13, 12, 11, 10, 9, 8, 7, 6, 5, 4, 3, or 2 nucleotides. In some embodiments, the length of A1+S1 differs from the length of H1+X+H2 by less than 25 base pairs. In some embodiments, the length of A1+S1 differs from the length of H1+X+H2 by 24, 23, 22, 21, 20, 19 18, 17, 16, 15, 14, 13, 12, 11, 10, 9, 8, 7, 6, 5, 4, 3, or 2 base pairs.

In some embodiments, the length of A2+S2 is the same as the length of H1+X+H2 (as determined in nucleotides or base pairs). In some embodiments, the length of A2+S2 differs from the length of H1+X+H2 by less than 25 nucleotides. In some embodiments, the length of A2+S2 differs from the length of H1+X+H2 by 24, 23, 22, 21, 20, 19 18, 17, 16, 15, 14, 13, 12, 11, 10, 9, 8, 7, 6, 5, 4, 3, or 2 nucleotides. In some embodiments, the length of A2+S2 differs from the length of H1+X+H2 by less than 25 base pairs. In some embodiments, the length of A2+S2 differs from the length of H1+X+H2 by 24, 23, 22, 21, 20, 19 18, 17, 16, 15, 14, 13, 12, 11, 10, 9, 8, 7, 6, 5, 4, 3, or 2 base pairs.

### Genome Editing Systems

The term “genome editing system” refers to any system having RNA-guided DNA editing activity. In addition to a donor template, described above, genome editing systems of the present disclosure include at least two components adapted from naturally occurring CRISPR systems: a guide RNA (gRNA) and an RNA-guided nuclease. These two components form a complex that is capable of associating with a specific nucleic acid sequence and editing the DNA in or around that nucleic acid sequence, for instance by making one or more of a single-strand break (an SSB or nick), a double-strand break (a DSB) and/or a point mutation. In certain embodiments, the genome editing system is a transiently active genome editing system. In certain embodiments, the genome editing system can alter both a cellular endogenous target gene and the RNA-guided-nuclease expression. In certain embodiments, the gRNA/RNA-guided nuclease complex can cleave both the nucleic acid encoding the RNA-guided nuclease and the nucleic acid encoding the cellular endogenous target gene.

Naturally occurring CRISPR systems are organized evolutionarily into two classes and five types (Makarova et al. Nat Rev Microbiol. 2011 June; 9(6): 467-477 (Makarova), incorporated by reference herein), and while genome editing systems of the present disclosure may adapt components of any type or class of naturally occurring CRISPR system, the embodiments presented herein are generally adapted from Class 2, and type II or V CRISPR systems. Class 2 systems, which encompass types II and V, are characterized by relatively large, multidomain RNA-guided nuclease proteins (e.g., Cas9 or Cpf1) and one or more guide RNAs (e.g., a crRNA and, optionally, a tracrRNA) that form ribonucleoprotein (RNP) complexes that associate with (i.e. target) and cleave specific loci complementary to a targeting (or spacer) sequence of the crRNA. Genome editing systems according to the present disclosure similarly target and edit cellular DNA sequences, but differ significantly from CRISPR systems occurring in nature. For example, the unimolecular guide RNAs described herein do not occur in nature, and both guide RNAs and RNA-guided nucleases according to this disclosure may incorporate any number of non-naturally occurring modifications.

Genome editing systems can be implemented (e.g., administered or delivered to a cell or a subject) in a variety of ways, and different implementations may be suitable for distinct applications. For instance, a genome editing system is implemented, in certain embodiments, as a protein/RNA complex (a ribonucleoprotein, or RNP), which can be included in a pharmaceutical composition that optionally includes a pharmaceutically acceptable carrier and/or an encapsulating agent, such as a lipid or polymer micro- or nano-particle, micelle, liposome, etc. In certain embodiments, a genome editing system is implemented as one or more nucleic acids encoding the RNA-guided nuclease and guide RNA components described above (optionally with one or more additional components); in certain embodiments, the genome editing system is implemented as one or more vectors comprising such nucleic acids, for instance a viral vector such as an adeno-associated virus; and in certain embodiments, the genome editing system is implemented as a combination of any of the foregoing. Additional or modified implementations that operate according to the principles set forth herein will be apparent to the skilled artisan and are within the scope of this disclosure.

It should be noted that the genome editing systems of the present disclosure can be targeted to a single specific nucleotide sequence, or may be targeted to—and capable of editing in parallel—two or more specific nucleotide sequences through the use of two or more guide RNAs. The use of multiple gRNAs is referred to as “multiplexing” throughout this disclosure, and can be employed to target multiple, unrelated target sequences of interest, or to form multiple SSBs or DSBs within a single target domain and, in some cases, to generate specific edits within such target domain. For example, International Patent Publication No. WO 2015/138510 by Maeder et al. (Maeder), which is incorporated by reference herein, describes a genome editing system for correcting a point mutation (C.2991+1655A to G) in the human CEP290 gene that results in the creation of a cryptic splice site, which in turn reduces or eliminates the function of the gene. The genome editing system of Maeder utilizes two guide RNAs targeted to sequences on either side of (i.e. flanking) the point mutation, and forms DSBs that flank the mutation. This, in turn, promotes deletion of the intervening sequence, including the mutation, thereby eliminating the cryptic splice site and restoring normal gene function.

As another example, WO 2016/073990 by Cotta-Ramusino, et al. (“Cotta-Ramusino”), incorporated by reference herein, describes a genome editing system that utilizes two gRNAs in combination with a Cas9 nickase (a Cas9 that makes a single strand nick such as S. pyogenes D10A), an arrangement termed a “dual-nickase system.” The dual-nickase system of Cotta-Ramusino is configured to make two nicks on opposite strands of a sequence of interest that are offset by one or more nucleotides, which nicks combine to create a double strand break having an overhang (5′ in the case of Cotta-Ramusino, though 3′ overhangs are also possible). The overhang, in turn, can facilitate homology directed repair events in some circumstances. And, as another example, WO 2015/070083 by Palestrant et al. (“Palestrant”, incorporated by reference herein) describes a gRNA targeted to a nucleotide sequence encoding Cas9 (referred to as a “governing RNA”), which can be included in a genome editing system comprising one or more additional gRNAs to permit transient expression of a Cas9 that might otherwise be constitutively expressed, for example in some virally transduced cells. These multiplexing applications are intended to be exemplary, rather than limiting, and the skilled artisan will appreciate that other applications of multiplexing are generally compatible with the genome editing systems described here.

Genome editing systems can, in some instances, form double strand breaks that are repaired by cellular DNA double-strand break mechanisms such as NHEJ or HDR. These mechanisms are described throughout the literature, for example by Davis & Maizels, PNAS, 111(10): E924-932, Mar. 11, 2014 (Davis) (describing Alt-HDR); Frit et al. DNA Repair 17(2014) 81-97 (Frit) (describing Alt-NHEJ); and Iyama and Wilson III, DNA Repair (Amst.) 2013-August; 12(8): 620-636 (Iyama) (describing canonical HDR and NHEJ pathways generally).

Where genome editing systems operate by forming DSBs, such systems optionally include one or more components that promote or facilitate a particular mode of double-strand break repair or a particular repair outcome. For instance, Cotta-Ramusino also describes genome editing systems in which a single-stranded oligonucleotide “donor template” is added; the donor template is incorporated into a target region of cellular DNA that is cleaved by the genome editing system, and can result in a change in the target sequence.

In certain embodiments, genome editing systems modify a target sequence, or modify expression of a gene in or near the target sequence, without causing single- or double-strand breaks. For example, a genome editing system may include an RNA-guided nuclease fused to a functional domain that acts on DNA, thereby modifying the target sequence or its expression. As one example, an RNA-guided nuclease can be connected to (e.g., fused to) a cytidine deaminase functional domain, and may operate by generating targeted C-to-A substitutions. Exemplary nuclease/deaminase fusions are described in Komor et al. Nature 533, 420-424 (19 May 2016) (“Komor”), which is incorporated by reference. Alternatively, a genome editing system may utilize a cleavage-inactivated (i.e. a “dead”) nuclease, such as a dead Cas9 (dCas9), and may operate by forming stable complexes on one or more targeted regions of cellular DNA, thereby interfering with functions involving the targeted region(s) including, without limitation, mRNA transcription, chromatin remodeling, etc.

Guide RNA (gRNA) Molecules

The terms “guide RNA” and “gRNA” refer to any nucleic acid that promotes the specific association (or “targeting”) of an RNA-guided nuclease such as a Cas9 or a Cpf1 to a target sequence such as a genomic or episomal sequence in a cell. gRNAs can be unimolecular (comprising a single RNA molecule, and referred to alternatively as chimeric), or modular (comprising more than one, and typically two, separate RNA molecules, such as a crRNA and a tracrRNA, which are usually associated with one another, for instance by duplexing). gRNAs and their component parts are described throughout the literature, for instance in Briner et al. (Molecular Cell 56(2), 333-339, Oct. 23, 2014 (Briner), which is incorporated by reference), and in Cotta-Ramusino.

In bacteria and archaea, type II CRISPR systems generally comprise an RNA-guided nuclease protein such as Cas9, a CRISPR RNA (crRNA) that includes a 5′ region that is complementary to a foreign sequence, and a trans-activating crRNA (tracrRNA) that includes a 5′ region that is complementary to, and forms a duplex with, a 3′ region of the crRNA. While not intending to be bound by any theory, it is thought that this duplex facilitates the formation of—and is necessary for the activity of—the Cas9/gRNA complex. As type II CRISPR systems were adapted for use in gene editing, it was discovered that the crRNA and tracrRNA could be joined into a single unimolecular or chimeric guide RNA, for instance, but not by way of limitation, by means of a four nucleotide (e.g., GAAA) “tetraloop” or “linker” sequence bridging complementary regions of the crRNA (at its 3′ end) and the tracrRNA (at its 5′ end). (Mali et al. Science. 2013 Feb. 15; 339(6121): 823-826 (“Mali”); Jiang et al. Nat Biotechnol. 2013 March; 31(3): 233-239 (“Jiang”); and Jinek et al., 2012 Science August 17; 337(6096): 816-821 (“Jinek”), all of which are incorporated by reference herein.)

Guide RNAs, whether unimolecular or modular, include a “targeting domain” that is fully or partially complementary to a target domain within a target sequence, such as a DNA sequence in the genome of a cell where editing is desired. Targeting domains are referred to by various names in the literature, including without limitation “guide sequences” (Hsu et al., Nat Biotechnol. 2013 September; 31(9): 827-832. (“Hsu”), incorporated by reference herein), “complementarity regions” (Cotta-Ramusino), “spacers” (Briner) and generically as “crRNAs” (Jiang). Irrespective of the names they are given, targeting domains are typically 10-30 nucleotides in length, and in certain embodiments are 16-24 nucleotides in length (for instance, 16, 17, 18, 19, 20, 21, 22, 23 or 24 nucleotides in length), and are at or near the 5′ terminus of in the case of a Cas9 gRNA, and at or near the 3′ terminus in the case of a Cpf1 gRNA.

In addition to the targeting domains, gRNAs typically (but not necessarily, as discussed below) include a plurality of domains that may influence the formation or activity of gRNA/Cas9 complexes. For instance, as mentioned above, the duplexed structure formed by first and secondary complementarity domains of a gRNA (also referred to as a repeat:anti-repeat duplex) interacts with the recognition (REC) lobe of Cas9 and can mediate the formation of Cas9/gRNA complexes. (Nishimasu et al., Cell 156, 935-949, Feb. 27, 2014 (Nishimasu 2014) and Nishimasu et al.. Cell 162, 1113-1126, Aug. 27, 2015 (Nishimasu 2015), both incorporated by reference herein). It should be noted that the first and/or second complementarity domains may contain one or more poly-A tracts, which can be recognized by RNA polymerases as a termination signal. The sequence of the first and second complementarity domains are, therefore, optionally modified to eliminate these tracts and promote the complete in vitro transcription of gRNAs, for instance through the use of A-G swaps as described in Briner, or A-U swaps. These and other similar modifications to the first and second complementarity domains are within the scope of the present disclosure.

Along with the first and second complementarity domains, Cas9 gRNAs typically include two or more additional duplexed regions that are involved in nuclease activity in vivo but not necessarily in vitro. (Nishimasu 2015). A first stem-loop one near the 3′ portion of the second complementarity domain is referred to variously as the “proximal domain,” (Cotta-Ramusino) “stem loop 1” (Nishimasu 2014 and 2015) and the “nexus” (Briner). One or more additional stem loop structures are generally present near the 3′ end of the gRNA, with the number varying by species: S. pyogenes gRNAs typically include two 3′ stem loops (for a total of four stem loop structures including the repeat:anti-repeat duplex), while S. aureus and other species have only one (for a total of three stem loop structures). A description of conserved stem loop structures (and gRNA structures more generally) organized by species is provided in Briner.

While the foregoing description has focused on gRNAs for use with Cas9, it should be appreciated that other RNA-guided nucleases have been (or may in the future be) discovered or invented which utilize gRNAs that differ in some ways from those described to this point. For instance, Cpf1 (“CRISPR from Prevotella and franciscella 1”) is a recently discovered RNA-guided nuclease that does not require a tracrRNA to function. (Zetsche et al., 2015, Cell 163, 759-771 Oct. 22, 2015 (Zetsche I), incorporated by reference herein). A gRNA for use in a Cpf1 genome editing system generally includes a targeting domain and a complementarity domain (alternately referred to as a “handle”). It should also be noted that, in gRNAs for use with Cpf1, the targeting domain is usually present at or near the 3′ end, rather than the 5′ end as described above in connection with Cas9 gRNAs (the handle is at or near the 5′ end of a Cpf1 gRNA).

Those of skill in the art will appreciate, however, that although structural differences may exist between gRNAs from different prokaryotic species, or between Cpf1 and Cas9 gRNAs, the principles by which gRNAs operate are generally consistent. Because of this consistency of operation, gRNAs can be defined, in broad terms, by their targeting domain sequences, and skilled artisans will appreciate that a given targeting domain sequence can be incorporated in any suitable gRNA, including a unimolecular or modular gRNA, or a gRNA that includes one or more chemical modifications and/or sequential modifications (substitutions, additional nucleotides, truncations, etc.). Thus, for economy of presentation in this disclosure, gRNAs may be described solely in terms of their targeting domain sequences.

More generally, skilled artisans will appreciate that some aspects of the present disclosure relate to systems, methods and compositions that can be implemented using multiple RNA-guided nucleases. For this reason, unless otherwise specified, the term gRNA should be understood to encompass any suitable gRNA that can be used with any RNA-guided nuclease, and not only those gRNAs that are compatible with a particular species of Cas9 or Cpf1. By way of illustration, the term gRNA can, in certain embodiments, include a gRNA for use with any RNA-guided nuclease occurring in a Class 2 CRISPR system, such as a type II or type V or CRISPR system, or an RNA-guided nuclease derived or adapted therefrom.

gRNA Design

Methods for selection and validation of target sequences as well as off-target analyses have been described previously, e.g., in Mali; Hsu; Fu et al., 2014 Nat. Biotechnol. 32(3): 279-84, Heigwer et al., 2014 Nat Methods 11(2):122-3; Bae et al. (2014) Bioinformatics 30(10): 1473-5; and Xiao A et al. (2014) Bioinformatics 30(8): 1180-1182. Each of these references is incorporated by reference herein. As a non-limiting example, gRNA design can involve the use of a software tool to optimize the choice of potential target sequences corresponding to a user's target sequence, e.g., to minimize total off-target activity across the genome. While off-target activity is not limited to cleavage, the cleavage efficiency at each off-target sequence can be predicted, e.g., using an experimentally-derived weighting scheme. These and other guide selection methods are described in detail in Maeder and Cotta-Ramusino.

gRNA Modifications

The activity, stability, or other characteristics of gRNAs can be altered through the incorporation of certain modifications. As one example, transiently expressed or delivered nucleic acids can be prone to degradation by, e.g., cellular nucleases. Accordingly, the gRNAs described herein can contain one or more modified nucleosides or nucleotides which introduce stability toward nucleases. While not wishing to be bound by theory it is also believed that certain modified gRNAs described herein can exhibit a reduced innate immune response when introduced into cells. Those of skill in the art will be aware of certain cellular responses commonly observed in cells, e.g., mammalian cells, in response to exogenous nucleic acids, particularly those of viral or bacterial origin. Such responses, which can include induction of cytokine expression and release and cell death, may be reduced or eliminated altogether by the modifications presented herein.

Certain exemplary modifications discussed in this section can be included at any position within a gRNA sequence including, without limitation at or near the 5′ end (e.g., within 1-10, 1-5, or 1-2 nucleotides of the 5′ end) and/or at or near the 3′ end (e.g., within 1-10, 1-5, or 1-2 nucleotides of the 3′ end). In some cases, modifications are positioned within functional motifs, such as the repeat-anti-repeat duplex of a Cas9 gRNA, a stem loop structure of a Cas9 or Cpf1 gRNA, and/or a targeting domain of a gRNA.

As one example, the 5′ end of a gRNA can include a eukaryotic mRNA cap structure or cap analog (e.g., a G(5′)ppp(5′)G cap analog, a m7G(5′)ppp(5′)G cap analog, or a 3′-O-Me-m7G(5′)ppp(5′)G anti reverse cap analog (ARCA)), as shown below:

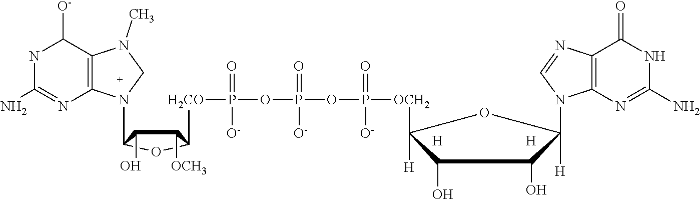

The cap or cap analog can be included during either chemical synthesis or in vitro transcription of the gRNA.

Along similar lines, the 5′ end of the gRNA can lack a 5′ triphosphate group. For instance, in vitro transcribed gRNAs can be phosphatase-treated (e.g., using calf intestinal alkaline phosphatase) to remove a 5′ triphosphate group.

Another common modification involves the addition, at the 3′ end of a gRNA, of a plurality (e.g., 1-10, 10-20, or 25-200) of adenine (A) residues referred to as a polyA tract. The polyA tract can be added to a gRNA during chemical synthesis, following in vitro transcription using a polyadenosine polymerase (e.g., E. coli Poly(A)Polymerase), or in vivo by means of a polyadenylation sequence, as described in Maeder.

It should be noted that the modifications described herein can be combined in any suitable manner, e.g., a gRNA, whether transcribed in vivo from a DNA vector, or in vitro transcribed gRNA, can include either or both of a 5′ cap structure or cap analog and a 3′ polyA tract.

Guide RNAs can be modified at a 3′ terminal U ribose. For example, the two terminal hydroxyl groups of the U ribose can be oxidized to aldehyde groups and a concomitant opening of the ribose ring to afford a modified nucleoside as shown below:

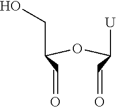

wherein “U” can be an unmodified or modified uridine.

The 3′ terminal U ribose can be modified with a 2′3′ cyclic phosphate as shown below:

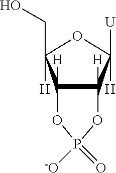

wherein “U” can be an unmodified or modified uridine.

Guide RNAs can contain 3′ nucleotides which can be stabilized against degradation, e.g., by incorporating one or more of the modified nucleotides described herein. In certain embodiments, uridines can be replaced with modified uridines, e.g., 5-(2-amino)propyl uridine, and 5-bromo uridine, or with any of the modified uridines described herein; adenosines and guanosines can be replaced with modified adenosines and guanosines, e.g., with modifications at the 8-position, e.g., 8-bromo guanosine, or with any of the modified adenosines or guanosines described herein.

In certain embodiments, sugar-modified ribonucleotides can be incorporated into the gRNA, e.g., wherein the 2′ OH-group is replaced by a group selected from H, —OR, —R (wherein R can be, e.g., alkyl, cycloalkyl, aryl, aralkyl, heteroaryl or sugar), halo, —SH, —SR (wherein R can be, e.g., alkyl, cycloalkyl, aryl, aralkyl, heteroaryl or sugar), amino (wherein amino can be, e.g., NH₂; alkylamino, dialkylamino, heterocyclyl, arylamino, diarylamino, heteroarylamino, diheteroarylamino, or amino acid); or cyano (—CN). In certain embodiments, the phosphate backbone can be modified as described herein, e.g., with a phosphothioate (PhTx) group. In certain embodiments, one or more of the nucleotides of the gRNA can each independently be a modified or unmodified nucleotide including, but not limited to 2′-sugar modified, such as, 2′-O-methyl, 2′-O-methoxyethyl, or 2′-Fluoro modified including, e.g., 2′-F or 2′-O-methyl, adenosine (A), 2′-F or 2′-O-methyl, cytidine (C), 2′-F or 2′-O-methyl, uridine (U), 2′-F or 2′-O-methyl, thymidine (T), 2′-F or 2′-O-methyl, guanosine (G), 2′-O-methoxyethyl-5-methyluridine (Teo), 2′-O-methoxyethyladenosine (Aeo), 2′-O-methoxyethyl-5-methylcytidine (m5Ceo), and any combinations thereof.

Guide RNAs can also include “locked” nucleic acids (LNA) in which the 2′ OH-group can be connected, e.g., by a C1-6 alkylene or C1-6 heteroalkylene bridge, to the 4′ carbon of the same ribose sugar. Any suitable moiety can be used to provide such bridges, include without limitation methylene, propylene, ether, or amino bridges; O-amino (wherein amino can be, e.g., NH₂; alkylamino, dialkylamino, heterocyclyl, arylamino, diarylamino, heteroarylamino, or diheteroarylamino, ethylenediamine, or polyamino) and aminoalkoxy or O(CH₂)ₙ-amino (wherein amino can be, e.g., NH₂; alkylamino, dialkylamino, heterocyclyl, arylamino, diarylamino, heteroarylamino, or diheteroarylamino, ethylenediamine, or polyamino).

In certain embodiments, a gRNA can include a modified nucleotide which is multicyclic (e.g., tricyclo; and “unlocked” forms, such as glycol nucleic acid (GNA) (e.g., R-GNA or S-GNA, where ribose is replaced by glycol units attached to phosphodiester bonds), or threose nucleic acid (TNA, where ribose is replaced with α-L-threofuranosyl-(3′→2′)).

Generally, gRNAs include the sugar group ribose, which is a 5-membered ring having an oxygen. Exemplary modified gRNAs can include, without limitation, replacement of the oxygen in ribose (e.g., with sulfur (S), selenium (Se), or alkylene, such as, e.g., methylene or ethylene); addition of a double bond (e.g., to replace ribose with cyclopentenyl or cyclohexenyl); ring contraction of ribose (e.g., to form a 4-membered ring of cyclobutane or oxetane); ring expansion of ribose (e.g., to form a 6- or 7-membered ring having an additional carbon or heteroatom, such as for example, anhydrohexitol, altritol, mannitol, cyclohexanyl, cyclohexenyl, and morpholino that also has a phosphoramidate backbone). Although the majority of sugar analog alterations are localized to the 2′ position, other sites are amenable to modification, including the 4′ position. In certain embodiments, a gRNA comprises a 4′-S, 4′-Se or a 4′-C-aminomethyl-2′-O-Me modification.

In certain embodiments, deaza nucleotides, e.g., 7-deaza-adenosine, can be incorporated into the gRNA. In certain embodiments, O- and N-alkylated nucleotides, e.g., N6-methyl adenosine, can be incorporated into the gRNA. In certain embodiments, one or more or all of the nucleotides in a gRNA are deoxynucleotides.

### RNA-Guided Nucleases

RNA-guided nucleases according to the present disclosure include, but are not limited to, naturally-occurring Class 2 CRISPR nucleases such as Cas9, and Cpf1, as well as other nucleases derived or obtained therefrom. In functional terms, RNA-guided nucleases are defined as those nucleases that: (a) interact with (e.g., complex with) a gRNA; and (b) together with the gRNA, associate with, and optionally cleave or modify, a target region of a DNA that includes (i) a sequence complementary to the targeting domain of the gRNA and, optionally, (ii) an additional sequence referred to as a “protospacer adjacent motif,” or “PAM,” which is described in greater detail below. As the following examples will illustrate, RNA-guided nucleases can be defined, in broad terms, by their PAM specificity and cleavage activity, even though variations may exist between individual RNA-guided nucleases that share the same PAM specificity or cleavage activity. Skilled artisans will appreciate that some aspects of the present disclosure relate to systems, methods and compositions that can be implemented using any suitable RNA-guided nuclease having a certain PAM specificity and/or cleavage activity. For this reason, unless otherwise specified, the term RNA-guided nuclease should be understood as a generic term, and not limited to any particular type (e.g., Cas9 vs. Cpf1), species (e.g., S. pyogenes vs. S. aureus) or variation (e.g., full-length vs. truncated or split; naturally-occurring PAM specificity vs. engineered PAM specificity, etc.) of RNA-guided nuclease.

The PAM sequence takes its name from its sequential relationship to the “protospacer” sequence that is complementary to gRNA targeting domains (or “spacers”). Together with protospacer sequences, PAM sequences define target regions or sequences for specific RNA-guided nuclease/gRNA combinations.

Various RNA-guided nucleases may require different sequential relationships between PAMs and protospacers. In general, Cas9s recognize PAM sequences that are 3′ of the protospacer as visualized relative to the top or complementary strand:
5′-------------------[protospacer]-----------------------------3′
3′-----------------------------------[PAM]-------------------5′

Cpf1, on the other hand, generally recognizes PAM sequences that are 5′ of the protospacer:
5′-----------------------------[protospacer]------------------3′
3′--------------------[PAM]-----------------------------------5′

In addition to recognizing specific sequential orientations of PAMs and protospacers, RNA-guided nucleases can also recognize specific PAM sequences. S. aureus Cas9, for instance, recognizes a PAM sequence of NNGRRT or NNGRRV, wherein the N residues are immediately 3′ of the region recognized by the gRNA targeting domain. S. pyogenes Cas9 recognizes NGG PAM sequences. And F. novicida Cpf1 recognizes a TTN PAM sequence. PAM sequences have been identified for a variety of RNA-guided nucleases, and a strategy for identifying novel PAM sequences has been described by Shmakov et al., 2015, Molecular Cell 60, 385-397, Nov. 5, 2015. It should also be noted that engineered RNA-guided nucleases can have PAM specificities that differ from the PAM specificities of reference molecules (for instance, in the case of an engineered RNA-guided nuclease, the reference molecule may be the naturally occurring variant from which the RNA-guided nuclease is derived, or the naturally occurring variant having the greatest amino acid sequence homology to the engineered RNA-guided nuclease).

In addition to their PAM specificity, RNA-guided nucleases can be characterized by their DNA cleavage activity: naturally-occurring RNA-guided nucleases typically form DSBs in target nucleic acids, but engineered variants have been produced that generate only SSBs (discussed above) Ran & Hsu, et al., Cell 154(6), 1380-1389, Sep. 12, 2013 (Ran), incorporated by reference herein), or that do not cut at all.

Cas9

Crystal structures have been determined for S. pyogenes Cas9 (Jinek 2014), and for S. aureus Cas9 in complex with a unimolecular guide RNA and a target DNA (Nishimasu 2014; Anders 2014; and Nishimasu 2015).

A naturally occurring Cas9 protein comprises two lobes: a recognition (REC) lobe and a nuclease (NUC) lobe; each of which comprise particular structural and/or functional domains. The REC lobe comprises an arginine-rich bridge helix (BH) domain, and at least one REC domain (e.g., a REC1 domain and, optionally, a REC2 domain). The REC lobe does not share structural similarity with other known proteins, indicating that it is a unique functional domain. While not wishing to be bound by any theory, mutational analyses suggest specific functional roles for the BH and REC domains: the BH domain appears to play a role in gRNA:DNA recognition, while the REC domain is thought to interact with the repeat:anti-repeat duplex of the gRNA and to mediate the formation of the Cas9/gRNA complex.

The NUC lobe comprises a RuvC domain, an HNH domain, and a PAM-interacting (PI) domain. The RuvC domain shares structural similarity to retroviral integrase superfamily members and cleaves the non-complementary (i.e. bottom) strand of the target nucleic acid. It may be formed from two or more split RuvC motifs (such as RuvC I, RuvC II, and RuvC III in S. pyogenes and S. aureus). The HNH domain, meanwhile, is structurally similar to HNN endonuclease motifs, and cleaves the complementary (i.e. top) strand of the target nucleic acid. The PI domain, as its name suggests, contributes to PAM specificity.

While certain functions of Cas9 are linked to (but not necessarily fully determined by) the specific domains set forth above, these and other functions may be mediated or influenced by other Cas9 domains, or by multiple domains on either lobe. For instance, in S. pyogenes Cas9, as described in Nishimasu 2014, the repeat: antirepeat duplex of the gRNA falls into a groove between the REC and NUC lobes, and nucleotides in the duplex interact with amino acids in the BH, PI, and REC domains. Some nucleotides in the first stem loop structure also interact with amino acids in multiple domains (PI, BH and REC1), as do some nucleotides in the second and third stem loops (RuvC and PI domains).

Cpf1

The crystal structure of Acidaminococcus sp. Cpf1 in complex with crRNA and a double-stranded (ds) DNA target including a TTTN PAM sequence has been solved by Yamano et al. (Cell. 2016 May 5; 165(4): 949-962 (Yamano), incorporated by reference herein). Cpf1, like Cas9, has two lobes: a REC (recognition) lobe, and a NUC (nuclease) lobe. The REC lobe includes REC1 and REC2 domains, which lack similarity to any known protein structures. The NUC lobe, meanwhile, includes three RuvC domains (RuvC-I, -II and -III) and a BH domain. However, in contrast to Cas9, the Cpf1 REC lobe lacks an HNH domain, and includes other domains that also lack similarity to known protein structures: a structurally unique PI domain, three Wedge (WED) domains (WED-I, -II and -III), and a nuclease (Nuc) domain.

While Cas9 and Cpf1 share similarities in structure and function, it should be appreciated that certain Cpf1 activities are mediated by structural domains that are not analogous to any Cas9 domains. For instance, cleavage of the complementary strand of the target DNA appears to be mediated by the Nuc domain, which differs sequentially and spatially from the HNH domain of Cas9. Additionally, the non-targeting portion of Cpf1 gRNA (the handle) adopts a pseudoknot structure, rather than a stem loop structure formed by the repeat:antirepeat duplex in Cas9 gRNAs.

Modifications of RNA-Guided Nucleases

The RNA-guided nucleases described above have activities and properties that can be useful in a variety of applications, but the skilled artisan will appreciate that RNA-guided nucleases can also be modified in certain instances, to alter cleavage activity, PAM specificity, or other structural or functional features.

Turning first to modifications that alter cleavage activity, mutations that reduce or eliminate the activity of domains within the NUC lobe have been described above. Exemplary mutations that may be made in the RuvC domains, in the Cas9 HNH domain, or in the Cpf1 Nuc domain are described in Ran and Yamano, as well as in Cotta-Ramusino. In general, mutations that reduce or eliminate activity in one of the two nuclease domains result in RNA-guided nucleases with nickase activity, but it should be noted that the type of nickase activity varies depending on which domain is inactivated. As one example, inactivation of a RuvC domain of a Cas9 will result in a nickase that cleaves the complementary or top strand as shown below (where C denotes the site of cleavage):
5′-------------------[protospacer]--[C]---------------------3′
3′--------------------------------------------------------------5′

On the other hand, inactivation of a Cas9 HNH domain results in a nickase that cleaves the bottom or non-complementary strand:
5′-------------------[protospacer]---------------------------3′
3′-------------------------------------[C]---------------------5′

Modifications of PAM specificity relative to naturally occurring Cas9 reference molecules has been described by Kleinstiver et al. for both S. pyogenes (Kleinstiver et al., Nature. 2015 Jul. 23; 523(7561):481-5 (Kleinstiver I) and S. aureus (Kleinstiver et al., Nat Biotechnol. 2015 December; 33(12): 1293-1298 (Klienstiver II)). Kleinstiver et al. have also described modifications that improve the targeting fidelity of Cas9 (Nature, 2016 Jan. 28; 529, 490-495 (Kleinstiver III)). Each of these references is incorporated by reference herein.

RNA-guided nucleases have been split into two or more parts, as described by Zetsche et al. (Nat Biotechnol. 2015 February; 33(2):139-42 (Zetsche II), incorporated by reference), and by Fine et al. (Sci Rep. 2015 Jul. 1; 5:10777 (Fine), incorporated by reference).

RNA-guided nucleases can be, in certain embodiments, size-optimized or truncated, for instance via one or more deletions that reduce the size of the nuclease while still retaining gRNA association, target and PAM recognition, and cleavage activities. In certain embodiments, RNA guided nucleases are bound, covalently or non-covalently, to another polypeptide, nucleotide, or other structure, optionally by means of a linker. Exemplary bound nucleases and linkers are described by Guilinger et al., Nature Biotechnology 32, 577-582 (2014), which is incorporated by reference for all purposes herein.

RNA-guided nucleases also optionally include a tag, such as, but not limited to, a nuclear localization signal to facilitate movement of RNA-guided nuclease protein into the nucleus. In certain embodiments, the RNA-guided nuclease can incorporate C- and/or N-terminal nuclear localization signals. Nuclear localization sequences are known in the art and are described in Maeder and elsewhere.

The foregoing list of modifications is intended to be exemplary in nature, and the skilled artisan will appreciate, in view of the instant disclosure, that other modifications may be possible or desirable in certain applications. For brevity, therefore, exemplary systems, methods and compositions of the present disclosure are presented with reference to particular RNA-guided nucleases, but it should be understood that the RNA-guided nucleases used may be modified in ways that do not alter their operating principles. Such modifications are within the scope of the present disclosure.

Zinc Finger Nucleases (ZFNs) and Transcription Activator-Like Effector Nucleases (TALENs)

In addition to the CRISPR nucleases described above, there are several widely used systems to introduce targeted cuts into the genomes of cells, including Zinc Finger Nucleases (ZFNs) Transcription Activator-Like Effector Nucleases (TALENs), and Argonaute nuclease systems. See, e.g., Gaj et al., Trends in Biotechnology 31(7): 397-405 (2013); Gao et al., Nature Biotechnology, 34, 768-733 (2016); and Carlson et al., Molecular Therapy Nucleic Acids 1(1):e3 (2012). Accordingly, the methods and systems described herein can employ ZFNs and/or TALENs to cleave a target nucleic acid at a cut site. Once cleaved, the donor templates described herein may be integrated into the target nucleic acid at the cleavage site.

Accordingly, in some embodiments, the invention provides a method of altering a cell, comprising forming, in a target nucleic acid of the cell, a break, (e.g., a single-strand break or a double-strand break) at a cleavage site using a ZFN or TALEN, wherein the target nucleic acid comprises a first strand comprising wherein the target nucleic acid comprises a first strand comprising: a first homology arm 5′ to the cleavage site, a first priming site either within the first homology arm or 5′ to the first homology arm, a second homology arm 3′ to the cleavage site, and a second priming site either within the second homology arm or 3′ to the second homology arm, and recombining an exogenous oligonucleotide donor template with the target nucleic acid by homologous recombination to produce an altered nucleic acid, wherein a first strand of the exogenous oligonucleotide donor template comprises either: i) a cargo, a priming site that is substantially identical to the second priming site either within or 5′ to the cargo, a first donor homology arm 5′ to the cargo, and a second donor homology arm 3′ to the cargo; or ii) a cargo, a first donor homology arm 5′ to the cargo, a priming site that is substantially identical to the first priming site either within or 3′ to the cargo, and a second donor homology arm 3′ to the cargo, thereby altering the cell. Additional aspects of the invention set forth herein may likewise employ ZFNs and/or TALENs in place of CRISPR nucleases to generate a single- or double-stranded break at a cleavage site in a target nucleic acid.

### Nucleic Acids Encoding RNA-Guided Nucleases

Nucleic acids encoding RNA-guided nucleases, e.g., Cas9, Cpf1 or functional fragments thereof, are provided herein. Exemplary nucleic acids encoding RNA-guided nucleases have been described previously (see, e.g., Cong 2013; Wang 2013; Mali 2013; Jinek 2012).

In some cases, a nucleic acid encoding an RNA-guided nuclease can be a synthetic nucleic acid sequence. For example, the synthetic nucleic acid molecule can be chemically modified. In certain embodiments, an mRNA encoding an RNA-guided nuclease will have one or more (e.g., all) of the following properties: it can be capped; polyadenylated; and substituted with 5-methylcytidine and/or pseudouridine.

Synthetic nucleic acid sequences can also be codon optimized, e.g., at least one non-common codon or less-common codon has been replaced by a common codon. For example, the synthetic nucleic acid can direct the synthesis of an optimized messenger mRNA, e.g., optimized for expression in a mammalian expression system, e.g., described herein. Examples of codon optimized Cas9 coding sequences are presented in Cotta-Ramusino.

In addition, or alternatively, a nucleic acid encoding an RNA-guided nuclease may comprise a nuclear localization sequence (NLS). Nuclear localization sequences are known in the art.

### Functional Analysis of Candidate Molecules

Candidate RNA-guided nucleases, gRNAs, and complexes thereof, can be evaluated by standard methods known in the art. See, e.g., Cotta-Ramusino. The stability of RNP complexes may be evaluated by differential scanning fluorimetry, as described below.

Differential Scanning Fluorimetry (DSF)

The thermostability of ribonucleoprotein (RNP) complexes comprising gRNAs and RNA-guided nucleases can be measured via DSF. The DSF technique measures the thermostability of a protein, which can increase under favorable conditions such as the addition of a binding RNA molecule, e.g., a gRNA.

A DSF assay can be performed according to any suitable protocol, and can be employed in any suitable setting, including without limitation (a) testing different conditions (e.g., different stoichiometric ratios of gRNA: RNA-guided nuclease protein, different buffer solutions, etc.) to identify optimal conditions for RNP formation; and (b) testing modifications (e.g., chemical modifications, alterations of sequence, etc.) of an RNA-guided nuclease and/or a gRNA to identify those modifications that improve RNP formation or stability. One readout of a DSF assay is a shift in melting temperature of the RNP complex; a relatively high shift suggests that the RNP complex is more stable (and may thus have greater activity or more favorable kinetics of formation, kinetics of degradation, or another functional characteristic) relative to a reference RNP complex characterized by a lower shift. When the DSF assay is deployed as a screening tool, a threshold melting temperature shift may be specified, so that the output is one or more RNPs having a melting temperature shift at or above the threshold. For instance, the threshold can be 5-10° C. (e.g., 5°, 6°, 7°, 8°, 9°, 10°) or more, and the output may be one or more RNPs characterized by a melting temperature shift greater than or equal to the threshold.

Two non-limiting examples of DSF assay conditions are set forth below:

To determine the best solution to form RNP complexes, a fixed concentration (e.g., 2 μM) of Cas9 in water+10×SYPRO Orange® (Life Technologies cat #S-6650) is dispensed into a 384 well plate. An equimolar amount of gRNA diluted in solutions with varied pH and salt is then added. After incubating at room temperature for 10′ and brief centrifugation to remove any bubbles, a Bio-Rad CFX384™ Real-Time System C1000 Touch™ Thermal Cycler with the Bio-Rad CFX Manager software is used to run a gradient from 20° ° C. to 90° ° C. with a 1° C. increase in temperature every 10 seconds.

The second assay consists of mixing various concentrations of gRNA with fixed concentration (e.g., 2 μM) Cas9 in optimal buffer from assay 1 above and incubating (e.g., at RT for 10′) in a 384 well plate. An equal volume of optimal buffer+10×SYPRO Orange® (Life Technologies cat #S-6650) is added and the plate sealed with Microseal® B adhesive (MSB-1001). Following brief centrifugation to remove any bubbles, a Bio-Rad CFX384™ Real-Time System C1000 Touch™ Thermal Cycler with the Bio-Rad CFX Manager software is used to run a gradient from 20° ° C. to 90° ° C. with a 1° C. increase in temperature every 10 seconds.

### Genome Editing Strategies

The genome editing systems described above are used, in various embodiments of the present disclosure, to generate edits in (i.e. to alter) targeted regions of DNA within or obtained from a cell. Various strategies are described herein to generate particular edits, and these strategies are generally described in terms of the desired repair outcome, the number and positioning of individual edits (e.g., SSBs or DSBs), and the target sites of such edits.

Genome editing strategies that involve the formation of SSBs or DSBs are characterized by repair outcomes including: (a) deletion of all or part of a targeted region; (b) insertion into or replacement of all or part of a targeted region; or (c) interruption of all or part of a targeted region. This grouping is not intended to be limiting, or to be binding to any particular theory or model, and is offered solely for economy of presentation. Skilled artisans will appreciate that the listed outcomes are not mutually exclusive and that some repairs may result in other outcomes. The description of a particular editing strategy or method should not be understood to require a particular repair outcome unless otherwise specified.

Replacement of a targeted region generally involves the replacement of all or part of the existing sequence within the targeted region with a homologous sequence, for instance through gene correction or gene conversion, two repair outcomes that are mediated by HDR pathways. HDR is promoted by the use of a donor template, which can be single-stranded or double-stranded, as described in greater detail below. Single- or double-stranded templates can be exogenous, in which case they will promote gene correction, or they can be endogenous (e.g., a homologous sequence within the cellular genome), to promote gene conversion. Exogenous templates can have asymmetric overhangs (i.e. the portion of the template that is complementary to the site of the DSB may be offset in a 3′ or 5′ direction, rather than being centered within the donor template), for instance as described by Richardson et al. (Nature Biotechnology 34, 339-344 (2016), (Richardson), incorporated by reference). In instances where the template is single-stranded, it can correspond to either the complementary (top) or non-complementary (bottom) strand of the targeted region.

Gene conversion and gene correction are facilitated, in some cases, by the formation of one or more nicks in or around the targeted region, as described in Ran and Cotta-Ramusino. In some cases, a dual-nickase strategy is used to form two offset SSBs that, in turn, form a single DSB having an overhang (e.g., a 5′ overhang).

Interruption and/or deletion of all or part of a targeted sequence can be achieved by a variety of repair outcomes. As one example, a sequence can be deleted by simultaneously generating two or more DSBs that flank a targeted region, which is then excised when the DSBs are repaired, as is described in Maeder for the LCA10 mutation. As another example, a sequence can be interrupted by a deletion generated by formation of a double strand break with single-stranded overhangs, followed by exonucleolytic processing of the overhangs prior to repair.

One specific subset of target sequence interruptions is mediated by the formation of an indel within the targeted sequence, where the repair outcome is typically mediated by NHEJ pathways (including Alt-NHEJ). NHEJ is referred to as an “error prone” repair pathway because of its association with indel mutations. In some cases, however, a DSB is repaired by NHEJ without alteration of the sequence around it (a so-called “perfect” or “scarless” repair); this generally requires the two ends of the DSB to be perfectly ligated. Indels, meanwhile, are thought to arise from enzymatic processing of free DNA ends before they are ligated that adds and/or removes nucleotides from either or both strands of either or both free ends.

Because the enzymatic processing of free DSB ends may be stochastic in nature, indel mutations tend to be variable, occurring along a distribution, and can be influenced by a variety of factors, including the specific target site, the cell type used, the genome editing strategy used, etc. It is possible to draw limited generalizations about indel formation: deletions formed by repair of a single DSB are most commonly in the 1-50 bp range, but can reach greater than 100-200 bp. Insertions formed by repair of a single DSB tend to be shorter and often include short duplications of the sequence immediately surrounding the break site. However, it is possible to obtain large insertions, and in these cases, the inserted sequence has often been traced to other regions of the genome or to plasmid DNA present in the cells.

Indel mutations—and genome editing systems configured to produce indels—are useful for interrupting target sequences, for example, when the generation of a specific final sequence is not required and/or where a frameshift mutation would be tolerated. They can also be useful in settings where particular sequences are preferred, insofar as the certain sequences desired tend to occur preferentially from the repair of an SSB or DSB at a given site. Indel mutations are also a useful tool for evaluating or screening the activity of particular genome editing systems and their components. In these and other settings, indels can be characterized by (a) their relative and absolute frequencies in the genomes of cells contacted with genome editing systems and (b) the distribution of numerical differences relative to the unedited sequence, e.g., ±1, ±2, ±3, etc. As one example, in a lead-finding setting, multiple gRNAs can be screened to identify those gRNAs that most efficiently drive cutting at a target site based on an indel readout under controlled conditions. Guides that produce indels at or above a threshold frequency, or that produce a particular distribution of indels, can be selected for further study and development. Indel frequency and distribution can also be useful as a readout for evaluating different genome editing system implementations or formulations and delivery methods, for instance by keeping the gRNA constant and varying certain other reaction conditions or delivery methods.

Multiplex Strategies

While exemplary strategies discussed above have focused on repair outcomes mediated by single DSBs, genome editing systems according to this disclosure may also be employed to generate two or more DSBs, either in the same locus or in different loci. Strategies for editing that involve the formation of multiple DSBs, or SSBs, are described in, for instance, Cotta-Ramusino.

### Target Cells

The systems and methods described herein provide a streamlined approach for the detection and quantitative measurement of all possible gene editing events at a particular cut site, including targeted integration events, insertions, and deletions. These systems and methods rely on the use of donor templates comprising a 5′ homology arm, a cargo, one or more priming sites, a 3′ homology arm, and optionally a stuffer sequence. Such systems and methods can be used to edit virtually any cell type, at any target locus amenable to genome editing.

Genome editing systems according to this disclosure can be used to manipulate or alter a cell, e.g., to edit or alter a target nucleic acid. The manipulating can occur, in various embodiments, in vivo or ex vivo. In one embodiment, genome editing systems described herein are used to alter a nucleic acid in a target cell in vitro. In another embodiment, genome editing systems described herein are used to alter a nucleic acid in a target cell ex vivo. In another embodiment, genome editing systems described herein are used to alter a nucleic acid in a target cell in vivo.

A variety of cell types can be manipulated or altered according to the embodiments of this disclosure, and in some cases, such as in vivo applications, a plurality of cell types are altered or manipulated, for example by delivering genome editing systems according to this disclosure to a plurality of cell types. In other cases, however, it may be desirable to limit manipulation or alteration to a particular cell type or types. For instance, it can be desirable in some instances to edit a cell with limited differentiation potential or a terminally differentiated cell, such as a photoreceptor cell in the case of Maeder, in which modification of a genotype is expected to result in a change in cell phenotype. In other cases, however, it may be desirable to edit a less differentiated, multipotent or pluripotent, stem or progenitor cell. By way of example, the cell may be an embryonic stem cell, induced pluripotent stem cell (iPSC), hematopoietic stem/progenitor cell (HSPC), or other stem or progenitor cell type that differentiates into a cell type of relevance to a given application or indication.

In certain embodiments, the cell being manipulated is a bacterial cell. In other embodiments, the cell is an archaeal cell. In certain embodiments, the cell being manipulated is a eukaryotic cell. In one embodiment, the cell is a eukaryotic single-cell organism, e.g., an algal cell. In one embodiment, the cell is an animal cell. In one embodiment, the cell is an invertebrate cell, e.g., a fly cell or a worm cell. In another embodiment, the cell is a vertebrate cell. For example, but not by way of limitation, the cell can be a mammalian cell, a rodent cell, a mouse cell, a rat cell, a goat cell, a pig cell, a bird cell, a chicken cell, a turkey cell, a cow cell, a horse cell, a sheep cell, a pig cell, a fish cell, or a frog cell. In one embodiment, the cell is a non-human primate cell. In one embodiment, the cell is a human cell. In certain embodiments, the cell being manipulated is a somatic cell, a germ cell, or a prenatal cell. In certain embodiments, the cell being manipulated is a zygotic cell, a blastocyst cell, an embryonic cell, a stem cell, a mitotically competent cell, or a meiotically competent cell. In certain embodiments, the cell being manipulated is not part of a human embryo. In certain embodiments, the cell being manipulated is a T cell, a CD8⁺ T cell, a CD8⁺ naïve T cell, a CD4⁺ central memory T cell, a CD8⁺ central memory T cell, a CD4⁺ effector memory T cell, a CD4⁺ effector memory T cell, a CD4⁺ T cell, a CD4⁺ stem cell memory T cell, a CD8⁺ stem cell memory T cell, a CD4⁺ helper T cell, a regulatory T cell, a cytotoxic T cell, a natural killer T cell, a CD4⁺ naïve T cell, a TH17 CD4⁺ T cell, a TH1 CD4⁺ T cell, a TH2 CD4⁺ T cell, a TH9 CD4⁺ T cell, a CD4⁺ Foxp3⁺ T cell, a CD4⁺ CD25⁺ CD127⁻ T cell, a CD4⁺ CD25⁺ CD127⁻ Foxp3⁺ T cell. In certain embodiments, the cell being manipulated is a long term hematopoietic stem cell, a short term hematopoietic stem cell, a multipotent progenitor cell, a lineage restricted progenitor cell, a lymphoid progenitor cell, a myeloid progenitor cell, a common myeloid progenitor cell, an erythroid progenitor cell, a megakaryocyte erythroid progenitor cell, a retinal cell, a photoreceptor cell, a rod cell, a cone cell, a retinal pigmented epithelium cell, a trabecular meshwork cell, a cochlear hair cell, an outer hair cell, an inner hair cell, a pulmonary epithelial cell, a bronchial epithelial cell, an alveolar epithelial cell, a pulmonary epithelial progenitor cell, a striated muscle cell, a cardiac muscle cell, a muscle satellite cell, a neuron, a neuronal stem cell, a mesenchymal stem cell, an induced pluripotent stem (iPS) cell, an embryonic stem cell, a monocyte, a megakaryocyte, a neutrophil, an eosinophil, a basophil, a mast cell, a reticulocyte, a B cell, e.g., a progenitor B cell, a Pre B cell, a Pro B cell, a memory B cell, a plasma B cell, a gastrointestinal epithelial cell, a biliary epithelial cell, a pancreatic ductal epithelial cell, an intestinal stem cell, a hepatocyte, a liver stellate cell, a Kupffer cell, an osteoblast, an osteoclast, an adipocyte, a preadipocyte, a pancreatic islet cell (e.g., a beta cell, an alpha cell, a delta cell), a pancreatic exocrine cell, a Schwann cell, or an oligodendrocyte.

In certain embodiments, the target cell is a circulating blood cell, e.g., a reticulocyte, megakaryocyte erythroid progenitor (MEP) cell, myeloid progenitor cell (CMP/GMP), lymphoid progenitor (LP) cell, hematopoietic stem/progenitor cell (HSC), or endothelial cell (EC). In certain embodiments, the target cell is a bone marrow cell (e.g., a reticulocyte, an erythroid cell (e.g., erythroblast), an MEP cell, myeloid progenitor cell (CMP/GMP), LP cell, erythroid progenitor (EP) cell, HSC, multipotent progenitor (MPP) cell, endothelial cell (EC), hemogenic endothelial (HE) cell, or mesenchymal stem cell). In certain embodiments, the target cell is a myeloid progenitor cell (e.g., a common myeloid progenitor (CMP) cell or granulocyte macrophage progenitor (GMP) cell). In certain embodiments, the target cell is a lymphoid progenitor cell, e.g., a common lymphoid progenitor (CLP) cell. In certain embodiments, the target cell is an erythroid progenitor cell (e.g., an MEP cell). In certain embodiments, the target cell is a hematopoietic stem/progenitor cell (e.g., a long term HSC (LT-HSC), short term HSC (ST-HSC), MPP cell, or lineage restricted progenitor (LRP) cell). In certain embodiments, the target cell is a CD34⁺ cell, CD34⁺CD90⁺ cell, CD34⁺CD38⁻ cell, CD34⁺CD90⁺CD49f⁺CD38⁻CD45RA⁻ cell, CD105⁺ cell, CD31⁺, or CD133⁺ cell, or a CD34⁺CD90⁺ CD133⁺ cell. In certain embodiments, the target cell is an umbilical cord blood CD34⁺ HSPC, umbilical cord venous endothelial cell, umbilical cord arterial endothelial cell, amniotic fluid CD34⁺ cell, amniotic fluid endothelial cell, placental endothelial cell, or placental hematopoietic CD34⁺ cell. In certain embodiments, the target cell is a mobilized peripheral blood hematopoietic CD34⁺ cell (after the patient is treated with a mobilization agent, e.g., G-CSF or Plerixafor). In certain embodiments, the target cell is a peripheral blood endothelial cell.

In certain embodiments, the manipulated cell is a plant cell, e.g., a monocot or a dicot cell. In one embodiment, the plant cell is maize, wheat, rice, corn or Setaria. In another embodiment, the plant cell is potato, soybean, tomato, tobacco, or Arabidopsis. Accordingly, in some embodiments, the invention provides compositions and methods for gene targeting and/or gene editing in monocot species of plant, or in dicot species of plant. The compositions, systems, and methods described herein are applicable to any plant species, including for example various dicot and monocot crops, such as tomato, cotton, maize (Zea mays), wheat, Arabidopsis thaliana, Medicago truncatula, Solanum lycopersicum, Glycine max, Brachypodium distachyon, Oryza sativa, Sorghum bicolor, or Solanum tuberosum.

As a corollary, the cell being altered or manipulated is, variously, a dividing cell or a non-dividing cell, depending on the cell type(s) being targeted and/or the desired editing outcome.

When cells are manipulated or altered ex vivo, the cells can be used (e.g., administered to a subject) immediately, or they can be maintained or stored for later use. Those of skill in the art will appreciate that cells can be maintained in culture or stored (e.g., frozen in liquid nitrogen) using any suitable method known in the art.

### Target Nucleic Acids

The donor templates, systems, and methods described herein can be used to edit virtually any target gene in a cell. Specific target nucleic acids can be selected for a desired application based on the cell type to be edited and the effect to be achieved.

In some embodiments, the target gene is a mammalian gene. In some embodiments, the target gene is a disease gene, i.e., a gene associated with a disease or disorder. In some embodiments, the target gene is a rodent gene, e.g., a mouse gene or a rat gene. In some embodiments, the target gene is a primate gene, e.g., a human gene. Exemplary mammalian target genes which may be edited using the donor templates, systems, and methods described herein include, but are not limited to, a mammalian β globin gene (HBB), a gamma globin gene (HBG1), a T-cell receptor alpha (TRAC) gene, a T-cell receptor beta (TRBC) gene, a Kruppel-like factor 1 (KLF1) gene, a dystrophin gene (DMD), a CCR5 gene, a CXCR4 gene, a PPPIR12C (AAVS1) gene, a Cystic Fibrosis Transmembrane Conductance Regulator (CFTR) gene, a B-cell lymphoma/leukemia 11 A (BCL11 A) gene, an hypoxanthine phosphoribosyltransferase (HPRT) gene, an albumin gene, a Factor VIII gene, a Factor IX gene, a Leucine-rich repeat kinase 2 (LRRK2) gene, an alpha-1-antitrypsin (A1AT) gene, a Hungtingin (Htt) gene, a Transporter associated with Antigen Processing (TAP) 1 gene, a TAP2 gene, a tapasin gene (TAPBP), a rhodopsin (RHO) gene, a surfactant protein B gene (SFTPB), a programmed cell death 1 (PD1) gene, a Cytotoxic T-Lymphocyte Antigen 4 (CTLA-4) gene, an human leukocyte antigen (HLA) A gene, an HLA B gene, an HLA C gene, an HLA-DPA gene, an HLA-DQ gene, an HLA-DRA gene, a LMP7 gene, a class II major histocompatibility complex transactivator (CUT A) gene, a glucocorticoid receptor gene (GR), an IL2RG gene and an RFX5 gene. Exemplary plant target genes which may be edited using the donor templates, systems, and methods described herein include, but are not limited to, a plant FAD2 gene, a plant FAD3 gene, a plant ZP15 gene, a plant KASII gene, a plant MDH gene, and a plant EPSPS gene.

### Cells and Cell Populations

In one aspect, the invention provides a cell, or population of cells, which comprise a donor template comprising one or more priming sites, e.g., a donor template comprising a cargo, one or two homology arms, and one or more priming sites. Exemplary donor templates comprising one or more priming sites are provided herein.

In one aspect, the invention provides a cell, or population of cells, which comprise a donor template for recombination with a target nucleic acid in the cell, wherein: (a) a first strand of the target nucleic acid comprises, from 5′ to 3′, P1--H1--X--H2--P2, wherein P1 is a first priming site; H1 is a first homology arm; X is a cleavage site; H2 is a second homology arm; and P2 is a second priming site; and wherein (b) a first strand of the donor template comprises, from 5′ to 3′, A1--P2′--N--A2, or A1--N--P1′--A2, wherein A1 is a homology arm that is substantially identical to H1; P2′ is a priming site that is substantially identical to P2; N is a cargo; P1′ is a priming site that is substantially identical to P1; and A2 is a homology arm that is substantially identical to H2. In an exemplary embodiment, the first strand of the donor template comprises, from 5′ to 3′, A1--P2′--N--P1′--A2.

Additional features of the homology arms, the priming sites, and the cargo are provided above.

In one embodiment, the first strand of the donor template further comprises one or more stuffer sequences, e.g., one, two, three, four or more stuffer sequences. For example, the first strand of the donor template can contain, in some embodiments, a first strand comprising, from 5′ to 3′, A1--S1--P2′--N--A2, or A1--N--P1′--S2--A2, where S1 is a first stuffer, and S2 is a second stuffer. In an exemplary embodiment, the first strand of the donor template can comprise, from 5′ to 3′, from 5′ to 3′, A1--S1--P2′--N--P1′--S2--A2. Additional features of the stuffer sequences are described above. In an exemplary embodiment, the stuffer comprises a random or heterologous sequence having a GC content of approximately 40%.

In one embodiment of the foregoing aspects, the donor template is integrated into the genome of the cell, or population of cells.

In one aspect, the invention provides a cell, or population of cells, which comprise an altered nucleic acid, wherein a first strand of the altered nucleic acid comprises, from 5′ to 3′, a first priming site, a first donor homology arm, a cargo, a priming site that is substantially identical to the first priming site, a second donor homology arm, and a second priming site.

In another aspect, the invention provides a cell, or population of cells, which comprise an altered nucleic acid, wherein a first strand of the altered nucleic acid comprises, from 5′ to 3′, a first priming site, a first donor homology arm, a priming site that is substantially identical to a second priming site, a cargo, a second donor homology arm, and the second priming site.

In another aspect, the invention provides a cell, or population of cells, which comprise an altered nucleic acid, wherein a first strand of the altered nucleic acid comprises, from 5′ to 3′, a first priming site, a first donor homology arm, a priming site that is substantially identical to a second priming site, a cargo, a priming site that is substantially identical to the first priming site, a second donor homology arm, and the second priming site.

The altered nucleic acid can optionally comprise one or more stuffer sequences. For example, the altered nucleic acid can comprise a first strand comprising, from 5′ to 3′, a first priming site, a first donor homology arm, a cargo, a priming site that is substantially identical to the first priming site, a first stuffer, a second donor homology arm, and a second priming site. In another embodiment, the altered nucleic acid can comprise a first strand comprising, from 5′ to 3′, a first priming site, a first donor homology arm, a first stuffer, a priming site that is substantially identical to a second priming site, a cargo, a second donor homology arm, and the second priming site. In another embodiment, the altered nucleic acid can comprise a first strand comprising, from 5′ to 3′, a first priming site, a first donor homology arm, a first stuffer, a priming site that is substantially identical to a second priming site, a cargo, a priming site that is substantially identical to the first priming site, a second stuffer, a second donor homology arm, and the second priming site.

In one embodiment of the foregoing aspects, the cell is derived from a cell comprising a nucleic acid having a first strand which comprises, from 5′ to 3′, the first priming site, a first homology arm substantially identical to the first donor homology arm, a cleavage site, a second homology arm substantially identical to the second donor homology arm, and the second priming site.

In one aspect, the invention provides a cell, or population of cells, comprising an altered nucleic acid, wherein the cell, or population of cells is produced by any of the methods disclosed herein.

The cell, or population of cells, can be provided as a therapeutic composition. Accordingly, in one aspect, the invention provides a therapeutic composition comprising the cell, or population of cells, as disclosed herein. The therapeutic composition can further comprise a pharmaceutically acceptable carrier or excipient. This may include a buffer suitable for administration to a subject.

The cell, or population of cells, can be of any cell type, including but not limited to the target cells disclosed herein, for example, eukaryotic cells, mammalian cells, plant cells, etc. In exemplary embodiments, the cell is a mammalian cell, e.g., a mouse, rat, goat, pig, bird, chicken, turkey, cow, horse, sheep, pig, fish, frog, primate, or human cell. In one embodiment, the cell is a human cell.

Exemplary cell types include, but are not limited to, immune cells (e.g., a T cell, a B cell, a natural killer cell, a monocyte, a macrophage cell, or a dendritic cell), stem cells (e.g., an embryonic stem cell, an induced pluripotent stem cell (iPSC), or a hematopoietic stem/progenitor cell (HSPC)), epithelial cells, endothelial cells, and neurons.

In an exemplary embodiment, the cell is a modified T cell comprising an altered TRAC gene. In another exemplary embodiment, the cell is a modified hematopoietic cell, e.g., a modified hematopoietic stem/progenitor cell, comprising an altered HBB gene.

In other exemplary embodiments, the cell is a plant cell. For example, the cell can be a monocot (e.g., maize, wheat, rice, corn, or Setaria), or a dicot (e.g., potato, soybean, tomato, tobacco, or Arabidopsis).

### Implementation of Genome Editing Systems: Delivery, Formulations, and Routes of Administration

As discussed above, the genome editing systems of this disclosure can be implemented in any suitable manner, meaning that the components of such systems, including without limitation the RNA-guided nuclease, gRNA, and optional donor template nucleic acid, can be delivered, formulated, or administered in any suitable form or combination of forms that results in the transduction, expression or introduction of a genome editing system and/or causes a desired repair outcome in a cell, tissue or subject. Tables 3 and 4 set forth several, non-limiting examples of genome editing system implementations. Those of skill in the art will appreciate, however, that these listings are not comprehensive, and that other implementations are possible. With reference to Table 3 in particular, the table lists several exemplary implementations of a genome editing system comprising a single gRNA and an optional donor template. However, genome editing systems according to this disclosure can incorporate multiple gRNAs, multiple RNA-guided nucleases, and other components such as proteins, and a variety of implementations will be evident to the skilled artisan based on the principles illustrated in the table. In the table, [N/A] indicates that the genome editing system does not include the indicated component.

TABLE 3
Genome Editing System Components

| RNA-guided | | Donor | |
|---|---|---|---|
| Nuclease | gRNA | Template | Comments |
| Protein | RNA | [N/A] | An RNA-guided nuclease protein |
| | | | complexed with a gRNA molecule (an |
| | | | RNP complex) |
| Protein | RNA | DNA | An RNA complex as described above |
| | | | plus a single-stranded or double- |
| | | | stranded donor template. |
| Protein | DNA | [N/A] | An RNA-guided nuclease protein plus |
| | | | gRNA transcribed from DNA. |
| Protein | DNA | DNA | An RNA-guided nuclease protein plus |
| | | | gRNA-encoding DNA and a separate |
| | | | DNA donor template. |
| Protein | DNA | | An RNA-guided nuclease protein and |
| | | | a single DNA encoding both a gRNA |
| | | | and a donor template. |
| DNA | | | A DNA or DNA vector encoding an |
| | | | RNA-guided nuclease, a gRNA and a |
| | | | donor template. |
| DNA | DNA | [N/A] | Two separate DNAs, or two separate |
| | | | DNA vectors, encoding the RNA- |
| | | | guided nuclease and the gRNA, |
| | | | respectively. |
| DNA | DNA | DNA | Three separate DNAs, or three |
| | | | separate DNA vectors, encoding the |
| | | | RNA-guided nuclease, the gRNA and |
| | | | the donor template, respectively. |
| DNA | | [N/A] | A DNA or DNA vector encoding an |
| | | | RNA-guided nuclease and a gRNA |
| DNA | | DNA | A first DNA or DNA vector encoding |
| | | | an RNA-guided nuclease and a gRNA, |
| | | | and a second DNA or DNA vector |
| | | | encoding a donor template. |
| DNA | DNA | | A first DNA or DNA vector encoding |
| | | | an RNA-guided nuclease and second |
| | | | DNA or DNA vector encoding a |
| | | | gRNA and a donor template. |
| DNA | | | A first DNA or DNA vector encoding |
| | DNA | | an RNA-guided nuclease and a donor |
| | | | template, and a second DNA or DNA |
| | | | vector encoding a gRNA |
| DNA | | | A DNA or DNA vector encoding an |
| | RNA | | RNA-guided nuclease and a donor |
| | | | template, and a gRNA |

| | | |
|---|---|---|
| RNA | [N/A] | An RNA or RNA vector encoding an |
| | | RNA-guided nuclease and comprising |
| | | a gRNA |
| RNA | DNA | An RNA or RNA vector encoding an |
| | | RNA-guided nuclease and comprising |
| | | a gRNA, and a DNA or DNA vector |
| | | encoding a donor template. |

Table 3 summarizes various delivery methods for the components of genome editing systems, as described herein. Again, the listing is intended to be exemplary rather than limiting.

TABLE 3

| | | Delivery | | | |
|---|---|---|---|---|---|
| | | into Non- | | | Type of |
| | | Dividing | Duration of | Genome | Molecule |
| Delivery Vector/Mode | | Cells | Expression | Integration | Delivered |
| Physical (e.g., | | YES | Transient | NO | Nucleic Acids |
| electroporation, | | | | | and Proteins |
| particle gun, | | | | | |
| Calcium Phosphate | | | | | |
| transfection, cell | | | | | |
| compression or | | | | | |
| squeezing) | | | | | |
| Viral | Retrovirus | NO | Stable | YES | RNA |
| | Lentivirus | YES | Stable | YES/NO | RNA |
| | | | | with | |
| | | | | modifications | |
| | Adenovirus | YES | Transient | NO | DNA |
| | Adeno- | YES | Stable | NO | DNA |
| | Associated Virus | | | | |
| | (AAV) | | | | |
| | Vaccinia Virus | YES | Very | NO | DNA |
| | | | Transient | | |
| | Herpes Simplex | YES | Stable | NO | DNA |
| | Virus | | | | |
| Non-Viral | Cationic | YES | Transient | Depends on | Nucleic Acids |
| | Liposomes | | | what is | and Proteins |
| | | | | delivered | |
| | Polymeric | YES | Transient | Depends on | Nucleic Acids |
| | Nanoparticles | | | what is | and Proteins |
| | | | | delivered | |
| Biological | Attenuated | YES | Transient | NO | Nucleic Acids |
| Non-Viral | Bacteria | | | | |
| Delivery | Engineered | YES | Transient | NO | Nucleic Acids |
| Vehicles | Bacteriophages | | | | |
| | Mammalian | YES | Transient | NO | Nucleic Acids |
| | Virus-like | | | | |
| | Particles | | | | |
| | Biological | YES | Transient | NO | Nucleic Acids |
| | liposomes: | | | | |
| | Erythrocyte | | | | |
| | Ghosts and | | | | |
| | Exosomes | | | | |

Nucleic Acid-Based Delivery of Genome Editing Systems

Nucleic acids encoding the various elements of a genome editing system according to the present disclosure can be administered to subjects or delivered into cells by art-known methods or as described herein. For example, RNA-guided nuclease-encoding and/or gRNA-encoding DNA, as well as donor template nucleic acids can be delivered by, e.g., vectors (e.g., viral or non-viral vectors), non-vector based methods (e.g., using naked DNA or DNA complexes), or a combination thereof.

Nucleic acids encoding genome editing systems or components thereof can be delivered directly to cells as naked DNA or RNA, for instance by means of transfection or electroporation, or can be conjugated to molecules (e.g., N-acetylgalactosamine) promoting uptake by the target cells (e.g., erythrocytes, HSCs). Nucleic acid vectors, such as the vectors summarized in Table 4, can also be used.

Nucleic acid vectors can comprise one or more sequences encoding genome editing system components, such as an RNA-guided nuclease, a gRNA and/or a donor template. A vector can also comprise a sequence encoding a signal peptide (e.g., for nuclear localization, nucleolar localization, or mitochondrial localization), associated with (e.g., inserted into or fused to) a sequence coding for a protein. As one example, a nucleic acid vectors can include a Cas9 coding sequence that includes one or more nuclear localization sequences (e.g., a nuclear localization sequence from SV40).

The nucleic acid vector can also include any suitable number of regulatory/control elements, e.g., promoters, enhancers, introns, polyadenylation signals, Kozak consensus sequences, or internal ribosome entry sites (IRES). These elements are well known in the art, and are described in Cotta-Ramusino.

Nucleic acid vectors according to this disclosure include recombinant viral vectors. Exemplary viral vectors are set forth in Table 4, and additional suitable viral vectors and their use and production are described in Cotta-Ramusino. Other viral vectors known in the art can also be used. In addition, viral particles can be used to deliver genome editing system components in nucleic acid and/or peptide form. For example, “empty” viral particles can be assembled to contain any suitable cargo. Viral vectors and viral particles can also be engineered to incorporate targeting ligands to alter target tissue specificity.

In addition to viral vectors, non-viral vectors can be used to deliver nucleic acids encoding genome editing systems according to the present disclosure. One important category of non-viral nucleic acid vectors are nanoparticles, which can be organic or inorganic. Nanoparticles are well known in the art, and are summarized in Cotta-Ramusino. Any suitable nanoparticle design can be used to deliver genome editing system components or nucleic acids encoding such components. For instance, organic (e.g., lipid and/or polymer) nanoparticles can be suitable for use as delivery vehicles in certain embodiments of this disclosure. Exemplary lipids for use in nanoparticle formulations, and/or gene transfer are shown in Table 5, and Table 6 lists exemplary polymers for use in gene transfer and/or nanoparticle formulations.

TABLE 5
Lipids Used for Gene Transfer

| Lipid | Abbreviation | Feature |
|---|---|---|
| 1,2-Dioleoyl-sn-glycero-3-phosphatidylcholine | DOPC | Helper |
| 1,2-Dioleoyl-sn-glycero-3-phosphatidylethanolamine | DOPE | Helper |
| Cholesterol | | Helper |
| N-[1-(2,3-Dioleyloxy)propyl]N,N,N-trimethylammonium chloride | DOTMA | Cationic |
| 1,2-Dioleoyloxy-3-trimethylammonium-propane | DOTAP | Cationic |
| Dioctadecylamidoglycylspermine | DOGS | Cationic |
| N-(3-Aminopropyl)-N,N-dimethyl-2,3-bis(dodecyloxy)-1- | GAP-DLRIE | Cationic |
| propanaminium bromide | | |
| Cetyltrimethylammonium bromide | CTAB | Cationic |
| 6-Lauroxyhexyl ornithinate | LHON | Cationic |
| 1-(2,3-Dioleoyloxypropy1)-2,4,6-trimethylpyridinium | 2Oc | Cationic |
| 2,3-Dioleyloxy-N-[2(sperminecarboxamido-ethyl]- | DOSPA | Cationic |
| N,N-dimethyl-1-propanaminium trifluoroacetate | | |
| 1,2-Dioleyl-3-trimethylammonium-propane | DOPA | Cationic |
| N-(2-Hydroxyethyl)-N,N-dimethyl-2,3-bis(tetradecyloxy)-1- | MDRIE | Cationic |
| propanaminium bromide | | |
| Dimyristooxypropyl dimethyl hydroxyethyl ammonium bromide | DMRI | Cationic |
| 3β-[N-N',N'-Dimethylaminoethane)-carbamoyl]cholesterol | DC-Chol | Cationic |
| Bis-guanidium-tren-cholesterol | BGTC | Cationic |
| 1,3-Diodeoxy-2-(6-carboxy-spermyl)-propylamide | DOSPER | Cationic |
| Dimethyloctadecylammonium bromide | DDAB | Cationic |
| Dioctadecylamidoglicylspermidin | DSL | Cationic |
| rac-[(2,3-Dioctadecyloxypropyl)(2-hydroxyethyl)]- | CLIP-1 | Cationic |
| dimethylammonium chloride | | |
| rac-[2(2,3-Dihexadecyloxypropyl- | CLIP-6 | Cationic |
| oxymethyloxy)ethyl]trimethylammonium bromide | | |
| Ethyldimyristoylphosphatidylcholine | EDMPC | Cationic |
| 1,2-Distearyloxy-N,N-dimethyl-3-aminopropane | DSDMA | Cationic |
| 1,2-Dimyristoyl-trimethylammonium propane | DMTAP | Cationic |
| O,O'-Dimyristyl-N-lysyl aspartate | DMKE | Cationic |
| 1,2-Distearoyl-sn-glycero-3-ethylphosphocholine | DSEPC | Cationic |
| N-Palmitoyl D-erythro-sphingosyl carbamoyl-spermine | CCS | Cationic |
| N-t-Butyl-N0-tetradecyl-3-tetradecylaminopropionamidine | diC14-amidine | Cationic |
| Octadecenolyoxy[ethyl-2-heptadecenyl-3 hydroxyethyl]- | DOTIM | Cationic |
| imidazolinium chloride | | |
| N1-Cholesteryloxycarbonyl-3,7-diazanonane-1,9-diamine | CDAN | Cationic |
| 2-(3-[Bis(3-amino-propyl)-amino]propylamino)-N- | RPR209120 | Cationic |
| ditetradecylcarbamoylme-ethyl-acetamide | | |
| 1,2-dilinoleyloxy-3-dimethylaminopropane | DLinDMA | Cationic |
| 2,2-dilinoleyl-4-dimethylaminoethyl-[1,3]-dioxolane | DLin-KC2-DMA | Cationic |
| dilinoleyl-methyl-4-dimethylaminobutyrate | DLin-MC3-DMA | Cationic |

TABLE 6
Polymers Used for GeneTransfer

| Polymer | Abbreviation |
|---|---|
| Poly(ethylene)glycol | PEG |
| Polyethylenimine | PEI |
| Dithiobis(succinimidylpropionate) | DSP |
| Dimethyl-3,3'-dithiobispropionimidate | DTBP |
| Poly(ethylene imine) biscarbamate | PEIC |
| Poly(L-lysine) | PLL |
| Histidine modified PLL | |
| Poly(N-vinylpyrrolidone) | PVP |
| Poly(propylenimine) | PPI |
| Poly(amidoamine) | PAMAM |
| Poly(amido ethylenimine) | SS-PAEI |
| Triethylenetetramine | TETA |
| Poly(β-aminoester) | |
| Poly(4-hydroxy-L-proline ester) | PHP |
| Poly(allylamine) | |
| Poly(α-[4-aminobutyl]-L-glycolic acid) | PAGA |
| Poly(D,L-lactic-co-glycolic acid) | PLGA |
| Poly(N-ethyl-4-vinylpyridinium bromide) | |
| Poly(phosphazene)s | PPZ |
| Poly(phosphoester)s | PPE |
| Poly(phosphoramidate)s | PPA |
| Poly(N-2-hydroxypropylmethacrylamide) | pHPMA |
| Poly (2-(dimethylamino)ethyl methacrylate) | pDMAEMA |
| Poly(2-aminoethyl propylene phosphate) | PPE-EA |
| Chitosan | |
| Galactosylated chitosan | |
| N-Dodacylated chitosan | |
| Histone | |
| Collagen | |
| Dextran-spermine | D-SPM |

Non-viral vectors optionally include targeting modifications to improve uptake and/or selectively target certain cell types. These targeting modifications can include e.g., cell specific antigens, monoclonal antibodies, single chain antibodies, aptamers, polymers, sugars (e.g., N-acetylgalactosamine (GalNAc)), and cell penetrating peptides. Such vectors also optionally use fusogenic and endosome-destabilizing peptides/polymers, undergo acid-triggered conformational changes (e.g., to accelerate endosomal escape of the cargo), and/or incorporate a stimuli-cleavable polymer, e.g., for release in a cellular compartment. For example, disulfide-based cationic polymers that are cleaved in the reducing cellular environment can be used.

In certain embodiments, one or more nucleic acid molecules (e.g., DNA molecules) other than the components of a genome editing system, e.g., the RNA-guided nuclease component and/or the gRNA component described herein, are delivered. In certain embodiments, the nucleic acid molecule is delivered at the same time as one or more of the components of the Genome editing system. In certain embodiments, the nucleic acid molecule is delivered before or after (e.g., less than about 30 minutes, 1 hour, 2 hours, 3 hours, 6 hours, 9 hours, 12 hours, 1 day, 2 days, 3 days, 1 week, 2 weeks, or 4 weeks) one or more of the components of the Genome editing system are delivered. In certain embodiments, the nucleic acid molecule is delivered by a different means than one or more of the components of the genome editing system, e.g., the RNA-guided nuclease component and/or the gRNA component, are delivered. The nucleic acid molecule can be delivered by any of the delivery methods described herein. For example, the nucleic acid molecule can be delivered by a viral vector, e.g., an integration-deficient lentivirus, and the RNA-guided nuclease molecule component and/or the gRNA component can be delivered by electroporation, e.g., such that the toxicity caused by nucleic acids (e.g., DNAs) can be reduced. In certain embodiments, the nucleic acid molecule encodes a therapeutic protein, e.g., a protein described herein. In certain embodiments, the nucleic acid molecule encodes an RNA molecule, e.g., an RNA molecule described herein.

Delivery of RNPs and/or RNA Encoding Genome Editing System Components

RNPs (complexes of gRNAs and RNA-guided nucleases, i.e., ribonucleoprotein complexes) and/or RNAs encoding RNA-guided nucleases and/or gRNAs, can be delivered into cells or administered to subjects by art-known methods, some of which are described in Cotta-Ramusino. In vitro, RNA-guided nuclease-encoding and/or gRNA-encoding RNA can be delivered, e.g., by microinjection, electroporation, transient cell compression or squeezing (see, e.g., Lee 2012). Lipid-mediated transfection, peptide-mediated delivery, GalNAc- or other conjugate-mediated delivery, and combinations thereof, can also be used for delivery in vitro and in vivo.

In vitro, delivery via electroporation comprises mixing the cells with the RNA encoding RNA-guided nucleases and/or gRNAs, with or without donor template nucleic acid molecules, in a cartridge, chamber or cuvette and applying one or more electrical impulses of defined duration and amplitude. Systems and protocols for electroporation are known in the art, and any suitable electroporation tool and/or protocol can be used in connection with the various embodiments of this disclosure.

Route of Administration

Genome editing systems, or cells altered or manipulated using such systems, can be administered to subjects by any suitable mode or route, whether local or systemic. Systemic modes of administration include oral and parenteral routes. Parenteral routes include, by way of example, intravenous, intramarrow, intrarterial, intramuscular, intradermal, subcutaneous, intranasal, and intraperitoneal routes. Components administered systemically can be modified or formulated to target, e.g., HSCs, hematopoietic stem/progenitor cells, or erythroid progenitors or precursor cells.

Local modes of administration include, by way of example, intramarrow injection into the trabecular bone or intrafemoral injection into the marrow space, and infusion into the portal vein. In certain embodiments, significantly smaller amounts of the components (compared with systemic approaches) can exert an effect when administered locally (for example, directly into the bone marrow) compared to when administered systemically (for example, intravenously). Local modes of administration can reduce or eliminate the incidence of potentially toxic side effects that may occur when therapeutically effective amounts of a component are administered systemically.

Administration can be provided as a periodic bolus (for example, intravenously) or as continuous infusion from an internal reservoir or from an external reservoir (for example, from an intravenous bag or implantable pump). Components can be administered locally, for example, by continuous release from a sustained release drug delivery device.

In addition, components can be formulated to permit release over a prolonged period of time. A release system can include a matrix of a biodegradable material or a material which releases the incorporated components by diffusion. The components can be homogeneously or heterogeneously distributed within the release system. A variety of release systems can be useful, however, the choice of the appropriate system will depend upon rate of release required by a particular application. Both non-degradable and degradable release systems can be used. Suitable release systems include polymers and polymeric matrices, non-polymeric matrices, or inorganic and organic excipients and diluents such as, but not limited to, calcium carbonate and sugar (for example, trehalose). Release systems may be natural or synthetic. However, synthetic release systems are preferred because generally they are more reliable, more reproducible and produce more defined release profiles. The release system material can be selected so that components having different molecular weights are released by diffusion through or degradation of the material.

Representative synthetic, biodegradable polymers include, for example: polyamides such as poly(amino acids) and poly(peptides); polyesters such as poly(lactic acid), poly(glycolic acid), poly(lactic-co-glycolic acid), and poly(caprolactone); poly(anhydrides); polyorthoesters; polycarbonates; and chemical derivatives thereof (substitutions, additions of chemical groups, for example, alkyl, alkylene, hydroxylations, oxidations, and other modifications routinely made by those skilled in the art), copolymers and mixtures thereof. Representative synthetic, non-degradable polymers include, for example: polyethers such as poly(ethylene oxide), poly(ethylene glycol), and poly(tetramethylene oxide); vinyl polymers-polyacrylates and polymethacrylates such as methyl, ethyl, other alkyl, hydroxyethyl methacrylate, acrylic and methacrylic acids, and others such as poly(vinyl alcohol), poly(vinyl pyrolidone), and poly(vinyl acetate); poly(urethanes); cellulose and its derivatives such as alkyl, hydroxyalkyl, ethers, esters, nitrocellulose, and various cellulose acetates; polysiloxanes; and any chemical derivatives thereof (substitutions, additions of chemical groups, for example, alkyl, alkylene, hydroxylations, oxidations, and other modifications routinely made by those skilled in the art), copolymers and mixtures thereof.

Poly(lactide-co-glycolide) microsphere can also be used. Typically the microspheres are composed of a polymer of lactic acid and glycolic acid, which are structured to form hollow spheres. The spheres can be approximately 15-30 microns in diameter and can be loaded with components described herein.

Multi-Modal or Differential Delivery of Components

Skilled artisans will appreciate, in view of the instant disclosure, that different components of genome editing systems disclosed herein can be delivered together or separately and simultaneously or nonsimultaneously. Separate and/or asynchronous delivery of genome editing system components can be particularly desirable to provide temporal or spatial control over the function of genome editing systems and to limit certain effects caused by their activity.

Different or differential modes as used herein refer to modes of delivery that confer different pharmacodynamic or pharmacokinetic properties on the subject component molecule, e.g., a RNA-guided nuclease molecule, gRNA, template nucleic acid, or payload. For example, the modes of delivery can result in different tissue distribution, different half-life, or different temporal distribution, e.g., in a selected compartment, tissue, or organ.

Some modes of delivery, e.g., delivery by a nucleic acid vector that persists in a cell, or in progeny of a cell, e.g., by autonomous replication or insertion into cellular nucleic acid, result in more persistent expression of and presence of a component. Examples include viral, e.g., adenovirus, AAV, lentivirus, and integration-deficient lentivirus (IDLV), delivery.

By way of example, the components of a genome editing system, e.g., a RNA-guided nuclease and a gRNA, can be delivered by modes that differ in terms of resulting half-life or persistent of the delivered component the body, or in a particular compartment, tissue or organ. In certain embodiments, a gRNA can be delivered by such modes. The RNA-guided nuclease molecule component can be delivered by a mode which results in less persistence or less exposure to the body or a particular compartment or tissue or organ.

More generally, in certain embodiments, a first mode of delivery is used to deliver a first component and a second mode of delivery is used to deliver a second component. The first mode of delivery confers a first pharmacodynamic or pharmacokinetic property. The first pharmacodynamic property can be, e.g., distribution, persistence, or exposure, of the component, or of a nucleic acid that encodes the component, in the body, a compartment, tissue or organ. The second mode of delivery confers a second pharmacodynamic or pharmacokinetic property. The second pharmacodynamic property can be, e.g., distribution, persistence, or exposure, of the component, or of a nucleic acid that encodes the component, in the body, a compartment, tissue or organ.

In certain embodiments, the first pharmacodynamic or pharmacokinetic property, e.g., distribution, persistence or exposure, is more limited than the second pharmacodynamic or pharmacokinetic property.

In certain embodiments, the first mode of delivery is selected to optimize, e.g., minimize, a pharmacodynamic or pharmacokinetic property, e.g., distribution, persistence or exposure.

In certain embodiments, the second mode of delivery is selected to optimize, e.g., maximize, a pharmacodynamic or pharmacokinetic property, e.g., distribution, persistence or exposure.

In certain embodiments, the first mode of delivery comprises the use of a relatively persistent element, e.g., a nucleic acid, e.g., a plasmid or viral vector, e.g., an AAV vector, an adenoviral vector, a lentiviral vector, or an integration-deficient lentiviral vector (IDLV). As such vectors are relatively persistent product transcribed from them would be relatively persistent.

In certain embodiments, the second mode of delivery comprises a relatively transient element, e.g., an RNA or protein.

In certain embodiments, the first component comprises gRNA, and the delivery mode is relatively persistent, e.g., the gRNA is transcribed from a plasmid or viral vector, e.g., an AAV vector, an adenoviral vector, a lentiviral vector, or an integration-deficient lentiviral vector (IDLV). Transcription of these genes would be of little physiological consequence because the genes do not encode for a protein product, and the gRNAs are incapable of acting in isolation. The second component, a RNA-guided nuclease molecule, is delivered in a transient manner, for example as mRNA or as protein, ensuring that the full RNA-guided nuclease molecule/gRNA complex is only present and active for a short period of time.

Furthermore, the components can be delivered in different molecular form or with different delivery vectors that complement one another to enhance safety and tissue specificity.

Use of differential delivery modes can enhance performance, safety, and/or efficacy, e.g., the likelihood of an eventual off-target modification can be reduced. Delivery of immunogenic components, e.g., Cas9 molecules, by less persistent modes can reduce immunogenicity, as peptides from the bacterially-derived Cas enzyme are displayed on the surface of the cell by MHC molecules. A two-part delivery system can alleviate these drawbacks.

Differential delivery modes can be used to deliver components to different, but overlapping target regions. The formation active complex is minimized outside the overlap of the target regions. Thus, in certain embodiments, a first component, e.g., a gRNA is delivered by a first delivery mode that results in a first spatial, e.g., tissue, distribution. A second component, e.g., a RNA-guided nuclease molecule is delivered by a second delivery mode that results in a second spatial, e.g., tissue, distribution. In certain embodiments, the first mode comprises a first element selected from a liposome, nanoparticle, e.g., polymeric nanoparticle, and a nucleic acid, e.g., viral vector. The second mode comprises a second element selected from the group. In certain embodiments, the first mode of delivery comprises a first targeting element, e.g., a cell specific receptor or an antibody, and the second mode of delivery does not include that element. In certain embodiments, the second mode of delivery comprises a second targeting element, e.g., a second cell specific receptor or second antibody.

When the RNA-guided nuclease molecule is delivered in a virus delivery vector, a liposome, or polymeric nanoparticle, there is the potential for delivery to and therapeutic activity in multiple tissues, when it may be desirable to only target a single tissue. A two-part delivery system can resolve this challenge and enhance tissue specificity. If the gRNA and the RNA-guided nuclease molecule are packaged in separated delivery vehicles with distinct but overlapping tissue tropism, the fully functional complex is only be formed in the tissue that is targeted by both vectors.

### Determination of Targeted Integration Frequency in a Cell Population

The genome editing systems and methods described herein may advantageously be used to analyze a component of genome editing system (e.g., a nuclease, a gRNA, or a donor template), or variants thereof, to determine the component's suitability for generating targeted integration outcomes. In some embodiments, a genome editing system described herein may be used to determining the frequency of targeted integration versus the frequency of non-targeted integration in a population of cells treated with the genome editing system.

For example, when the genome editing system is used to alter a population of cells, wherein each cell comprises a target nucleic acid comprising from 5′ to 3′, P1--H1--X--H2--P2, wherein P1 is a first priming site; H1 is a first homology arm; X is the cleavage site; H2 is a second homology arm; and P2 is a second priming site; and the donor template comprises from 5′ to 3′, A1--P2′--N--P1′--A2, wherein A1 is a homology arm that is substantially identical to H1; P2′ is a priming site that is substantially identical to P2; N is a cargo; P1′ is a priming site that is substantially identical to P1; and A2 is a homology arm that is substantially identical to H2; then the overall targeted integration frequency may be calculated according to the following formula:

$$\\ Sequencing ( Overall ) = \frac{Average ( AmpY + AmpZ )}{AmpX + Average ( AmpY + AmpZ )} \times 100$$

wherein AmpY is the amplicon generated from the amplification of the 5′ junction at a targeted integration event, AmpZ is generated from the amplification of the 3′ junction at a targeted integration event, and AmpX is generated from the amplification of a non-targeted integration event.

When the genome editing system is used to alter a population of cells, wherein each cell comprises a target nucleic acid comprising from 5′ to 3′, P1--H1--X--H2--P2, wherein P1 is a first priming site; H1 is a first homology arm; X is the cleavage site; H2 is a second homology arm; and P2 is a second priming site; and the donor template comprises from 5′ to 3′, A1--P2′--N--A2, wherein A1 is a homology arm that is substantially identical to H1; P2′ is a priming site that is substantially identical to P2; N is a cargo; and A2 is a homology arm that is substantially identical to H2; then the overall targeted integration frequency may be calculated according to the following formula:

$$\\ Sequencing ( {{5}^{}}^{′} ) = \frac{AmpY}{AmpX + AmpY} \times 100$$

wherein AmpY is the amplicon generated from the amplification of the 5′ junction at a targeted integration event, and AmpX is generated from the amplification of a non-targeted integration event.

When the genome editing system is used to alter a population of cells, wherein each cell comprises a target nucleic acid comprising from 5′ to 3′, P1--H1--X--H2--P2, wherein P1 is a first priming site; H1 is a first homology arm; X is the cleavage site; H2 is a second homology arm; and P2 is a second priming site; and the donor template comprises from 5′ to 3′, A1--N--P1′--A2, wherein A1 is a homology arm that is substantially identical to H1; N is a cargo; P1′ is a priming site that is substantially identical to P1; and A2 is a homology arm that is substantially identical to H2; then the overall targeted integration frequency may be calculated according to the following formula:

$$\\ Sequencing ( {{3}^{}}^{′} ) = \frac{AmpZ}{AmpX + AmpZ} \times 100$$

wherein AmpZ is generated from the amplification of the 3′ junction at a targeted integration event, and AmpX is generated from the amplification of a non-targeted integration event.

Other methods for detecting gene editing outcomes, e.g., targeted integration events, include Digital Droplet PCR (ddPCR) and UDITAS™.

ddPCR can be used to quantitatively detect targeted integration events. In this method, droplets are formed in a water/oil emulsion to partition template DNA molecules, and PCR amplification is carried out within each droplet. A fluorescent probe anneals to the amplified product, and is read by a droplet analyzer. ddPCR is capable of providing an absolute count of target DNA molecules in a given sample. To detect gene editing outcomes, a detection probe that anneals to the amplified product is designed to detect the predicted amplification products.

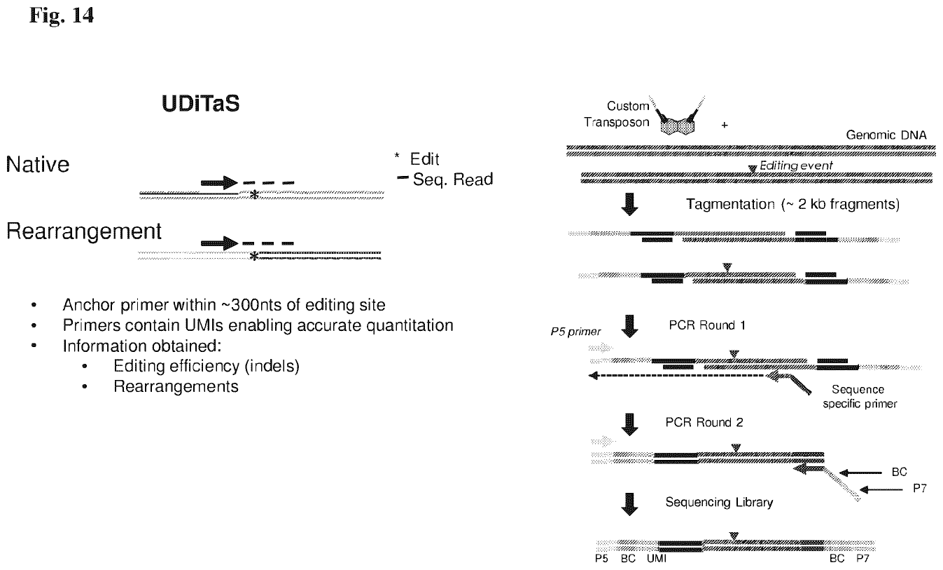

Another PCR-based method, Uni-Directional Targeted Sequencing (“UDITAS™”), can detect the presence of genomic modifications, e.g., indels and targeted integration events, without specific knowledge regarding the nature of the alteration. In the UDITAS™ method, genomic DNA that has been cleaved, modified, and/or edited as described herein is contacted with a transposon under conditions (e.g., in the presence of a transposase) whereby the transposon is inserted into the nucleic acid template. Such transposition reactions and conditions are known in the art (see, e.g., U.S. Pat. Nos. 6,593,113 and 9,080,211). In some embodiments, transposition conditions are selected with the desired fragment size in mind. The transposition reaction results in fragmentation of the nucleic acid template into a plurality of tagmented double-stranded nucleic acid fragments, where the 3′ end of the transferred strand of the transposon is attached to the 5′ end of the nucleic acid fragments. The transferred strand of the transposon comprises a first detection sequence at the 5′ end of the transferred strand. Following the transposition reaction, the tagmented nucleic acid fragments are amplified, e.g., using PCR, using a set of primers. A first primer can be a fixed primer, comprising a nucleotide sequence complementary to a predetermined location in the genomic DNA. A first primer can also be a fixed primer, comprising a nucleotide sequence complementary to at least a portion of a double-stranded oligonucleotide as described herein. The first primer also includes a second detection sequence at its 5′ end. A second primer is a selective primer, comprising a nucleotide sequence complementary to at least a portion of the first detection sequence. The amplification forms amplified nucleic acid fragments, which include (in 5′ to 3′ orientation): the first detection sequence, the transferred strand of the transposon attached to the 5′ end of the nucleic acid fragments, and the second detection sequence. The amplified nucleic acid fragments can then be sequenced. For example, the first and second detection sequences can include sequencing tags described herein to facilitate sequencing. In some embodiments, the method can include a size separation step after tagmentation and before PCR. This method is depicted in FIG. 14, and is described in PCT/US18/12652, incorporated herein by reference in its entirety.

### EXAMPLES

The following Examples are merely illustrative and are not intended to limit the scope or content of the invention in any way.

### Example 1: Targeted Integration at the HBB Locus

Previously, it was thought that longer homology arms provided more efficient homologous recombination, and typical homology arm lengths were between 500 and 2000 bases (Wang et al., NAR 2015; De Ravin, et al. NBT 2016; Genovese et al. Nature 2014). However, the methods described in the instant example can surprisingly be performed using donor templates having a shorter homology arm (HA) to achieve targeted integration.

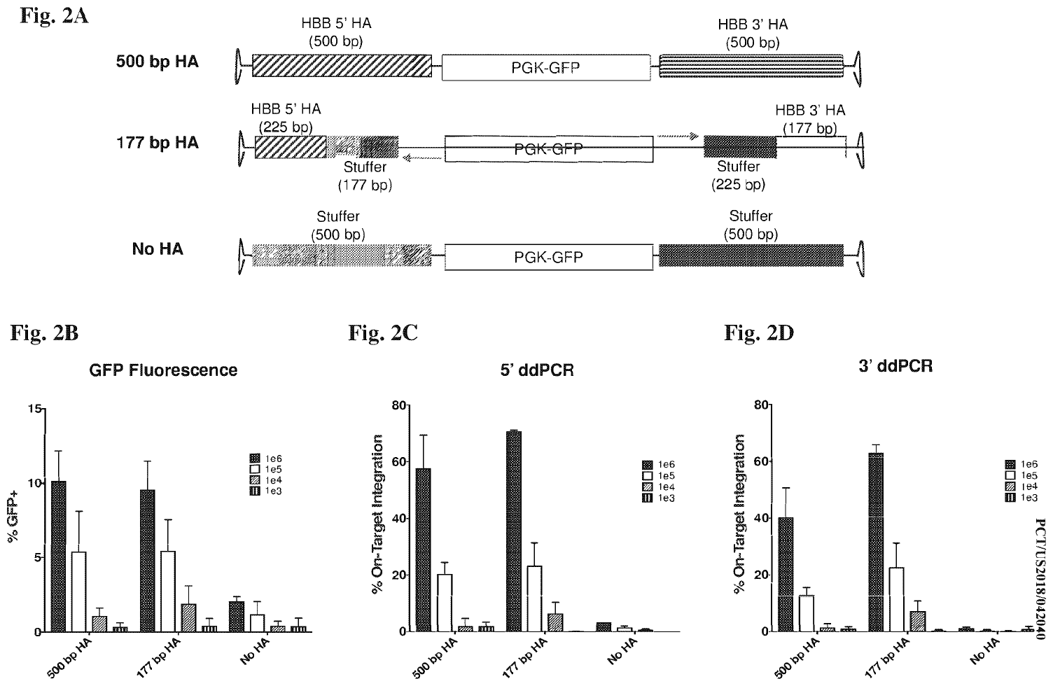

To test whether shortening the homology arms negatively impacted targeted integration efficiency, two AAV6 donor templates to the HBB locus were designed (FIG. 2A). The first donor template contained symmetrical homology arms of 500 nt each, flanking a GFP expression cassette (hPGK promoter, GFP, and polyA sequence). The second donor template contained shorter homology arms (5′: 225 bp, 3′: 177 bp) in addition to stuffer DNA and the genomic priming sites, as described above, flanking an identical GFP cassette. A third donor template having 500 nt of DNA that was non-homologous to the human genome 5′ and 3′ of the same GFP cassette was used. The 5′ and 3′ stuffer sequences were derived from the master stuffer sequence and comprised different sequences in each construct to avoid intramolecular recombination.

Table 7 provides the sequences for the master stuffer and the three donor templates depicted in FIG. 2A. A “master stuffer sequence” consists of 2000 nucleotides. It contains roughly the same GC content as the genome as a whole. (e.g., ˜40% for the whole genome). Depending on the target locus, the GC content may vary. Based on the design of the donor templates, certain portions of the “master stuffer sequence” (or the reverse compliment thereof) are selected as appropriate stuffers. The selection is based on the following three criteria:
1) the length
2) the homology, and
3) structure.

In the second exemplary donor template design depicted in FIG. 2A (HA+Stuffers), the stuffer 5′ to the cargo is 177 nucleotides long while the stuffer 3′ to the cargo is 225 nucleotides long. Therefore, the 5′ stuffer (177nt) may be any consecutive 177 nucleotide sequence within the “master stuffer sequence” or the reverse compliment thereof. The 3′ stuffer (225 nt) may be any consecutive 225 nucleotide sequence within the “master stuffer sequence”, or the reverse compliment thereof.

For the homology requirement, neither the 5′ stuffer nor the 3′ stuffer have homology with any other sequence in the genome (e.g., no more than 20 nucleotide homology), nor to any other sequence in the donor template (i.e., primers, cargo, the other stuffer sequence, homology arms). It is preferable that the stuffer not contain a nucleic acid sequence that forms secondary structures.

TABLE 7
Nucleic Acid Sequences for the Master Stuffer and Donor Templates.

| | | SEQ |
|---|---|---|
| | | ID |
| DESCRIPTION | SEQUENCE | NO. |
| Master Stuffer | TACTCTTAATTCATTACATATTGTGCGGTCGAATTCAGGGAGC | 102 |
| | CGATAATGCGGTTACAATAATTCCTATACTTAAATATACAAAG | |
| | ATTTAAAATTTCAAAAAATGGTTACCAGCATCGTTAGTGCGTA | |
| | TACATCAAGAGGCACGTGCCCCGGAGACAGCAAGTAAGCTCT | |
| | TTAAACATGCTTTGACATACGATTTTTAATAAAACATGAGCAT | |
| | TTGAATAAAAACGACTTCCTCATACTGTAAACATCACGCATGC | |
| | ACATTAGACAATAATCCAGTAACGAAACGGCTTCAGTCGTAAT | |
| | CGCCCATATAGTTGGCTACAGAATGTTGGATAGAGAACTTAAG | |
| | TACGCTAAGGCGGCGTATTTTCTTAATATTTAGGGGTATTGCC | |
| | GCAGTCATTACAGATAACCGCCTATGCGGCCATGCCAGGATTA | |
| | TAGATAACTTTTTAACATTAGCCGCAGAGGTGGGACTAGCACG | |
| | TAATATCAGCACATAACGTGTCAGTCAGCATATTACGGAATAA | |
| | TCCTATCGTTATCAGATCTCCCCTGTCATATCACAACATGTTTC | |
| | GATGTTCCAAAACCGGGAACATTTTGGATCGGTTAAATGATTG | |
| | TACATCATTTGTTGCAGACCTTAGGAACATCCATCATCCGCCG | |
| | CCCTTCATCTCTCAAAGTTATCGCTTGTAAATGTATCACAACTA | |
| | GTATGGTGTAAAATATAGTACCCGATAGACTCGATTTAGGCTG | |
| | TGAGGTTAGTAACTCTAACTTGTGCTTTCGACACAGATCCTCGT | |
| | TTCATGCAAATTTAATTTTGCTGGCTAGATATATCAATCGTTCG | |
| | ATTATTCAGAGTTTTGGTGAGGAGCCCCCTCAGATGGGAGCAT | |
| | TTTCACTACTTTAAAGAATAACGTATTTTTCGCCCTGTCCCTTA | |
| | GTGACTTAAAAAGAATGGGGGCTAGTGCTTAGAGCTGGTAGG | |
| | GCTTTTTGGTTCTATCTGTTAAGCGAATAAGCTGTCACCTAAGC | |
| | AAATTAATGCTTTCATTGTACCCCGGAACTTTAAATCTATGAA | |
| | CAATCGCAACAAATTGTCCAAAGGCAACAATACGACACAGTT | |
| | AGAGGCCATCGGCGCAGGTACACTCTATCCACGCCTATCAGAA | |
| | TGTCACCTGGTTAATGGTCAATTTAGGTGGCTGGAGGCACATG | |
| | TGAAGCAATATGGTCTAGGGAAAGATATCGGTTTACTTAGATT | |
| | TTATAGTTCCGGATCCAACTTAAATAATATAGGTATTAAAGAG | |
| | CAGTATCAAGAGGGTTTCTTCCCAAGGAATCTTGCGATTTTCA | |
| | TACACAGCTTTAACAAATTTCACTAGACGCACCTTCATTTTGTC | |
| | GTCTCGTTGTATATGAGTCCGGGGTAAGAATTTTTTACCGTATT | |
| | TAACATGATCAACGGGTACTAAAGCAATGTCATTTCTAAACAC | |
| | AGTAGGTAAAGGACACGTCATCTTATTTTAAAGAATGTCAGAA | |
| | ATCAGGGAGACTAGATCGATATTACGTGTTTTTTGAGTCAAAG | |
| | ACGGCCGTAAAATAATCAAGCAGTCTTTCTACCTGTACTTGTC | |
| | GCTACCTAGAATCTTTAATTTATCCATGTCAAGGAGGATGCCC | |
| | ATCTGAAACAATACCTGTTGCTAGATCGTCTAACAACGGCATC | |
| | TTGTCGTCCATGCGGGGTTGTTCTTGTACGTATCAGCGTCGGTT | |
| | ATATGTAAAAATAATGTTTTACTACTATGCCATCTGTCCCGTAT | |
| | TCTTAAGCATGACTAATATTAAAAGCCGCCTATATATCGAGAA | |
| | CGACTACCATTGGAATTTAAAATTGCTTCCAAGCTATGATGAT | |
| | GTGACCTCTCACATTGTGGTAGTATAAACTATGGTTAGCCACG | |
| | ACTCGTTCGGACAAGTAGTAATATCTGTTGGTAATAGTCGGGT | |
| | TACCGCGAAATATTTGAAATTGATATTAAGAAGCAATGATTTG | |
| | TACATAAGTATACCTGTAATGAATTCCTGCGTTAGCAGCTTAG | |
| | TATCCATTATTAGAG | |
| Donor template | TTATCCCCTTCCTATGACATGAACTTAACCATAGAAAAGAAGG | 103 |
| design 1 (HA | GGAAAGAAAACATCAAGCGTCCCATAGACTCACCCTGAAGTT | |
| only) | CTCAGGATCCACGTGCAGCTTGTCACAGTGCAGCTCACTCAGT | |
| | GTGGCAAAGGTGCCCTTGAGGTTGTCCAGGTGAGCCAGGCCAT | |
| | CACTAAAGGCACCGAGCACTTTCTTGCCATGAGCCTTCACCTT | |
| | AGGGTTGCCCATAACAGCATCAGGAGTGGACAGATCCCCAAA | |
| | GGACTCAAAGAACCTCTGGGTCCAAGGGTAGACCACCAGCAG | |
| | CCTAAGGGTGGGAAAATAGACCAATAGGCAGAGAGAGTCAGT | |
| | GCCTATCAGAAACCCAAGAGTCTTCTCTGTCTCCACATGCCCA | |
| | GTTTCTATTGGTCTCCTTAAACCTGTCTTGTAACCTTGATACCA | |
| | ACCTGCCCAGGGCCTCACCACCAACTTCATCCACGTTCACCTT | |
| | GCCCCACAGGGCAGTAACGGCAGACTTCTCAAGCTTCCATAGA | |
| | GCCCACCGCATCCCCAGCATGCCTGCTATTGTCTTCCCAATCCT | |
| | CCCCCTTGCTGTCCTGCCCCACCCCACCCCCCAGAATAGAATG | |
| | ACACCTACTCAGACAATGCGATGCAATTTCCTCATTTTATTAG | |
| | GAAAGGACAGTGGGAGTGGCACCTTCCAGGGTCAAGGAAGGC | |
| | ACGGGGGAGGGGCAAACAACAGATGGCTGGCAACTAGAAGGC | |
| | ACAGTCGAGGCTGATCAGCGGGTTTAAACGGGCCCTCTAGACT | |
| | CGACGCGGCCGCTTTACTTGTACAGCTCGTCCATGCCGAGAGT | |
| | GATCCCGGCGGCGGTCACGAACTCCAGCAGGACCATGTGATC | |
| | GCGCTTCTCGTTGGGGTCTTTGCTCAGGGCGGACTGGGTGCTC | |
| | AGGTAGTGGTTGTCGGGCAGCAGCACGGGGCCGTCGCCGATG | |
| | GGGGTGTTCTGCTGGTAGTGGTCGGCGAGCTGCACGCTGCCGT | |
| | CCTCGATGTTGTGGCGGATCTTGAAGTTCACCTTGATGCCGTTC | |
| | TTCTGCTTGTCGGCCATGATATAGACGTTGTGGCTGTTGTAGTT | |
| | GTACTCCAGCTTGTGCCCCAGGATGTTGCCGTCCTCCTTGAAGT | |
| | CGATGCCCTTCAGCTCGATGCGGTTCACCAGGGTGTCGCCCTC | |
| | GAACTTCACCTCGGCGCGGGTCTTGTAGTTGCCGTCGTCCTTG | |
| | AAGAAGATGGTGCGCTCCTGGACGTAGCCTTCGGGCATGGCG | |
| | GACTTGAAGAAGTCGTGCTGCTTCATGTGGTCGGGGTAGCGGC | |
| | TGAAGCACTGCACGCCGTAGGTCAGGGTGGTCACGAGGGTGG | |
| | GCCAGGGCACGGGCAGCTTGCCGGTGGTGCAGATGAACTTCA | |
| | GGGTCAGCTTGCCGTAGGTGGCATCGCCCTCGCCCTCGCCGGA | |
| | CACGCTGAACTTGTGGCCGTTTACGTCGCCGTCCAGCTCGACC | |
| | AGGATGGGCACCACCCCGGTGAACAGCTCCTCGCCCTTGCTCA | |
| | CCATGGTGGCGACCGGTGGGGAGAGAGGTCGGTGATTCGGTC | |
| | AACGAGGGAGCCGACTGCCGACGTGCGCTCCGGAGGCTTGCA | |
| | GAATGCGGAACACCGCGCGGGCAGGAACAGGGCCCACACTAC | |
| | CGCCCCACACCCCGCCTCCCGCACCGCCCCTTCCCGGCCGCTG | |
| | CTCTCGGCGCGCCCTGCTGAGCAGCCGCTATTGGCCACAGCCC | |
| | ATCGCGGTCGGCGCGCTGCCATTGCTCCCTGGCGCTGTCCGTC | |
| | TGCGAGGGTACTAGTGAGACGTGCGGCTTCCGTTTGTCACGTC | |
| | CGGCACGCCGCGAACCGCAAGGAACCTTCCCGACTTAGGGGC | |
| | GGAGCAGGAAGCGTCGCCGGGGGGCCCACAAGGGTAGCGGCG | |
| | AAGATCCGGGTGACGCTGCGAACGGACGTGAAGAATGTGCGA | |
| | GACCCAGGGTCGGCGCCGCTGCGTTTCCCGGAACCACGCCCAG | |
| | AGCAGCCGCGTCCCTGCGCAAACCCAGGGCTGCCTTGGAAAA | |
| | GGCGCAACCCCAACCCCGTGGAAGCTCTCAGGAGTCAGATGC | |
| | ACCATGGTGTCTGTTTGAGGTTGCTAGTGAACACAGTTGTGTC | |
| | AGAAGCAAATGTAAGCAATAGATGGCTCTGCCCTGACTTTTAT | |
| | GCCCAGCCCTGGCTCCTGCCCTCCCTGCTCCTGGGAGTAGATT | |
| | GGCCAACCCTAGGGTGTGGCTCCACAGGGTGAGGTCTAAGTG | |
| | ATGACAGCCGTACCTGTCCTTGGCTCTTCTGGCACTGGCTTAG | |
| | GAGTTGGACTTCAAACCCTCAGCCCTCCCTCTAAGATATATCT | |
| | CTTGGCCCCATACCATCAGTACAAATTGCTACTAAAAACATCC | |
| | TCCTTTGCAAGTGTATTTACGTAATATTTGGAATCACAGCTTGG | |
| | TAAGCATATTGAAGATCGTTTTCCCAATTTTCTTATTACACAAA | |
| | TAAGAAGTTGATGCACTAAAAGTGGAAGAGTTTTGTCTACCAT | |
| | AATTCAGCTTTGGGATATGTAGATGGATCTCTTCCTGCGTCTCC | |
| | AGAATATGC | |
| Donor template | GTCCAAGGGTAGACCACCAGCAGCCTAAGGGTGGGAAAATAG | 104 |
| design 2 | ACCAATAGGCAGAGAGAGTCAGTGCCTATCAGAAACCCAAGA | |
| (HA + Stuffers) | GTCTTCTCTGTCTCCACATGCCCAGTTTCTATTGGTCTCCTTAA | |
| | ACCTGTCTTGTAACCTTGATACCAACCTGCCCAGGGCCTCACC | |
| | ACCAACTTCATCCACGTTCACCTTGCCCCACAGGGCAGTAACG | |
| | GCAGACTTCTCTACTCTTAATTCATTACATATTGTGCGGTCGAA | |
| | TTCAGGGAGCCGATAATGCGGTTACAATAATTCCTATACTTAA | |
| | ATATACAAAGATTTAAAATTTCAAAAAATGGTTACCAGCATCG | |
| | TTAGTGCGTATACATCAAGAGGCACGTGCCCCGGAGACAGCA | |
| | AGTAAGCTCTTTAAACGGTCTAAGTGATGACAGCCGTAAGCTT | |
| | CCATAGAGCCCACCGCATCCCCAGCATGCCTGCTATTGTCTTC | |
| | CCAATCCTCCCCCTTGCTGTCCTGCCCCACCCCACCCCCCAGAA | |
| | TAGAATGACACCTACTCAGACAATGCGATGCAATTTCCTCATT | |
| | TTATTAGGAAAGGACAGTGGGAGTGGCACCTTCCAGGGTCAA | |
| | GGAAGGCACGGGGGAGGGGCAAACAACAGATGGCTGGCAACT | |
| | AGAAGGCACAGTCGAGGCTGATCAGCGGGTTTAAACGGGCCC | |
| | TCTAGACTCGACGCGGCCGCTTTACTTGTACAGCTCGTCCATG | |
| | CCGAGAGTGATCCCGGCGGCGGTCACGAACTCCAGCAGGACC | |
| | ATGTGATCGCGCTTCTCGTTGGGGTCTTTGCTCAGGGCGGACT | |
| | GGGTGCTCAGGTAGTGGTTGTCGGGCAGCAGCACGGGGCCGT | |
| | CGCCGATGGGGGTGTTCTGCTGGTAGTGGTCGGCGAGCTGCAC | |
| | GCTGCCGTCCTCGATGTTGTGGCGGATCTTGAAGTTCACCTTG | |
| | ATGCCGTTCTTCTGCTTGTCGGCCATGATATAGACGTTGTGGCT | |
| | GTTGTAGTTGTACTCCAGCTTGTGCCCCAGGATGTTGCCGTCCT | |
| | CCTTGAAGTCGATGCCCTTCAGCTCGATGCGGTTCACCAGGGT | |
| | GTCGCCCTCGAACTTCACCTCGGCGCGGGTCTTGTAGTTGCCG | |
| | TCGTCCTTGAAGAAGATGGTGCGCTCCTGGACGTAGCCTTCGG | |
| | GCATGGCGGACTTGAAGAAGTCGTGCTGCTTCATGTGGTCGGG | |
| | GTAGCGGCTGAAGCACTGCACGCCGTAGGTCAGGGTGGTCAC | |
| | GAGGGTGGGCCAGGGCACGGGCAGCTTGCCGGTGGTGCAGAT | |
| | GAACTTCAGGGTCAGCTTGCCGTAGGTGGCATCGCCCTCGCCC | |
| | TCGCCGGACACGCTGAACTTGTGGCCGTTTACGTCGCCGTCCA | |
| | GCTCGACCAGGATGGGCACCACCCCGGTGAACAGCTCCTCGCC | |
| | CTTGCTCACCATGGTGGCGACCGGTGGGGAGAGAGGTCGGTG | |
| | ATTCGGTCAACGAGGGAGCCGACTGCCGACGTGCGCTCCGGA | |
| | GGCTTGCAGAATGCGGAACACCGCGCGGGCAGGAACAGGGCC | |
| | CACACTACCGCCCCACACCCCGCCTCCCGCACCGCCCCTTCCC | |
| | GGCCGCTGCTCTCGGCGCGCCCTGCTGAGCAGCCGCTATTGGC | |
| | CACAGCCCATCGCGGTCGGCGCGCTGCCATTGCTCCCTGGCGC | |
| | TGTCCGTCTGCGAGGGTACTAGTGAGACGTGCGGCTTCCGTTT | |
| | GTCACGTCCGGCACGCCGCGAACCGCAAGGAACCTTCCCGACT | |
| | TAGGGGCGGAGCAGGAAGCGTCGCCGGGGGGCCCACAAGGGT | |
| | AGCGGCGAAGATCCGGGTGACGCTGCGAACGGACGTGAAGAA | |
| | TGTGCGAGACCCAGGGTCGGCGCCGCTGCGTTTCCCGGAACCA | |
| | CGCCCAGAGCAGCCGCGTCCCTGCGCAAACCCAGGGCTGCCTT | |
| | GGAAAAGGCGCAACCCCAACCCCGTGGAAGCTCCAAAGGACT | |
| | CAAAGAACCTCTGGATGCTTTGACATACGATTTTTAATAAAAC | |
| | ATGAGCATTTGAATAAAAACGACTTCCTCATACTGTAAACATC | |
| | ACGCATGCACATTAGACAATAATCCAGTAACGAAACGGCTTCA | |
| | GTCGTAATCGCCCATATAGTTGGCTACAGAATGTTGGATAGAG | |
| | AACTTAAGTACGCTAAGGCGGCGTATTTTCTTAATATTTAGGG | |
| | GTATTGCCGCAGTCATTACAGATACTCAGGAGTCAGATGCACC | |
| | ATGGTGTCTGTTTGAGGTTGCTAGTGAACACAGTTGTGTCAGA | |
| | AGCAAATGTAAGCAATAGATGGCTCTGCCCTGACTTTTATGCC | |
| | CAGCCCTGGCTCCTGCCCTCCCTGCTCCTGGGAGTAGATTGGC | |
| | CAACCCTAGGGTGTGGCTCCACAGGGTGA | |
| Donor template | TACTCTTAATTCATTACATATTGTGCGGTCGAATTCAGGGAGC | 105 |
| design 3 (no HA) | CGATAATGCGGTTACAATAATTCCTATACTTAAATATACAAAG | |
| | ATTTAAAATTTCAAAAAATGGTTACCAGCATCGTTAGTGCGTA | |
| | TACATCAAGAGGCACGTGCCCCGGAGACAGCAAGTAAGCTCT | |
| | TTAAACATGCTTTGACATACGATTTTTAATAAAACATGAGCAT | |
| | TTGAATAAAAACGACTTCCTCATACTGTAAACATCACGCATGC | |
| | ACATTAGACAATAATCCAGTAACGAAACGGCTTCAGTCGTAAT | |
| | CGCCCATATAGTTGGCTACAGAATGTTGGATAGAGAACTTAAG | |
| | TACGCTAAGGCGGCGTATTTTCTTAATATTTAGGGGTATTGCC | |
| | GCAGTCATTACAGATAACCGCCTATGCGGCCATGCCAGGATTA | |
| | TAGATAACTTTTTAACATTAGCCGCAGAGGTGGGACTAGCACG | |
| | TAATATCAGCACATAACGTGTCAGTCAGGTCATCGACCTCGTC | |
| | GGACTCCGGGTGCGAGGTCGTGAAGCTGGAATACGAGTGAGG | |
| | CCGCCGAGGACGTCAGGGGGGTGTAAAGCTTCCATAGAGCCC | |
| | ACCGCATCCCCAGCATGCCTGCTATTGTCTTCCCAATCCTCCCC | |
| | CTTGCTGTCCTGCCCCACCCCACCCCCCAGAATAGAATGACAC | |
| | CTACTCAGACAATGCGATGCAATTTCCTCATTTTATTAGGAAA | |
| | GGACAGTGGGAGTGGCACCTTCCAGGGTCAAGGAAGGCACGG | |
| | GGGAGGGGCAAACAACAGATGGCTGGCAACTAGAAGGCACAG | |
| | TCGAGGCTGATCAGCGGGTTTAAACGGGCCCTCTAGACTCGAC | |
| | GCGGCCGCTTTACTTGTACAGCTCGTCCATGCCGAGAGTGATC | |
| | CCGGCGGCGGTCACGAACTCCAGCAGGACCATGTGATCGCGCT | |
| | TCTCGTTGGGGTCTTTGCTCAGGGCGGACTGGGTGCTCAGGTA | |
| | GTGGTTGTCGGGCAGCAGCACGGGGCCGTCGCCGATGGGGGT | |
| | GTTCTGCTGGTAGTGGTCGGCGAGCTGCACGCTGCCGTCCTCG | |
| | ATGTTGTGGCGGATCTTGAAGTTCACCTTGATGCCGTTCTTCTG | |
| | CTTGTCGGCCATGATATAGACGTTGTGGCTGTTGTAGTTGTACT | |
| | CCAGCTTGTGCCCCAGGATGTTGCCGTCCTCCTTGAAGTCGAT | |
| | GCCCTTCAGCTCGATGCGGTTCACCAGGGTGTCGCCCTCGAAC | |
| | TTCACCTCGGCGCGGGTCTTGTAGTTGCCGTCGTCCTTGAAGA | |
| | AGATGGTGCGCTCCTGGACGTAGCCTTCGGGCATGGCGGACTT | |
| | GAAGAAGTCGTGCTGCTTCATGTGGTCGGGGTAGCGGCTGAAG | |
| | CACTGCACGCCGTAGGTCAGGGTGGTCACGAGGGTGGGCCAG | |
| | GGCACGGGCAGCTTGCCGGTGGTGCAGATGAACTTCAGGGTC | |
| | AGCTTGCCGTAGGTGGCATCGCCCTCGCCCTCGCCGGACACGC | |
| | TGAACTTGTGGCCGTTTACGTCGCCGTCCAGCTCGACCAGGAT | |
| | GGGCACCACCCCGGTGAACAGCTCCTCGCCCTTGCTCACCATG | |
| | GTGGCGACCGGTGGGGAGAGAGGTCGGTGATTCGGTCAACGA | |
| | GGGAGCCGACTGCCGACGTGCGCTCCGGAGGCTTGCAGAATG | |
| | CGGAACACCGCGCGGGCAGGAACAGGGCCCACACTACCGCCC | |
| | CACACCCCGCCTCCCGCACCGCCCCTTCCCGGCCGCTGCTCTC | |
| | GGCGCGCCCTGCTGAGCAGCCGCTATTGGCCACAGCCCATCGC | |
| | GGTCGGCGCGCTGCCATTGCTCCCTGGCGCTGTCCGTCTGCGA | |
| | GGGTACTAGTGAGACGTGCGGCTTCCGTTTGTCACGTCCGGCA | |
| | CGCCGCGAACCGCAAGGAACCTTCCCGACTTAGGGGCGGAGC | |
| | AGGAAGCGTCGCCGGGGGGCCCACAAGGGTAGCGGCGAAGAT | |
| | CCGGGTGACGCTGCGAACGGACGTGAAGAATGTGCGAGACCC | |
| | AGGGTCGGCGCCGCTGCGTTTCCCGGAACCACGCCCAGAGCA | |
| | GCCGCGTCCCTGCGCAAACCCAGGGCTGCCTTGGAAAAGGCG | |
| | CAACCCCAACCCCGTGGAAGCTTGCGACCTGGAATCGGACAG | |
| | CAGCGGGGAGTGTACGGCCCCGAGTTCGTGACCGGGTATGCTT | |
| | TCATTGTACCCCGGAACTTTAAATCTATGAACAATCGCAACAA | |
| | ATTGTCCAAAGGCAACAATACGACACAGTTAGAGGCCATCGG | |
| | CGCAGGTACACTCTATCCACGCCTATCAGAATGTCACCTGGTT | |
| | AATGGTCAATTTAGGTGGCTGGAGGCACATGTGAAGCAATATG | |
| | GTCTAGGGAAAGATATCGGTTTACTTAGATTTTATAGTTCCGG | |
| | ATCCAACTTAAATAATATAGGTATTAAAGAGCAGTATCAAGAG | |
| | GGTTTCTTCCCAAGGAATCTTGCGATTTTCATACACAGCTTTAA | |
| | CAAATTTCACTAGACGCACCTTCATTTTGTCGTCTCGTTGTATA | |
| | TGAGTCCGGGGTAAGAATTTTTTACCGTATTTAACATGATCAA | |
| | CGGGTACTAAAGCAATGTCATTTCTAAACACAGTAGGTAAAGG | |
| | ACACGTCATCTTATTTTAAAGAATGTCAGAAATCAGGGAGACT | |
| | AGATCGATATTACGTGTTTT | |

Targeted integration experiments were conducted in primary CD4+ T cells with wild-type S. pyogenes ribonucleoprotein (RNP) targeted to the HBB locus. AAV6 was added at different multiplicities of infection (MOI) after nucleofection of 50 pmol of RNP. GFP fluorescence was measured 7 days after the experiment and showed that targeted integration frequency with the shorter homology arms was as efficient as when the longer homology arms were used (FIG. 2B). Assessment of targeted integration by digital droplet PCR (ddPCR) to either the 5′ or 3′ integration junction showed that (1) HA length did not affect targeted integration and (2) phenotypic assessment of targeted integration by GFP expression dramatically underestimated actual genomic targeted integration.

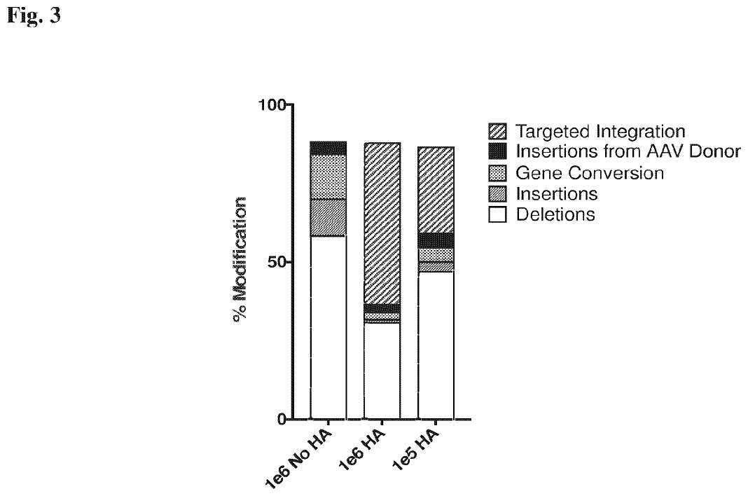

The genomic DNA from the cells that received the 177 nt HA donor (1e6 or 1e5 MOI) or no HA donor (1e6 MOI) was amplified with the 5′ and 3′ primers (P1 and P2), the PCR fragment was subcloned into a Topo Blunt Vector, and the resulting plasmids were Sanger sequenced. All high quality reads mapped one of the three expected PCR amplicons and the total number of reads were: 1e6 No HA—77 reads, 1e6 HA Donor—422 reads, 1e5 HA Donor—332 reads. The analysis allowed for the determination of on-target editing events at the HBB locus, including insertions, deletions, gene conversion from the highly homologous HBD gene, insertions from fragmented AAV donors, and targeted integration (FIG. 3A). To calculate targeted integration, the following formulas were used, taking into account the total number of reads from the 1^{st} Amplicon (AmpX), 2^{nd} Amplicon (AmpY), and 3^{rd} Amplicon (AmpZ). The results are summarized in Table 8 below.

$$\\ Sequencing ( Overall ) = \frac{Average ( AmpY + AmpZ )}{AmpX + Average ( AmpY + AmpZ )} \times 100$$

$$\\ Sequencing ( {{5}^{}}^{′} ) = \frac{AmpY}{AmpX + AmpY} \times 100$$

$$\\ Sequencing ( {{3}^{}}^{′} ) = \frac{AmpZ}{AmpX + AmpZ} \times 100$$

TABLE 8
Comparison of Targeted Integration
Frequency at HBB locus Using
Different Methods of Calculation.

| | | % |
|---|---|---|
| | Assay | Integration |
| 1e6 MOI | GFP | 9.6% |
| | 5' ddPCR | 70% |
| | 3' ddPCR | 62% |
| | Sequencing | 51% |
| | (Overall) | |
| | Sequencing | 57% |
| | (5' Junction) | |
| | Sequencing | 43.9% |
| | (3' Junction) | |
| 1e5 MOI | GFP | 4.3% |
| | 5' ddPCR | 21.9% |
| | 3' ddPCR | 20% |
| | Sequencing | 27.2% |
| | (Overall) | |
| | Sequencing | 31.9% |
| | (5' Junction) | |
| | Sequencing | 21.8% |
| | (3' Junction) | |

The sequencing (overall) formula described above provided an estimate for the targeted integration taking into consideration reads from both the 2^{nd} amplicon (AmpY) and 3^{rd} amplicon (AmpZ). When either the 2^{nd} amplicon (AmpY) or 3^{rd} amplicon (AmpZ) was used alone to calculate targeted integration, the output was similar, showing that this method can be used with only 1 integrated priming site (either P1′ or P2′). The sequencing read-out matched the ddPCR analysis from either the 5′ or 3′ junction, indicating no PCR biases in the amplification, and that this method can be used to determine all on-target editing events.

### Example 2: Targeted Integration at the HBB Locus in Adult Mobilized Peripheral Blood Human CD34⁺ Cells

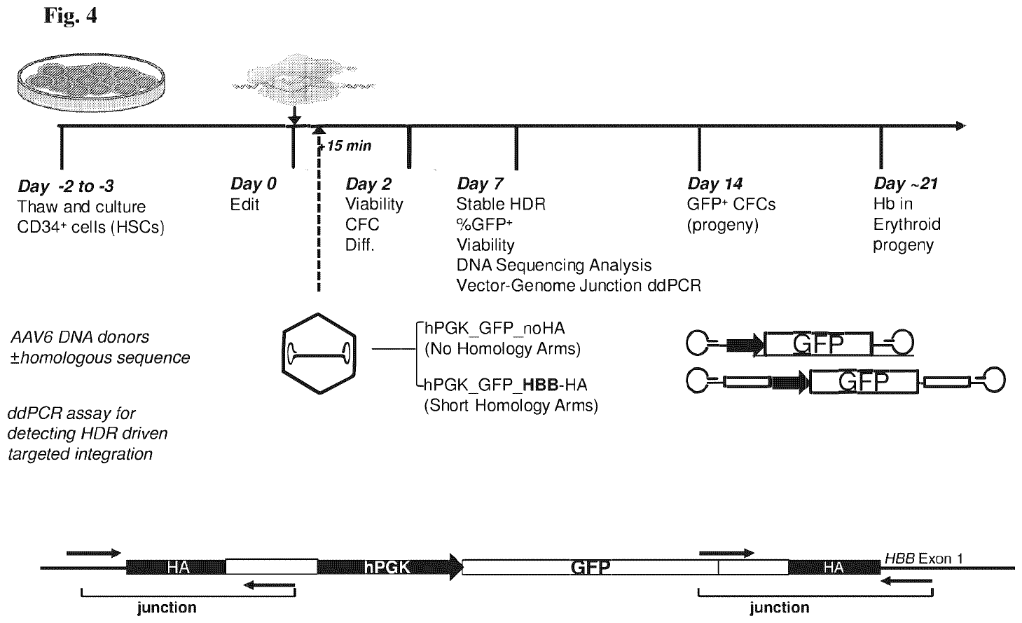

In order to determine the baseline level of targeted integration at the HBB locus in hematopoietic stem/progenitor cells, the population of cells which would be targeted clinically for gene correction or cDNA replacement for the treatment of b-hemoglobinopathies, the donor as described in Example 1 and depicted in FIG. 2A and Table 7, were used to deliver the PGK-GFP transgene expression cassette flanked by short homology arms (HA). The experimental schematic, timing and readouts for targeted integration are depicted in FIG. 4. Targeted integration experiments were conducted in human mobilized peripheral blood (mPB) CD34⁺ cells with wild-type S. pyogenes ribonucleoprotein (RNP) targeted to the HBB locus. Cells were cultured for 3 days in StemSpan-SFEM supplemented with human cytokines (SCF, TPO, FL, IL6) and dmPGE2. Cells were electroporated with the Maxcyte System and AAV6±HA (vector dose: 5×10⁴ vg/cell) was added to the cells 15-30 minutes after electroporation of the cells with UM RNP (CAGACUUCUCCACAGGAGUC (SEQ ID NO: 118)). Two days after electroporation, CD34+ cell viability was assessed, and cells were plated into Methocult to evaluate ex vivo hematopoietic differentiation potential and expression of GFP in their erythroid and myeloid progeny. On day 7 after electroporation, GFP fluorescence was evaluated by flow cytometry analysis in the viable CD34+ cell fraction. In addition, assessment of targeted integration was also analyzed by digital droplet PCR (ddPCR) to both the 5′ or 3′ integration junction. ddPCR analysis and Sanger sequencing analysis were done as described in Example 1.

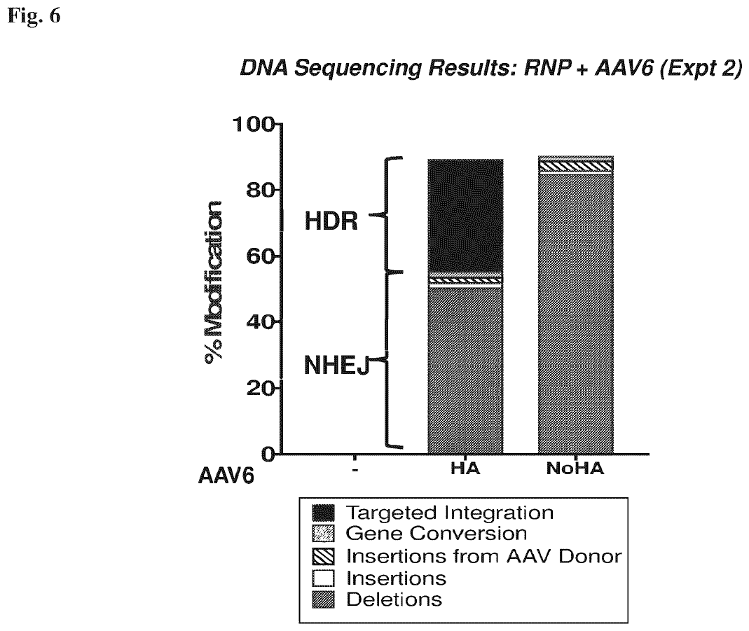

Three separate experiments were conducted and the day 7 targeted integration results are depicted in FIG. 5. Targeted integration as determined by 5′ and 3′ ddPCR analysis was ˜35% (FIG. 5A, 5B). Expression of the integration GFP transgene in CD34⁺ cells 7 days after electroporation was consistent with the ddPCR data, indicating that the integrated transgene was expressed. DNA sequencing analysis confirmed these results, with 35% HDR and 55% NHEJ detected in gDNA of CD34+ cells treated with RNP and AAV6 with HA (FIG. 6, total editing 90%). In contrast, CD34+ cells treated with RNP and AAV6 without HA, no targeted integration was detected, the only HDR observed was 1.7% gene conversion (that is gene conversion between HBB and HBD), while total editing frequency was the same (90%).

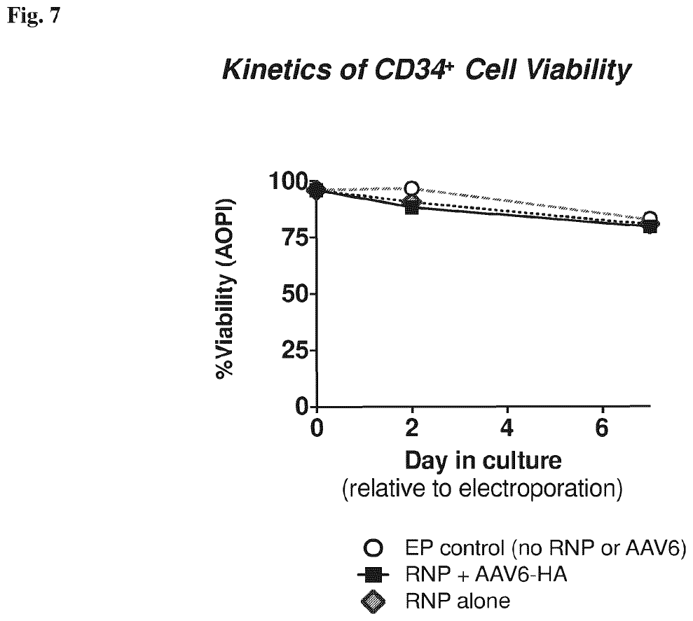

Importantly, between days 0 and 7 after electroporation there was no substantial difference in the viability (as determined by AOPI) of cells treated with RNP+AAV or untreated (EP electroporation control) (FIG. 7). This suggests that the RNP and AAV6 combination is well-tolerated by CD34⁺ cells.

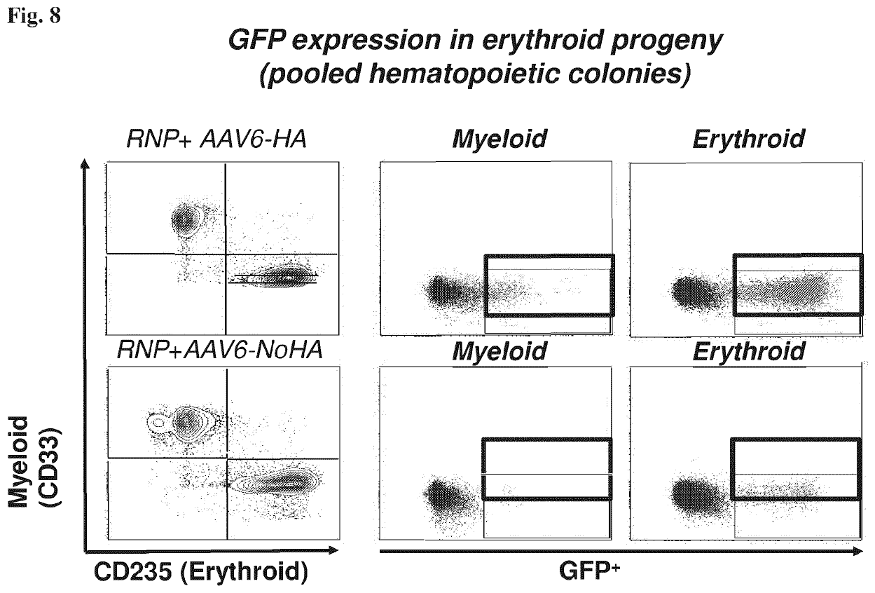

To determine whether the cells containing the targeted integration maintain differentiation potential, CD34⁺ cells on day 2 were plated into Methocult to evaluate ex vivo hematopoietic activity. On day 14 after plating CD34⁺ cells into Methocult, GFP⁺ colonies were scored by fluorescence microscopy For the CD34⁺ cells treated with RNP with AAV6-HA and RNP with AAV6 with no HA the percentages of GFP⁺ colonies were 32% and 2%, respectively. Pooled colonies were collected, pooled, immunostained with anti-human CD235 antibody (detecting Glycophorin A, erythroid specific cell surface antigen) and anti-human CD33 antibody (detected a myeloid specific cell surface antigen) and the analyzed by flow cytometry analysis. GFP expression was higher in the CD235⁺ erythroid vs. CD33⁺ myeloid cell fraction for progeny of cells treated with AAV6 (FIG. 8). This suggests that although the human PGK promoter is regulating transgene expression, higher expression occurs in the erythroid progeny, consistent with the integration of this gene into erythroid specific location (HBB gene). These data also show that integration is maintained in differentiated progeny of HDR-edited CD34⁺ cells.

### Example 3: Detection of Targeted Integration at the TRAC Locus

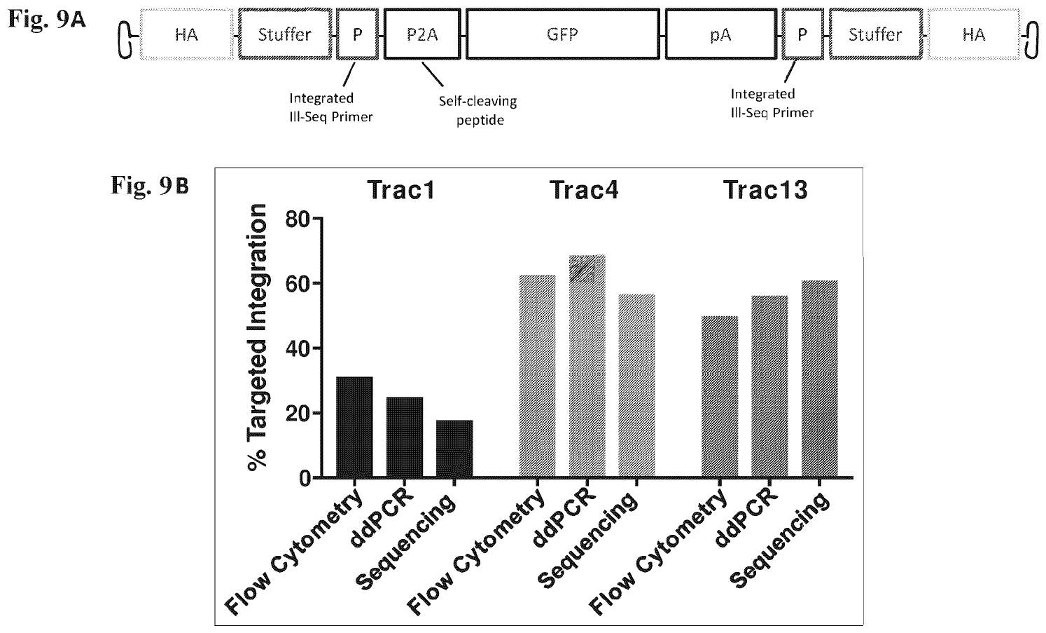

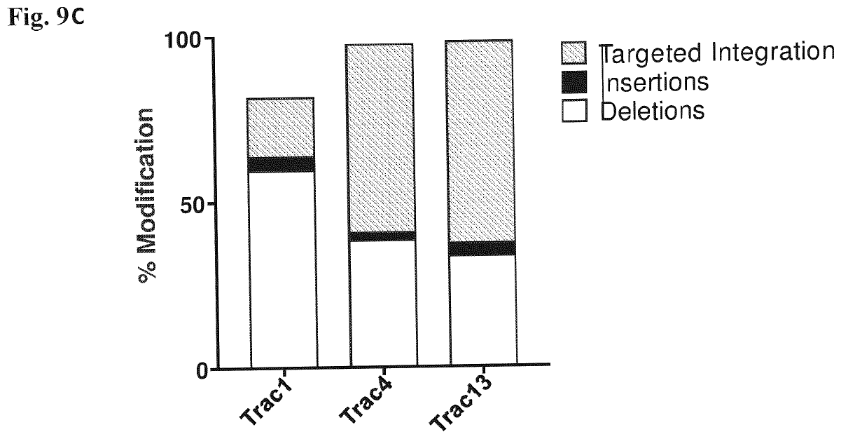

Exemplary DNA donor templates were designed for use with three different gRNAs targeting the T cell receptor alpha constant (TRAC) locus (TRAC1, TRAC4, and TRAC13), as shown in FIG. 9A. Each donor contained the same cargo (P2A-GFP-polyA sequence), but with different homology arm sequences designed to start immediately 5′ and 3′ of the predicted cut site. The integrated primer sequences were unique to the donors, and the homology arms extend from the cut site to the beginning of the primer binding site in the genome. The homology arm length used for each donor is provided in Table 9. Primer sequences and homology arm sequences for each donor are provided in Table 10. Targeted integration experiments were conducted in primary CD4+ T cells using wild-type S. pyogenes ribonucleoprotein with the appropriate gRNA and associated donor template. Cells were expanded after the experiment until Day 7, when flow cytometry was conducted to check the rate of targeted integration by GFP expression. The P2A self-cleaving peptide must be joined in-frame with the endogenous TRAC locus to obtain expression of GFP. Since the donor templates each contain a P2A-GFP cassette, GFP is not expressed from the donor template alone, as the P2A-GFP cassette requires a promoter to initiate expression and also must be in frame with the preceding transcription. Accordingly, only accurate HDR-driven targeted integration results in GFP expression.

After flow cytometry was conducted, cells were collected, gDNA was isolated, and targeted integration was assessed by ddPCR and sequencing of amplicons generated from amplification of the 5′ and 3′ junctions of the targeted integration event using the integrated priming sites with 5′ and 3′ primers (P1 and P2), as described herein. The results from the three analyses (fluorescent flow cytometry, ddPCR, and sequencing) at the three gRNA sites is shown in FIG. 9B. Importantly, all three methods of analysis showed consistent results. Sequencing using the integrated priming sites advantageously provides a complete picture of all editing events at the cut site, as shown in FIG. 9C. This figure depicts all editing events that took place during targeted integration experiments after cuts were introduced using TRAC1, TRAC4, and TRAC13, including insertions, deletions and targeted integration. This analysis was completed by using the integrated primers to amplify gDNA, followed by Sanger sequencing and alignment to one of the three expected amplicons.

TABLE 9
Homology Arm Length
in donor templates for
targeted integration at
the TRAC locus

| | 5' HA | 3' HA |
|---|---|---|
| | Length | Length |
| TRAC1 | 281 bp | 140 bp |
| TRAC4 | 142 bp | 311 bp |
| TRAC13 | 188 bp | 259 bp |

TABLE 10
Primer Sequences and Homology Arm Sequences for TRAC donor templates

| | Primer Sequences | HA Sequences |
|---|---|---|
| TRAC1 | P1 (SEQ ID NO: 106): | 5′ (SEQ ID NO: 108): |
| | GCATTTCAGGTTTCCTT | CAGGCCAGGCCTGGCGTGAACGTTCACTGAAAT |
| | GAGTGG | CATGGCCTCTTGGCCAAGATTGATAGCTTGTGCC |
| | P2 (SEQ ID NO: 107): | TGTCCCTGAGTCCCAGTCCATCACGAGCAGCTGG |
| | GCACTGTTGCTCTTGAA | TTTCTAAGATGCTATTTCCCGTATAAAGCATGAG |
| | GTCC | ACCGTGACTTGCCAGCCCCACAGAGCCCCGCCC |
| | | TTGTCCATCACTGGCATCTGGACTCCAGCCTGGG |
| | | TTGGGGCAAAGAGGGAAATGAGATCATGTCCTA |
| | | ACCCTGATCCTCTTGTCCCACAGATATCCAGAAC |
| | | CCTGACCCTGCC |
| | | 3′ (SEQ ID NO: 109): |
| | | GTGTACCAGCTGAGAGACTCTAAATCCAGTGAC |
| | | AAGTCTGTCTGCCTATTCACCGATTTTGATTCTC |
| | | AAACAAATGTGTCACAAAGTAAGGATTCTGATG |
| | | TGTATATCACAGACAAAACTGTGCTAGACATGA |
| | | GGTCTAT |
| TRAC4 | P1 (SEQ ID NO: 110): | 5′ (SEQ ID NO: 112): |
| | CTTGTCCATCACTGGCA | ACTCCAGCCTGGGTTGGGGCAAAGAGGGAAATG |
| | TCTGG | AGATCATGTCCTAACCCTGATCCTCTTGTCCCAC |
| | P2 (SEQ ID NO: 111): | AGATATCCAGAACCCTGACCCTGCCGTGTACCA |
| | TTTGGTGGCAATGGATA | GCTGAGAGACTCTAAATCCAGTGACAAGTCTGT |
| | AGGC | CTGCCTATT |
| | | 3′ (SEQ ID NO: 113): |
| | | CACCGATTTTGATTCTCAAACAAATGTGTCACAA |
| | | AGTAAGGATTCTGATGTGTATATCACAGACAAA |
| | | ACTGTGCTAGACATGAGGTCTATGGACTTCAAG |
| | | AGCAACAGTGCTGTGGCCTGGAGCAACAAATCT |
| | | GACTTTGCATGTGCAAACGCCTTCAACAACAGC |
| | | ATTATTCCAGAAGACACCTTCTTCCCCAGCCCAG |
| | | GTAAGGGCAGCTTTGGTGCCTTCGCAGGCTGTTT |
| | | CCTTGCTTCAGGAATGGCCAGGTTCTGCCCAGAG |
| | | CTCTGGTCAATGATGTCTAAAACTCCTCTGATTG |
| | | GTGGTCTCG |
| TRAC13 | P1 (SEQ ID NO: 114): | 5′ (SEQ ID NO: 116): |
| | TCCTAACCCTGATCCTC | CACAGATATCCAGAACCCTGACCCTGCCGTGTA |
| | TTGTCC | CCAGCTGAGAGACTCTAAATCCAGTGACAAGTC |
| | P2 (SEQ ID NO: 115): | TGTCTGCCTATTCACCGATTTTGATTCTCAAACA |
| | CCGTGTCATTCTCTGGA | AATGTGTCACAAAGTAAGGATTCTGATGTGTAT |
| | CTGC | ATCACAGACAAAACTGTGCTAGACATGAGGTCT |
| | | ATGGACTTCAAGAGCAACAGTG |
| | | 3′ (SEQ ID NO: 117): |
| | | CTGTGGCCTGGAGCAACAAATCTGACTTTGCATG |
| | | TGCAAACGCCTTCAACAACAGCATTATTCCAGA |
| | | AGACACCTTCTTCCCCAGCCCAGGTAAGGGCAG |
| | | CTTTGGTGCCTTCGCAGGCTGTTTCCTTGCTTCA |
| | | GGAATGGCCAGGTTCTGCCCAGAGCTCTGGTCA |
| | | ATGATGTCTAAAACTCCTCTGATTGGTGGTCTCG |
| | | GCCTTATCCATTGCCACCAAAACCCTCTTTTTAC |
| | | TAAGAAACAGTGAGCCTTGTTCTG |

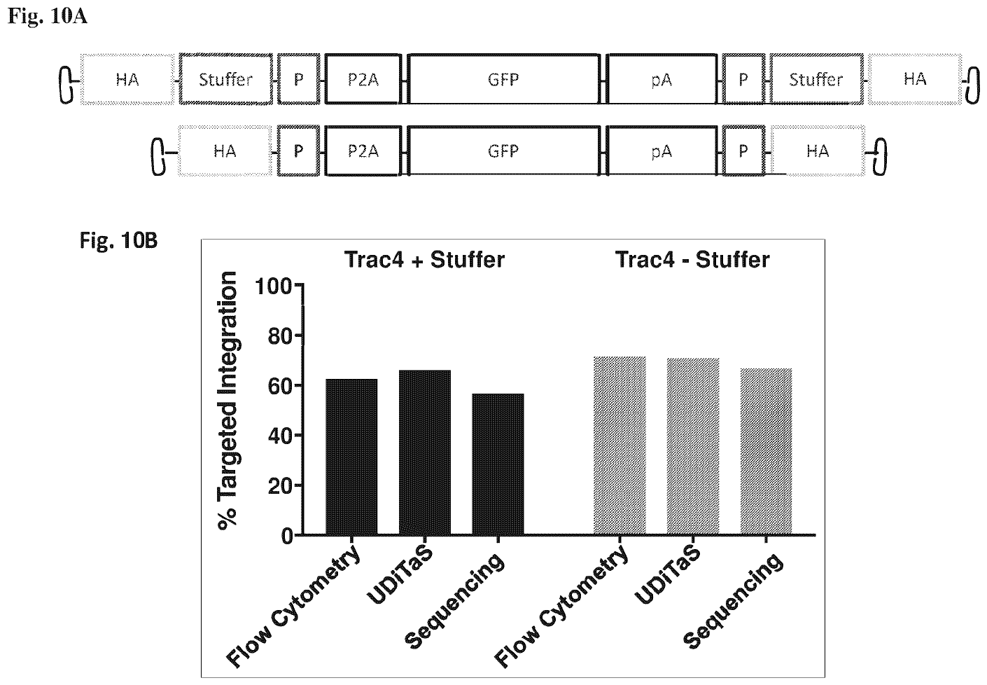

DNA donor templates for targeted integration at the TRAC4 cut site were designed with and without stuffer sequence, as shown in FIG. 10A. Targeted integration experiments were conducted in primary CD4+ T cells using wild-type S. pyogenes ribonucleoprotein with the TRAC4 gRNA and either AAV-donor. Cells were expanded after the experiment until Day 7 when flow cytometry was conducted to check the rate of targeted integration by GFP expression. After flow cytometry was conducted, cells were collected, gDNA was isolated, and targeted integration was assessed by sequencing using the integrated priming sites, as described above, and UDITAS™, a unidirectional sequencing method described herein. The results from all three methods of assessing targeted integration efficiency are shown in FIG. 10B. The three methods of assessing targeted integration efficiency demonstrated consistent results, with or without the use of stuffer sequence in the donor template, indicating that targeted integration is not dependent on the presence of the stuffer sequence.

Additional donor templates for targeted integration at the TRAC locus were prepared as described in Table 11. Briefly, donors contain long or short homology arms (HA) flaking the cut site of gRNAs TRAC1, TRAC4, or TRAC13. Constructs containing short homology arms additionally contain primer sequences P1 and P2 flanking the cargo, as described above (see Table 9 and Table 10). Constructs were prepared with and without stuffer sequence, and with P2A or Promoter cargo, as indicated. Additional donor templates lacking homology arms were prepared as a negative control.

TABLE 11
Donors for targeted integration at the TRAC locus

| | gRNA | HA Length | Stuffer | Cargo |
|---|---|---|---|---|
| 1. | TRAC-1 | Short | Yes | P2A + GFP |
| 2. | TRAC-4 | Short | Yes | Promoter + GFP |
| 3. | TRAC-4 | Short | Yes | P2A + GFP |
| 4. | TRAC-4 | Short | No | P2A + GFP |
| 5. | TRAC-4 | Long (500 bp) | No | P2A + GFP |
| 6. | TRAC-13 | Short | Yes | P2A + GFP |
| 7. | No HA | n/a | N/A | P2A + GFP |
| 8. | No HA | n/a | N/A | PGK + GFP |

Targeted integration experiments were conducted in primary CD4+ T cells using wild-type S. pyogenes ribonucleoprotein with TRAC1, TRAC4, or TRAC13 gRNA, and the appropriate AAV-donor as shown in Table 11.

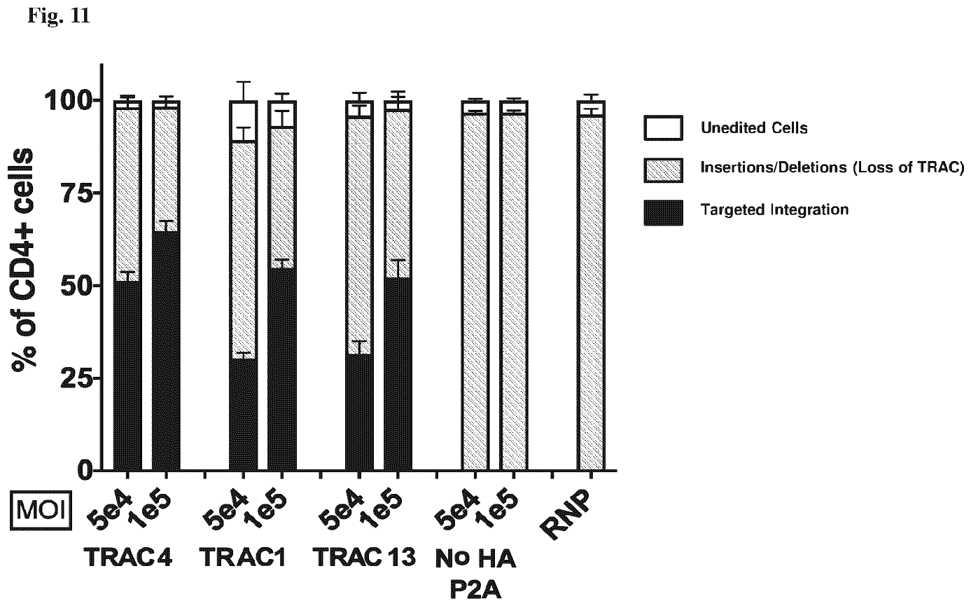

Cells were expanded after the experiment until Day 7 when flow cytometry was conducted to check the rate of targeted integration by GFP expression. Editing outcomes were assessed using flow cytometry. Targeted integration efficiency at the TRAC locus is shown in FIG. 11 (from left to right, Donor 3, Donor 1, Donor 6, Donor 7; RNP=without donor). The rate of each editing outcome was validated by sequencing amplification products generated using primers P1 and P2, as described herein, and by ddPCR, as shown in Table 12. A high rate of targeted integration was observed at three gRNA cut sites in Exon 1 of TRAC, using comparable donors at two concentrations. Rates of targeted integration are shown in Table 12. Levels of targeted integration at the TRAC1 site were reduced slightly due to contamination with TRAC13.

TABLE 12
Targeted integration
frequency at three gRNA
sites in Exon 1 of TRAC
using comparable donors

| | | | Sequencing |
|---|---|---|---|
| | | | Amplification |
| | Flow | | Products from |
| | Cytometry | | Integrated |
| | (GFP) | ddPCR | Priming sites |
| Trac1 | 28% | 24% | 18% |
| Trac4 | 61% | 67% | 57% |
| Trac13 | 49% | 56% | 60% |

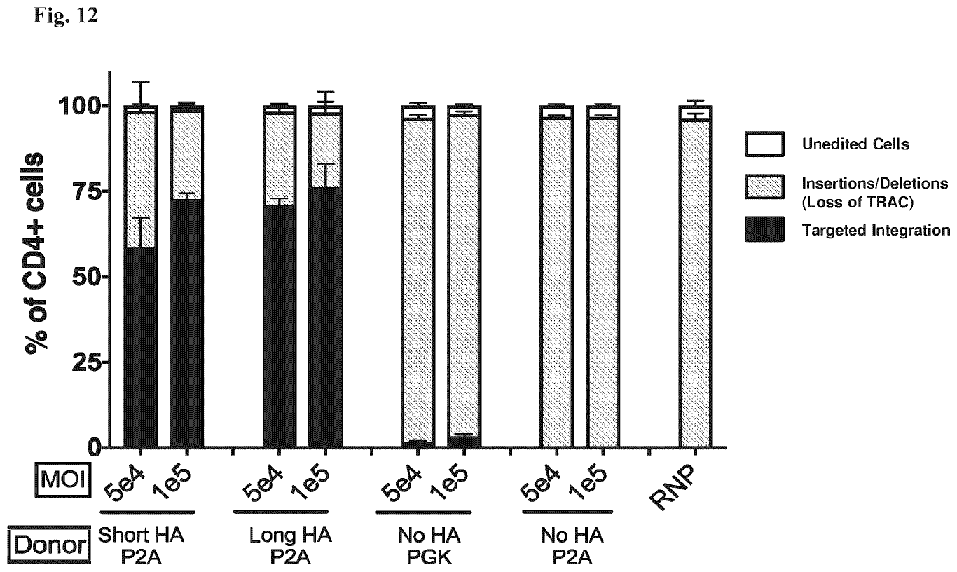

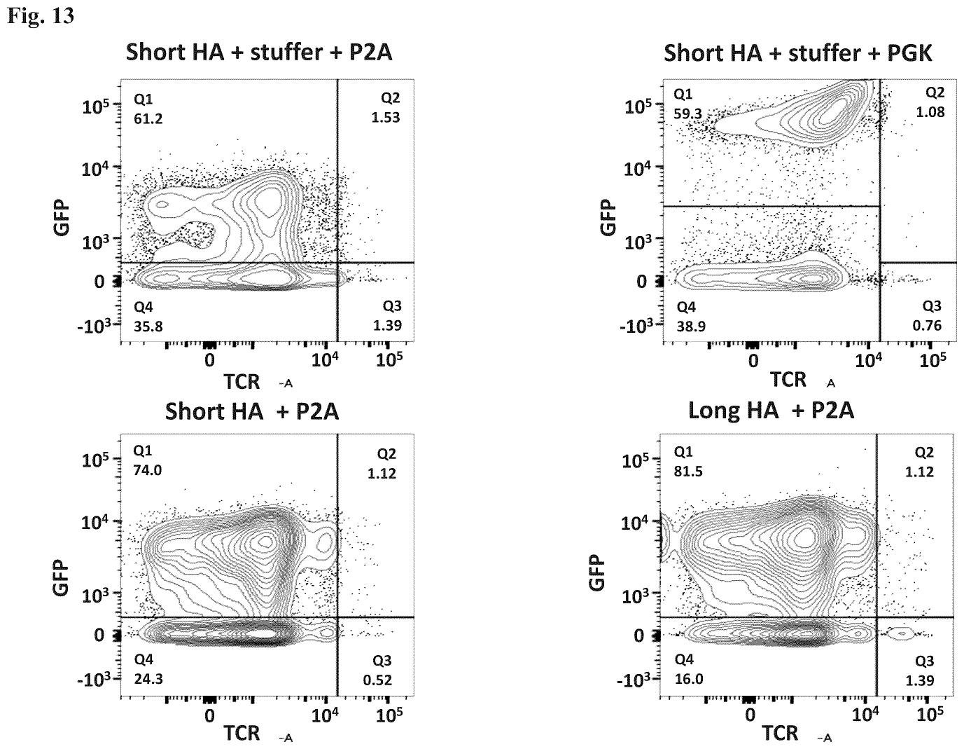

The foregoing experiment was repeated with multiple donors targeting the TRAC4 locus. Results are shown in FIG. 12 (from left to right, Donor 4, Donor 5, Donor 8, Donor 7, RNP only (no donor). Flow cytometry measuring GFP and TRAC expression in edited cells is shown in FIG. 13. Consistently high rates of targeted integration were observed at the TRAC4 locus, across multiple AAV donors, preparations, and MOIs tested. Donors containing long homology arms (500 bp) had higher levels of targeted integration than donors containing shorter homology arms.

### INCORPORATION BY REFERENCE

All publications, patents, and patent applications mentioned herein are hereby incorporated by reference in their entirety as if each individual publication, patent or patent application was specifically and individually indicated to be incorporated by reference. In case of conflict, the present application, including any definitions herein, will control.

### EQUIVALENTS

Those skilled in the art will recognize, or be able to ascertain using no more than routine experimentation, many equivalents to the specific embodiments described herein. Such equivalents are intended to be encompassed by the following claims.

1. An isolated nucleic acid for homologous recombination with a target nucleic acid having a cleavage site, wherein:
(a) a first strand of the target nucleic acid comprises, from 5′ to 3′, P1--H1--X--H2--P2, wherein
P1 is a first priming site;
H1 is a first homology arm;
X is the cleavage site;
H2 is a second homology arm; and
P2 is a second priming site; and
(b) a first strand of the isolated nucleic acid comprises, from 5′ to 3′, A1--P2′--N--A2, or A1--N--P1′--A2, wherein
A1 is a homology arm that is substantially identical to H1;
P2′ is a priming site that is substantially identical to P2;
N is a cargo;
P1′ is a priming site that is substantially identical to P1; and
A2 is a homology arm that is substantially identical to H2.

2. The isolated nucleic acid of claim 1, wherein the first strand of the isolated nucleic acid comprises, from 5′ to 3′, A1--P2′--N--P1′--A2.

3. The isolated nucleic acid of claim 1, further comprising S1 or S2, wherein the first strand of the isolated nucleic acid comprises, from 5′ to 3′, A1--S1--P2′--N--A2, or A1--N--P1′--S2--A2;
wherein S1 is a first stuffer, wherein S2 is a second stuffer, and wherein each of S1 and S2 comprise a random or heterologous sequence having a GC content of approximately 40%.

4. The isolated nucleic acid of claim 3, wherein:
(i) the first stuffer has a sequence having less than 50% sequence identity to any nucleic acid sequence within 500 base pairs of the cleavage site, and wherein the second stuffer has a sequence having less than 50% sequence identity to any nucleic acid sequence within 500 base pairs of the cleavage site;
(ii) the first stuffer has a sequence comprising at least 10 nucleotides of a sequence set forth in Table 2, and wherein the second stuffer has a sequence comprising at least 10 nucleotides of a sequence set forth in Table 2;
(iii) the first stuffer has a sequence that is not the same as the sequence of the second stuffer; or
(iv) the first strand of the isolated nucleic acid comprises, from 5′ to 3′, A1--S1--P2′--N--P1′--S2--A2.

5.-7. (canceled)

8. The isolated nucleic acid of claim 1, wherein:
(i) A1 has a sequence that is at least 40 nucleotides in length, and A2 has a sequence that is at least 40 nucleotides in length;
(ii) A1 has a sequence that is identical to, or differs by no more than 1, 2, 3, 4, 5, 6, 7, 8, 9, 10, 15, 20, 25, or 30 nucleotides from a sequence of H1;
(iii) A2 has a sequence that is identical to, or differs by no more than 1, 2, 3, 4, 5, 6, 7, 8, 9, 10, 15, 20, 25, or 30 nucleotides from a sequence of H2;
(iv) N comprises an exon of a gene sequence, an intron of a gene sequence, a cDNA sequence, or a transcriptional regulatory element; a reverse complement of any of the foregoing or a portion of any of the foregoing; or
(v) N comprises a promoter sequence.

9.-12. (canceled)

13. A composition comprising the isolated nucleic acid of claim 1 and a pharmaceutically acceptable carrier.

14. A vector comprising the isolated nucleic acid of claim 1.

15.-16. (canceled)

17. A genome editing system comprising the isolated nucleic acid of claim 1.

18.-68. (canceled)

69. A method for determining the outcome of a gene editing event at a cleavage site in a target nucleic acid in a cell using an exogenous donor template,
wherein the target nucleic acid comprises a first strand comprising: a first homology arm 5′ to a cleavage site, a first priming site either within the first homology arm or 5′ to the first homology arm, a second homology arm 3′ to the cleavage site, and a second priming site either within the second homology arm or 3′ to the second homology arm, and
wherein a first strand of the exogenous donor template comprises i) a cargo, a priming site that is substantially identical to the second priming site either within or 5′ to the cargo, a first donor homology arm 5′ to the cargo, and a second donor homology arm 3′ to the cargo; or ii) a cargo, a first donor homology arm 5′ to the cargo, a priming site that is substantially identical to the first priming site 3′ to the cargo, and a second donor homology arm 3′ to the cargo,
the method comprising:
i) forming at least one single- or double-strand break at or near the cleavage site in the target nucleic acid;
ii) recombining the exogenous oligonucleotide donor template with the target nucleic acid via homologous recombination to produce an altered nucleic acid; and
iii) amplifying the altered nucleic acid using a first primer which binds to the first priming site and/or the priming site that is substantially identical to the first priming site; and/or a second primer which binds to the second priming site and/or the priming site that is substantially identical to the second priming site;
thereby determining the outcome of the gene editing event in the cell.

70. The method of claim 69, wherein the step of forming the at least one single- or double-strand break comprises contacting the cell with an RNA-guided nuclease.

71.-72. (canceled)

73. The method of claim 69, wherein the first strand of the exogenous oligonucleotide donor template comprises, from 5′ to 3′, the first donor homology arm, the priming site that is substantially identical to the second priming site, the cargo, the priming site that is substantially identical to the first priming site, and the second donor homology arm.

74. The method of claim 69, wherein the first strand of the exogenous oligonucleotide donor template further comprises a first stuffer and/or a second stuffer,
wherein the first stuffer and the second stuffer each comprise a random or heterologous sequence having a GC content of approximately 40%; and
wherein the exogenous oligonucleotide donor template comprises, from 5′ to 3′,
i) the first donor homology arm, the first stuffer, the priming site that is substantially identical to the second priming site, and the second donor homology arm; or
ii) the first donor homology arm, the cargo, the priming site that is substantially identical to the first priming site, the second stuffer, and the second donor homology arm.

75. The method of claim 74, wherein:
(i) the first stuffer has a sequence having less than 50% sequence identity to any nucleic acid sequence within 500 base pairs of the cleavage site, and wherein the second stuffer has a sequence having less than 50% sequence identity to any nucleic acid sequence within 500 base pairs of the cleavage site;
(ii) the first stuffer has a sequence comprising at least 10 nucleotides of a sequence set forth in Table 2, and wherein the second stuffer has a sequence comprising at least 10 nucleotides of a sequence set forth in Table 2;
(iii) the first stuffer has a sequence that is not the same as the sequence of the second stuffer;
(iv) the first strand of the exogenous oligonucleotide donor template comprises, from 5′ to 3′, the first donor homology arm, the first suffer, the priming site that is substantially identical to the second priming site, the cargo, the priming site that is substantially identical to the first priming site, the second stuffer, and the second donor homology arm;
(v) when the altered nucleic acid comprises a targeted integration genome editing event at the cleavage site, amplifying the altered nucleic acid using the first primer and the second primer produces a first amplicon, wherein the first amplicon has a sequence that is substantially identical to a sequence consisting of either (a) the first donor homology arm and the first stuffer, or (b) the second stuffer and the second donor homology arm;
(vi) the cell is a population of cells, and wherein, when the altered nucleic acid in all the cells within the population of cells comprises a targeted integration genome editing event at the cleavage site, amplifying the altered nucleic acid using the first primer and the second primer produces a first amplicon, wherein the first amplicon has a sequence that is substantially identical to a sequence consisting of either i) the first donor homology arm and the first stuffer, or ii) the second stuffer and the second donor homology arm; or
(vii) when the cell is a population of cells, wherein, when the altered nucleic acid in a first cell within the population of cells comprises a non-targeted integration genome editing event at the cleavage site, amplifying the altered nucleic acid using the first primer and the second primer produces a first amplicon, wherein the first amplicon has a sequence that comprises an indel as compared to a sequence of the target nucleic acid; and
wherein, when the altered nucleic acid in a second cell within the population of cells comprises a targeted integration genome editing event at the cleavage site, amplifying the altered nucleic acid in the second cell using the first primer and the second primer produces a second amplicon, wherein the second amplicon has a sequence that is substantially identical to a sequence consisting of either (a) the first donor homology arm and the first stuffer, or (b) the second stuffer and the second donor homology arm.

76.-78. (canceled)

79. The method of claim 69, wherein the altered nucleic acid comprises, from 5′ to 3′,
i) the first priming site, the first donor homology arm, the priming site that is substantially identical to the second priming site, the cargo, the second donor homology arm, and the second priming site; or
ii) the first priming site, the first donor homology arm, the cargo, the priming site that is substantially identical to the first priming site, the second donor homology arm, and the second priming site.

80.-81. (canceled)

82. The method of claim 69, wherein, when the altered nucleic acid comprises a non-targeted integration genome editing event at the cleavage site, amplifying the altered nucleic acid using the first primer and the second primer produces a first amplicon, wherein the first amplicon has a sequence that comprises an indel as compared to a sequence of the target nucleic acid.

83.-84. (canceled)

85. The method of claim 69, wherein the cell is a population of cells, and wherein, when the altered nucleic acid in all cells within the population of cells comprises a non-targeted integration genome editing event at the cleavage site, amplifying the altered nucleic acid using the first primer and the second primer produces a first amplicon, wherein the first amplicon has a sequence that comprises an indel as compared to a sequence of the target nucleic acid.

86.-89. (canceled)

90. A cell, or a population of cells, altered by the method of claim 69.

91. A cell comprising an altered nucleic acid, wherein a first strand of the altered nucleic acid comprises, from 5′ to 3′,
(i) a first priming site, a first donor homology arm, a cargo, a priming site that is substantially identical to the first priming site, a second donor homology arm, and a second priming site;
(ii) a first priming site, a first donor homology arm, a priming site that is substantially identical to a second priming site, a cargo, a second donor homology arm, and the second priming site; or
(iii) a first priming site, a first donor homology arm, a priming site that is substantially identical to a second priming site, a cargo, a priming site that is substantially identical to the first priming site, a second donor homology arm, and the second priming site.

92. The cell of claim 91, wherein the altered nucleic acid further comprises a first stuffer or a second stuffer, wherein the first strand of the altered nucleic acid comprises, from 5′ to 3′,
(i) a first priming site, a first donor homology arm, a cargo, a priming site that is substantially identical to the first priming site, a first stuffer, a second donor homology arm, and a second priming site;
(ii) a first priming site, a first donor homology arm, a first stuffer, a priming site that is substantially identical to a second priming site, a cargo, a second donor homology arm, and the second priming site; or
(iii) a first priming site, a first donor homology arm, a first stuffer, a priming site that is substantially identical to a second priming site, a cargo, a priming site that is substantially identical to the first priming site, a second stuffer, a second donor homology arm, and the second priming site.

93. The cell of claim 91, wherein the cell is a eukaryotic cell.

94.-104. (canceled)

105. The cell of claim 91, wherein the cell is derived from a cell comprising an unaltered nucleic acid, wherein a first strand of the unaltered nucleic acid comprises, from 5′ to 3′, the first priming site, a first homology arm substantially identical to the first donor homology arm, a cleavage site, a second homology arm substantially identical to the second donor homology arm, and the second priming site.


SEQUENCE LISTING (118 sequences)

SEQ ID NO: 1 - DNA, 2000 bp, synthetic construct
  misc_feature 1..2000: note = Description of Artificial Sequence: Synthetic polynucleotide
  source 1..2000: mol_type = other DNA
tcaatagcccagtcggttttgttagatacattttatcgaatctgtaaagatattttataataagataatatcagcgcctagctgcggaattccactcagagaatacctctcctgaatatcagccttagtggcgttatacgatatttcacactctcaaaatcccgagtcagactatacccgcgcatgtttagtaaaggttgattctgagatctcgagtccaaaaaagatacccactactttaaagatttgcattcagttgttccatcggcctgggtagtaaagggggtatgctcgctccgagtcgatggaactgtaaatgttagccctgatacgcggaacatatcagtaacaatctttacctaatatggagtgggattaagcttcatagaggatatgaaacgctcgtagtatggcttcctacataagtagaattattagcaactaagatattaccactgcccaataaaagagattccacttagattcataggtagtcccaacaatcatgtctgaatactaaattgatcaattggactatgtcaaaattattttgaagaagtaatcatcaacttaggcgctttttagtgttaagagcgcgttattgccaaccgggctaaacctgtgtaactcttcaatattgtatataattataggcagaataagctatgagtgcattatgagataaacatagatttttgtccactcgaaatatttgaatttcttgatcctgggctagttcagccataagttttcactaatagttaggactaccaattacactacattcagttgctgaaattcacatcactgccgcaatatttatgaagctattattgcattaagacttaggagataaatacgaagttgatatatttttcagaatcagcgaaaagaccccctattgacattacgaattcgagtttaacgagcacataaatcaaacactacgaggttaccaagattgtatcttacattaatgctatccagccagccgtcatgtttaactggatagtcataattaatatccaatgatcgtttcacgtagctgcatatcgaggaagttgtataattgaaaacccacacattagaatgcatggtgcatcgctagggtttatcttatcttgctcgtgccaagagtgtagaaagccacatattgatacggaagctgcctaggaggttggtatatgttgattgtgctcaccatctcccttcctaatctcctagtgttaagtccaatcagtgggctggctctggttaaaagtaatatacacgctagatctctctactataatacaggctaagcctacgcgctttcaatgcactgattaccaacttagctacggccagccccatttaatgaattatctcagatgaattcagacattattctctacaaggacactttagagtgtcctgcggaggcataattattatctaagatggggtaagtccgatggaagacacagatacatcggactattcctattagccgagagtcaaccgttagaactcggaaaaagacatcgaagccggtaacctacgcactataaatttccgcagagacatatgtaaagttttattagaactggtatcttgattacgattcttaactctcatacgccggtccggaatttgtgactcgagaaaatgtaatgacatgctccaattgatttcaaaattagatttaaggtcagcgaactatgtttattcaaccgtttacaacgctattatgcgcgatggatggggccttgtatctagaaaccgaataataacatacctgttaaatggcaaacttagattattgcgattaattctcacttcagagggttatcgtgccgaattcctgactttggaataataaagttgatattgaggtgcaatatcaactacactggtttaacctttaaacacatggagtcaagttttcgctatgccagccggttatgcagctaggattaatattagagctcttttctaattcgtcctaataatctcttcac

SEQ ID NO: 2 - DNA, 2000 bp, synthetic construct
  misc_feature 1..2000: note = Description of Artificial Sequence: Synthetic polynucleotide
  source 1..2000: mol_type = other DNA
aaaacgtactacgtccactaatatagtgctcagggcctttaaagttatgaacaggaatacggcgatgacgatagagatgtacaactcagtgcgaaccccagtgtatgtacaaaaagttactaattcactttactgttttgaggatgtacctgccaaaaagattcagattatcaaagtcagatctttatatgacggaacgcgcaaaggatcctattaggatgcgcctcaaaaagccatctaaaaagttcatgtattgagcttattagtaaaggtatcaacaaaaatgattccaccttatataaataagcttgatcccattaattgaataataaagaccgagtaatcacttttatgcatgtaacaaaaatcccgtttgcggctatgctacaacggtcatcccatagaatattatcatcgtacaagcccaagacccgatgctcaacattagagccaaataacgtgcacactcctaatatgagatgactgccgcttttaacaccagatctgttagttaggccacgcacttccaagtttatctagagtgcatgtctttatatatgttggtcccctgtaatgacttataatatttccttcgactgtgttgaacatctgtaacaataaagactaaagctctgggtatataaggttgcagtggtaccttattaggtccattatcgcagaatactgcggatggacaatcttgccaatttaattgactatctattagtttgcacaatataacgattcgtcttggacaaatttggcgagtgagccccttactcgctcaaaatgttacaattgccgagctcggagttgaatgattagttacatattatagaacacaatgcagatgtagttagacaagatgtgttgatgaatgtcaagtctgactggagtaaaggaacaagagcacccacctacgtatattgcgcattttaaatgtagcctcgactctaacacgtgcgacgtgagtcataattgtgcatgttattagatctatggaatgttgtttttttaattatcaaacgtacgtcaaaccgccaaactccgtgtgccatagagtatactcctgaagttcgaaattaggccataaagtctttcttgctggttgtgaaatgaaggggtgtttcataatttaactttgactgcttctgttgggacgacgtacccgttcgtttgtttgtcctactatttagtatcttaaaacagtccatttaccgttaatgttcttaacccttaaagatacaaacttagctctgtaatcaacttcaagacgtctttgacagaacgtctaagacccagatctgtgttagccaactcgtattcaatttcgtaccggtggacttcggcccctcacactgccattagttgatgctgaactttgtatttgctgggtaggatatataacgattttgcagatgtgtgtgctaagtatattgtcttagtgacggtccagcatataaaacacctacacaagaaggttattcttaatggttgattgaatattattaaattgttgcttttactttttcctcctacaaattgtcatgagctcaaatttgttgacctaaggtattaatattgtatcctacacggattgtgaacggtagggtcgtaacaatcgtactttacggcttaaaaattgtaagcaccttgccaggtagatgaaaacttaaaggatagaagtatagtaactcacatgcttgcggcagcatcgtagggcagaggtgtgatcttggtgattgaaattaaggggtaggatgatcggccgcatatatcggctactaggattagatagatgcaacgctttactttaatcaagtgacgtccgtataagtaagacatctaatggctgtatttttgtatacaagtataaggaaccggggagtctttatagcgacgcgtaattatatattccaaatcagttaagtggcgtcggttacgaaactaaagagagtgttcaagacgcaatgaagaatcgtgagcgtaattgttcgcgc

SEQ ID NO: 3 - DNA, 2000 bp, synthetic construct
  misc_feature 1..2000: note = Description of Artificial Sequence: Synthetic polynucleotide
  source 1..2000: mol_type = other DNA
aaccctcgtgtccggtaaaacacgcttcgaatacaaaagattatataggtacggaaggctgggaatctttcttcgatggaactgagattatattccactgtaaccttattatgactatagatttccaacatacggatagattaataccgactgtagattccatacttgaactatgaagccgtacgagtacccatactataactaagactatgacacgtgtgaattcgtgtttatcatagtgcaaactcttgctattccacatgggagtttagaactcagctgttcctatacaattagcactacaaacccactaatatggatagcatgataccatctgaggaggatttggtgttaccatgttgtaatctaagaagtttcacaaaatcaacgttagataaacggcaatatacgcgcactaataatgaaccccaagatatcagttgaaaaattttcgatctcctctttaaattaacaaatattgcagagtaagtaccgaaattgtgacacaagtgccgtttgcccgtctttttcacagcctataaagttcagatctatatgggctcccacttaaccttcagatagataacaagttactggaagtgattctatcataatacaatcaactataacacatccaatgatatatctcgagaaagtcgtagtctagagctccttctattatccggtcttacctaaatagttatatttagttgcccatttaaaattggataggaggaggggtgctcatgatttaaaaaccaactgtgcatgcggttctttgatgtggatccaccttgcaaagcgctaaagataaaagtagtcactacaggaattcaacttccgtcgttgtcagctggcgcgggaacccatcttgtgtaaaaaactgtataaccagacacgtggactcgaccgagaaacagtcagaacctgtcacaagaaataatcttgattaaaggctttcacggcaaacggacctcttccctgctgaagtgtacgattgaatatccacatcgaaggtcaattaccctcatcttttacatggtcataagacaataatctcctatttggattaaaatccgcgcacgaaagataagagtggaatcgattgcattatcgagtttttaagccccatacccgacagatgtgtaaaaagtgtagtggtaatggcgtcaccaagacctatgcttctcataataataggacgtatgccctagctactgctaacggtcgctcttacaatactagctaaaagaaacaaatttgaaaagttatgtaggaagtcattggcggtgaaaaagtgagaaaaaaggtccccggagactgtgctttcatgttatcaaagtacatgccgagtgaagagtttgttttgatcaacttttattatctggagtcattatacgatattgccatggttccttggctgtccaaccaggggtcttttacaccagataatcttctactacactacacctcaggtacgattctttcgttatcaatcgactacaagattatagtgtctctaaggcgtgatgtaggttttccctcaatgacaaagactttacagcaatccggttcaatacgagaattaagtgtgcgagtaacagcaaagtaaaatctaacagaaaggagactcagaaaacaacctattgaggactgtaatatcaactcagcattattgtttactttaaaatctaataatcgtttcgaggatatgagcacggtatcctaacatcaagacaaataccacatcatctaaatacaactggttgcaatgagtcgaatcgcgaacaaataaagcaactataagcacgataaaccactgttatgggaatgataaacagtcttatgacgtggtctatctgtcgtaggtggtaaagccttctgaagatcactatccagttctggcctcaagaaccatttagacagccttttctaaacatgatcgttgctataaggaccggggacacctagacaaactcacggaagggataacttacatc

SEQ ID NO: 4 - DNA, 2000 bp, synthetic construct
  misc_feature 1..2000: note = Description of Artificial Sequence: Synthetic polynucleotide
  source 1..2000: mol_type = other DNA
actgcttatataggaggtacaaacagatacaatccttagttaactagagagaatgctttttttcgaccgacacgcttataacttcactgggcatggtcaccatatttaggtaaaacaaactgctgcgctatatgtcgtacacatcctgagtgtaccaatatgtaggtggaaggcaagttcaatgagacgtcagttaccaagcaaatttacattctagcagttataaatgtattatgacgcagttcttgtggtgagcgatcatttacattaaaactttattcaagagcgtatattagcatatattttccggagagtgcactacgggccgaaatttaggctggaactccgcaaattggttacgaccctgtatacatagttcttattattaagtaaaatgtgtgaataaaacctacacgacgcgtcatatacgtaaaagtttatctcttgtagtaatcaactaaattaacttactactatctggtcgtccgtatgaccctgtgagcagattattttcgactcgacatctatgaattctacggcacgaaaagttggtaacttgtactgggttaaacaatgtgtattcgggagtctgcggaagaacgtttttaatgtaacttcctttgcaaaccaaaatttggtctattcaaactgacactagcgtaatctataccgcatgagatcctgacatgatcctatatctatgcgcataggtactcgcaccaataagtgggtcgtagaatttcacgtaactcaatgttgtctcctttcattttttgttaattcgagaaaactacaaaaatagttagtaaaatgctcaaggagtcaggtgctacctgtggaatacatctatgtccaatggaacttgctccctcggatgtgcgatttcgttgttcagttgggcctttaaggaatacagcaactccaactctttgattttaggtaagtatttgattcgcggaaagtacagtgtataatctgttatttgccaagacgtcatcgaaatcgagtgtatcgagatcagaccatcgcgctatcgcaagatatgaagagcatagacagatcacgatgccaatcagtgtcgatggtgcgaagacgcagcccctgtgatcaaatcgtccgtttctcgatttactagcggaaaacaaaaacgaagcggtgaataccctgcgagctaatgtctttacccggttatacgagctgataactcggaaaatgctaatatcgaggctgcgcacttaaaaaaatactttaataatattaataagcatagctgtatcataacttaaaattctactgtatgatttagaatctaacagtgttaacgatctacagaccgcactaagatgaagacggactaatctcctccctaattttccttgttgattagcaaagggagatccttttgttatttgaggtttacgagaaagatgtaagagtcgaaataattacgtaaacctcatagtcgtcacctagagcaactataacatgaaccactcgccttggttaaatataaaataacttcttctctgtaacattgttgcacacaagcgagcgacaaaatttcacaacatttgttgcgtagataatattactgcatcatttttgcgtcagagtgaatgtcacttatataactaggaaaaattagtaggatagctcttgcggttgagagtaatgtcgactgaatcgaccgccatagatggtagagggagtgattcaaatagattaatgtatgcgctccatctataaggacggacaaggatcaatgttcccttatacttagctaacaggaccctctccgaaggtctgataatgcactcatataagcatcgatgcgtcctgagtagaaaaatctttacaaacttttaatagataagttatcttggaggtgctatctattcaaatctctgaacagatctgcggcatgataatgtctttgtaccggtgtgaataatgtgagtcagacgtctgtgcgaagtgggaaccgaaatcttttaatcatt

SEQ ID NO: 5 - DNA, 2000 bp, synthetic construct
  misc_feature 1..2000: note = Description of Artificial Sequence: Synthetic polynucleotide
  source 1..2000: mol_type = other DNA
gattcggtcgcgttccataatcgaacccttaagcccatcttccagctgttaacgttatgtaccatcttacctcaatgtcagcgatctatgaggttcatgtttttggtggattaaaaaacttctttatagtggtttagacagaacgtttagcgctgcgctcgaagtgtcttatctaacggaggactaaaattacctggtcactccttagacttttcgtagtacttaattgccggacatccgttgggctacaccagcaagaacacaaagtggtatgtgtgaagctagactgacctcatgattcgtactacattataagaatcaagcttcccggatttgtgttctgagatattaccacgtacatttttaagggggttcttgacatcgtaacgctaaggctgattaaagaggagggtgctatgcagagtttattggtgtttcatcaatgtatcacacaaaattagctactataggaagtagctttggtgcgagcagggggcggtatggttaagaaagctatggtaagaaaggcccaggtgatactacgtgtaaggttgtgaagagccacaagagccaagttttgatattcgacttcctccgaatctacagcttatcgagggttaaacgttacgcatattacgagattacatgatagcttctcagttctagcacatttatgagaccctttgaatggtgtcaataaataggaggtccccatatgacaagtagaatactaactataagagatttgtaacgctggataccatttgcagaggattggcccaaagaatgattgcccaacgcttatattgtcagaccttgcattagaagaataacgcagaatacgactgcagtttgatataattttggctctgggttgccttagtatcattactaatagacttgtggtctatatccatttgtttaatggaatagactgggtaaaacacacctcttccaggctgtagttcttcatgttgtaaggatccgtcatggcgtgcaaactaggggaggtattttttgctaattgcggtaacggctccagttgggatatcgtcaatatgtgccactcggccctttctctgagacgctaagatttccgtaaggtatagcgataagagtctctaatgccagaggaattgttaccgcgagcaagattcatgtctatatataaaatatcatccactttgaattactggttggaatcatcgttcgcgttataacaaaaaaccttttaattatgttaccacagatctcgaagtcccttttgaggcagaagtttaaatataagctctaattgtcgcatctaacgggtatatcgtctcaacggtaggtcaaaaacatttgttaacttcagactgtacattcgcatttaactcgccatgtaaaccgcaatacatctcgtgcctatctctcctagtaacgtattatcgctgggtgaaagcgcaactaagtaataagtgaatgtcattcacaatacctaactctatccgacgcgtaagagcgacccagcagtttaatgacatgataaatcaaattctatgcaaggcagtacttgctttgtggacgatagcgattttccaccgtattgcgaagtcagttatgctgaaattttattccattcgcataacaccaaggcttactcttaggaaaaaatgtaataccgattttggtatgaagtatgttacagtacagaatgaaatgcccggcggcgtggtcaaactgtttcctgaggttcatatagggaaaggtcatccctcagaattggccccgtaatcgcaaagcctacgggagctttcttaagtccaaccggtaaagccaaatctcaattcatatgaggaaatgtttgaccgataaagaatagattgtcgaactaacagtcacagagaaaatacgagtagcatcacctaaacaaagcaggtaataaaatagactaatggagatcatcgtatcggcttatgacctgcgtccatttaaaggcaatgaatacattaccgactaga

SEQ ID NO: 6 - DNA, 2000 bp, synthetic construct
  misc_feature 1..2000: note = Description of Artificial Sequence: Synthetic polynucleotide
  source 1..2000: mol_type = other DNA
agttatgaggttcacttctcatataacactatcaacaatgatcatctcttgcgaaacaagcgccctacacagcttcaatggaaccaagagccataatgaggtaagggacggctagttactaataaaggaatcgattttacaaacactaaatgaaaaacttgcgctggttgcaatgctataaaaaaatgaaatgcaaaccagtgaagatcccgatcaaccgttcgctgatttttattgatgctgtacgttgtgttagtttaatgatatataggccatctccaggttacttaggacgccaaaattactattttgaagctcaaccgtggtataatagctacaataattaattgatgcctgcaggtcgtatctcgaacgattgtacgcattacctatgatatgaacagaatctgtatcccatacttaaaatcttgaccttgtaaagatttcgcatacgcattaagaaatttcgttctacccgcacggattgtccaagtatatctggccattcacagaagttactaatcttcatctctaagtttaaggccgacaaagggtccaaaacctgcgtaggttacaacgcagcttacactcagtgactaaccaacgctcagtagggtaactggacttgttctcgctattcagctggtactgtaatgatcaacttagaacggccctatggctaagcaaggagtacgcaatgttttagaatacgtgtttgctcacacaggtagtagtttaatataccccctgacaagatatgttaacatagatgaagtttggtattacttatagccagactattcttcaacatatacactgggttttaggagtgtgcaatttataaggacagttatattcctacaatcgttgtatgatccttttgggtttggtagaactacgtttgggccgcgcctttggtcaaccacggactttctgtctagatgccaattcctacaagcttagtcctatcaatttagtagagaacaaattttgtcatcactgaattgtcgtcttactatcggatcattctccgctaattataggattattagtaacgcgtatataggagcgattaatgactcatcaatgaatagcatcactaggtgtattatatgaacctctctctattctattaactgcccactgtgggtaatttgagttatacctgaccggtccctcggatccttaatcctttgatgtcgataggtaactgaagtgtaagatcctgatatatgaagccggtaaggagacggagattttatattagtgttcttggatactgtgctagaaggttctactctaactcaaacaggttataaagtaggaaggaaaaagttgatagtggtaaactaattatgagttggcttgcttattccaagttagcgaggttttcatgacgtaagtctgataaggtttgctggaagctgaaaagttttacaaaaacgttgttttagaatggtttgtccccgaaaatcgaacctggcatagccctcaggagacgaacaagcccaggcaaaccgggggtttctcgcttattgctataatcacctctagtgttgtagaagcaattacggtggggaggcgtcaatgtggcctgagttccgttgaggacttttcacgtgtaggacccattaatagaggagatatatgtctttcagctgcggaattcataatagtggaaagaagaaaagggattactagattaatattactcatcccagacttaagttgaaagctacatcttcacacccaggaaaccggaccgcctttgttcaggtctaagtagtctggaacagaaccgtatcaactgccccaattcataggtgttagcgtgacagcgatcgcggatttttagtccagactggctgggccatccgcttcaataagttagaggactacatacaacgatggacccaattggcaatagtcgtggtaaacttcgaaggggcggtgtaagattcaagctgtagtcgtgatgaaggagatcatcgtataaacag

SEQ ID NO: 7 - DNA, 2000 bp, synthetic construct
  misc_feature 1..2000: note = Description of Artificial Sequence: Synthetic polynucleotide
  source 1..2000: mol_type = other DNA
atacatctagactactaagagggattatcccagcgcagtcccacccaaacatcaatctgtccctttgttctaatatatctctggtcgcgaatgagtaaacggggctaaaggtccattatttttatgtaggagcatgttgcttattatggcatagcagtcgccatccccctgtcactcgatctagatacatctcacattgattggaaacttctacaaaacgttagtacttaagatgagtgatttagtgcatttctcgttttcacaaactttgctaaacaaacgtattgagtggcgcgttttttgatttgtcgcataaccgtttactccctgttcgaaggaaatcgatctccttataaataatgagtacattatacagctagcataatctgcgtgtggcaaaagtgaacgtttaatctacaattgatggaaaaatagcccgttagtccttttaaagacgtcttggaaaaatattgagacaaccttcgtccaaaatatgtcaaagcttcgtcacatcttttcacctattactaactccgtagttcaactgactttagagggcaagttttgagacaatatcttagggctgactaataagacggttatatttcaagaaggaaagatcttaagagtcaaaaaaacgtcagggctatcgttacgatattggtatgaacagtaatgatatattttgcagatcttaatataacgacattcgaacacaatagcgtcagacaaaggttaccactcctctataattactgcagcttcaattgatgagcgtcatttaattttggccggacatttacatcgtgagctggcagcacgctcagctttattgttcttgccagaacattacgaatagccgttcaatgccaattagtatgataaaagtagtgagtgtaaaacatggcctgggtttaaagaatgagtaactattattttgtaggaataactgattcccttgagttctatcttaagttgtacagaatcacactcctacagcgaataagcaacgacatagaatccgttatttcgtatgtctcggcgggacatgtataagtagcatacgttatatcggttgtcgcacgaaccgccttcattccaaaggcgcttacaaatctgcagtaaaaagcttagcatttactatagagtatcggcgttgaccgttaagcccgtcccgtccattcaatcactcaattgatcatcttttggcaatagtcgtcatatgagaaaatagctctgtcgttgttattattggctagagtataagctgttaaactacagaatgacgttttgtggaaagtggacgtaagatccttgttcgcgaagactcgcacggtggggaacaattcctgggaatatttgatctacgtacggttattctgcatgtgattacaatatttccaacgcagtccttttgacattatatgaaaccagacccgatgcatatgttttctgactggtggtttgagtcagagtcaacaaaagtatcagtctttcgttactaaatcttcctaagtaaatggtgggcgaccattccttgtaacctgttctgttataggtactattccagcctggaaatcgtggaacacatcgatctagttgtctatctataagagaacactcggttccaaatatgtaatccgcacgtaagagaggagtctcgtacatgatatataacgttgggtacatttcttagacattccggtgatacataatgtacaagtcacatgattacaccagctggtagatagaatacctgagactgggtcctagatgattataacaagtgttacatggacgctctcgttttgttgttggcttaacaccagggcttgctccatgttctcatgtcgttattactgaattatcttccattatgatcctggacggatgaacgaagcagaagataacaaagatgactgaatgccggaaaaggaattaggccctgatatatcgcgcttctttatgcatgtttacgctgtaccaataaacgcaagagg

SEQ ID NO: 8 - DNA, 2000 bp, synthetic construct
  misc_feature 1..2000: note = Description of Artificial Sequence: Synthetic polynucleotide
  source 1..2000: mol_type = other DNA
gtacccgtatatcgtcacttcatttgaagctattattaatgtaaaatccttccgtcacacactcttttcaaaaagggaagtctaaattaacattcagatgaaaagcgctgacccacatgggaatatcctttctacgctatcagccgaaaagctccagcgattagctaaatatctaagcctccagaacagagttattatatattggttcgaatatgctaatattacagtagaaagtaaggtaccggcacttttaacgccgaagtcgaccggtgtagctgtgaaaatatatttagtacacgtaatattaattggaaattgatgagatcgaatcttcaggagaatctgacgagcattactaatcgcgcgtgacgggaacgttaatatacaagcgtctattctaggttataataaactcctatctggcaagttgaatggttttttcaaaactttaacgttctggctatacaaagctagttgctttaacttatcgcatactatgatccttcccatcaatcaatctcagtgactataaacgcaagtgacacaattgtctgcgttccacatttctaaatctcttatcgctcattccctctacacaaagttcgattaccaaacgcgggtctacacacaagcttacaaggattacaatatccaattttttgttatcaaaggcgaactcaacgaatttaatcgttggtcattggtatggaatggcgattataagaaaactcttttagtcatagtagctcgagatgaagtgaaccgggccagtcggtagtttcactatcgcgcagtagtcacgatcagttcttagaatctatctcctaatcaagtccaacaagcaatccgaaatgttgctttctataaagggtatgtgtacctgccaatattaaacttgattcactcaatagtgattttaaatatgtccatatttatgcaagaatcattgacattagtaaattcagccgtgcatttgacacaataaaggtagatttagactgcatatttcccgcatatttattattgtcaacgcacaaagttgatggaccgaccacgatcgcatcgaagaccgtctaaacgacgatattcttcggagatccatatttgttttcaattaccgaccattgttcatcaagtgtagttcagtcggaaatttttcgtgtgctttttaaaataccaaatctgaggaaaaagctcgctagatgttgagtcaatccgtaagaatatgccccaggagacatatgtaagtcacagccgtagactctcggttaccccacgatatgttccatatgcaacgtttgttgagtaatatgcagttcagtcgggcgtattatcaacagacagactggcacagtaaattttatcatcgggtttaaaatatctagatacctcagtttcaagggggagttgaactttaacacgagatcaaactacatacacaagattatcagtgggtacgctgagacttatccttagcctggagagagtccagctacaggaactgctagtacttagcgtgcgacctcaaatcgagagaactaattaccctgatcgacagatcgggcaagttaagcaaacgcggctcgcgtgtagaaccataacaattggagatgctcctgcttaagagattatagaaccgcaacccatcaatcgtcagttacccgagggctcacgcacgcggtgatggaagttagttcctttgtacgcacgagctgcaatacgtggtgattataatcggcgcacactaaaggggtggatacaatagtagaagcatatacgtcgcataggcgtacgcgggcgaaaattttaatcgttaacgtggcactaacagcgttttgtctccccactcgtgggttgcggtgcatcgcacatattcccacaacacctcttaatgctttattatttgtattaatggcgcgaatctgcctgatattagtattcgcactagtgggtaacgaaatcttagtcgctggctactgcagaactaattgcgttgcgat

SEQ ID NO: 9 - DNA, 2000 bp, synthetic construct
  misc_feature 1..2000: note = Description of Artificial Sequence: Synthetic polynucleotide
  source 1..2000: mol_type = other DNA
actagctacagatctgtaatagaaaaatgcagatgcttgttctgcgtcgactcgctcatcaacatcctgtctcacaagttatgcatcctgtgcattttattgaagctttgatggggattagatcgtgtatggaaatgtttattcgcctggataagatctgtcggcttattcgtggccaataataggtcaatttgcggaaacataaagactcgcataccaatactcgcttatcctgaggttaaatttagtgtatgtagacgaacaacagtatttagtagtatgacgttcccccgtattgccagaactcctgaatatttggatatgaggtatgactacgaaaaaaatactacgttgctcataaccattggtgcagggataccgaactcattgttaagggacgccacagtccagtctcttttcgttcagagcgtgtttttcaaagtgcttgtattagtgtggacagagtttactgatctctccgcacttggactgattgtgatcccgatcatctcttttcataattgtaacacgctttcatagtacacttctgtacattgaagagtgcttgcagccggacagtcctatagaatttggcgtttgttcggccaatgtgtgcattttaactttaggcgccatctcttgagattactcctttgaaaaattttggcggaggttaactctggtctttaacataggcgtgcttaacacgagctttacggtcaggtacaggtaacaaaacaggtctaaatttatttaagcagcttctgatactttccaagggtcacagttggggagccttccgaggtatgacaatcagttttcaaaaggtgtagaatatcatatattctatctaggccagagcattctaagctgttaaaagagtgctatgctcagaagttgactgttctaatcgaaaatcggacatagataacccgcataccacaagtcccgttgtaacgtacccatcgtttttgattctatgtctttgctaatgattggcgattgagacatcctacttctgtagcttggctgttatgcgatccaaaatggtatccagtggtggatgtccgccgcaaactgaaactccctatcagttctttgaaattaatttgcgggctatccgactcattctttaggaattaacagaagaacacgcgtctgtaccaaggttcttctttgttatatcacataacaatgaatcacgttctatgatgaatccaggtatagaagttgtaggtaagcacttgtataagggggcgctcctctcagattgattcattatttactaaaaaaggagcgtgttattacttctaacaactcctcgccattatatattatttaactaccattcccactagaaatggatatcgtgttctaagaccctaattgtgctcattaaactaactaccgcaccaaccgccttgaatcaccggaccacactagttaagctgccgatacccaatatggtattttagtgtataccggatatgaccttatttacgaatggattgagctcaccccatagatcagtaccagcgttattatgaaaatcttgttattttaacagagagacatgcttggtcattactacgaatttgagtttacgttatacaaggcgatccaaacggacaatagcgcgatacgagattatagtaccaatagcacgaatcagttttagcgatctcgtccgatctgtcaagccgaatgactctgaaacgttagtatctgaaacgtttcattcagcctaagatatgtatagtatcattataccgtgtgggtagaacaatcaaatgcagataaagctatttaatgcacttcacataacctctccgttggaaatccatgtattctctaatcaattgaattgtaccttagaaagcacagggggacacctgaagacctcccatctcttaaggttaccggcacgtgaaacttcaaaagtcagacaatcaaacggcaacgtgaatgtcttcggaagtggtggtatgcacatcgcgtca

SEQ ID NO: 10 - DNA, 2000 bp, synthetic construct
  misc_feature 1..2000: note = Description of Artificial Sequence: Synthetic polynucleotide
  source 1..2000: mol_type = other DNA
ttaatagaagtaataagtgctattggactaaaatcgcgtcaattagctatagaacagctctgtgacgaactatcaatggggcattcgttcactagtggataccgtacaagctcgccgtgatcgtgcgtcaaggatagtgccagagcgccgcgctatatgtgtaacgacgcataagtagatgtttatgttattgggcaaagtcattcttatccataataagcgctgccgataaagattcatcagagatattgagattctccatacttgactaatctctgagtaattaaaatatatttctaatcggataagttagggatcaccgaacccaatgaacttagtttaatgtgttctcgcgaatatccccatgatataaagatccgaatacctcagctccgtgcgtgctcgtgcagtcgtgcgttttctatgaatcaaccatcagtaacgagtagcggtaactacttctcgagtttaaccaaagcctatgtatactagcgtgcaatcacgtgcggaaggtccgacctacagcagcattttcgttcgaaaaacgaaaactaatgtgcactatgttgaatgggcattcaggccttaacttctaacgttaaactagatttgcgattattaggtatgagatcgaccaggtcgccacagataattaaagatagccctagcaaagtgataaggtccggatgttagaacttgcaagagtgtgtaagattatttactctcggtgcgtcgacaggcgaaacccataacttttatcggtcaagattacgaccttcagctagtatcttgagatttgaaagggcctaaaagcaatttagtgtacttgtgtaacataaccttaattattgatggttctatcgactcccagcggtaataatcttgtaatattgtcggatttagttgaagggcaggttgacataccgaacaatagctagtatcaatgtataactagcaggcatctaatttcgtaaacactcctgacacttgtcgtgtctaagcatgttaggacaaaagaccagtttttttaaacctgactgtaccggcaacgccacagattttatgtctcgcatacgtacgaactgaatttgagggggctcaggtttggacttacaccgcacgtgactatactgagatcgaggctccattaacggcaacataagactagcactgtatgatctgaagccaggctctggtgaaattgcgggtagttaacgacatttatcgacgaacccttgataaaaagtgattatgttgtatctgcgtgatatattcttttcgtgttcagtctctagaacttcgtgcgtaataaagattatagaggaacggttaacctcattacaagacggagaccgttcatagacgccgatggattacagggtctactatagctacctagaacactggtgaacatagggataacatacaattaacaatattccgagccaaattatgtcttgagtcttggttgttatctatatcgttattatgttagaaactaataaatgcgataagaactagattttacagtagatccaaataccggaatctatcgggacgattgattaagacttactcaaacctaactttagcccgattttgcaattagagatacgtcgatttcgagacaagagtagcgtccccatggcaaatatccacggacagataatgacacgtgagggatggcaagagtagttgctcaggatgtaggcgttgatggtctggcgctaatgtcgtggctacctgttgagtctcgcgtaatgactagtagtgttcgaacgtatgaccaagttccttcctagtgttaccactttgacacatacccaggggtttgccgcatgtcgctactatagtataggtgctgctatgaagcttctgaatcagcggctaacaagtacctaagaaaattggacatcttttggatgacagtgcacaggagcctatactgaattatcggtgatcgatgcttcatgtaatcaaaaccagcgcgtacacacttt

SEQ ID NO: 11 - DNA, 2000 bp, synthetic construct
  misc_feature 1..2000: note = Description of Artificial Sequence: Synthetic polynucleotide
  source 1..2000: mol_type = other DNA
tactcttaattcattacatattgtgcggtcgaattcagggagccgataatgcggttacaataattcctatacttaaatatacaaagatttaaaatttcaaaaaatggttaccagcatcgttagtgcgtatacatcaagaggcacgtgccccggagacagcaagtaagctctttaaacatgctttgacatacgatttttaataaaacatgagcatttgaataaaaacgacttcctcatactgtaaacatcacgcatgcacattagacaataatccagtaacgaaacggcttcagtcgtaatcgcccatatagttggctacagaatgttggatagagaacttaagtacgctaaggcggcgtattttcttaatatttaggggtattgccgcagtcattacagataaccgcctatgcggccatgccaggattatagataactttttaacattagccgcagaggtgggactagcacgtaatatcagcacataacgtgtcagtcagcatattacggaataatcctatcgttatcagatctcccctgtcatatcacaacatgtttcgatgttccaaaaccgggaacattttggatcggttaaatgattgtacatcatttgttgcagaccttaggaacatccatcatccgccgcccttcatctctcaaagttatcgcttgtaaatgtatcacaactagtatggtgtaaaatatagtacccgatagactcgatttaggctgtgaggttagtaactctaacttgtgctttcgacacagatcctcgtttcatgcaaatttaattttgctggctagatatatcaatcgttcgattattcagagttttggtgaggagccccctcagatgggagcattttcactactttaaagaataacgtatttttcgccctgtcccttagtgacttaaaaagaatgggggctagtgcttagagctggtagggctttttggttctatctgttaagcgaataagctgtcacctaagcaaattaatgctttcattgtaccccggaactttaaatctatgaacaatcgcaacaaattgtccaaaggcaacaatacgacacagttagaggccatcggcgcaggtacactctatccacgcctatcagaatgtcacctggttaatggtcaatttaggtggctggaggcacatgtgaagcaatatggtctagggaaagatatcggtttacttagattttatagttccggatccaacttaaataatataggtattaaagagcagtatcaagagggtttcttcccaaggaatcttgcgattttcatacacagctttaacaaatttcactagacgcaccttcattttgtcgtctcgttgtatatgagtccggggtaagaattttttaccgtatttaacatgatcaacgggtactaaagcaatgtcatttctaaacacagtaggtaaaggacacgtcatcttattttaaagaatgtcagaaatcagggagactagatcgatattacgtgttttttgagtcaaagacggccgtaaaataatcaagcagtctttctacctgtacttgtcgctacctagaatctttaatttatccatgtcaaggaggatgcccatctgaaacaatacctgttgctagatcgtctaacaacggcatcttgtcgtccatgcggggttgttcttgtacgtatcagcgtcggttatatgtaaaaataatgttttactactatgccatctgtcccgtattcttaagcatgactaatattaaaagccgcctatatatcgagaacgactaccattggaatttaaaattgcttccaagctatgatgatgtgacctctcacattgtggtagtataaactatggttagccacgactcgttcggacaagtagtaatatctgttggtaatagtcgggttaccgcgaaatatttgaaattgatattaagaagcaatgatttgtacataagtatacctgtaatgaattcctgcgttagcagcttagtatccattattagag

SEQ ID NO: 12 - DNA, 2000 bp, synthetic construct
  misc_feature 1..2000: note = Description of Artificial Sequence: Synthetic polynucleotide
  source 1..2000: mol_type = other DNA
ggccctatagattttaacctaagctctagcttgtgtgtgctcagagtactgctcataaatatgctcgataaaggaggtaaggcatatcgtaatttggaagataataccacacttattggtaacacgttggaatcacatattaattatgagccagccttggcattcgagcagggatatgtgggagtatcagttgagtttggctccttgctactgccctctgatgctctgcttgctctagcttaggtcattaatgataaaaaagagccagagtgtgggctaaacaggcaacggtaccgttgtagagcgaggtattgctatcgggagacgtcgggtcaaagtgggattcatgcagtaagtttgccaaagggtctgcttaaagagaccgattccggaaggctatatgccatagcaaggtatgcactgcattgagctgaaaactcttgagcatagtatttactaaataaagaatctgatatcttctagcgtgttcactggactattatttagatggtcgccaacaacaagcgtgcgaatcatatagacccaacccagggtggtattgaattctatattaaaatgtctcgcccttataactctctaggtttccatagtacaaacctaggtgtcgtcaactgcatgcactgctttttgtatcggtaatgttgatcgacccgatgggctttttttaataaaggtcttgtttagttgatcatactaccaattttggtggtcgatggctcaatgaccaatggaatctttatagtaaaagagcccttggcaccaacgaatcatggaatttaggacgatgtctcatttaccatattttgcattcagactatgactttcaataatagaatatcatcgtcaaacaccgtggatatggcatcgacaagtgttgggatgcccactgaataacgtctcttcgtcatctttagggcggctatccattaaggaggattttatttttatagcagtcttagtccgaggcattggcgccaaacatcggctcaacactagacacgtctttaatggaaagtatctagtgttactgcggtacggaaagcaagttcagtacttttatccaatctaagtatcacccagcttatatttaaaagctaggtaatagggaagttactaataactcatgcgcgtgtagtgtagtcttgctgtcgcttaaagcaactgaatgaatgtacggctgacaaaggcttacccaagaaaactctcttgtacgctacaagaaacctgtaacaagagaaaaatattttagcccacgtatagtgaggccaaacttgatgcccgtaaaagcaaacaagtaatattcagcagaatttgcggtcattcaagtgtttaggtacgtaacttttacagaattagctgttgattaggtaatactaaatcaaaatgtcgtaataccgaagcagaagtatatgatctaatttgtcgcctcgcttcatgctacgaatgttacttcgtttattacagctgcaaacttgcagtgacttgcatttgataggattcttcctagggaaccatactgggccgcggacagggagtcaggaactcataacggatgaagatgtaatctctataggggtgaataacaggattgaagatagtaatctaagtactctcatctcgtggacgactttaagcgcactgacagcgactcgcgattcgacgaacacccgtgatcgatttacacgttcattctgaaagatatacaggtaataattctaaaagataattgagtaccaatatataggttttatgatcttaggcgcatgtcactgacgagagaaaagatagtcttgccgcctctaagtgttctatttctggacgtgcctgggcattaagggcgacgttgacttttatacacatttcatgtccactaacaattttatatcacgtagcaggacataaagggaggactctataaaaagtttcgctatatacgtacagtacgttcaaaatctccagaggaaagcttgtaaaaaaag

SEQ ID NO: 13 - DNA, 2000 bp, synthetic construct
  misc_feature 1..2000: note = Description of Artificial Sequence: Synthetic polynucleotide
  source 1..2000: mol_type = other DNA
cgctcgacacgagtataacaaatatcgatagatgctatagtgataaggtataagtaaaatagtactgcgaatacaaatagcttggagaaatacgttcatcctttaacttcaaaaatttttggacctcaggcacgttgtcattattactggcaggtgataccacccaaaaatcgtacccgcaatatatcttcggtaattcttgccaagttgggattttacatacttagtattaatagtgggatcagcttcgatcgaagaccataactcagtatgtgtattcctcatacaagatttctgaaggacgaaggctcatcaatgctgaggtgttatcaggtcaataacaagccgcattaacgccgtaaccctaatgccataattctttgacgaaatgccaaatagtttcatcaggaatcacattatttggataaggaagcacaacaaacgctttaatctatacccctagaattaagaggacagcatgataggctttgcaatgaaccagtctcctaagcgtaccaccactccggagccttatggcgcgccggtattatggcgatgcactgcctgggcgaaactcgagtgaatcatttttcccgatatacacagcagtacgccgacggtctggtaaaaaaaacgttataggctttgaccgcatggtgatcgtggttaagtgcctttacctagagtgctgctagatgtaacacaattgatctgacagtttacgaccttgtaatccaagaaccatatagatgacccgctgagttagtaagataatgcacgctccggggctaaatctagtgcggttcatgaataccgaatcaactacggttattggctgcggtagaatatttagttgtgttaaatatactctaagatgaacatgtatcactataatcactcaccccctctgcgttcataagtaagtggctagtgtgatagtaacttgtatcagcgaccactactatatgtggaagcttttgaatgagaatctccgcacatgatgatgtattgatacaattcttttgttcgaaaaagcttcggtgttttttaggacaggagattaacgctttagagtcatacatatatgtcaagaaaccggggaaaaaatgccagcccagagtgttctaaacgataggttgttcagtttttaataacccgcgacgcgtcaagtaacgtcacgggtcagctacgattaccaatttgctataaactttcccccgacgagccaaatccctcaaagctgccagataaaaggatagcaacctgtactccccgtcaaatctaatgcattcttgttttttaagtctcgtgtaacatgcgttggctaatcttctctaccgggtccagtgccctttcagcttatgcctcacctttgattagtaatggacatcagcttttagtcacatcggagtgccaattataccgttatatctttctctgatgcagaccgacctgtcgtgtaccgattcatcctagggtaactagccgtggcaaaatatctttatcgtgttgtcaggacttggttgttatatactctagcccgtagatttaaaataaattaagtgtagatcgtccaaatatctaaagcaatcgcagtttttatcacatcatgtgttaaaatgcgatcaaaagaaaaatactgttatttcgagagtcaaggctgtgaggaaatatgatgaagactgccatcctggtggactggcggccccaacgttgaagtttctatttgatcggttattaaaggatactcgagaacaacatcgaaggaataaacttttatagaaagtctccgaaatgaataacttaagatataaatttatcgcgcgatagttctggtggatgatagctttattcctcttaatgcagtatagctattgcacctattaatttgtataataacgtatcatgttagacggtcagcatgatattccggatagtggaagcaaattacgacatctaaatatgtcgctagtatttgagtcattatagcttcgaggctt

SEQ ID NO: 14 - DNA, 2000 bp, synthetic construct
  misc_feature 1..2000: note = Description of Artificial Sequence: Synthetic polynucleotide
  source 1..2000: mol_type = other DNA
ctctaacgtgcatttcttcgtcgcctttgtaagaccccacaaaaacatgacgctttagggatatggtccaagactccgaattgaaagtatgctggtatgatatgggacgtttttgaaacccccctctcacgcgggtaattgggtttttagttagtgtatcatagtaggtatatctacgaactacgtctgactgagagagactttgtgcctctcaaccgctatggtgtcagcgactgatattggagttatttacccgtcgttatacgtgggtaatctttactacggttcaaggtaactaatctagtgtaggtagaatgctgaagaattacccgttggacccggtagtccgtccgctccacgcatggaatgcatgagtaacgtctaggtgaatatccggagtgcataactttttggtatctagtccgctactggatgcagaatgacatatttttttcgagtgcttactattactcttctcaaacagaacgatcattatgttgcttaaattcacgctatgttctcgatgtaaaacaattttcgtagagaaagatgcgtaaaacgcagagttagcatataaaaagtacaatcaagcccgaagcactcacaagaaacataggggctaaatgttaccgtccaagtgagtaggatttaatatcaagccgggcttattgggtacagtacgtggacggactacgacgcatgtgtgttatagaatgaagtgcctacaactgaagcacaattactaaaggaatgtacctgggtttacactaagcatcccatcctcttcgcggttcagcctgatgtaaacgtaaatctcgtcttcccattattaagacgcctcgatctacgataggtgatacgtgtacatcggtggaccatgtgttttgatattcaacgatgtaagtatggttccctgcagtgaacccctcttcaagtcgtcgatgtacctgcaagtgtacaatcggaagaccatgggtccatatgtaaaaataagttaggggtcttttggtctgtgttggttataatcgatattgccaaaatattatggacagttagttcgaattttgtgtatggtagccgtcgaaaagggtggacgttaagtatatccatcccagcggctgggagatatgtagaccgacgagtgttaagttattccacttactttaggacgaaatcaatacgattattttacatcggaggacatgacaacaaaaaactactcggtttcgacaggtggaagatgtcgctgcgcaccagtagagcttaggagagcgacggtactcatttgcagcatgggtacgtaatcacgttagtaaataagtaagtatgccttctcttatgtcattttataagctataatggtgttgtgccaacttaaagattgacacatgatatgctaccagataagcctcgagtcgcctatattttgctactaaacctgattaactagagaataggtataatccctggtaaccagtaattttaatactatgttgccacttgatgtagacctggctgtggttactaaggtgctttgaaaccattgaccacccgtttctgctcgggttgtgcatctaacgtaaatattcagagataacgtggctctgctattatttttatattgcctgctgacatatcatcatccttgaatggccagcaacagttcttgatcggcagaggccccatgaactagggtaatatagcagattaactatcggttaactgtattaaacttgtgtaatacttatattgactaattgggattgcctttgtcgttatctcgtttatcttgaaaacggtgatgtttttagaggcgatagtattgaatagctcgaatgatcaccagccatcaagaatgtagctaactccgaaactccttgacgagagctcaagcgaatactaggtcggcgctgctatccgcagagttcagggttctacccggggtataaaatcccattgatcattcagatattatggacttggcgtttatgcgacgagtc

SEQ ID NO: 15 - DNA, 2000 bp, synthetic construct
  misc_feature 1..2000: note = Description of Artificial Sequence: Synthetic polynucleotide
  source 1..2000: mol_type = other DNA
aagaagcagctagtgctacttcggaatagttgtcgtttaagtccgttcaaacatgacgctctagtcattttgaaacctaaaccagtaataatagactgactcagaatgattatactgctatctctagtttaaggagatccagcgaaataacttggtgaactatgccgagatactataaaaagatcaaggacgggtcgctcacggttttggtttattttactacttcttcgtggctgtattagtcgatgcaagttctaataaatagcaaacgttttaagtgggattagtacatattgatggacgtccaccacgtcaaatctcgcagcgtcatagaaggagctataaccattcactgcgactacgacatgtgtttgggtagtgccaactacccgcttccgcgtccctgccgttctgtacacttataaaattgatattttaatcagtggatgtgctgatacggggcactgagatgatgaatagtattaggctgtagtaccttatgtacgcaagaaattttagagtaaagattagtctgtgggtaaggaaaaagctaagttatgattatccatggccatggcatctacaagctgatgaacgtaccaacattatctaatttaagaacttaacttgtcttatcctctcttaaagtcttaatttgcactattaagcttagggaagtcgcaaccaaactcgtgtagtattgagataaattattaaactttcttagtatctactgatatccgtatcaagtatgcttataaattcttgttctgcctgacaggctagtgaatcctgcacccgggacgattgcaggtgtatacaggccctcacgctagcaatcaataccaatacgaaataagggctaacatttttcgtaacagattagaagcagtcccgttcagaacttaccactgcaccaacggaggtactgaattcggactcatagaatcctcgagtagtaagaccgtagaagagacagtgcatattaatgtcatagatcaatttatattttatatggttgcccatttcatgatacccctttaaatttataacttagaaaaggagccgcactaataatgagcggcatgctgtaaaaaagtaggccaaaacgcaagataaggtacctttgttgtccaatcaaattaattgatttattcttcgatcgatcgaccgtcatagttgaagtaactatttagttacggcagatacagcgtatcaattcattcggtgactttgcttagataactgctcgataatccggaattatcatcgttcaaagtccttcccttactaaggctcttggattcagatgatcggtcatccctaacaaacagcccactgccatgctgctatggtgacattcgttactacattgatttctgcagaccttcatccataatacgatggtaacgtctcgcttactatgcacggtgtgcccctgcctatatcttcacgatataccaagtggagaaccgtaggcatgtagtcattcaggtggccactctccttcacattatgtttagaggtcatgaataaccctaatcgtgtgacctcaaacagcatcgtattccgaataagtaacaagtaggggtgtttcaagttgcatgacacaataggatatgattctcaaccaaacttggcaataaacgcataggtttagcagtactaacaagccattatgtttaatatagagcatggcttactctgtcatgttcaaggtggctaaacccaacgcgttaatacactcatcggttacagtgtttttagaagagcaattgatatctcttcaggtgatacctggttcattatcctaattcagttggttcaggaagccttataactaccaattcgatatttttaagcatatagattaggtgataccacaccgtaggaaattgtgcagaatttggtgtctagaaatttaacattaagtgatcagaaaattctctgtgttaaacgactgttgcgaatctgtgtctttcaacctcaagtacgatctc

SEQ ID NO: 16 - DNA, 2000 bp, synthetic construct
  misc_feature 1..2000: note = Description of Artificial Sequence: Synthetic polynucleotide
  source 1..2000: mol_type = other DNA
taacaacctgtaactgtcaactaatgacctccttaccaaaattgagggtagttggttcaaagagaatgcagcatgacgcagagcttgtagtcacatcgttcttctagtacgcagagtgtagagttaagattattaaactcagagcacgttgtggacaaaccaataccagtccattcaattacatggtatctaacagtatcgtacaactttaatatggtctagggctagtgaagtgtaccaactacttgatacgcagtaaataatttcatcctatctttacgtcgccatcgaaaagcaaagttatggcgcgtggaaattcagatgaaccataaccaaacagataaattggcagcagttttttgtagacatttatataagaagagctcgaggcgtaggttaattctatacaacgctatgatagtcaagttctacttgaccaactacgctgggaatgtttattaaattcaactgggggcaaactagcatatactgtctgagtgtccttcgatggttctatacaaacggggtgtcgaggtactagtggaatggagaaactaccgacaaacgcatatcttatcttctactcgggatttatgaaattttttgcgtatactattcctgtgagcaatgttcaacagcgtagtgagcctcataacgtcacatcaattgtttcacgtctgtggctatcgagtattccttaacttaactagagtatagacattagagtctaattctatgcaagttagataactactactactgtcgtacttcattcagttcctgctcgtactcggcgacgctataaccggcctagtttgtgcgtcgccagataactgttccttttaaacgtataaaaagtacgaaagattaacccagcggaagttgggccccataaatgtcatatagggactcagactactgttaaaaactcctagtatacattgtagataatcaactaaagttggactatcaagaatcaaactgtaatcaggtcacagaacaaatggactaatagagctatctaatcatcatacagatttatacccagtggaaacaaaactttaccccttgaggatttactggagttgtgtcaagttagaaatcggtcaacataaattagaaaatgccttggaacgctgtataactgatcacatatagctgtgcctaatgcttcaatcgtcaatgctgaccacaatctacctgacttggaaatccgctacacccatatccatatacttaaagaatccgtactttatatcctattcaccgatgtccgatgtggcgctatgtgtgtctagtagtatatcagttcaaggcgagaatgaagaagaatacagggtctctttagagcactgtgtcactgtttcttaggccagttaattctagaaatcaaataaatgaataactcgcgacggctcaaaagaaatctatggtttacgcataagctgtaggtacttctaagcttgatttgcttccgggggatcctaatctaaatgtgaaggggcagatttagatctctgctcattgagtgggaggttggacattgaacatagaactaccttccctgcgtgctgtaagattatgagaatctatgctcggtcgttgtctaaaaatcagactacaagggtaagaataataacagaccgaaatagatgtctccttcaagatagtcagtttgcgcaagtctggcaggaacgttaagtaatcctgagttataatagcgcccttttaagctttcctggcgaaaaccgaaccaagcccccgtaacacaatgtcactatccgtacgaaagttagtgtaataacgactgtacctattataagcacatttggttggctatcttctccctagattcctggcggaaaagaagcatgtctacgttcgataggactcatttttgaggaaaactattataacggctataacgcgcgattaatccctgtcggtccatcattcacgtgagtgtaaaattgtgattagtacttaaacgggttcgtgga

SEQ ID NO: 17 - DNA, 2000 bp, synthetic construct
  misc_feature 1..2000: note = Description of Artificial Sequence: Synthetic polynucleotide
  source 1..2000: mol_type = other DNA
ttgacgatttatatagctactacttagccttactacatattccggcgtgccggtagatatgactaagttaatacttacagacattcaatattaggatttcggtgacctcgatctctcttgattgaataaaaaatggatattaatgcgtcgatagttgtgataagttatgtatgatgtcctgagggacatatgataatcttctaatagttaccttaaaccgaattgtgtttatgatgaaaaatataggtgaagttagcacctatcaccagactttgggatagttagtccgtaccaagcagcagttcaactgacaggaacgtcaattctgtctctcattactttggccatggattgaaaatcgacttcagtctgactcacaacagttatagaaggattttggctcaccactcttcgaaataggtcatttaatgcgtactgctttttttgacggccctttattcattctattgagggaatccctaactttagccacacgcaaactggtttatatggatactctcaagattgtttacatatccagaagcttatacttcctcaatgtgatgcacacaaggtgggatcatcttgtttctacaatgcagaatgaattaaaaatcgcccttcctggcacatcttgctgtacggctacagagtaaaattagctcgttatttatgagtgtttacacaacccaaatctaagtcgaatgtactttaaacttggcgtggattcatagacatgcaatcagtgttaaattgtcactcaaacacgtgcctgacttcagacaaattcatggattcaagctgctaatattcacaatagacgagataggggcgtagctttttctgtacgatgggggaatatacgagcatttctatgaaccaaaacaggcaaaatgagcaaataccttgtgcatcatatagtttccatcaactggagaaagcctcttgatcggctacaacttttcaagtccttgcggcgttggccctgaagtactatagccttttgttctcactaatctagccaatcacttgttgactattcttgcctcacccatagagtggtaatggaattccaaaaacctattcccgagtttaacccgtattgtttgagaggagttcctagtgtcttcattaaattgcacatggactctacggaaattactttttattaaatcatagaatctctgtcatcagtccatgcgtcctcagtcaataacggtcgccgtgtctacggaaaggttcattctatgcctgtaaagtacatctaacacaatttagtgtgggtcttctactacagttcacccgggaaacgttttatgtacgagtgttggtaaagcgtcctcatcaagtcgatccattgtaaggaatcgactatatactccagcttaactaggaccccgttacatcttaatggtaggtctaagaggtgataagactggaacctacatcatgagttgagtgagcaatgagagccagcaaatggtgggaagactagaccaacacaggatctcatgcttcctgtagcagtgcaactcagttcgctgcgaaaataattaacatatcccctattggcaaaaccctgcatacgtatttagcaaatatctgtaggggtcgtccaatagcagtgccgttttataaattgggttgatacataacactgaatcaagtgaaatcgaacggtggtaaaatggcttgaaaggggaagttgtttaacattcgctagcgacacatgttgcatggttagggttgctatttcgcctcattctcgttacgacattctcaaccagtagcccaccaacccaattaaggtcacgcacgaacctatcatccacttacctcttacaacataaaatagtcaatacaccttcctcaattagccttaatcaaataaagctagttatttttgtctcctggggatcagggcgcttacttcgtactcgcttcccccgctaggaaggccactggttcccgaagaaacgtgaataattgcacatgcttta

SEQ ID NO: 18 - DNA, 2000 bp, synthetic construct
  misc_feature 1..2000: note = Description of Artificial Sequence: Synthetic polynucleotide
  source 1..2000: mol_type = other DNA
agtatggaaggtgcctcggtaattacggaaagagcttatctgccggaaacttttattttgtttcatcaaaaggttatacgataataccgcatctaccttttcgtatcaaaattggtccacaaatccaacttattgtcatcttgaatcacacattcatctttccgtctaatgaaggagcgtcattacttgttgtatgaaacgcaaattctctacactagtaagtgagacattaactacagcctattaaataattcaggtagactgatgagtaatatttcttctatatatatgtgatactcactctctactgagttgactagtggactctttgttcttgtacacacacaacagagaaatgcctagaacaaagtcaaagaaagcgcctagatgactttgtaaattgcaccagatctgaagtcgagtcgtgaatagaactttgcataagactctaggacttccgatggcgtattatacttaggaaaccaagccggtagtaagaatcgaggataatactctgggaagtcttccgtatttgcgtcaacaaccagcttctggatcaagcatttcttaactagattaagcttcctctttcgttttaaagcgttttacttcagcaattgtaatccctacatttgtattagccgaatagaacgatgctcctacaacaccaggccgacctcatgttacgatggccgagaccataactcttcgatgaatcattagtggaagagttatctactgacggcatgatcctgggacatgaaattggaaagcatttgcacacgttaattcgccttttacttcaacgctcggacccggtataagataaaattagaccgttatcttcgtagatcgtaatacgtatcatctcgtatatgccgcttgtattcaacggtttcctttttagactggagcgatctacgctggcttggtttaaggactatgctagggtttgtacgtaatccctttaataattaacgaccgagctgacaaactgaataagtacagcatcaacaggacggttcgattgacagctggaaacctattaggcatcttggcccttagcataagtcccagtattatttgttcctccagtaaaaatctccccggaattagagcagcggtgaaatttatggacttgacctttttggtttagtcgtagagggacaaatatcatctcatctgaacgctcatcaccagttagttcatccaaattcaattaggaggcgtcatattgtcgggcgtctgtaacggagccagatctagaagttcattgctataaagaattagtgtgcttggcacatcacctaatcaaattttgggaagcagcatagctattcaggtgttggtcaaccagataaagtctatgaagaaaaaaacctgtgttagttctgcgtattagtattgtagtataatgtacgacatcccgaaagttaaattcaggtcgcagagtccctagtccaccgttctaactcacaaatcgatgttcggacatagctatttaacagtccatatttaccttaagtgtttcgacttatgtatgctagttaggtgtgtggctcgccttcccactgttagaccacatctagacggacatcgttaataatatctgatatacacaaaaacgtttaccatagaaaacactatattcatggacactttatcatattcctcgcccatcctcacgacccagataatagggagttgtagtttttctaaacggttttaatatgcaggtccataaagcatgcagtacattactgtttaaaactttaattcagatatatcctggagaagaaaatctcgattggttaatcacttcattgttaaattcgatttcgctatacgtttctgtactaggaaatttttcatattaggcacgcggtgttggttccgtaacactattaatttcctcccggttcgatcatggcttgcggtaagtcctcaatttaacataattgagataccgaaatcaacccagcgtcgcagtattttgagtt

SEQ ID NO: 19 - DNA, 2000 bp, synthetic construct
  misc_feature 1..2000: note = Description of Artificial Sequence: Synthetic polynucleotide
  source 1..2000: mol_type = other DNA
ggttaaatgcatgctcacgcctcgccaggttgttaaaccatgtacttactcaatttgacaactgatgtccactctccacctcgcgcgatgctactttcttaatactaacgccaccttgtcaaacacctagatcgttctaagtgtagcaccagacagagtagacaccgtaaaaggtgaaaaggggattaatttctcctccttttgcacaaaaaagttaaggggtaggccggaggaaggttaacgcgaagcacctgcgtaatcggtttcgtgctatatcggagatataccgtaatgactcgtcgacgaaagtcgaaggctttaagctccatgccccatgttggtgcgttaggactttggtaaagtggtaaaatttagatctctttgtgtcctttatatcaagttagtgtgaatgctgagttttctcattttttaatgtaagtgattaatatgaagatgtgtagtctaatttggagaaccaacttaaaacggaataggatcggtgtatcaatgcatatgaaaccggtaaatttagtcctgttgacctgaaactgatgggaacaaaccctcaaacgctatcgcaacaccgtcctaggttccatgcactattaacctgttattgcccgtgcggagatctggtttttattgttttatactctagatatattagcggttatgtttttctgttaatttaagatgcatagtctactttgacctccggcaacgtgatttgtagaaaatatttcccacacacactatatgtgctactcaggttacccatagtttatgtaataagtatcactttaaaccctccacccgcccatacaatagaagcccttcaattatacgaggaggtattgacctgactagtttaccaaagccaaagatacctggacaagttggacaaatactaaaggactactgtagcatagtgtttgcgggccagtatacgcttatttaaacgatactactgataagaaacactggggtcaacgtgctttcatcacctgtccattactccaacagtcccaattttttaaagaaggaattttcgggacagtgaacgcggaatcgctaataatattcagatagatagctcgacacaatataactaatcagacaaaaactattcaaaactttctcctaggtagtgcgcggctcttttacgtggggtttattcacctgcgaattatcctgatgcccaggagcaaactcattataataccaccaggtgacagcctacaagtttcatggcatggctgcaacctgcacacgaacgcttatgcagcatgtgctcttgagttataccagctacttgattcgatatatggtttttgtgaagaatttgataccattgacacgggatgttgcaaatatttaataagtccatgcatactaataccaacgccagagatagattgtcagtagaactcttgaagtcaatatggaccgagtgacttgggtggtttatcccactgttagaaagttatcgtaaaataaattcttggtcaaatctaatccttataaacactctgttattactctgcttcgaatatgttgttattgaccatgctgataactacatcctttatgttaattcaaggcattctctgaaagtcaacaattaacttcatatcagacatttgacctattcctcacttttctataacatgacaatcacggtgattaaaaacatgacgcgtatcggcagcaaaccactgtactgatatgtaagagcgcccgtcgcatagatattttagactctgtccaaatcactctacgccaacttgaggtcagaatgcataccgtggtaagctgaatagttcttatacactttctaatttacccagatgacgattttttgttatatgaatgacgatcttggcattatactgccaagactgcaatcaaatcctaaattcataatttagtaagtcaatagcagatctgaatcccataaatgaattctatcgaagtacctacactatgtcacgtagaacaag

SEQ ID NO: 20 - DNA, 2000 bp, synthetic construct
  misc_feature 1..2000: note = Description of Artificial Sequence: Synthetic polynucleotide
  source 1..2000: mol_type = other DNA
ctaggtaaattcttaggtagccgagttgaactattaataagtctcgtctgtgagtatgtcttccgttaggtattttcatatagcttcatgtgcctgtaaagacagaagtataattggatacatcagactttttatcccttttacagtctagaaagacctacttgaaacatgtttcttaatgggtaacgtagtgaattatgctcgtttttcctttggtagaatgatatttatctccatatgctctgagttggataatttgtaaagaattatacacgttaattcaacctctttatcaatgaactacgcgggcttgatcagagtaaactcacaatagtatcttgatcttcacaatctgatggatattgatgcgagttatacgacctgtggcatatcaacaatgaagtgaagtgtctgtccttatgattcgaaacaaaataagtgtccttgctagctacacccacaccgcggtgtgcatcccataaaggctcaggtatagtcttgtcataagcgctacactgccattcgtttagaatcattgtttagcaatctcaaaagtaataacatccgactttcgaataggttcagtttcctgatctactggagcctatatatatgcacagacgaatctcgtacatggcataagcaagtcatgagaagaggctgtaccacgtaaatataagcctctgattacgctgaagcttaataatcatcacccatctacgaatccgattgagggcataggctttcatgtctttttcgctgtaggtctatgcgattgtgagactattgagttttccacaatatggtggtaggtactgagtagggtacatttcactgtcctattgcgctgtcgtatgtctatccgccgttgccgtcgtcgatgttataccatttgactaacagtgttatgagtcactcccttggatgcgatgtaccttctgttgtgagggatgtaagttgcagttaagcactattagcgaataacgctaggattctggaagaagaaaacacagggtcgcttcaggtctcgagaatcttacggttagaaaatttggatctgaataaagagatgtctagccagtgtgggggttgaataagctaaatgtctgcaatgtgtatgcttctgcacagatattaacaaatccgccatatttaggcacatttggtaatggctgacaatcggatctcaagaattctatactgagttatcggactacaactaaaaagatgctatataaaattgtcataattcatgaaaagccagtaggccgaccatcatcgctctaagttgagttgtttgacgcgaggcaacattacgtgcatggacgatatacacgttactagttgtatggtatttcggctaagtttcctagctaatttcattaaaagctgcgcattggtgtttttcagcctatatactgacgtagtaaacttacatacttaattatactaggtaatgatatagaaaatggctgtacatcctttctgaaatgcttccatgcaatggtgctacaagtcttagatttacattataatcggaaaaacatcaacagtatgattacctaggaggagctagcatatccagaaagtagaatagcagaagccaccaacagactgggtgagagtgacgttatgacggatggatcataccccatcttaggagggtcaggtcatttctcaatcatatgtttccagatgcgatgcaaagacaaggcccagaaatttcaattgtaggccaatcgtccggtcgtattaatctcaaccaagtaaataaaaagcatgtgggctgggcgcagtcaaagtcgcttttcttggtccttactaatctgaagaatatacagtaaacagaggatagtggggctagttcagagtaataggcaacaaaccctttcatgcattactgtagaatttgatactattgcgtgtatcgcttttaactttataaagagtcgatacagcgcaggctcataatgtttggagtctgtctaataaacatctaa

SEQ ID NO: 21 - DNA, 2000 bp, synthetic construct
  misc_feature 1..2000: note = Description of Artificial Sequence: Synthetic polynucleotide
  source 1..2000: mol_type = other DNA
tttatctatttcatatattgctagataaagttgactgacttattacgattattgtcccagacagccgagctgggccgtgcgtcaatgcacgggctcagcctcctaatgttagcatttgttactcttggaacatttggatatagttgattttttgatagtgcaaaggttctcggtcatccggtataacgatactccctaccctagacattcaatcggtgcgatggtaagtccgtttcgcactgaaagcctgtagagtctatttgatgtttacttaatgcgatttgactcaaatgtaggttaggagtccgttcgcatcctatgcagtgataaactatctagtgtgtttaaaaagacgcaaccactaccaatcagaccagcaaatttacatcaatttatgtcaaaacgcccttacttcgtctaaatatagatatatcaccacatcaagcctgcacttctcacactatgttctatgtcatgtcgttgtaccgaacaattgatatttaaccggagttgaagatcagctaaagagagaagttatataaccaacaaatacagcccacccatcaatgatcgtgaaaacaaactgtacttaacagttcaagaacagtcaccatttctcgacgtacaaaagattcttccattatggttcgatacaaattgttcaaacgcctgtctatagcagggctccgccatatttcgagcatactaaatcattgggtggtcaaacagtctcacaaacaggtctgttgcgattcatacgagacgaccatacttaggcgttgaaatgtcgttgcatttaagtaacaaatactatagaccgctggtagtcgccatataactctggctccagattatacatgacctgtttagaaaggcaatgggaagagggcaaaaccccaagattgttcctaatagttgtagataaatggatgatatctgcatcatcactgtttagagaatcccgctttcctttattcggttatactcaccgtttctcggcgggttgagacatgcataacttctatctatcgttgagaattatcaacttcaattcccgagactgtcattatctatagttgaggaaccttcgtcgctgctattgaatagtaagaacccctctagtccagctgatgcttgtggtaactgcactagtaattcatctgccatccgtgcttaattgggcatgctttgttgcatcccactcccgaacttgaaggttggaactctcgttttgccagcacagttaacagggagtaagacctattggtgtgacataacagttaggtaaatccatctaaacacgtgtgtttactaatattcagtcggtggactaacagacaggagcttacccatccgtggatgttttcttaagggtgtcgttagaatgaatagtacatgtatagtactgtccgaggtgtagatagaataaatgtgaccgtgatctcagatttatggttcaaacgttctaattttccgaggagtagtacatgttggtaccttttcacattatggtgctaattaggcatgtataatatcatatcatagctttgcccatactgactatactaaaattgctattttggaaagtttataaggccgtttctcattgtatctaagacctaagcttcgcgtcaagaaatacccttacaatcggcctatttaaaattattcatttgtctagggcgcgatgatcctttccgaatattttatcgattactacttatggatacccgttagacgcttatcctcctactacaccgtactaattacgtacttttttcgaagtacgatctgattagtgtcgaccaccttgcccttaaatctgatcgctcccaccagtacgcaggacacacgtaacggtttcgatacccagcgagatcagccttaccagtgcttgtgtggtataaccacactatttcaatgcacaatgacaagagtactatgttaattcacatgcctatctagttcaattacgttcagactcataaaatgccattgct

SEQ ID NO: 22 - DNA, 2000 bp, synthetic construct
  misc_feature 1..2000: note = Description of Artificial Sequence: Synthetic polynucleotide
  source 1..2000: mol_type = other DNA
tcgaaattggatcgacggagctaatacgcaaattatttgtttgtgatttctatcgcgcttcaaaacctacaaaaaataacagcctttggtgtaattcgtcgtggccataaatatggcttattctatatatccgaggcccaggccataacaaattctccaagatttactaaattagtacggccttcattccgacgggaagtttaaactcaagccatggagtccggtagtctttcaactttgtcgtatgacggtatgctacatgcccccaatccgctattgaacaatggcaaacactacagcagttagccagagaattacgctctttcactttcctagaagtacacaagtcctgaacctaccaactgactgtacacaccctctatggtacttttgctgtttagttgccgaatgatgcatcatgtctgatttttcgggctagccttagctgagtgtcagcttcaccctgataagacaggagtcagaaacggaatttcattaataccgcctaaggcgaaagagaggctgtcatgtaagccggcaggtttcccccttacggggcccacactctcccctcgctatgaaatgacacttcacaaacagtcgctactcaggatttattccaagttccaacgatgttgagtacattgagaatgtattatattaagctaataggcagttttctccaactatcgattattcggctgatatagcccccatcctgagacgttattacgtcactgaggatgatctattcacacaacacttgggttaccatagttcggaatgcgatctaacgtctcacaatggtttttggtggaagtatagtcttattccccgggctatcgcaagcacccaggagtagtttcgttggtgtcatgcttatccctacgacccaccagagtgtccaatcaatttacacctaaactggaacctaatatattaatcaaactttaaatctctatatattcagactactttactcactttgatgttagatgcgtaacaagcatataaacccgtttgtgatcgtactcaatcgcacccttctcgttattgattgatccttgcgcgaggtaacctgggtaatctctaagttatcgatgcaccgtatcaacattcatgatcgaaaaaagtttagtgagaaggagttaatggatcgttccgactaaactaatggaattatgtatgggatgtatttcgtttgagccaattaactaggaactaactcatacatcttgcaatagtggtagcgtaaaatggttgaacgtagttgaaatagtagggatacgacatgtcccctaagcctcacccttggtagttctcgtaagcggacaacgcgttatcatcacgctttggagtgtactagtttatgtctactgcgttcgctgacaataagaacagcaatatcccaattctcagtactgacgtaggaccattagcgctataaaaaaagtagcgtgaactgtcatttattaagcattccattttatccagtgtccgctaggcggctaaattatacaaacagaacggtgttcttatactgttactacctccacaagtgggatttacgaacgcagaaagagataagctcactctcgctatgtgcaccgatgagtcatacagaggtcatcagtaaaggaactcaatctagagttacagtccagcaatccaatccggatgccaacaggcgtaacgattatattcaaccactaagccgcataaagtatcgatgattagcgggggaatacctcctaaacagtttgaccggaacgtctacaatactttgccggttatcaatgaaatatgcggggacgaaccatgcatcgttactcagcctttggtgtacgccagtaggagtactacttgttcttcttacacgacacgtagctacttctatgtatagtaatgtagttgactatagaatgacgaatagagaagggaaccagagctcacttattccgtcaactcgatttatcatgttgttaaaaaagataaaatgt

SEQ ID NO: 23 - DNA, 2000 bp, synthetic construct
  misc_feature 1..2000: note = Description of Artificial Sequence: Synthetic polynucleotide
  source 1..2000: mol_type = other DNA
gctactattttagatatgcatcaaagaaaaacaaggacatctcctgtatacgtataggtaataagaagaggatccaacggaaaaagccaccggtggagataataactattgttagcaagtccagttttctgtcaggggcaacgttaagatagaggccagggtaattatttaactactagctgcacttcgacttcattttctgagctctgtaaataccaatggagcgagtagctacggttaaacagatatcggctggatgtcggtggtaggaaaatgtgcctgttgcggctgataagcattaacttacctaaacatagattgttggttttcctaaggttttataagaacgtatataaagatttcttaaatgacaagcttagcctgcataggctacatgtgagtgtggatggcttcgacagtgatcccgcagtggaccagattccattacctgaatgaaaacgttcaattaaaccacttaccgtatcactctgtccttgtagccctgtaaaatgagacttgcggataccaaattagccaaattattcatctaactataatacttcttccatgaaacattaatacggccaccgggaagccaccgattctgtcgccttatattttttgctctatgtctttcttttagtccgacaactaatgtgaacaaatttcgacctaacaaaatagagacaaataaccctatattaatacaacgctacgaagatcttcaataggattggtccgattatagaccaattatacttttacataatatgtacaaaacatctcggcattcgatggcattggcgtggatattcgattgtaaaagcaatggatttttcttgcgctgaaaatgatgatcgccctcgatcatctgtatagcacgggtcgaagtttcagaaatgatagttgctcaatttggttcacttcgaatttacgctgatgtcccaagcgacatgtccccgatcaacatggttgttggatatcaaaaagctgataaaaaatgtgaaaggacacgcctccaacgcgtaactgtttcacctacttccatttcgaggaactgggtcgatttaacgacatcaaagttgtttgctcagacagtcttcctatgaaaatgaaaagtgatctaggagtagaacccgatggctattaataaacacactcttactaaataatttggcgagcatcagagcgtaggtactcggaacctgattgccgttccgctttctatacactgtgaataacaaagtcattgaggtgacaaccttgccgcgtgcacggtctaaagcatgaaattttaaagcaacaatcaaatctctaacggcctatctcaagttacgcagctggcggtaggtgggttttcgcactgactctttaaccaagctgctgctaaaatactcttacctcactgttgatataatggtcgcgattacagataatcccgcacatctgtcaaatagaagatccagtaaagagtccaaatcagagagacccaataaagtaaccaaggcattaccgtttcacgaggtggactttcatgaaagcataagtatggcgtataatataatgttatttggaaaaaagatctccacaacctgttttaccgctgaaaaacctaaataccgtaccagacgaaccacttgatagtcgaatgcgccattgaaggaaacattctccgttaatctgattttaagctcatcaggcttttatctttgcgttatctacatttgacgattaccaaggatcaattacgtgattggactatacttaatatcaatgtacgaaatcgtctacgatactacaaggtaaccactgataattcctcattgctctatgttcacactgaccttgctaatcgacgtggacttgcgtccttgtctagcttataatagtgagatttaatgacaatgctggtataataccgtgcaactacacgcatagaaattactcagcgctcgagaaaagtagattacttcgctccttcggagttttgcgtattttca

SEQ ID NO: 24 - DNA, 2000 bp, synthetic construct
  misc_feature 1..2000: note = Description of Artificial Sequence: Synthetic polynucleotide
  source 1..2000: mol_type = other DNA
cctcatttgcccttttatatttacccgagttagttcacgaatgtgccataattctggtcgcagcaaactgcggtgtttagaaataatcttccgttattcgtttatcaagacctcgttgtttagtagttctagctgaatgcggtctattaagttggagaagatctgggttcattacattagaacccaaactaattattaagttctgctcattagcattaggtagaatctattcttgtccggcgctgttgctactgggtttagtctaagtagtactttaactgttcctaagggatgctgcaaaatgagatatactcctccgataatgatcaatttggattttgggcagcggtaaatgttttatagtgtgaattgtgttactaaatttcatgacgtaagctgaccttctaaccgtcgtgcttggaggatttacgcggcgccaaaaagaaatatactagtcccaatcgcactaggatttgtttaaaaaaagacggaaaacctgcaaccaaaggtgtcttgtactgactctatctgcaaaatttggatgttctagctccgtttatggtcgctacatggaaacgctattggttaaagattcactataggccagttcaagtttcccgaaaaatcgtgacggacgttatactctaacattgataagaaccatgtatcaagcgatccgcaatatagggaaacacggcgaagatcaaatttatagatgggaggaagcacacacaatatgagtattagtgtgctgaaatcagcagcgtaaagtgcttctgttccacctatacttttacgagtctcgtaatagcgtattaccatgtaagatgcattaaagctataactttatggcaaaaaaggtaatttattcgctcattactattatttgtcgttttgcataaataaagtgttgttacttcaggaagctttaattctctgtctgccttaacccgaattctacgcgatctccgtatagcagatgagaaccggtgacacgagacccgcactcgcaagtcgtttcttgaggctaacgacaaaatgaagccatcagcgaaatctcatccgttaggctacccaaagttaagactttccctgtatcccgctaatgcgtcaattggtagacgtatcgggattagatattcaagaccaagtcaggtagagttggcgctagttgaacatggacctggccttacaaacaagaagaccacgagagccctagtacaggaatttatcggaaaaaataagaaaattaaaatccccgatctgtgtggtgctcaaataaggcaagggcgcttagcctcacagtcgttactaagtcaaggttctaaaagcacgtgttttagcttgatggatcatgacttcgctacggtcactactccaccgtgtttctggaggtatgcaagggaaaatcgagggatgtgctcaaatctgtggcaaccggagcaccattctaggtaacttccattaacttttgatttagagtatatggttaagctattaaacgtttcctaaggacaagtgggatagtgatatacttttttcggcgacatcaatccaggattatccgctaacagatcgcctagcgctaccgcatatgatgatatccttaggaagagatccaccccggccaagaaactccacactcaataggcggtgacctatttgtgagttatgcagatgtgtttcaagactcaacgccgacaaagttcaccaccagagagtgtaaggcttatcaaatttctgattttatcgacttataaatttgacacgtctaacagattcggcctttgattgtaaacatcgccgctatgatattttcgtgatcctttgggatacgagatgcatcagtactggccccgaatatttccattttaattactgtgtaatgcttaggttcacaatcaacaagtagttcgtgaaaatgttactataatatccacacaaagatttacgcactctaatggtggacgttggacctctgttaacccgctttcgttatt

SEQ ID NO: 25 - DNA, 2000 bp, synthetic construct
  misc_feature 1..2000: note = Description of Artificial Sequence: Synthetic polynucleotide
  source 1..2000: mol_type = other DNA
actaaagtcctggagagtatgcttggcctcgtgcggtaacatttgaacagcatgctaggtgctagtagaccctttcttgacagcggaatttgctgttattcaaaccacctgtcaggccaattctggagcgcaacccacagtgatagaagatagtcgttacaatcaaatcccacaacttgagactagccctcagactgcaacagtacacagttatgctgtggagacaaataaaatacgttatgtattggtcattagatttggctttcttatacgtcgtgtagtaatgcttgtgatcggttgccgacatggttacgaatagctgtttattaatttaaaattcaattctgtcgatttagaggatggataatatccgctatgtagacatgagtgagttccttatccttcaattcccttttttctgttattttggatctacgaatgaggtattaagttcgtagcactcgtccgtttcgtggaatgacttattcgagatggcttgataaggaattgtacctcaaaggtttcattgttaagaagatgaattttcacgcccatggcataagcatatgattacgtccactaggtcatagacacatgataactcgtcgctcaaaataatcgaaagaacgtctatcggccaaattattactttgatcccaaaggagaaatcatattggggcgcgggacttcatgtgtattaccatccagcaagcatttgataaaagtaactcctatattattatgaatagcggtaagtttctttgaccaacctgacaataacaccaagtgactcactgagcccgttatctactaggtattcgcgaataccgtaaaagcttgatgcaggtgacaatgagaattatcattagcgtactgtatgctcaacctagcctccttgcaagatttcgttctatctattttgtattcatttctttccgcgacatgcattcttttgctagatcctgggtcctgcaatcatttataagcacgcaacttagcttaaaagtgtggagacgagacgtacaatcactacttcccatcacttcttctcttataagcgtaccgaaagacctcgtattttattaaacaataacgtgcagttggcctaacataattcgatgtctttcagtgttctaggaaaggtgcggtgtgtctagcaagcatgtcagccctacagattcttaacatacctatgtgtctaaatcgagtatactataatgatgtaccataagcccttgccaaaggatcatattcggactagttattgccttctggatggggtacttagactaacattttaaacctcttgcgatacgacctggtgctaatacactattccttcttttctcacgcgaactttcagtatcgtacaaaagtatgggatttaaaccttttgaagtttggtcgtgattatttgtttttagggcctcctcgacgcctcaaatagggatttcttcagcactacatattttgagccgtatgcgaacccttcttaggaccgcggtagtttgttcacgagcacgttggccacaccccaattatccagaaagccggacttaagacatattgagtttgttagtgcataaatagggtcgcatattgatctgcgactcgagtaaatgtcgtactggtgatatattctcccgttttcgaaggccccaatcaattactaattaccctatttacgaatgtcgagagatgttcaaacgaaacatgagggcgcatcccaacgcccattttgaaacttgattgttgtataattcttaatttttgtagattcagcgttcttgacacattttaaagacgtcagttcaccgtacctaccccttcggttacgcgaaaaagattaggttaacgatttctatcgttcgttggttgttatttctgcagtacattaattttataacttgatatatcaaatctgtttttgattaatgtttgaaagcaaatcgtaacaccaaggaatgcaaataatcatacgtggcggaccagctactata

SEQ ID NO: 26 - DNA, 2000 bp, synthetic construct
  misc_feature 1..2000: note = Description of Artificial Sequence: Synthetic polynucleotide
  source 1..2000: mol_type = other DNA
tacatccccatcagtcaagacgattcgttaacaaatatcgctgactgggagaatcccagcatgtcttggctggctaaatagaagctactatgttacgcacttccattttgaattacaggcgacaacattaccagacttagttaattattaaacaagatcactttgcgacagtcctctgaggatcagttagagtgcaatcacttaagtaatacaaaaatacagaaggattctctggcgaacaggtttattagcgcatggccaaatttctaatcaacccctttagttagtacccatttctagccaatatcaaatgtactccaagccggcgtatagttgtcagtgtgtgatttaacgaataggatccccccccataacaaatactaataagagtggagcaattatagtttagatcgtaaaggtttaaataaataaacgtcaagcacaattatggactcgtatggggacaaattgagcctactagcagttctagcgaaataagttgacctaaccagtccatggactgccggttcgttgaagtcggtccaacggattgcagatcattgctaggcagttggtagataaatttctagtacttatagtcacgtaattgtcaaaagtcctacgagcgtggtcaccgtattactacgacctccatagttttctaccgtgcattctgaaagaaatatggctggagtgtcctagctcatgatagaaaacgcctacacttagccaatcagacattaatgcggtaacggatcaagcattacagggcggattggtcgcatatcattgcacggaaagcgttgccttaagttcggtacattccactttcaacttcatattgactcaaatagtgggacagtgatttacgcggagttttaatctaaaaattcttgagtttatgatagaacagatctaaattacggtttttatatgtagtggtattaataatgttcataaccctagatatttccgagattagcactcgttcggcgcattgccggtatagaacaatatgtgaagaaatttgcacctaagaagttgatattctcctctacatgcgtataatatatagtaccataagtggatcattattaaaataaatctgagtgggtggacttatcttctgtcaccctaactggatcagcagtgggctagtagccattaaggaacaaccacttggcccgaaactatttgaaaagtgataaatacatacacgatttactacataaccactcctcttgttgataggcatgcccaaggattcgtatgggcgattttccataaacctacagggtgattcgcgcatataaataacaccaaagcagtcaggctttttgtatgaagtgtagcttccctaacagtatgatagttgtgtagagtcgcttctgaactggctgaccctagttataattagttcggcggaggatgggccgcgagacaaagtatactcgaaccttagggccgcattccaaaggttatttagataaaagtacgcaaacccgcacatgagttgaaataatgaagtacaatgttatttattgtgcgtggtaatagtctcgtgactgaaaatttttacctttagggttctctatccggaggagcgtcatgagctcaaatacaaaatcggagcattgactcaattactactttatgacaaattctacgtctaagcgatttttctaaatcgccgtgatcaacaaactagatctacaccagtgatgcatgctcacggcgaatgtcctgaagtcagatctaattcttaagggttggattagctggctatagcaagccatattaatatgattagtcgtgtatggtttacgctacctctccatagatatttctaacttacatttgtaaatgtttccaagcataccgtcagtataaatacccaatgatgtggctctccttcaagtgtttagataatagctatttccataaggtgcctcccctatccgctcatcctcgggtttcatatgttgtaagtggcacttaga

SEQ ID NO: 27 - DNA, 2000 bp, synthetic construct
  misc_feature 1..2000: note = Description of Artificial Sequence: Synthetic polynucleotide
  source 1..2000: mol_type = other DNA
ttgtttcttggagggttacttacgattattcaatgtcaagctggtaccaaataatatgttaacatcgacaaccttgctgattctttaactgtacgatttactcaatccttacaacagtctttccccccgatgcttccgataatccggatggaatgtaaaagctttaatttagccataatggagctactctgcaacagtaaggcaaaattttcttaaatggaggccaggcaagatttgtccccgccagaatagcctactccacaatattctctttaaatattcgccatgctatctcacgcatccatgaacaggttatgaaagcgtagagtcaaacgtacactttaggttaggtgccttgtggggatttcacgccacaaagtagagtagaagcagtgtatcaaactatgtgtaaaagtaatttcatatagtaatagccaccaagaatgcgaacataggtgtcggcctgaagatctaaaattatacttattaacaatcatgtgagtaggttggattttaacacgttcataagtatcgatcgcttcgcttaaatagaataaagtacacatcatgtgacgacgcgcttcgattattgtgctgcgttaagagtagtaggataatttttgatagacctgtctataacacggtatttaatccgaagttcactatacaatcataataggatatcgtgttctgtctcgatgatctattcgtcgcttcgggtgcaatataggattcctatatgaaactcacttccctgagcattgggatttcttgatagctagatcgcgttagagtcgggcggtgtatagtctcggatacaagaacataagagtaattatgtggaaccttttcatgtgattgtgctaactgtgtgatattcgcaataattcctacatcttagtttttagactggacttttttttcccaagctctaagcatacattattcgctgcgtatgtcactgacctagaggaataagtgttctgctgtcaaaactaactctctctagcagcctttttgaccatattatcaattacgcgccatcccataataacttcaaaatttgcaaccatcggaattagaaatcccgacgtaatcaagacgaatcttcgccgattatcgagcttacataatcgaaggtgcatttctgaaccttggctacgctaaccctctagtcggggcaagatgacttggttatctggttaactaggaactcctagcctcatattgtatcaatctgatctaatacagcgtctaccaattatttgattaggtttgcttgccctcatagcatcgcagcgagtatctcacaatgtgtatgggtattcttctagttacgagtttagacggagaataagccgcttgtggttaacctctgtaaatacctctagttgaataagtgtgcaacccaattcacattcgtcatgttaacaaatcggcaatctttccactaatgagaaaaaacaaatcattaatatatgtgaaagtaattattgtgtcctcataacggtaaagacttacgagtaggtaacaatctcaacttcaccaattaccacctagattccagcaccgccaacgtaatcagtgttccgtgcgtcttacacaagagaactccttaagcggctagcgtatacttttaagagcagtgggtatgtggcccggggcatctattgtttaccgtaatataagcgcactagtctatttttacactaaatatcattccatatccggttctttcagtaacaaaagtaaacacagtgttttggaagcagtgtatcaagaattgtgaacttctttcaccggcgcagggatccactgtctagagagaatcttaattctatcaaccgaccctccatgtcttatagattgtgtcaacggagcacctaaccgtatccttaaaaatttagaggaaatagaactctcattcttcagcctgttaagccaattaaatcgaaaccgttgctattaggtgtaacggtagatgtgataaaagggtcaca

SEQ ID NO: 28 - DNA, 2000 bp, synthetic construct
  misc_feature 1..2000: note = Description of Artificial Sequence: Synthetic polynucleotide
  source 1..2000: mol_type = other DNA
aggacgagctctaggggtgcccctgctgttgttggttatttaaaagccgcgatgaagagaacgctagggggaaaaaacgatttgcctagaatagtggatcggcgttttgatgtaagtgtaattgggtagaaggacttgttttacatttgcgaaatcttgctcggggacgttataatatggccttgaaatggatgatgacaatatagttttaatgttattataattagagtatcgtattattaaaaaggatgtccactgtggatccaagttaagcattaggcgcgttgaagagattgtaccgcccgaaccaatgcaattgacatgcctaactagcaagacaaacgtgttaagactaaagtccctcctatcaacgtacacctcatacgcttgactaggtagaatactaaaatactctcgtaatgaatacctattatctaagtgactgctgcgttcttttaggtggtgaactggctccggaaagtgtgctaatagtctatatgtccgcgcctgccacgtaaccacgaggcggatcagctagaaacataaagccgtttgagcaataagtgactatacttaacggtctgtaaattcgcgcttcaatacctcttactctctgcgttctatcccgtctttttataaattcaactatacgctccattggttatcgccatatgagtccttatctacttaaactggctaccaattccttgctctaagctaatgaaagtccattcgcaggattacaacatcaatgctaactttctcttgcatacagtatatcgtctaataaatgtataggctcccggaggtcggaacagcagtactcccggccacgtatcccgaatacaaaccttattagtaaaggaaacactagtgagagcgtacggggattactcgaaatatcgcaggaaggtggttaatatgccaaggaaatacgaataattctctccgcattccgaaactgttagcacatagacaagacaaagagtttactgacacatcttttgacaacccgcactctacaacgacctactctttatacaagtacggattattgtaacgctccagcctagagagagtaacccggagttatatggagtcgcttgaggagaaatattaaagctgaattctgttacgactagtaacattaccagccgaggtctgaataacgtgcctatggcgatcaggacaatacgagagaatttcttctaccacactatgtgcagcagctcactcaagagtcctatgtagactgtttaaccagtaaggattgttgtgcggaagtgtaatatggtcgagaaataccgctaatatggataagttaattgaacttcggacgtcacattctcctataatgaggatctattcaaatcgttttgaagtaacctcctcatttgagtaaactaggcttgcctggagatggggcccccaactgtaatgtgttatgtttagtttgaactcagttggctcaaagtatcccgcagtactaatattaaatcttgttattgtacagctggcgaagaaagttaagaaatgtgactcctatactattactggatttacaaagtaagcgtctttgacattaattatggtattgacaaatcaaatgagagacagtaagatgatgacattcgctcatattgtatggctcgttgactgatgcaaatagtaccaaaccctttttttagaattccagatgaggaattagatttttcagtcaatagttacttgttatgccacgtaggcttatgtcccctaaatcgcatataataagatagagtgcgaatgcgtgcacgtgtacactaatcagggcaaactaaacatttaacctttggagaaattccgtggcgctgaacttagtgatgatatatgattaagggatccgttttgttttcgataatctaagaactgacgaaggcactaatatcggagttacacaggaaatagaatgtcgcaagatgtgccttaggagtcagaaatcaacgagtgttgatcccaca

SEQ ID NO: 29 - DNA, 2000 bp, synthetic construct
  misc_feature 1..2000: note = Description of Artificial Sequence: Synthetic polynucleotide
  source 1..2000: mol_type = other DNA
acaacgactttcgaaggtggctgaagaaaaccacatgataaaatcgcgagtatggtaaaattagctacctgagtatatttaatcgaggttatatcttttgtgagtcggacacaaattctatatttgacggagcatagggcagacggacatataaaattataaacagtctgtacggcggggcctccaattggattcccgcgatcatatcagtcagttgggaaccataaattgcgaaactcagtactatgcttcaatgcccctttctaacacgtttatcgcttcaacctaacggtatttgcactccgactatcgtcttatgcctcacaatcagatgtaataatgcgggatttataaagattttgaaccattggacaactgacggcttctcatctcaccttgacgagagtatttcctattaacctgaatttcgctaaatacttatctttatcgccaataattcctttatgatacacagggcttctccaattcatccacgcagaaactgcccaaatgaggagaataaaaaactttataattaaatgaattttatagcctatgcgtatccccctacttcaaatctgtgcagtgatgataaactattgtaatgaagatcatttaattcgcgagattaaacagattcatgttctaatgcgattattctggtgtgatatcgtgcatggataatagaaagctgatccatttagaaaccaagcttatgcctatccgcacctttaacacacgcatagattagcgctctgcgcgaatcctgcgcgttgcaactgtactgatacaatgcgcaccaaaacaacttatactctagcaatgtacacacatattgcgagccaatctgttcagtttccctttgatatttcaggataatcagatggacgccaaatagattactcttatactgaggaaaatatgaagttcaggttcagcgttacacgcaaatcagcgattaggtctgcctaatatgatttacgtaaataaatctaccaactagaaatccggatattttacaataatcatggcaacgggtatgaccactgggttcgatccatatacctgatgggctcggcaaaagtctgtaagaattctctacatcccgatcgatgcttctttatttattttacttcataaactcgtatttaagctatgcattgccaacagggcttaaataagaaaaagtgttgcacacagaagttgctatgccgcaatggaaagagtactttcatgaaaatacgtagatatttaggagctttcatttagtaggtcatctggttgaccatatactaatcggatacttgcgaattattgtcctttcagcagtgaatcctgagactgataagccagcaggcgggaatcgtattagtaaaatttaaggacatctgagtacgggcgaaatctacaacacgacgaaatcatcaatctattatgacataagtattggacagtacgtctgactgggaaacatagctttatgttggatatgtacattagtgcaaatctgtgttacgtgttaaatcatcgcgttctagaactcttaatcacatagcgagctaccttggcgaacactcgttactgttctcgttttgctatcatgtcctaaaagcggcaaaagttattactgcaggaccgaaaaatatgaaaaacttattttttcatgggactacacaaatcgagttgagcctttaagcggttctatgttacttgagtatcttgaacttggaggggggttataatgataatagcaatacataggttatgataaactgtcctgttttagatacacgggagccttagtaggcttattttaatagtgtagttgttgatatgaataatatagaaaggccatggaggagaagtgctatgttaagagggcagtcgcggtcacgtgtgccattgacgctcacttatatgctgcgttttcgcagtgtctcaaagattaaattagccatatggtgtctattgttttcgtaaacgcctagcatgcgttcgtc

SEQ ID NO: 30 - DNA, 2000 bp, synthetic construct
  misc_feature 1..2000: note = Description of Artificial Sequence: Synthetic polynucleotide
  source 1..2000: mol_type = other DNA
cttgtgcgtcgaaatcgaaactcaaatagtatgtacgctgaaaataataaagcctagctaacaatccatccgcgtttagatcgtaattcacattttaccgataaaaagttaagtacaacattggaattgttattacttagccagccaataacgcgtcctaattaccaaaaaaaacagactctgaatcatggtagattaattgggtatcgataacattatccaaattcagggggccattcgcttaagaaaagagatgttaacgtactccagcgatctgcggtgttctgactgtaaaaatacgcatacatttcacccatagcagaagacgtaggacgtcttttctaccaggtgtctgtattacataccccatgcatatctaaaaggattctggacgtattttgatttttaccagttgagatagtgtcaaattctgactttcaaatgacaatcgcaaaaatgtatgcgaaggctgatgatcttgtaatcaatactggtgctagtcacatactgttgtagatacgccagatttacactatacacagtgaacaaggtcatgtcaataacaactatttttgtttataatcactaaccctgcatatgagggtcttgatccaagttcgaatggttgagaattccgagtttattggtaagggaagatgtatcaaatataatccttgcttacttcccaacagtcacaagaagcagagttaacgactgattacggctggaccaataaatattgaaacatcgcaataaaacttgaagaaatttgactacaaagtttaagtgtatacagtagatcggttagggtatactcaattagggcggaacccgcattcctgtcgataagctagtagtaggtggttttcaggttggtatcaaccatcaatattcgacatacattaatccagtgaataggggcgtccggattttgtaaagcattaaccttctgtataaatactgccaatcatatggcttgagtaaccgtttttgtcagtggaatcgtcccctcgctagaagcatctgtacgatatctaatggctgtagttgccttaaatcggaaaggtaagtcggaacctgggctctcattcgaataagaccaatcctaaacggcgaattcctttatcttgttaactgctgtgtcaagtcctcttatcgaaaattcttacatgtttactcttgcgattaactatggtgaactaatcccaacaatgactgttcgtaatagatgtgtttgtaaaattagtattttggtgacatctctagtcatttcatgccttcatagatcatcggtatttcgcaataatctgctcatactatgtacagaaataccactaccttctgacacccttgctagcactctggaactaaataactcatagacgaaaatacaatgcaaagctcatcttcttttgaatattgagcgaagtagattgttgacgttaagaaatgagtagtttcattcgagaacatccgtaatcaactacaattataatctcacaagatcggtctattaaatcgctcatactcctaggactagaaccaacgatcgaatttgtgctttgggcttaggtaaagacgtataatcctacctagaagttatccatttatccacttgataacatatgtctattccccaatcataataagacgtagaagaaaacgactctcacaacgacagtatgccctaatatgcgatggcgactgaaaatcttacggcgcccgcctcaatcacgttcacgtgacccagcacattagatccaggactgactcaagatcattactcggcgatcaacgcactatcctcaattggctatgtgcgaactcctcgtataggataaggatattccggtctccgtatacgctaggctcagtaacgcgtcttactctgggtcaagggtttaaagatcatagcggtatcatacaaaaaatcatatggcctactttgtcgttttaagcgaagatcaacgacgtaatagctaacttaatgagcaagattt

SEQ ID NO: 31 - DNA, 2000 bp, synthetic construct
  misc_feature 1..2000: note = Description of Artificial Sequence: Synthetic polynucleotide
  source 1..2000: mol_type = other DNA
tcgataggacagataagtgaccgcttgttgagtcttatatgtattggacttaacatcgagcaacagtctgtaacatatgtcactacgtgattgaaggccgtcgtcagtaattaaggataaggcggtaagacataagataccgtacaaggatatttatcgttatctcaaggtcaaatctaactataggtaacaattaccttctactagtaggggaattccgttggatagctagtaaaagattgcttcaactaatccaacaaagtattacatcaaaacagattggttatcaagattggagcttcagaactagagtggtgagcaaagcactctcatgccttttgtaagaaccgggaatgaaccgcaagaatcacttgacaaaggtattgggtggttatgttgccgggaagctacgattatatccaataggctacggtcgttgtacaaccggttgtctatctggtacttggttgatgacctaggtgcgagccattctgccaaatttatatggagattaagagtggtctttgcctgatgaaagggccaactgccgaagtactttggagcagtgttgactgcagctccaaacatcttgtattttaatatttcggaatagacatctatcgttagtgaggaaagaatttgatcccgcgctattttcccgacattctcaacacttggattacttaactcatagaattttctacctattatattataacaaaaaggtcagtattggtcctgacgtatctgattcacgtattacggggcggggtggaaaaacttggtttcctagagccttagacgagcgttaatatacaacaaactagtttcacataatattacgtatggagtagactcaaacaatggatcgcggcgacgtggatggtattatcgcatgatgcaattctaacgatgaatttgtgtccgcgctgttgtcgttttaacaacgattttgaggttatgatagttataatcattagaacatgtccgaaattcaagtggttcaccttagctttgtcaattttgtcacacttcagggagggtccaggaggaactgcaatcgtcagtctgaatcgttcgagcagtagaaatgacctaatttgctcgtgacgtactgacgataccaaatcaatgattgagttcgaggatctgatgtttggagcttgcgttggacgatctgatactcaaaagtcgacactcaacattttttgccacgacagatattctccagacttaagaaatccttgctgaatatcaaacatgcagcttagattagttattatgtaaattgtgagatactatgctaactcgatagtgaggtgttggtctgacaccgtgaattaataggtcgtccttaacaagtaccacttagattcctcgcttttgagtctttgacgcctttggccggatgcatgtataaatccttttcaaaaggctgttcattcccatccaagttctgtaataggtctatctttacttctggtaacaagagggagttgggttacgacgagtaattgttgtagcaaggataaactgctatttttgattaacagcctcacatataatacgggcagccaagtcagcctgccggcaaatttagcagtgtttctgctcgccaatgtctcgagactcctagctctctcgtccattgctgactagaactagccaattcggcgagcattagagtgctaaaaaaatcggtacaggagcctaagggtatccgggcagaagcaagtggtgccaaagacagttagtttatgagcttacgtccaatgatagaatttgcaaacggtatggttaccttcttttctgtatcttctcaatgtaatatgttaatgaacacattgttaatgtggtttcatatagtaaagtagaaaactagccgacaaccaaagtaagaggagcagttttagaatcaaatacaccaacttaaaaatttgcatctatgtttttgacaattgacatacgacataataaaagtaggatagttgtagatcgtc

SEQ ID NO: 32 - DNA, 2000 bp, synthetic construct
  misc_feature 1..2000: note = Description of Artificial Sequence: Synthetic polynucleotide
  source 1..2000: mol_type = other DNA
acaacaatccagaattaaagagtcaatgattaaagtctctataattcttggtggttaaggtgcaacttttgtcaagccaatgcttctctagcttacgaaaggaactagtattacaatttgttaccgcatatactaatgatcaaacattgtacaggtacggttaataggcgcactagtaacaccgtcaattattatcctcgtccgacctgagaaaggatgatagatcgtgcatagagggacttgtggaacgaagaacatttcctacgcagctacaaaagatatattgcaccagggacgtcacactaaagatgtatactacagcattgtttctcataacctctaggtaggtctgtagattcagcgtatatcgactacctacatctcgtctgatattcatctatcgccttaaaattgtgtaaaataatctgaggtcatcaatggttttgtttttacattatgtaaggtccgtaatggtaacttgtgaaccgacatagttccccgtcgcttaggtgtgcagataattagatccaatggatcaattctcggagatagtcttctacggcattctatctgtacacgtattggtacgggggtcgtaggcagggagacatctacaaaagttagcggttgctgaattattaatatacagctttacgcttatacggttgactacaaaaaaattacaagattcttcatgagattgtacctgtcaacttaattcgtatcaaaaattctaaagtgcgcatctaacttcatacaacggagaaaagtacatataagtagggtgtgaacgcagataacgttcaaaatgatttaaactatgattgagatgtccaagttaaggacggtagggttgctaccgtggactataaaccctaatgcctaaatctttatattcgggaattgtttcgggttagggggaatacgcacgaggctaacacaatatgcatagtgcgtatcattagcgtatggaggacgaaaagagatatacccaattatagcctgaatgtcttaatcagacccttatcgtcatctcatttttgactacaatcggtaataactactcgggtttactagatcctaacgggatgactcataatagaacgaatagtgtaaaagcaacctacgcgtaagaccttcccggtcatgaggatgtcatcctatgcaagcgttcctcccgcgaacgccacgtgatctctcgattccattctataggattcattaaagctctactattaccccaattgctgggtgttctaagatctataatgttattgtccagattaagttctcctgcactactcgcgattgtgtctttcgcccgcttgtccccccgtaattggatcgggccttcgcgttctgctaatatttgttacgtcacgtcggataacccctacttgtgcaacatcctgacgaatgttgtaaaaagtttttctttggaaatttgtacagttaaaagacaagataatatgattggatggcaagtgactgtaaagttctatccagtgtttcgtatacgattaatgaaactaaacgagaaactttgctgacctccacccaagatagccttcactctttcactaactccacggtgaattttttttagtaattttcataaaggcaaagactaagtttacctagtaacgccaatccccccaccatagtacactgtgattcgaaaaaaggatatttttgagcttctatgctttagggatatttagtttaacggaaagcaccgtcagcttggaatattaaacacgcacatgatttatggacccatagttgacatcaaggtctttgataccgacggttttcgtattttccagtgaaagccgaagctttacaaaggagagagtaattgagcaaatttctcactgcatgtcacagggactgataaattagtccaaaaactttattacgtttgaccttagaggtaccctaatgcggcttattatttggaggccagactattgcgcgtaacaggctgtttgagcatcggt

SEQ ID NO: 33 - DNA, 2000 bp, synthetic construct
  misc_feature 1..2000: note = Description of Artificial Sequence: Synthetic polynucleotide
  source 1..2000: mol_type = other DNA
ctcctcgagcttatagaaaagtcaacgaatgtgtagaaccaagaaagtgaccagctatcaaataaataacaagtgagaggtacagcgtatctaataggcgaaagtctagctccaggtatcggtgaagtctaactatgaattaaacgcattgcgtagctacatggttttacacgcaccattaacaggcgcataactactgcctgaatcgctctgatattaaagtcaaaggaagctaaagacttgctatatcgttgcatggtgttaagtaaatacgactcgagtattttaaaaaatcctctgaatcgaccaactatttattcgttcattctctgtcattgagtagcgctaatcaatgtagtatttggatcaataaccctctgggttaggcgactacatgagtacccttggaaaaactctggtcgagcaaaacaagacacatggggttaaataaagtctatacagtttataattatgcaaatttgacgaattttgtacagaattttatctataatcttacgggggtatacatatgacagctttccggtgttacaatactccttgtgctttgtacacttggcggaaaattcaccacaatgtatggggttccgcgcaagctctctttttcggtaatctgggattccttttttgtgcccttttacataacaagacgaattggtctcctttttactcagaaagaattataatacttttcttacttgtccgtttcccctcatctttttttacctccaaatccgattcatcgccttaagtccagtgtcttccaatgtagtggtttaacgcgagctacataaccatcccggatgtatacgattctacagcgtcttgaaaatattatgtttaggtttcgggtgaaacgcacctagaaattatagcaataataatcttaaatctcctcatcataatagataggttattgataggcgacatgaaacccagcggattcacctatcaccaatcaaaccacagttccttttgatgcagtcattcctacaggcatcctattaacaaacaagcgtgtgccgatgaagaattcgtatctgttaagcatccgacggcacatgtgcaagagtcgatctcctgataccaattttagtacttctcctctgattaaaacaacttccaaagttccaacagatggagtatagataatcaagtttccagaattaatcagtaatttgacaagtggaagcgctagaggactattcccggtaatactataacaagtaatagtgaccttgtgtataaatagacgttgatagatatatatacacttcttgatagctgaggtagacgttgatacaacccgcaagtgagtccattaccttaggccctacgaacatgctcaaacccttttatgctttcccagactcaaaatcaatacgtagatatattgtaaccgtatagaaaagagcttctgttggatacagtggtataacagctcatgttcaaggtttatacggtatgacaaatgtgattttcttttatgtgagataaccgaaccaatttcgaaagattactactagttgaaataccaattttaaaggtatcctttccattagaccccttatattattctactgtattagcaaattttagaaagttcgtgtggtactcaaatccgatgaaactattcaccgtgaccattaaataagtttgatgatcaccgagaattcacacctcgtaaataacacctatcttaatagaattcgtgcgcagctctaagagagagcatcttccaaaacgaagagctgtttacaattgctgccacgtctttgatatacactcttttattgtccaatccgatgtttcacaataggatccatggttccggttacttcctagctaaaagggtttgcccacgcggtgagggaagtctgtcggtatattagacgtagtgttcacgaataagtaagatttttaatttggaatggtttgcaacaattacataaggataagtaaacgcgccgtataatgctcta

SEQ ID NO: 34 - DNA, 2000 bp, synthetic construct
  misc_feature 1..2000: note = Description of Artificial Sequence: Synthetic polynucleotide
  source 1..2000: mol_type = other DNA
atctcatagataactctatgaggagttaacgcctagaaattttggtctgcatggtacagttacatatcgtatgaattcgtctaacatttgaacggaccacaccatctgatccgcactcaatggacagtaggcattcggttacactttcgtctggaagaacagtccgaatatgaaaatatgcttagatgattccaagttaatttcgtctataaataagtagcttttgctctataaagataacctcctacagtcgtaacagagctcatatacgataagaagagtatacttttagtttttcgcacatttagccattcaatcgagaacatagacgcctcgagccgaattgcttagcacattttcctaataaatgtattcgaatatccaaaatgaacttgcatgactccgtagcacgcactagatttagtgtgcctaaagattaatatcccaaggttgggctagaactaaaaacgctgttgccaataggttagattgtaaactggcccttaacaagctgattatcaggtgctttggatacttagcacatacttaacacatcggcgtgaataagtgggaaaatgtgcacaaactcattagaaattctgtgattgggtctttacgttatgttaaagttggtattgcttataataacttattctcgcagcgtactcgagaacgtttgaattcgtgagagcccttaaatcaacgacccccggcgtttagaaacggcaatccatatacctgtcataaattatcttagaattattattataccctagccttagccattttgtttaccagaacacggatggatctagttacgattcatataaagtgagagaggctagtgttgtaagggagtgagagagcttgcatcttacgagctcttagctcctcttatcaaaatatcatttgggcccaacaacgcgtaagtcagatgatctattagcagtttggatatgttcaagaagtcctccagcgggtttgcgagattctctgtatcgttgacttgtgacatatgatttgtattccaagacggtcagttgcaatcttgcctgaactagttggattatcagccaccccaggctgttgcatctaattaagttttcctatctgtaaaacctttcacttagcaatggcttaatgctcttaccgatcagctggaagccggtagtactgtcacttggttttcttaacctatcaaaacggaaacaagccgtatttttgatggtagcacttcaaatggtgggcaaccgactaaagaacgtcactctttaaattctcataagttaaaatcggatgtcgagtcaatattttgtcgggccatgggaaagagagcagtatgctaccttcttaatctctaccttactttagacaagcatacgtcaacaactgtgactcttcaaggacgggtattccctgactcaatgctttggaagaacatttaactgggttccattatagtggtcggactctttatgcttatgtcgcaccaggtccatctatcgaattcctgtattctataaacaccggctgcactctaagaaagatcgagcttctgattccaaaagtctataaatgatcagttagcctagcgccgacacattgctccgttagaagcttgacgtttgttattatgagggatcacagattaccgtgtgtcgattggtggctcacttatctatgagccagtttcgttatggtcatacctttaattaagggaacatcgtgctaaaatttttagaatggggtactgtctagactgtctcgaggattcatgccgatgaagacctgaaatttgaatcggaacttttgtggcaccgccgtatcgcaaaatgagaaaaagatatcgttaaccccttataaaccgcaactaactaagtcaaaataagtcgacgtgacttaagatactgattaagaaatggtatcacggctcttttgcaataccattaccaaaattgccaatgaaactgttttggcctatcttaagccacgaataatat

SEQ ID NO: 35 - DNA, 2000 bp, synthetic construct
  misc_feature 1..2000: note = Description of Artificial Sequence: Synthetic polynucleotide
  source 1..2000: mol_type = other DNA
attcttaaagtcgattcggtgtcataatagggttatctaacatatgtacaaacgccctataaagttattatcggactggtgcataagtaacagttcgctataaagttaaatgctatcaagagaaataaggcatactgtgatgaaaacgaggtcgtacagaaacacctgcaggaattaatctgccgtatcatacaaggaatatcgttggagtcaagatgactgcccatttgcagttgtcatcttaactgatgatggtttcttgcttgatagcacccgcctcagtaaaaacagatggaacactccaatgctagccaactgaaatttaacgttagtaccaaaggcatccaagcagtcccctggctaagttggagtgtggcatcgatataaaatagttaaaaaaacggtctgatgtttcatgcagtcgcaaccacgcatacggttccggttcgcaacgattgatgtggcggtctcagtattttacaagttttaacatgtcggcagccgctaggtagatacctgcaccctgtggtttcgtatatagggaatttcggtgctttaagataaggattactcataggggatattactcgattgcctcgaaaaatgcgatgagtctctatattcaacggtctattacaggctttctattttctcgggacgcctaggagttgaatgatgcacatcattaagctacttatgcggtcttccataccattccaatgtcgtcgaaagaggatgcagtgacaactcaggatactaataattccttgagaactgtctatttcaagcctattctaacataattagttgctagccatataagaaaatatcatcaaacagatagggttgataacagagggtgctgcccgtatagtgaacatcgtaaccgggtttcacatcctagattggtggcctcctactatgtaagatgtagttatactgaatgtggtgttgtgatcaagacgtaggaaaatttatcagatatgccaactagtatcatcctgagttataaagggggtaatttcggacaaaggtgttgtttcaaaaggttcaagccgacgtacccgcacatcaacttatcttgtaatgattcaaggtttatgtagcttgatcaccaagcaacccaagcgagctgtaccagatacgattatgttaataaaggtttggcgtactagacttaacgctaaggtttcgtaatgtaacgcctgcattcacgtcaataatagctcagtatgtgagaagtccgatgctgttaattctaataacgctcccacttgaaggagaaagcgggagtaggtgcgtttgttcagaaaccacttaagcggtttgtttgtacgtacaaaatttgcttttagatgtatagttgtatacataaccatcgtccgaaagtaaccttcatatgaaactcaaaggcattagttgggaagcagtatgtggcgtttgtgacacatcgggattataaaattccaatatatattctaagtagcagttaaatgaactccactatggttaaatacttgtacctatcgttattcgcaattgtgccacttttacatagattgtgaaccggtatatcgcgtggtcaagaccaggcttcaaagctgtagagaactgtttattctttgagtgacatagtatcgagacttgtataaacatggatggtacacaacgttggaaaagccgaaagccaataagatatttaagcattatgcttttatgtcaacactgactttctaaaccacacaccttaaatcagtagaacagcattttgaaggagtggctaaaccatgttgcgtgcaattctccgggctcgtaaaaacgtgtcgtgctaaaggctctaaatctcgcagtaaaggaggccctccaaactaacttaactcattttgacgaactcaagtagcttctattaaattcgtccgaataccatgaagaacgggattcgcatactgcgttcgccgtagtggagctcgttacaaatcaaatggatcgataaacaaacg

SEQ ID NO: 36 - DNA, 2000 bp, synthetic construct
  misc_feature 1..2000: note = Description of Artificial Sequence: Synthetic polynucleotide
  source 1..2000: mol_type = other DNA
ttagtatagttaagataatgcgtcgctaaacaacataaagattctttaccgatgagttctcgctggtattcgcttttttagtcttactcgctcaagttatcttgagagatgtggaactgaaccacttgaggtagccccatcaattataaggaaattgaaataggatcgaaatattctgaactatttccatctagtctactgaaattaacattgacacctttcacaaacgaatggcaaaaaaggacggatccatccccacagacaacttcgtttatttcagcacatttgtccctggacaacagccgtatgtggttcgacatactacctgatagtgagcggttatcgaaatgtccttgactagctactaagaggctttatacaatattcctacacacatagacccagtagatatgagttctagttggagatttttcaacacaattacgccacgaggtccgacaacgtatcctccacagttaggaacatttattacaaggaggttagctccgtgctacagcaacacgaattactccaccgtgttgagcaggtaaacgagggcaaaatacaccccaaagcgtaactgcatacgactttccgctcgaagattgttaaaacaagactgcaatttctgtggcaaaagacactaaagatgacagtacagcacccatggagagtttgtacccggttcgacctaagtatctgttgtccagaatcgtgaaatttgaagtggcctaaaagctgagacgagtatagtagggtggaggtttcctatatgttggtcggtcagtaaatatttaaaccacgggagttaaacttatcttaaatgtatctatacattagtatataggctgagattcgatatatatagacgccaccccgagaaatagaaagatagtgattcaaattcctaacagttcggagtggtatacgcatttctgagtaatttggcgtacaaagtttgagtagagcacagagttgataactagagcaatgtctgagagtggattaacttggtgtgctctgctagaaatccccagtgatgatctctcataaaaagtgactgcaagactaggatacaatttattatcgaagtatcaagatcgtgggttccttttttcctggtcaaagatgaatctgtcttacttaacgaaacacaggaacttttcttgcataggcaccgatcttgctatgtattgaagctacttcaaaggacctatcagcgggtgtacacaatgtcggaacatgcataaatggcagaaggcgatgagtcatttcgcacaccaacaggccgacgagcgtaggagcgactcagaacactaccaactatagcataacgataaacggagaacgtccatgccgttatgtgaccattcggttcggagtcgtgggttaccgaccacgatagaacatggcacactgctttctcacttccccaataagaaacaccctggacgtatacctcgattggatctggagacagtactcggatccacacctaagtagtacctcactgtgggcgatggccaagacgcgaggttgactatctgcgtggtggaaaaggccgacagatctttatcaattgtagtgagctgatgagtcctttatccgttataagctacttttattgggtaatagatggtgctcttactccttcgagttaatatatagaaatcaccgcaaagttaaacgcaacatgagtggtttggattaacaacttctggaatcattataaccttaggagcgttctagtgatgctgaaattgagacagtaaaaagtgcccatgatgtaggaaagtcactataaagtgaatctcttgtccttaaacataaagcgcggtaaacactcacgttaagatggttgtggccacaacatgactcttgtggttcttgacgtgttaacgcggtggcactagcagggatgatacaagttgatgcttacccatatgattattgttccccggagccaccactaagccactaaatgaagatttttgcggcga

SEQ ID NO: 37 - DNA, 2000 bp, synthetic construct
  misc_feature 1..2000: note = Description of Artificial Sequence: Synthetic polynucleotide
  source 1..2000: mol_type = other DNA
gatgttctgaagttccttagcgtacaaacacaaaacgtgcattggaaaatggagagggaaccctctatgtctgatgattttttcggttgagctaattccagtgcaatcgacaataagggcatgtccgaaattcgctttttaatggtagtaggtccggcatcattatgttgtcggcctaaataccataatcattgctcaaccttcaactctttgctggaacaattagtacttttcgtttgcgcttaaccatgcgtataatgtaataaaagcaccagtttatagatatcggaaaatttagagttcatgccatagtttgaaccgacggtaggtacctataacgtcttttgatttccgcaacctatgtattgtaagcagttgtcctaaggagtattttcactgtctaagtggtaaccagcggcgagaacatagtcggcggaacggttctgatttcgactagcatcggcgacattgccttgtcaatctccataatgatataaacatggtcttttaactctcacaacctaaattattaacaggtcgatacttctctggcgaggttgttttaaaacttccactccggataggaatttcattgaaaatataaaaggttgatgtgtcaatcgaagtctaaaaagaatgaagattagtgtcgcctaggacatctatttgttttaaagtgcaaggaacgtgttcacgtagaattgtgaaattggatacatgtttagtgtcatgcattgtttatgggattgactataacttagatagagaactagttacccttattactttgcagtatatgaacgactgattgtcaagactgagcctaaattaaagtaatcagcacattttggatatggataggagctcagtttctggtttcactctcatcgacttctttgtccaaatacggcaatcacgtaatgcataaaaattcaaacataatgtgatgaaagaacatatcacccgtctaaaaaattaaatatatactatagtgctgcaatacatccttaaattgtcctatattggtaagtcaaacgatacaacctgcattcttgggggataactgatgtttactggacggcggaaatactttaatttataggctactccagtgcatagtaagaatcataatttggtagcgcctagtaaaaagaaatcctcaaaaactaaacgctattctgatcgctatcatcaagaaatgaattgtaagtgagggctgtattctaactcatcctagcaggatttattgcctgcatcatcgacattctgttcgaagcggtgatccccatttggacaaattcaaggtttggattatctagcgcccttggagtctctttacgtgtttaggtgttcctgtaggaaaatcatcttattgtcgcgaatagaaggtacaaaaagacctcaaagttaccatatgcaccatggagatgaaacggtaaaagtaactgggaccaaagctgtccttccgggattcattattaccataatcattaggcatcaataatattctgtgcgatatgttgctcggcttattaacctcaatgaaacaatatgaccgcatatcgctacagtaaatctacgacgtttttactgattgattgaatcgcactttttaataattgtatgccccgatacataaaatgtcataatcgagaagcatatagtagtattgtagtatcctcaggatcggttggtagctttaatacgtgtaaatttttctcgtaattatcgagagtgtggagacgtccgtgtactggattcgtaagaattcaataccctgatgtccgtccgagtagatcgataaagtaagtagggatattcagatatttaatgtatttcctgtacactgtgacatctctgcaacgagattgttatactggcggcgcgtaggaaaaattcaaccagtctgtttgcagggatagttaaaattcattagagaccagagcaaataatgagcatccgaaatgtatccaaagcgatatacgcgcttacaaactctg

SEQ ID NO: 38 - DNA, 2000 bp, synthetic construct
  misc_feature 1..2000: note = Description of Artificial Sequence: Synthetic polynucleotide
  source 1..2000: mol_type = other DNA
ttgatgtgcgaatataacattgatcatcagaggcaaggtgataggtattaaaacgttagcgtccacgctcctggttctataaaacttctttagatgctgctaagtccattgatttactgttttatagatacgagagtaaatatagtttaaattttttaagtttgaaatacgtgtagctatcgttgcgctaaggagagttgtctatgtactagtgatttcagtcggaaatagcagaaacatgaacctatcacatgactgtcgaatggaaaatttggagtctggaacattcagtatgagatatacattaatccatgactcagaggaattgacccactaatgttattcttagttgcaattccaggtatgtctagaatttgcaatcggttagccgttgtgtacttcgtatcaattttcaaacagaatacaaaaccacgctagttagccgaaattactcctaattgtcgtcactatgtaagagatttagaaaaaatagtatttggtactactaagataatcgctgtccactataaacttgtaggtagttagtcgagtgttctgcaagggtacattcatggaattcgcgagcaacgttcgcttctccccaaatattgatataaagacgatccattctatgtattttcgcactagtaaaatacctatctactcgacttacgctatagctcagggatctatttgtaggcatccacagctcagacgaaataatagatttacgaactgatagcggccctccatgcctgctaatcatgttcatacatccaaacaaatcgttttgttggtagacaacaacatagcgataatttcaactggttgaaatggttgtatagctgaatataaacgatcccaaaaaattcaagatggtggctgcaccggaacgacgttaatagcgtgaggaggtgttaaaagcaacaaaatcacacccgccgtcttctagggtaagcgggtgccagccgggtctactggataagtagatatttagcaaagaacctcagttatccattttctggttacgtgcacaattagttttgcatctgccggcttttgtctctggcacttgacaaacctagcaaaactcaactgaggggttaacacgctctaagattcctcttactagatgaggtattcatctgcgtatctgattctacgttataggctttttctctcgaatactaatgtctggactgatcaataagaattggctaattgcggaagtcaaaatagaaccaattatattcatacttctattattagttctaggatgattttcccgaccatcggtagtaggaggaggtgatgtaactcagtagtattatgctgagtgattgcacctctgattctattaatatggggggatgctgcttgcctcgtgggttagtgtccggatgaaaacccccctaacctattcacgtatagtatcccagtcaattgagtcagtgaccttaatcctaacaaaaaatacagaatgctgtgaatgacctcgttcttcttattgtgcacgatctgattcgaaaatgaacggtatagagtctgagcatcacgatataagagattcattctgtattatttacgaaaggcgtagcaccattcgatcagcgagcagaaccacggggcagtattgaatttccgtttttccgatttcaaaacggctagaaatggctgctggatgatagatgcccaactcacacggttgaacttgcttatcaattgtgcggttcatatcagacatagcagtctgcttggaagatattgagtaacttcagcattcaaacgcgcaaagctattgagttgcccctgatgctgtctatcgtgtattaagtgatcgtgggaattagacatacaactttacctcttctagcttgtttatagagcctcaccgaggtataaatcattaattacccaggagaccggttttgctattaccttgtaatgttcaaaaaagaagtggaacacagtgaaagcctcatttctcaagcaagtgagta

SEQ ID NO: 39 - DNA, 2000 bp, synthetic construct
  misc_feature 1..2000: note = Description of Artificial Sequence: Synthetic polynucleotide
  source 1..2000: mol_type = other DNA
tgtagacatttgtcttcaatctaacctctttctcacgaaataagggcttgtattgttccttcgtttgtttaccgcacagaaacagcttcacttaacatacattgtaagtgtgtatttctcggggtacgtaacataacgaaacttaaagcaatcagacatacagtgccattccctacggtactgtctcagtatgttaatactactcatttgcaaaaggatgtacgcacttcatactacagctgctgacggtgtatatcaaacaattatattaacgctcgtaggatagttcacgtccgccatatctttgatttaggcttcaaaattcagaataatacgaaatagtctgtctactaggccaaagtcacttaagggctaagagtgtaatgagtaatcaaaataataatcgttgagtcgtcaattggagcatcagttatggcattaaaacatctagtgggtcgaaaggatcaggaaattatgtatgggtgagagtcgctgctacggtatcgcttttggattgagggctactacactcagtacccacagtgtgtgtattaataagaatcgcaatatgcgtccttttaagttttaaggtaccctacctttcatatctagtggaaatcatttacgcctatgcgacaaattagagacttttatttgtaaaacattggatgttggaatgaccctagatgcatgttaaatagcacgttcattagtggtacacgcctatcactaacgctatggaaaaatagaagaagccagaacaagtaaacctatggtgacaaataattacataaggaaatccctcataattagaataccataaaacgttagttgtactatccgtaatctaccttctagcgtggaatagttgagtgtattctagtcacgccccgttccataacgatacatgtaaaatttacagcgacgtttaggaaccctacaaggggagcagcagcgaggatagctgactagccttacaataagcacccatacttatgattgacatgatggtcatgcggcgttaccactccgctagcgttacttctttcgtcttgtaccggtttggcaatgcgatgcagcccaggtaccgtagagaaagtagcgatgtgtgaggtcgagtactttgtcagaaagcaagtcggattgcggtcccatttaccgcgacgtgcatttgtacagtatgaccgttttttaccacttactgatgaggccagactaataaacgatatttggtcacaggacaatattacggccaattatgaaataactgactggcctattgaatgactaggaatgtcaagtccagactctagctatttgggaggtttatatgtttggaccgacttgtgggagtttgacactacgagtaacaagattatccctttttatgctgcgctagttgacatggattgacgaggttattaatatccatgactaactcatcacagcttcccgagccgagacggattattttaatctcgttgatcgatatattaggtgacgtgagaagaagatgtgtcgtaatcagtaatagttaggatcaagaggttaaaagaagcgccttcttcacagattctcagtatctaccagcacagagttctcagtttctaacgtgttccgtatggatttgcgccactttctgaataagtcttatgagatatacttacctggtccagatgtagcagcgagttaagattataactgcggtttagcacgcagcgtttaaatacaaatactcttgactgttataacgttcaggattaggaacaggttcctcacggatatagaacccaattcacgtgcatgaggtattctatcttagggggaggaactgcgctggagcttgaaactgaccctctaggcgcttgctttcactgagatctattcaaactgacgtttagtaagaaatcataagacttatctacgccgccttataatttatgttattaaaacatgatcatgcgatcaattaggtaaatttctttgtgccttgcaatatg

SEQ ID NO: 40 - DNA, 2000 bp, synthetic construct
  misc_feature 1..2000: note = Description of Artificial Sequence: Synthetic polynucleotide
  source 1..2000: mol_type = other DNA
cgaatatttatttttcctacgcacctacactatcgtgaagttcatggtatcaattatatgtcactagagccacaaatacgtacttaaatcatttacctcgactgaaggttgtaggcttggacatactcttgccaccattgtaacaaaggtagatcggttggacccgaaatttggtacttttaatctagaatcagcaatatcctacggaaaggcccaagagatgtctcaatggatgagagtgtaattacctaatttcagaaaagagagtttaacacaaataagaacagacgaatatcaataaagtgcacgtcgggcctaaatgagcccacagcctggatagattaagtgcgatacgtcgctaccaacgaacaaaagtatttggtattatgacatcggctccgacggtataggataggaataactcccaaacaatataatcttggatacgattaagtttgagtttgattgatcccatcaaacatttgttggtataaagttaatgtgtgatccagttagaattatatgaacatagtgttgtcacgattttgagacgaccgttaaacattatactgcggtggcatagcaagttcatctcctgacattagtcagcatttaatagtaagcaggagtactattaacacgctcctataatcggttgcctgttggggataatcagaacatgaaaaactccatattagaaaattacataatatagatcacgtgtatgaaacctaataccgcgaatataattacattatgattgcaatacatagggtagactcctagttaacgtaaaccaaataaccgactcgagaaacacaggactaacaattataatttataaactaagagtgctatactagttactgcctgatacctatgtttatttgcaagtcaaaagtttcaaatagcccttggcaagctacatgatgggtgattggaggtgggactaggagttccgtccttagtctgaataaagaacatgatgtgcaccgatttgtcgtctactcggacgttgtggcaagaataaaagtgaggtatagtaccgctagccgcagagatactgccttcatatgcgccgatactctattgttcataaacagcaatgaggcagagcacataatcttaattattaatttagttaacggcttcccaatttagcaatgaataaattttttgaggtgcatctgtgattaattcacccagaaacgctttcgcgaattacctgtcactatagatccttaatgaattatcttcgtcgtcggaacaattatcggactttattttgcctgttttatgtatcgagttaaataacgggaatcataattttatattacatctgttttgtatagcggatctcagtaggttacatcactgtcgtcggattcaacagcaacaacaccgttaatgaatatagctacactgcatgagtcccaacagcactggtccactagaaatatataattatacgaatactttgctatgttcatgacctgtcaaaggagaaatctagtaaagacccacggatatcgaagaacattgtagttctgactcggtttgaatgtccggtaactgcaggttcccgttatactgagcggtccgaaaatggcagtctaagtccccctacatgacgattgctatttattaggtctcagaatataacattagacacaagagcacaatagtcggagtatgcgttatcgagaccgtatatgagtcaatcgaacgtagatcgatcatagctaactaggtggtgtatcactgacgacttgacgatgttttatcgctgattagtttatgatcttgtaaagattggatgctacatattatggtaattttgctacttcccccaactataccaaatgactcactgtttatcaaaggtgactggataggcgctaggtatatcccggtgcgcaattattgccctggcgagccgaacatctcgaatatgtaaagacgaatactccctaattaccttttcgaggtaacaatgaata

SEQ ID NO: 41 - DNA, 2000 bp, synthetic construct
  misc_feature 1..2000: note = Description of Artificial Sequence: Synthetic polynucleotide
  source 1..2000: mol_type = other DNA
atcgagttggtttctacgagtagctggcaagcgcacatagaacacacattgcatgtgagtggagcgattgcgagacgaaacaaccttccaaaagcccaacgattacagtgctagttatctatgggaacttattcccttagggccaaagtccctaggttattctatacgactcacaccgaagaggctgtaaattaacccgaatatagatgattagtcctttgtttgtcttagggatggcaccataataaaattgtcaaattagggtacaggactagttcgatttcttctatccgtcgtcctaggtttatatgtggccgtcaccactgtatcacatgccagctagcaacagtatgatgtatagcggcaaatcattcgtcgggggcatgcagaacgtcagttaactttaaagatgagactacgttttggtcacaatacaatgacttagactcatctcttaactcagacaatcacttttatacttagtgcaatgtgtcacagccacttaatggcctagctaatccttatagtcggtagctagcgagttatagaatcttgttgtggataatcctgctcaaccttgcctggaagtctaagaccagtactagaagttaggcgtcggagtctgtgatgctaaagttgttcggccaactaattaggggtgtacctccttgtctaatcctcttagaaattattcgagaagggtacagtacccctcacaaagagaatctaagttaccgtctgaagtctgagtgatccgttttgaggtaaacagctgttatacatacttacagcttagtctacatgacctactaagcgcttcgtgctccttaccgtcccagaatacccatggctcgcgtctcctgccgtacaatacgtagatttaatactcgtaatgtttacaaaaaatggctcagcgaatatgaatacgatatacagtaccatatttatggatacaaaatttgtggcatccgcctaatagggctttcctcagggcttactccacatactgttcaaccttctaggttcagtaaaagtggagaccacgatgcagtgtccttcttaatctggccttatttgtcgatcccttatctcgctaagattagtcacacgacaaagaggtcgttaatgacgtatctagccacaatcgacagtcttctggcgaagatatctacaagagtcgttgattcgtcacttttagccttgtaaaattgccctttgaataggtgacacccgaatggattggtactttcgtaattaaccgagactttggagaattgtctccggcgtttcatgtggcgaagaatagaggtgactttgatggcaccagaatctcactgacaattgctatagacctaatatcggatatttctgcaacttcctaatcgaaaaaatttctacaaaccagtcgcagccttgagtattcgcccttgacatagattcacaagattgagtcgcaaatggtcctatgataatggatgtgttattgctggaactttatcatgatgcaaagaggttataatattttgtgttagtagcacacttaatgcacgcagaatccttaatcaatcattagctgctaatgagaatcaaccgaccgtgttggtgttactggaattatattcagtatcgctctgatcttaaggccctcagcacctgaggtctaacgaaaatttttttaagcccattctcgcaaggccacaaccatcagtctctcgagaacgacattggacctcatatccaagcctccggttattcaccgatgtatttcttcgagtatctaaaatctgccaatacgattcaagagaagttagtatgcgggatcatgtagcgtacctttatatgaataaaacatacctggtagatggaaacttggtgacccgggagtacgtcattctggtactgatacttgagggtgaacatggtgcgtgattccagtatagcggtgaacctacgacaatatgtgcatggcattgcttatttggtgtatcgttttttgagaa

SEQ ID NO: 42 - DNA, 2000 bp, synthetic construct
  misc_feature 1..2000: note = Description of Artificial Sequence: Synthetic polynucleotide
  source 1..2000: mol_type = other DNA
taactatatggtgtctgtttactacgattgcattaagatttctagcaatcttctccagtaactgcacttccccatattgtagaagcgacttatggagctaatctttcacttggtttaatgctaactgggatttgagcacgtaaaacttaactcggaccactttgttgacataattccgctgcttatatacccatattcatgtctacgattataaagttcttcgtatttggctaagcgtctctacctaggctcaagcctttttagccaatctgaacgctaaacgggtgctagcctagtgattatttaatgacgatttgagttcatggacgaaattacattattactgtctaaccggacaacgggcacgtcacaataagaagggtacagttgggatcgcagtttattcatgctgtatgccaattctactacctctcgtcatcttaattcatatatagctgaagggctagcaagtagtggatgactataatcgggatttagaagagttttttcctcgaacattagccttatgtgtctattttgttaaaattgacatgctaaacgatagctattagctggaggaataacataatgttgtaaaaggtaaccagctcatcacttcaggaatcttacttcctacgatggctgtcttttagtcgacgtaaagaaacccaaccaaggaatacttagacagacaggagatcatcctacaaagatagtcgatcttttatttagtccaacgcttaccaatgaatagggctgtctgagactcaaaatattggaccatgggtttcgcaaagcgcaaacggagaactatgatttcttgttgtggcagcgtatggtccccacgggtgactgtacaatcacggagacttttatcatataacgatagtacatttatctggataccggatccttcatttctcggaactctatacttactttaatttaatggcccgaaatctattatccttaaattacaccgccgtggactcggaatgaagatgagtccgcaaggcatactgttagatcggctgagatattgcctagtgcaatcgatcttttgatggtatttgtgtacattctaattcgaggcgaaactgtcaataaactaatgggaaaagcaagcatatcacgagaaatattctaggggataacattacgttttcggaacacaacaggttcgacataaatcttttatcatattatttgcttacaattatttagggcttccgcccatactcagtagttcaaatgatgcaaaggatgtggtgtctagtagatctcttaaatttctatcgaatggcgtagttacattgcagttatttttacatggcaaaatgatcaaatttgtacgcaatagcagtaacatattctctgtagtctatatctttatgattggagactgttaaaagctgatatgactaatcaagaaaatatcgaaatttgatctacgacttaacattttaactaagcagacatcataacgtttattcttcaacgggccgttactgctaaacattaatctaacgtaaatcggaactctgcagagtgcccgtctcttattttgtctgaattttagaatttacaaggagatgctcaagccgagttagaagaagagaaatataatgaatccaccgagtgtatgtttatacataaagaactatctttaggcgacgtgctagatcccactatgttcatgtgtaacgcatttattggtggaactctcgcaaaatcttacattatttcgccattacgtctatacaaaagctagatccgtgaagggtcataacctcctttaaaggcatgaaagaggttatctaacttatgattctataacatcgtcactggtggagtaaaaacatctgtgataaatacttgtgatactctctaacatccctgtaatatgatgatcataacgcttgcacctattaacttaaaagaaagttgtcttatggtgattcttaaataaaagtgcctgagccaccttgtgtaatttttaa

SEQ ID NO: 43 - DNA, 2000 bp, synthetic construct
  misc_feature 1..2000: note = Description of Artificial Sequence: Synthetic polynucleotide
  source 1..2000: mol_type = other DNA
cacaatagtatagggacgtctattattgaaaattataccatgtggacatattctggatttgaatttattttttacgaacttactcgtctctttgtcgaactgatcgaaccatgataggcggtccatacgtgtagtgtgtgctagaagcatctgtacttgtattgaaaggaacaaagtcaaccatgctgttcaccaatttgatacgaaggaatgtcctatctaaccgggcttattttacaggctaagtaggtgaataatgacaggaaaaattcgaataaatcagaagagttttaagtaaggctcactggtcgaacggtgataatactggcggcaagttctatgtagcttattagataactcttcgggtgagagaaagagcttataaatgtggcgctgaaatccgatgccagctgtagccgagtcgcgtcatctcctaacggatcagttaacattatgcttactggacgtaaagtggcttgtctagctctcatgcgccttgtaaagctttttctcactgtgttcgattatagtgctctcagcctaccgttgcaaacaatgactagcgactgagatgacaacacgccacacatatcgagtggtaccgtattgggagggtagtggagagaccacccgatatggataacacgtacaagatgtggttaaagagccaatcacaaattgagcggcgatcgtgtcgacaatttttcattgtgtaagcatgcatgtatactagaaatagagtaatacttagcatatacgattaactcttggtgagatgagattctagctttaaaagaggggataccgatagagtaatacatgttcttttgagcaaatgggttgttcgccctgatccatgataacgactatttcatagctctaatttagatgcttgacccagtgtaaagatccgttttaactaacttagatgataatgagaaataaagtaattgactacttagtacactttaaatcctccagtcgatgtgtattgtcgctatatcgcaacccgatgttcacatacagggtcctgactttgggtataccttagtacgtaacaatctcactcacaatcaatccaagcgcggttactatgttacgacggggaagcaatacacagctaggcgtgcagtactgctcttagctctccgaaatctgatctagatgcccaaataattttgtttccaaagctagcgaggttttacgaccagtcatgacagattctgcagttgaagcatgtcacaggtaagcaaaagcgtggaacggatggagcgagtaatcaatagaacttactttacgagcggtgttacaaaattgggtataatgcactagccgacatcgatggtgtagtgaattggactggcaccctcaaggcctcgcccaactcagtctcgctagtttgctacctgcatcctatgaagctgtttttaaaaatatcgatttctagcggtagttaaactattaggaagggctaaaacaaagttaattatacttatgtgaacttacaatttatatattagaaagtgagtaagcatatctgaacaagcatcatcgtaatgaggtcggttcgaagtataaacttaagttaacgacatcttccaataccatcgaagtctactaagtaagttaggtgcttaatgatcattcatagtgtagcaagtccccgcaactagataaagtcaacgacttaggagtttagatagaattgtgtaccactagctcgctacaattggtttgtctagacttaatcccttacctgttgagaccgactctatttcggtaaaaatcggcaaaatacggtaacattgtctgcagtctgaacacagactagcttatatacatggatcaaccatcaggtgtgactatgttttattatatgaactgttaccatggcgcctacgacaatagtatatttccatttcggttaccagtttttgtctactttatccattaagtgatatatatacatgtgtccaacgttatatggacagcgttgtgca

SEQ ID NO: 44 - DNA, 2000 bp, synthetic construct
  misc_feature 1..2000: note = Description of Artificial Sequence: Synthetic polynucleotide
  source 1..2000: mol_type = other DNA
taaaagaacggacatggcgcacaaaatgactatgaggcggttacttctgatgatcacaccctagttcttactcaggctattgtacaccctgccctctcaatatacccggaaatatgcatttatacggcaatcgatcttgaatcccagttcgagtctttacaaattccatcgtttactacgcaacgtcatgctaaataacaccttcccatatatgtagcgtgggcgggactattagagtcactttgtgctaaacagccggtaagtataatagtttactccggaaggtgtcaatatgtttagcgactgtattttggtactttatccctaaacttagctaatttacacatatagcagctggaggagcaaggtatcatttaatcttgcttaagaccctagtttgtacccctgtcgcacactaaacccaaaattgcgacattgagccacttaggccacattcgttaatctggtagttacagcacaatggctataatatacagatacgtctagaaaaaagttatttaatgcatagcttgcataatcgattctttaaaacagggtggggagctacgtatctaggattttattctacgtcatgataacgaatcttcctgaacgtactagatggcgactatcggagaatgatttagaacgccgggtgtgtcttgatgatataacaataagtaccacgaaaagaatgtaaataacttgatatcgactgtcacaatttgtttgtatcattgttcgtatcattatgctcctgctcgtgtcgcaattcccctttcaccttttggttctttatacacaatcatattatagacttatacggaatattggttgtaacttagagtaataccgattgaacccacatgtcgctgactgcgacgctacggcatcttaagccgatatatcgtcgtgacgtaactaggagtccgtaagcgaagagtagcatagcgatgatcgtttcagactcggagtattagagttaccatgctagccacatagaacggccttccgtaaccggtggcactcgttcgcagtgggaagcccaagttagaataaattgctaaatctgattctcccgtctggacttcgatcttcgagctagagtgccactacgggcactaacacattcaacgagtttcgtcgggtggctcgactatcggcacgagtgttgctctacgagaatacctgccttccttactgcgatttctctttacgctcttccactggtgccaagtggctgtatattactggtcgagtagggctcgctgattgtcgtgattcaaaaacgcaactctaaaatccatacctttgttgaatacctttattctcgttatcatagaggtgttcgggccctcactatcgatggcagatatagcttctccgctcgtactttcatatagatgttccccaacagctttaaagttagaatgatccactttcagggcatccagtaactcgagcaattatgtatgtaaccgatctttcgatgataggggatagtacaccttaacccttgtccccggtgaattgcggcgacaccatgcggtaggcgtatgtacggtgtgcccttaattaacatcgctactgtactacacggttaggtcgtttgaaaaggcagccatgaatgttaagatcttattttaaaattgatcatttacatttagctgctttgggggtaaatctactgatccaggtattaatctcttttgtataatgtaccaattgtagtaggttctctatgttcttaagtttcattgtcgataataaactaatcggcaaaggaagaaaactcaataacttgtattgtaccaaaaaagcgggggctatagttagatcggtgactcactttcttcgatataagggaaacccaccgtataacgacggtgatcttaagccttctcccaggttaacgtatagcctacaaatgaatgcattcaaaatgtcgtaagccttttacctggaaagcacaaacgatagcgcatttccttaaagtacct

SEQ ID NO: 45 - DNA, 2000 bp, synthetic construct
  misc_feature 1..2000: note = Description of Artificial Sequence: Synthetic polynucleotide
  source 1..2000: mol_type = other DNA
acttgcacagaaatgacaaagacgtcgattcacgataaggcattccaataagtataacataatcgtgtttcggggcgcacaaaatagatacccaaaagagtgtcctttccactcgacagtagagctcatagttccgtgagattcttgcctcgtaactagtagactgtctatcgcaagaatatcacacccaatatttaacaacgctctgacgtagtagtggctacttgtgcgaataatctagtttctcatatttgcgattcaacttacggctaaacggcctcatagtttttccctattttgaacataagtcgctgttaagcagagtgatacttcccttatttaagtgtaagatgttaaacactaagctagaacacagtaagcccccgtatcttagacgtaatagccctgttagattaaaggattgcgatcgacataccaacagatgacattaaagcaagtatagcttcaattcccgccacggtaaacacctatcacgatacaaaggatagacttaccgagtaccgtagttagtaacctctaagctagtaaatcaaagttttcgctagttattcataagaacaaaattacaaaatgcgtatttacaactcatttacagtgatgagaccgattctaatccaatcggtgttagttttgcttatctgaaaatactgttagaaatgacgtggctgttaatcaatgtataacgtgcatgcgctgaatatcaatcatcagtatcgaggagttggcatacgcgggggctgttgttaaaaattgatccgaatcatctggtttactccactaatggattaagcctcctcaaggcagctgatgtgaaacccaaagatgtcaatttgatttcggtaattaattgaaatccctgtcctgagcagactataaacagataaccgtatggaaatctgattccttagacgttttcaaatctattcaagtaaatttttacgggaatcttaaacgatatcgttccgtgaagtaattcaaaaaacggtcttgatcttataattcacgtttgatactaatttagtcctccgctccctaatgattttttacgaaatggtccagtttattgtttttaaaactctttggaaaattcgtgtatgaggatgataaattgttcgatcaacgtttgtatacttagatctcaagcaagaactgtcagcgacctgtcgttaggtagtttgttgcctgccacctcgcgaccttaggaaaggaaggtaatctattccttaatacgtactatgtacaagagatgcaagaaaagggcaacatgagaacggttagtctctttgaccctcttactggttagtgaatatttttaccagctgctacgatgcaggatatctggccctttgactgttccatggacacgagcccgaaggatatttatttaatcgagagctgtatttagtatcttcataggacttgaaatcggataccgctgtaattgtggaacctcatgagacctcctaacaaaacaagtatcgacctgccctatctccgacatttactcaactctacccccaggttgacaatttaggatggtgtctatgggaaatatgattcgtaacgtgctgcctcaagaataggttatgaaaatatatatataaaattctatgatagttccttcgtctcactcaatactaagtcgttaagccaactagctcgggcgggctattagttgccatatgaggatccatgaatcaaacaaataatgcaattctgctaaaaagtgtgtatatagagcgtacacacaagaaacaaaactgaccgatccgacttaaccatttcaatataatgctgcacccttgtcctcaatagcttgcagggggcaattacgtttggagtctggttgtggtaatactcgactgtcctcggcgatatagaataattatagagtgtattatagcacaaattattaatagattccatagcctggcgttacatgaatattctcagttaaagcatttgaacgatcaagtggt

SEQ ID NO: 46 - DNA, 2000 bp, synthetic construct
  misc_feature 1..2000: note = Description of Artificial Sequence: Synthetic polynucleotide
  source 1..2000: mol_type = other DNA
aggaacaatgttaatatcaagtcgggtccaaaaagatgtgtaaagtttgcgaaccgttgcgatctgtttctgtatcgtcttacactgtcagggcactaggactcactacgactcatatgtacattgtttagctcactccgagacgcttagtgaatcgttaataggttgatttgttattgaagctgtctgacttattatcttcttaaacgactttttacgtattgggagtcataggcgttttacagatatccgcgtcagtccacgacgtggtgctctatcggataggtacaatcaacaagaatgattattgctcatcttaatttactatgtgcgccgtttcaccccaaattcgctcaagctcagaccattgagggcggaataggattgaggggtagtgaggcgctgctgtattaggcaaccccggtggttcatttgaaaaaacaatcgcggaaacaactctaggcctaaggggaacaatcgctttgactatgagcttctatacctttgaatatacactttgcgtggagcttggcgcgactccttttgaggtaatgcgatcctacccattttgggttccctcttaattatattatcggcttttgtcaccatgatctcataatactgataagttacccctgatgttacgaccccgcagccgttagatattttatttaggaggacctacccaaggcctatgatcctttctctatatcacgaggattacagacaagagatgtgtaatccgcccaagttactctactcaaggttgcgcatattaggggagggcgtttgacagttgcagtatgccatcttggaaggcaacaataaacggtacacaactttacaaatattccataattgtttctacttttcattcattcattatgtatccctctatacttataaaacatgtacgacatgtcctgtagagcgggacctgttcccgctcatgacagacgagttatttgtctccgacgtatcatccatctttaaatattgaatagcagcagcatcaagtgtggataagtgcaagcactattaaatccgcgtgaactttcatatgacatgagaatcggactgtctgttatcgtaaataaacccgagataatgttaaaactattctaatgacttcatgaagcaggatcatctaaagttatcacaagaggtggtcttgagtcttgcaaacttcagaaaacatttacaaacgattcaaattagcctaaaccacttacttaaccactcatattccacaagttacggttctttagaatattaaggtgtaatgacccatcgagccttatagctcgaatcaagattaaaagaatattctaaatgaccataccggttacatgtgtgggcggagtcaaaagtttttctgactattaggtgcacaaaggtgttcagaacttaaccaaactcttagcacatttgattagctagtcagattaaggtctccactttcttttctgtggtagttcggtaaattgatgggcattaacaaacttaaggttgattacaatggggggttatcggatggttattgtaattgacccgtccatagatttgcttaaaaatcgcattttgaatacatatcctaacttccaagcattacacagcgctgcactatagagctaggatgactgtacaacctcggattatagcttctacgtaaggcgtggccgtggctggtataatagtggggtggagggagaattgacaaaaaaagtttatcatttaaatattagtaatggggttgtcgttctaggaccgtatttcgcgtactaagtcacatacccttatatattttccacagcaagtctatcattgcaagctgttaacttcattccggcggctgctgaaccagtatcagttggtccacagaagctaaagttagcaaagtaatacacgccaacctacttatatatgtatatcgtatagcttaattgagatgtcgtagccattacatgctgagccttatttttgaccgagaccaggtacac

SEQ ID NO: 47 - DNA, 2000 bp, synthetic construct
  misc_feature 1..2000: note = Description of Artificial Sequence: Synthetic polynucleotide
  source 1..2000: mol_type = other DNA
ttggacgtcgaaattatttttgatatacgtgtaatgatagactaaaggcaaaaagaaggagtataagtctaagttcgaagaggcggatttggttatacgtcctgcacctcttgccagacattcttttaattcttgtgacctggacttgaagttcctttttgcgaccatttgtgggtttagtacgaaacccccataagcagttagcattaaaccatcaggtttgactcgccacattcgctatcgcaaatgctactaattcatcttaatctgacccccccgggaaggaagccatttaatagataatctgagtcgttccagagatgtacttctcagataaaccgtgaacactattacgacatatgctgaataaccagtatgtatggctgttgtcgactctcattcctatagtggagagaactgatacatacatattccctacacggatgttaaagagtcgcaggacctggtgaggcactggatcaacaagttgccaaactgagtgccagtggagctaatcacaccttcggctctgcgttacatgcgttagtgaaggtccttgaggtgtgccagcaaagattgttaacatataatctaagggattatatggtgtatatgggactgaaaacctagaggtctgtggggaaagaccgtacagtccctgaccatcacaataaaaaatagccaaaatagcgtgccattctaaaattttaatttttaatcaatcgcgactcctttggtttcatgctagttgattctatttaagaatccaagtgagttttaatcttaaccctaatgatttaaggttccagtaagcaaataaacgactcgccgtaaagcgaaattgatcgatacgtttcttgctttatttttgggtacagcaatccttcgaaatgttggcttcgtaattccctccagtaacttaaatcagttaatttgcattgtaagaaaacagcaagtgaatcatgtcgccgcttcagtaacttactgcaaaatgaaagcctaataaatagttacccatctatctaagtataaacgacttttgcttatgtccacccatgctaggctgtgaatcctcttacgtataacgtgctttgcgtgtactttcgaactttctaagtatcaatcgcaaatcgaagtaacttaccaccgctcgtaggaattgcatgttaaaaagggttaactcccttcgctttgtcgtttcccaacctgatgaaggaaggtgaaatacaacatatggaatgatatatatcacaaatacacacgactctggaccagtgcaaagtagttataaactcaaaacgcccccgacatacattaattctacttcgaaaaatatgttgccctaacgaaatggtttgcctaacagcggcaaaagatatgtcgactcgattgtatttaaatcgattattaagattgggatgagggccacgtagccgaaactgcaacataccgaaatgggcgttacaatgcattaattataatttattggcgctcagccttaattaacaatctaggcgtgctcatactgtgtactttaaagcaccatttacatgtcataacagattattgatgttacgtaaaattcatagtatacagtatcacctcgatcaaattcatatgtttttattttaaacaagagtactcctgtgtcgttctgaattactattagtcaggtgcgttaagctctgcagaacgataccgactatctgtgcatctacctgattcgaaaatgaaggcgattgggactctccactagttctgagttgtcctcctcgatttacaaaagataacttcagctggatgtttatcgaacgcacaaatcttaacaatggtttaagtagccgaatcagattcgccattcaaatctttgctctagtttcatcagtccgagttactctcaaaataacaacctaactcgtcttgcctacactggttctgggttttatatttagagacataatcacgaaacttcatgcactatagaaggcaccatgctgttc

SEQ ID NO: 48 - DNA, 2000 bp, synthetic construct
  misc_feature 1..2000: note = Description of Artificial Sequence: Synthetic polynucleotide
  source 1..2000: mol_type = other DNA
tgagcttcgctttttccagagtcgctgactaaagtgaagtgtctagtcgttgtccatgcgatatcggggtccatcaactagaattcatttacggtacgcgttgtcatgccttatatttagcaataagactaacggaagctcctctggagggaaagtaagaacgtccccccgggaacatacctaaaataaaggtgcatgaaccatcacggagtggagacgcaaaagatcaattagtacaaatcagcaggagacatgcaaagaccgcgcccctttctttttataccatcttaatagcctttactgatcgtgtatgttttcatcgtgcacctaattatggaaattctatgaagcttttgctcctaatcgtttagtaatgctctcggatgccacgttatcttactgagaagcccgtgaccaaagcatggtgacaatagaaccaatatatatgaaaataccgggttcgtctgaagactgtgtagtaacaaaggtattcttgtgaattcacgtttttaatctcatctactatcggatatgacaacaaactctgattagggtaatataaaatttaccgttcggcctaattaaaggacaaccggtatgtaaaacagcaacatcacctagcacgaaatttacctatgagtgtggaattcgttagcgctgtcgacgtgcataacctacgggttgttgcatacgggtcagtgggataatgttgactcggtccttagtaaagactagctcttcttattcttgcgcttgtaactgacaagtcgagttcacgtgggcgcagtaaagtcgggaagacggtaatcgcaaaagttcggtaaaactaacagtttttaacgagtccgtaagttcaagggcctaaatagctggaggattttaacgtctaaacattcgggacacagtgtatgacccgcataaaaggttcaaagaaataatacttagagccgtcgttcggatcttatatgtttgaatgaacccttaatcaccctataacatgaagctacgacacattaatcagatcaaaacctacttagagctcgtccgatactacaacttgaaatcttccaccaaaactaaagggtccattatgtcaaaataccatttctatttatattttaaccatcaattcgcctatacccctaatcagcattaatctcgcttaaagatggtagagttaaatacaacgcagagcttttatactaccagtgatggatcacaggattgcgtttcaaaaggtgatagcaattaccaatgacctttgacagtaatgttacatcctaaccggattatttggaataccctctatttgctttctgtttagccgacgcctgtaattgtctacctgcgtgcgttgtgatgccggtccgctcgatttaagcactccgatatctcatgtaggtgtggactttggacaaggggaaataactctcaatgacaatcgtactgcttatgttaggcaatgctggcatatgcaactctgaggctaactaagttagtcttgtccgtgatctcagaacagtaactatttagttgcttgcgagtatatttcggtagagacgtatcttctactaaacacggttaaatattttttggttatctctcgcccggtctagtagtgccataacgtttacgaggtcatataactgtcatacattgcaaggcgctttatctcaattgtgaacaagtaattatagccatgatacaatttttggacggaacttgttttatctaaatcgaaagaacctacattgcctcggcatagacctcggaagcagctagttcactagctgcttcatgatggtccaagcttgtgaaagattcacataaaatcaacctccgtgggagtctccgatggacgaagctgtgtgactggatattatctcatgattgcgtcacccttaacatgtgtgaggtagagctaactatagaaataccagtcgagttagcgacataatgcgaattgatccgcctgtcaattcctccttatacgcgccgtt

SEQ ID NO: 49 - DNA, 2000 bp, synthetic construct
  misc_feature 1..2000: note = Description of Artificial Sequence: Synthetic polynucleotide
  source 1..2000: mol_type = other DNA
attgtccattcttgtatttgaatcactccctaatgaaccaaactctctaagcccattcttgtagtatttaacacacatgacaacggtccaattttcatgtatagtcggagtaacgcgatatactgaatcttctgacttatcagacatataagatgtaaaaacagcggatcaaaagtgttctctgctgggtgaaaaatgacaattaagcgtggtattatctctgtaaataacacagggatttatatgtaaggatcgcgccctcatacattcattaattctcactcagacttccctccttcgggctacgttagattgaaatgaaaataacatgttgtaatcattaaatagtacatactgagtttttaaagtcgaatactacaaaaaatatcatactttttttaccagttcagtattggagtcgacacatgatctaacataacagaagacatagcgatggggattatcgacctttttatgggtagtaacaggtggttgccggatgcactagcatgatcaggtctcctactcacacagtccttctgactgttaggttgtctttgcttataaaaatactcggattattgcgccacaattatttgatcaacgagcttcttggagagaataaaaatattacacttcggatagataatacaggttaggttctcctatgaatttgaagatcccatgttcgttaccgtccaagagccacggcttgcttgctcgaaattaaagtgggcattcgcgcgggatgggaagtaccctcagtcttgacaattcccatcgtcaatattagaacggtggattcgccatcaccaggaaacgtattgctgatgatgatttcaatactgaagtcgtacacttctcacccggaaacgttaaaaggacgataatgacttaattgagatcatcgaggtacgagcccatgccttaggtcgcttcgtaggggtcctccttaaaggagactgtttcttacatgatttgttacttcgttgaaaataaatcatggatcgacgtcaccaattactggggtacctgagtatatagcgtagaacgtgaaagtgattacacctgtataggaaatgatgagctcggggaaccataatgaattatagtgtaaagataaaaaacttgccccgtgccacgagaaggaatgtagcagacaatcatggggacattgtaacttacccagactttaatttcgttttcactataccactcaattatgatgtgacattctggaattgatagcgtatgttgcagccttctaaactcaacactgagctccttaagggttattatggttatatttgagactataatataatccgagttcggtcgaagtgagtaatctttggagggtttaggggggcagaattcactataagcagcagagattttcttagaaagagccgggtcccgttccaataagccctaccggacgtttataatcattggtgcatcagtgaggccttctgttcatcttctattctgctgtacccttcttgcaccaacgcgttggatccttgtatcgagtcactgccaggtttgtggattttttgcagcccaccctacgttatatcttaacaatcggataattaaaccaagctatcgaatgctatgagctaccacagattatcatcgattgttttccctatcattacgatccctgacggactacttagtatgtccttttcttaatattcgttaagaactggagtacaggctgattacacaaccagtaggattaggattaaatagagaaatgtatccggaaaagcggagttactgtttgggtctttaaccgcgaatcgcggttttttttctaatatgcagtgatcctttatttggttactgtacatctgctgaacacgctatgtggatctcccacagttgcaagtgcaaaatattaataaattaatcacaatacagtacagctagatttcatactaaatgctgatttttgaccgcaccctcgagagtaattcaatgacggccatgta

SEQ ID NO: 50 - DNA, 2000 bp, synthetic construct
  misc_feature 1..2000: note = Description of Artificial Sequence: Synthetic polynucleotide
  source 1..2000: mol_type = other DNA
aatcagaatgagcagatgtaaaacatatttatgtaagcaggttatcccgtatggcactcgttgctctaagtagatgtttttgtctcgggtaacttatgtccccatcctcagagtgtatttacttttatttaacccgacggtgagaacatacaacgggtcaacaagacaatacgaccattatactgctaaactctcttcctcaggtgctatatgagttacgacacaatttttgatgttaaagtcgaccctagctgctaactgaacttctgggacttaaaactaccagaaaggatgaagaattagtttggtcaataactatatacgaaacgccctgaaggaagtcgtattaaatttggagtgcataagacatggtgagcgaaaactaacacctacctcttagatacagattacttttagttatcttctggtctatcgttgatcattctaagtttattcagcactagagacttttggaatacgactgccaaagctagtataggattatctaaagatcattattattaacggataatgcgaaatttgctagatcgtatatactattaatgcagcaacttaactaaagatatatttacagtggggcttatgcaaccggtgagccctcggttctttatgattcgtcaagtaaagttgcacaacgttcacgatttaatcttattctttgatcttgggctgatgtatcctcattatttatgatagaaaattgattggtgcatttgattcgcccgatactagacccacagctgttgttcgatcccgtatacaatgagagcatgttcagatcaacagtaggtgtaacatcttatgttccgagccttctagtaaccaacgaacacctggcaaatgaatttgccatctttccgctgtacgaataggggtaatgtgcccttgatttaaaatgttatcgataggggaactacagatactgagaactcctgaaacgacgttaacaaacctcctgcaaaacttgcactctttgaacgaggttgcctagtttccagaagtaggttcttgtcacttgaatttcgatggaattctccttatctatccagtgacgaggaagaagaaatgggtttttacaaggactaagtgtttagacagaaaaactaatctttcagtaaaggtgagaagtgattttgcagagggagattgtgttacgaggatagtactgacgtttatatgagaaatagttatcgataatgtgcgtgtctttaccaagggactgaccaactgatgtggaaatttaactcttcatgatcacataatttcaatacgttaacagttagaagcggtgatctttacaaagtagacaatgagttattgtcccatagcaatgcctaatgtcgagcgtgcttcaaacaattgaatggcgttattttttgatccttaggaaacaaaaaccagcaacgtaacttattcttgtatcttcatgtaatcacattaccggtatagagatggttttacatatacgcacgttactttgagatagcgaagcatacgaatatacacgatacaatgtcagaaggataaaatcactatggcctcactcggtgcatttgatttcaaaggcttaatgtagctctgttcgcactcgtggatatagttggagccagatagactaggaagatgtttgtttagatagtatcctcgttcgtgcataatatccttgagatagtataggtcgaatctccacagcagcaagattctccgtgagcattgccactctttcagtagtaagcctaagtaattcattaagcgtaattagagacttattttccatatctgcgcgtcgagtttcttctgcagccctagttaggagacatacgggacgcttgcgtttttatcgtagattcacttagtacagggaagataaacatgagaggaaatccgacacctaacaatactttcaaactgaggggctggattgtacttaccttcacatcatcgaagtcaattcttcaccttcacaagctctttcttcg

SEQ ID NO: 51 - DNA, 2000 bp, synthetic construct
  misc_feature 1..2000: note = Description of Artificial Sequence: Synthetic polynucleotide
  source 1..2000: mol_type = other DNA
atttacacccatgccgaacataaataaacaaacacaaaaggatgagaggaataatgggttaactaaggggagtcgaatcgtattgatacttatgaatggctatgttacactcaggttgtactggatttcgtttgcgctacagcttagacctttcgctaaagatacacgccgcagtgtctgaaacagacgcacatttaaaccgctgggctgttaacgctcattctcgctgaactagtctgtcatttatcagtgacatcagcttatctccaatcctcataagaccgtcgacaggaaccctcaattccactcgtaacagtcccacgctgggttgcgtagtctgttgtaagaattcattcatggttgaaatggggctgatgactatgaggcggcatctattggtatggtttagtagacgatcagaggaagtctgtatagtcagggctcaatatgtatccacgtagtaatgttgcctgctaccgacacgatttagacaacgtcagcgtaattacgaacacgacctcggttccacgtgtcatcgtctagatggtccctttgttcgtaggcctccaagacctcagtaatatctaattcgagcttcaagtttgctagacgttgacttgacgtagcagataaatcgcactgtaatggaatgatacctgaatcccgttaacttccagcatggcacatacgatttttaaattacgcttaagataaagaagcagtgcggtctaatccaaagtgcacaagcatatcaaaactcaggtctggtttgtacgattatttggagcagattttcaagatagttatgccaatctctccataaccatatacagtgacggggaccctctatgatacgtcatctccgggacctactttgacgctggagtcttacagatggtgggaccatttgtgcttaagctacttttagtgcggtaggagccctccacaatatgattcaaacctaaagaagctaggagccctctcgaccctggtacttggcattggcttaaatttcacgtatacgccatagcagattagtttaatctccgattttcaaaatactagatagggagagttctataccacattaactcgccccgatgggagaacgcacaagagttagttttcgacgccgcgtaaaacaattcaacatggccctcgagtctgctactgtagtgcatgaaagctttcctagttgggctagtagcccaagattctggaaaaattcaagttagtcgacagatgtttccgccttacgagtaatttaaagaggttaccccgagaccgcaaagagtttagtgcatcttatgtgcattgtgttgttcgtcagggggctttgcacctaaacggtcttacgtacaagctcagttcgtggatacatgaaagtcttggagtcaagacctacaaatcgacgcgattctaagtctaatgtatccttacttcgggcgtattgtgatagtatcataacggttaagacagtttaggataaaccgcagagacaaaaaatctcgttcgtgtaactgagtatatagtgtacacttgtgcccgcaaatgcatattattgatcgagtaatttaacgtgtgcctccttggtagagggtttccctaacatactccttttcctgattacctcagtctcctgcttcaaccggtctccataagtgagaggttgtgtgtaccgcactttagaagagtagaggtttggcaaattttgggagcattagactagtcgaatttcatacttcttagtcgtctgggagaacgtaagacctgattaaacgcatgatacacgaagtcattcagttcttcagttaagaggttgcatcaaatagcactagcttaaatgtaaatcgtcttaagtccaactattatgcggcacttgatcaccatttcactcacctcatcactacgcttgatagtatgatctcatcgtgatggtacccagttgagatcagcgaggatctcctcataaatttacacattgttaaaaggtcccgcgc

SEQ ID NO: 52 - DNA, 2000 bp, synthetic construct
  misc_feature 1..2000: note = Description of Artificial Sequence: Synthetic polynucleotide
  source 1..2000: mol_type = other DNA
tagatctgctttgtgaatgccgaatttcagattgactgtccgcgcgctagctcattatgacccggcagttgaaatcgtatagggttggacccaactactaacggaactcaaccactcgccctgtacgagatcacagggaacgtcggctaaggaggttatggtggccttaccttagcactatataaagtgcgttcgaaacctcagtgattccccgatagtatgatttttaagttctaagattaaatttgatacatcagttggtcctagagttagtgctactaagcttaaatcaaccaaaattttacccgttctattcagaaggaaactatagtggtagcaagtgtgacagtaggtatagacttaaatagttacggcgaaatagaaagattacgacgttcagccttgtgtatcgaatttgtgactttagaggcacacagagtaatggacctatcatctacgtcctgtcagagtatcatgtgcatgattcgacagaaatctcaataataacccaaatcgggctctcttgcattgaataattcatcatcaacatgaggtaatagcaaaatgcctttacttcagttgattagggtgatggccgatcacctatgtatttgaacatatattgtatatccggtcggaatatggcatccttagccgtcgtgcgccggctttcggaatttgatctgtctctgtttagacgcgtaacctcaattcgccgcaaactagatcactattctaataatctcactaggaatctattcgacatgcgatctttgattataggattcagaatctaagaaattgctacgatggggtgtcatagcgatgtctatttgagtttctatagtgaattggccatttgttttggcatcatagatcgctgacacaatcattgtgtctttcatcgatctggagtacagttagaagagaagcgagggctggtaacatgcttatagattcttatacttactaccttagggtacactaacaatatttgacattataggtcgaccaaaaagatttctctatcaggtttagagacaaagtcgtcgacatatttctgtttgaactcttgaggatgcacgaaagtgtctatcggggtatcagtgagaaggcgtggcaagcattctctaggtgaattccaccctttttagtcctcgttagtaccccgtagaccgcggaacatcgagaagttattcgtaaacgtgtctatctgttctatgttaggagtaggtcattgaacaaattgagctttcaaatagattctagaatgtagcgcgtaagtatgtcccgatagcggttttcagtgtattagttgcatctaatgtaattgagatgaagaaaaccttggtcgaagagacatgcctaaagaagaaggctaagtgaaggcctttatatcacgtggttcatagcccattatataaaaatttatattggagatgtcccattggtattgatagatggttggtagctgtcagcagtgcgccctaggtaaaccagaagactccttaacagatcggtataattattcgaggtttccggctctagcattcagacatggaaggttctttctaagcggatatattgctcgaagcccgtgaacctttagaatcaacctttattatctctaaccatcttttttacgtttcacctttaacttacgcgaatcgattcacgactgccgaagtacaaacgatgactcagtgttggttttcgctacaacattgagctcagctctatagcgcggactacaagttctgcgtagattttgccaaaaaaagttgcgggtagccttattcatttaacgtatgactgggaggcgctcaaatctctcactgcacctattcgcagacgcaaattatggcgtcgaccccaaactttcaggtaaatagctcacaagattgaccattggcaagtttgaactagtgtcgtaacgtcctgaacaaatgtttttctagccgctcctgctaaccttatggacattttcctcttcacccctg

SEQ ID NO: 53 - DNA, 2000 bp, synthetic construct
  misc_feature 1..2000: note = Description of Artificial Sequence: Synthetic polynucleotide
  source 1..2000: mol_type = other DNA
aaactacagaagaacccaaaggctactcactccctttgctgtgttcagctcgctggctcgtcaagataacggactcatgtctgtgggcaaagcaatttattacagctatacctttgtggaaaagtctccttgtaaaattgttagcaatattgtttcgagttatatcgaatttaaggtttattgttattcgtgaccataaggagctaacatgatgcggtttaatgcgtatggaaaagcgatagtgtttttagtgagggaatgtagaagacctcgtttcaacccttaccatacccgagggtgtcttaatctgttattaaataaagagcagcaaaataaaaaaaaaatgcagtgtctatcaaattcccaaatttggctacgtcgttcactaccaattttcaaaataataagaagaagtatatggatccagtctgattgtctttccgatcagcaatataaagcaccaacgtcttataagagctaaatagtgatgattccatgcagtataattcaattcccctaaagctactgtcgataaacttcatataacatatgtacttggaccgtttggtttggacttgacaggctttaagcagtctgcatcatgagcctccttctagatgtgcaagcattccccagaggcggttcgcttcagcgtggtaaggaatgatctctgggtcggaggtagtgcagaatgaccacttatcctatctagtggtttactttatctaaaacaacaggggactagatcttattatacggccaaaactgaaatgaagatcatctcatgaatattctcttaacatgagaaatttccgttgtcaatttttaaatggattaatgtcataaaatctgggatatggcgagcttaacacaatgcccctagtttacgttaagaaacatttgatacatcaacaaaacgtaggatccgccccggttttttggaatccacttctagaagcaggagcgggtcgctgtatttaagtcataaaggacgtcgttttacgaacaagaccgtgtatgaatctggactgttacaacggcccatccccaccactagttatactagtcaccgaataatctgaactattttactagaaagtctagaaattcatcctttgacataaatggattggaattaaaaaaagaatttcaaatataatcatataaaagtggatgcaccagagctcatgcgacgtcattctacgagcgatttatagcttataccaataaaccccgcgtgtattaacggtccagtcaaaaatactatgataccgaacaaggtttatcgacttgtcccgttgaaatcctagatgaagtttataaccaaatggcgcccctttagtgacgctgtaaacgcagatttatcaaacaggaaacatttctgattaaccagaagtatgcgtagtgaaggtatatcgcgcagtaacattcaggtgcttcggggattcaaaaacgtgttgctggtatagctcgcctgttttatcgaatgtagtctcaaaatctagccgagtttatcaactggtcgacgctggaagtctgcacttgaacatcgttcacatgtaagccagagataatggcctcagcatcgtcttattgctaatctcacgctgctttgtcgcgacgtactctctgcattaccaaatgggattagtttaatttcgttctctgggtgaccttgtgcacgctatgtgggtttgtattagttgattaaagagtccctttgaagatggcttcactcaccacatgactacacttcctatcgaggtaaggaaacgttttcttgtgcaaacaccccagacttaccaagtttaaagttttgtataatattaagaatttatctaacactgagacaccatacacagcttccgtaccctattggtccacaatataagacgttagatattgccaataaatgcttcattcggttttttgttagacaattggaaaatcttatacataacatataaacgtttcgcatccctggttccttccgataggtc

SEQ ID NO: 54 - DNA, 2000 bp, synthetic construct
  misc_feature 1..2000: note = Description of Artificial Sequence: Synthetic polynucleotide
  source 1..2000: mol_type = other DNA
tcgttttatcacgttttaacattgaatctttagtgcaaccaagagccacttctcctgggttataatcatcatctatttagcataccaacgcgtttggctgcctcggtttgtatatagtcgtaaaagcctccggtttatgaggtgatggaaattagttggatacttgaatagataatatcccatgcggtattcacccactgaatcacatcgcctgatgatccttgctgtttgcgggagagctcttctaatgatttttgcaaatgctgtgcatccctaatagtcttttacagggcaaagtacagggattgacagcccccgaatgtctacagccgacaaaccgaaagtcttctaccccgaggtagctgaaggtgcatagacgtagacatgttgactaatctcatcttgtctactatcttgtacacaaaatcaaaattacaattatatggaaggcatgggatgagtgatcgttaattagacaggggcgtctttggcaatgcattctcttatgataaaaggttgaccagattactgctcatgacttagtgtccaccggcccaacaattaataattaagagactcaaccgacatacgttaatacccaataatgccccaatacccagacttttacagggttattcgtgaacatgagtccctcgacatcttcccagattttaatccccatattactagtttgtaacagattggttatgggactgattagaacagggaatttcagctggaaatcactactaacttattgctagtttgccgatctaagaagagtctttgctaattgattttaaagagatattctgaacacgtcaatatccaaattttatccgcaccattctgacgtaatgacgcctagagaacgagttggtggcagtctatcgcttctgtttattttaaccttcaaaatatgataaggccccagttataaactattttttacggcaacttcggattaagtgttctatacgccaaaactattgatttacttaacatttcatcccgagaagctccgtcttatcaagtacgagatgatcccctattagaaaaaccacggctagtatcaacgacatgcgttacacacacgcctcagtgggggccgtcacacatagttcaaatattgatactgctcgtctcgatatgtgttcaatgtcggcaatcaagcagtgtcggaactgaacccgcactacgggctcgtaaacgacccaaaatcccctaatcaatcattgtagtaatggtagcaacttgtatgtcctgtcaacgcaacaccctcctggtgaattattctattagaactactaaaaaataaacccgaggtccagctctatcgtacacgacacgaaaacgtatcaaggtacagttcgatagccgtacttattatggtgactagcgccatatacaaggtcataagggaccttgttagcggtgtgttcacttcatcgtcagcgactcgttcgactgtcatttcaatgaaatctttaatgagtttaatagagtaggaagggacagtaagatattttatgaataatgtcgtacgtaggatttttttcaaatgatgactatcacagtacggcatacggaaaattcagtagggaattagatcaagtgtaaaattactggtatactagcgtatacctagtacgatgataattaacaatcacccccagcatgatgtgagaatagtaaagtatccatatttacaactaaaaagctcggaagctgaaatcccaaaccgcttgaacagctctcgaataataccggtgtttatcatcggaaggacagcgcctcaggattttcggcaaatcatagctcttatcttcgatctaagcgtttgatgaatattagaatcggactgagatataaagaatagtgatatatgtcggaaaacgacgatgtcattttagactatgatcttaagacggagaaagctaccatcataacaccgacttgtcctgccattgtattactggctttccatcgtgagggatagc

SEQ ID NO: 55 - DNA, 2000 bp, synthetic construct
  misc_feature 1..2000: note = Description of Artificial Sequence: Synthetic polynucleotide
  source 1..2000: mol_type = other DNA
attatgatcccaggcttcgttgagtctaatagctatccgactaatcaacttctcaggcatgtctcgactccgatcctggtggccttaaatttcttaggtgcacggaattgtgtgtacctggtatgtagagactataacgactcacttcttgccaattaggattcaaaactccctacttgagcaacgtgttcccccgcattatccatatcacaacagttgaatttttctaacgtcttctcctcaaaccggagggaagtgtgaatgtactgttgtccggccatgcctgaggtattttgattctagttagtaattacattaggaactcacttcgtcaactcaaacacgttgacaaatgtgcagttgggtaatacatgccgtgcaaagcatgtatgaccgtggtctactagatggcttcgcgatttactgttttgcgatataggcgtcggaataaacttcagcaggtgcggatgctgatctggcgccgtcatttataaagatatggctacgacttagctcgtgagatcgagacaaaatcaagatcttatcgtcttccacaaaaagtaccctcaatcggatattcggaccgtaaaaaagagcatggcgcttgattatcgtagctagcgcccaaggaacaattgtattattcagattaaaccccggattggacctattttcatcctagtagaaacggtgacgacgcgacttccgaaaactccaggaacagtgcggtctacccaggttgtagtagatgcccgttttctcagggcaaccagggcatcatacgttaacttaatcggttttaaccgcgaagttcgatacggactgatttaataataaacgcgaacaacctagtaatatcataaattgcggcgtgtacttcagaaatggtaactaaatgtcagacttcttgaaaaggaacaagcgcgctttctcaagtttgttgagtctcatcataatgggggaactccgtacatggtccgatggactcgatatccgaaggcgataataattatccccgtgttctacgctatttacgaactattaataatgatcggtcatgtcggtggtttattccattcctttatctccgataagtacgttaccatgggattacgcaacagctagattttcaaatgatcgggtcgaatccggcctaaacgaaacgtcgctagcgattgagaacggatgtacagatctctcgaatacatgagatgcgcgtaatcatagtgtacgatagaacctcatgttatcaacaggtgctatcttagtaaaatacatagtcatattctttacacgcgtaaagattctttgagccagcgaacatggaaatgggcgttggtgtgtttctccccggctttcgtaatagtcgccaccatccgcttgggtgctgattcgatcagttctaaccaaggagcctgacagtcttcgatttttgtgtattcctgtagaatatggcaccataattcagcgggaaaaaattgtcaactcagcagtgtctattaagagattactctcgcttttggactggtacagcctttacctagtaatatagacggacaaaaattttgtgagtcagacggcatatcctgaaaacaaatacaagtgtagtctacgttttagaatagactgagtggcgtcggtagaagttactgctcgagttattgtaaaattcttgccaagaacgaagttactccatatggaaaagatgactcaatcgagtcttactagattatttccgaagtcttaaacgtttagacctaacttagtcgaaagttgagctccagaagtcatctctcccagtttatcaatagtgggtggaacaaattcatcggctgttgaccttattgcatccacctcgttggagttatcttgccatgtatcctcaagtgttccgacctggaagtatgtagaaacccctttgaaatatctatcacaaagcaatatcttatattatcttcgtagtttttagaattatatctatttaagggcacaaagtctag

SEQ ID NO: 56 - DNA, 2000 bp, synthetic construct
  misc_feature 1..2000: note = Description of Artificial Sequence: Synthetic polynucleotide
  source 1..2000: mol_type = other DNA
ttaacaataaatgattaggttgtgcttgcctcctaattttgtttaaaaagttgttcttctgctgactagtttgattctactcatttctgtagtaccggttcggcgtactttttttagaggaaaatactaatgtgcggaggagggcttaagaaaactgcagatcactggatgagcaggaaaaccgaaggacgtgcacgaaaatcggacttgctgttgtgactatacgcaggctagaatcaataccgtcggtgctcgtgcctcagccgtatcagatatgattcttgagcgatgttatcgttggatcaaatagttcttttcgtggaaaggtatggttagatatccggggcctcttaatattggtttcgactagatctgacagagtcgggtcaaagctaacgctgtcgctaatgatgacagtgtcaatctggttaagtatactctggagttattagtcgatctctctcagtgtttcttaaggtgttctcagctggccgggttgtgcgcttgtgagggagcgatagcagtttgtgctcggtctacgcagtagatcgttcacaacttagtcagaccaatttatattcctatgcctaagaaatagtagatcatctaaatgtagttgccgatcaactcaaaaatcatgagcagtgataaacgctagtacggagctagcatatgcgcctgccgatagattgcatagaaccacagaatctctaaatttctggcactgactttaccttacttgtctactgatcatttagttctaaggcgggtcccagcatatactgagtaaaggaaattgcaacggtccaacaaagaatcaataagtaaatagaactcatcaatctccatggttttttaccctgtggtatgagagcttcgagacagtacaaatacattctacgagtgcatttattaaacacacggaccctatacaaattaatagcatcactagctcgaaacctattacagcctgaacgtttcgaacgcacttcggtatacagtgtactcgcgcgcgtgttgaaccgaaggtgctagccgaattagttggattcgtatatatgtgggatcccgatttccaagtccttgctggtttaacacacggatattagttgctattattagcgtgtttgaaaaccatgtcagagttaacgaccggctaaaaagccgacttataaaaagccgagtggtttggcaaccttctactggtcttggaattaacttctgaataaatacaaacatgaaaagagtgaactgctagactgcacctgtggaatgatccataacagttaaattactccgccgagtccattttgctgacggtggattatcctaactgaagagcgtacagcgattctgtccaaccgttgaaatcagtaattttctatacctactatcgtttgaccaaactcagggaagcatacctaaatatcatcaaggcgagaaacttttagacccatagttgtattatagtctaatttcaatgcacattctgttcaggcacagactgatattgaaagaggcccgcgactttgaaggtgggctaaatttatgcaataatggcacaccaatcaacacagtctagaacttaccaaaccaagcctagattcacctatctatttttgatccgactgtataacgtattgtaatacctcaagacataagacactcataacaatttaactttctcttattaggaggctcctctatgggattcgtcgtcgagttaaatgatttgaggttttatgtggactccgagcacgcccggtaagaatttctaggacttaggatacaatgcaactcagtggagtatgttcccccgtgtgatctatatgatagctgagtacgacaataggcatgcgattcagactatccgcttttaattaccaatgaatgtcacgacggagaacgttatgaaaggttttctctagcacgccctatcgctcttatatgcgaaatacattcctgcttgtgaatggccgggattgcttacacattagcct

SEQ ID NO: 57 - DNA, 2000 bp, synthetic construct
  misc_feature 1..2000: note = Description of Artificial Sequence: Synthetic polynucleotide
  source 1..2000: mol_type = other DNA
cttaagatttcagctagaatggttctggcgcgcctaagaaactaggttaagtcttcttttgcgcgttaaataaaaattttgtcggtagttcttaaatggtgcacgaagttgactgcatatatatatgaagcacctaagagctctatccccccttaaatgtcaagattggctaatataccaccccatacacatgattaacccggttaccttcgacaggtttggatctttaaatacaattagttgatcttcgctctggcagagctcgggttcgttcgtagtgtataaaatatctctacttgcaattatcgtttaacccctgcaagagcgtctattggtcttgctgttttcttacagttgtatgctcgccatgtataggcaggtaaacagactttgacaagggtgggcgagtcgcgtagaacctttccatgaaggcatttatttttgattatctctgatacctgggtgtgtataattggatgcaacgtcgcttgctaagacattcgagctcgaaattctaggattttgtctataccctttagaatcttcacttctataaatgactaaaaacatgggaaatgacaaattagcaagcggcgcttttttgaatcaatcactagatatatttctaaaacttagcaatgctttcatgaaaaccactaattttaattacatatttgtaaataacccgcatcaaacgcaagttgatgtcgcatcatatatatctccatagtcatttctattcaactggcatgttcggttaatcaaacaaacctgacaacattattggtctcatcaaaatttgctctattggcatccagaagattgaattttgagtgaccagtaatattaccctctgggactacttgtatcttttgtaaaagacgtataattgtagggaaaatttgaagttgtaaactagaacaatgaaataaatcacaagcctcttaaatttccgagtgtgtttaatagctgtccgaagaataaatatccagggaggatctgatctctaaaaaggaaactttcctaggtgcaattcatgggacaatagtctttaccatcatttggatcggaatctttaaagatttaacgtaaaactgtagatgggtgaagcaaccactggtgtcaggattgttgtaataacctacaatacgaaaacacatggaaatatttttttcacgagctatacacgtagttatacgtatgaaaacaaacaggactcaaataatctatagaggaatttataggttcttcgtgaacgtttcgagagcatagacatgattacaggctgcagatgattgctctagggacactggatacgtctgtctcagtatattaagaggcattaacttatagagctggtttgagttcctcatgagagagaatatatatttgcacaatgatactcaaaaacttaccgctctgcacaatccgcacatcgcgatcatacgcgccgttaaagttatcatccaatatactcataaatggtgtaacctagctcctaccacaaactgagtaccgggatcgctatccacatcgctgaaacaatgggaaaagaaaggtttccttcgagtcacgcactgactagatctacaatacttatgctctagaacgcgtgatatttctatgtaaagtaaagcatgctactaaggtacatctaattttacgaaaccgtatactactactcgccattggtatactttagactttgtaagtaaaaaacgagtagggcctcaaggacatagtcactgcttatacagcgaaacgaagctgctaacaaagctcagaccggtattgctgttagtatattcttgttagaagcgtacatcggttgggccgtatggtccgattaccttaagaatagttgactaggatcgtctctaaggtcgtacttacccacctagcagctgatatcttcgatgcctatatctgtataggtagagattcattctcagcgcattgccgcggtagatcctatgtagattatttagcatagttaatta

SEQ ID NO: 58 - DNA, 2000 bp, synthetic construct
  misc_feature 1..2000: note = Description of Artificial Sequence: Synthetic polynucleotide
  source 1..2000: mol_type = other DNA
gaaccttgggtccttatcctgaaataaaaagaaagtgcacgtctccgtaatatatggatgtctcagtgatatccacgattacatcaagctgagttatttttaatgatagttgactgtattgcctaaaacgtatctgtagtaatgaatacataaaggtactggtgattgagaagttctcattaaacgttaaaatccgcatcatctgtaaaaggtgggtaattgcactatagagggtagaccacgcctgtagcccgcttagaacaattcttgtactatcatttttaagtccttcaatgtctatcataagtattggacattgcacgagaaaacacgggacaaaatgctcgtcgtttgagactatggatcgctattcgggtcgagcaatctgaaacagatattgtcatgtttggaaggtgagcccattagtagtaagcgctttataccactattcaggagtaataatttaaggagtgtaacagtatgatgtctaccggtacacgggagattgtaatacagtagtagctccttatggcttgggaataaattacaaactgaacgctttctttagagctctagtgtcctgatttatgggtaaggcgtattatctgcaagtctcagttcgggataggtattccgtcatctaatattacctctagggtgtatactaccatcctttgcagactataaatactatctatcgtcggcactgatagatggaggattccttgcaagacctgatatctccgtctccatgtctagtttatagatttgccttacaagttcatttatgcatgtgtaatagaatgatttatatgaaccgtcatagttccattttagcatccgagcgtgtgtcctctctcgtaattaggcgtacgtcgaatcattttgctttcactgtaaataggcaaagcaaaatgtagcaaaggaaggaatgaaatgatcattctcatgctacatgtgtccttatacataaaaatatatatacttgattaattgcacatgaatcacttacattcgattatcataatacatcccccactcggattgctccacgaccagatggttaaaaagttgaatctgtgctttgatttttaagtgagcactcacgtagtatgaaaccgctagctcaggttttttttggggatcgttcagtattcacgaaagaagaatgcggcggggtggttccacaccatatcaactagtgtttatagttgcttatataacggcaaccggctagtaaatggtaacttaacagtaaaatgtctaggattagtaaacatatattatggaggcgttaaggctgtacgccttgatagtacacacctttttacaatcacaatcctaggttgatctaaaaccgttgacgtcaagtccattataaaatcttaatcgcctgatttccctgtcctaaaatgaagagattaaagaagtgaaatatatccctaagccagaagtgggagaataccatttggatatatgcgagcttctgccaaatcttagagatttctggacttttcaattatccaatatgaggcttgaggattaccaactctggactacatgacagttccacagaaactatttagttagacgcagagccaattagaacctcgacaattaggtaaagtaaagtttacaatactgttaagtcgcgtaaaaaaggttgattcaactatgacgggtatagaggaggaaatagaggctctcgttagctgtgtcgttggacatagtaactttttacaaagaatgttagagctgttgaatatttacgcttatacaaagtatctgctgtatcacgacggattttatccatgcagggcagtaatccatcaggcttttggagaggacagccttgggaaggatatcgtcacgaggcgtttcgcactcagacacccgaaaaaattacgaggaaatgataatcgtaacgtggcgcctagcgctggataattaccataatttaacagaggccacaacaggttttcacccttcaatgagtgtaa

SEQ ID NO: 59 - DNA, 2000 bp, synthetic construct
  misc_feature 1..2000: note = Description of Artificial Sequence: Synthetic polynucleotide
  source 1..2000: mol_type = other DNA
gattctgtacaattgtttcaaaatatagcttaacacatttgatggaataataagggttccaactagatatagttagttaggagttacgggagtggtgctcgggtacaccgaagcgtttatgtctaagctctcttctgagggggctcagacagctggtacaataattcatccgagccgcggtgaatgcggcatcaggccccttctatacttataaaagagcatatctaatttattggcatattcctgcaggctacataaagtcactcggtcgaggcatccctattcgggctaaatttcaacacgtctggtttgaatagcgactgttttttacagatggcttggataaccaatcaaccttcaagaagcacagttcttatgttaggaaccgtatgcaaccgtagactcctattttcacttgcgtgagcattcaacgaaattgggaagacagatggacttacattaacgtatcggactacgatcgtaatatccgtgatgtgagtattatagtatacaagagtgaggagatggaaatcatgacggttatcccacgtagcagcacacgcagatgcagaccagacagatacgaataaacttttttgtacggttgcccggtaaactagcctgggatcccgcgaacaaatgttagaataaaaacgcgagagacttgctttagtagcttttcatcaggattccttgcaaaaagttaacacaaagtaagcgtgttgttagtaatgtaatgtttgtgaggtaacactgtgggttaagtagtactaatgatctttctttgctgtttgactttcaaaatgcgtggagttcagtggtggcaaagattgtttaagtcttacgtattggtagtactcgttaagcttgaaagtttcgattatctctttttattccgatctgaaatgagcttgttctatccgaagctgaggtagtccacttagaccgatctatcgctaacgagaataatacttattatttaaatcctttctcatgccaatagaggagactgtcatggtaaccggtatgcttgtgttcatattaattctaagatttgctacaggattaagtctagttcaagtcctattccaaataccacaatctctaaggcctcacacgccttaacagaaaggggattatacgcgtcggttgttcgttatgccttatagtactcaacccataaatagatcgcacataagagtatgaatcggttgatgaaaaagtacataactcactacagtgccggatgagagattcccgtgaattaactagtggctacaaaacgtaacgtgcgaagagcaaaggtggccgcgatattacctttactttcggtgccttagtaaaagaggataatggcaaaatgaacgtcctgggcaatcagaccagagggaatatgcttagctattggctttgtaattgttgtagtttttaatggttctaaatatcaacaaataccatcatgatagttaccgatcagatgagcttgagccgttgaaaagaatgcaaatacaaaatcttgttcattaatccgatgcaacgtgccggcttgaaattcattttcgaagtagtgcgtccccgcgtatagacgctacagtagctccgaaggtctattgttagaacaacattttagaaacgggcctaataggagttcctcgggaaaaagaggaagggacaagttgattgtctattaagatagatgatcctattatagcgatgtcaatactacgcccagtgacaccatcaaaatagactggaaatgatggtacgattggatgagaagatcattagctgcctttaccttcgacgacttcgtcgtagtgagggttctgaccaatgtccatagcagttgaaagcgcgacattactcgaacaacgctgtggtcactctttaatgattcgtataatgaatcttcctctgcaacagttggacagaaaagtggcttcttgcttaggacctagctagactttgttgcctttctatgtaatacgtacgcaaattcc

SEQ ID NO: 60 - DNA, 2000 bp, synthetic construct
  misc_feature 1..2000: note = Description of Artificial Sequence: Synthetic polynucleotide
  source 1..2000: mol_type = other DNA
cagtagatgaggataagcccaagtatcgattccaggaagccgccatatggagatatagaggtatctctggcttcgcgaactcacaaaggagtgtctcgatggacctccataggtaacaaagatcaaggccccttaccaactcatgttctataaactgacatctatgcaataaagttaacaccagaaggtgggtcagaccacaaaccacaaccccgctcaattttagaacaaagtctactaagaggtgcgaatcaagccgaaaacgggagtttattgtccatatgatgctggatcggattattgtattataatagcctaagatcgtgtctccgatccaaatgcgtgtacgcatcaatcctgagagatccgggatggttgctggggttaataacttctcctttatatccggatgactgctaattcctcaaatgcaatcattctggaattatgaggcctattaaacgaatttaacagtacctagtcggtagaaacaattctaccccgcatccttaagtctactttcagagctactggcgcctttgacgcataggtaaaaccggcgactagaggaatgtcgtatcaagataagccctaatttacttatgctagcctgtgttcgataaataagatgtctgaattgaattcgcgcagaaaccagtgctgccacggtgaagagtgatcggggcggctatcaactacgcggtgaactaccccaaaacatttaggacatgcgaatatatcaaagagaaatcaattccattagttcgaagatgagcacgatcgttactaactgcagacaaagaaggcactattgatagaaccgattgacaacccgaacgtgtaccggagtttggatcagatcttgagactgcgcttaaaagcaagaacccatcacaaaaaggcaatagcattaggaggaatcgcgcacaagtacaataactttttccgtattttaataatattaattgtccttctcaccacgaggccgtttccttcgtggaaccagtcgtcctactttctctccgtaatttcattttatttagaataaaggtatatacggacgactatcgttcggaacaactaataacagtgcttggaggtgaatagaagtaagttgaactgagctaaagtgaacaactacaattcgtagccctgatttcattgtcattttttttctgactcaacaccccaaagatcgcgcaaagaataaggccatagctcaaacccgaaaaaatcttctaaggcctgataacttagttattatatgaacaccggtaatccctgcatgcagcatatatgaaataaaatgccgtcgttttcattgtttcgtataagtagggaacgaggtccatgtgctattttgctcttttatgtgtgcccaaggggtactggaatgtcgagtaatactcagtccttcaatgctcatcttgtgaccaaattcattggggaactccattgggaaaggaatctgtgagagtgaatccagactaggatctacccacattgtagtctgaattttaccttctagaaagtaccgctcaagttgactatattttacacaatgtgggctgatggctggtctccggttgaggaaggatcaatcatactcatcatgcatacatgaagatatactagtatgattaacaataggttttcaaaacagacactcgacttattgagcaccctattggctaagcaactgcatctgcactagcaatggatcttaaggcatcatataaccggttaggtactttcttgttaggtagaacaacacggttgatcaggccaatcgctactgaagtaatgaaatcaataaacactgagtcttatgaagtactattacaatctcctagggtcgtatcagacctttgttatgttttaaggacaatgcgggatctctcatccaaaaagcgaaattgataccaggcattggtagtcaagattaccgaattattttacgtaggtcattatatgcctgcaattttggcgctttacgctca

SEQ ID NO: 61 - DNA, 2000 bp, synthetic construct
  misc_feature 1..2000: note = Description of Artificial Sequence: Synthetic polynucleotide
  source 1..2000: mol_type = other DNA
gtttaatctccttgactaacaggagtctcttgccaacggatgtacgtaaccgtatgttaagacattatgaagagttaatattacatgcaaccattcgatttgccataaatgtaccgaacgccgttatatttacttactggatgaaagattcaagaatcaatataagttaaaatcttaaaaagatcaatcatacgtataaagtctatttgctattagagacgactgtctgatttgatgatgcagcgcgttgttataaacctcataaataagaggcggtggctttcttactattagcacaagtctcactgagtagtagaataactcttactctatatgtttcatcaggtacgaccccacgtggcaaaattacattttgcacacgaggcacattaagaccgaagagaacatttggccgagaggtatgtcaaagccggcttaatgatatcgacacaactcataaatggtgaaagttataaccaggtaatcttatgggattctgtggagtaaagcccattggacttcggaataaataagcaagctaatcagttataatagcatatatgttaataccaagcgtggaatgagcacattttggcagtttaacactaagcttgataaaactcgtagagtagcgattggacactacaagacgcgtgtttcgctagagacgaaccaccttgtgccaacagattactctgaagctcgcctatttgtggaagtaaatattacgtaacggttatagcattgttaacgatgattttgtcgagtaacggtatgaatttatgaaaaacgtcaaacaagcgtgatcagtttcgcatgatcgaattgagtttttgcccgcgcagggttcgcgtcaaaacaccttagagtaaatacttaagaggaatcgctacgtctatttgtaaaagtccgagtacccaccttggaatccccatttttttttttccagtcagctcaacggttgaatccacgtgtccgaagaagctctgagcaaactatggtgtcgccgttctaagcccatttcaaacgttatggagcgttgtgcctctttgttggcacttgttattcaccgcggcgaagtaacgcgctcgtcaagcgaatcattttatgcctactcgggctatagttaacggagttaaaatgcttcaagtgtaggtcgacaaaagatcaggaattcgagataaactctccatgtgaaatagcaagtttacgtcctcgtttttgattatagactaagattacgaattctttagcgctggctcatttgaatccaaaaccgtagaataagaaccccagacttatgtcctcgaaattatcaggtaagagaacaaataattcacgagtactgacagtataagcgcttatgtgagacgaccacgtaactacaatttataaacttgaccgttattatgtagtatttagtggctcataaaaccagcttagcttagatctgtgagactgaccagctgacccacaagacttttacattgaagttgcagctatatggaaacgtactttataatttcttaatgtaagaataaatttgctgtatcgctttgttcgtttgaactcttttctatgtaaaaggctgactaacccaggaagaggggagcatattttacaaattagtaagcgctctctcattcatttaatgatcaccttataccgacttcagcctatggaagatcttgcgctgttgcgtacctacagcgggtaaacggatgtgttaaacacgatagtaatagtaagtttccgttaggctgtagtttataacagtaacataagtgctaacgagatcaacacaattcaagttgcgaaagcaagaaaatcttgctacatatatcttagataagtatgaaaacatagattgcgtttttacaaaaagtacgaaaacattatattctcaagctcacgctccatgaacatgccatggatgcgagagctacttaatattatccggtaattattaaagtaactaccggttgcgcacaacggcttaa

SEQ ID NO: 62 - DNA, 2000 bp, synthetic construct
  misc_feature 1..2000: note = Description of Artificial Sequence: Synthetic polynucleotide
  source 1..2000: mol_type = other DNA
gactcttcttctcagtccacgtttgaaaatcagacaactacatattcaatggaagcgctgagtcggagtggctttccgattgactgcaggtgtctggcgatagattattaaaataaccgaggacctcatctgtgattacttatgttaacacgtcgttacaagcaaaatgtacagatcgtgtgtgggttaggggttcactagaatcggtggggcaaatttgccgcaaccgatatcgtatctgtcgccatttagtgggagctgggcgtgctatcagaatttatttaaacggtttggggacaaaagaggaccttatactggtagtataccttctttagtctttgctccgattgaatacaccggaacctaatttgtaaagaggcccagatgttggacagagtggttatgagtgcaggtttatagttcaagcatcagaatagtattaagataaaactgagggctttcaggccttgatttaaatgtgagagtattgtcaggccatttggaaatatcataaaatcctttgtgccagatagttatgaagctgcttagatccacttgccttcatttgagtctgctgactgccaattagagtcctcctcggtacgtatgaatagaaaacttcaaatacgattctccccaatttgctctgtgcagccttgccgatagtcctttatgtcatacactaggtgtgagctccaagggtcttggttccagccccgcaattcagataaacataagccccagtagcggaggagattttgaataccaaactaactttataacccgcgcatggccagtgccatagcgaatgcgcggggagaagtcattttagaagcctatcaggcgatcccggatcattaccctcgtataataaatagccttagctgcaagttcgtgtcgccgccaacgtattcggtatcagactctgatgtcctttaatagtgattatgacgactgtcataaactttgtagtagtgtatattatcgattgcgttttattcatcttgatgatgggatacatctgcacttttgagctaatctaagatcaaatatctattttcacgatcccgctactacggctcgagaaagttactttaccggaccgggcttaacacaagacttacgacgtcctggatagaattttaggggtttctaaattgatccggtttgagaacttcttacttatattccagtttcgaggactaggcatttcttcattaagaccgaggcatgggttatttttatattgtgatgcaaatcggtttgccccgccggagagactacatgccagttggtaacgtgacaaggcatgtgcaacgttctttagtgtcgctacgggattctgaagtctactgcttacctgattataccacggttcaacttcggttacaaaggatattcgctattgcacgggatggaaatttattcatgtcccaaaaaacaaactcgacaaaggtgcccacatgcggcctcattttacagtgcacttatgagctattgcgagctccctccaaatattggtgggacagttaataaaaacgatctgataaaaatagtaggtatcgagacctaagattggaatgatcacattcgcgtgttataagattggagatgttctaacttggatgaaaatgttagttacaataaccatatcctggttcgaagagtattgagatggactttcgacattataatatgatttcagaaaggtcgcacatgactgatcctttcctctgcaggtggtcctgtcatcgggtatgtttttttcctctagataaatggatattgtaagcaaatagtaattcctgcatgctggataccatacatgatgtgaccgccataagctaaccagcttctaaaaaaatacactccttgctagtatggtgattagttacggtgcatgaaaatagtaggaacgctgattctcgttcattttgtgtgcgttccacgacgaatttctgttcaaagtcctgcagatcttattgagacctttacagcac

SEQ ID NO: 63 - DNA, 2000 bp, synthetic construct
  misc_feature 1..2000: note = Description of Artificial Sequence: Synthetic polynucleotide
  source 1..2000: mol_type = other DNA
tcgtaggctaatagaaacagaattatcaattccttatttaatacatcactggactgagtcattctctcagagcaaaaggtaatcgcttcattaaggtattgtctatcctgtaagaacacccacgccgtggatatatctcaacatgtaattagggggtacatgcagtgtcgcaaaattcaagcgcgaactggggcatttctagttatgctagctaatctactcttgtaaaggagctttcgactaaaaactgccactataatctgattcaatggtggtaataagcggtaatctttaaccgtgtttttgctgtccgacttagtgaattgatacgtttatagggaaaaaataggtcgctcaatataccttaaagataatatcaccggcatgcgcctatgaggtatcgatcctgtgtctatgaggtaaaaaacgagactaaagtttgactgtattaataattatgaaagggaaccttgtagtcaaaagattaagagcaaacccgtctttcaatgacaagacatacattggatgcctcgaaattgattattaagtaaccagaaccaatgattatactaagagcttattcctttctccgcagactcttaagaaacaaggacaactgcccctgagcaaccagcctgctgatacgtccaaacaacccgttatcattagcctgtattgagctaaaagcacgtttattacttacatggcaagtattatttattatgtggctcgtataggtcgggtatagaaatgttgcacattacaagaaagttcaatcataaagcgaatcgtttatgttagcagactttatctacagttaacacgaggctagcgagatgtgctacttttcaagtgtttggaatgcatccgaggtcactataggcaattctttaccgcgatcaattcgtatttgaaacgcccggctagcctcccatagattcccagtcaaaggaatcaaggctgcgccattctgtgatttactccctctttggacaaccaacgtactagcctgcaggatacgatgccaacattaatttttataaccgtgagatcaacgcggtcaaggaaaaagttaggcataatatcgcggacaccctggcgtgaacgattaacatctgcgggatatgaacatttctcgatttactttaatgatacttggcttcataataaacataatacatccccctgaggttgataaacgttagaaacttaggcgagtccataagcgctttaaaggatcttttatcacacacgcgaaacattaccattcgataaaactcttatcactcatcccgaaatgccagtttcgcacatgcaaaaataagccttcgagattggtcacgcccgatcagtcgtctttcgctacctaacctatgataaaatagttcttaggagtcaggcaattgacttgcctgtgtctctttggaggcttccaagttcggatttaagggtatatgcctgttgtagtcggacaaatagataggataagcgctttccaggcggactacactattagtaactatcagcgaatataaatgtactcggcagcttaagcgtagacttagtactcgcaggacctcttgctcgttctagcatatatcctggtcgtttttaacattttaagctcgaaaaagttgtcggaagatgactccattagatggacgattaacgaacaaaggtctgtgaatgacatacacatctgatcagtattggccgcattcgcaggatagtacatcgcggggcagacgtattaaatcaacctctccacacccgggtttcgttttgccattgttgccctcgacagcagcgtttcattaataggaggctttataatacgtccagaaggtgtcagaggcctacgagctcacgaacgtatcctcataaacttattgtgtcaccagtcaagtcgtattttatctcctaaaacgacttacccacaccttatggaggcttagcgatcgtgtatatatgcttcttattatagtgcaccctgggttcta

SEQ ID NO: 64 - DNA, 2000 bp, synthetic construct
  misc_feature 1..2000: note = Description of Artificial Sequence: Synthetic polynucleotide
  source 1..2000: mol_type = other DNA
attgggcatttcgtcggacactaaatgaacattaaaggattgatcttagagtgctatattgaatcactcagcccagtccttcggacttccttgtatttcactgggcgtatactacattctcaaaataattttgcgagtcaattaaactagataccacctatggggggtttcgtcttggtttcaaattagatggtagtaagtttacgtgaacaccgttgagacgtagacggcttttatgggttgtctgtgttagactcattgagctgctcatccgaattattcattcagtactatttagcacttggacatccctgctagagctctgcgaaatgcggtattaggtctggggtgacctccagctcaattaatttacaccggtagtaaccaaaggttagttaaactcacgaaaatgatactcactgttttgtgtatccttagttatatgtcggcggattcaaccttcggataataagtaaatggtctcagatcgtagctgcaaaaaatcgtaaagcaactgttgttaagattggctactcctaacaaattccgcctccctcaagcaggacacttcggaatacaatccggaaatatggcgtgaaccctctatgatcgactgattccaatcacggttcagtccactctatctaattaacttatcgggtagatactagaaactcactcaaaccgtattcgtgaaataattattcggagtcagtaagcaaagcccagtgtgtattttacacttaattggctctctgtcaacttcttgcaaattaatccattacttgataataatatatcgcgttcaatggcaagaaatccaccgcagaatcgcaaatggactccctctcatctaggttaaagcaaaaatgttgagattccacctaaaagtggatatagaagacaaaattatttgtaccaacagtaaacagggacggaaggtgcctctcaggtagttactgaatacctgttagacgggttctgcccggcttctatgacttgagattatgtggttctacagtatatcatccgtctaggagtgaacctaatgaaaaatactctaggttggtacgtattcattcacataaacggatgcgatgagttggcgggttggaagttctgttaatgtcgtaagtacttataggctgacaagaggtaactgtcatacgaaaggattcggtctcgacggccgaactctaaaaggtctccttttccggagaacacaagactcttctgcttctgaccgtatttggatagatccatcggcggtacctttgtttgttggatcgtaacatctcttttgatcctactatgtgccaactcagttagttcgcgctgaattaagattcaagatcctgttcatatcttttataaaacatgtggatgtcttaaaactcatctcttcaaacgccattgctcgtttctggagtgttacgggttcggagtagagtggtattggatgtcaatatgtgaatttatccactctgacatacacaacgagtccgagaattttagatcgtgcctccaaacagcgctcaaatcttacaaatattaatgtagagccatggccccatgcagagatgttacattcgcatggatcaatctaagtttgtacaaaagaaaggcacttcttaatctgaacttcatatcgtgtttccctagcgattactatgattctagtgtagcgttagttgcttatgctctttatacactcgaggtatcatgtaccaacaacctagcgaaactgatactgagaggttgcagatagtcttcgacgatttagctactgtcatttaacattcctgcctaaaatagcttccgtccactcacgtactggatctcattctccgcgagccttatagagactggattacgtatattcaataataatctactctagaccaccgacctcatcccttgtttattgatagtggtgtccctagctgaccagtcttgttgggaagaagcatgtaacattcctattagcgccaacaacgcgt

SEQ ID NO: 65 - DNA, 2000 bp, synthetic construct
  misc_feature 1..2000: note = Description of Artificial Sequence: Synthetic polynucleotide
  source 1..2000: mol_type = other DNA
agagaacgtgtcacgtactaagtgcaaaagaggctgggttttttttgttagcttaaaacaccaatagacacaaatccatggagatttaaatgcaattattaatcttgatcgaattgtcttttagccgacaacctgttggtcccgacaataaatttaacgattgtttttatcctaagatcaaccgttgacgaacaaattaggcgaaagttatattagtagccagacgcgtttggaaacaggcaaaaactgctagaatacccgtagaaacctactggaataaatgaaccgatacgttaccgtctcaggaactacttaggtttgatagacagtggaatgccatatgtcttttagcgtaacaaccctaaaaccttattattggaaatttaccaggtaggatgtcatgtaacacgccaatccaattcatgtcacaaagtgattaggtatactagcatttataacttgggtaagtgcatctcatgtaagtaccgatgggcgtacctcttcgatgtattaaccagcacccacttcatacaagttcatcggtaagtggtttacaagaaacatcataaatagaaataacacctcttcagtgataagcggaaccccgtgccacttgaaacaatctctcgcagatgacccttggaacagggctgacagtttgaagtgacagggtgaagtcattcctttacaatttaagccgggaaatttatcaacactaaacgtaaaataaaattggcgtactgcctggacattggtcgcaatgtaatcttctttgttctcgtaaaccaaacaataatattttgaatcgtattatattgcacaggtaagccactgcaattaaattagagcccatcacttcccgggctaattgagactaagtcaaattatcctttcagacttctttaacctaaacatgaagagggttttggaattgttaaagacattccatggggtactgacgtagtaccagccagagttcgattcttacaattcacacgtataggtagagggtcccacagctacatatcctatcctgagccgaattctcgccattgttagctttaaatatttcgagccagacctgtggaatttagtgagttgaagactatgggagccataccgaagttgctaataaaattgtttctaattactcttcgtacatcagaggcacgccatgtgtgtgattaattcatcttgtttcccgtacaagcaatagcaatattgctcgcatcacgtccaccaagtaattattgtatagttactttgaactatatctctgtagcatttcgagtggtgctcagaggcgcggatcttgcctgtcggggattgtgaaagttggtcagaaagttacaacggtatggtattttagaaatcgcgaacctgattgcgtcctaacgcgatgttattagtattcaacggttggtcagagttatatacccctagagaggcctatggagatagacagtctcgcgtatctcatcataactcttgatcaatctagtcaagtagttcacgggactagccgtacacaataaggaacctaagtgcaaaaccactctttagataaggatcctgcgccatgctttgagccgcagcattctctcgatgagtccagcgtggtttgcaacacttagtacataagatagttaaatacagagcggtcctattttgaaaaagaaatcctatggaccgcaccagccggaggttacctaagacttcggacgaacatccttgtttaaatgtatgactggatgactgattttcaacagagcgaggtccaagaaaaactacaagccacttattaaagacatgagtaaggacgagttattgaaactaagacatacgtgggatagctaggtggcataatacaagcagataaccccgtacgattcaaacgatcttaacaagtattttattacaaacgggcctggttttaagagaaaaacgtgcagtaccctcaatatgagtaataagggaagtgacagggagcactcggcgat

SEQ ID NO: 66 - DNA, 2000 bp, synthetic construct
  misc_feature 1..2000: note = Description of Artificial Sequence: Synthetic polynucleotide
  source 1..2000: mol_type = other DNA
agggcttgcatatccacaaaaatgaatttatctaggttcaattacgtgttatccactccagcgaaaacttgacactaggattattgtcttttgtcgacacgttaatacagcaacgtccaagagatctcttgctttggcttgaacttgcaatattcacgggttgtttccattcttacctcgactggctagctgaatgacctttcacctgggttacgatgtacgcggggcactgtggcattaaacgaagtcattatctgcaccaacccttgataacaaaataaatatggtctgcgacaccttgtgctgggagacaaaaatcttctgtaattggttctgtacgacaggattagttcctctttatttcttaccatgtttcctcttccagcattaagatggtaaattgaatgtatagtgcgcgatacggagcacgtgtcagttgtcgctcggtcgtcgcgattattgcttggaggatcctaataaagctaaatgagtggagtagtagtatgcgtgtgtgccggccgtaatatctcattcacgtgcatcatagcgcatatattcgacacttgtaatcccgtctttcgaagaatctaggttaaatggatactactttttacacacgcatcctgcctctcggcgggaaatatgttattagaaacttctgaagttgtctggattaaagtactcatcatggctaaaacactctatttttggtgtgaatatagctctatttacttctatcgaggcctcgttctagaggttattagtgacagtccgtccgtaaattttcctgtatactcgtcttccttattagggttgaggtgtactgcatgtcttatgctatacaatcagcgtacgatcaagactgtaatatgtgtatacgaccacattatgaatgagggtaaggtgcgatagtcagtagctgcttgctattatccttaaatcgaataatgcagcgcttcaacaatagatcatatgtatttcaagcaacaattaggggattcaactagagatgctaatgtaggtttgtgaatattttggtcgtacattggtagggcatctgattgcatgtatacagtcataattcagagcgacgctctttttaaccttgggaaaggccgtgaacgaatgcgattaggccaatctagcgcatatagttaattattttactctttatctcttgagcaacagcggcaaggaaacctgggagttgctagacaccgagtagaaatcccttacttcgccagcggatcgatctgtactacatgcatcttctactaatggttgaaagtgaagctagtacttatttgcatggtgcacccattcttacaaccaggttgttctaatgtcttttcatcaattcttagcggagtgggcataatgaaagtataagaatggaagtgttctattttgcaaccggagaccacatgaaaggatcgacacagagatgcaaacagtgcatacattcgatgtggcatagaccaactcttgtacgatttaatgtgatctctgtcacaattcgtttaggtgtctatggtaaaacctcagccacaacatgtatagtcttacaggcatggctatcgtgatttaaccgtgaataacttgtcggtaacagaaactctggcacaggtgagcgtaatcaaatcaacttcagtaatgaggacttctaagatagttccgaatctgttcacagtattagcacggtgattgagttctcttctaatattcctatctttacattgcgtactgtcacagaatgctgttgcctctatgattttacaacggcaatctaaatcgtcgtatcatatgttcagaatattaaatagctcaactccgtgttgagtcctaagataaagatagaaacattgactataaaatctatccattgtaaaccagactaatcatgcaagcacaaattagagggcagaccgcggccattggaatcatttatatctttatcgtttaattcacaagaatggctaaatgccggattttgaccgggc

SEQ ID NO: 67 - DNA, 2000 bp, synthetic construct
  misc_feature 1..2000: note = Description of Artificial Sequence: Synthetic polynucleotide
  source 1..2000: mol_type = other DNA
ttacataacgactccgtcgaagccgtcccggacatcgagtctgacacttacaaccctgagagccgcttccctatatgtctatagattgcgagtgtatgccactgtcattgcagatttagggtcaccccaaaaacacgagtattattagagactacgaatcatttagcaaacaatttcgcgaagccctaattgaaaaggcaaccgattcacccctggatagataagctaaaatagtgttatgcggagcaatgttctcatttggacccatacactctattccttctgaatgaccttcgaaatacgaataagaacatggcgttcccaatcatccatatacccgttcaggctgagtagccaacatttcgtattcaaagatacagttgacaagctgacattcattgatgacttaggggctaacatatcaggccttttcttaatgtttaaatacttgcctattatgtggccatgaggagtgcgatgataccaatgttattggagtatcgttaaaaaaattcggtagtgttataattacgaactatagcttacgggtcatctattttaacatagtgagggcttcttcacacttccagtcgtcggtctgcatgaaacaaaaatgagttacatttagaggaatgcggggtaggcacaactaaacacaaggattaaattcgtcgcgacaggagtacactaaacgtaattaaaaagctaccaggcgaaacttctatttacgggcaattacgaatcctatgacacttcaaggacctctcattctaaaatagagacagcctccactcgagctccgattgagctctgctctcttccaaacaagaacctccgtgcgagcagcatatagcgagcattcttcggaaggacctatatagatcggtcagttgggaaatcttacaaaacgtcgagcatatattatttgccgtccgcaacctatgcacaggggcctttaaatcagtttatttaaaaaatctaatttcaaacagtcttgcaataggttaggtgggtatagagtatcaaaaatacgtgactaaaaacaacagaagttgataaacaacagtgattttcgggatttatgctacaccttagcgagaaacttctgttaacattgtctatgctttgaaactatgtaaaggaattcgtgatatggtatacctaataggcccataccattaaactgaatcatagtggacgagaagctttatcgccctctaatgcgtagtgacgaatgaaaatcagacaaccattatagaagtccgagtcagccacggatgttcggaattgctatatatacgcatgacttgccaaagttgtggtttactgtatatttcgtattccacaattacatatagctaaatctacgatcgcggcgcggtataagatttcaaactcggtaaacttgaatgatttaaatcatccaattgttttatggatcgtggcctggagtttggcaattaattaaaggatatttagctgaatgtgtaaaataatttttaacccaaatgtgtctataatatgtgctcggataaagctcaggcataaccacagatctacgcgaccttgtgatcgtccttgtatgtgtatatagagcaactaccaacagttgttcagacgcaatcaaacgatagctttacgataggatgttcatttattaccaagtactattattcactctatagggttattatatcctctactactccggggtgcgcaactttccttacgccattattaacggaatgagcggtaagcggcaccttctatatcatcgtcataagagtgagatgtaatgttactatgccttatgcttgccatggtaagccgaaaataagaagatcacaaaatagcaccatcttttccatagattctcataaacattgatgtttgagcaaaataacagctattacaatgatgtaaattattataaatgtctaatcataagccagtaatttcgttaagcaatctagagaagtatcttaagagcgttaagaacc

SEQ ID NO: 68 - DNA, 2000 bp, synthetic construct
  misc_feature 1..2000: note = Description of Artificial Sequence: Synthetic polynucleotide
  source 1..2000: mol_type = other DNA
cctcactgagaccaattatgacttttctcttgcaattacacaatagtgcgttaagtactgaaaaccatcctcaaggctaaatgttataagatttttcatacgagtggcgaaaaccaagtcaaactggttaaatgatgtctactacaagtttgggcttggctgacaaatttttctatgagctactgtaataatgcgtcttcatacgaacgcactctgcccataaataggcgatggacctaatacgtcaagcccatcttcaaatagtttttcttgtaaatttttgtcttgacagacatgatacgttaacgttgtctttgaccattatatcttcgcgatagggtcgagttcgtatttattaaattgatgaaattgcgacacatatcacgtgacttaatcccgaaaaattagagttcttgcgcttgtcataggcatgaaaagctcccctcataatacgtttgacctttaacgtatgtctttaacatatgttcctggtaaccaggatttaaagtcatggtcagccttcgaaaaatgtgagaagatcgcgaatacatcacgaactctctcaggcaaacatctcatccaccatttatatagtagatgcgctacccactgttaacctgtttgagatgtcgatttaaacgttagaaggtggttccatcgctggattgcaacctttacttaaggtcgatgatacgtacaatcgctttactttaagctaagttattggcatactactgaaattcacttcctggcagacttgcgttgctctcgcaatcccgcagtcctttatgatgtctaggcgttttacaaatcgacagtcattgtattaaagtcattggattgtacggtgtaagtcgacagggaacgtgttgagttaatagtaaaaggttcagattcttgcaagcgcgcttttctatcgcctggtttatcaaactcatggtgattatatattttgcaattcatcagccctcatatgttggtaagactcggattgggtcgacgccagactaacgtcataaatgttagaattattaaagacgcaattgtttatgatactcactaatgggtcgttagatacttattgttttaaggcaccagcctccatttgtccgagtccaggcccgagcttgggcgcaaaacttttagtatctaactgtgagtgacaacctttagagttctctcgtatagaaggtccgacgtcagagtatcataacctactggaattggccgggttcgcgtgcactctcacttcctgccagaacgcaattaagcatgctggtagtctcgacccggtacctcactctatcaaatgaaactatagtatacctatcgatcttaagatgtgggttctagctgtgactgcccgaagaaatagtatttcaacgacccgatcgtctaggagcgttgtgggagggttcaatgctctcgtatcgattcccaagacgttgtggacatactagctggcgaataatactatgtgtagtgaagtttgcggtaatctgcgtagtggctaattaagaaacaccgagccgtgtcttttgcaaactcatcgaggcgttgactaaaatgtctaacggttagggcgatattttatttttacccgcggtttattatctatgagtactccccattcccatatagcgtgcatagtttacttttccatatgttattagcaggctgtccgcccaaacgttgcgctagccaccgttagatcacagtcatattatcataacgattaccaggttatagtttcactgactaaggagcccataaatgttcattttcactagacatgctatgggtttggcccgaccaagattgataaactgcggtaatggcgatatgattaaacgattaaacttttaactaccatggggagacaagacttcttaactagtcggtatggattgctgcttgtaaagctaaacaagctgaatgtaagaacaggctggccggttcataacactatcacgagtggctgacagagttttacttatagt

SEQ ID NO: 69 - DNA, 2000 bp, synthetic construct
  misc_feature 1..2000: note = Description of Artificial Sequence: Synthetic polynucleotide
  source 1..2000: mol_type = other DNA
attcgcattgtttgagtagccgagcactagtgggatcatttaccttctcgcggaagagttacaaaagtactgaggaaatatgtgaattgttatagcttttaggaaagtaaacatgaaacaaggtagaacagatgacgacgtgatacaattatttacacaactggaaaattccgtcaaagttttaaagtatattccttgagtcctattattgaatattcgaaaggtagtcacctgagttgtcccgtaataattacataagtatccgtatggcaacaaatatctcctagatccgggccgcggatagttttcgctaaagtatctaaatcgaacttcttagcatacgattactagactatcaccttgagtagtctatatctctgcgagtgtaaaatgcacacgccgttaaatcgcctaaatgcctttccgtggccattatatgccccacttgctttcaattcattccataaactatgatcatggacccggttgcgagatgttacagataaagtcgaaactttcaagagcagctgacgacaggtaaaattacgatgcactgcggtgtaaggaaataatctccaggttgcaatagacatttaaattgtagaggaatagagttacgcaaaccaagcccaaggatctaccgaacccctctaccttatacaaactcgtcagccgaaatataccaaatagcacgttgcctagaggtttacattaatcattttacacgatccctttactattaatatatcgattccgatctaaaaggcgtttcaaggatagcaatagtcctatcaaaatcattcagttactggcaatccaaccaattcgctgtacacgacggggtgaggtcgtaaaatattatatgtcatagatgcactgtttgcgaccatgtctagcatttttcaatagctccacccacgcgttggcgacccattgttattcaaaaatgggccgcatgaagagttaattcgtcttgttctgacataagtgttgaccatcagacaatagacgtataccgctggttacctctaatcgaagatccagagctccttatgcaacgtatagtaaacctggctcggaaaggggttactcttatttttagcacctacattcgggatcaaatcatatgcactttcaagatggtgctcactataacacaataacttgggtttccagttaggatgaggaatccgccaggttactctatgaagtcaagctcttccgtagtttaggcgacgcttgacccgcgttcctcacaagtaacgcgacagattggagcaatagcgactgcttcaccatatagggacttacatacagatcgaatgatttgcagctttaacaacccataacgatctgcactagatgcgatgagatctctgtaaaacgaaacttggaattacccagagcagttctaattaagctttttcgataatattacacagcaactaaatgagcacgtatgctcaagtgtcgcaaaatccttattgtataggaataggtcgttgtcacaacataggtctgtcaccaaactcagacattatagtactttacggagcatgtttagacataatctgcacaatgctgattagtctcagtgtggtcaaattctttaacgtctctgttccaatcaaagtgagcagactgattgcatcacaactccatcacttaaccaattattaatagtccacacaattcattcactcttcactgttcagcactcagtcatgctctggatattccatatttccccgccacatatactgagtttggtcactcatatgttcgctaaaatcgatttttaagccattcttgcctattaacgacggtcctaatcgtttcccttcaccatggatatacggtacgggccctattatctgcgttacgcaatgtcaataaaagatattctaagaagaaaaaaagataagttgcgtaagcgtgctgcaagagacactctctcttcgcagtaaactaatttttcctttaagaatacaaagcgaaca

SEQ ID NO: 70 - DNA, 2000 bp, synthetic construct
  misc_feature 1..2000: note = Description of Artificial Sequence: Synthetic polynucleotide
  source 1..2000: mol_type = other DNA
ggattagattgtgccataacgcaacaggtaaaattattagaccagcaaaagaatcctaacgtatacaattttatcgtacataacccgtgaatcttattaaacccagccaggccgccttactttgctccaagtaggagcataatgcatagaagtttcagtatcctgtctaaagctattaagtcgaaatgagacaaaagtgacgagttattaacgatcagaaactagtctaaagggaaccctcctgcggccatttcttgaggacttacgtgcaccatatcatgaggtcctactgtgggaaaggaaatcctcagtttacatgatttgaaatactgtagtgacctgtcaatttactgatttctatgcataaaatgacaatctcaccgagtacgcataaatcagcgcagatctcatatattcataataatctccgggacgttattaaattaatttttttctagacagatattcagaagtccgacgttatacaagtgcccagtaacatgttctgagcaaatagattgtcgacagccccaattaaccacctactagtctttaggcactgtgtgaatgaagctattaagtactagacataatgtcattgctggctctagctgaagagtatacctagctttttttccagatttttgagtacgggatctgttcttgttgaacaaataatctggatggcgccatacaggcgtcgcctggagcgtcaagctcacataccctatcgtcaaagtatgttccgtcaaaggtgtctcagcacttaaatacttaaacaatccgagtttcgagttctaaatggttgcacaatatgcctggtagattgatataatcttgaagcaacgatggatgaacaaaaattattgatacttacttttacccacacaaaccgtctgagtgtctttttaagagggttacgaatatataaaagcggatcacgatattccaccgggaatagcgcaattagtcatatggaacatggtgtgaaaccacaactatgaaatctatccgtacaccaaccaagagacctaaaagttttacataatccgtttgctttcgtattgccctctatctaatgaaaacccattgacaattataaagaacaaaggttatcacacgctgcgtatttagagaagagaggacatgtgggatcaatgtggtcgcaaaaattatcactttaatcaacaccgattctaagaagaaataaacgtcgtattcaagggtactgtataggtacgttaagcgttgtcgtacactcagcgatttaactaacagccgggagaatgcataattatgataaagtgaatccacttagcgtctcgaatagaggctatttcgcttgcaatcaaatgcttaagagtatcctaaccaattttagacaaatatcagtatgtttatcgattaagctggacaattcctctacacagatgtttaagcgaactagcattttcatcctcccgactcataggagtccttcgttgcacagtagatagtcagcgtgtgttctcttctccaattgatatgctgaaaaactataggttacccgtttcggtcggataaagaatttgacttaattttcttgccgatagtaggtatactgtaaggcagccaatataaccgttagagcttgattagtatgatattcgctccttttaatgtatctacatctagctctggaaaacccggtgtagaagtaatgtattaagtctgcgaagcgggaatctgcttgtgacaaagattctgtcgcccgcaaacgtcaagtaataaatcgcagatacggtcagaaattccttctgcatttcaagattagtaatctattcgattccaaacatcctgctcctaacagaatgcgcacgggacctaatgaacttttcatatacgtttcatcaagcagtagtgttcggaaacgagacataacagggtacatgtgcatcaacctttaaaaaccaatctctatttggtatagtcgtattcgaaatccagtagtgaggtgaaaa

SEQ ID NO: 71 - DNA, 2000 bp, synthetic construct
  misc_feature 1..2000: note = Description of Artificial Sequence: Synthetic polynucleotide
  source 1..2000: mol_type = other DNA
aattggagccaaccataaattggatggtagttccaaaattttataacctattctagtgtctgcaagtatttaggagataggtgaattacacgtcgtacacataaatatgataatgcgatcaagagtgaatggggtctatagtaatatgatgtaaaacttaaggatattgtggactgatttaacgttacgtagtcctgacaagagtttagatgccaggtcgtagaagttgtgtatccccctattctcccaatggtagataccgtgataaaagataaattcctgttaaggaagtcgaggatgttctgtggagtgcagagttctacatgtgatgagataacctaagagaaaaagtaatttatagattgcccccgttaggagctacacccgactatttgtttcgttaagatatttgttcgtaccatgctgttataacgacactccctcgaatcttattttatggcaattaaagatgttacaggtggcgttggcaattctggtaaactccgcactttacaaattgttgtttgcaactctctcatattgtatgcaatcgaccccaaaccctcatcctcgaccctatgaatgaaggttttctgtgccaaaagccattttactcaaaaattagcttttaatttggggagcttaatagcgaattccagaatcgtttcatggggattaggagatatattataggagtccaccaatagtctattgacttagtggttttggctcatgcacggtggacaaaacttcaggcgtgttatctaattacaacccgtattcatacatatcaggggtgttgatttcagagaatagattaggaaactacgagcaataccaattttgaagatatggtctactagtagctcacttactcaacattgctactttattcgaaggcccatattgaggaatactgtcttgttgagtaaaacgatacccgtaactttaaactataaaggcataccagaaaaagtgtcaccgcaggaaaatataagaacgtccatcaatatatgatgcaaactagagaaagagcttgataaattatcaaactagcacttctgggaatactccgtggttgcaaggttacagggttcagtcaaagagttattaaatcgattgatatacttattcaagtgattgattctatatagctacgcatatctgctgactttttcgaaacgttgcctggttgtccagagcatgttttggacgagaaatttcgcgcagatatcatgattacgattggcaactaaggatgactagcgtaatgagaacctggctaattttgtgtttcttattcaaattgtataactaggtaaggaacgactcgttcagaatgagttctaatcataatcttctaaaatactgacagaaataataatatatattatgactattcagaaaacctataaaaagcactccgtagaagctcttcaatcttagaatcctcacctaggaacctgaagattattgtattgacttattttgtagttattaaagaaatccaacgacggggacgactgcttgtatgtaatatttccgttccacaagccgggagtaataataagcaaccgtagaggagcaatgggtttttatctcacgcacaggatgtcggagtagcgagccgtctgagtatgttatcaccaaagatatatgtaatatggttaatcagctgatttaaagagaacttcatcccaacctcgaccgacgatccgattactgtttatcgtcataccttacgagatgtcaggtcctcgcacaaaccgccacaaattccttgtcactgcaagaataagtttgtccgcaaactgtctacgcgctaggtcgttgtatgtattgatgagccctatccttatgacactcggactgctagccttctgagatttacgacaggcagtctagtattaaacccttactactttttgctgtatattgcattgcaagttccaacaagttaatgaaacacaaaccgtgatcgcctcaccccacaaaaggct

SEQ ID NO: 72 - DNA, 2000 bp, synthetic construct
  misc_feature 1..2000: note = Description of Artificial Sequence: Synthetic polynucleotide
  source 1..2000: mol_type = other DNA
gtaagggtcgaacctctgatcatattcgattactaataactccagatatatagaattgagaaaggcaaatgtattttaaacagcaagaaactgtttcaattcggcttatctgatgtacatttaataaatagaatgaagatcgagtattagaactgatatgaaagttcgtaacatcaggacgattagagtttatgcatgctaacaggaactgacctgctgacattatatcatacaatttcctgcgtcccgcttatggatggcgtcaataggctagtaacctaattgcagcttagaataaggagaaccaagtaacgacaacaaaatgaaaagcaatagatggcggactgcgctttaattgcattgaaatactctgggcttcaagtgttagttcattaaagctgtctcgcgatacacaaacgctgcgaagtggttccggagtaaatgtgaccaatgttagacagtgggcccgccatgaatgtgaagttagttactaggaagagtattctcagtttggtgtttactagaggtgtgcttggcgtttatctgggataataattgtaactcaattctattctttttcgttttttctgctcatatcgaagttttgctcgcctcaatcaacgttgtttgtatagcacttaggatcactctgcgcatagggaatgcttaaatcagggagttcatcggtgtccatcctgcagggacatgaaagctgtcatacacggactcgtaccggtctgacaatccgctttgcctcatagcaactattgagccgcattcgcgtggagctgaactatcagaatggctagaaaggataaacctgtggtgggtccacgagattggtcttcttatgttaatattagctcacaaagtccagagttagtatccatctcttccagtcacatggaattttactaattattgtggtatcattattataaaaatgacattatctagcatgactccctaccactagtgcagagctactatgtacataactcgctgtttatgcgatactccaacaagtagatacggtaatttcgatataggatgaaaaaaccttcataacagcttaagtttaacttcgagggtccgtgtaatcggacaacgcacatacgaagtggcacgacctttcatttgggctcccttttgcaggctagtaaacctagtatacatgaaagccgtcttgcttgtgcctacggcttatttcgttgaacgtacgtctaatagtgccaaggaacgaacacacggctagatcataatattactccaggtgatggtttcggtatttgcaaagtaaagataagttatctgattcacaacaatcgagaatttgtcctgtttgaacgccgaaatattatcttactattgctttactcagatacctccaataaattataaaatggcttgtttgaatgtgtatcgaaaccgaaagctatatcttttgaccgaattaaccaaatgctacgcgtttgctgtttattatgtccatcatcgctttaggttaagcttaataggttagggaaaactaccagcattcacataatatcctatctaggaagttaaattcacccatgtatactatactacttagtctacaatatttctgctttattctttatttccattatcaaagtatttcggctcttaaatggggcaattacgaaagatatgattctagctcatgctcaattgagatgaatttatgactttaatggggtgtaccatttaataatgcagcgctaacataacgtgcgacgctaatatcatttactaatagattttcattcactataataattaataatcttctggccccatggcacaggcaattttaaatccgtacccgtcagccctaaaatgccaagattagtgaatctggtgtcatacaggactaacaggtgcaaaaaccggttgcgtcatcaaaacgcaggatttactcaggatcttaagaaatctaaattttcgcagaatcgctcatcgccaaaattttaggcgtc

SEQ ID NO: 73 - DNA, 2000 bp, synthetic construct
  misc_feature 1..2000: note = Description of Artificial Sequence: Synthetic polynucleotide
  source 1..2000: mol_type = other DNA
cacgtggttttcagcggttaacgcaatctgcattattggtagaattttacacttaacaaaatatcaccacgcggacaactgatttagcaaatgccgtccgtgacgcgggacccgcagcacattattagacatagtacatcagcctgtaaccgatcagtcatcacatatcccggaaagatttcaatccagttgtaatcaacgcgtaaagttatataatcacttcaatcaccttactaacttcagaatggcagcctaaaaatctgatgctacgaaccgcatggtgttgaataaattcaatagaatggagctcctggatatttcacgacgccgggacagaaatagtgttatagagaagaaggcatgccgttttactcgattcgtaagtagtttgacgaagcaaaaacttggggaagaacttatgagttagccacgacaactaccgggaggatttgcttttcttcctccatgccaatcttggagggagtacctcaatcacacgatgaatcagccttaatgggcgcccaaaacattcttggtgccagaaaagcggatgcttcctcgaatgtgtaatcagaaaagtggtagatgaatctccggctccatcatggatagagctgcaggtattggtgcagcaggaacgaaggttctaccagtaagtaaagtttgacgttagttacgagtctagaaggcccaaagggcaaccaaaacgtcggcaccataacatctacaggtggtaggctaatgtaaaagtggttataattgctaggcagaaataaggccgttcattgggcatgtgtacactccattgatggagcttaattcctctcaaaataattacattctgttaacaagaaataacttattggtcgatctacgagctagcaataaataatcatgaccaaagagctgtgctgtgatcagaagttatgacgcttatacagagagcattgtaaagggcaggccgaagcaaattcacagagtacctgaagcgaacaaaggaagagacttctttataatttacatcgcttggcaattaaagaagcgaaacacagttgctcgaatcacatccttacgtgtcgtcgacaatatcataagcattactagtttagagaggtgagatatcggtagtaggtattagaacattctaatacctaaagctcattactattagcacctttcctcaccttatttggatttcccgcacgccgttcgcaccgagctaagtgcaataagccatggcgatgacttagatgtcacattgccccatgaattcaccccagtgagttgagacgatttgaagtttaatacgtcgttcgtggacagcttgaatgtttcacacgtggtaagttgcatatgaacatataggaggggccacaaagcttatgcgtgaagcaaatatgattcctccctcgatccgttaattagagttgctgaagggcataaactttagcgagtttgtattaacatagtcatatgaagtaacagagacccgtcataacgcttgaaaacctgaactcagaatgcgctttgtgtaccataggcatataccccacattacggagatgataatcgacaaatgctccaagaagtagacctctagccatcatcacgtgtctctactgtattctccgaagttccggaggccagttcttaagtaggcacagaacacacgatggatttcctagggacgtacgtatgttcgacttctcgtcagtaatcgcgacagaaatgggaaggtgagcttaacctaacccacatttttgtcatgggactctgtgaatggtgtttcttatgaagctatcacggtgtaaagatatctagacacgctatgtgctactccgataaccctacgtttaggtttacgagattggagaaatatactttattaattcttccctggaatcgtaccaacaagttccaaaatggctctgcggtctgtcaaaatatgaagggctcaacttgacaggacgactgaccggaaatgatttaagtgaacctcc

SEQ ID NO: 74 - DNA, 2000 bp, synthetic construct
  misc_feature 1..2000: note = Description of Artificial Sequence: Synthetic polynucleotide
  source 1..2000: mol_type = other DNA
ataattatcgacatagatgtgcttcactcgatttgacagctggatagtaagaattagtgtataacccaatacgtatgctaatacaaaccctggactgatttgaatgtaatcctattcataatattttagctaccgtaaatgtattctgcaattgaatttcgtgtgaatgtaaaaggtttagaagtttcctaagttatcgggtgacgtttttaatgggtcttaccgtagattcagacaatcttttggaaaccaactgaagaaggaaatcacacgacctggcggataagggtttgtaattcgcgttaaaaaactgacgtttgctataagagacgttaatgtaaatgtaacgctttaaattctctgtgcgagagttttttaaatgagatcaaggattgttaatttcaggaagctccgttattggattttgccttctcattcgtcactatccctctccgatcaatccgattgagtcctagtgtagaaagttcacatagaaagcagttttccgattagtctagcggggtactaagtgaacactagtcagttggtgatatactatagctaggctgtgataatgttaatcggtttgtgcctactggaatgcttaatttcatcttgaggacttgcgctaggaatcggtatgtcttcgttaagtccaaagtgccttttcgacagatgttggattgatgcactcctccgaaaaggaatcaaattgggtttataaattttgtctttgtgacacctgccgaatttagatctcaccattatccacaataaccctattatctttacctacttccgtcggagcttgattatgaatattggcagaattatgtaatagtcattaatatgttgaataaagatatcaatacattcagacaattgaattaatcctgcgtaaaaacctacttaggacgagttgctggtatttgtttttataatggtagacatgagggacatattacgaacctctgtaagcctgttctgatgtggccggcgatcacgttacctgatgagatttatagatctcaagtcggatgtcctctttaataaactgaaaaattgacgactaagtgggctaattatgccatcagaaataagctaaccaaacctctaaagtcgaccctgtagtataactggcagtgctagatatcacagggtgtttgtctactgaaatttcggcattctggtcacacttattgccgataggttctagtagctagtttatctagactccaattgaaagcttacttcggcctatcaggttgaatgatagacggtctgtcttaagaaactacaggacatatactgcatcgaatgcgtttaaatcctaacgcagaagggttgttatctgatcatcagtaagcaccaatctgcatgattacagacgtaccaacaactgaatacatcctgcctcctgagaactagaacctattgtattgcggatgagggtaagataggtagaaacctgctgccaacttatcgataataattatgaaccatgcgtgggtgttgatatagacttaatatgacctcctgtctggttcatataccagttttcaatgcttaagagaactagcttgtacggagtttttttaatacaagtgctaaattaacaattgttcaaaaacagtttatagtagtaaggtattgtaccaatcgtatagcaataaatcatacctgtgtttactccatactttcttgattatcgggcacgagaagaggacaactcccaaacatcaatgtagccatagtgaatgaaaaaagtcggttatgaatcgttagctaaatcgtttgctccaattaacaaaactataacctaaactggtgaacacatagataaatgccaactcgttatcgtgttatgctatagatccgaatttggtggttctccgagtctgtatcgtttttaatcgagatcttaccttattcctaaccacatttcgtaagcctattgaaacgggtattgccggttcgcccatctggtagtacgtaaacga

SEQ ID NO: 75 - DNA, 2000 bp, synthetic construct
  misc_feature 1..2000: note = Description of Artificial Sequence: Synthetic polynucleotide
  source 1..2000: mol_type = other DNA
gaggttagtgatcaagcgcattagctttttactgcggaacgcatacaggatatttacgcttaaaaaggtggatttcgtatttattaagtattctctttactgaattattgtccatcagtaatcgctggctttatgaactatcaacattcggtgttgtgttaagttattaatgacacatgctcgacgttccccaattcccgtgcgtgatatattatcatatgaccattaaatgattaaaggggcataatattttgaaataacactattaatttgaaacttttgtccttttcgcactacatgttggtaacatcgcacgcactaaatactgacatatcgtgcaccatgctttctaatagcactccgttccagtccatagctgagactgtcttttcggacaacacaatagataagagtctatctctcatcaaaactgtaagaaaagctctaccataattggggccgaaacgtaatacgattattatgatatcgctcctgccgaggtcaaacaccatagcactcaaaaatggtatccaatttagaggggctatgagtagttaaaaaataggaattaaggtggcaacaggacagaagtcaataggttcccttgaaggctagattaacagaactgtaatgtgactgcctgtaagcgcactggagacatcaagtattgtacgagtataattgcactttggaggtacaacatcgcactcgactctttcatcgatattttttcgtgggtgaacttgagttaaagttgatggtcccattcacaacgagcggttttcgcgatgtaaacgccggccaaagacaacctaacgccgaattattctacttcatatgcctaagtaagcccgttctttggagaagtctcatcctctattattatacatagttatcatattagtctagtcgccaaagtgtggtttctaattgataaatataataagttaaaaaatgagagctcaaagtttttccttaccgtgccgcacaagtaagtagtctcaaaaggaccgcgtagggagggaaaatttaatgagttctaatataatatgcaggcttgtgaaagctgacattgactactctggactggtcggatagttgctagacatacctattgtgacaaactgacccattatcgagtctagtagaaccggtccgtacaattacacattcttcgtaaactagttctataaagactaaaaaaatctatatcacttggagaattatggaagatgagtcaactccgaagtgtggtcaaaaatattacagattgtatcaaatcgaataggccgtaaacaaggggtatacgttcacagtacaaaataaatcaaagccttcaattatatcgagagattattacactaccgctgctcttgactagtcaaacgtacctctcattgacaacattcagcatgattattgctccatgtcaaagactccgtgttcccattagttttaaaggcataatttatctcttttcctcttggataacgagagataattagacaatgctagtttcaccaagcccgactcgataagtggcggttttagcctacccaatcgcctaaatatatcaaaaatgacttgtacgcgataatactgctcgggtagttaacggccaagtacacgctcacagaacaacggttgtaccgcttatctaattagggaatgtacggctctctcactaatatgcgattaatctattttgatttttatgcagagcatcctaagtgaaactctagatgccgccaatttttgtttatcatttcgccaaccgtgaattccaagatggcccgccaaagggcgtataaatcgagtatttacgaagtaataagttaattctaaaattctttaaatatgaagacaaacaatgaattgattatgatttccagatatttactttggtaccggattaaacccatttgaacgtcattcgatatcaaagtccgctaataagggtttcaattacaattcttcaggagaacacatcggtaaccttc

SEQ ID NO: 76 - DNA, 2000 bp, synthetic construct
  misc_feature 1..2000: note = Description of Artificial Sequence: Synthetic polynucleotide
  source 1..2000: mol_type = other DNA
gcgcaaaccagcaaattaggtttgaccttcaacaactgtaactcgatctgcagacgagtgagtaacaacagctactggtacaatttttttgtaccgcagcattcaggtattaccccttcacgctcagtacagaggtatcgggcatccgtataaaaaattgacttctttttacgatagtccaatagaccgttagcttctacttcatagtactaataataacctaatgcaatagtctggataacattcacgggacactgatactagaatcaactacgctgatgagcatgtccagactgacaatcggtcgacatgagaaggaatagaaaaaatcctaccctgttaattctggtcatgtttgctggtctctttcctactcggtgcttctcaaatgccacatattcgagcataatacctagttataggcataaacttattgttgctgcccatgttgagcattttttatatttaggccttttacgaatttctgtttctattactaaagatgtcagagtaataccaccttcagacagaatcacatgattaaaactatagaatcggcggtacaaagatgtatctcacctatagagtatgctgataaaatcatagaccctagacatactattcttatcgccccttagaaattattgtaggggttgcgattacaacgcatacggtatttgctatatgagcactcatggcttatgtgtacaatttattgatatatatatttagagctccggatcgggttacagaatcacttcacgacccagcaaatgctaatgatttaagcgtagtatattggctttgtgtccagttttcactacgggttcctttctatgtcctgataatctgtacaaccgacataccctgaattcatgccgcatatgtcgtgttaacagtgatctagggtccagtgatagggtcattttcgtatcgtcgcatctgtatcgattggaaaagaattatacagtccgattatcacttagaactacacgaggggacctcttatctgccctacctattggagttaaagttctaactgctcaatctcaagacggccgaagatggttttaaaatgacggtccacacatttacagacaaattggaatgcttagatatatcctactgttgatttttgtccaaaattagaggcgatgtaaccccactgaaagattgagcagtacagtaattctaacttgaaaaaataaatttttgggtatgctcaatctttaaggtgacctactaacaatatcctagatcccatacggtagttcgacagagatccaatacattctaatcgaacattagtaagttaaataatatagagctacatttctaagtaaatcgatgcttgaagatattggtagttcgcagaatttgcatccatcacaaacactagtctttacgtttgccaattgctaggtagagtagattacgagtcaatcagaagaccaaattttttgacccataggatacaacacgtagtcatgacaatcgcatatcgctagtatgttagatctaagaaaatagtctacttaaccgggtcatacatctcagctattaacgatattatgttgccttatgttagacacgtcaataagtagagcatgcatttctgcctcaaataacaaatttgttaatatgcaatgaatacctgagttgaatgaacccaaactaaactcagggtccttccatagcgagagcgctaggctaacatgagattctgacgtcttcgtgagttgacaggatcttgccaacaaattacatatttgaataggcatgtacgatccattatactatgagtgccagagaaaactctgctggccgaccgttttacggggggaaagtcaaatatgtagtaagtacgaattttcctgggagactatagttgctgaacgttcttattctcattttcttgaagttaaggatggtaaaacatactatacctatgtagatattctttggtagtataactattatagtagcgtagacgttatgtg

SEQ ID NO: 77 - DNA, 2000 bp, synthetic construct
  misc_feature 1..2000: note = Description of Artificial Sequence: Synthetic polynucleotide
  source 1..2000: mol_type = other DNA
gcctaaagacctctatattttaagctagcataaaggcaggagacgttctaacatcgcaccgagttcgactatgaagagaggtattatcaaccctgtctcccagttcacaccggttgcattatcatgacgtttttgatttgttttttttgagtaacgggttcattgtacgttcgatagagtactcgataaacgactcattccacgcaagcctattttgtaacttataactagacattagtctatggctactttcacacccgaacttacgaacaacgagtattttttttttggcaaaaacgtaacgttcgtatgtggcctaagtcattaaaagacaaatattgaagaaaaacccatgatttaataccgataggacattacaagggtcattagagataacaaataaattaggcttcttccaagagttatccgactagttgtgctccagatctgcgatactgatcgaatttatacctcattagacattcgtagtcattggtgttggacttgaagttctgtacaatcctcggtgatcactcttggacaacctgctgataaaacatgtctatcgtcagtccagtttgtataataaactaatgagacaatatacaaaacaatccgtggcactacatgttgtataccaacataaattctgaagacctatgattcttgtggccgaatagtcaacagattttacgatcactaataaccatatatctgttacttgtcttctcagataggagcggactagaaatactcacttatgttattcttacgttactgtgccagacgagaggtttttgcagactctatggtttgccggatcttgctaggaaaagggtaactggtgcctgattgcatgaactatgtggtatgactatagatgaagcatccgtcactgagctcttcgaagtcttttatgagacaagaatattctttgatagaatcatctatgtctcaatttaatcaagggaacggttgggtactaaatcgagttatcatgaggtcctatcggaatgcattgtatttgagcaatatctataactgtaggtactatggcggatatttattttccttgctgcgacttcatgtagcaagtcggcaattccccgcggttttacattttctgcttcgaggtattaaggccctaaagttgtatatattataaattaaagatctggattattaactcagtgcagagggcgtaatctgacgtggcgacatgtagatgaagcttgcccaaaagatatgagatcttaatatctataagaagtatgcctactgttaattttggggagaaatgctaccccggacaattatgcgattgtcaagcgaatatcttgattttatccttggaataggtatattacttcggttacaccagatatgaacctatctattacttcatattttactcaggcttggtcgggacctgtgttactttaaaggcattaaaacatacagcgtcgacaatcctcctaatcaatatcctcagaaggaatttactcgcaatagcgaactgagttttttgcctgtacaacggtcgtgcctactcaatcattgccgcatactaatctctatcatattgcctttacggggcgaccaaggaggaatcctatctaatcccagggcacctggaacacctgcggaacatgcttcaataataacatcgtataagtctatgtctgcgcttgtgacgtcatagtacttcttctagtgatatattacgccgttggattgggatcacgtttagaacgacactgtgaacttctatatgtactcttttctcacgatatgccgtcgagttttttatcgataataggcagtgttggagcgggacgtgtcattagtaataagtttttcctatcaatttcctgcgatacttgactcctttggggcaaacatagacgacggttggagtcaaggtgaaccaaaatagaagtacctgggtaaatgcttcataggcacttggacaagacattaagtcgacacactatgccttt

SEQ ID NO: 78 - DNA, 2000 bp, synthetic construct
  misc_feature 1..2000: note = Description of Artificial Sequence: Synthetic polynucleotide
  source 1..2000: mol_type = other DNA
aatgttcggtcccgggtaagctatcattctataaaagtcccaccccgcttatttaagattcacagcgccgcaatgacgcggaacagggttgtctatgatgacctaactacggcactttaggtatcatatattgagttgagcgaatggatctgctaggcttcccgtctatcggatgctttaatgcaggttaatggcccgattgaagtttatagtatatatatacactgtgatggtgtaactacgttacttcgttactgatcaattttcaaattatctcatttgttaggctacaactaggactaaagctcaagtaaccgatgcgaagaggccgagatggtataatcaacgggggtgtaatctaatatacgaatcatgctaggagagcagcttatcgtcaaaactctgttggccagattctaattactctttattgtatcttttttcatgtagattaaccgtgaagacagtagttcatgtacgttagtcaattattgagaacattagcttgaatggacgcgtgctcaaataataccccagtaatctaaaccatattgttaatcttttacaagacccaccaatgacctaatgagttcacctccacatacctgtcattaggtgaccttatttccacatttgtattaaatactaataactgaccatattgtgctgtggttctgtacacttgtatacctgttcggctaatactagtcagtgatttcatagcgaatataacatttgacaagactgtagcaacaagtttttggtatagggtttgttaaagcataccgcgcaggacgaccgtctcttacattaatttactcgttttaatctataattatccatataatcaactagtcctgagccaaatcttcaatttcccccgcgtttgagattgcttgatgaggcgaaataagaggcgaacggaactccaaaaaagagcgatcttttatcacgtccctccataacgctttataagtcattagtcggcatcgttacaaattaatgatagaccagaaagtacacagacgtgtcttttatcctgtaacgaccctaattcggcaccgtctactaaatgctttgccgtacgctctgatgattctatccagcgattacgtatatgttccggggtaactacctaaatctaatgcggccataggcccatactgatccgccgatttcgcgcactgctttacttatatacatcagtactactcgggcaaccggtaaataatttacaatagaagtttaagtgcagttacatgcttaagatatcgagagaacttgtgaaatacgtacactaggattttctcaaattcgtgacattacaaggtctggtttcgcgattctcttggactgatataatatgattgaaaaatgtagtagatatgatcctggataacatttttaaacaagtcttgggtgagctcggtaccttaaatccgatcatagaatacaacatggcacctacattcatattaaatagtctattacatgataagactccttcatgtctgaaacattggttagacaattcgcggtttcagtgggtagcgtgttctattgacttcgaaatgagaaagtgtttcggcgcgtacggtatatcttcccccatgattatacataacatccttctaaaaatcgcgccactgcagggtcctcttttcttatatattattgaggatttggaccgatcaaacttaatattaaatatgattctacatacaaaggtaatgatggcaatctacttgcgggctcgactcgtagtctgttcaatgaaaaatacatttctcaagaaataatcttcgagctatttcactctgtagttaaagtttcaatcttgttacatactgcttatacaaatttaatttaaaagcatgtgtcaatttaaggctaaatgctcagtgtaaattgtattggtaaactccctaagactaatgaataacttgataatgtggatagattaaatccgtgcaagcctatcctaaaatcaatttgaagtg

SEQ ID NO: 79 - DNA, 2000 bp, synthetic construct
  misc_feature 1..2000: note = Description of Artificial Sequence: Synthetic polynucleotide
  source 1..2000: mol_type = other DNA
tacaaattgtccacgggcgtgaaaacaagcccattcttcttcaattgcaagatttgcgatacttaaaccttactgatttaataatcgattcaaaacgcaagagtcatgaacagaacgagaccccgccatatttaaatgcacattcgtgcagcgatgggtatattgaggctgtgagaggctcaattaaacattttaccaggagatgggcaaaataatgcgtggggatcgcgggactataatctaatcagtcatactctaaagtgagcttcgtgatatcttgaggataaaaaagggcctaagcgcacagggttattgagttccagctaatgatgctcgataataatcggccgtaacttcaatgcgaagagaatatacgattctgaacagttacagataaggcctattaggcgcgaaaatagtcgtctaaaagaggagaactgctggtcgagaatgagtgggggttattctaacaaaggtagctaggtgtggttataaacgagaaggactacacccaattgatctcgataatagggcgggattgtttattgacagtagtgaggtgttctaataacagaaatttagttaaggtgcgtattcttggagtagagcacaaaacccgctaatgagcattgtatgaatccgcgacaaaagagcaaagatcacagcaacgaaagtctaattgaaatagtcctcgattatgccggtgagttgaaaaaagttgtacgttcgtttatgccgttctagataatttacacatcacattcctcacgtaactacatgatttacctactatcacttccaatcaccaactcggatttaggaatactgtaacttatttccgattatccgattgagacctaagcagaaaaacataagatgcccatccgaattgtgatgtggataccagttgtgataattcgtcggattgaactcagcctgcttaccgcttttgatcgcagtcgccgcgggtagatgtagttagcctcaccggctggatacatatctccaggaaatcgcggagtatcaatctctagagtaaatcccctgccttccgttgatcgtcttgctcacctaaatgtctgaactaggctgagaacacaaccatactccggccacgtagacgatgctgaatattacgcagctatactcaaagttaaactcttctcagtgatttatgatgtagcttagtgatctttacagatttggtatcgattgggaatccagtttaaaactgaaacgacatatagaaatatgtaccaatctaccagcgcaaaccgagtcgaagtcatattatacggtaaatcaccatcgtgtgatatattgcaatttgaactgatttttaatccctagcttaaatacttcattgatttctcgcctttaattctctgaacgttacaatttttctgcccaacggtcctcctctagaatacctcgagagccgacacaaatacagttagagaatttttggtgatttgtgcgacttattagaaccacggggtcatgaccttagcccgaataggtagtatccggatatctgaaactccaggcagtaataatacattgccggaacgacaatcggatctagtgaatgcgacatagacggtaatatgttaagcacctcatagatgattactatcaggaaatatcaatttaaagctgcgatgaaagggtcaggacccagccctttcaagtctacgtaactccactagccacattgtctaagggtgccaatcatagatcatgcatcaacaccggcgatacgcttgttcaggcattcatatcttatagttataaaatttgtttatcgtgtgcaggggtcgatttttctcactttcggcaaccaggaaaagtagtaattactatataaaatgaaggcgaatttcggattactctgcaaaaaatcattagaatacacatctaggatccggaggtatctgcctccatgaagttaactccattgtggatatgatgcgagtaacatatttaggtccgaagaaagg

SEQ ID NO: 80 - DNA, 2000 bp, synthetic construct
  misc_feature 1..2000: note = Description of Artificial Sequence: Synthetic polynucleotide
  source 1..2000: mol_type = other DNA
atcatctacctaagacagagctgaccgtatccattgtcaatagaacagcaacgattttttccatcgctggaagagtgatgcgcactagttcatttcggacaagtaacttggacgcgatacaagatacaatcgatgtcacagcctctttagtacataccatggaattatgaatcgactaaaaacgcagacgtataattcagctgatcgaatgatttcgattatataccgaagtcagtgacgagaaccttcactttgcgggataccgaactctgtcacaagaaataagtataggttagaatccagagaaaacattgaatattatgttttttcgcaccaaaataatccaacgatgttacgcttagttagtggatatcatgacttcactaaacacttggattgttatctaaagtttttatcttcctggctgcgacattgtttatttaagacgtagttaaaaaagtcgaccacggaggaggaattacatcgtcgctgatgagcccattttcgctaaatgcagtcgactacgaagagtttttcgcgtatcgtcaacataagttgatctttttagataacaaacaaaactcttcgcatcgacgtaaaacatttttcataggcgctttttacaccgaagaatctcagcttcagaattgtacgatgtcttgtcacagatatcctttaaacaaataactaatagcgttgattgtttgacatctactccttattgttatgaatgtataccatattgttatatgctattaaatcccacatattgcggttcgcactaaaatgaacatctatataacttgactgttacttgaattagttatggtccagctaatttttcattctaggcatttaatcctttatgttccatagtttccttcgacgccttgaacgatgggtgcgagtccgacggactaacatttataaacacatttgtgggtttgggtttgctacagatatctggacgcaggatgtttagagtaacatctgttgtcatttggctagcaaaatttgagttacctgatagaccttcctcattcccttaatattaaactgtctttctcgaataccgttcgcacagggtccaggaaatgtgatgttatgacggcgtgcaatggttagtccttatgcaggagtttctccgcacccatcaatgccattattttacagtcaaaaaaacataaacttgtatgacgaatgcagacctttgaacttttgttaacctacttttgtaaaaccagcgaaccctaacagttatgtaacgagatccgttaaccaaaagcggttatccgaggataagcttcctacgacgtcacatttgtcatcttccttaccggtatgaattgtatgcaggtccctattcgaaatgtggttataactgatgggtatcagcaggttatttataacgcgtactttatccttgtaggttagttgctcagtacgcccaaatcaaagaggaggccgaggtgcaggaaggacctgactgacaatcgtaactaaattatccaacaggattgttaattgacaatgtttacactgactatggcaaaaattgtctcccaaacggctgcggacagcgttctttttatcgatctgaggtagcacttgcatatggatatagcaataagaaatagggagataccagcgaagaacggagtagatgcctgtgacgtgtgccgacctgacattgattatcgagcatgcggattaaaattcaacaactattcccgtgaagagtgccagcctgtagtcaattattgtggatattatctaagttcagatcatacctctcgtcggtgaaaacagatagaggccaaagggcaaatctattgaatgattgacaatttgatcatatacgtgtctaagaattaattgtaacggatgcgaattcgttaatcttcctggggtactcttctccacgtcacgagagataacaacaacatcaggcttctgataaatagcgtaacaacgtattatcaaatgcatcctgtctgtat

SEQ ID NO: 81 - DNA, 2000 bp, synthetic construct
  misc_feature 1..2000: note = Description of Artificial Sequence: Synthetic polynucleotide
  source 1..2000: mol_type = other DNA
ttaatgacccctgccttactgcataaatctcctaattgtgtaatcactcctcactcagataacgctttacgtatggattaccaagtaagtgaaatcactatacaagagattgcctaattttgctaagttagcgttgttcgtgttttataattttattgtgagtctttcaccgaagtagaaggaagtaaactcgcagtttcttataaccacttctaggcgatgtagacgacatagaaaatggggtaaggaactcataatttttaagtcaatgatacagccttaaaagataaaaattagattaccgtttaatgagggtacgtgaccattaacagtaagaaagcctgcaagcatgggacaggtgctattgcagagctcataaacgaaatgtcgcttgggcgtcctgcaccagatacttagtggcggatgtcaatagcgaggacgaatcattggatgaatattagctagtggatacggaaaaacgtgactacgattgcggcatcgagttcttaaccctctcatggaggcatctctcgaccttacacagtgagagtgcattttgttcgccagtctactatgacacattaaggctcaaacacgctctgcttattcatttggccttggggttctagatcacactacaattgccctttgcaagaaaaacaaatgtcattgaaaaattaactgctgtcttataaacctaaactaccagatactgtaattggttttaggtttgagcatccaccaacaccaatagccaagattgttaaactctaataactgtctaatacacgtgcatattcatagtgaatcagtgcggttcattttctgaagagctccaatctgaacgatacaaggcgtcctgcgcgtggattaaaaacaacttaagcgttacgcagagcagtattccattttataatataccgtttgccgcaggaggttatattgtagaagattagttcattttgtgggggatttacaggccaatatttaccaaattttacgaggtagttgaacctagtgttacttcgtgaggctcgaacggtcttcccgctccaactgtacctttagatgggggcttctttggatgtaacgaagtaccggcttaatatgagacgtttgtacgcgaggcattcttatttaacccatacttaatcaattcaaaatttatcttggtgagtagcactggagaatttggtatccatagcggaccgatagaaagattgttataccaaaattcatgaatgacgcttagtattttctagtttgataacatggttaagactacattctatccgaattcttattaaaattgaaatgacgcattgcatgctgtgattccaaaaccatgccgacaggaggtcttcttaaaaattcagcgtgaggttactacaccttcaaaagtgcataattggtggacaactaaaggataattgggtaagatctttctacattccattaaaaaattctaacaaaccctatctcatgttaagtacttatgttgcctcttactacattgaccctacactcagatatgataaattgatgtttaacctaactatttaaaagctcaataccttcctttttacgcgcaataaaaggttaggcacttttaatgtgaaatttcagcgaaatattcgatcttgatataactaagtttacagttcctattactactcattataatagaatgtatgggctatgaataataaatggacccttagaaggataaatgcattgattcgatgctagagtaaactgatggctcagacagaatcatgcccatggggaaacataacacctaatcagcatcaactaaaagtcacatgtacgagagcagaatcaaatacaaatcaattatataacgtgaacgtagaatccggaccagggacgtttctactctgactatattaccgccagctgctatagtaatcgcgtatggagcatgtatttgctgactaatgctaaagtacaacattactgtgtaatttaaaatgctacct

SEQ ID NO: 82 - DNA, 2000 bp, synthetic construct
  misc_feature 1..2000: note = Description of Artificial Sequence: Synthetic polynucleotide
  source 1..2000: mol_type = other DNA
tgtacttgtcttcttgtttgtcacatacggaccctaaatgaccttgtctagttatccgatacaccttgcttaagtagcctcccctagggggaacttattacggaataacagttttacagtattaatcaaactcttatccacgttttcctgtgatcacaacgtattgtttcccttgatttgttgagaatctctattgagccttttatctattagagtctccgtcgcacataatcccggtgcgttgaacagatactggctagactccttacttttctatcagttgaacggaggatacgagcttcaaaataatgatttgtttgtagatgtcagagcatcgtcgtgagaggaacccggatagggggaataacaggtagcgttgcggttgcctgactaaaacccaggactcaagtttcattattaacattatttgcatgaatgacagtgtcgcagatctggtataatgaccaacgatcgtttagtagataaattccaatctaacaaacactaaccagtatctcagcccacattgcatcttgttttagcaatcctgcagatatcagaaccctcctgcagtgaattgactagtgcacgacggtaacatatctctttaatagcgcaccgtcctcaacgtagatgttacgtctggggttatattgggccggaatgtcctgggcttggactaatgaaggcaaaggctataaatgtgcttattatttacttctgcgtacttatttggagaatgtcatattaaagatgtcgcggtggtcggattaattgaataatgtgcgacttggatgcacctcaatcttcattgttttgaaaagtctggagacgtgcaattacactctatatgtctttgtattaatcgttataagctctaaaggagatagcaagctcgggcaaatggtagattaatgcttcaagaaaatacaagcctggggattcacattccgaatatacaactaatgacgctctcattctcttgcaagtatagtaatcggcccgctactctatggggagtatggcatcaggagagagtatcattgacattcgaagtttgcatactgagcaataagcgggtaatgcttcaaaacaaagtgcactcacttaatgtcggacattgtttataagtgttagcgctcaattttccgcaatcacgctcgagcactaatagttggagttcgctttagtttgataataacaaatatgactttgtcgcgagattgcctatttgcatccaggactatcgaacgcaacaaactcgtgaagaggccgcattttaactgcaggatagtaagatctaattatgaaatacatagtccagaaaatcattcgagactacttaacaaatagtttcagaggttctagactttctcaaatgtatgtagttcgtgaatatgtagttatactcaattacgactttgatttttatttaccgcctaagaaacttgattgaaataatctagaagcctcaatcctgctccatcacaaacataatatactgaaagctagagggcgttaccacagtggtacgtctagattccaaagcgtgctaggagattagtggtcgaaacgcaggttccgcgagcagtatcaccctacaaagtagctggttacagtcaacacctagcagcaatttcttcacttttgttacgatacgtccgtggcatgatcgtcgttgcctaattctacgacttaaagataccgaaaaaagcaaaatctagaaccatgatagagctacaaaatccctctacccgttcgtacgtgcttcctaatcagatcaactatgtgagcgacatagttttagctagtacttgagcgggagttttgttctcgtctctgaatatataaagtgtttaatgaagtgctatgagggccactcatctttagcatactaaatcatcagacataaaggtcacccgaaataatcaagcagaagactaacagaacatgctaagagaggtctttcaactacgcacttgatagataaccgttagctc

SEQ ID NO: 83 - DNA, 2000 bp, synthetic construct
  misc_feature 1..2000: note = Description of Artificial Sequence: Synthetic polynucleotide
  source 1..2000: mol_type = other DNA
tcacgacgagtgaggtctgagaccgtcatcaaagatcgtaacactttttaccgggctgccataacgtaagatgcatgactgcaagaaagttcacggtggtaatttcaatgagtcattgtcattccctgaaggacgtataatactatgttacgtagattattagggatccttatgcgttgaggagatatcttgccttgagtgaaagaaactcatctgtttagaaacataccaaatatgtcagacacggtcggctttgataagagtccctaactaattggctgcacattacgattcgccgaaaatatatgttgggagtagtgtacacgattttagacaaattcccgagatgatgaccgtgacatgtacaatcgcactaaaaatccccggtattagactttgaagtggttttggtatgtgatcttaagcatattcactatactagcataacaatggtggttgcttttggacgcaagttctgagtatatgactatgaagcggaatcgattaattatgtcttccaataaagcttagaagtatggttcgtgaacagcttccagtataatttagagaggccgacaatatatatagggttttatttactattggccaagaacatcctcagtcgatctaaacttcttccaaagcactaattctatcgcaaaatggtattataacaacactaatcttggagtcaactcatatacgcgcgtgtagagtcatgtaatactcagcggctaactacatgtattatgtcaagtcttccttgctatgaatactggtattcctttgtggattaaaacggtaccgtcatgtaattttgagataaagatctaggacggggaagaaaatagtaatacggtatgtatgcgttgagttgggtctggatattcagtcaactatgggtaactgaggactttgacgctgcatcccctgctggtgcgtagtcctaaaaaaaattctctgggacaatatgtcttcacaagatccttgtgagaatcccgcttccggtccggctgggccatatagactcctattactttcaaacttcgcacagaatcttaaatatgagattgtaaggaaactatcagatctgctctagacaccgacggaggagctcccggaacgttccaaagcttttttttctaagtgttgcacttggccggtcgtacacgcagagcggtagataacccaaatacagttcttctctatgtctacgcccattatgggacgcgtggagtctctgtgacgttgacggtttataggttaagtatgcttacggatgaatattaatgaatcgtcgtagttattgaagacggccgatgtagtatgcaccgtcagccgattccaaactagtatcttgctcctgagttactctgttagattcctgtcagtttatccattttagtgtagaaatatccttgaatggttgtaccatggctcctagaactagacaagataaaatgttataccgtctggtgaacatttaacctcgtacttatccggactaatggtaattgtcgaccgcctcctgaaaactcgcattggtgtcgaaaaaagcaatgagcgcgtatttttatggagataggtgcatgtattagtctgtattcttagatgctctgtcgataacatgatgtaatgcgaattgattagaacaatctgagaggctgaaattgattgcctgcccaaacacgatacggttcgatagctagctgccgatgcgcttcgatattaaacgtaggcaaagacttccattctgttggtggtaatcctatcgattccttaatgaacccacgacattggatattgatatcgtgcttagatatttgccaccatatgatgtatataattaaaatacatatgcttaaggcgatagtatttactccctgtacgcgcagttaccgttggcatgtaacaatttaatggcccaatgaagcgactacgaaccatataatttgctacaatagtactattaacatgctatgaatttatgcaaaaaaaaa

SEQ ID NO: 84 - DNA, 2000 bp, synthetic construct
  misc_feature 1..2000: note = Description of Artificial Sequence: Synthetic polynucleotide
  source 1..2000: mol_type = other DNA
gagttgattttccgcatttcatggaaatataatagggtaacgtttagttacggaacgtattcttttgaaaactctacttagtgtcgcaactaaacttctctgttttagtacagtcaggattagagactactaagaaattcctgatctgctcgctactgccacactttacgcaggaggcttgttttcgcagtaaccggtgagttaaggtccaacagggtcagatgtcccttttgtcaccacgaatcactggctcattagaaattgatagatttgttaaaacgaacctctatgtcaacaaatgcttggaacgtcattatgacagtgttttgatgtcagtttatccagaagggcgagagggtcatggcgcggtcaattagaggttcgcatattagtacttaggtattgtcagatcaccggagtttggaaaccctgcttgtgtgatacctacaacttaacttggcccaacatgagaacgttccatgcttctggtatccgtgtttaagctctcagtggagaaattcttaaaatgatattcgtaactaaaggcatgaaacaaaatgtgaggatcggttataatggacacagtcctgaccccttcgattgacctaaaatattgaaactacattcaagtagcgagaattttttaattgttcctaaagttttattattagataagtggtcgatgtgtaggaaataagagatgataagaaaaccagacgttatttaaagggaaatgtccaccagtgccccagcgttataacatgatagccaagaatttggttatacgcaaagttcgattgcgtgctcggttactggagatcaaattaatggagcttcaataatagtactaaatcatgttttcaatttcttagcacatccccactaatagtttgtctcagatattatatgatatagttgatcgaccctgttatacgcctaaaaccaattctctttcgctacccgagagtgaaaacatattcaaagttgtcagcctcgacgtttaatcttcgtaataatttgtcggtaacagattaaatacggaagacaaatattattatcttcaactgtccaaattctccgtctccatttgagacttactcatacttcagtgaccttggcactatagctgatgtttggagagaattaaaccgagatacttataataatgagagctaatgaaatggtagttcgtatatgcggttatagactgtaagaactatccaacagactctgccgcactctcagatttcatcttaggctaggttataatgtatgggacggctcggatattctattgaatttaacaatttcgtccaacaacccttggtaactgagtttcccgattacatgacgatccagcttaccgtaaccatagaacttggcaatcctctccttaaggcgcatgactagatcatcaatcgcacttcttcaatcaagttctctatctggcgcggacatactgttttacgtctcgtttcattgtaaaaacccttctgtgtaataagaacacgcgactttgatggttgcgatccctacgtaacgtgcacttaactacatatacttggtgagattgtgctccatattgaaagtcgatgttaatcaagacggagttgtgattaataaaatggcataatacacctgtgtttttcctatataatccagagaggaaaataactgttttccgaccaagtttgtactagatttatgattttccgaatatgcatctgcgtgagtgtgtacgtctgtgtgcatacgtcattcagaaagatcttccgtatgtgagaccttttggatcagttgttcatttttgtacctgcctactttagaccaggttctaaaaggctcatttaacacatgattattatagatcatataaccattactcctaatcaaatttgtgccatcgttgcaaccgaaatcgtctagcaagatgatcatcgagcaataccgaccctttatataggctcaaccctatattcagaggaaaatcacggtttgtc

SEQ ID NO: 85 - DNA, 2000 bp, synthetic construct
  misc_feature 1..2000: note = Description of Artificial Sequence: Synthetic polynucleotide
  source 1..2000: mol_type = other DNA
gtccatcattgactctgttttctcgaggaactctgcaaaccagataagagattattagcatatatgtacctagaaggacatattatcgtggacatcccgggtgtttgctatttgagatttattgattgttttttggtaaaagatctgatttacatggcattatagccgaggctcatgtttacattagcatagtaggctggactagttgcgagagattttgttacccgggatcaattgccattacatcaaatcacgtgaaacgcttttccaatacatgcatatcccagccgatacttagtacgagatgatagttgtacgacggatatataattacgtctatacgttataaattgtcacctgtcaccactttctgaattaaaagctgagggacgagccgtattaatactaagagcgtaagagcctcctagggttatataacttccgcactcagctattattattgaacctgcgtacaagtatctacttattcaagttactacgtatgaattagtaagcatcttgttttacttatgaccgcaatttcatacgttgcatgataagacaagttcaagcacaataactacggcagtaggaattgtggctcgacaagagagagctgttttcgccgttctggggatgagcatatttaaagttgtttaacacatcctttaacgataacaaaagacatacacaggatgaggtatttctgtcaagagaattggtagtttgtgttaagaagatccctgaccgtccttagatggaagaattaacgtccatagctggaggtgttgtctttattcacggaagcataagagactcgtagtacagaataagacggtctcagggtatccaccaggatcaacgccagaaagtgggcaacagatcggaagtggaattcggaacaaacttcatatgtgaaagaaaagctttgatacgacttccatgccttggtgataggtcaaatttagctattagaaactgcaatgggagatgttcgtgcatgggaagtaaatgtatcgaccataatcgctctgcgggctagagcttgcggacagttagcggttctttagacgggctgaaccctatcgagaaccgatacagcaatgtagtccattacgacatatgtgcttcctcgactttactggagaaccttaagacgcgatggattatttaactaaatttccagttatctgaactggcataatttacaacaaacctaaacattttccatagaaactcgttatgagcatttcatgcagtgcgtccactgtgatatctgtaatggtaatcggtcctcatgcgatacggctcggtagtttgtcttgcgacttaaggcaatgatgtgtggcatgctgtccagaagcagatagatcagggtcaagtattgcccgcccatttaattactaaagagaataatgcacataataatctctattgttaatgatataattattctagtgatttatatctttataaggtaagcgatttcaacaaattaaattaaacgccataaatttctagcaatttagatactgtatgggactattagggactccataattaacgtatgacatactacactaataactaaactctatttgacagttgcattgcttaaacacccttgtgtgttaaaccatacaaccttatgtctggctatatttgtacttcaggaccgggattcatgataagtgcttaggaacctagacgatgaatcaagatcaacgtcttatttataaaacgttgacacaatattaatcctacaagatctaactttaccattaaacagaacttgctaatccctaatgaccaacagacttctggcaacgagaaaaaaataatcataatttgtgcggtacactttagcattaatttctaggattcagctagctgggcctagggaacacgagctttacgtggcgtcgtccgaatcgttagagaaacattgtgagatactcgatatttttatcggtagaatcctccctcattcttacaatgta

SEQ ID NO: 86 - DNA, 2000 bp, synthetic construct
  misc_feature 1..2000: note = Description of Artificial Sequence: Synthetic polynucleotide
  source 1..2000: mol_type = other DNA
ctcaacagcattctatagccactaatcttatctcacaggcgcattgctgccataccgttagagggtttatgagtgtggtgccaaatttaatttccagctattgctgagaagtcatataagtttaagtgcctctattcatgaatctacgaagactacgccgtctgcgcactggctttgccgtcccacttaatttaacgttaatatgcaggtccgggttaattcatgaaatttatacgagggggtagattgtcgcattatacgctcacctacaaatctgcctatcagcacagccattatgactagatttaccggggaattttcatatacacaaaccacactcattttcccacttataggattgagtctcagatcacacttgtgctgcttgctgcaaatccttttatcattgttcatggttacttgtttaactaatatcattcatttaagatagggtatctttataccttgaggccaagttttttcacagaatactgaacatcgaaacctttacttcaaatagatcaggtaagattgtttttcatttaaagcgattcgctcatacagctttctgttaatagtgatatggattggaaactaaattaccgagatatatcgtcatcgtcggcaagcagctgctttatactaggatacagaagacggccgtttccagtaaaaaaaccgccgattcgatcttcgattattacctttttacttgcggcaccaaatgtagctgaattatgttatgagctatgcgtagtataccccctttgtcctagtgctaggctctatcattttatgaaatttaactcttgctccaggatacgtcggatgtacttttaacaaaatctactgagaggacaggattgaccacgtaatagtagaactgataggcgggatgataggatcatgggcagtattgctgattttagaccttggagatagctgcttaatgagctcctcgacctcacacttactgcaaggtcaagataagaaaatctcctaaagatcaaaccattccaaattcgtgtttacataaattttactattatacatcgtaatgttaagtgatttagctactgtgtgtctaggatccaggatagtcgtctaagaagccgaccaacgtgctaaataggatttgaacagcgttatagtttagtttataaggttgtctattttatcagttactgcacgacacatatactctcagagaatagggtatcacggtatacatcgctatcatattgactaacgattgttcacggcttatattttcacgagcattccaatgtggtaaccattcgcaatcatctgggctctcagttgttaatgtagaatttaaccaggttccgtattagtcgaaatcgatgctctatgacctcaaccttcctcttgtcatgatagggtgactaaagaagtttccgatacgcgacgtgaagtccgattattatccagatggtaaagtgaagcttaaaacataagagatcattctctctgatgagacataatgatatcatttcaaagttctgttaataatacaactgctagtcaacggaatcctttccatctaaaggcgaacactaactaatttgaatgagaaagataacactaaaaccgccaacctagtagttacttgagctaacacatatattacttaagtagctttatctctggtctaagtcggaggtcacaatgacttggacttcttttagtttttcgagtacaactagacaatgacctcccgacgtagcatatagaaagttagaacataggattaccgagtggtaatagcccaatcaaattatggtgcgaaaagatagtactgtactcattacttccggtatgggacaaagccgatctatttgtcggagcacgttaattttatgaccggctaccctacgtttactgagtctaaaaatttgtaaatacaaaaatttttcccgcgctaagttaaccataactctcaagttatacggggtaatggatcttaagttcccggaaaa

SEQ ID NO: 87 - DNA, 2000 bp, synthetic construct
  misc_feature 1..2000: note = Description of Artificial Sequence: Synthetic polynucleotide
  source 1..2000: mol_type = other DNA
gtaagactgattaagaaattacatagggacctggaaccggtatcagatttcaaattttggataataaaccgccaggtgttaacccatcaacatctagtattggcgtagtgagatctcttgcatttcagacatcctgggacggcaggagtttctatccattttccgcaagtgttatgctccaattgacagatatgtcgccgaggaacaccaatctggagaatatttagtcgagaggcacaactggtgttataatcttagtgttatcaagatgaccttttggagtcctttggatacatgaacccatacaaattatcagcgctctactcttctgtaacacctcggaaatacactgaaacagatgtcagagataaccatgagtggtgattgcaatcggtgaccatgttcgtagatcagtcctacgagcgtccatatggcgacgagggaactccacctttcgagcaatcatattggattgagcaaatggtcattcaaaaatatactgttcactctgccaatataaaaatagcactcgttttttctattaggacgatactaagtgggcactttatccctaaataactttcacaaacccgattatagatcccccgtatccaactggtagaaggcggctcggatctatcaagcatttgccgaattttgcgtgaaatttttccactgactgctaagcataaaccgatgaagccaatcttgaatgggttatcttgaaaatattttgctagatttcatagaaactttgattaactatatacgatatacttatgaataacgcgaattacatatatagacatgttctacgttccctgaccttgcgtcaacaaaaatcggttatgtcttaatcagaattgtattataatacatacgtagccgttttttaactactgcttataagagaatatttctatacttactacacagatgtttggactataaatagaatgacatgggggcaggggaatatgtataaatgcctgtgtgatctccaactgcgcattttgccgatgatatgtagataatactttgagtcttggacggccaacgcgcacagactacacactactatagacaatggatgatttcagacgcaataaaatgctaaaatcctaccgattgtcatatttttaagtctatacctcaccgtatattgaattcatgtcgtatccgagcgattttcgatttgccctgagaccatagataaaactcactgagctctaacgtaagattcaattcaatcaattataagagcaaaagtgtaacccgtcgaagttattaagctgaaatagtcgcaaaaactgtcaggtattgctgtccaagttagcggggcgccatgagaatgtgaatgacacggctccttgatatcacagcgtcaatgtttaggtggattagagcagagatataacgaatgctcatccgatatgacgtataaacaaatgagtaatgttaacacttttatactccggtacctcagtattccagatctgacgtccgtggacacagtcctcaattacgctgttattgtatggactacccatcgctgcttgacacgatcttgaatttatatagctacgaatgcagaggttttgcaccgcttggcactaccgagtataaggattatgtcagtcgaggcctgaagcggggactgtgaaaagcactccacacacaacagccaatgtagagccttcgtgtttgaaattctaggttttcaacatagttttttggctgctattctattaactactagctttacttgtaatcttcggctaaagtaggaatgtattaattcgctcaccgaatatcgcccatccttgaccacgatgtcccgtcaatttgtaaaaggcatctagtattcatcacggtatggtatcccttaagttgtgtatggctacaaaaaagtaatggaatctaactaattccatcatgcgcgattcatgagctcgtgtctgtatgaaagaatataccattcaatagacacaacaatgatt

SEQ ID NO: 88 - DNA, 2000 bp, synthetic construct
  misc_feature 1..2000: note = Description of Artificial Sequence: Synthetic polynucleotide
  source 1..2000: mol_type = other DNA
caagctagtctaaactaacaacagcaggagggcgagaacgttggccacaagacattaggcgttctgtttatcaagcatcgacgtctaataattttaatactaaaattcgtcactatctagttgttcaccatggatttttatgtaggcgatatcaattcagtaaggtaaccctagttctctgggctcatgtatgaaatcgggaagaaagatatgaatgaaaagaacctaactactgaagggtagtcgacgagaggcagctaataggcaacctttgtcccttcggacggactggttgctgaaattaatttacataaattaatgaaacatccccaacgccaccttacccatagggcgtctcacgctatacggtctattttaatgcctaagaatttacgatgagcctataaataccttagttgtgaacgaaacgcagcacacgacaatcgtacaacctcacttttaatgttatatacgggcgcggcttggtaaatgccgtagctctagtaacataatgcatcctcaccataccagcaaagctaaaaatcttcaaatattcgtataaaactaaccagtttaacgtgtatgaggcggtctttttaccagtttgggagcatattgcacgtactatcttctttttagcagacctgggatctgagaacttcccctgggtagtcttacgattatagttagcctaatagattatttgttcgttaggaagaattcatatatactaggttatccttcaggttgaaaattaaggacgttacagatttttcacaattataccgactaccataagtgggagcgcgaatagcatttgagtatttggatcaagcatctgctgggttacacgtattaattagacccttgccgagatctagggaaacaaaatccagacccgcagtacgtgggtggtatgacgcttcttaggataggagcgcaagtccatagacctttatattactacgtttacctgatctaaataatctgatagaaaattaaccaggagtcccattaaggtattcaaccacggaacagagtataatctggttgataaagtcgttttgatctgttaaagatttgttaaactaaacgagacttctttgggtaacatcatacaagtctgataaaggatgatgcagggactagtctaaaatgagggagtctttgggtatccaccaaataatttcaggagttaagagcacttccaacgatgcagtcctttggccttctcgtgcgacaaggcaagaaaagtttataactctacagcttgtgtaactcgaaagctgacctactatataatgttattggaaatcaaactcagggttatcttcaaacagtttgttattggctagacagctattacctttaattggtccttaatcttgcctatggacatgctccacacattaaacatacttaatggcatgcaattatagattgtcccgttcattcactatagcttcataatggttggggtagtacacgcaaagtctacttatatgggcaacgcgccggcccgtctttcctgttaagttacgggaggtcgctaattactattttactgggaatgcgcaatcaaatcttgattgagaccaacgccaggcccgaactattcttattgttccagagtctttacttgaatgcatagtatcgggatggggtgatgccggccaccggatcaccatggatatacgtcagttggcccacgtgttaattaatgtcatattgttatgggctaatacattactgtattgtttaaatacaattcgtcatgcattatcagtactgtgtaatttatataagcgttcatcattgaacgtgtattttgttggtgcgtactgagttagatattggagaaattccctaaccaaggaacaatgactggacttgttagcgatgtaagagtaatgcaaaagttaatgagactgatattggaaacagtattgtttaggctagtctagaaataaactgctcataaagaatcttgcagttaatat

SEQ ID NO: 89 - DNA, 2000 bp, synthetic construct
  misc_feature 1..2000: note = Description of Artificial Sequence: Synthetic polynucleotide
  source 1..2000: mol_type = other DNA
ttcactattaagtacacctagtcagacgtgaaagttagttcttttcacgtctcatatagtgctattttcgaccacgtcttgcaatcgtgatagacagagctgtcattaacaagatcaagttataaaattgtacgggttgtacctgcttatagttatatgttgaaattgcaaggccgcgttgtgaccggtttgacggaatctgaagggattagaggagtttatatttaatttctttcatgtagagatagaacccaataacctctcgctacatagaactaacgttttcgcagtgatttaccttgtgaagtgcacagtacacttcactgccttttactcgcatattgatacagtagccaaaagtatcattattagtgcataaccttcacctattccaacggttttacgcattctgcgtacgttcgattgaaatagaacaaatataactataattggtacccatgatgtaacattttacctcagtaatatgtcgaagataggctaagtccccagctagcgtaactagctaagccttgatgcgtattccttaatcttgtttaacgtctctgcttacgctagtttttagtagagcataagatagcaatttcaggatggaacgagttatagaacagaccactcctacagtgagtagggtcacatgtattgtccgacactgtttattcaattccaatcttttaagtgcgaatataataagaagcaccctttcaaacaattgttataatacgttttcatgacaccaacgatgtcgactatgatgtgcttctcttttggttagacatctttgcatttcgacgactccttttcattgagcaggttttagttagctaagtgtttcctacattgtagcgcattagtctaatagagagtgagcattagtcacaatatagtccaatggatctgagaagccttatgaggcgtgcttagggaacaattgcagtttaggcagaaagagttaccctttaagggtggtattcttatctcatatctatcttattggtgcaaagtttgtctttgaacgacagagtaactccattcgcagccttgctaaaagtggagagacgcaaaagtggaggcacaggtcgtttcttttagtcgtatatccagtttatgagcttcacatttaagatcaaatcccttctcgaaataaaaaggattcccactttaaataggcgattgattgtgcgcactatttattcgtaatctatacgtaaagaaactgaacgccacagcctaatacatgctagtatttcatacatgtgagccgaagacacgcacttcctttttgatgcgagaatttagggcgaccaagtctggtaacattctgtcctagttgccgagtaacatagatataagccttagcagggcgcggctataccttggtagtaagacgggtgtttgagtaatattagtagcttaattaacagcggtcaatcgccaaacggaattgtaactggaatgtcgtataatcccatttatatctcagcacataaatcaaaatggctgtgagatttaaagaggttagtaattgttcagaaatccgaaatcctcataaccaaataaaattcgcatatgcatacttgatcggcggagcgatgaaagaattacacttttagtatccaattataaacatcatttgcggcctacttttcccagtaaatcaatacgtggagaactggctcgtactctgctctacacttattgaatgagttagccaatgtagagctggatactaagctctagaagttactccagaacaattaccacgttaataacttctattattcagagtcgtaacagccctcaagtcctctcttgttcgcctgtcagcaatctcctacggacctaccctgccaggtagttgctgtctaagccactattagagttgctagatttgttaattataatgcttcgccatagtcatccacggtcagggcggtacctcgcagcttgtgtaagggatccctcgagtaactcttgatgat

SEQ ID NO: 90 - DNA, 2000 bp, synthetic construct
  misc_feature 1..2000: note = Description of Artificial Sequence: Synthetic polynucleotide
  source 1..2000: mol_type = other DNA
cgtagtattttgtgagctagatggagtactccgattcaaggtattatgaacgatagataccgtggctatatcataggattgctacactgtaggttccagaccttagcgaagcggataccttccgttcggttatctgttaaaaactttacatcttcatgataaagtgtgcctacctttgtatcactgatgtacttccctacaatagatactctttaagacctgagtacgccgaaagaatctgttcgatctagcaacgacaaaacagttatcagcatatccgtatattgtggtgtagcgtcttcgtgtactaatttagatttctgcatctgtctagttacgtgtagggcctatgacggtcccttgcttttcccgggaaatatcaattgcagttgtgaaaattgtttataggaaaacacaaatctaaataaattactccaaggatcttctcccagatgactattcttagataatgagaaagggagactcgattaagtaatattgtcgagcaccacaatctgcctatattctaacttagtaataattaattaattatgagtcaaccaaagggtcgtttagctgattcatatacatactatatttgatcaccacctacgagcagttggcataatttccttgttgactagttttgacccacgtgattcccctaaattttttgtgctctatgaccgacaaccacagtgtaatgtctcaggtaaaaatgagtacatactacttttccagattgcataagttatagacttcggtattttccaaatattattgcattgtactacaaaactaacgggtatgagtagacacaaacgatcacgggtttcacttatgaataacgttgtaacgataagtgcgcctcgcctgcaccgcatcactaacgcctttttcgaggtaataccacgttccgaagaatctatttagttcctcgaataaaacattattgataagtagtgaatcaccagcctcccaaaaataccagaagagagaaacaggtctttcaattgctggtactatttgatatcctttacacgttttctattctccagtgtaagtctcgttatgcaagtttgtcaatatcagaacaatatgatatacaacacctcgcaagctgctagcagttagatgcgatccgatgatgatcgataaaaacttatgtactggacctgctggtttagcctttaagaataagttgattcttgacatacagctcgggcgataggattgaagagtaaaagcgatgtaaaccaggtctgtgttcgatgcagagcaagttcctgcatcggatttttcggatatgcagcttagatggttactcaaatccaattccgggctgttgtctgtacaatttgggaggttgacattgccacctgggcaaatgttgtccgagaattcgcccgatgagagaagggacttggtggagtcacaagaataggcgatttcgccccaaatttaatatccaaaagaaggcgttctactaaccgtaacgttagacatattcgtacagtgaagttcgcactatgtgtgcattactcaagtatctgttgtataggataccttagtggttcagtattaaacacgattcttttatcttgtatgttgtaatagcgatcgttacttatcaacagagttaaaccatggtacaagtgcacaagtcattaagcatctagactgcactacatcgcttctatattcaccatatgacgttacaatctcccaaagtaagtatgtgacaacttctccggccagctacatccggtagaattgtgttaactaacagtgtaattatactccatcatacgatttaaccggttgaatgactaaaacttaagtagttctcgcatgggtctccgcctcactggtaatatgtgaccgctctattgaattcgagaccaggatcaattacatcctcaccgggtaaagagtagatcaggatttttaagtgagtaacctggcgatgaatacaaggttgtactgcagttttaccctga

SEQ ID NO: 91 - DNA, 2000 bp, synthetic construct
  misc_feature 1..2000: note = Description of Artificial Sequence: Synthetic polynucleotide
  source 1..2000: mol_type = other DNA
gatttaaatggtaattaaaatcgaaggttttaaaaggtgagaatttttttataaaatgcaatctgttacgcccctaatattcggtttcatgatttgcttaatattgtatcaacacaagcatattgttaaacagtctctgtactttcttgatgaccaataatgaacagatgaagtcttcatatattgaacttcaattgaatgcgtgcatgccattattcgtcatcgagaattaggaagaaaacaattgcagccttctagcgccaattgcgattagtaagcttcgccctcacgtactaaattatattagactgatcggagacattaacaagctgcttattccgtcttgaagaccgtatttcttactgttacggtgtccttaggcgtcatatatcaactaatataaaccggtactttattcataatagccgatattcagtgattgtttgccataggctactttctttcccaaatccccggtatcgctatcctatgatttctgcgtcaggggttaattacggcgacaccagcctaacccaagatcagactaggataatatttcactggcaatactcatcgattaattcaactagtatctattttttcacactccgcaaaaaagggcaaaacaaagtcgtcaagccgggaataagggttattcttgcagtcttcgtaataaaatttgaactcagttattgcgaatttactcgtataaagcttctattatcattctctgattactcaaaaacgctccatgagggtagtagcacataagtagaattgctcatagtggcttctttctctcaatccctttgatactcatttttatattacttacatgtaacgattgttgaaggccagcaaaccatataagtggacagaacagggaacaagagaaaataatacagaaagtagtaactagtcaagaaagtctagatgaatctataagttgtacctatcgaactatgatcgtagcattttcagtctacttgagggagaggctgtaaggaattttagcggccagatatatatcgctggaaccaagttatcgcatggaaacttgatcacgtacagaatgtgatgtacgcgcaaattagatctgaaatccctctgtcctcattttttaattaatacaattaatatcaaaggccttcttttctgaatgttattagacggaacacggaactgcgattcatcatcctaactacacaacacgaactgaccagatttgcgtgtaatcgtcacgtgccgttgcttactctagtaaaccccggcgcaagggcgaattgtgaaaaaatgagtcaattcgctacagtggcaaaaaacgagctcctggacgacacaacctcgtatagcaaggcgtagctcaatgcgccagatattcaggtattgtagcccatgacaacaagaaataaagctatagtaggcatcattatcgtttcgtccggcagcttttttctgacttccacctcattgcgtcttatgtcattactgcgtagggtcacctatatgagtcttcatccctgggacactgaagggagtacgccagtatttcatctatgaataaacctcgattactcctttatgagaacaatacttacactcgacggggtcttgtggtagtgatcttaagattatctaccatttgttcacccttgaaaaaagagacttacctctcgacttttttctatactgggccccgaccgctgacatgcagaatattgaggagatgcagattgatatttacaaaaattaaagcagatactcaacgcatattctatgaaaatcagggacacccagggtggtgctttaggatgatttacatgaaactttaaaaggaccgggataaactggccgccggtctttcactgccacagggatcttattcattcggatatattattgccactcaagataaattctgttagtaagtgttaaagtgtatcattattgcccattcttcagactcgagaacttcgaaggcaaatgctggacgtgtgtac

SEQ ID NO: 92 - DNA, 2000 bp, synthetic construct
  misc_feature 1..2000: note = Description of Artificial Sequence: Synthetic polynucleotide
  source 1..2000: mol_type = other DNA
agatccacggccctgaaatcgccatcgctgttcttctttgatgaataatgcaagggctgagttcatcagtgtattcgaatgctactatatttcagtattgtgagtatcacagctgtaatcttcggaaatacaaggatgtttgtcgacctcgctaacactagattattttggcccgttactatttatatttttatgacttcaaaatgcgcttcaagattgtaactctggttgatataggatgcagggaccggctcagggccgctctgcactacattaatacctcagggatctctatttcgttagagcacacgacttagtgactagaatagctttaaatgtaaaacttcatcatatattcctcctggctaagccttaatttcattcttggggctgttgccaagactgctcaagagttagtttttctttctccttgtagtacccgttctcctaagtgcaaataatctatacacacttcatattgggtataccattcttggtttattgtcacctgttatgtattttgcatcaaaataatcatcgatgtatacgttaacccaggagacaatcgaccggctaattccgggaacgtagatgtatgtaaagtaacatgtatttcaatttcttctgaagtatgagatttcagttgcacaaaaggtactcagcatgtcttatcatccatagggccgcaattatagaggatcttgagtggagggtccatacgaggccttaggaagccggcttatctcagcgaaggttatcgagatgctaaatttacggataaagatccgttactcttctttagaactaccgttccaactcgaacatagaatcggctccgaattcttgggtaccttgcagaactgaaaaatagatatctcggtatctaaaggcagaaatagttttcgctctggattggtttctaaagtgaatctcaagttctaggtaagcattcaagtccattggggaccattaggggttaatacgcactgacgtcggtctttcgattgataaatacttaacctcgttagcagtgagggtcaacaatcattaatctccagctatagagcgggttagccagattttatatcggcgtcattccttttatctttgaaatttaggccaaaaagaagggaactggttctattcgcgaattgaaccgcatttatggtaatagatctgaccacgtgctactgctcacttacaatagctagttttcggctcaaactttgtataaggctcactaggcatataacgagttaaaacttttcacatgatacgtgactagcttcgcccgacatactatatataaggtctaccgttgcgggaaaagatgaagatgatattatcaagtctttgactaataaattaacttatgcttacaaatttccaaaatagatattccagtcgtctatccttctattacagagaaaggcagacttaatccgttcattatataatttatttagatgttagtctttctggtgggtcgattgttagtctttacatagaactcctttaatgttcataagtttccatcagtagaaagtgagcttatgggttattcacctttgatattaaaagatttactactgctataatctacctagctcagctgagaggcaagaggatcacatgttattgttataatgctttgattggtaaactatagtgtcaaggcaattcgagtgtcgccaagttacgtcgattagatcgatcattaaaatctaataatgtttagagtttgttagagtaatggtgttgatcggcacataagagtcagaacgcgggagtattgatattttgccgaattgcaaatttatcaacatcggttctacgtatcgttgatgtcctaaggccttagttacgtagcttacatttaatgcgcatagggttgaagcgtgtgttaatcgctctttcaaataagtgttaggaaatatacgaagtaacgaatatcagcctaattccagcgactaaaatgaaacaagagcatccggtggtag

SEQ ID NO: 93 - DNA, 2000 bp, synthetic construct
  misc_feature 1..2000: note = Description of Artificial Sequence: Synthetic polynucleotide
  source 1..2000: mol_type = other DNA
ttgatagtgtgattaattagctggtcattatcggtatcgttgacaacagtaggatgatggcgattgtctgcagatttcgtccattaatataagtaatacttgttatgatgtccaacttagatatattggagttttattgctctatttcctgtacccttgtgacgagtaactgctccgtgatataggcaagttaagtgtgtcgcaatatggcagtaggctgaataccacacatactgtctttctaaataacactaggcgactacctttaacttcatctaaggacgttatttcacactaagcactccgtcccgagaacagggtctattgaggctactgattgcgtaaagtagttggacacgcatgggttctagatcctcatctctggtttctcaacatattgagttatactttctgttagttgttaagccgggcgatcaaagcatttctacttcagaaatggaggactgtagttatatactacattctgaagcggtaccattaatgctttccgcattgatgaatatctatatttacagtttggtgaacacaattaggagagtcggactgcgcaaacagaatatttagttacttatagttaatatacacctatacacggtagaaggtcagttcatatagacttctgggtgtgtacttcatcagaagtctcctgtctgtttagccaatcgccaccttctcagtcccgtgggagtaccactcgaatagatcgttgttttcgttgttgataaacggaccccgtcttattttcgttaccatttaatacgatatcatataattgaaatattaggaaacggcatttcaaatacgaacgatttgaacttcacctaccttttgacatttatattacaattttatagggcaaaacgtaatgcacctaaatttactgcacttcagatctaccaattgatttgtcacaccagctatttaacgaacaatatgactaaatattagctggtatgcaatctgaaaagtcaacatggtatttctgcttacaccggtagggttaatggaagttctgcgcccattcgaattttagaactgaacaataattcatgaaaatttacgttagcagtacctttttgtcttactagttgttgcagaaatttaaacattacttggtagcctgctgtgtatataaaagagcgatctccgataagttgttaatctgttgctacctaagcgcttactgtgtgccttggctcgcgtatatgcccaggtcaacatttatttgtcgctcgactcgaaataatctatatcataagatgggaacgagtatgctccatgagggagccggactaggcattcaattttgtttgagtctttagtaaccatacctattcatgcgtagttaacttcgtagtaaagcagcgtttatacataaacaccaaaaaatgtcctaggggcataccaagaatctaagaaacagcgcagtagttcgttcggtttggcaaccatacgaaagtatcattgcacacgacgcatacagcatcctaggagtttactatgtcttcgtttttttgtaggccccacacacattaaattcgatttattacactcagagtacctgtccgccaattcacgtgagtaccttcgcgcagcagataatacattgctatgcgttcagaccattgtaagaaaacagatcatgactctagaaaaagtggccttagatcaataaatgttaaatccggttctctctaacctcgccgtacacagttaaaatcaacgcgcatacataaacattgatcttatgggggctcacatagtgagacaatagtagtacccagtgttatacctaatctaatatataggctaaaaggtagattaattgtctgatcatagatctcaaccgatcatggatagctgggaatacgttataaaggtaggtctacgacccgcgaaatctcgaggaaccacaacagaaaccattgtctgtacgagcgacagcgtatgtactccgtggctggtctacctcggtaatg

SEQ ID NO: 94 - DNA, 2000 bp, synthetic construct
  misc_feature 1..2000: note = Description of Artificial Sequence: Synthetic polynucleotide
  source 1..2000: mol_type = other DNA
gggtagttttttctccaaggatccccttaactagggtgaagattgggattaaacctaagataaagatataacggtcactggcgacaagcttacaaatttgcgctttacaacagaccaaggcgaaagtaatcttggccctactaaaccaagggaaatcagtagtagtgttctccaaataggcaaggctaatatctatactgtccctgcatgatgtgttaagccataggcgtgtaatgttattccttttcctaaccagcttttaatgtatccttgtgtaggaagaactgcgaagttatgttactccgaagccaaccaacatgtgtcctcttggcaccatgattcgaaggtgatattataagttattcgaccgtgaagattacatattactggatggtgtataaatagaccatacgttcattgaagcgtgactgaagccgacaacggcttacgtaatgattcaaaatcggtaataaggataacggttatatatagtagaattcgagatggaaaaaccaacttgctaatgacaatattaagggtatatcacactgtggtttgtaaagtagtcacctattcgtgatgccgtgtacttcaacttatagtaaaaagtattgttttctaaccagcggtaacctgttgcaaaaaaccacgtttaaccgattgatagcttgtggtaaagtggcatagagtatacttcctccatctgtagtacttaataggtgttccagttgcagtataaacctttcttcgagtatcatcactaagaccattagacataggatatatacaataagagctggaacttgaatcttctaatgacagactttactaattatagttcaagcgcagtttaactataaatacaattgtcaattcatcatatggtaggcaagattcctttagcctggcgtacagtggcccggaggccttgaccaaaacatggttctgttatatcacgagatggattgactatgctcgtgaatctggagaggcactaacttggtaacgcccgtactctaccgcagcgggacaggtgatagactgtctatgtaaatcgtcatcaatctatatttcaatacaactataaatccagacaagtatccttgagataatagttaatctatcctaactaataagaagaaaagagacgatacggtagtagattaagctttcgcggaaacaagaggaatctacagaaaacaccctaaataagctattccatgccgcctttgctatgaacgaagtacggaagcatgatgcttatcaacgtcaggaacctagctcaaatcaaggtcttaccagtgacgataacatgggtgcggatggttatttgtggagaggcgtaatacaatgtacttgttttcaggatatcaatttaatttcacttagaatacgagacggccgacaactttaacgaatacatttgcatcccacattaatacctgagtgccgctcatatcgtcctagcacaatttttaacagaagttttggtggtgagtagaacaacaacatgtagtcatcttaagcgtatgaaatctggctctcaaattcatgtttaatagtgtttaatcttttatgtataaatcgtttttatggtttagacgaagcactcaaaaatatagactgatgcctatgacctgtgctatctttattttccagggcaaagatgatctttccgagtccatatcttgaatgacttcccgcctgaaccaatacctggtcggaaggaggactcattaataaacatgcataaatggcagatctgaactggacggctgacttatctcacaatgtgttctaaagtccacaccgtttctgtaccaatgaaaggacgaattatacatgcattggtttggttaaaaccaatacttggtaacgatctggaccgggcggttagaatgatgaattaatgcgccgtatgtggaatgaagtcctgttaaaatgcaaaaggtggctcttcgagagttgttgggttgaatgagagaaacgccaccttcaca

SEQ ID NO: 95 - DNA, 2000 bp, synthetic construct
  misc_feature 1..2000: note = Description of Artificial Sequence: Synthetic polynucleotide
  source 1..2000: mol_type = other DNA
tagtatctagtttcaggtgtgcacagaatagttatcctcctttgtctgtggctatttggagaacgtattagaggaagcatatggcaaaatggcctgtacacgatagatggtatcatgtttggaggacgctaggcatttcgccctaaacaccgcaacgatacctaaagagctcgtcaatgggcttgccgattaaatacgcaagttttagtcagtccagaccacatttaccggtaattatgcacagacaagatattatgctggtttatagcccatatttgtctccccctaaagtgagctctgatatttggttaggtcgagtagtacagtttgctatctatggatacgatgtaattgtgcttgagatacgtgcatcacgaacattgctaagcggattcgcaatgttcgtgatgcatggagtagtctaagcaatccaacaagcgcctgaatataattttgtcacaagtaaaccttcatattgtctaacatacagagctgttttaccccctcatgatctaaatctttcgcttcttcccaaactgcacgccctattcgcctgttagcgcattcaaccctaatacagctgttgtggggatactctgattgaaacaaagttctctatggaagcttcatcattaggccatacgaaatagaatcccctgttgtccaggtgcttctcgactgcgttgcggttcttattttggctttgctaataggaacttctctcttcgagctcggtcgaacgccagttcgtcaactataccgccttctttttgcgcaaggtcatcgaaactgaggtccatcctgggacaagagatcagttaagcctacacttgtgtgagactccgcagaaaatcgggaccaaagcgttagggcttcccaattatgaggatctatggtgtcattgaaattgataatccttatagggccatttttatccctgacctgaattctatttggtgaataaagtattggtcgcctttcgagggatactactatgttatggacctaatggatgaccatctggaacattagcaacagcaactctaatcttattttatcatcttcagtgtaatatatcgtacattttaggctttcctttatgttaaattgttattatgaaagaggtgtattataagctagttaagcgcgttaaaacacaagtggtctgctgtcattcatataccaaagaaggtcttgatggacaatgtcttcacaagaccatgcatagattctaaatcgatatgacacctaacaaatgcgggctaatattcgatttctgactcccacactgtgagcacgtttattgcggagacttttaagcgagatactcttactccccattgccatatatgtaaaatggacttccaattctgcatatttcagtacatccggactgcgttataagcattgtcgtggatgcatcaccatcccatagttccacttcttttttttagttcagatccaaactacactatagggtgacttattgtcgatcaaaattattatatgtaagtaatagatcatacatcaacaccgaggtctttgtccaatagaaatagtatgtcctggagttttatcaaatacctgccatgtgcaagttcacagaataggacgcttctacagaattcataaaatcccacatccttagcgtaagttgtcagatgaattaattatatttttgatacggccccagttattctcgaagtccactcttaaaaaaagttattgtacgaacttgcataaatcgataacctgttaccaacatgccccggcataaatcaacaacgtggttcggatacgacaatatcaatcaatccgaaattcaaaatagaatattcaacttgacttaatcgcagttcattcgtgaatagacacatattagctctcgcgcgctttcttatcttcacagcttcttctcgatacctgaataagtacgggaccatttatgttcataagcattcagtgaaactgcagtctaaatactattggcatatacttat

SEQ ID NO: 96 - DNA, 2000 bp, synthetic construct
  misc_feature 1..2000: note = Description of Artificial Sequence: Synthetic polynucleotide
  source 1..2000: mol_type = other DNA
gatatgccatctatcgaggcctgttagcttaggacattacatgacagtgagacctagatatatagttgcatgagtagatgtaaccgaaggtactcagggacagaactgacggattgacgtttttcagtatcgtaaaagtttgagatccaacaatgaaagcttgatgcgccagatgatggaaatgcgcaaactgtcgtgtgataacacgggaattggtgctaagctggaatggtctaattcaagttccaatccatatccatctatgtgcgaggaatttgtaacggtaattatattgccttacaattattatcaaccaacacacttgaacgatgtaattgggggtatataccaataatagtactgccaactactgttttttgcaagaattaatcgtagtccgaattaaaagaaaagacggtgtacgcaacccaagtaattaaacgaataatcatacggtcgatatgctcattcgataaaacgcgagatctttaagttctctcaccggggtaatgcataattgccttaattggaaattgctttaggtgagagtcagtaaaccattggtgagatgtggttatactgcacctcacgcaaattaatattctaactttaacctgaattatgggttcccctcatcgggaagtatatctagtgccaacctatcacagttgcgcacatatgtttagaaatggttagtcggtcaggggaactcacgtaagcggtagtagtagaatttaatttatggtctcctaaagcatcgacatagtacactgcgaccattctaacacatactaaactttgaacttactgatatcttttatgtttgacttccttgctacgcaagtccaggcccagacagctgagttgtccttacacgagctatttgctgatcatatggtttaatcggcacgcgaattgcaagtttgatttaaggtgagcgcatacttgaatacagccagggagctccctactcagcgatcgtcttcagagatttcacgaaaatataagcattcccatcagaaattctaattaaaccttaccggaggtggggattactcgcagagttaaataatgagcccacattatgcgtttgcttctggagattatgggtggtttttcccgtaccgcctaatatagtatgcttcgactcagcaacttcactctaaaccctagagagcctctgtatgtacgcgcgtggatgaaatcaagaatggttggagtcaatgactggggcacaagtgtaatctggttcgattaatacatggcactaggtgctacgaggacgagtgaatgcaatatatgagtccttgctaataagcatcgaagatactctccggtactccttcatattcgactaatcggtgcactcaactttaggggggctccttattataaaatacatatagggtttgtttaaatgatttgttctattaatacgggcaaaattaatgcaatgttcacctaggcacgttggtactcgccgccaaacattggcattaatggggatacttagaaacaacataacatgaaaaatatctaggaacgccaacatatacgccgtgaccgtctgtcttaatagactctttttgtttaaagggtactgagtgattaactaatgctttccaatcctttccgttagaaggctattactacaagtgtttcccacgtgccgttaaaaatagaattatctttgtgggtttacgagcgcgtactgaaaacaggtttcttggatgggataatattatagatagcaataaagtaaactggaaaacagtattggatagcatgtgatggaccttgacccccttgtggcataagataatctcagcgtttcgttacacttacattcactgttaatgtctataggcaagttactatttggagtatttcaaagtgaacggaagaaatagaagtgctaacaaactccgtcatagtaggatcatatctccagagcgacctcatacatgctaaaaacctagtagacttcgtactatggattt

SEQ ID NO: 97 - DNA, 2000 bp, synthetic construct
  misc_feature 1..2000: note = Description of Artificial Sequence: Synthetic polynucleotide
  source 1..2000: mol_type = other DNA
aagacactttaccacataagtaaaccgttgacattatcgtggcggagagatactgcttgtactgggacactcagtattttgtggaatattgtacctagcgcctcgttccgtgaaagtgtggcatggattttcataattttatgctgtcctcattgcctacaattaatccagtaagcactagagaaatatctgctcctatgctgagattagccttatgaggtctttatatctttctgtaaaggccattgttcttttgatcctggagtctctgaattttgatttgtccctcaaagccttatgtgtacccggtcccggagcatgaagacgtatatcttgaagtaatccgaaagtatttaggtgtcgttgtccagtagtaatcccggttatgggttataattaagtgttaacatccgagcttggtctgtataatagtgtgtttgaatagtaaatatcaggactctacagggacctattctacttcgggttgtgtatcttccttggaataacttttgctacgcaaaaaagctataacaaggtctggagacggatgtgatttagtagggcaaatagatttaggtcttcgatagtacagaatactatgctacaaccaatctcttcatggctttatcaatacaatgttcttccttaactcagacgggagcaattatagttagctgaaggttgcctcacaatatgtgtcagagctagcgaaaagctcctaccaatatacatcagataaggagttcatacatctgtggccgatcaagcaagcaaggccgtccggttcacgacctgggtagtctgagtttggaggagaagccatcgcctctcgcattctactagagaaagatttcacacttactgacagagctacactggtacgacgaatctacaaaactaagcaaagtcctagggtgagcaatgcatggtaactagtacgattgatcagtgcgtggtatactatccggatagtccagacgtcaagacctaatcatcgtacgtaattaaataataatgcattcaactcttcggatacgatatatacttatatgcattaactatactttctcatgcattgtatctaacaaaatctgtacggcagaattaattactaaagtcttaatgattcgaatattaatatcaattttattacgaaacaaccaaactgacaacgtagagaggcaactacccagagtcgccaagaatactgtttacgaattgtagaaaagatgtaagaatgttcggatgtcggattacttaattgcgaacgtttgtcaagtcgttgcaggataccctcatctcctcttcctagtgaattatctgaaagtactattatacaatctaaatcggatacattcgtttgtaacaccacatggttggctcagctgaccatttacgcgcgatattctgtgctatccgaaggcgtaaaaggaattcaagtcagtctcctcttcgttatgtagaaagggaggactcctccgccgtatattcagctggctttaactaggaacatagttgcagttcaaacagtagaaaatcctggaagacatttcttgatagtctatctcagaaaaaggggggtgacgttcatgtttactaagacttgaaatgtggctccgtatctgcacaaccaggtttgggcggatgccggccgccatgtaacactgaacctcgcaagaaatgcacaattgaacaaatgaatactcacatcttatcgcttaatgttaaattcaaggcgagactggctcgaattattggagcctatgaagatgtatattaatgccaaggcaccgcacatagtaaagactatactaaccaagtgtgatattcaatcgatcgttgtggggaatcaggtacagttagtggcgaacagctttgacatccgtttaactttggcagcaccacaaaccctttgcgtacgtttttgtgttataaccaagttatgttgcaacctactttgacctcttatttctttgccgcaagactgaatgtcgtattat

SEQ ID NO: 98 - DNA, 2000 bp, synthetic construct
  misc_feature 1..2000: note = Description of Artificial Sequence: Synthetic polynucleotide
  source 1..2000: mol_type = other DNA
gagcaacctacggatatactatcgattctggacatggtaagtgtgttgcgtggttaataaaaagatttcgtggtcgggggtagatatacctgtaaggtttccaacagaccgctttgtagaaagagacttagtccctttgcaaaatgaggggaccgactaagaaagcgttgaattcaggtaatactttttgacgttaccatagttgttgcagtcccggagttaaacagagacacatcgtggcggagtccgtagtatcgcatgcgtggatttattgttgtaatcagatgttcaatatggcgtcaatatacaaataaacaggtcagatggagttagccttacttaaaaaacgaaaacaatgtatgccctaagcaaaaaaactagataaggacgatcaccacagttttaagagatctatatgcccctttgacatccttattctgacaatgggcagatccaactacaagatgtcgtaccgctaacacttgactaactaacgtcaagtaaaaagttcgttagtcatattatcaagtatggacttattcatcgacaggttgtaattagccctcccctagattagctgggctgaacccctattcctacgctcccttgtcacatgtattctctacctcaataggccggaaactcgcaagcccaagtatagcgtacggattaaattcgcgcaatcgctcttgaccatgttaaatgcttgcgcgtaacatcgaaaaggaggcaagacatttcagaagtaacatatcagttgacggcttacggtgctgaggtttaaaatccgactgattgctatcctatcgctgaggaatgactaaccttgcaaatccaagtctagaactgtcctagttctgtaccatgcccagcgttcggatgtcagtacgtgtatgcagcatttaggaggtgatgtctcccagtcggtcaataagctttgcttacctcacggataactaagttcatctccagtgtacgaagattctctagcactaactattcattgtaactaattggtatccgactttaagccatagtgtggcatgacgtaagttatgtcagttctttggaactttttgcgcagctgtgttgacgaaacacaggttgcaggttggtctaggtaagggatgcactcactgcgatgtgatcctttaatggccatttaaatctatctcgagtatagcgtgtatacttactatgaagcaaattagtatacatataacaatgaatatacacatagtgggaggttgccattcatccatgtaggcatgtaatatggcacctcctctttggatacagaggcccatgcctccgaatcacatatttacttaaacagttaacggaattcaggtatcccgtttcattattcgaaacgtctctggggttaccttacttacgttatctgcatgagaatagagtccatcggcgtttctaacaatcaatcatgcttgcaattcagcgagtgtagaggaattgtaagaacgccggatgctccctttaccttatccgcacaggcccctacgattgaactattgaaagttttattacaaatctcatatatgggggagcagttaaagttctgcataagaaggacctaggataatgccataaaaggttgatatggaaatactattggaataagaaagtatatggtgtctataatggatatatcagtaaacgaaggcatttcttacactttgatttcattaactgtaatctctatttgtgttggcgaatccggtaaacagaggtttataactggtttaccttagtcgagtgtcttagatatacatgtcgattcagatcaatcctactcatcccaaacgcacatgtcacgatacgtactttatacagtaagaggcacaatgtgggtgccctctctcgtccgacttattgcggacggagaaatagttagtacggactgtcacaagtctgtaaccactaaagatcgggcagctcagacattattgaaggtaggccaaagtatcattaatgctttg

SEQ ID NO: 99 - DNA, 2000 bp, synthetic construct
  misc_feature 1..2000: note = Description of Artificial Sequence: Synthetic polynucleotide
  source 1..2000: mol_type = other DNA
attaataaatgtctaacggtctagaaatgcacctaatttgctactgctgaactcctgattactcctcctcgtttatacttgttcattaagaattttttccgtctagattaagtacacggtaatacacacgattaaatacaccgccacagatcttcgctatcaatattacattttgttcactcattacgataagcgtggcttggctgagttctagacttatcgtgttaacgtcaatgaaaacttatggatttgaagctacgatgctaatctaactttaccttaagcaagaaagaccttcgttaataggacccttaaagcctgtgatgtcggttaaacggttctagtttgatagtgacgttagggactcggtatacatcttagccgaactgtctaaattactttagagaaacttttccctgggggaggcacgttccgtttatggacctcatttgagactcaatatgtacaactaatagtgtgattagatcctgattcccatacgtatcggctcgcccttaatcaatacagatccgtgctatgtccatactgcgattccaaaggttgtctaacaagacaaacttgagagaggcttcacaaagcaacccagcacccttgtcctcttttttaggggtacgctgacatctggatgcattaagaaatacgtatctagaaggatcgcgataagtcgcacaagtttaccaccttatattctgcaggctgctattggaggtaatacgtgctcgcacacgcccaagtgaggcattcttacaagacttaccttacagcctattaataacgtcgaattttgcgcagcaaccaattccagggcaaactataagccttattgaggttaatagggcgcaatatatttacgatagaaggtaaatctataatactgtcacttgtcaatgatgatggtctaactaattgattcccatgcaagtggcgaaccaggcttactttagtttaatagcgatcaagtatactaagcacacactgaatgtatcacataagatacgtaaaataaatcaactcattaaatcaaagacagattcacaaatgtttcgtgttttaacagatctgaatataaactctgctgatgtgatcgtaggacgtaagaaggtatagttgaagaatagcgtgaatatctgatctctgttagcaaatacatcacgattatcaccaggtttaccacaacaataagattgtgactgacactactttctatatgaatgtattctcatgaggatgcgtaagacgtataggatcatactgaattataactccatattagggtctatatcacatacatctccaagttaaaaagtctattggcgattccacacaactcgcgctagtagtacattttaccggtaccggtacagtctaagttattgatctaggttcaacttctaaaatactgaagtctcaggtatatagaatttatactactcgcgggacgtaaagcccctctgtggttagcgtcgcagcgtcgagtaaattccttatagagcctaaaccttgataatttcgacgtaccgttataacgcaattaatagacttctcattttcctgccgagtcgggtctggtatagtctaggacgggggtagatatgatcgtcgtcttctctaatctaatttaatctataaccacagcgtacaagtaaggtatgtaagatacagagataaattagagatttgtgttactccgcatgttgaactaaacccaaaggttcacgccgtatgcctttcaagttcctccgctcaaaaggctccgggtgtcccctacccgatatggcggaaatcgttaattctcataacgaccaaccttaccttggacacacctaagcactaagtcggtaaatggagtacacaatgtgggagttgtgtttaacataatgaggctcgttcagactatgttcgaggcgtataacgatttgtgacagattcctcatcaactcgggtcagatttatagcaatggtaaattccctatatccta

SEQ ID NO: 100 - DNA, 2000 bp, synthetic construct
  misc_feature 1..2000: note = Description of Artificial Sequence: Synthetic polynucleotide
  source 1..2000: mol_type = other DNA
tatggtgtggcacatatgaataaaacaaggagaagcagccgacaatacttagaacgtgtcagaacaatcaagatgtctgaaacgttcaacaatcgagttattccgggctaatttattcccatccttatatacagagccgcacaataccaagtaacgtgctttgggccacgaactcactctagtcttccggaccctccggtactactcggtatggtggatattcatgagaatggttttagtcttaaaaaaatgtgaacaagaaaacatttacgtccaagaaagcggtattttgtttgggtctaggaaacaatcagtcgtggacctgggcgagatcggctgttttcgaccgattttatgctaagcagaaggaagtgaccgaggttgtgtttagatccagtaaaagtcgtcatacccgaggagatttctgtggtgcctagtgactagcgatcccgtgcagcagttcaaatgcgctggatagttcgctcctgcaccactagttcacaccagaagtatgtcttttaagagactgtctaagaaatatagtctctaaacgtgactatcgttcactccctgtacaaatctaggactaacgggtatagattaaacgtattagaatttcggagcattagaattttgttgttctaagttaggatgatttcaagtgtccatgtaaattgaggtcaatataggacgatctacatccgagataggccaagtacgattctgtgttacattttgcgttcgcacaagctaggacgagggtatgagcattttgtgctaaccgaatgagatgcagcttattgtatccttacccgcaacatagggcatgaaggcgtggttcgagaatcgcgcgagataaatacatgtttcgatttatgtcaaccactgcaatggtttataaatgttattcaagcatcgattcaataacctctggatgtagtaatatctgcgggtgtgtaagtgcgatatcctaagtcgggagatttaacaataccttgggatgctccggacaattttcgacgtacgcaattatgaacatgcattgattgactaaacttaagaaacataatcagtgtatagtattgtaacaatggattctgagtgtctaatgttttctcgctccatgttataacacataattatacttataataccatcccatctttaagtacaaaaccttgttgcgctgctttatggagactattgagcccaacgggttgagtggttattactatttgaagtaaaagcagtatctactcagattcctagaggtaaatatgaacttgttttctatctggttatctatttttagttttatggatatggacgaagttaaaagttatagacctgacattcttctcccataggtatagaagtggagttaaacaagttcttagtgggggaaatgacgtacagactactatcttgatgatagcttttcgatcaaacaagagtttcaaccgctgtaaaggtttatatgcgatgtagtgtggtacgataacgtactttgccgatcattcactgattccattaggtacgacactctcagttacaaagcggtactaacctagcaaaaagtgaatatcgccctacaaactattactggagtgcggtggcagctttggcgaaaattggccgaactctttgctgtttatatggtaactattctcactatgctactgattggaaaaagatatttgccaactaatagtcgtaatgttagtattgatagggataataggcatttaaagttccctgaaacatacggtaaataagatctcttttaacaacaccaggggtggctcactggggtagcaaatacttaacgatccctttttcatcaagtgagttatctgctttggattcttacaactagatgttataaagaaagaagctgcgcagtttgcatgactaaaatttatatgaagtagtagttattagtactatctcttagtaggctagaatgtaaacctgcagacatcatggaatgcacatacccg

SEQ ID NO: 101 - DNA, 2000 bp, synthetic construct; identical to SEQ ID NO: 1
  misc_feature 1..2000: note = Description of Artificial Sequence: Synthetic polynucleotide
  source 1..2000: mol_type = other DNA

SEQ ID NO: 102 - DNA, 2000 bp, synthetic construct; identical to SEQ ID NO: 11
  misc_feature 1..2000: note = Description of Artificial Sequence: Synthetic polynucleotide
  source 1..2000: mol_type = other DNA

SEQ ID NO: 103 - DNA, 2534 bp, synthetic construct
  misc_feature 1..2534: note = Description of Artificial Sequence: Synthetic polynucleotide
  source 1..2534: mol_type = other DNA
ttatccccttcctatgacatgaacttaaccatagaaaagaaggggaaagaaaacatcaagcgtcccatagactcaccctgaagttctcaggatccacgtgcagcttgtcacagtgcagctcactcagtgtggcaaaggtgcccttgaggttgtccaggtgagccaggccatcactaaaggcaccgagcactttcttgccatgagccttcaccttagggttgcccataacagcatcaggagtggacagatccccaaaggactcaaagaacctctgggtccaagggtagaccaccagcagcctaagggtgggaaaatagaccaataggcagagagagtcagtgcctatcagaaacccaagagtcttctctgtctccacatgcccagtttctattggtctccttaaacctgtcttgtaaccttgataccaacctgcccagggcctcaccaccaacttcatccacgttcaccttgccccacagggcagtaacggcagacttctcaagcttccatagagcccaccgcatccccagcatgcctgctattgtcttcccaatcctcccccttgctgtcctgccccaccccaccccccagaatagaatgacacctactcagacaatgcgatgcaatttcctcattttattaggaaaggacagtgggagtggcaccttccagggtcaaggaaggcacgggggaggggcaaacaacagatggctggcaactagaaggcacagtcgaggctgatcagcgggtttaaacgggccctctagactcgacgcggccgctttacttgtacagctcgtccatgccgagagtgatcccggcggcggtcacgaactccagcaggaccatgtgatcgcgcttctcgttggggtctttgctcagggcggactgggtgctcaggtagtggttgtcgggcagcagcacggggccgtcgccgatgggggtgttctgctggtagtggtcggcgagctgcacgctgccgtcctcgatgttgtggcggatcttgaagttcaccttgatgccgttcttctgcttgtcggccatgatatagacgttgtggctgttgtagttgtactccagcttgtgccccaggatgttgccgtcctccttgaagtcgatgcccttcagctcgatgcggttcaccagggtgtcgccctcgaacttcacctcggcgcgggtcttgtagttgccgtcgtccttgaagaagatggtgcgctcctggacgtagccttcgggcatggcggacttgaagaagtcgtgctgcttcatgtggtcggggtagcggctgaagcactgcacgccgtaggtcagggtggtcacgagggtgggccagggcacgggcagcttgccggtggtgcagatgaacttcagggtcagcttgccgtaggtggcatcgccctcgccctcgccggacacgctgaacttgtggccgtttacgtcgccgtccagctcgaccaggatgggcaccaccccggtgaacagctcctcgcccttgctcaccatggtggcgaccggtggggagagaggtcggtgattcggtcaacgagggagccgactgccgacgtgcgctccggaggcttgcagaatgcggaacaccgcgcgggcaggaacagggcccacactaccgccccacaccccgcctcccgcaccgccccttcccggccgctgctctcggcgcgccctgctgagcagccgctattggccacagcccatcgcggtcggcgcgctgccattgctccctggcgctgtccgtctgcgagggtactagtgagacgtgcggcttccgtttgtcacgtccggcacgccgcgaaccgcaaggaaccttcccgacttaggggcggagcaggaagcgtcgccggggggcccacaagggtagcggcgaagatccgggtgacgctgcgaacggacgtgaagaatgtgcgagacccagggtcggcgccgctgcgtttcccggaaccacgcccagagcagccgcgtccctgcgcaaacccagggctgccttggaaaaggcgcaaccccaaccccgtggaagctctcaggagtcagatgcaccatggtgtctgtttgaggttgctagtgaacacagttgtgtcagaagcaaatgtaagcaatagatggctctgccctgacttttatgcccagccctggctcctgccctccctgctcctgggagtagattggccaaccctagggtgtggctccacagggtgaggtctaagtgatgacagccgtacctgtccttggctcttctggcactggcttaggagttggacttcaaaccctcagccctccctctaagatatatctcttggccccataccatcagtacaaattgctactaaaaacatcctcctttgcaagtgtatttacgtaatatttggaatcacagcttggtaagcatattgaagatcgttttcccaattttcttattacacaaataagaagttgatgcactaaaagtggaagagttttgtctaccataattcagctttgggatatgtagatggatctcttcctgcgtctccagaatatgc

SEQ ID NO: 104 - DNA, 2383 bp, synthetic construct
  misc_feature 1..2383: note = Description of Artificial Sequence: Synthetic polynucleotide
  source 1..2383: mol_type = other DNA
gtccaagggtagaccaccagcagcctaagggtgggaaaatagaccaataggcagagagagtcagtgcctatcagaaacccaagagtcttctctgtctccacatgcccagtttctattggtctccttaaacctgtcttgtaaccttgataccaacctgcccagggcctcaccaccaacttcatccacgttcaccttgccccacagggcagtaacggcagacttctctactcttaattcattacatattgtgcggtcgaattcagggagccgataatgcggttacaataattcctatacttaaatatacaaagatttaaaatttcaaaaaatggttaccagcatcgttagtgcgtatacatcaagaggcacgtgccccggagacagcaagtaagctctttaaacggtctaagtgatgacagccgtaagcttccatagagcccaccgcatccccagcatgcctgctattgtcttcccaatcctcccccttgctgtcctgccccaccccaccccccagaatagaatgacacctactcagacaatgcgatgcaatttcctcattttattaggaaaggacagtgggagtggcaccttccagggtcaaggaaggcacgggggaggggcaaacaacagatggctggcaactagaaggcacagtcgaggctgatcagcgggtttaaacgggccctctagactcgacgcggccgctttacttgtacagctcgtccatgccgagagtgatcccggcggcggtcacgaactccagcaggaccatgtgatcgcgcttctcgttggggtctttgctcagggcggactgggtgctcaggtagtggttgtcgggcagcagcacggggccgtcgccgatgggggtgttctgctggtagtggtcggcgagctgcacgctgccgtcctcgatgttgtggcggatcttgaagttcaccttgatgccgttcttctgcttgtcggccatgatatagacgttgtggctgttgtagttgtactccagcttgtgccccaggatgttgccgtcctccttgaagtcgatgcccttcagctcgatgcggttcaccagggtgtcgccctcgaacttcacctcggcgcgggtcttgtagttgccgtcgtccttgaagaagatggtgcgctcctggacgtagccttcgggcatggcggacttgaagaagtcgtgctgcttcatgtggtcggggtagcggctgaagcactgcacgccgtaggtcagggtggtcacgagggtgggccagggcacgggcagcttgccggtggtgcagatgaacttcagggtcagcttgccgtaggtggcatcgccctcgccctcgccggacacgctgaacttgtggccgtttacgtcgccgtccagctcgaccaggatgggcaccaccccggtgaacagctcctcgcccttgctcaccatggtggcgaccggtggggagagaggtcggtgattcggtcaacgagggagccgactgccgacgtgcgctccggaggcttgcagaatgcggaacaccgcgcgggcaggaacagggcccacactaccgccccacaccccgcctcccgcaccgccccttcccggccgctgctctcggcgcgccctgctgagcagccgctattggccacagcccatcgcggtcggcgcgctgccattgctccctggcgctgtccgtctgcgagggtactagtgagacgtgcggcttccgtttgtcacgtccggcacgccgcgaaccgcaaggaaccttcccgacttaggggcggagcaggaagcgtcgccggggggcccacaagggtagcggcgaagatccgggtgacgctgcgaacggacgtgaagaatgtgcgagacccagggtcggcgccgctgcgtttcccggaaccacgcccagagcagccgcgtccctgcgcaaacccagggctgccttggaaaaggcgcaaccccaaccccgtggaagctccaaaggactcaaagaacctctggatgctttgacatacgatttttaataaaacatgagcatttgaataaaaacgacttcctcatactgtaaacatcacgcatgcacattagacaataatccagtaacgaaacggcttcagtcgtaatcgcccatatagttggctacagaatgttggatagagaacttaagtacgctaaggcggcgtattttcttaatatttaggggtattgccgcagtcattacagatactcaggagtcagatgcaccatggtgtctgtttgaggttgctagtgaacacagttgtgtcagaagcaaatgtaagcaatagatggctctgccctgacttttatgcccagccctggctcctgccctccctgctcctgggagtagattggccaaccctagggtgtggctccacagggtga

SEQ ID NO: 105 - DNA, 2673 bp, synthetic construct
  misc_feature 1..2673: note = Description of Artificial Sequence: Synthetic polynucleotide
  source 1..2673: mol_type = other DNA
tactcttaattcattacatattgtgcggtcgaattcagggagccgataatgcggttacaataattcctatacttaaatatacaaagatttaaaatttcaaaaaatggttaccagcatcgttagtgcgtatacatcaagaggcacgtgccccggagacagcaagtaagctctttaaacatgctttgacatacgatttttaataaaacatgagcatttgaataaaaacgacttcctcatactgtaaacatcacgcatgcacattagacaataatccagtaacgaaacggcttcagtcgtaatcgcccatatagttggctacagaatgttggatagagaacttaagtacgctaaggcggcgtattttcttaatatttaggggtattgccgcagtcattacagataaccgcctatgcggccatgccaggattatagataactttttaacattagccgcagaggtgggactagcacgtaatatcagcacataacgtgtcagtcaggtcatcgacctcgtcggactccgggtgcgaggtcgtgaagctggaatacgagtgaggccgccgaggacgtcaggggggtgtaaagcttccatagagcccaccgcatccccagcatgcctgctattgtcttcccaatcctcccccttgctgtcctgccccaccccaccccccagaatagaatgacacctactcagacaatgcgatgcaatttcctcattttattaggaaaggacagtgggagtggcaccttccagggtcaaggaaggcacgggggaggggcaaacaacagatggctggcaactagaaggcacagtcgaggctgatcagcgggtttaaacgggccctctagactcgacgcggccgctttacttgtacagctcgtccatgccgagagtgatcccggcggcggtcacgaactccagcaggaccatgtgatcgcgcttctcgttggggtctttgctcagggcggactgggtgctcaggtagtggttgtcgggcagcagcacggggccgtcgccgatgggggtgttctgctggtagtggtcggcgagctgcacgctgccgtcctcgatgttgtggcggatcttgaagttcaccttgatgccgttcttctgcttgtcggccatgatatagacgttgtggctgttgtagttgtactccagcttgtgccccaggatgttgccgtcctccttgaagtcgatgcccttcagctcgatgcggttcaccagggtgtcgccctcgaacttcacctcggcgcgggtcttgtagttgccgtcgtccttgaagaagatggtgcgctcctggacgtagccttcgggcatggcggacttgaagaagtcgtgctgcttcatgtggtcggggtagcggctgaagcactgcacgccgtaggtcagggtggtcacgagggtgggccagggcacgggcagcttgccggtggtgcagatgaacttcagggtcagcttgccgtaggtggcatcgccctcgccctcgccggacacgctgaacttgtggccgtttacgtcgccgtccagctcgaccaggatgggcaccaccccggtgaacagctcctcgcccttgctcaccatggtggcgaccggtggggagagaggtcggtgattcggtcaacgagggagccgactgccgacgtgcgctccggaggcttgcagaatgcggaacaccgcgcgggcaggaacagggcccacactaccgccccacaccccgcctcccgcaccgccccttcccggccgctgctctcggcgcgccctgctgagcagccgctattggccacagcccatcgcggtcggcgcgctgccattgctccctggcgctgtccgtctgcgagggtactagtgagacgtgcggcttccgtttgtcacgtccggcacgccgcgaaccgcaaggaaccttcccgacttaggggcggagcaggaagcgtcgccggggggcccacaagggtagcggcgaagatccgggtgacgctgcgaacggacgtgaagaatgtgcgagacccagggtcggcgccgctgcgtttcccggaaccacgcccagagcagccgcgtccctgcgcaaacccagggctgccttggaaaaggcgcaaccccaaccccgtggaagcttgcgacctggaatcggacagcagcggggagtgtacggccccgagttcgtgaccgggtatgctttcattgtaccccggaactttaaatctatgaacaatcgcaacaaattgtccaaaggcaacaatacgacacagttagaggccatcggcgcaggtacactctatccacgcctatcagaatgtcacctggttaatggtcaatttaggtggctggaggcacatgtgaagcaatatggtctagggaaagatatcggtttacttagattttatagttccggatccaacttaaataatataggtattaaagagcagtatcaagagggtttcttcccaaggaatcttgcgattttcatacacagctttaacaaatttcactagacgcaccttcattttgtcgtctcgttgtatatgagtccggggtaagaattttttaccgtatttaacatgatcaacgggtactaaagcaatgtcatttctaaacacagtaggtaaaggacacgtcatcttattttaaagaatgtcagaaatcagggagactagatcgatattacgtgtttt

SEQ ID NO: 106 - DNA, 23 bp, synthetic construct
  misc_feature 1..23: note = Description of Artificial Sequence: Synthetic primer
  source 1..23: mol_type = other DNA
gcatttcaggtttccttgagtgg

SEQ ID NO: 107 - DNA, 21 bp, synthetic construct
  misc_feature 1..21: note = Description of Artificial Sequence: Synthetic primer
  source 1..21: mol_type = other DNA
gcactgttgctcttgaagtcc

SEQ ID NO: 108 - DNA, 281 bp, synthetic construct
  misc_feature 1..281: note = Description of Artificial Sequence: Synthetic polynucleotide
  source 1..281: mol_type = other DNA
caggccaggcctggcgtgaacgttcactgaaatcatggcctcttggccaagattgatagcttgtgcctgtccctgagtcccagtccatcacgagcagctggtttctaagatgctatttcccgtataaagcatgagaccgtgacttgccagccccacagagccccgcccttgtccatcactggcatctggactccagcctgggttggggcaaagagggaaatgagatcatgtcctaaccctgatcctcttgtcccacagatatccagaaccctgaccctgcc

SEQ ID NO: 109 - DNA, 140 bp, synthetic construct
  misc_feature 1..140: note = Description of Artificial Sequence: Synthetic polynucleotide
  source 1..140: mol_type = other DNA
gtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctat

SEQ ID NO: 110 - DNA, 22 bp, synthetic construct
  misc_feature 1..22: note = Description of Artificial Sequence: Synthetic primer
  source 1..22: mol_type = other DNA
cttgtccatcactggcatctgg

SEQ ID NO: 111 - DNA, 21 bp, synthetic construct
  misc_feature 1..21: note = Description of Artificial Sequence: Synthetic primer
  source 1..21: mol_type = other DNA
tttggtggcaatggataaggc

SEQ ID NO: 112 - DNA, 142 bp, synthetic construct
  misc_feature 1..142: note = Description of Artificial Sequence: Synthetic polynucleotide
  source 1..142: mol_type = other DNA
actccagcctgggttggggcaaagagggaaatgagatcatgtcctaaccctgatcctcttgtcccacagatatccagaaccctgaccctgccgtgtaccagctgagagactctaaatccagtgacaagtctgtctgcctatt

SEQ ID NO: 113 - DNA, 311 bp, synthetic construct
  misc_feature 1..311: note = Description of Artificial Sequence: Synthetic polynucleotide
  source 1..311: mol_type = other DNA
caccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccaggtaagggcagctttggtgccttcgcaggctgtttccttgcttcaggaatggccaggttctgcccagagctctggtcaatgatgtctaaaactcctctgattggtggtctcg

SEQ ID NO: 114 - DNA, 23 bp, synthetic construct
  misc_feature 1..23: note = Description of Artificial Sequence: Synthetic primer
  source 1..23: mol_type = other DNA
tcctaaccctgatcctcttgtcc

SEQ ID NO: 115 - DNA, 21 bp, synthetic construct
  misc_feature 1..21: note = Description of Artificial Sequence: Synthetic primer
  source 1..21: mol_type = other DNA
ccgtgtcattctctggactgc

SEQ ID NO: 116 - DNA, 188 bp, synthetic construct
  misc_feature 1..188: note = Description of Artificial Sequence: Synthetic polynucleotide
  source 1..188: mol_type = other DNA
cacagatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtg

SEQ ID NO: 117 - DNA, 259 bp, synthetic construct
  misc_feature 1..259: note = Description of Artificial Sequence: Synthetic polynucleotide
  source 1..259: mol_type = other DNA
ctgtggcctggagcaacaaatctgactttgcatgtgcaaacgccttcaacaacagcattattccagaagacaccttcttccccagcccaggtaagggcagctttggtgccttcgcaggctgtttccttgcttcaggaatggccaggttctgcccagagctctggtcaatgatgtctaaaactcctctgattggtggtctcggccttatccattgccaccaaaaccctctttttactaagaaacagtgagccttgttctg

SEQ ID NO: 118 - RNA, 20 nt, synthetic construct
  misc_feature 1..20: note = Description of Artificial Sequence: Synthetic oligonucleotide
  source 1..20: mol_type = other RNA
cagacttctccacaggagtc